(19) United States
(12) Patent Application Publication
(10) Pub. No.: US 2025/0051778 A1
(43) Pub. Date: Feb. 13, 2025

(54) OLIGONUCLEOTIDE COMPOSITIONS AND METHODS OF USE THEREOF

(71) Applicant: WAVE LIFE SCIENCES LTD., Singapore (SG)

(72) Inventors: Michael John Byrne, Natick, MA (US); Vinod Vathipadiekal, Stoneham, MA (US); Naoki Iwamoto, Boston, MA (US); Chandra Vargeese, Schwenksville, PA (US); Lankai Guo, Winchester, MA (US)

(21) Appl. No.: 17/605,998

(22) PCT Filed: Apr. 24, 2020
(86) PCT No.: PCT/US20/29957

§ 371 Date: Oct. 22, 2021

Related U.S. Application Data
(60) Provisional application No. 62/905,323, filed on Sep. 24, 2019.
(60) Provisional application No. 62/838,701, filed on Apr. 25, 2019.

(51) Int. Cl. C12N 15/113

(52) U.S. Cl. CPC C12N 15/1136; C12N 2310/11; C12N 2310/315; C12N 2320/33

(57) ABSTRACT

Among other things, the present disclosure provides USH2A oligonucleotides, and compositions and methods of use thereof, for preventing and/or treating various conditions, disorders or diseases. In some embodiments, provided USH2A oligonucleotides comprise nucleobase modifications, sugar modifications, internucleotidic linkage modifications and/or patterns thereof, and have improved properties, activities and/or selectivities. In some embodiments, the present disclosure provides USH2A oligonucleotides, compositions and methods for preventing and/or treating USH2A-related conditions, disorders or diseases, such as Usher Syndrome (e.g., Usher Syndrome Type 2A), atypical Usher syndrome, or nonsyndromic retinitis pigmentosa.

### CROSS-REFERENCE TO RELATED APPLICATIONS

This application is a National Stage Entry of PCT Application No. PCT/US2020/029957, filed Apr. 24, 2020 and published Oct. 29, 2020 as WO 2020/219981, which claims priority to United States Provisional Application Nos. 62/838,701, filed Apr. 25, 2019, and 62/905,323, filed Sep. 24, 2019, the entirety of each of which is incorporated herein by reference.

### SEQUENCE LISTING

The instant application contains a Sequence Listing which has been submitted electronically in ASCII format and is hereby incorporated by reference in its entirety. Said ASCII copy, created on Jun. 1, 2020, is named Sequence_Listing.txt and is 65,897 bytes in size.

### BACKGROUND

Oligonucleotides are useful in various applications, e.g., therapeutic, diagnostic, and/or research applications. For example, oligonucleotides targeting various genes can be useful for treatment of conditions, disorders or diseases related to such target genes.

### SUMMARY

Among other things, the present disclosure provides USH2A oligonucleotides and compositions, and technologies for designing, manufacturing and utilizing USH2A oligonucleotides and compositions, including but not limited to those capable of mediating skipping of a deleterious exon in an USH2A transcript. Particularly, in some embodiments, the present disclosure provides oligonucleotides and compositions of oligonucleotides that comprise useful patterns of internucleotidic linkages [e.g., types, modifications, and/or configuration (Rp or Sp) of chiral linkage phosphorus, etc.] which, when combined with one or more other structural elements described herein, e.g., nucleobase modifications (and patterns thereof), sugar modifications (and patterns thereof), additional chemical moieties (and patterns thereof), etc., can provide oligonucleotides and compositions with high activities and/or various desired properties, e.g., high efficiency of skipping of a deleterious exon, high selectivity, low toxicity, etc.

In some embodiments, the present disclosure provides technologies (e.g., oligonucleotides, compositions, methods, etc.) for increasing levels of beneficial USH2A gene products (e.g., transcripts, proteins, etc.), e.g., mediated by skipping of a deleterious exon in a mutant USH2A transcript. Among other things, provided technologies can provide various advantages, such as high efficiency of skipping of a deleterious exon, high selectivity (e.g., less skipping of other exons, and/or less off-target effects), and/or high activities (e.g., skipping of a deleterious exon in an USH2A gene transcript at low concentrations and/or high level of desired skipping at certain concentrations).

In some embodiments, a target nucleic acid of a provided oligonucleotide is an USH2A transcript (e.g., a mutant USH2A mRNA) that comprises a disease-associated mutation (e.g., a disease-associated mutation in a particular exon, including but not limited to exon 13) and is associated with a condition, disorder or disease [e.g., Usher Syndrome (e.g., Usher Syndrome Type 2A), atypical Usher syndrome, or nonsyndromic retinitis pigmentosa].

Pathogenic mutations in the USH2A gene reportedly disrupt the production of the USH2A protein (also known as usherin), one of the proteins expressed in the photoreceptors where it is required for their maintenance. Pathogenic mutations in the USH2A gene reportedly cause retinitis pigmentosa (RP) (e.g., autosomal recessive retinitis pigmentosa, or ARRP or arRP) and Usher Syndrome Type IIA (2A), and atypical Usher Syndrome.

Mutations in USH2A exon 13 are reportedly present in both non-syndromic and syndromic forms of RP. Exon 13 mutations are reportedly some of the most common USH2A mutations. Mutations in exon 13 of the USH2A gene reportedly result in the absence of the usherin protein and loss of usherin protein activity in the retinal photoreceptors and degeneration of the outer segment of photoreceptor cells.

Without wishing to be bound by any particular theory, the present disclosure encompasses the recognition that treatment of a patient in need thereof with an USH2A oligonucleotide capable of skipping (e.g., exclusion of) a deleterious exon (including but not limited to exon 13) in an USH2A gene transcript can result in production of an internally truncated but at least partially functional USH2A protein, which can in turn result in restoration of at least partial usherin protein activity in photoreceptors and at least partial restoration of vision in patients with RP due to mutations in the deleterious exon of the USH2A gene. In some embodiments, an internally truncated USH2A protein, e.g., as a product of skipping of exon 13 which comprises one or more deleterious mutations, provides certain functions and activities of a wild-type USH2A protein, either fully or partially. In some embodiments, the present disclosure provides oligonucleotides and compositions thereof, including chirally controlled oligonucleotide compositions thereof, that when administered into a cell and/or a subject can provide exon 13 skipped USH2A transcripts (e.g., mRNA) and proteins encoded thereby. In some embodiments, as described herein, the present disclosure provides methods for using such oligonucleotides and compositions, e.g., for preventing, slowing the onset, development and/or progress, and/or treating a condition, disorder or disease associated with exon 13 of USH2A (e.g., associated with one or more mutations in exon 13 which can cause loss of, or loss of one or more or all functions of, normal USH2A proteins).

In some embodiments, an USH2A oligonucleotide, e.g., one capable of mediating skipping of USH2A exon 13, has a sequence which hybridizes to (e.g., is complementary to a sequence of) an USH2A gene transcript sequence within exon 13, a sequence within an intron immediately adjacent to exon 13 (e.g., intron 12 or intron 13), or a sequence spanning the boundary between USH2A exon 13 and an intron immediately adjacent to exon 13 (e.g., spanning the boundary between exon 13 and intron 12, or the spanning the boundary between exon 13 and intron 13). In some embodiments, the boundaries between exon 13 and the introns immediately 5′ or 3′ to exon 13 are reported in Weston et al. Am. J. Hum. Genet. 66:1199-1210, 2000.

In some embodiments, the base sequence of an USH2A oligonucleotide is, comprises, comprises at least 15 contiguous bases of, comprises at least 15 contiguous bases of (with 0 to 3 mismatches), or comprises at least 10 contiguous bases (e.g., 10-15, 10-20, 10, 11, 12, 13, 14, 15, 16, 17, 18, 19, or 20), of the base sequence of: AAGCCCUAAAGAUAAAAUAU (SEQ ID NO: 1), AAUACAUUUCUUUCUUACCU (SEQ ID NO: 2), ACAUCCAACAUCAUUAAAGC (SEQ ID NO: 3), AGCUUCGGAGAAAUUUAAAUC (SEQ ID NO: 4), AGCUUCGGAGAAAUUUAAAUC (SEQ ID NO: 5), AGGAUUGCAGAAUUUGUUCA (SEQ ID NO: 6), AGGAUUGCAGAAUUUGUUCA (SEQ ID NO: 7), AUCCAAAAUUGCAAUGAUCA (SEQ ID NO: 8), AUUUCUUUCUUACCUGGUUG (SEQ ID NO: 9), CAACAUCAUUAAAGCUUCGG (SEQ ID NO: 10), CACCUAAGCCCUAAAGAUAA (SEQ ID NO: 11), GAGGAUUGCAGAAUUUGUUC (SEQ ID NO: 12), GAUCACACCUAAGCCCUAAA (SEQ ID NO: 13), GAUUGCAGAAUUUGUUCACU (SEQ ID NO: 14), GCAAUGAUCACACCUAAGCC (SEQ ID NO: 15), GCUUCGGAGAAAUUUAAAUC (SEQ ID NO: 16), GGAAUCACACUCACACAUCU (SEQ ID NO: 17), GGAUUGCAGAAUUUGUUCAC (SEQ ID NO: 18), GGAUUGCAGAAUUUGUUCA (SEQ ID NO: 19), UACCUGGUUGACACUGAUUA (SEQ ID NO: 20), UACCUGGUUGACACUGAUUA (SEQ ID NO: 21), UCUUUUUUGCACUCACACUG (SEQ ID NO: 22), UGAGGAUUGCAGAAUUUGUU (SEQ ID NO: 23), UGAGGAUUGCAGAAUUUGUU (SEQ ID NO: 24), UGCAGAAUUUGUUCACUGAG (SEQ ID NO: 25), UUGCAGAAUUUGUUCACUGA (SEQ ID NO: 26), or UUUCUUACCUGGUUGACACU (SEQ ID NO: 27), wherein each U may be optionally and independently replaced with T. In some embodiments, such an oligonucleotide is an USH2A oligonucleotide which targets a mutant USH2A gene transcript (e.g., an oligonucleotide whose base sequence is complementary to a base sequence in the mutant USH2A target gene transcript). In some embodiments, such an oligonucleotide is capable of mediating skipping of USH2A exon 13. In some embodiments, a base sequence of a provided oligonucleotide is or comprises GGAUUGCAGAAUUUGUUCAC (SEQ ID NO: 18). In some embodiments, the base sequence of a provided oligonucleotide is or comprises GAUUGCAGAAUUUGUUCACU (SEQ ID NO: 14). As demonstrated herein, oligonucleotides whose base sequences are or comprise such sequences can be particularly useful.

In some embodiments, provided oligonucleotides can provide high levels of exon skipping, and/or high selectivity for skipping of particular exons (e.g., in some embodiments, high selectivity for skipping exon 13 only (low levels of skipping other exon(s), e.g., exon 12, exon 12 and exon 13, etc.)).

In some embodiments, the sequence of a provided USH2A oligonucleotide is fully complementary to a target nucleic acid sequence at a particular site, e.g., the sequence of the USH2A oligonucleotide is fully complementary to one or more mutant sites of an USH2A transcript. In some embodiments, a mutant site is in exon 13 of USH2A.

In some embodiments, provided oligonucleotides and compositions are useful for preventing and/or treating various conditions, disorders or diseases, particularly USH2A-related conditions, disorders or diseases, including Usher Syndrome (e.g., Usher Syndrome Type 2A), atypical Usher syndrome, and nonsyndromic retinitis pigmentosa. In some embodiments, provided oligonucleotides and compositions reduce levels of an USH2A transcript (e.g., mRNA) and/or a product encoded thereby, for example, a transcript comprising a deleterious exon (e.g., exon 13), and/or a protein comprising a deleterious mutation. In some embodiments, provided oligonucleotides and compositions increase levels of USH2A transcripts and/or products encoded thereby, which USH2A transcripts have an exon skipped (e.g., exon 13) and encode products (e.g., protein) that can provide one or more desirable functions at higher levels compared to those encoded by transcripts without the exon skipped. In some embodiments, as described herein, a skipped exon, e.g., exon 13, comprises one or more mutations associated with conditions, disorders or diseases, such as Usher Syndrome (e.g., Usher Syndrome Type 2A), atypical Usher syndrome, and nonsyndromic retinitis pigmentosa. In some embodiments, provided oligonucleotides and compositions selectively increase levels of USH2A transcripts and/or products encoded thereby that are capable of treating, ameliorating or delaying at least one symptom associated with Usher Syndrome (e.g., Usher Syndrome Type 2A), atypical Usher syndrome, or nonsyndromic retinitis pigmentosa, wherein a deleterious exon (e.g., exon 13) in the USH2A transcript has been skipped, and the product thereof is an internally truncated USH2A protein capable of performing at least one function of USH2A.

In some embodiments, methods and compositions described herein, provide for treating or delaying the onset or progression of a disease, disorder or condition of the eye, e.g., a disorder that affects retinal cells, e.g., photoreceptor cells, or of the ear, that is related to USH2A. In some embodiments, methods and compositions discussed herein, provide for treating or delaying the onset or progression of a disease, disorder or condition associated with an USH2A mutation, e.g., by administering a therapeutic amount of a USH2A oligonucleotide. In some embodiments, provided oligonucleotides are oligonucleotides targeting USH2A, and can skip a deleterious exon (e.g., exon 13) of an USH2A gene transcript. In some embodiments, a USH2A oligonucleotide is useful for preventing, treating or delaying the onset or progression of an USH2A-related condition, disorder and/or disease, including retinopathy (e.g, retinal degeneration, retinal degenerative disease, retinal degenerative disorder, inherited retinal degenerative disorder, retinitis pigmentosa, autosomal dominant retinitis pigmentosa, etc.).

Among other things, the present disclosure encompasses the recognition that controlling structural elements of USH2A oligonucleotides can have a significant impact on oligonucleotide properties and/or activities, including but not limited to increasing the level of skipping of a deleterious exon in an USH2A target gene transcript. In some embodiments, controlled structural elements of USH2A oligonucleotides include but are not limited to: base sequence, chemical modifications (e.g., modifications of a sugar, base and/or internucleotidic linkage) or patterns thereof, alterations in stereochemistry (e.g., stereochemistry of a backbone chiral internucleotidic linkage) or patterns thereof, and/or conjugation with an additional chemical moiety (e.g., a carbohydrate moiety, a targeting moiety, etc.). Particularly, in some embodiments, the present disclosure demonstrates that control of stereochemistry of backbone chiral centers (stereochemistry of linkage phosphorus), optionally with controlling other aspects of oligonucleotide design and/or incorporation of carbohydrate moieties, can greatly improve properties and/or activities of USH2A oligonucleotides, including but not limited to, their ability to mediate skipping of a deleterious exon in an USH2A transcript.

In some embodiments, the present disclosure pertains to any USH2A oligonucleotide which operates through any mechanism, and which comprises any sequence, structure or format (or portion thereof) described herein, wherein the oligonucleotide comprises at least one non-naturally-occurring modification of a base, sugar or internucleotidic linkage.

In some embodiments, the present disclosure provides an oligonucleotide composition comprising a plurality of oligonucleotides, wherein the oligonucleotides comprise at least one (e.g., 1-100, 1-50, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45 or more) chirally controlled internucleotidic linkage [an internucleotidic linkage whose linkage phosphorus is in or is enriched for the Rp or Sp configuration (e.g., 80-100%, 85%-100%, 90%-100%, 95%-100%, or 85%, 90%, 91%, 92%, 93%, 94%, 95%, 96%, 97%, 98%, 99% or more of all oligonucleotides of the same constitution in the composition share the same stereochemistry at the linkage phosphorus) but not a random mixture of the Rp and Sp, such an internucleotidic linkage also a “stereodefined internucleotidic linkage”, and such an oligonucleotide composition also a “stereodefined oligonucleotide composition”], e.g., a phosphorothioate linkage whose linkage phosphorus is Rp or Sp. In some embodiments, the number of chirally controlled internucleotidic linkages is 1-100, 1-50, 1-40, 1-35, 1-30, 1-25, 1-20, 5-100, 5-50, 5-40, 5-35, 5-30, 5-25, 5-20, 1, 2, 3, 4, 5, 6, 7, 8, 9, 10, 11, 12, 13, 14, 15, 16, 17, 18, 19, 20, 21, 22, 23, 24, or 25. In some embodiments, at least 5%10%, 15%, 20%, 25%, 30%, 35%, 40%, 45%, 50%, 55%, 60%, 65%, 70%, 75%, 80%, 85%, 90%, or 95%, or 100% of all chiral internucleotidic linkages are chirally controlled internucleotidic linkages. In some embodiments, at least 5%, 10%, 15%, 20%, 25%, 30%, 35%, 40%, 45%, 50%, 55%, 60%, 65%, 70%, 75%, 80%, 85%, 90%, or 95%, or 100% of all internucleotidic linkages are chirally controlled internucleotidic linkages. In some embodiments, at least 5%, 10%, 15%, 20%, 25%, 30%, 35%, 40%, 45%, 50%, 55%, 60%, 65%, 70%, 75%, 80%, 85%, 90%, or 95%, or 100% of all chiral internucleotidic linkages are chirally controlled internucleotidic linkages and are Sp. In some embodiments, at least 5%, 10%, 15%, 20%, 25%, 30%, 35%, 40%, 45%, 50%, 55%, 60%, 65%, 70%, 75%, 80%, 85%, 90%, or 95%, or 100% of all internucleotidic linkages are chirally controlled internucleotidic linkages and are Sp. In some embodiments, at least 1 internucleotidic linkage is chirally controlled internucleotidic linkage and is Rp. In some embodiments, at least 2 internucleotidic linkages are chirally controlled internucleotidic linkage and are Rp. In some embodiments, at least 3 internucleotidic linkages are chirally controlled internucleotidic linkage and are Rp. In some embodiments, at least 4 internucleotidic linkages are chirally controlled internucleotidic linkage and are Rp. In some embodiments, at least 5 internucleotidic linkages are chirally controlled internucleotidic linkage and are Rp. In some embodiments, each chiral internucleotidic linkage is independently a chirally controlled internucleotidic linkage. In some embodiments, each chirally controlled internucleotidic linkage is Sp.

In some embodiments, the present disclosure pertains to an USH2A oligonucleotide composition wherein the USH2A oligonucleotides comprise at least one chiral internucleotidic linkage which is not chirally controlled (including but not limited to: a phosphorothioate which is not chirally controlled). In some embodiments, the present disclosure pertains to an USH2A oligonucleotide composition wherein the USH2A oligonucleotides are stereorandom.

In some embodiments, oligonucleotides comprise one or more (e.g., 1, 2, 3, 4, 5, 6, 7, 8, 9, or 10) non-negatively charged internucleotidic linkages. In some embodiments, oligonucleotides comprise one or more (1, 2, 3, 4, 5, 6, 7, 8, 9, or 10) neutral internucleotidic linkages. In some embodiments, an USH2A oligonucleotide comprises a non-negatively charged or neutral internucleotidic linkage. In some embodiments, the present disclosure provides an oligonucleotide, wherein the base sequence of the oligonucleotide comprises at least 10 contiguous bases of a base sequence that is identical to or complementary to a base sequence of an USH2A gene or a transcript thereof, wherein the oligonucleotide comprises at least one internucleotidic linkage comprising a stereodefined linkage phosphorus, and wherein the oligonucleotide is capable of increasing the level of skipping of a deleterious exon in a mutant USH2A gene transcript or a gene product thereof (e.g., increasing the level of an USH2A protein translated from an USH2A gene transcript in which a deleterious exon has been skipped, wherein the protein is internally truncated and performs at least one function of USH2A).

In some embodiments, various optional additional chemical moieties, such as carbohydrate moieties, targeting moieties, etc., can be incorporated into oligonucleotides, and can improve one or more properties and/or activities.

In some embodiments, an additional chemical moiety is selected from: GalNAc, glucose, GluNAc (N-acetyl amine glucosamine) and anisamide moieties and derivatives thereof, or any additional chemical moiety described herein and/or known in the art. In some embodiments, an oligonucleotide can comprise two or more additional chemical moieties, wherein the additional chemical moieties are identical or non-identical, or are of the same category (e.g., carbohydrate moiety, sugar moiety, targeting moiety, etc.) or not of the same category. In some embodiments, certain additional chemical moieties facilitate delivery of oligonucleotides to desired cells, tissues and/or organs. In some embodiments, certain additional chemical moieties facilitate internalization of oligonucleotides. In some embodiments, certain additional chemical moieties increase oligonucleotide stability.

In some embodiments, the present disclosure provides a chirally controlled USH2A oligonucleotide composition comprising a plurality of oligonucleotides which share:
1) a common base sequence;
2) a common pattern of backbone linkages; and
3) a common pattern of backbone chiral centers, which composition is a substantially pure preparation of a single oligonucleotide in that a non-random or controlled level of the oligonucleotides in the composition have the common base sequence, the common pattern of backbone linkages, and the common pattern of backbone chiral centers. In some embodiments, the chirally controlled oligonucleotide composition is capable of mediating skipping of a deleterious exon in a mutant USH2A gene transcript.

In some embodiments, an USH2A oligonucleotide composition is a chirally controlled oligonucleotide composition comprising a plurality of oligonucleotides of a particular oligonucleotide type, which composition is enriched, relative to a substantially racemic preparation of oligonucleotides having the same base sequence, for oligonucleotides of the particular oligonucleotide type. In some embodiments, such a composition is capable of mediating skipping of a deleterious exon in a mutant USH2A gene transcript.

In some embodiments, the present disclosure provides a chirally controlled oligonucleotide composition comprising a plurality of oligonucleotides, wherein oligonucleotides of the plurality share the same constitution and comprise at least one (e.g., 1-100, 1-50, 1, 2, 3, 4, 5, 6, 7, 8, 9, 10, 11, 12, 13, 14, 15, 16, 17, 18, 19, 20, 21, 22, 23, 24, or 25 or more) chirally controlled internucleotidic linkage. In some embodiments, oligonucleotides of the plurality are USH2A oligonucleotides whose base sequences is identical to or complementary to a sequence of an USH2A gene or a product thereof (e.g., a RNA transcript). In some embodiments, oligonucleotides of the plurality are capable of hybridizing to an USH2A gene transcript and mediating skipping of a deleterious exon in a mutant USH2A gene transcript.

In some embodiments, the present disclosure provides a chirally controlled oligonucleotide composition comprising a plurality of oligonucleotides capable of mediating skipping of a deleterious exon in an USH2A transcript, wherein oligonucleotides of the plurality are of a particular oligonucleotide type, which composition is enriched, relative to a substantially racemic preparation of oligonucleotides having the same base sequence, for oligonucleotides of the particular oligonucleotide type. In some embodiments, oligonucleotides of the same oligonucleotide type have the same structure.

In some embodiments, an oligonucleotide or oligonucleotide composition is useful for preventing or treating a condition, disorder or disease. In some embodiments, an USH2A oligonucleotide or USH2A oligonucleotide composition is useful for a method of treatment of an USH2A-related condition, disorder or disease, such as Usher Syndrome (e.g., Usher Syndrome Type 2A), atypical Usher syndrome, or nonsyndromic retinitis pigmentosa, in a subject in need thereof.

In some embodiments, an oligonucleotide or oligonucleotide composition is useful for the manufacture of a medicament for treatment of a condition, disorder or disease, such as Usher Syndrome (e.g., Usher Syndrome Type 2A), atypical Usher syndrome, or nonsyndromic retinitis pigmentosa, in a subject in need thereof. In some embodiments, an USH2A oligonucleotide or USH2A oligonucleotide composition is useful for the manufacture of a medicament for treatment of an USH2A-related condition, disorder or disease, such as Usher Syndrome (e.g., Usher Syndrome Type 2A), atypical Usher syndrome, or nonsyndromic retinitis pigmentosa, in a subject in need thereof.

In some embodiments, the present disclosure provides a pharmaceutical composition comprising a therapeutically effective amount of a provided oligonucleotide, which is optionally in a salt form. In some embodiments, an oligonucleotide is provided as its sodium salt form. In some embodiments, a pharmaceutical composition further comprises a pharmaceutically acceptable carrier.

In some embodiments, the present disclosure provides methods for preventing, delaying the onset and/or development of, and/or treating a condition, disorder or disease, comprising administering to a subject susceptible thereto or suffering therefrom an effective amount of a provided oligonucleotide or a composition thereof. In some embodiments, a condition, disorder or disease is associated with an USH2A mutation. In some embodiments, a condition, disorder or disease is associated with an USH2A mutation in exon 13. In some embodiments, a condition, disorder or disease is Usher Syndrome (e.g., Usher Syndrome Type 2A), atypical Usher syndrome, or nonsyndromic retinitis pigmentosa. In some embodiments, an administered oligonucleotide can provide skipping of exon 13 in an USH2A transcript, and a transcript without exon 13 (e.g., mRNA) can provide a product, e.g., a protein, that can provide higher levels of one or more desired biological functions compared to the corresponding transcript with exon 13.

### BRIEF DESCRIPTION OF THE DRAWINGS

FIG. 1. Certain useful mouse models for assessing provided technology.

FIG. 2A and FIG. 2B. Provided technologies can provide efficient exon skipping in vivo. Data are posterior of the eye (retina, choroid, sclera) 1 week post single IVT injection. As demonstrated in FIG. 2A, provided technologies, e.g. chirally controlled oligonucleotide compositions of WV-20902, WV-24360 and WV-30205, are significantly more effective than reference conditions, e.g., absence of oligonucleotides (PBS) and presence of a reference stereorandom composition (WV-20781). FIG. 2B illustrated tissue exposure (note in this set of data in FIG. 2B, results for WV-20781 may not reflect actual exposure level due to assay conditions).

FIG. 3A and FIG. 3B. Provided technologies can provide efficient exon skipping in vivo. As demonstrated, chirally controlled oligonucleotide compositions (WV-20902, WV-24360 and WV-30205) provided significantly higher levels of exon skipping compared to a reference stereorandom oligonucleotide composition (WV-20781). Certain data at 1 week (FIG. 3A and FIG. 3B) and through 8 weeks (FIG. 3B) were shown as examples. Presented data were exon skipping data in retina, single IVT injection in non-human primate models.

FIG. 4A and FIG. 4B. Provided technologies can provide efficient exon skipping in vivo. FIG. 4A demonstrates that provided technologies, e.g., as illustrated by chirally controlled oligonucleotide compositions of WV-30205, can provide dose-dependent, dramatically higher levels of exon skipping at lower or comparable dose levels compared to references, a reference stereorandom composition (WV-20781). FIG. 4B demonstrates that provided technologies can effectively deliver oligonucleotides to target locations. Data were collected from non-human primate model, retina, 1-week following single IVT injection.

### DETAILED DESCRIPTION OF CERTAIN EMBODIMENTS

Technologies of the present disclosure may be understood more readily by reference to the following detailed description of certain embodiments.

### Definitions

As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001.

As used herein in the present disclosure, unless otherwise clear from context, (i) the term “a” or “an” may be understood to mean “at least one”; (ii) the term “or” may be understood to mean “and/or”; (iii) the terms “comprising”, “comprise”, “including” (whether used with “not limited to” or not), and “include” (whether used with “not limited to” or not) may be understood to encompass itemized components or steps whether presented by themselves or together with one or more additional components or steps; (iv) the term “another” may be understood to mean at least an additional/second one or more; (v) the terms “about” and “approximately” may be understood to permit standard variation as would be understood by those of ordinary skill in the art; and (vi) where ranges are provided, endpoints are included.

Unless otherwise specified, description of oligonucleotides and elements thereof (e.g., base sequence, sugar modifications, internucleotidic linkages, linkage phosphorus stereochemistry, etc.) is from 5′ to 3′. Unless otherwise specified, oligonucleotides described herein may be provided and/or utilized in salt forms, particularly pharmaceutically acceptable salt forms, e.g., sodium salts. As those skilled in the art will appreciate, in some embodiments, individual oligonucleotides within a composition may be considered to be of the same constitution and/or structure even though, within such composition (e.g., a liquid composition), particular such oligonucleotides might be in different salt form(s) (and may be dissolved and the oligonucleotide chain may exist as an anion form when, e.g., in a liquid composition) at a particular moment in time. For example, those skilled in the art will appreciate that, at a given pH, individual internucleotidic linkages along an oligonucleotide chain may be in an acid (H) form, or in one of a plurality of possible salt forms (e.g., a sodium salt, or a salt of a different cation, depending on which ions might be present in the preparation or composition), and will understand that, so long as their acid forms (e.g., replacing all cations, if any, with H+) are of the same constitution and/or structure, such individual oligonucleotides may properly be considered to be of the same constitution and/or structure (and share the same pattern of backbone linkages and/or pattern of backbone chiral centers).

Aliphatic: As used herein, “aliphatic” means a straight-chain (i.e., unbranched) or branched, substituted or unsubstituted hydrocarbon chain that is completely saturated or that contains one or more units of unsaturation, or a substituted or unsubstituted monocyclic, bicyclic, or polycyclic hydrocarbon ring that is completely saturated or that contains one or more units of unsaturation (but not aromatic), or combinations thereof. In some embodiments, aliphatic groups contain 1-50 aliphatic carbon atoms. In some embodiments, aliphatic groups contain 1-20 aliphatic carbon atoms. In other embodiments, aliphatic groups contain 1-10 aliphatic carbon atoms. In other embodiments, aliphatic groups contain 1-9 aliphatic carbon atoms. In other embodiments, aliphatic groups contain 1-8 aliphatic carbon atoms. In other embodiments, aliphatic groups contain 1-7 aliphatic carbon atoms. In other embodiments, aliphatic groups contain 1-6 aliphatic carbon atoms. In still other embodiments, aliphatic groups contain 1-5 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

Alkenyl: As used herein, the term “alkenyl” refers to an aliphatic group, as defined herein, having one or more double bonds.

Alkyl: As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, alkyl has 1-100 carbon atoms. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In some embodiments, cycloalkyl rings have from about 3-10 carbon atoms in their ring structure where such rings are monocyclic, bicyclic, or polycyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

Alkynyl: As used herein, the term “alkynyl” refers to an aliphatic group, as defined herein, having one or more triple bonds.

Analog: The term “analog” includes any chemical moiety which differs structurally from a reference chemical moiety or class of moieties, but which is capable of performing at least one function of such a reference chemical moiety or class of moieties. As non-limiting examples, a nucleotide analog differs structurally from a nucleotide but performs at least one function of a nucleotide; a nucleobase analog differs structurally from a nucleobase but performs at least one function of a nucleobase; etc.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate and/or a pig). In some embodiments, animals include, but are not limited to, mammals, birds, reptiles, amphibians, fish and/or worms. In some embodiments, an animal may be a transgenic animal, a genetically-engineered animal and/or a clone.

Antisense: The term “antisense”, as used herein, refers to a characteristic of an oligonucleotide or other nucleic acid having a base sequence complementary or substantially complementary to a target nucleic acid to which it is capable of hybridizing. In some embodiments, a target nucleic acid is a target gene mRNA. In some embodiments, hybridization is required for or results in at one activity, e.g., an increase in the level of skipping of a deleterious exon in a target nucleic acid and/or an increase in production of a gene product produced from a target nucleic acid from which a deleterious exon has been skipped. The term “antisense oligonucleotide”, as used herein, refers to an oligonucleotide complementary to a target nucleic acid. In some embodiments, an antisense oligonucleotide is capable of directing an increase in the level of skipping of a deleterious exon in a target nucleic acid and/or increase in production of a gene product produced from a target nucleic acid from which a deleterious exon has been skipped.

Aryl: The term “aryl”, as used herein, used alone or as part of a larger moiety as in “aralkyl,” “aralkoxy,” or “aryloxyalkyl,” refers to monocyclic, bicyclic or polycyclic ring systems having a total of five to thirty ring members, wherein at least one ring in the system is aromatic. In some embodiments, an aryl group is a monocyclic, bicyclic or polycyclic ring system having a total of five to fourteen ring members, wherein at least one ring in the system is aromatic, and wherein each ring in the system contains 3 to 7 ring members. In some embodiments, an aryl group is a biaryl group. The term “aryl” may be used interchangeably with the term “aryl ring.” In certain embodiments of the present disclosure, “aryl” refers to an aromatic ring system which includes, but is not limited to, phenyl, biphenyl, naphthyl, binaphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

Blockmer: the term “blockmer,” as used herein, refers to an oligonucleotide whose pattern of structural features characterizing each individual sugar, nucleobase, internucleotidic linkage, nucleoside, or nucleotide unit is characterized by the presence of at least two consecutive sugar, nucleobase, internucleotidic linkage, nucleoside, or nucleotide units, respectively, sharing a common structural feature. By common structural feature is meant common stereochemistry at the linkage phosphorus, a common modification at the linkage phosphorus, a common modification at the sugar units, a common modification at the nucleobase units, etc. In some embodiments, the at least two units sharing a common structure feature, e.g., at the internucleotidic phosphorus linkage, the sugar, the nucleobase, etc., are referred to as a “block”. In some embodiments, an oligonucleotide is a blockmer.

In some embodiments, a blockmer is a “stereoblockmer,” e.g., at least two consecutive nucleotide units have the same stereochemistry at the linkage phosphorus. Such at least two consecutive nucleotide units form a “stereoblock.”

In some embodiments, a blockmer is a “P-modification blockmer,” e.g., at least two consecutive internucleotidic linkages have the same modification at the linkage phosphorus. Such at least two internucleotidic linkages and the nucleosides connected to them form a “P-modification block”. For instance, (Rp, Sp)-ATsCsGA is a P-modification blockmer because at least two consecutive internucleotidic linkages, the TsC and the CsG, have the same P-modification (i.e., both are a phosphorothioate diester). In the same oligonucleotide of (Rp, Sp)-ATsCsGA, TsCsG forms a block, and it is a P-modification block.

In some embodiments, a blockmer is a “linkage blockmer,” e.g., at least two consecutive internucleotidic linkages have identical stereochemistry and identical modifications at the linkage phosphorus. The at least two consecutive linkages and the nucleosides connected to them form a “linkage block”. For instance, (Rp, Rp)-ATsCsGA is a linkage blockmer because at least two consecutive internucleotidic linkages, the TsC and the CsG, have the same stereochemistry (both Rp) and P-modification (both phosphorothioate). In the same oligonucleotide of (Rp, Rp)-ATsCsGA, TsCsG forms a block, and it is a linkage block.

Chiral control: As used herein, “chiral control” refers to control of the stereochemical designation of the chiral linkage phosphorus in a chiral internucleotidic linkage within an oligonucleotide. As used herein, a chiral internucleotidic linkage is an internucleotidic linkage whose linkage phosphorus is chiral. In some embodiments, a control is achieved through a chiral element that is absent from the sugar and base moieties of an oligonucleotide, for example, in some embodiments, a control is achieved through use of one or more chiral auxiliaries during oligonucleotide preparation as described in the present disclosure, which chiral auxiliaries often are part of chiral phosphoramidites used during oligonucleotide preparation. In contrast to chiral control, a person having ordinary skill in the art appreciates that conventional oligonucleotide synthesis which does not use chiral auxiliaries cannot control stereochemistry at a chiral internucleotidic linkage if such conventional oligonucleotide synthesis is used to form the chiral internucleotidic linkage. In some embodiments, the stereochemical designation of each chiral linkage phosphorus in each chiral internucleotidic linkage within an oligonucleotide is controlled.

Chirally controlled oligonucleotide composition: The terms “chirally controlled oligonucleotide composition”, “chirally controlled nucleic acid composition”, and the like, as used herein, refers to a composition that comprises a plurality of oligonucleotides (or nucleic acids) which share 1) a common base sequence, 2) a common pattern of backbone linkages, and 3) a common pattern of backbone phosphorus modifications, wherein the plurality of oligonucleotides (or nucleic acids) share the same linkage phosphorus stereochemistry at one or more chiral internucleotidic linkages (chirally controlled or stereodefined internucleotidic linkages, whose chiral linkage phosphorus is Rp or Sp in the composition (“stereodefined”), not a random Rp and Sp mixture as non-chirally controlled internucleotidic linkages). Level of the plurality of oligonucleotides (or nucleic acids) in a chirally controlled oligonucleotide composition is pre-determined/controlled (e.g., through chirally controlled oligonucleotide preparation to stereoselectively form one or more chiral internucleotidic linkages). In some embodiments, about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a chirally controlled oligonucleotide composition are oligonucleotides of the plurality. In some embodiments, about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a chirally controlled oligonucleotide composition that share the common base sequence, the common pattern of backbone linkages, and the common pattern of backbone phosphorus modifications are oligonucleotides of the plurality. In some embodiments, a level is about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a composition, or of all oligonucleotides in a composition that share a common base sequence (e.g., of a plurality of oligonucleotide or an oligonucleotide type), or of all oligonucleotides in a composition that share a common base sequence, a common pattern of backbone linkages, and a common pattern of backbone phosphorus modifications, or of all oligonucleotides in a composition that share a common base sequence, a common patter of base modifications, a common pattern of sugar modifications, a common pattern of internucleotidic linkage types, and/or a common pattern of internucleotidic linkage modifications. In some embodiments, the plurality of oligonucleotides share the same stereochemistry at about 1-50 (e.g., about 1-10, 1-20, 5-10, 5-20, 10-15, 10-20, 10-25, 10-30, or about 1, 2, 3, 4, 5, 6, 7, 8, 9, 10, 11, 12, 13, 14, 15, 16, 17, 18, 19, or 20, or at least 1, 2, 3, 4, 5, 6, 7, 8, 9, 10, 11, 12, 13, 14, 15, 16, 17, 18, 19, or 20) chiral internucleotidic linkages. In some embodiments, the plurality of oligonucleotides share the same stereochemistry at about 1%-100% (e.g., about 5%-100%, 10%-100%, 20%-100%, 30%-100%, 40%-100%, 50%-100%, 60%-100%, 70%-100%, 80-100%, 90-100%, 95-100%, 50%-90%, about 5%, 10%, 15%, 20%, 25%, 30%, 35%, 40%, 45%, 50%, 55%, 60%, 65%, 70%, 75%, 80%, 85%, 90%, 95%, or 100%, or at least 5%, 10%, 15%, 20%, 25%, 30%, 35%, 40%, 45%, 50%, 55%, 60%, 65%, 70%, 75%, 80%, 85%, 90%, 95%, or 99%) of chiral internucleotidic linkages. In some embodiments, oligonucleotides (or nucleic acids) of a plurality are of the same constitution. In some embodiments, level of the oligonucleotides (or nucleic acids) of the plurality is about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides (or nucleic acids) in a composition that share the same constitution as the oligonucleotides (or nucleic acids) of the plurality. In some embodiments, each chiral internucleotidic linkage is a chiral controlled internucleotidic linkage, and the composition is a completely chirally controlled oligonucleotide composition. In some embodiments, oligonucleotides (or nucleic acids) of a plurality are structurally identical. In some embodiments, a chirally controlled internucleotidic linkage has a diastereopurity of at least 80%, 85%, 90%, 91%, 92%, 93%, 94%, 95%, 96%, 97%, 98%, 99% or 99.5%, typically at least 90%, 91%, 92%, 93%, 94%, 95%, 96%, 97%, 98%, 99% or 99.5%. In some embodiments, a chirally controlled internucleotidic linkage has a diastereopurity of at least 95%. In some embodiments, a chirally controlled internucleotidic linkage has a diastereopurity of at least 96%. In some embodiments, a chirally controlled internucleotidic linkage has a diastereopurity of at least 97%. In some embodiments, a chirally controlled internucleotidic linkage has a diastereopurity of at least 98%. In some embodiments, a chirally controlled internucleotidic linkage has a diastereopurity of at least 99%. In some embodiments, a percentage of a level is or is at least (DS)^{nc}, wherein DS is a diastereopurity as described in the present disclosure (e.g., 90%, 91%, 92%, 93%, 94%, 95%, 96%, 97%, 98%, 99% or 99.5% or more) and nc is the number of chirally controlled internucleotidic linkages as described in the present disclosure (e.g., 1-50, 1-40, 1-30, 1-25, 1-20, 5-50, 5-40, 5-30, 5-25, 5-20, 1, 2, 3, 4, 5, 6, 7, 8, 9, 10, 11, 12, 13, 14, 15, 16, 17, 18, 19, 20, 21, 22, 23, 24, 25 or more). In some embodiments, a percentage of a level is or is at least (DS)^{nc}, wherein DS is 95%-100%. For example, when DS is 99% and nc is 10, the percentage is or is at least 90% ((99%)¹⁰≈0.90=90%). In some embodiments, level of a plurality of oligonucleotides in a composition is represented as the product of the diastereopurity of each chirally controlled internucleotidic linkage in the oligonucleotides. In some embodiments, diastereopurity of an internucleotidic linkage connecting two nucleosides in an oligonucleotide (or nucleic acid) is represented by the diastereopurity of an internucleotidic linkage of a dimer connecting the same two nucleosides, wherein the dimer is prepared using comparable conditions, in some instances, identical synthetic cycle conditions (e.g., for the linkage between Nx and Ny in an oligonucleotide . . . NxNy . . . , the dimer is NxNy). In some embodiments, not all chiral internucleotidic linkages are chiral controlled internucleotidic linkages, and the composition is a partially chirally controlled oligonucleotide composition. In some embodiments, a non-chirally controlled internucleotidic linkage has a diastereopurity of less than about 80%, 75%, 70%, 65%, 60%, 55%, or of about 50%, as typically observed in stereorandom oligonucleotide compositions (e.g., as appreciated by those skilled in the art, from traditional oligonucleotide synthesis, e.g., the phosphoramidite method). In some embodiments, oligonucleotides (or nucleic acids) of a plurality are of the same type. In some embodiments, a chirally controlled oligonucleotide composition comprises non-random or controlled levels of individual oligonucleotide or nucleic acids types. For instance, in some embodiments a chirally controlled oligonucleotide composition comprises one and no more than one oligonucleotide type. In some embodiments, a chirally controlled oligonucleotide composition comprises more than one oligonucleotide type. In some embodiments, a chirally controlled oligonucleotide composition comprises multiple oligonucleotide types. In some embodiments, a chirally controlled oligonucleotide composition is a composition of oligonucleotides of an oligonucleotide type, which composition comprises a non-random or controlled level of a plurality of oligonucleotides of the oligonucleotide type.

Comparable: The term “comparable” is used herein to describe two (or more) sets of conditions or circumstances that are sufficiently similar to one another to permit comparison of results obtained or phenomena observed. In some embodiments, comparable sets of conditions or circumstances are characterized by a plurality of substantially identical features and one or a small number of varied features. Those of ordinary skill in the art will appreciate that sets of conditions are comparable to one another when characterized by a sufficient number and type of substantially identical features to warrant a reasonable conclusion that differences in results obtained or phenomena observed under the different sets of conditions or circumstances are caused by or indicative of the variation in those features that are varied.

Cycloaliphatic: The term “cycloaliphatic,” “carbocycle,” “carbocyclyl,” “carbocyclic radical,” and “carbocyclic ring,” are used interchangeably, and as used herein, refer to saturated or partially unsaturated, but non-aromatic, cyclic aliphatic monocyclic, bicyclic, or polycyclic ring systems, as described herein, having, unless otherwise specified, from 3 to 30 ring members. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbomyl, adamantyl, and cyclooctadienyl. In some embodiments, a cycloaliphatic group has 3-6 carbons. In some embodiments, a cycloaliphatic group is saturated and is cycloalkyl. The term “cycloaliphatic” may also include aliphatic rings that are fused to one or more aromatic or nonaromatic rings, such as decahydronaphthyl or tetrahydronaphthyl. In some embodiments, a cycloaliphatic group is bicyclic. In some embodiments, a cycloaliphatic group is tricyclic. In some embodiments, a cycloaliphatic group is polycyclic. In some embodiments, “cycloaliphatic” refers to C₃-C₆ monocyclic hydrocarbon, or C₈-C₁₀ bicyclic or polycyclic hydrocarbon, that is completely saturated or that contains one or more units of unsaturation, but which is not aromatic, that has a single point of attachment to the rest of the molecule, or a C₉-C₁₆ polycyclic hydrocarbon that is completely saturated or that contains one or more units of unsaturation, but which is not aromatic, that has a single point of attachment to the rest of the molecule.

Dosing regimen: As used herein, a “dosing regimen” or “therapeutic regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount.

Heteroaliphatic: The term “heteroaliphatic”, as used herein, is given its ordinary meaning in the art and refers to aliphatic groups as described herein in which one or more carbon atoms are independently replaced with one or more heteroatoms (e.g., oxygen, nitrogen, sulfur, silicon, phosphorus, and the like). In some embodiments, one or more units selected from C, CH, CH₂, and CH₃ are independently replaced by one or more heteroatoms (including oxidized and/or substituted forms thereof). In some embodiments, a heteroaliphatic group is heteroalkyl. In some embodiments, a heteroaliphatic group is heteroalkenyl.

Heteroalkyl: The term “heteroalkyl”, as used herein, is given its ordinary meaning in the art and refers to alkyl groups as described herein in which one or more carbon atoms are independently replaced with one or more heteroatoms (e.g., oxygen, nitrogen, sulfur, silicon, phosphorus, and the like). Examples of heteroalkyl groups include, but are not limited to, alkoxy, poly(ethylene glycol)-, alkyl-substituted amino, tetrahydrofuranyl, piperidinyl, morpholinyl, etc.

Heteroaryl: The terms “heteroaryl” and “heteroar-”, as used herein, used alone or as part of a larger moiety, e.g., “heteroaralkyl,” or “heteroaralkoxy,” refer to monocyclic, bicyclic or polycyclic ring systems having a total of five to thirty ring members, wherein at least one ring in the system is aromatic and at least one aromatic ring atom is a heteroatom. In some embodiments, a heteroaryl group is a group having 5 to 10 ring atoms (i.e., monocyclic, bicyclic orpolycyclic), in some embodiments 5, 6, 9, or 10 ring atoms. In some embodiments, a heteroaryl group has 6, 10, or 14 π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic, bicyclic or polycyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl group, wherein the alkyl and heteroaryl portions independently are optionally substituted.

Heteroatom: The term “heteroatom”, as used herein, means an atom that is not carbon or hydrogen. In some embodiments, a heteroatom is boron, oxygen, sulfur, nitrogen, phosphorus, or silicon (including oxidized forms of nitrogen, sulfur, phosphorus, or silicon; charged forms of nitrogen (e.g., quaternized forms, forms as in iminium groups, etc.), phosphorus, sulfur, oxygen; etc.). In some embodiments, a heteroatom is oxygen, sulfur or nitrogen.

Heterocycle: As used herein, the terms “heterocycle,” “heterocyclyl,” “heterocyclic radical,” and “heterocyclic ring”, as used herein, are used interchangeably and refer to a monocyclic, bicyclic or polycyclic ring moiety (e.g., 3-30 membered) that is saturated or partially unsaturated and has one or more heteroatom ring atoms. In some embodiments, a heterocyclyl group is a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes substituted nitrogen. As an example, in a saturated or partially unsaturated ring having 0-3 heteroatoms selected from oxygen, sulfur and nitrogen, the nitrogen may be N (as in 3,4-dihydro-2H-pyrrolyl), NH (as in pyrrolidinyl), or ⁺NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic or polycyclic. The term “heterocyclylalkyl” refers to an alkyl group substituted by a heterocyclyl, wherein the alkyl and heterocyclyl portions independently are optionally substituted.

Identity: As used herein, the term “identity” refers to the overall relatedness between polymeric molecules, e.g., between nucleic acid molecules (e.g., oligonucleotides, DNA, RNA, etc.) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Internucleotidic linkage: As used herein, the phrase “internucleotidic linkage” refers generally to a linkage linking nucleoside units of an oligonucleotide or a nucleic acid. In some embodiments, an internucleotidic linkage is a phosphodiester linkage, as extensively found in naturally occurring DNA and RNA molecules (natural phosphate linkage (—OP(═O)(OH)O—), which as appreciated by those skilled in the art may exist as a salt form). In some embodiments, an internucleotidic linkage is a modified internucleotidic linkage (not a natural phosphate linkage). In some embodiments, an internucleotidic linkage is a “modified internucleotidic linkage” wherein at least one oxygen atom or —OH of a phosphodiester linkage is replaced by a different organic or inorganic moiety. In some embodiments, such an organic or inorganic moiety is selected from ═S, ═Se, ═NR′, —SR′, —SeR′, —N(R′)₂, B(R′)₃, —S—, —Se—, and —N(R′)—, wherein each R′ is independently as defined and described in the present disclosure. In some embodiments, an internucleotidic linkage is a phosphotriester linkage, phosphorothioate linkage (or phosphorothioate diester linkage, —OP(═O)(SH)O—, which as appreciated by those skilled in the art may exist as a salt form), or phosphorothioate triester linkage. In some embodiments, a modified internucleotidic linkage is a phosphorothioate linkage. In some embodiments, an internucleotidic linkage is one of, e.g., PNA (peptide nucleic acid) or PMO (phosphorodiamidate Morpholino oligomer) linkage. In some embodiments, a modified internucleotidic linkage is a non-negatively charged internucleotidic linkage. In some embodiments, a modified internucleotidic linkage is a neutral internucleotidic linkage (e.g., n001 in certain provided oligonucleotides). It is understood by a person of ordinary skill in the art that an internucleotidic linkage may exist as an anion or cation at a given pH due to the existence of acid or base moieties in the linkage. In some embodiments, a modified internucleotidic linkages is a modified internucleotidic linkages designated as s, s1, s2, s3, s4, s5, s6, s7, s8, s9, s10, s11, s12, s13, s14, s15, s16, s17 and s18 as described in WO 2017/210647.

In vitro: As used herein, the term “in vitro” refers to events that occur in an artificial environment, e.g., in a test tube or reaction vessel, in cell culture, etc., rather than within an organism (e.g., animal, plant and/or microbe).

In vivo: As used herein, the term “in vivo” refers to events that occur within an organism (e.g., animal, plant and/or microbe).

Linkage phosphorus: as defined herein, the phrase “linkage phosphorus” is used to indicate that the particular phosphorus atom being referred to is the phosphorus atom present in the internucleotidic linkage, which phosphorus atom corresponds to the phosphorus atom of a phosphodiester internucleotidic linkage as occurs in naturally occurring DNA and RNA. In some embodiments, a linkage phosphorus atom is in a modified internucleotidic linkage, wherein each oxygen atom of a phosphodiester linkage is optionally and independently replaced by an organic or inorganic moiety. In some embodiments, a linkage phosphorus atom is the P of Formula I as described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612). In some embodiments, a linkage phosphorus atom is chiral. In some embodiments, a linkage phosphorus atom is achiral (e.g., as in natural phosphate linkages).

Linker: The terms “linker”, “linking moiety” and the like refer to any chemical moiety which connects one chemical moiety to another. As appreciated by those skilled in the art, a linker can be bivalent or trivalent or more, depending on the number of chemical moieties the linker connects. In some embodiments, a linker is a moiety which connects one oligonucleotide to another oligonucleotide in a multimer. In some embodiments, a linker is a moiety optionally positioned between the terminal nucleoside and the solid support or between the terminal nucleoside and another nucleoside, nucleotide, or nucleic acid. In some embodiments, in an oligonucleotide a linker connects a chemical moiety (e.g., a targeting moiety, a lipid moiety, a carbohydrate moiety, etc.) with an oligonucleotide chain (e.g., through its 5′-end, 3′-end, nucleobase, sugar, internucleotidic linkage, etc.)

Modified nucleobase: The terms “modified nucleobase”, “modified base” and the like refer to a chemical moiety which is chemically distinct from a nucleobase, but which is capable of performing at least one function of a nucleobase. In some embodiments, a modified nucleobase is a nucleobase which comprises a modification. In some embodiments, a modified nucleobase is capable of at least one function of a nucleobase, e.g., forming a moiety in a polymer capable of base-pairing to a nucleic acid comprising an at least complementary sequence of bases. In some embodiments, a modified nucleobase is substituted A, T, C, G, or U, or a substituted tautomer of A, T, C, G, or U. In some embodiments, a modified nucleobase in the context of oligonucleotides refer to a nucleobase that is not A, T, C, G or U.

Modified nucleoside: The term “modified nucleoside” refers to a moiety derived from or chemically similar to a natural nucleoside, but which comprises a chemical modification which differentiates it from a natural nucleoside. Non-limiting examples of modified nucleosides include those which comprise a modification at the base and/or the sugar. Non-limiting examples of modified nucleosides include those with a 2′ modification at a sugar. Non-limiting examples of modified nucleosides also include abasic nucleosides (which lack a nucleobase). In some embodiments, a modified nucleoside is capable of at least one function of a nucleoside, e.g., forming a moiety in a polymer capable of base-pairing to a nucleic acid comprising an at least complementary sequence of bases.

Modified nucleotide: The term “modified nucleotide” includes any chemical moiety which differs structurally from a natural nucleotide but is capable of performing at least one function of a natural nucleotide. In some embodiments, a modified nucleotide comprises a modification at a sugar, base and/or internucleotidic linkage. In some embodiments, a modified nucleotide comprises a modified sugar, modified nucleobase and/or modified internucleotidic linkage. In some embodiments, a modified nucleotide is capable of at least one function of a nucleotide, e.g., forming a subunit in a polymer capable of base-pairing to a nucleic acid comprising an at least complementary sequence of bases.

Modified sugar: The term “modified sugar” refers to a moiety that can replace a sugar. A modified sugar mimics the spatial arrangement, electronic properties, or some other physicochemical property of a sugar. In some embodiments, as described in the present disclosure, a modified sugar is substituted ribose or deoxyribose. In some embodiments, a modified sugar comprises a 2′-modification. Examples of useful 2′-modification are widely utilized in the art and described herein. In some embodiments, a 2′-modification is 2′-OR, wherein R is optionally substituted C₁₋₁₀ aliphatic. In some embodiments, a 2′-modification is 2′-OMe. In some embodiments, a 2′-modification is 2′-MOE. In some embodiments, a modified sugar is a bicyclic sugar (e.g., a sugar used in LNA, BNA, etc.). In some embodiments, in the context of oligonucleotides, a modified sugar is a sugar that is not ribose or deoxyribose as typically found in natural RNA or DNA.

Nucleic acid: The term “nucleic acid”, as used herein, includes any nucleotides and polymers thereof. The term “polynucleotide”, as used herein, refers to a polymeric form of nucleotides of any length, either ribonucleotides (RNA) or deoxyribonucleotides (DNA) or a combination thereof. These terms refer to the primary structure of the molecules and, thus, include double- and single-stranded DNA, and double- and single-stranded RNA. These terms include, as equivalents, analogs of either RNA or DNA comprising modified nucleotides and/or modified polynucleotides, such as, though not limited to, methylated, protected and/or capped nucleotides or polynucleotides. The terms encompass poly- or oligo-ribonucleotides (RNA) and poly- or oligo-deoxyribonucleotides (DNA); RNA or DNA derived from N-glycosides or C-glycosides of nucleobases and/or modified nucleobases; nucleic acids derived from sugars and/or modified sugars; and nucleic acids derived from phosphate bridges and/or modified internucleotidic linkages. The term encompasses nucleic acids containing any combinations of nucleobases, modified nucleobases, sugars, modified sugars, phosphate bridges or modified internucleotidic linkages. Examples include, and are not limited to, nucleic acids containing ribose moieties, nucleic acids containing deoxy-ribose moieties, nucleic acids containing both ribose and deoxyribose moieties, nucleic acids containing ribose and modified ribose moieties. Unless otherwise specified, the prefix poly- refers to a nucleic acid containing 2 to about 10,000 nucleotide monomer units and wherein the prefix oligo- refers to a nucleic acid containing 2 to about 200 nucleotide monomer units.

Nucleobase: The term “nucleobase” refers to the parts of nucleic acids that are involved in the hydrogen-bonding that binds one nucleic acid strand to another complementary strand in a sequence specific manner. The most common naturally-occurring nucleobases are adenine (A), guanine (G), uracil (U), cytosine (C), and thymine (T). In some embodiments, a naturally-occurring nucleobases are modified adenine, guanine, uracil, cytosine, or thymine. In some embodiments, a naturally-occurring nucleobases are methylated adenine, guanine, uracil, cytosine, or thymine. In some embodiments, a nucleobase comprises a heteroaryl ring wherein a ring atom is nitrogen, and when in a nucleoside, the nitrogen is bonded to a sugar moiety. In some embodiments, a nucleobase comprises a heterocyclic ring wherein a ring atom is nitrogen, and when in a nucleoside, the nitrogen is bonded to a sugar moiety. In some embodiments, a nucleobase is a “modified nucleobase,” a nucleobase other than adenine (A), guanine (G), uracil (U), cytosine (C), and thymine (T). In some embodiments, a modified nucleobase is substituted A, T, C, G or U. In some embodiments, a modified nucleobase is a substituted tautomer of A, T, C, G, or U. In some embodiments, a modified nucleobases is methylated adenine, guanine, uracil, cytosine, or thymine. In some embodiments, a modified nucleobase mimics the spatial arrangement, electronic properties, or some other physicochemical property of the nucleobase and retains the property of hydrogen-bonding that binds one nucleic acid strand to another in a sequence specific manner. In some embodiments, a modified nucleobase can pair with all of the five naturally occurring bases (uracil, thymine, adenine, cytosine, or guanine) without substantially affecting the melting behavior, recognition by intracellular enzymes or activity of the oligonucleotide duplex. As used herein, the term “nucleobase” also encompasses structural analogs used in lieu of natural or naturally-occurring nucleotides, such as modified nucleobases and nucleobase analogs. In some embodiments, a nucleobase is optionally substituted A, T, C, G, or U, or an optionally substituted tautomer of A, T, C, G, or U. In some embodiments, a “nucleobase” refers to a nucleobase unit in an oligonucleotide or a nucleic acid (e.g., A, T, C, G or U as in an oligonucleotide or a nucleic acid).

Nucleoside: The term “nucleoside” refers to a moiety wherein a nucleobase or a modified nucleobase is covalently bound to a sugar or a modified sugar. In some embodiments, a nucleoside is a natural nucleoside, e.g., adenosine, deoxyadenosine, guanosine, deoxyguanosine, thymidine, uridine, cytidine, or deoxycytidine. In some embodiments, a nucleoside is a modified nucleoside, e.g., a substituted natural nucleoside selected from adenosine, deoxyadenosine, guanosine, deoxyguanosine, thymidine, uridine, cytidine, and deoxycytidine. In some embodiments, a nucleoside is a modified nucleoside, e.g., a substituted tautomer of a natural nucleoside selected from adenosine, deoxyadenosine, guanosine, deoxyguanosine, thymidine, uridine, cytidine, and deoxycytidine. In some embodiments, a “nucleoside” refers to a nucleoside unit in an oligonucleotide or a nucleic acid.

Nucleoside analog: The term “nucleoside analog” refers to a chemical moiety which is chemically distinct from a natural nucleoside, but which is capable of performing at least one function of a nucleoside. In some embodiments, a nucleoside analog comprises an analog of a sugar and/or an analog of a nucleobase. In some embodiments, a modified nucleoside is capable of at least one function of a nucleoside, e.g., forming a moiety in a polymer capable of base-pairing to a nucleic acid comprising a complementary sequence of bases.

Nucleotide: The term “nucleotide” as used herein refers to a monomeric unit of a polynucleotide that consists of a nucleobase, a sugar, and one or more internucleotidic linkages (e.g., phosphate linkages in natural DNA and RNA). The naturally occurring bases [guanine, (G), adenine, (A), cytosine, (C), thymine, (T), and uracil (U)] are derivatives of purine or pyrimidine, though it should be understood that naturally and non-naturally occurring base analogs are also included. The naturally occurring sugar is the pentose (five-carbon sugar) deoxyribose (which forms DNA) or ribose (which forms RNA), though it should be understood that naturally and non-naturally occurring sugar analogs are also included. Nucleotides are linked via internucleotidic linkages to form nucleic acids, or polynucleotides. Many internucleotidic linkages are known in the art (such as, though not limited to, phosphate, phosphorothioates, boranophosphates and the like). Artificial nucleic acids include PNAs (peptide nucleic acids), phosphotriesters, phosphorothionates, H-phosphonates, phosphoramidates, boranophosphates, methylphosphonates, phosphonoacetates, thiophosphonoacetates and other variants of the phosphate backbone of native nucleic acids, such as those described herein. In some embodiments, a natural nucleotide comprises a naturally occurring base, sugar and internucleotidic linkage. As used herein, the term “nucleotide” also encompasses structural analogs used in lieu of natural or naturally-occurring nucleotides, such as modified nucleotides and nucleotide analogs. In some embodiments, a “nucleotide” refers to a nucleotide unit in an oligonucleotide or a nucleic acid.

Oligonucleotide: The term “oligonucleotide” refers to a polymer or oligomer of nucleotides, and may contain any combination of natural and non-natural nucleobases, sugars, and internucleotidic linkages.

Oligonucleotides can be single-stranded or double-stranded. A single-stranded oligonucleotide can have double-stranded regions (formed by two portions of the single-stranded oligonucleotide) and a double-stranded oligonucleotide, which comprises two oligonucleotide chains, can have single-stranded regions for example, at regions where the two oligonucleotide chains are not complementary to each other. Example oligonucleotides include, but are not limited to structural genes, genes including control and termination regions, self-replicating systems such as viral or plasmid DNA, single-stranded and double-stranded RNAi agents and other RNA interference reagents (RNAi agents or iRNA agents), shRNA, antisense oligonucleotides, ribozymes, microRNAs, microRNA mimics, supermirs, aptamers, antimirs, antagomirs, Ul adaptors, triplex-forming oligonucleotides, G-quadruplex oligonucleotides, RNA activators, immuno-stimulatory oligonucleotides, and decoy oligonucleotides.

Oligonucleotides of the present disclosure can be of various lengths. In particular embodiments, oligonucleotides can range from about 2 to about 200 nucleosides in length. In various related embodiments, oligonucleotides, single-stranded, double-stranded, or triple-stranded, can range in length from about 4 to about 10 nucleosides, from about 10 to about 50 nucleosides, from about 20 to about 50 nucleosides, from about 15 to about 30 nucleosides, from about 20 to about 30 nucleosides in length. In some embodiments, the oligonucleotide is from about 9 to about 39 nucleosides in length. In some embodiments, the oligonucleotide is at least 4, 5, 6, 7, 8, 9, 10, 11, 12, 13, 14, 15, 16, 17, 18, 19, 20, 21, 22, 23, 24, or 25 nucleosides in length. In some embodiments, the oligonucleotide is at least 4 nucleosides in length. In some embodiments, the oligonucleotide is at least 5 nucleosides in length. In some embodiments, the oligonucleotide is at least 6 nucleosides in length. In some embodiments, the oligonucleotide is at least 7 nucleosides in length. In some embodiments, the oligonucleotide is at least 8 nucleosides in length. In some embodiments, the oligonucleotide is at least 9 nucleosides in length. In some embodiments, the oligonucleotide is at least 10 nucleosides in length. In some embodiments, the oligonucleotide is at least 11 nucleosides in length. In some embodiments, the oligonucleotide is at least 12 nucleosides in length. In some embodiments, the oligonucleotide is at least 15 nucleosides in length. In some embodiments, the oligonucleotide is at least 15 nucleosides in length. In some embodiments, the oligonucleotide is at least 16 nucleosides in length. In some embodiments, the oligonucleotide is at least 17 nucleosides in length. In some embodiments, the oligonucleotide is at least 18 nucleosides in length. In some embodiments, the oligonucleotide is at least 19 nucleosides in length. In some embodiments, the oligonucleotide is at least 20 nucleosides in length. In some embodiments, the oligonucleotide is at least 25 nucleosides in length. In some embodiments, the oligonucleotide is at least 30 nucleosides in length. In some embodiments, the oligonucleotide is a duplex of complementary strands of at least 18 nucleosides in length. In some embodiments, the oligonucleotide is a duplex of complementary strands of at least 21 nucleosides in length. In some embodiments, each nucleoside counted in an oligonucleotide length independently comprises A, T, C, G, or U, or optionally substituted A, T, C, G, or U, or an optionally substituted tautomer of A, T, C, G or U.

Oligonucleotide type: As used herein, the phrase “oligonucleotide type” is used to define an oligonucleotide that has a particular base sequence, pattern of backbone linkages (i.e., pattern of internucleotidic linkage types, for example, phosphate, phosphorothioate, phosphorothioate triester, etc.), pattern of backbone chiral centers [i.e., pattern of linkage phosphorus stereochemistry (Rp/Sp)], and pattern of backbone phosphorus modifications (e.g., pattern of “-XLR¹” groups in Formula I as described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612). In some embodiments, oligonucleotides of a common designated “type” are structurally identical to one another.

One of skill in the art will appreciate that synthetic methods of the present disclosure provide for a degree of control during the synthesis of an oligonucleotide strand such that each nucleotide unit of the oligonucleotide strand can be designed and/or selected in advance to have a particular stereochemistry at the linkage phosphorus and/or a particular modification at the linkage phosphorus, and/or a particular base, and/or a particular sugar. In some embodiments, an oligonucleotide strand is designed and/or selected in advance to have a particular combination of stereocenters at the linkage phosphorus. In some embodiments, an oligonucleotide strand is designed and/or determined to have a particular combination of modifications at the linkage phosphorus. In some embodiments, an oligonucleotide strand is designed and/or selected to have a particular combination of bases. In some embodiments, an oligonucleotide strand is designed and/or selected to have a particular combination of one or more of the above structural characteristics. In some embodiments, the present disclosure provides compositions comprising or consisting of a plurality of oligonucleotide molecules (e.g., chirally controlled oligonucleotide compositions). In some embodiments, all such molecules are of the same type (i.e., are structurally identical to one another). In some embodiments, however, provided compositions comprise a plurality of oligonucleotides of different types, typically in pre-determined relative amounts.

Optionally Substituted: As described herein, compounds, e.g., oligonucleotides, of the disclosure may contain optionally substituted and/or substituted moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. In some embodiments, an optionally substituted group is unsubstituted. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein. Certain substituents are described below.

Suitable monovalent substituents on a substitutable atom, e.g., a suitable carbon atom, are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, —SC(S)SR^{∘}; —(CH₂)₀₋₄SC(O)R^{∘}; —(CH₂)₀₋₄C(O)NR⁰²; —C(S)NR^{∘}₂; —C(S)SR^{∘}; —(CH₂)₀₋₄OC(O)NR⁰²; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —Si(R^{∘})₃; —OSi(R^{∘})₃; —B(R^{∘})₂; —OB(R^{∘})₂; —OB(OR^{∘})₂; —P(R^{∘})₂; —P(OR^{∘})₂; —P(R^{∘})(OR^{∘}); —OP(R^{∘})₂; —OP(OR^{∘})₂; —OP(R^{∘})(OR^{∘}); —P(O)(R^{∘})₂; —P(O)(OR^{∘})₂; —OP(O)(R^{∘})₂; —OP(O)(OR^{∘})₂; —OP(O)(OR^{∘})(SR^{∘}); —SP(O)(R^{∘})₂; —SP(O)(OR^{∘})₂; —N(R^{∘})P(O)(R^{∘})₂; —N(R^{∘})P(O)(OR^{∘})₂; —P(R^{∘})₂[B(R^{∘})₃]; —P(OR^{∘})₂[B(R^{∘})₃]; —OP(R^{∘})₂[B(R^{∘})₃]; —OP(OR^{∘})₂[B(R^{∘})₃]; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined herein and is independently hydrogen, C₁₋₂₀ aliphatic, C₁₋₂₀ heteroaliphatic having 1-5 heteroatoms independently selected from nitrogen, oxygen, sulfur, silicon and phosphorus, —CH₂—(C₆₋₁₄ aryl), —O(CH₂)₀₋₁(C₆₋₁₄ aryl), —CH₂-(5-14 membered heteroaryl ring), a 5-20 membered, monocyclic, bicyclic, or polycyclic, saturated, partially unsaturated or aryl ring having 0-5 heteroatoms independently selected from nitrogen, oxygen, sulfur, silicon and phosphorus, or, notwithstanding the definition above, two independent occurrences of R^{∘}, taken together with their intervening atom(s), form a 5-20 membered, monocyclic, bicyclic, or polycyclic, saturated, partially unsaturated or aryl ring having 0-5 heteroatoms independently selected from nitrogen, oxygen, sulfur, silicon and phosphorus, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, and a 5-6-membered saturated, partially unsaturated, or aryl ring having 0-4 heteroatoms independently selected from nitrogen, oxygen, and sulfur. Suitable divalent substituents on a saturated carbon atom of R^{◯} include ═O and ═S.

Suitable divalent substituents, e.g., on a suitable carbon atom, are independently the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, and an unsubstituted 5-6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, and an unsubstituted 5-6-membered saturated, partially unsaturated, and aryl ring having 0-4 heteroatoms independently selected from nitrogen, oxygen, and sulfur.

Suitable substituents on the aliphatic group of R* are independently halogen, —R^{•}, -(haloR^{•}), —OH, —OR^{•}, —O(haloR^{•}), —CN, —C(O)OH, —C(O)OR^{•}, —NH₂, —NHR^{•}, —NR′₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

In some embodiments, suitable substituents on a substitutable nitrogen are independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, and sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, and sulfur.

Suitable substituents on the aliphatic group of R^{\} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR′₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

Oral: The phrases “oral administration” and “administered orally” as used herein have their art-understood meaning referring to administration by mouth of a compound or composition.

P-modification: as used herein, the term “P-modification” refers to any modification at the linkage phosphorus other than a stereochemical modification. In some embodiments, a P-modification comprises addition, substitution, or removal of a pendant moiety covalently attached to a linkage phosphorus. In some embodiments, the “P-modification” is —X-L-R¹ wherein each of X, L and R′ is independently as defined and described in the present disclosure.

Parenteral: The phrases “parenteral administration” and “administered parenterally” as used herein have their art-understood meaning referring to modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal, and intrasternal injection and infusion.

Partially unsaturated: As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

Pharmaceutical composition: As used herein, the term “pharmaceutical composition” refers to an active agent, formulated together with one or more pharmaceutically acceptable carriers. In some embodiments, an active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, pharmaceutically acceptable salt include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, a provided compound comprises one or more acidic groups, e.g., an oligonucleotide, and a pharmaceutically acceptable salt is an alkali, alkaline earth metal, or ammonium (e.g., an ammonium salt of N(R)₃, wherein each R is independently defined and described in the present disclosure) salt. Representative alkali or alkaline earth metal salts include sodium, lithium, potassium, calcium, magnesium, and the like. In some embodiments, a pharmaceutically acceptable salt is a sodium salt. In some embodiments, a pharmaceutically acceptable salt is a potassium salt. In some embodiments, a pharmaceutically acceptable salt is a calcium salt. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate. In some embodiments, a provided compound comprises more than one acid groups, for example, an oligonucleotide may comprise two or more acidic groups (e.g., in natural phosphate linkages and/or modified internucleotidic linkages). In some embodiments, a pharmaceutically acceptable salt, or generally a salt, of such a compound comprises two or more cations, which can be the same or different. In some embodiments, in a pharmaceutically acceptable salt (or generally, a salt), all ionizable hydrogen (e.g., in an aqueous solution with a pKa no more than about 11, 10, 9, 8, 7, 6, 5, 4, 3, or 2; in some embodiments, no more than about 7; in some embodiments, no more than about 6; in some embodiments, no more than about 5; in some embodiments, no more than about 4; in some embodiments, no more than about 3) in the acidic groups are replaced with cations. In some embodiments, each phosphorothioate and phosphate group independently exists in its salt form (e.g., if sodium salt, —O—P(O)(SNa)—O— and —O—P(O)(ONa)—O—, respectively). In some embodiments, each phosphorothioate and phosphate internucleotidic linkage independently exists in its salt form (e.g., if sodium salt, —O—P(O)(SNa)—O— and —O—P(O)(ONa)—O—, respectively). In some embodiments, a pharmaceutically acceptable salt is a sodium salt of an oligonucleotide. In some embodiments, a pharmaceutically acceptable salt is a sodium salt of an oligonucleotide, wherein each acidic phosphate and modified phosphate group (e.g., phosphorothioate, phosphate, etc.), if any, exists as a salt form (all sodium salt).

Predetermined: By predetermined (or pre-determined) is meant deliberately selected or non-random or controlled, for example as opposed to randomly occurring, random, or achieved without control. Those of ordinary skill in the art, reading the present specification, will appreciate that the present disclosure provides technologies that permit selection of particular chemistry and/or stereochemistry features to be incorporated into oligonucleotide compositions, and further permits controlled preparation of oligonucleotide compositions having such chemistry and/or stereochemistry features. Such provided compositions are “predetermined” as described herein. Compositions that may contain certain oligonucleotides because they happen to have been generated through a process that are not controlled to intentionally generate the particular chemistry and/or stereochemistry features are not “predetermined” compositions. In some embodiments, a predetermined composition is one that can be intentionally reproduced (e.g., through repetition of a controlled process). In some embodiments, a predetermined level of a plurality of oligonucleotides in a composition means that the absolute amount, and/or the relative amount (ratio, percentage, etc.) of the plurality of oligonucleotides in the composition is controlled. In some embodiments, a predetermined level of a plurality of oligonucleotides in a composition is achieved through chirally controlled oligonucleotide preparation.

Protecting group: The term “protecting group,” as used herein, is well known in the art and includes those described in detail in Protecting Groups in Organic Synthesis, T. W. Greene and P. G. M. Wuts, 3^{rd} edition, John Wiley & Sons, 1999, the entirety of which is incorporated herein by reference. Also included are those protecting groups specially adapted for nucleoside and nucleotide chemistry described in Current Protocols in Nucleic Acid Chemistry, edited by Serge L. Beaucage et al. 06/2012, the entirety of Chapter 2 is incorporated herein by reference. Suitable amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), (3-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Suitably protected carboxylic acids further include, but are not limited to, silyl-, alkyl-, alkenyl-, aryl-, and arylalkyl-protected carboxylic acids. Examples of suitable silyl groups include trimethylsilyl, triethylsilyl, t-butyldimethylsilyl, t-butyldiphenylsilyl, triisopropylsilyl, and the like. Examples of suitable alkyl groups include methyl, benzyl, p-methoxybenzyl, 3,4-dimethoxybenzyl, trityl, t-butyl, tetrahydropyran-2-yl. Examples of suitable alkenyl groups include allyl. Examples of suitable aryl groups include optionally substituted phenyl, biphenyl, or naphthyl. Examples of suitable arylalkyl groups include optionally substituted benzyl (e.g., p-methoxybenzyl (MPM), 3,4-dimethoxybenzyl, O-nitrobenzyl, p-nitrobenzyl, p-halobenzyl, 2,6-dichlorobenzyl, p-cyanobenzyl), and 2- and 4-picolyl.

Suitable hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

In some embodiments, a hydroxyl protecting group is acetyl, t-butyl, tbutoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, benzoyl, p-phenylbenzoyl, 2,6-dichlorobenzyl, diphenylmethyl, p-nitrobenzyl, triphenylmethyl (trityl), 4,4′-dimethoxytrityl, trimethylsilyl, triethylsilyl, t-butyldimethylsilyl, t-butyldiphenylsilyl, triphenylsilyl, triisopropylsilyl, benzoylformate, chloroacetyl, trichloroacetyl, trifiuoroacetyl, pivaloyl, 9-fluorenylmethyl carbonate, mesylate, tosylate, triflate, trityl, monomethoxytrityl (MMTr), 4,4′-dimethoxytrityl, (DMTr) and 4,4′,4″-trimethoxytrityl (TMTr), 2-cyanoethyl (CE or Cne), 2-(trimethylsilyl)ethyl (TSE), 2-(2-nitrophenyl)ethyl, 2-(4-cyanophenyl)ethyl 2-(4-nitrophenyl)ethyl (NPE), 2-(4-nitrophenylsulfonyl)ethyl, 3,5-dichlorophenyl, 2,4-dimethylphenyl, 2-nitrophenyl, 4-nitrophenyl, 2,4,6-trimethylphenyl, 2-(2-nitrophenyl)ethyl, butylthiocarbonyl, 4,4′,4″-tris(benzoyloxy)trityl, diphenylcarbamoyl, levulinyl, 2-(dibromomethyl)benzoyl (Dbmb), 2-(isopropylthiomethoxymethyl)benzoyl (Ptmt), 9-phenylxanthen-9-yl (pixyl) or 9-(p-methoxyphenyl)xanthine-9-yl (MOX). In some embodiments, each of the hydroxyl protecting groups is, independently selected from acetyl, benzyl, t- butyldimethylsilyl, t-butyldiphenylsilyl and 4,4′-dimethoxytrityl. In some embodiments, the hydroxyl protecting group is selected from the group consisting of trityl, monomethoxytrityl and 4,4′-dimethoxytrityl group. In some embodiments, a phosphorous linkage protecting group is a group attached to the phosphorous linkage (e.g., an internucleotidic linkage) throughout oligonucleotide synthesis. In some embodiments, a protecting group is attached to a sulfur atom of an phosphorothioate group. In some embodiments, a protecting group is attached to an oxygen atom of an internucleotide phosphorothioate linkage. In some embodiments, a protecting group is attached to an oxygen atom of the internucleotide phosphate linkage. In some embodiments a protecting group is 2-cyanoethyl (CE or Cne), 2-trimethylsilylethyl, 2-nitroethyl, 2-sulfonylethyl, methyl, benzyl, o-nitrobenzyl, 2-(p-nitrophenyl)ethyl (NPE or Npe), 2-phenylethyl, 3-(N-tert-butylcarboxamido)-1-propyl, 4-oxopentyl, 4-methylthio-1-butyl, 2-cyano-1,1-dimethylethyl, 4-N-methylaminobutyl, 3-(2-pyridyl)-1-propyl, 2-[N-methyl-N-(2-pyridyl)]aminoethyl, 2-(N-formyl,N-methyl)aminoethyl, or 4-[N-methyl-N-(2,2,2-trifluoroacetyl)amino]butyl.

Subject: As used herein, the term “subject” or “test subject” refers to any organism to which a provided compound (e.g., a provided oligonucleotide) or composition is administered in accordance with the present disclosure e.g., for experimental, diagnostic, prophylactic and/or therapeutic purposes. Typical subjects include animals (e.g., mammals such as mice, rats, rabbits, non-human primates, and humans; insects; worms; etc.) and plants. In some embodiments, a subject is a human. In some embodiments, a subject may be suffering from and/or susceptible to a disease, disorder and/or condition.

Substantially: As used herein, the term “substantially” refers to the qualitative condition of exhibiting total or near-total extent or degree of a characteristic or property of interest. A base sequence which is substantially complementary to a second sequence is not identical to the second sequence, but is mostly or nearly identical to the second sequence. In addition, one of ordinary skill in the biological and/or chemical arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Sugar: The term “sugar” refers to a monosaccharide or polysaccharide in closed and/or open form. In some embodiments, sugars are monosaccharides. In some embodiments, sugars are polysaccharides. Sugars include, but are not limited to, ribose, deoxyribose, pentofuranose, pentopyranose, and hexopyranose moieties. As used herein, the term “sugar” also encompasses structural analogs used in lieu of conventional sugar molecules, such as glycol, polymer of which forms the backbone of the nucleic acid analog, glycol nucleic acid (“GNA”), etc. As used herein, the term “sugar” also encompasses structural analogs used in lieu of natural or naturally-occurring nucleotides, such as modified sugars and nucleotide sugars. In some embodiments, a sugar is a RNA or DNA sugar (ribose or deoxyribose). In some embodiments, a sugar is a modified ribose or deoxyribose sugar, e.g., 2′-modified, 5′-modified, etc. As described herein, in some embodiments, when used in oligonucleotides and/or nucleic acids, modified sugars may provide one or more desired properties, activities, etc. In some embodiments, a sugar is optionally substituted ribose or deoxyribose. In some embodiments, a “sugar” refers to a sugar unit in an oligonucleotide or a nucleic acid.

Susceptible to: An individual who is “susceptible to” a disease, disorder and/or condition is one who has a higher risk of developing the disease, disorder and/or condition than does a member of the general public. In some embodiments, an individual who is susceptible to a disease, disorder and/or condition is predisposed to have that disease, disorder and/or condition. In some embodiments, an individual who is susceptible to a disease, disorder and/or condition may not have been diagnosed with the disease, disorder and/or condition. In some embodiments, an individual who is susceptible to a disease, disorder and/or condition may exhibit symptoms of the disease, disorder and/or condition. In some embodiments, an individual who is susceptible to a disease, disorder and/or condition may not exhibit symptoms of the disease, disorder and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutic agent: As used herein, the term “therapeutic agent” in general refers to any agent that elicits a desired effect (e.g., a desired biological, clinical, or pharmacological effect) when administered to a subject. In some embodiments, an agent is considered to be a therapeutic agent if it demonstrates a statistically significant effect across an appropriate population. In some embodiments, an appropriate population is a population of subjects suffering from and/or susceptible to a disease, disorder or condition. In some embodiments, an appropriate population is a population of model organisms. In some embodiments, an appropriate population may be defined by one or more criterion such as age group, gender, genetic background, preexisting clinical conditions, prior exposure to therapy. In some embodiments, a therapeutic agent is a substance that alleviates, ameliorates, relieves, inhibits, prevents, delays onset of, reduces severity of, and/or reduces incidence of one or more symptoms or features of a disease, disorder, and/or condition in a subject when administered to the subject in an effective amount. In some embodiments, a “therapeutic agent” is an agent that has been or is required to be approved by a government agency before it can be marketed for administration to humans. In some embodiments, a “therapeutic agent” is an agent for which a medical prescription is required for administration to humans. In some embodiments, a therapeutic agent is a provided compound, e.g., a provided oligonucleotide.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Unimer: the term “unimer,” as used herein, refers to an oligonucleotide whose pattern of structural features characterizing each individual nucleotide unit is such that all nucleotide units within the oligonucleotide share at least one common structural feature, e.g., at the internucleotidic phosphorus linkage. In some embodiments, a common structural feature is common stereochemistry at the linkage phosphorus or a common modification at the linkage phosphorus. In some embodiments, an oligonucleotide is a unimer.

In some embodiments, a unimer is a “stereounimer,” e.g., all internucleotidic linkages have the same stereochemistry at the linkage phosphorus.

In some embodiments, a unimer is a “P-modification unimer”, e.g., all internucleotidic linkages have the same modification at the linkage phosphorus.

In some embodiments, a unimer is a “linkage unimer,” e.g., all nucleotide internucleotidic linkages have the same stereochemistry and the same modifications at the linkage phosphorus.

In some embodiments, a unimer is a “sugar modification unimer,” e.g., all nucleoside units comprise the same sugar modification.

Unit dose: The expression “unit dose” as used herein refers to an amount administered as a single dose and/or in a physically discrete unit of a pharmaceutical composition. In many embodiments, a unit dose contains a predetermined quantity of an active agent. In some embodiments, a unit dose contains an entire single dose of the agent. In some embodiments, more than one unit dose is administered to achieve a total single dose. In some embodiments, administration of multiple unit doses is required, or expected to be required, in order to achieve an intended effect. A unit dose may be, for example, a volume of liquid (e.g., an acceptable carrier) containing a predetermined quantity of one or more therapeutic agents, a predetermined amount of one or more therapeutic agents in solid form, a sustained release formulation or drug delivery device containing a predetermined amount of one or more therapeutic agents, etc. It will be appreciated that a unit dose may be present in a formulation that includes any of a variety of components in addition to the therapeutic agent(s). For example, acceptable carriers (e.g., pharmaceutically acceptable carriers), diluents, stabilizers, buffers, preservatives, etc., may be included as described infra. It will be appreciated by those skilled in the art, in many embodiments, a total appropriate daily dosage of a particular therapeutic agent may comprise a portion, or a plurality, of unit doses, and may be decided, for example, by the attending physician within the scope of sound medical judgment. In some embodiments, the specific effective dose level for any particular subject or organism may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts.

Unsaturated: The term “unsaturated,” as used herein, means that a moiety has one or more units of unsaturation.

Wild-type: As used herein, the term “wild-type” has its art-understood meaning that refers to an entity having a structure and/or activity as found in nature in a “normal” (as contrasted with mutant, diseased, altered, etc.) state or context. Those of ordinary skill in the art will appreciate that wild type genes and polypeptides often exist in multiple different forms (e.g., alleles).

As those skilled in the art will appreciate, methods and compositions described herein relating to provided compounds (e.g., oligonucleotides) generally also apply to pharmaceutically acceptable salts of such compounds.

### DESCRIPTION OF CERTAIN EMBODIMENTS

Oligonucleotides are useful tools for a wide variety of applications. For example, USH2A oligonucleotides are useful in therapeutic, diagnostic, and research applications, including the treatment of a variety of USH2A-related conditions, disorders, and diseases, including Usher Syndrome (e.g., Usher Syndrome Type 2A), atypical Usher syndrome, and nonsyndromic retinitis pigmentosa. The use of naturally occurring nucleic acids (e.g., unmodified DNA or RNA) is limited, for example, by their susceptibility to endo- and exo-nucleases. As such, various synthetic counterparts have been developed to circumvent these shortcomings and/or to further improve various properties and activities. These include synthetic oligonucleotides that contain chemical modifications, e.g., base modifications, sugar modifications, backbone modifications, etc., which, among other things, render these molecules less susceptible to degradation and improve other properties and/or activities. From a structural point of view, modifications to internucleotidic linkages can introduce chirality, and certain properties may be affected by configurations of linkage phosphorus atoms of oligonucleotides. For example, binding affinity, sequence specific binding to complementary RNA, stability to nucleases, cleavage of target nucleic acids, delivery, pharmacokinetics, etc. can be affected by, inter alia, chirality of backbone linkage phosphorus atoms. Among other things, the present disclosure utilizes technologies for controlling various structural elements, e.g., sugar modifications and patterns thereof, nucleobase modifications and patterns thereof, modified internucleotidic linkages and patterns thereof, linkage phosphorus stereochemistry and patterns thereof, additional chemical moieties (moieties that are not typically in an oligonucleotide chain) and patterns thereof, etc. With the capability to fully control structural elements of oligonucleotides, the present disclosure provides oligonucleotides with improved and/or new properties and/or activities for various applications, e.g., as therapeutic agents, probes, etc. For example, as demonstrated herein, provided oligonucleotides and compositions thereof are particularly powerful for reducing levels of transcripts (and products (e.g., proteins) encoded thereby) associated with various conditions, disorders or diseases, e.g., transcripts comprising one or more mutations in exon 13 of USH2A), and/or provide increased levels of transcripts with skipped exons (e.g., exon 13 of USH2A which comprises one or more mutations associated with conditions, disorders or diseases) which transcripts encode products (e.g., proteins) that have increased levels of one or more desirable functions compared to the corresponding transcripts without exon skipping.

In some embodiments, provided oligonucleotides target an USH2A gene transcript, and can reduce levels of mutant USH2A transcripts which comprise one or more mutations associated with a condition, disorder or disease (e.g., one or more mutations in exon 13 associated with Usher Syndrome (e.g., Usher Syndrome Type 2A), atypical Usher syndrome, nonsyndromic retinitis pigmentosa, etc.) and/or one or more products encoded thereby (e.g., a mutant USH2A protein comprising a mutation corresponding to a mutation in exon 13), by skipping of a deleterious exon in the USH2A transcript, and increase levels of an USH2A transcript with a deleterious exon skipped and/or a product encoded thereby (e.g, an internally truncated protein capable of mediating at least one function of USH2A at a level higher than the protein produced from corresponding transcripts without exon skipping). In some embodiments, a deleterious exon is exon 13 (Ex. 13). Such oligonucleotides are particularly useful for preventing and/or treating USH2A-related conditions, disorders and/or diseases, including Usher Syndrome (e.g., Usher Syndrome Type 2A), atypical Usher syndrome, and nonsyndromic retinitis pigmentosa.

In some embodiments, such oligonucleotides are designed to address the underlying cause of the vision loss associated with USH2A-related conditions, disorders and/or diseases, including Usher Syndrome (e.g., Usher Syndrome Type 2A), atypical Usher syndrome, and nonsyndromic retinitis pigmentosa, e.g., due to mutations in exon 13 of the USH2A gene. In some embodiments, such oligonucleotides are designed to address the underlying cause of deafness associated with USH2A-related conditions, disorders and/or diseases, including Usher Syndrome (e.g., Usher Syndrome Type 2A), atypical Usher syndrome, and nonsyndromic retinitis pigmentosa, e.g., due to mutations in exon 13 of the USH2A gene.

In some embodiments, an USH2A oligonucleotide capable of mediating skipping of an exon (e.g., exon 13) in an USH2A gene transcript shows high specificity for skipping that exon and not others (e.g., an adjacent exon). In some embodiments, an USH2A oligonucleotide has a high specificity for skipping a particular USH2A exon (e.g., exon 13). In some embodiments, an USH2A oligonucleotide has a specificity for skipping a particular USH2A exon of at least about 2, at least about 2.3, at least about 2.5, at least about 2.7, at least about 3, at least about 3.3, at least about 3.3, at least about 3.5, at least about 3.7, at least about 4, at least about 4.3, at least about 4.5, at least about 4.7, or at least about 5 [calculated as a ratio of the level of skipping of a particular exon (such as exon 13) compared to the level of skipping of that exon and an adjacent exon]. Non-limiting examples of USH2A oligonucleotides which showed specificity in their ability to skip an exon (e.g., exon 13) of an USH2A transcript include but are not limited to: WV-2110, WV-21105, WV-20885, WV-20891, WV-20892, WV-20902, WV-20908, and WV-20988.

In some embodiments, an USH2A oligonucleotide comprises a sequence that is identical to or is completely or substantially complementary to 10, 11, 12, 13, 14, 15, 16, 17, 18, 19, 20, 21, 22, 23, 24, 25, typically 15, 16, 17, 18, 19, 20, 21, 22, 23, 24, 25 or more, contiguous bases of an USH2A genomic sequence or a transcript therefrom (e.g., pre-mRNA, mRNA, etc.). In some embodiments, an USH2A oligonucleotide comprises a sequence that is completely complementary to 10, 11, 12, 13, 14, 15, 16, 17, 18, 19, 20, 21, 22, 23, 24, 25 or more, typically 15, 16, 17, 18, 19, 20, 21, 22, 23, 24, 25 or more, contiguous bases of an USH2A gene transcript. In some embodiments, an oligonucleotide that targets USH2A can hybridize with an USH2A gene transcript and can mediate skipping of a deleterious exon in the gene transcript. In some embodiments, a gene transcript is also referenced as a transcript, and includes but is not limited to, a nucleic acid transcribed from a gene (e.g., a chromosomal gene), including but not limited to a pre-mRNA, RNA, unprocessed RNA, processed RNA, etc. Those skilled in the art will appreciate that a “USH2A oligonucleotide” may have a nucleotide sequence that is identical (or substantially identical) or complementary (or substantially complementary) to an USH2A base sequence (e.g., a genomic sequence, a transcript sequence, a mRNA sequence, etc.) or a portion thereof. In some embodiments, an USH2A oligonucleotide comprises a sequence that is identical to or is completely complementary to 10, 11, 12, 13, 14, 15, 16, 17, 18, 19, 20, 21, 22, 23, 24, 25, typically 15, 16, 17, 18, 19, 20, 21, 22, 23, 24, 25 or more, contiguous bases of an USH2A genomic sequence or a transcript therefrom. In some embodiments, an USH2A oligonucleotide comprises a sequence that is completely complementary to 10, 11, 12, 13, 14, 15, 16, 17, 18, 19, 20, 21, 22, 23, 24, 25 or more, typically 15, 16, 17, 18, 19, 20, 21, 22, 23, 24, 25 or more, contiguous bases of an USH2A transcript.

In some embodiments, the present disclosure provides an USH2A oligonucleotide wherein the oligonucleotide has a base sequence which is or comprises at least 10 contiguous bases of an USH2A sequence (e.g., a sequence of an USH2A gene, transcript, etc.) disclosed herein, or of a sequence that is complementary to an USH2A sequence disclosed herein, and wherein each T can be independently substituted with U and vice versa. In some embodiments, the present disclosure provides an USH2A oligonucleotide as disclosed herein, e.g., in a Table. In some embodiments, the present disclosure provides an USH2A oligonucleotide having a base sequence disclosed herein, e.g., in a Table, or a portion thereof comprising at least 10 contiguous bases, wherein the USH2A oligonucleotide is stereorandom or not chirally controlled, and wherein each T can be independently substituted with U and vice versa.

In some embodiments, internucleotidic linkages of an oligonucleotide comprise or consist of 1-5, 1-10, 1-15, 1-20, 1-25, 1-30, 1-40, 1-50, or 1, 2, 3, 4, 5, 6, 7, 8, 9, 10, 11, 12, 13, 14, 15, 16, 17, 18, 19, 20 or more chirally controlled internucleotidic linkages. In some embodiments, two or more chirally controlled internucleotidic linkages (e.g., 2-5, 2-10, 2-15, 2-20, 2-25, 2-30, 2-40, 2-50, or 2, 3, 4, 5, 6, 7, 8, 9, 10, 11, 12, 13, 14, 15, 16, 17, 18, 19, or 20 more) are consecutive. In some embodiments, an oligonucleotide composition of the present disclosure comprises oligonucleotides of the same constitution, wherein one or more (e.g., 1-5, 1-10, 1-15, 1-20, 1-25, 1-30, 1-40, 1-50, or 1, 2, 3, 4, 5, 6, 7, 8, 9, 10, 11, 12, 13, 14, 15, 16, 17, 18, 19, 20 or more) internucleotidic linkages are chirally controlled and one or more (e.g., 1-5, 1-10, 1-15, 1-20, 1-25, 1-30, 1-40, 1-50, or 1, 2, 3, 4, 5, 6, 7, 8, 9, 10, 11, 12, 13, 14, 15, 16, 17, 18, 19, 20 or more) internucleotidic linkages are stereorandom (not chirally controlled). In some embodiments, the present disclosure provides an USH2A oligonucleotide composition wherein the USH2A oligonucleotides comprise at least one chirally controlled internucleotidic linkage. In some embodiments, the present disclosure provides an USH2A oligonucleotide composition wherein the USH2A oligonucleotides are stereorandom or not chirally controlled. In some embodiments, in an USH2A oligonucleotide, at least one internucleotidic linkage is stereorandom and at least one internucleotidic linkage is chirally controlled.

In some embodiments, internucleotidic linkages of an oligonucleotide comprise or consist of one or more (e.g., 1-5, 1-10, 1-15, 1-20, 1-25, 1-30, 1-40, 1-50, or 1, 2, 3, 4, 5, 6, 7, 8, 9, 10, 11, 12, 13, 14, 15, 16, 17, 18, 19, 20 or more) negatively charged internucleotidic linkages (e.g., phosphorothioate internucleotidic linkages, natural phosphate linkages, etc.). In some embodiments, internucleotidic linkages of an oligonucleotide comprise or consist of one or more (e.g., 1-5, 1-10, 1-15, 1-20, 1-25, 1-30, 1-40, 1-50, or 1, 2, 3, 4, 5, 6, 7, 8, 9, 10, 11, 12, 13, 14, 15, 16, 17, 18, 19, 20 or more) negatively charged chiral internucleotidic linkages (e.g., phosphorothioate internucleotidic linkages).

In some embodiments, the present disclosure provides an USH2A oligonucleotide composition wherein the USH2A oligonucleotides comprise at least one chirally controlled internucleotidic linkage, and at least one non-negatively charged internucleotidic linkage.

In some embodiments, internucleotidic linkages of an oligonucleotide comprise or consist of one or more (e.g., 1-5, 1-10, 1-15, 1-20, 1-25, 1-30, 1-40, 1-50, or 1, 2, 3, 4, 5, 6, 7, 8, 9, 10, 11, 12, 13, 14, 15, 16, 17, 18, 19, 20 or more) non-negatively charged internucleotidic linkages. In some embodiments, internucleotidic linkages of an oligonucleotide comprise or consist of one or more (e.g., 1-5, 1-10, 1-15, 1-20, 1-25, 1-30, 1-40, 1-50, or 1, 2, 3, 4, 5, 6, 7, 8, 9, 10, 11, 12, 13, 14, 15, 16, 17, 18, 19, 20 or more) neutral chiral internucleotidic linkages. In some embodiments, the present disclosure pertains to an USH2A oligonucleotide which comprises at least one neutral or non-negatively charged internucleotidic linkage as described in the present disclosure.

In some embodiments, an USH2A oligonucleotide or oligonucleotide composition comprises the base sequence of (or a portion of at least 10 contiguous bases of the base sequence of) any USH2A oligonucleotide described herein, and/or any particular structure (e.g., a sugar or sugar modification, a nucleobase or modified nucleobase, or internucleotidic linkage or modified internucleotidic linkage, or any additional chemical moiety) described herein. In some embodiments, an USH2A oligonucleotide or oligonucleotide composition comprises the base sequence of (or a portion of at least 10 contiguous bases of the base sequence of) any USH2A oligonucleotide described herein, and/or any particular structure (e.g., a sugar or sugar modification, a nucleobase or modified nucleobase, or internucleotidic linkage or modified internucleotidic linkage, or any additional chemical moiety) described herein, wherein the oligonucleotide is capable of mediating skipping of a deleterious exon of an USH2A gene transcript. In some embodiments, an USH2A oligonucleotide or oligonucleotide composition comprises the base sequence of (or a portion of at least 10 contiguous bases of the base sequence of) any USH2A oligonucleotide described herein, and/or any particular structure (e.g., a sugar or sugar modification, a nucleobase or modified nucleobase, or internucleotidic linkage or modified internucleotidic linkage, or any additional chemical moiety) described herein, wherein the oligonucleotide is capable of mediating skipping of a deleterious exon of an USH2A gene transcript, and is useful for treatment, amelioration or delay of onset of at least one symptom of an USH2A-related disease, disorder or condition, such as Usher Syndrome (e.g., Usher Syndrome Type 2A), atypical Usher syndrome, or nonsyndromic retinitis pigmentosa.

### USH2A

In some embodiments, USH2A refers to a wild-type or mutant gene, gene transcript or a gene product thereof (including but not limited to, a nucleic acid, including but not limited to a DNA or RNA, or a wild-type or mutant protein encoded thereby), or a variant or isoform thereof, from any species, a mutation in which is related to and/or associated with an USH2A-related disease, disorder or conditions (including but not limited to Usher Syndrome type IIa, atypical Usher syndrome, and nonsyndromic retinitis pigmentosa), and which may be known as: USH2A, RP39, US2, USH2, dJ1111A8.1, Usher syndrome 2A (autosomal recessive, mild), or usherin. Various USH2A sequences, including variants and isoforms thereof, from human, mouse, rat, monkey, etc., are readily available to those of skill in the art. In some embodiments, USH2A is a human or mouse USH2A, which is wild-type or mutant.

In some embodiments, an USH2A gene transcript includes a wild-type USH2A gene transcript, an USH2A gene transcript comprising a deleterious mutation(s) or deleterious exon(s), and an USH2A gene transcript in which a deleterious exon has been skipped. In some embodiments, a deleterious exon is an exon comprising a deleterious mutation, e.g., a mutation related to or associated with an USH2A-related disease, disorder or condition, including but not limited to Usher Syndrome, or Usher Syndrome Type IIA (2A), atypical Usher syndrome, or nonsyndromic retinitis pigmentosa. In some embodiments, an USH2A protein includes an USH2A protein variant translated from an USH2A gene transcript in which an exon has been skipped.

Without wishing to be bound by any particular theory, the present disclosure notes that various mutations (e.g., a disease-associated mutations) in USH2A are reportedly a key factor in USH2A-related diseases and disorders such as Usher syndrome type IIA (2A), atypical Usher syndrome, and nonsyndromic retinitis pigmentosa.

In some embodiments, provided oligonucleotides and compositions thereof are capable of providing an increase of the level of skipping of an exon in an USH2A gene transcript or a gene product thereof. In some embodiments, a provided oligonucleotide or composition targets an USH2A gene and is useful for treatment of USH2A-related conditions, disorders or diseases. In some embodiments, the present disclosure provides oligonucleotides and compositions for preventing and/or treating USH2A-related conditions, disorders or diseases. In some embodiments, the present disclosure provides methods for preventing and/or treating USH2A-related conditions, disorders or diseases, comprising administering to a subject susceptible thereto or suffering therefrom a therapeutically effective amount of a provided USH2A oligonucleotide or a composition thereof. USH2A-related conditions, disorders or diseases are extensively described in the art.

In some embodiments, an USH2A-related condition, disorder or disease is a condition, disorder or disease that is related to, caused by and/or associated with abnormal, reduced or excessive activity, level and/or expression, or abnormal tissue or inter- or intracellular distribution, of an USH2A gene transcript or a gene product thereof. In some embodiments, an USH2A-related condition, disorder or disease is associated with USH2A if the presence, level and/or form of transcription of an USH2A region, an USH2A gene transcript and/or a product encoded thereby correlates with incidence of and/or susceptibility to the condition, disorder or disease (e.g., across a relevant population). In some embodiments, an USH2A-related condition, disorder or disease is a condition, disorder or disease in which reduction of the level, expression and/or activity of a mutant version of, or in which increase of the level, expression and/or activity of a wild-type version of, an USH2A gene transcript or a product thereof ameliorates, prevents and/or reduces the severity of the condition, disorder or disease.

The Usher syndrome type IIA gene (USH2A) was reportedly identified on chromosome 1q41, and encodes a protein possessing 10 laminin epidermal growth factor and four fibronectin type 3 domains, both commonly observed in extracellular matrix proteins. Murine and rat orthologs of human USH2A reportedly exist. The mouse ortholog was reportedly mapped by fluorescence in situ hybridization to mouse chromosome 1 in the region syntenic to human chromosome 1q41. The rat ortholog has reportedly been localized by radiation hybrid mapping to rat chromosome 13 between d13rat49 and d13rat76. The mouse and rat genes, similar to human USH2A, are reportedly expressed in retina and cochlea. Mouse USH2A reportedly encodes a 161-kDa protein that shows 68% identity and 9% similarity to the human USH2A protein. Rat USH2A reportedly encodes a 167-kDa protein with 64% identity and 10% similarity to the human protein and 81% identity and 5% similarity to the mouse USH2A protein. The predicted amino acid sequence of the mouse and rat proteins, like their human counterpart, reportedly contains a leader sequence, an amino-terminal globular domain, 10 laminin epidermal growth factor domains, and four carboxy-terminal fibronectin type III motifs. With in situ hybridization, the cellular expression of the USH2A gene in rat, mouse, and human retinas was reportedly compared. USH2A mRNA in the adult rat, mouse, and human is reportedly expressed in the cells of the outer nuclear layer of the retina, one of the target tissues of the disease.

In some embodiments, USH2A is also referenced as: USH2A, USH2A, RP39, US2, USH2, dJ1111A8.1, Usher syndrome 2A (autosomal recessive, mild), usherin; mouse and rat orthologs: USH2A; External IDs: MGI: 1341292; HomoloGene: 66151; GeneCards: USH2A; Gene ontology: Orthologs: Species: Human; Entrez: 7399; Ensembl: ENSG00000042781; UniProt: 075445; RefSeq (mRNA): NM_206933; NM_007123; OMIM 608400; RefSeq (protein): NP_009054; NP_996816; Location (UCSC): Chr 1: 215.62-216.42 Mb; PubMed search: [3]; Gene ontology: Orthologs: Species Mouse; Entrez: 22283; Ensembl: ENSMUSG00000026609; UniProt: Q2QI47; RefSeq (mRNA): NM_021408; RefSeq (protein): NP_067383; Location (UCSC): Chr 1: 188.26-188.97 Mb.

In some embodiments, the present disclosure pertains to the use of an USH2A oligonucleotide in increasing the expression, level and/or activity of an alternatively spliced USH2A gene transcript (e.g., wherein a deleterious exon has been skipped) or a gene product thereof (e.g., increasing the level of an USH2A protein translated from an USH2A gene transcript in which a deleterious exon has been skipped, wherein the USH2A protein is internally truncated but capable of mediating at least one activity of USH2A).

In some embodiments, a mutant USH2A is designated mUSH2A, muUSH2A, m USH2A, mu USH2A, MU USH2A, or the like, wherein m or mu indicate mutant. In some embodiments, a wild type USH2A is designated wild-type USH2A, wtUSH2A, wt USH2A, WT USH2A, WTUSH2A, or the like, wherein wt indicates wild-type. In some embodiments, a mutant USH2A (or an USH2A variant) comprises a disease-associated mutation.

In some embodiments, a human USH2A is designated hUSH2A. In some embodiments, a mutant human USH2A is designated mUSH2A. In some embodiments, when a mouse is utilized, a mouse USH2A may be referred to as mUSH2A as those skilled in the art will appreciate in view of the context.

In some embodiments, a disease-associated (e.g., pathogenic) mutation is a mutation which is associated with a particular disease, disorder or condition (in the present disclosure, for example, an USH2A-related disease, disorder or condition). In some embodiments, a disease-associated mutation may be found in the genome of a patient suffering from or susceptible to a particular disease, disorder or condition (for example, an USH2A-related disease, disorder or condition), but is either absent or more rarely found in the genome of a patient who is not suffering from or susceptible to the disease, disorder or condition.

In some embodiments, in some patients of Usher Syndrome (e.g., Usher Syndrome Type 2A), the genome of the patient is lacking in a wild-type allele of USH2A and has only a mutant allele of USH2A (e.g., an allele comprising a deleterious mutation or a deleterious exon).

In some embodiments, an USH2A oligonucleotide is complementary to a portion of an USH2A nucleic acid sequence, e.g., an USH2A gene sequence, an USH2A transcript, an USH2A mRNA sequence, etc. In some embodiments, a portion is or comprises 10, 11, 12, 13, 14, 15, 16, 17, 18, 19, 20, 21, 22, 23, 24, 25, typically 15, 16, 17, 18, 19, 20, 21, 22, 23, 24, 25 or more contiguous nucleobases. In some embodiments, a portion is or comprises at least 15 contiguous nucleobases. In some embodiments, a portion is or comprises at least 16 contiguous nucleobases. In some embodiments, a portion is or comprises at least 17 contiguous nucleobases. In some embodiments, a portion is or comprises at least 18 contiguous nucleobases. In some embodiments, a portion is or comprises at least 19 contiguous nucleobases. In some embodiments, a portion is or comprises at least 20 contiguous nucleobases. In some embodiments, the base sequence of such a portion is characteristic of USH2A in that no other genomic or transcript sequences have the same sequence as the portion. In some embodiments, a portion of a gene that is complementary to an oligonucleotide is referred to as the target sequence of the oligonucleotide.

In some embodiments, an USH2A gene sequence (or a portion thereof, e.g., complementary to an USH2A oligonucleotide) is an USH2A gene sequence (or a portion thereof) known in the art or reported in the literature. Certain nucleotide and amino acid sequences of a human USH2A can be found in public sources, for example, one or more publicly available databases, e.g., GenBank, UniProt, OMEVI, etc. Those skilled in the art will appreciate that, for example, where a described nucleic acid sequence may be or include a genomic sequence, transcripts, splicing products, and/or encoded proteins, etc., may readily be appreciated from such genomic sequence.

In some embodiments, an USH2A gene, mRNA or protein or variant or isoform comprises a mutation.

The USH2A gene was initially described as comprising 21 exons, encoding a protein of 1546 amino acids. However, 51 additional exons at the 3′ end of USH2A were later discovered. Transcript of 72 exons, encoding a protein of 5202 amino acids, was reported. In addition, an alternative spliced exon 71 exists in mouse transcripts, expressed in the inner ear and well conserved in vertebrates. The long isoform b is characterized by containing a transmembrane region, followed by an intracellular domain with a PDZ-binding motif, which interacts with the PDZ domain of harmonin and whirlin, integrating USH2A into the USH protein network.

In some embodiments, mutations in the USH2A gene are the most frequent cause of Usher syndrome type IIA (2A), atypical Usher syndrome, and nonsyndromic retinitis pigmentosa. In some embodiments, the mutations are spread throughout the 72 USH2A exons and their flanking intronic sequences, and consist of nonsense and missense mutations, deletions, duplications, large rearrangements, and splicing variants. In some embodiments, Exon 13 is by far the most frequently mutated exon including two founder mutations, (c.2299delG (p.E767SfsX21) and c.2276G>T (p.C759F). The c.2299delG mutation found in exon 13 results in a frameshift causing a premature termination codon (e.g., a stop codon is gained) and is presumed to lead to nonsense mediated decay. Lenassi et al. (2014. The effect of the common c.2299delG mutation in USH2A on RNA splicing. Exp Eye Res 122:9-12) reported that in Usher patients the mutation leads to exon 12+exon 13 double-skipping during splicing, whereas in some patients a combination was found between exon 13 only-skip, and exon12/exon 13 double-skipping. It is reportedly not uncommon for exonic sequence alterations to cause aberrant splicing. Bioinformatics tools have reportedly predicted the c.2299delG change to disrupt an exonic splicing enhancer and to create an exonic splicing silencer within exon 13. Sequence analysis has reportedly shown that skipping only aberrant exon 13, carrying the mutation, results in removal of the frameshift mutation but also results in an in-frame link between exon 12 and exon 14. Double-skipping of exon 12 and exon 13 reportedly results in an out of frame deletion when exon 1 1 is linked to exon 14. Hence, in some embodiemtns, whereas skipping exon 13 is desired (when carrying the c.2299delG mutation) it is preferred that exon 12 is retained.

In some embodiments, an USH2A mRNA or protein is a transcription or translation product of an alternatively spliced variant or isoform. In some embodiments, an USH2A splicing variant is generated by an alternative splicing event not normally performed by a wild-type cell on a wild-type USH2A gene. In some embodiments, an USH2A transcript variant or isoform comprises one or more fewer or extra or different exons compared to a wild-type USH2A transcript. In some embodiments, an USH2A transcript variant or isoform comprises a frameshift mutation, leading to a premature stop codon. In some embodiments, a mutant USH2A transcript comprises a frameshift mutation, leading to a premature stop codon. In some embodiments, a mutant USH2A transcript comprises one or more mutations in exon 13.

In some embodiments, a mutant, variant or isoform of USH2A is incapable of performing at least one function, or has a decreased or increased ability to perform at least one function, compared to a wild-type USH2A. In some embodiments, a variant or isoform of USH2A is incapable of performing at least one function, or has a decreased ability to perform at least one function, compared to a wild-type USH2A. In some embodiments, a first mutant, isoform or variant of USH2A can be translated from a gene or transcript which comprises a deleterious mutation in an exon (e.g., exon 13) which decreases the ability of the protein to perform at least one function of a wild-type USH2A; and skipping of the deleterious exon (e.g., the exon comprising the deleterious mutation) in the transcript, and then translating from the transcript in which the deleterious exon is skipped produces a second USH2A variant (e.g., an internally truncated variant) in which the ability of the protein to perform at least one function of wild-type USH2A is at least partially restored, such that the second variant at least partially performs at least one function of a wild-type USH2A protein.

USH2A protein reportedly has partial sequence homology to both laminin epidermal growth factor and fibronectin motifs. In some embodiments, an USH2A protein performs at least one function akin to that of a laminin epidermal growth factor or fibronectin.

In some embodiments, provided technologies can modulate one or more of USH2A functions, e.g., through modulating sequence, expression, level and/or activity of an USH2A gene transcript or a product thereof. In some embodiments, an USH2A oligonucleotide is capable of increasing the level of skipping of a deleterious exon in an USH2A gene transcript or a gene product thereof, wherein the exon skipping product transcript and/or its encoded product thereof can provide a higher level of an USH2A function.

In some embodiments, an USH2A protein function includes but is not limited to: development and/or maintenance of supportive tissue in the inner ear and retina, a role in the basement membrane of the cochlea or retina or other tissue, interacting with collagen, usherin activity, interacting with the PDZ domain of harmonin and whirlin, integrating USH2A into the USH protein network, at least one function akin to that of a laminin epidermal growth factor or fibronectin, cell adhesion activity, and various roles in protein homodimerization activity, collagen binding, myosin binding, protein binding Cellular component, cytoplasm, stereocilium bundle, integral component of membrane, ciliary basal body, cell projection, stereocilium membrane, membrane, photoreceptor inner segment, stereocilia ankle link complex, plasma membrane, photoreceptor connecting cilium, stereocilia ankle link, extracellular region, basement membrane, USH2 complex, apical plasma membrane, periciliary membrane compartment, neuronal cell body, terminal bouton, response to stimulus, establishment of protein localization, hair cell differentiation, sensory perception of light stimulus, sensory perception of sound, inner ear receptor cell differentiation, photoreceptor cell maintenance, maintenance of animal organ identity, and visual perception, and any other function of USH2A described herein or known in the art. Without wishing to be bound by any particular theory, the present disclosure notes that wild-type USH2A may have at least one function which is not yet reported in the scientific literature.

In some embodiments, the retina is a thin neural tissue in the back of the eye comprising multiple layers of cells with distinct functions. It is reported that photoreceptor cells (e.g., rods and cones) within the retina are light-sensing neurons that are critical for visual phototransduction. Usherin is reported to be a cellular matrix protein expressed in photoreceptors that in some instances is essential for their long-term maintenance. In some embodiments, a USH2A oligonucleotide is useful for treatment of a pathology of the retina, including but not limited to pathologies of the retina described herein.

USH2A is reported to be expressed in tissues and organs such as: eye, retina, outer nuclear layer of the retina, ear, and cochlea. In some embodiments, the present disclosure pertains to the use of an USH2A oligonucleotide in increasing the level of skipping of a deleterious exon in an USH2A gene transcript or a gene product thereof, in eye, retina, outer nuclear layer of the retina, supportive tissue of the eye, supportive tissue of the ear, or cochlea. In some embodiments, the present disclosure pertains to the use of an USH2A oligonucleotide in increasing the level of skipping of a deleterious exon in an USH2A gene transcript or a gene product thereof, in a tissue and/or organ in a human patient in need thereof (e.g., a human patient suffering from or susceptible to an USH2A-related disease, disorder or condition), wherein the tissue and/or organ is any of: eye, retina, outer nuclear layer of the retina, supportive tissue of the eye, supportive tissue of the ear, or cochlea. In some embodiments, the present disclosure pertains to a method of treatment or amelioration of an USH2A-related disease, disorder or condition, comprising the step of increasing the level of skipping of a deleterious exon in an USH2A gene transcript or a gene product thereof, in a tissue and/or organ in a human patient in need thereof), wherein the tissue and/or organ is any of: eye, retina, outer nuclear layer of the retina, supportive tissue of the eye, supportive tissue of the ear, or cochlea. In various embodiments described herein, an USH2A gene transcript or gene product thereof is a mutant or comprises a mutation, including but not limited to mutation in exon 13 (Ex. 13).

In some embodiments, over 600 different mutations have been reported in USH2A, which are distributed throughout the gene, and include nonsense and missense mutations, deletions, duplications, large rearrangements, and variants that affect splicing.

In some embodiments, various deleterious (e.g., pathogenic) mutations have been reportedly identified in exon 13 of USH2A.

Mutations in USH2A exon 13 include but are not limited to: the 2299delG (predicted effect: p.E767SfsX21) and other mutations described herein or known in the art. Additional mutations reported for exon 13 include the missense mutations c.2276G>T (Amino acid change: p.C759F), and c.2522C >A (p.S841Y); nonsense mutation c.2242C>T (p.G1n748X); and mutations c.2541C>A (C847X); 2761del C (Leu921fs); c.2776C>T (p.R926C); and c.2802T>G (p.C934W).

In some embodiments, in various patients, alleles of USH2A can be homozygous, heterozygous, compound heterozygous, etc. In some embodiments, various patients have reportedly been identified who are homozygous for the same mutation in USH2A in both alleles (e.g., homozygous for the 2299delG mutation); and other patients have been reportedly identified which who different mutations in their two USH2A alleles (e.g., a 2299delG/C759F compound heterozygote).

Two non-syndromic autosomal recessive retinitis pigmentosa (ARRP) patients were reported, who were compound heterozygotes with C759F and frameshift mutations, which reportedly indicates that the frameshifts do not cause Usher type II, but only nonsyndromic RP if they are inherited together with the missense change C759F. In Spanish patients additional compound heterozygotes with C759F and nonsense, splicing, or missense mutations are reportedly associated with identical phenotypic features, reinforcing the hypothesis that mutations in the USH2A gene can result in ARRP without hearing loss.

The profile of USH2A gene mutations may reportedly differ significantly between Japanese patients and Caucasian populations.

USH2A is also reportedly expressed in at least these cells, tissues and organs: B lymphocytes; Dendritic cells; Endothelial cells; monocytes; B cells; myeloid cells; T cells; NK cells; early erythroid; T cells; 721 B lymphoblasts; Adipocyte; Adrenal Cortex; Adrenal gland; Amygdala; Appendix; Atrioventricular Node; BDCA4+ Dentritic Cells; Bone marrow; Bronchial Epithelial Cells; CD105+ Endothelial; CD14+ Monocytes; CD19+ B Cells (neg. sel.); CD33+ Myeloid; CD34+; CD4+ T cells; CD56+NK Cells; CD71+ Early Erythroid; CD8+ T cells; Cardiac Myocytes; Caudate nucleus; Cerebellum; Cerebellum Peduncles; Ciliary Ganglion; Cingulate Cortex; Colorectal adenocarcinoma; Dorsal Root Ganglion; Fetal Thyroid; Fetal brain; Fetal liver; Fetal lung; Globus Pallidus; Heart; Hypothalamus; Kidney; Leukemia chronic Myelogenous K-562; Leukemia promyelocytic-HL-60; Leukemia lymphoblastic (MOLT-4); Liver; Lung; Lymph node; Lymphoma Burkitt's (Daudi); Lymphoma Burkitt's (Raji); Medulla Oblongata; Occipital Lobe; Olfactory Bulb; Ovary; Pancreas; Pancreatic Islet; Parietal Lobe; Pituitary; Placenta; Pons; Prefrontal Cortex; Prostate; Salivary gland; Skeletal Muscle; Skin; Smooth Muscle; Spinal cord; Subthalamic Nucleus; Superior Cervical Ganglion; Temporal Lobe; Testis; Testis Germ Cell; Testis Interstitial; Testis Leydig Cell; Testis Seminiferous Tubule; Thalamus; Thymus; Thyroid; Tongue; Tonsil; Trachea; Trigeminal Ganglion; Uterus; Uterus Corpus; Whole Blood; Whole brain; Colon; Pineal; Pineal day; Blood; Brain; Pineal night; Retina; Small intestine; Leukemia chronic Myelogenous; Leukemia promyelocytic; and Leukemia lymphoblastic. In some embodiments, the present disclosure pertains to the use of an USH2A oligonucleotide in increasing the level of skipping of a deleterious exon in an USH2A gene transcript or a gene product thereof, in any of these tissues. In some embodiments, the present disclosure pertains to the use of an USH2A oligonucleotide in increasing the level of skipping of a deleterious exon in an USH2A gene transcript or a gene product thereof, in any of these tissues in a human patient in need thereof (e.g., a human patient suffering from or susceptible to an USH2A-related disease, disorder or condition). In some embodiments, the present disclosure pertains to a method of treatment or amelioration of an USH2A-related disease, disorder or condition, comprising the step of increasing the level of skipping of a deleterious exon in an USH2A gene transcript or a gene product thereof, in any of these tissues in a human patient in need thereof. In various embodiments described herein, an USH2A gene transcript or gene product thereof is a mutant or comprises a mutation, including but not limited to a P23H mutation.

In some embodiments, the present disclosure pertains to a method of administration of an USH2A oligonucleotide in a patient suffering from or susceptible to an USH2A-related disease, disorder, or condition, wherein the disease, disorder or condition manifests (e.g., is characterized by at least one symptom in) (A) the eye or the ear; and (B) another tissue in the body that expresses USH2A. In some embodiments, the present disclosure pertains to a method of administration of an USH2A oligonucleotide in a patient suffering from or susceptible to an USH2A-related disease, disorder, or condition, wherein the disease, disorder or condition manifests (e.g., is characterized by at least one symptom in) (A) the eye or the ear; and (B) another tissue in the body that expresses USH2A, wherein the USH2A oligonucleotide is administered to (A) the eye or the ear; and (B) the another tissue in the body that expresses USH2A. In some embodiments, the present disclosure pertains to a method of administration of an USH2A oligonucleotide in a patient suffering from or susceptible to an USH2A-related disease, disorder, or condition, wherein the disease, disorder or condition manifests (e.g., is characterized by at least one symptom in) (A) the eye or the ear; and (B) another tissue in the body that expresses USH2A, wherein the USH2A oligonucleotide is administered to (A) the eye or the ear; and (B) the another tissue in the body that expresses USH2A, wherein a first USH2A oligonucleotide administered to (A) the eye or the ear is in a formulation and/or delivered via a method and/or comprises an additional chemical moiety suitable for administration to the eye or the ear; and a second USH2A oligonucleotide administered to (B) the another tissue in the body that expresses USH2A is in a formulation and/or delivered via a method and/or comprises an additional chemical moiety suitable for administration to the another tissue in the body that expresses USH2A.

### USH2A-Related Conditions, Disorders or Diseases

In some embodiments, an USH2A-related disease, disorder or condition is any of various conditions, disorders or diseases are associated with a mutation(s) in USH2A; or, any disease, disorder or condition wherein at least one symptom is ameliorated by or the delayed in onset by increasing the level of skipping of a deleterious exon in an USH2A gene transcript or a gene product thereof, such a disease, disorder or condition includes retinopathy.

Various conditions, disorders or diseases are associated with USH2A, including but not limited to: Usher syndrome, Usher Syndrome Type IIA (2A), atypical Usher Syndrome, retinitis pigmentosa, and nonsyndromic retinitis pigmentosa (NSRP). In some embodiments, retinitis pigmentosa is an inherited retinal dystrophy (IRD); in some embodiments, an USH2A-related disease, disorder or condition is an inherited retinal dystrophy. In some embodiments, RP encompasses a group of progressive IRDs reportedly characterized by the primary degeneration of rod photoreceptors, followed by the loss of cone photoreceptors. The initial symptom is reportedly reduced night vision, which is followed by a progressive loss of the visual field in a concentric pattern.

Usher syndrome, also known as USH Syndrome, Hallgren syndrome, Usher-Hallgren syndrome, retinitis pigmentosa-dysacusis syndrome or dystrophia retinae dysacusis syndrome, is reportedly a genetic disorder caused by a mutation in any one of at least 11 genes resulting in a combination of hearing loss and visual impairment. It is the majority cause of deaf-blindness.

Usher syndrome is reportedly classed into three subtypes (I, II and III) according to the genes responsible and the onset of deafness. All three subtypes are reportedly caused by mutations in genes involved in the function of the inner ear and/or retina. These mutations are reportedly inherited in an autosomal recessive pattern.

Usher syndrome is reportedly named after Scottish ophthalmologist Charles Usher, who examined the pathology and transmission of the syndrome in 1914.

People with Usher I are reportedly born profoundly deaf and begin to lose their vision in the first decade of life. They also exhibit balance difficulties and learn to walk slowly as children, due to problems in their vestibular system. Usher syndrome type I reportedly can be caused by mutations in any one of several different genes: CDH23, MYO7A, PCDH15, USH1C and USH1G. These genes function in the development and maintenance of inner ear structures such as hair cells (stereocilia), which transmit sound and motion signals to the brain. Alterations in these genes can reportedly cause an inability to maintain balance (vestibular dysfunction) and hearing loss. The genes also reportedly play a role in the development and stability of the retina by influencing the structure and function of both the rod photoreceptor cells and supporting cells called the retinal pigmented epithelium. Mutations that affect the normal function of these genes can reportedly result in retinitis pigmentosa and resultant vision loss.

People with Usher Syndrome Type II (also referenced as Usher Syndrome II or Usher Syndrome 2) are reportedly not born deaf and are generally hard-of-hearing rather than deaf, and their hearing does not degrade over time; moreover, they do not seem to have noticeable problems with balance. They also reportedly begin to lose their vision later (in the second decade of life) and may preserve some vision even into middle age.

Usher syndrome type II may reportedly be caused by mutations in any of three different genes: USH2A, GPR98 and DFNB31. The protein reportedly encoded by the USH2A gene, usherin, is located in the supportive tissue in the inner ear and retina. Usherin is reportedly critical for the proper development and maintenance of these structures, which may help explain its role in hearing and vision loss.

Usher syndrome type II reportedly occurs at least as frequently as type I, but because type II may be underdiagnosed or more difficult to detect, it could be up to three times as common as type I.

In some embodiments, Usher syndrome type 2A is reportedly an autosomal recessive disease characterized by hearing loss at birth and progressive vision loss beginning in adolescence or adulthood. It is reportedly commonly caused by a mutation (2299del G) that introduces a stop codon in exon 13 and prevents translation of usherin protein, leading to progressive degeneration of photoreceptors

People with Usher syndrome III are reportedly not born deaf but experience a “progressive” loss of hearing, and roughly half have balance difficulties.

Mutations in only one gene, CLRN1, have reportedly been linked to Usher syndrome type III. CLRN1 reportedly encodes clarin-1, a protein important for the development and maintenance of the inner ear and retina.

Usher syndrome is reportedly characterized by hearing loss and a gradual visual impairment. The hearing loss is reportedly caused by a defective inner ear, whereas the vision loss results from retinitis pigmentosa (RP), a degeneration of the retinal cells. Usually, the rod cells of the retina are reportedly affected first, leading to early night blindness (nyctalopia) and the gradual loss of peripheral vision. In other cases, early degeneration of the cone cells in the macula reportedly occurs, leading to a loss of central acuity. In some cases, the foveal vision is spared, leading to “doughnut vision”; central and peripheral vision are intact, but an annulus exists around the central region in which vision is impaired.

Usher syndrome is inherited in an autosomal recessive pattern. Several genes have reportedly been associated with Usher syndrome using linkage analysis of patient families and DNA sequencing of the identified loci. A mutation in any one of these genes is reportedly likely to result in Usher syndrome.

The clinical subtypes Usher I and II are reportedly associated with mutations in any one of six (USH1B-G) and three (USH2A, C-D) genes, respectively, whereas only one gene, USH3A, has been linked to Usher III so far.

Using interaction analysis techniques, the identified gene products could reportedly be shown to interact with one another in one or more larger protein complexes. If one of the components is missing, this protein complex cannot fulfil its function in the living cell, and it probably comes to the degeneration the same. The function of this protein complex has reportedly been suggested to participate in the signal transduction or in the cell adhesion of sensory cells.

A study shows that three proteins reportedly related to Usher syndrome genes (PCDH15, CDH23, GPR98) are also involved in auditory cortex development, in mouse and macaque. Their lack of expression reportedly induces a decrease in the number of parvalbumin interneurons. Patients with mutations for these genes could have consequently auditory cortex defects.

The progressive blindness of Usher syndrome reportedly results from retinitis pigmentosa. The photoreceptor cells reportedly usually start to degenerate from the outer periphery to the center of the retina, including the macula. The degeneration is reportedly usually first noticed as night blindness (nyctalopia); peripheral vision is gradually lost, restricting the visual field (tunnel vision), which generally progresses to complete blindness. The qualifier pigmentosa reportedly reflects the fact that clumps of pigment may be visible by an ophthalmoscope in advanced stages of degeneration.

The hearing impairment reportedly associated with Usher syndrome is caused by damaged hair cells in the cochlea of the inner ear inhibiting electrical impulses from reaching the brain.

In some embodiments, it is reportedly helpful to diagnose children well before they develop the characteristic night blindness. Some preliminary studies have reportedly suggested as many as 10% of congenitally deaf children may have Usher syndrome. However, a misdiagnosis can reportedly have bad consequences.

One approach to diagnosing Usher syndrome is reportedly to test for the characteristic chromosomal mutations. An alternative approach is reportedly electroretinography, although this is often disfavored for children, since its discomfort can also make the results unreliable. Parental consanguinity is reportedly a significant factor in diagnosis. Usher syndrome I may reportedly be indicated if the child is profoundly deaf from birth and especially slow in walking.

Thirteen other syndromes may reportedly exhibit signs similar to Usher syndrome, including Alport syndrome, Alström syndrome, Bardet-Biedl syndrome, Cockayne syndrome, spondyloepiphyseal dysplasia congenita, Flynn-Aird syndrome, Friedreich ataxia, Hurler syndrome (MPS-1), Kearns-Sayre syndrome (CPEO), Norrie syndrome, osteopetrosis (Albers-Schonberg disease), Refsum disease (phytanic acid storage disease) and Zellweger syndrome (cerebrohepatorenal syndrome).

Usher syndrome (USH) is reportedly a combination of a progressive pigmentary retinopathy, indistinguishable from retinitis pigmentosa, and some degree of sensorineural hearing loss. USH can reportedly be subdivided in Usher type I (USHI), type II (USHII) and type III (USHIII), all of which are inherited as autosomal recessive traits. The three subtypes are reportedly genetically heterogeneous, with six loci so far identified for USHI, three for USHII and only one for USHIII. Mutations in a novel gene, USH2A, encoding the protein usherin, has been shown to be associated with USHII.

Usher syndrome type IIA (MIM: 276901) is an autosomal recessive disorder characterized by moderate to severe congenital deafness and progressive retinitis pigmentosa. Usher syndrome is also reportedly a degenerative disease of the retina. Mutations in the USH2A gene reportedly account for about half of the cases of Usher syndrome. Mutations in multiple exons including, 13, and 50 and introns including intron 40 are reportedly the leading cause of Usher syndrome. The present disclosure described, inter alia, stereopure USH2A oligonucleotides that skip exon 13.

Among other things, provided technologies are useful for treating or preventing a condition, disorder or disease associated with USH2A, e.g. Usher Syndrome. The protein encoded by the USH2A gene contains disease-associated mutations.

In some embodiments, an USH2A-related disorder is: Usher Syndrome

Symptoms of Usher Syndrome reportedly include: deafness, congenital deafness, retinitis pigmentosa, progressive retinitis pigmentosa, and a degenerative disease of the retina.

In some embodiments, an USH2A oligonucleotide, when administered to a patient suffering from or susceptible to Usher Syndrome, is capable of reducing at least one symptom of Usher Syndrome and/or capable of delaying or preventing the onset, worsening, and/or reducing the rate and/or degree of worsening of at least one symptom of Usher Syndrome.

In some embodiments, administration of an USH2A oligonucleotide improves, preserves, or prevents worsening of visual function; visual field; photoreceptor cell function; electroretinogram (ERG) response such as full field ERG measuring retina wide function, dark adapted ERG measuring scotopic rod function, or light adapted ERG measuring photopic cone function; visual acuity; and/or vision-related quality of life. In some embodiments, administration of an USH2A oligonucleotide inhibits, prevents, or delays progression of photoreceptor cell loss and/or deterioration of the retina outer nuclear layer (ONL).

In some embodiments, a symptom of an USH2A-related disease, disorder or condition [e.g., Usher Syndrome Type IIA (2A), atypical Usher syndrome, or nonsyndromic retinitis pigmentosa] is any symptom described herein, including but not limited to: blindness, night blindness (nyctalopia), photopsia, loss of peripheral vision, progressive visual loss, retinitis pigmentosa, vestibular dysfunction, sensorineural hearing loss, abnormal vestibular function, onset of night blindness, onset of visual field loss, decline in or loss of visual field, decline in or loss of visual acuity, abnormal eye fundus, increase in death of photoreceptors, loss of touch sensitivity and acuity, loss of tactile acuity, loss of vibration detection, compromised vibration detection threshold, low heat pain threshold, abnormal ankle links formation and cochlear development, abnormal periciliary maintenance, loss of mid-peripheral visual field, anatomical abnormalities in the central retina, visual hallucinations, animated visual hallucinations, Charles Bonnet syndrome, photophobia, and chromatopsia, hearing loss, retinal degeneration, and congenital hearing impairment.

In some embodiments, the symptoms of a patient suffering from or susceptible to an USH2A-related disease, disorder or condition can be evaluated using any method known in the art, including but not limited to: functional acuity score (FAS); functional field score (FFS); and functional vision score (FVS); Snellen visual acuity; Goldmann visual field area (V4c white test light), and 30-Hz (cone) full-field electroretinogram amplitude, electroretinogram (ERG), analysis of tissue samples, and light and/or immunofluorescence microscopy, immunohistochemistry and confocal microscopy, and terminal deoxynucleotidyl transferase-mediated dUTP nick-end labeling (TUNEL) assay, and optical coherence tomography (OCT).

In some embodiments, the present disclosure pertains to a method of administering a therapeutic amount of an USH2A oligonucleotide to a patient suffering from or susceptible to Usher Syndrome.

In some embodiments, a patient lacks a wild-type USH2A allele and has a mutant USH2A allele.

In some embodiments, a patient is homozygous, wherein both USH2A alleles are mutant.

The most common mutations in USH2A associated with Usher Syndrome Type IIA reportedly occur in exon 13 of the USH2A gene transcript. Mutations in USH2A exon 13 are reportedly present in both non-syndromic and syndromic forms of RP. Exon 13 mutations are reportedly some of the most common USH2A mutations. Mutations in exon 13 of the USH2A gene reportedly result in the absence of the usherin protein in the retinal photoreceptors and degeneration of the outer segment of photoreceptor cells.

Certain information related to USH2A and USH2A-related diseases, disorders or conditions has been reported in, for example: Adato A, Weston M D, Berry A, et al. (2000). “Three novel mutations and twelve polymorphisms identified in the USH2A gene in Israeli USH2 families”. Hum. Mutat. 15 (4): 388; Ahmed Z M, Riazuddin S, Riazuddin S, Wilcox E R (2004). “The molecular genetics of Usher syndrome”. Clin. Genet. 63 (6): 431-44; Aller E, Nijera C, Millin J M, et al. (2004). “Genetic analysis of 2299delG and C759F mutations (USH2A) in patients with visual and/or auditory impairments”. Eur. J. Hum. Genet. 12 (5): 407-10; Bernal S, Ayuso C, Antinolo G, et al. (2003). “Mutations in USH2A in Spanish patients with autosomal recessive retinitis pigmentosa: high prevalence and phenotypic variation”. J. Med. Genet. 40 (1): 8e-8; Bhattacharya G, Kalluri R, Orten D J, et al. (2004). “A domain-specific usherin/collagen IV interaction may be required for stable integration into the basement membrane superstructure”. J. Cell Sci. 117 (Pt 2): 233-42; and Bhattacharya G, Miller C, Kimberling W J, et al. (2002). “Localization and expression of usherin: a novel basement membrane protein defective in people with Usher's syndrome type IIa”. Hear. Res. 163 (1-2): 1-11; Dreyer B, Tranebjaerg L, Brox V, et al. (2001). “A common ancestral origin of the frequent and widespread 2299delG USH2A mutation”. Am. J. Hum. Genet. 69 (1): 228-34; Dreyer B, Tranebjaerg L, Rosenberg T, et al. (2000). “Identification of novel USH2A mutations: implications for the structure of USH2A protein”. Eur. J. Hum. Genet. 8 (7): 500-6; Eudy J D, Weston M D, Yao S, Hoover D M, Rehm H L, Ma-Edmonds M, Yan D, Ahmad I, Cheng J J, Ayuso C, Cremers C, Davenport S, Moller C, Talmadge C B, Beisel K W, Tamayo M, Morton C C, Swaroop A, Kimberling W J, Sumegi J (July 1998). “Mutation of a gene encoding a protein with extracellular matrix motifs in Usher syndrome type IIa”. Science. 280 (5370): 1753-7; Further reading; GRCh38: Ensembl release 89: ENSG00000042781—Ensembl, May 2017; GRCm38: Ensembl release 89: ENSMUSG00000026609—Ensembl, May 2017; Huang D, Eudy J D, Uzvolgyi E, et al. (2003). “Identification of the mouse and rat orthologs of the gene mutated in Usher syndrome type IIA and the cellular source of USH2A mRNA in retina, a target tissue of the disease”. Genomics. 80 (2): 195-203; Leroy B P, Aragon-Martin J A, Weston M D, et al. (2001). “Spectrum of mutations in USH2A in British patients with Usher syndrome type II”. Exp. Eye Res. 72 (5): 503-9; Liu X, Bulgakov O V, Darrow K N, Pawlyk B, Adamian M, Liberman M C, Li T (2007). “Usherin is required for maintenance of retinal photoreceptors and normal development of cochlear hair cells”. Proc Natl Acad Sci USA. 104 (11): 4413-8; Liu X Z, Hope C, Liang C Y, et al. (2000). “A mutation (2314delG) in the Usher syndrome type IIA gene: high prevalence and phenotypic variation”. Am. J. Hum. Genet. 64 (4): 1221-5; Michalski N, Michel V, Bahloul A, Lefèvre G, Barral J, Yagi H, Chardenoux S, Weil D, Martin P, Hardelin J P, Sato M, Petit C (2007). “Molecular characterization of the ankle-link complex in cochlear hair cells and its role in the hair bundle functioning”. J. Neurosci. 27 (24): 6478-88; Nijera C, Beneyto M, Blanca J, et al. (2002). “Mutations in myosin VIIA (MYO7A) and usherin (USH2A) in Spanish patients with Usher syndrome types I and II, respectively”. Hum. Mutat. 20 (1): 76-7; Pearsall N, Bhattacharya G, Wisecarver J, et al. (2003). “Usherin expression is highly conserved in mouse and human tissues”. Hear. Res. 174 (1-2): 55-63; Rivolta C, Berson E L, Dryja T P (2002). “Paternal uniparental heterodisomy with partial isodisomy of chromosome 1 in a patient with retinitis pigmentosa without hearing loss and a missense mutation in the Usher syndrome type II gene USH2A”. Arch. Ophthalmol. 120 (11): 1566-71; Rivolta C, Sweklo E A, Berson E L, Dryja T P (2001). “Missense mutation in the USH2A gene: association with recessive retinitis pigmentosa without hearing loss”. Am. J. Hum. Genet. 66 (6): 1975-8; Roland F P (1978). “Management of atypical pneumonias in view of the new entity “Legionnaire's disease””. Rhode Island Medical Journal. 61 (7): 270-2; van Wijk E, Pennings R J, te Brinke H, et al. (2004). “Identification of 51 novel exons of the Usher syndrome type 2A (USH2A) gene that encode multiple conserved functional domains and that are mutated in patients with Usher syndrome type II”. Am. J. Hum. Genet. 74 (4): 738-44; Weston M D, Eudy J D, Fujita S, Yao S, Usami S, Cremers C, Greenberg J, Ramesar R, Martini A, Moller C, Smith R J, Sumegi J, Kimberling W J (May 2000). “Genomic structure and identification of novel mutations in usherin, the gene responsible for Usher syndrome type IIa”. Am J Hum Genet. 66 (4): 1199-210; Liu et al. 1999 Am. J. Hum. Genet. 64: 1221-1225; Rivolta et al. 2000 Am. J. Human. Genet. 66: 1975-8; Eudy et al. 1998 Science 280: 1753-7; Liu et al. Am. J. Hum. Genet. 1999 64: 1221-5; Adato et al. 2000 Hum. Mutat. 15: 388-93; Dreyer et al. 2000 Eur. J. Hum. Genet. 8: 500-6; Leroy et al. 2001 Exp. Eye Res. 72: 503-9; ALLER, et al., Eur. J. Hum. Genet., 12:407-410 (2004); BERNAL, et al., J. Med. Genet., 40:e8 (2003); DAD, et al., Eur. J. Hum. Genet., 23:1646-1651 (2015); FUSTER-GARCIA, et al., Mol. Ther. Nuc. Acids, 8:529 (2017); GARCIA-GARCIA, et al., Orphanet J. Rare Dis., 6:65 (2011); MATHUR, et al., Biochim. Biophys. Acta., 1852:406-420 (2015); MILAN, et al., J. Ophthal., Article 417217 (2011); NAJERA, et al., Hum. Mut., Mut. In Brief 513 (2002); NAKANISHI, et al., J. Human Genet., 56:484-490 (2011); PENNINGS, et al., Hum. Mut., Mut. In Brief 730 (2004); PENNINGS, et al., Acta Ophthal. Scand., 82:131-139 (2004); SANDBERG, et al., Invest. Ophthal., V.3. Sci. 49:5532 (2008); SLIJKERMAN, et al., Mol. Ther. Nuc. Acids 5, e381 (2018); VAN WIJK, et al., Am. J. Hum. Genet., 74:738-744 (2004); VERKABEL, et al., Prog. Ret. Eye Res., 66:157-186 (2018); WESTON, et al., Am. J. Hum. Genet., 66:1199-1210 (2000); X U, et al., Mol. Vis., 17:1537-1552 (2011); YAN, et al., J. Hum. Genet., 54:732-738 (2009); and ZHAO, et al., J. Hum. Genet., 59:521-528 (2014). In some embodiments, an additional therapeutic agent or method includes but is not limited to any treatment described in any of these documents; and a tool, technique, method, cell or animal model useful for the evaluation of an oligonucleotide can include but is not limited to a tool, technique, method, cell or animal model described in any of these documents.

In some embodiments, an USH2A oligonucleotide capable of increasing the level of skipping of a deleterious exon in an USH2A gene is useful in a method of preventing or treating an USH2A-related condition, disorder or disease, e.g., Usher Syndrome.

In some embodiments, the present disclosure provides methods for preventing or treating an USH2A-related condition, disorder or disease, by administering to a subject suffering from or susceptible to such a condition, disorder or disease a therapeutically effective amount of a provided USH2A oligonucleotide or a composition thereof. In some embodiments, an oligonucleotide is a chirally controlled oligonucleotide. In some embodiments, an oligonucleotide is a chirally pure oligonucleotide. In some embodiments, a composition is a chirally controlled oligonucleotide composition. In some embodiments, a composition is a pharmaceutical composition. In some embodiments, in a composition oligonucleotides are independently in salt forms (e.g., sodium salts).

In some embodiments, the present disclosure pertains to a method of increasing the level of skipping of a deleterious exon in a mutant USH2A gene transcript or a gene product thereof in a body cell, tissue or organ affected by an USH2A-related disorder.

In some embodiments, a body cell, tissue or organ affected by an USH2A-related disorder does not exhibit normal function in an organism comprising a mutant USH2A gene.

In some embodiments, a body cell, tissue or organ affected by an USH2A-related disorder is the eye, retina, outer nuclear layer of the retina, supportive tissue of the eye, supportive tissue of the ear, or cochlea, or a portion or cell thereof.

In some embodiments, modulating the expression of an aberrant USH2A allele or transcript, for example, restores normal function of, for example, cells of the eye, retina, outer nuclear layer of the retina, supportive tissue of the eye, supportive tissue of the ear, or cochlea.

In some embodiments, the present disclosure encompasses a method of increasing the level of skipping of a deleterious exon in a mutant USH2A in a body cell, tissue or organ affected by an USH2A-related disorder.

In some embodiments, the present disclosure pertains to the use of an USH2A oligonucleotide in the treatment of any USH2A-related disorder, disease or condition, including but not limited to Usher Syndrome (e.g., Usher Syndrome Type 2A), atypical Usher syndrome, or nonsyndromic retinitis pigmentosa.

### Oligonucleotides

Among other things, the present disclosure provides oligonucleotides of various designs, which may comprises various nucleobases and patterns thereof, sugars and patterns thereof, internucleotidic linkages and patterns thereof, and/or additional chemical moieties and patterns thereof as described in the present disclosure. In some embodiments, provided USH2A oligonucleotides can mediate an increase in the level of skipping of a deleterious exon (e.g., human exon 13) in an USH2A gene and/or one or more of its products (e.g., an USH2A protein translated from an USH2A gene transcript in which a deleterious exon has been skipped). In some embodiments, provided USH2A oligonucleotides can mediate a decrease in the level of a nucleic acid (e.g., a transcript) that comprises a deleterious exon (e.g., human exon 13) in an USH2A gene and/or one or more of its products (e.g., an USH2A protein translated from an USH2A gene transcript in which a deleterious exon is included). In some embodiments, provided USH2A oligonucleotides can mediate an increase in the level of skipping of a deleterious exon in an USH2A gene and/or one or more of its products in any cell of a subject or patient. In some embodiments, a cell normally expresses USH2A or produces USH2A protein. In some embodiments, provided USH2A oligonucleotides can mediate an increase in the level of skipping of a deleterious exon in an USH2A gene transcript or a gene product thereof and has a base sequence which consists of, comprises, or comprises a portion (e.g., a span of 10, 11, 12, 13, 14, 15, 16, 17, 18, 19, 20 or more contiguous bases) of the base sequence of an USH2A oligonucleotide disclosed herein, wherein each T can be independently substituted with U and vice versa, and the oligonucleotide comprises at least one non-naturally-occurring modification of a base, sugar and/or internucleotidic linkage. In some embodiments, base sequences of USH2A oligonucleotides are at least 75%, 80%, 85%, 90%, or 95%, or 100% identical to or complementary to a USH2A sequence (e.g., a genetic sequence, a base sequence of a transcript, etc., or a portion thereof).

In some embodiments, an USH2A oligonucleotide is capable of mediating an increase in the level of skipping of a deleterious exon in a mutant USH2A gene transcript or a gene product thereof (e.g., a USHA protein translated from an USH2A gene transcript comprising a deleterious exon). In some embodiments, the deleterious exon in USH2A is exon 13.

In some embodiments, an USH2A oligonucleotide is selected from: WV-20891, WV-20892, WV-20902, WV-20908, WV-20988, WV-21008, WV-24297, WV-24368, WV-24376, WV-24366, WV-24375, WV-24360, WV-24298, WV-24381, WV-24382, WV-21100, WV-21105, WV-20885, and WV-30205.

In some embodiments, provided oligonucleotides, e.g., USH2A oligonucleotides, are antisense oligonucleotides (ASOs); they have a base sequence which is antisense to the target nucleic acid. In some embodiments, provided oligonucleotides, e.g., USH2A oligonucleotides, are double-stranded siRNAs. In some embodiments, provided oligonucleotides, e.g., USH2A oligonucleotides, are single-stranded siRNAs. Provided oligonucleotides and compositions thereof may be utilized for many purposes. For example, provided USH2A oligonucleotides can be co-administered or be used as part of a treatment regimen along with one or more treatment for Usher Syndrome or a symptom thereof, including but not limited to: aptamers, lncRNAs, lncRNA inhibitors, antibodies, peptides, small molecules, other oligonucleotides to USH2A or other targets, and/or other agents capable of inhibiting the expression of a mutant USH2A transcript, and/or increasing the level of expression of a mutant USH2A gene transcript in which a deleterious exon has been skipped, and/or reducing the level and/or activity of a mutant USH2A gene product, and/or inhibiting the expression of a gene or reducing the level of a gene product thereof which increases the expression, activity and/or level of a mutant USH2A gene transcript or a gene product thereof, or the level of another gene or gene product which is associated with an USH2A-related disorder.

In some embodiments, an USH2A oligonucleotide comprises a structural element or a portion thereof described herein, e.g., in a Table. In some embodiments, an USH2A oligonucleotide comprises a base sequence (or a portion thereof) described herein, wherein each T can be independently substituted with U and vice versa, a chemical modification or a pattern of chemical modifications (or a portion thereof), and/or a format or a portion thereof described herein. In some embodiments, an USH2A oligonucleotide has a base sequence which comprises the base sequence (or a portion thereof) wherein each T can be independently substituted with U, pattern of chemical modifications (or a portion thereof), and/or a format of an oligonucleotide disclosed herein, e.g., in a Table, or otherwise disclosed herein. In some embodiments, such oligonucleotides, e.g., USH2A oligonucleotides reduce expression, level and/or activity of a gene, e.g., an USH2A gene, or a gene product thereof.

Among other things, USH2A oligonucleotides may hybridize to their target nucleic acids (e.g., pre-mRNA, mature mRNA, etc.). For example, in some embodiments, an USH2A oligonucleotide can hybridize to an USH2A nucleic acid derived from a DNA strand (either strand of the USH2A gene). In some embodiments, an USH2A oligonucleotide can hybridize to an USH2A transcript. In some embodiments, an USH2A oligonucleotide can hybridize to an USH2A nucleic acid in any stage of RNA processing, including but not limited to a pre-mRNA or a mature mRNA. In some embodiments, an USH2A oligonucleotide can hybridize to any element of an USH2A nucleic acid or its complement, including but not limited to: a promoter region, an enhancer region, a transcriptional stop region, a translational start signal, a translation stop signal, a coding region, a non-coding region, an exon, an intron, an intron/exon or exon/intron junction, the 5′ UTR, or the 3′ UTR. In some embodiments, USH2A oligonucleotides can hybridize to their targets with no more than 2 mismatches. In some embodiments, USH2A oligonucleotides can hybridize to their targets with no more than one mismatch. In some embodiments, USH2A oligonucleotides can hybridize to their targets with no mismatches (e.g., when all C-G and/or A-T/U base paring).

In some embodiments, an oligonucleotide can hybridize to two or more variants of transcripts. In some embodiments, an USH2A oligonucleotide can hybridize to two or more or all variants of USH2A transcripts. In some embodiments, an USH2A oligonucleotide can hybridize to two or more or all variants of USH2A transcripts derived from the sense strand.

In some embodiments, an USH2A target of an USH2A oligonucleotide is an USH2A RNA which is not a mRNA.

In some embodiments, oligonucleotides, e.g., USH2A oligonucleotides, contain increased levels of one or more isotopes. In some embodiments, oligonucleotides, e.g., USH2A oligonucleotides, are labeled, e.g., by one or more isotopes of one or more elements, e.g., hydrogen, carbon, nitrogen, etc. In some embodiments, oligonucleotides, e.g., USH2A oligonucleotides, in provided compositions, e.g., oligonucleotides of a plurality of a composition, comprise base modifications, sugar modifications, and/or internucleotidic linkage modifications, wherein the oligonucleotides contain an enriched level of deuterium. In some embodiments, oligonucleotides, e.g., USH2A oligonucleotides, are labeled with deuterium (replacing —¹H with —²H) at one or more positions. In some embodiments, one or more ¹H of an oligonucleotide chain or any moiety conjugated to the oligonucleotide chain (e.g., a targeting moiety, etc.) is substituted with ²H. Such oligonucleotides can be used in compositions and methods described herein.

In some embodiments, the present disclosure provides an oligonucleotide composition comprising a plurality of oligonucleotides which:
1) have a common base sequence complementary to a target sequence (e.g., an USH2A target sequence) in a transcript; and
2) comprise one or more modified sugar moieties and/or modified internucleotidic linkages, wherein the oligonucleotide is capable of mediating skipping of a deleterious exon of an USH2A gene transcript.

In some embodiments, USH2A oligonucleotides having a common base sequence may have the same pattern of nucleoside modifications, e.g., sugar modifications, base modifications, etc. In some embodiments, a pattern of nucleoside modifications may be represented by a combination of locations and modifications. In some embodiments, a pattern of backbone linkages comprises locations and types (e.g., phosphate, phosphorothioate, substituted phosphorothioate, etc.) of each internucleotidic linkage.

In some embodiments, oligonucleotides of a plurality, e.g., in provided compositions, are of the same oligonucleotide type. In some embodiments, oligonucleotides of an oligonucleotide type have a common pattern of sugar modifications. In some embodiments, oligonucleotides of an oligonucleotide type have a common pattern of base modifications. In some embodiments, oligonucleotides of an oligonucleotide type have a common pattern of nucleoside modifications. In some embodiments, oligonucleotides of an oligonucleotide type have the same constitution. In some embodiments, oligonucleotides of an oligonucleotide type are identical. In some embodiments, oligonucleotides of a plurality are identical. In some embodiments, oligonucleotides of a plurality share the same constitution.

In some embodiments, as exemplified herein, USH2A oligonucleotides are chiral controlled, comprising one or more chirally controlled internucleotidic linkages. In some embodiments, USH2A oligonucleotides are stereochemically pure. In some embodiments, USH2A oligonucleotides are substantially separated from other stereoisomers.

In some embodiments, USH2A oligonucleotides comprise one or more modified nucleobases, one or more modified sugars, and/or one or more modified internucleotidic linkages.

In some embodiments, USH2A oligonucleotides comprise one or more modified sugars. In some embodiments, oligonucleotides of the present disclosure comprise one or more modified nucleobases. Various modifications can be introduced to a sugar and/or nucleobase in accordance with the present disclosure. For example, in some embodiments, a modification is a modification described in U.S. Pat. No. 9,006,198. In some embodiments, a modification is a modification described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612, the sugar, base, and internucleotidic linkage modifications of each of which are independently incorporated herein by reference.

As used in the present disclosure, in some embodiments, “one or more” is 1-200, 1-150, 1-100, 1-90, 1-80, 1-70, 1-60, 1-50, 1-40, 1-30, or 1, 2, 3, 4, 5, 6, 7, 8, 9, 10, 11, 12, 13, 14, 15, 16, 17, 18, 19, 20, 21, 22, 23, 24, or 25. In some embodiments, “one or more” is one. In some embodiments, “one or more” is two. In some embodiments, “one or more” is three. In some embodiments, “one or more” is four. In some embodiments, “one or more” is five. In some embodiments, “one or more” is six. In some embodiments, “one or more” is seven. In some embodiments, “one or more” is eight. In some embodiments, “one or more” is nine. In some embodiments, “one or more” is ten. In some embodiments, “one or more” is at least one. In some embodiments, “one or more” is at least two. In some embodiments, “one or more” is at least three. In some embodiments, “one or more” is at least four. In some embodiments, “one or more” is at least five. In some embodiments, “one or more” is at least six. In some embodiments, “one or more” is at least seven. In some embodiments, “one or more” is at least eight. In some embodiments, “one or more” is at least nine. In some embodiments, “one or more” is at least ten.

As used in the present disclosure, in some embodiments, “at least one” is 1-200, 1-150, 1-100, 1-90, 1-80, 1-70, 1-60, 1-50, 1-40, 1-30, or 1, 2, 3, 4, 5, 6, 7, 8, 9, 10, 11, 12, 13, 14, 15, 16, 17, 18, 19, 20, 21, 22, 23, 24, or 25. In some embodiments, “at least one” is one. In some embodiments, “at least one” is two. In some embodiments, “at least one” is three. In some embodiments, “at least one” is four. In some embodiments, “at least one” is five. In some embodiments, “at least one” is six. In some embodiments, “at least one” is seven. In some embodiments, “at least one” is eight. In some embodiments, “at least one” is nine. In some embodiments, “at least one” is ten.

In some embodiments, a USH2A oligonucleotide or composition is or comprises a USH2A oligonucleotide or composition described in a Table.

As demonstrated in the present disclosure, in some embodiments, a provided oligonucleotide (e.g., an USH2A oligonucleotide) is characterized in that, when it is contacted with an USH2A transcript in a splicing system, skipping of a deleterious exon in an USH2A gene transcript (e.g., an USH2A gene transcript for an USH2A oligonucleotide, a mutant USH2A gene transcript comprising disease-associated mutations, etc.) is improved relative to that observed under reference conditions (e.g., selected from the group consisting of absence of the composition, presence of a reference composition, and combinations thereof). In some embodiments, skipping of a deleterious exon in an USH2A gene transcript is increased 10%, 20%, 30%, 40%, 50%, 60%, 70%, 80%, 90%, 100%, or 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 200, 300, 400, 500, 600, 700, 800, 900, 1000 fold or more compared to absence of the oligonucleotide, or presence of a reference oligonucleotide (e.g., WV-20781).

In some embodiments, provided oligonucleotides can provide high levels of exon skipping, and/or high selectivity for skipping of particular exons (e.g., in some embodiments, high selectivity for skipping exon 13 (low levels of skipping other exon(s), e.g., exon 12, exon 12 and exon 13, etc.)).

Without wishing to be bound by any particular theory, the present disclosure notes that a small degree of skipping of exons other than exon 13 may occur in eye cells. In the absence of any introduced oligonucleotide, a small amount of skipping of exon 12 may occur. In some embodiments, if a USH2A transcript comprises a deleterious mutation in exon 13, skipping of exon 12 is non-productive, as it does not correct the defect in exon 13 and introduces a frameshift error.

In some embodiments, an USH2A oligonucleotide capable of skipping exon 13 demonstrates only a small amount of skipping of exon 12 (which can be, in some embodiments, experimentally evaluated as a small amount of simultaneous skipping of exons 12 and 13). In some embodiments, ratio of exon 13 skipping over exon 12 skipping (and/or exon 12 and exon 13 skipping) is about 2-10 fold or more (e.g., at least about 2, 3, 4, 5, 6, 7, 8, 9, 10 fold or more).

In some embodiments, certain data of various USH2A oligonucleotides to skip exon 13 are described in various Tables (e.g., Tables 1 to 9, and 13 on). In some embodiments, certain data of various USH2A oligonucleotides to simultaneously skip exons 12 and 13 are described, e.g., in Tables 10 to 12 (including Table 12A and Table 12B). Table 12B, for example, shows that some USH2A oligonucleotides demonstrated a ratio of skipping only exon 13/simultaneous skipping of exons 12 and 13 of: 4.4 or 4.1 (for WV-20908 and WV-20902, respectively), compared to 2.1 for a reference USH2A oligonucleotide (WV-20781).

In some embodiments, several USH2A oligonucleotides disclosed herein (e.g., WV-20908, WV-20902, WV-20892, WV-20891, and WV-20885, etc.) demonstrated both higher overall skipping of USH2A exon 13 than the reference oligonucleotide (e.g., WV-20781), but also higher specificity of skipping (e.g., skipping only exon 13 compared to simultaneous skipping of exons 12 and 13) than the reference oligonucleotide (e.g., WV-20781).

In some embodiments, alternatively or additionally, skipping selectivity (e.g., skipping of exon 13 only over skipping of both exon 12 and exon 13) is increased 10%, 20%, 30%, 40%, 50%, 60%, 70%, 80%, 90%, 100%, or 2, 3, 4, 5, 6, 7, 8, 9, 10 fold or more compared to absence of the oligonucleotide, or presence of a reference oligonucleotide (e.g., WV-20781 (which, as appreciated by those skilled in the art, represents a stereorandom composition comprising various diastereomers randomly (not chirally controlled)).

In some embodiments, oligonucleotides are provided as salt forms. In some embodiments, oligonucleotides are provided as salts comprising negatively-charged internucleotidic linkages (e.g., phosphorothioate internucleotidic linkages, natural phosphate linkages, etc.) existing as their salt forms. In some embodiments, oligonucleotides are provided as pharmaceutically acceptable salts. In some embodiments, oligonucleotides are provided as metal salts. In some embodiments, oligonucleotides are provided as sodium salts. In some embodiments, oligonucleotides are provided as metal salts, e.g., sodium salts, wherein each negatively-charged internucleotidic linkage is independently in a salt form (e.g., for sodium salts, —O—P(O)(SNa)—O— for a phosphorothioate internucleotidic linkage, —O—P(O)(ONa)—O— for a natural phosphate linkage, etc.).

### Base Sequences

In some embodiments, an USH2A oligonucleotide comprises a base sequence described herein or a portion (e.g., a span of 5-50, 5-40, 5-30, 5-20, or 5, 6, 7, 8, 9, 10, 11, 12, 13, 14, 15, 16, 17, 18, 19, 20, or at least 10, at least 15, contiguous nucleobases) thereof with 0-5 (e.g., 0, 1, 2, 3, 4 or 5) mismatches, wherein each T can be independently substituted with U and vice versa. In some embodiments, an USH2A oligonucleotide comprises a base sequence described herein, or a portion thereof, wherein a portion is a span of at least 10 contiguous nucleobases, or a span of at least 15 contiguous nucleobases with 1-5 mismatches. In some embodiments, provided oligonucleotides comprise a base sequence described herein, or a portion thereof, wherein a portion is a span of at least 10 contiguous nucleobases, or a span of at least 10 contiguous nucleobases with 1-5 mismatches, wherein each T can be independently substituted with U and vice versa. In some embodiments, base sequences of oligonucleotides comprise or consists of 10-50 (e.g., about or at least 10, 11, 12, 13, 14, 15, 16, 17, 18, 19, 20, 21, 22, 23, 24, 25, 30, 35, 40, 45; in some embodiments, at least 15; in some embodiments, at least 16; in some embodiments, at least 17; in some embodiments, at least 18; in some embodiments, at least 19; in some embodiments, at least 20; in some embodiments, at least 21; in some embodiments, at least 22; in some embodiments, at least 23; in some embodiments, at least 24; in some embodiments, at least 25) contiguous bases of a base sequence that is identical to or complementary to a base sequence of an USH2A gene or a transcript (e.g., mRNA) thereof. In some embodiments, the base sequence of an oligonucleotide is or comprises a complementary sequence that is complementary to a target sequence in an USH2A gene or a transcript thereof. In some embodiments, the complementary sequence is 10, 11, 12, 13, 14, 15, 16, 17, 18, 19 or 20 or more nucleobases in length.

In certain embodiments, a base sequence of an USH2A oligonucleotide is at least about 50%, about 60%, about 70%, about 75%, about 80%, about 85%, about 90%, about 91%, about 92%, about 93%, about 94%, about 95%, about 96%, about 97%, about 98%, or about 99%, or 100% complementary or identical to a target nucleic acid sequence (e.g., a base sequence of an USH2A transcript)

Base sequences of provided oligonucleotides, as appreciated by those skilled in the art, typically have sufficient length and complementarity to their targets, e.g., RNA transcripts (e.g., pre-mRNA, mature mRNA, etc.) to mediate skipping of a deleterious exon in an USH2A gene transcript. In some embodiments, the base sequence of an USH2A oligonucleotide has a sufficient length and identity to an USH2A gene transcript target to mediate skipping of a deleterious exon in an USH2A gene transcript. In some embodiments, the USH2A oligonucleotide is complementary to a portion of an USH2A gene transcript (an USH2A gene transcript target sequence). In some embodiments, the base sequence of an USH2A oligonucleotide has 90% or more identity with the base sequence of an oligonucleotide disclosed in a Table, wherein each T can be independently substituted with U and vice versa. In some embodiments, the base sequence of an USH2A oligonucleotide has 95% or more identity with the base sequence of an oligonucleotide disclosed in a Table, wherein each T can be independently substituted with U and vice versa. In some embodiments, the base sequence of an USH2A oligonucleotide comprises a continuous span of 15 or more bases of an oligonucleotide disclosed in a Table, wherein each T can be independently substituted with U and vice versa, except that one or more bases within the span are abasic (e.g., a nucleobase is absent from a nucleotide). In some embodiments, the base sequence of an USH2A oligonucleotide comprises a continuous span of 19 or more bases of an USH2A oligonucleotide disclosed herein, except that one or more bases within the span are abasic (e.g., a nucleobase is absent from a nucleotide). In some embodiments, the base sequence of an USH2A oligonucleotide comprises a continuous span of 19 or more bases of an oligonucleotide disclosed herein, wherein each T can be independently substituted with U and vice versa, except for a difference in the 1 or 2 bases at the 5′ end and/or 3′ end of the base sequences.

In some embodiments, the base sequence of an oligonucleotide is, comprises, or comprises 10-20, e.g., 10, 11, 12, 13, 14, 15, 16, 17, 18, 19 or 20 contiguous bases of AAGCCCUAAAGAUAAAAUAU (SEQ ID NO: 1), wherein each U may be independently replaced with T and vice versa.

In some embodiments, the base sequence of an oligonucleotide is, comprises, or comprises 10-20, e.g., 10, 11, 12, 13, 14, 15, 16, 17, 18, 19 or 20 contiguous bases of AAUACAUUUCUUUCUUACCU (SEQ ID NO: 2), wherein each U may be independently replaced with T and vice versa.

In some embodiments, the base sequence of an oligonucleotide is, comprises, or comprises 10-20, e.g., 10, 11, 12, 13, 14, 15, 16, 17, 18, 19 or 20 contiguous bases of ACAUCCAACAUCAUUAAAGC (SEQ ID NO: 3), wherein each U may be independently replaced with T and vice versa.

In some embodiments, the base sequence of an oligonucleotide is, comprises, or comprises 10-20, e.g., 10, 11, 12, 13, 14, 15, 16, 17, 18, 19 or 20 contiguous bases of AGCUUCGGAGAAAUUUAAAUC (SEQ ID NO: 4), wherein each U may be independently replaced with T and vice versa.

In some embodiments, the base sequence of an oligonucleotide is, comprises, or comprises 10-20, e.g., 10, 11, 12, 13, 14, 15, 16, 17, 18, 19 or 20 contiguous bases of AGCUUCGGAGAAAUUUAAAUC (SEQ ID NO: 5), wherein each U may be independently replaced with T and vice versa.

In some embodiments, the base sequence of an oligonucleotide is, comprises, or comprises 10-20, e.g., 10, 11, 12, 13, 14, 15, 16, 17, 18, 19 or 20 contiguous bases of AGGAUUGCAGAAUUUGUUCA (SEQ ID NO: 6), wherein each U may be independently replaced with T and vice versa.

In some embodiments, the base sequence of an oligonucleotide is, comprises, or comprises 10-20, e.g., 10, 11, 12, 13, 14, 15, 16, 17, 18, 19 or 20 contiguous bases of AGGAUUGCAGAAUUUGUUCA (SEQ ID NO:7), wherein each U may be independently replaced with T and vice versa.

In some embodiments, the base sequence of an oligonucleotide is, comprises, or comprises 10-20, e.g., 10, 11, 12, 13, 14, 15, 16, 17, 18, 19 or 20 contiguous bases of AUCCAAAAUUGCAAUGAUCA (SEQ ID NO: 8), wherein each U may be independently replaced with T and vice versa.

In some embodiments, the base sequence of an oligonucleotide is, comprises, or comprises 10-20, e.g., 10, 11, 12, 13, 14, 15, 16, 17, 18, 19 or 20 contiguous bases of AUUUCUUUCUUACCUGGUUG (SEQ ID NO: 9), wherein each U may be independently replaced with T and vice versa.

In some embodiments, the base sequence of an oligonucleotide is, comprises, or comprises 10-20, e.g., 10, 11, 12, 13, 14, 15, 16, 17, 18, 19 or 20 contiguous bases of CAACAUCAUUAAAGCUUCGG (SEQ ID NO: 10), wherein each U may be independently replaced with T and vice versa.

In some embodiments, the base sequence of an oligonucleotide is, comprises, or comprises 10-20, e.g., 10, 11, 12, 13, 14, 15, 16, 17, 18, 19 or 20 contiguous bases of CACCUAAGCCCUAAAGAUAA (SEQ ID NO: 11), wherein each U may be independently replaced with T and vice versa.

In some embodiments, the base sequence of an oligonucleotide is, comprises, or comprises 10-20, e.g., 10, 11, 12, 13, 14, 15, 16, 17, 18, 19 or 20 contiguous bases of GAGGAUUGCAGAAUUUGUUC (SEQ ID NO: 12), wherein each U may be independently replaced with T and vice versa.

In some embodiments, the base sequence of an oligonucleotide is, comprises, or comprises 10-20, e.g., 10, 11, 12, 13, 14, 15, 16, 17, 18, 19 or 20 contiguous bases of GAUCACACCUAAGCCCUAAA (SEQ ID NO: 13), wherein each U may be independently replaced with T and vice versa.

In some embodiments, the base sequence of an oligonucleotide is, comprises, or comprises 10-20, e.g., 10, 11, 12, 13, 14, 15, 16, 17, 18, 19 or 20 contiguous bases of GAUUGCAGAAUUUGUUCACU (SEQ ID NO: 14), wherein each U may be independently replaced with T and vice versa.

In some embodiments, the base sequence of an oligonucleotide is, comprises, or comprises 10-20, e.g., 10, 11, 12, 13, 14, 15, 16, 17, 18, 19 or 20 contiguous bases of GCAAUGAUCACACCUAAGCC (SEQ ID NO: 15), wherein each U may be independently replaced with T and vice versa.

In some embodiments, the base sequence of an oligonucleotide is, comprises, or comprises 10-20, e.g., 10, 11, 12, 13, 14, 15, 16, 17, 18, 19 or 20 contiguous bases of GCUUCGGAGAAAUUUAAAUC (SEQ ID NO: 16), wherein each U may be independently replaced with T and vice versa.

In some embodiments, the base sequence of an oligonucleotide is, comprises, or comprises 10-20, e.g., 10, 11, 12, 13, 14, 15, 16, 17, 18, 19 or 20 contiguous bases of GGAAUCACACUCACACAUCU (SEQ ID NO: 17), wherein each U may be independently replaced with T and vice versa.

In some embodiments, the base sequence of an oligonucleotide is, comprises, or comprises 10-20, e.g., 10, 11, 12, 13, 14, 15, 16, 17, 18, 19 or 20 contiguous bases of GGAUUGCAGAAUUUGUUCAC (SEQ ID NO: 18), wherein each U may be independently replaced with T and vice versa.

In some embodiments, the base sequence of an oligonucleotide is, comprises, or comprises 10-20, e.g., 10, 11, 12, 13, 14, 15, 16, 17, 18, 19 or 20 contiguous bases of GGAUUGCAGAAUUUGUUCA (SEQ ID NO: 19), wherein each U may be independently replaced with T and vice versa.

In some embodiments, the base sequence of an oligonucleotide is, comprises, or comprises 10-20, e.g., 10, 11, 12, 13, 14, 15, 16, 17, 18, 19 or 20 contiguous bases of UACCUGGUUGACACUGAUUA (SEQ ID NO: 20), wherein each U may be independently replaced with T and vice versa.

In some embodiments, the base sequence of an oligonucleotide is, comprises, or comprises 10-20, e.g., 10, 11, 12, 13, 14, 15, 16, 17, 18, 19 or 20 contiguous bases of UACCUGGUUGACACUGAUUA (SEQ ID NO: 21), wherein each U may be independently replaced with T and vice versa.

In some embodiments, the base sequence of an oligonucleotide is, comprises, or comprises 10-20, e.g., 10, 11, 12, 13, 14, 15, 16, 17, 18, 19 or 20 contiguous bases of UCUUUUUUGCACUCACACUG (SEQ ID NO: 22), wherein each U may be independently replaced with T and vice versa.

In some embodiments, the base sequence of an oligonucleotide is, comprises, or comprises 10-20, e.g., 10, 11, 12, 13, 14, 15, 16, 17, 18, 19 or 20 contiguous bases of UGAGGAUUGCAGAAUUUGUU (SEQ ID NO: 23), wherein each U may be independently replaced with T and vice versa.

In some embodiments, the base sequence of an oligonucleotide is, comprises, or comprises 10-20, e.g., 10, 11, 12, 13, 14, 15, 16, 17, 18, 19 or 20 contiguous bases of UGAGGAUUGCAGAAUUUGUU (SEQ ID NO: 24), wherein each U may be independently replaced with T and vice versa.

In some embodiments, the base sequence of an oligonucleotide is, comprises, or comprises 10-20, e.g., 10, 11, 12, 13, 14, 15, 16, 17, 18, 19 or 20 contiguous bases of UGCAGAAUUUGUUCACUGAG (SEQ ID NO: 25), wherein each U may be independently replaced with T and vice versa.

In some embodiments, the base sequence of an oligonucleotide is, comprises, or comprises 10-20, e.g., 10, 11, 12, 13, 14, 15, 16, 17, 18, 19 or 20 contiguous bases of UUGCAGAAUUUGUUCACUGA (SEQ ID NO: 26), wherein each U may be independently replaced with T and vice versa.

In some embodiments, the base sequence of an oligonucleotide is, comprises, or comprises 10-20, e.g., 10, 11, 12, 13, 14, 15, 16, 17, 18, 19 or 20 contiguous bases of UUUCUUACCUGGUUGACACU (SEQ ID NO: 27), wherein each U may be independently replaced with T and vice versa.

In some embodiments, the present disclosure pertains to an oligonucleotide having a base sequence which comprises the base sequence of any oligonucleotide disclosed herein, wherein each U may be independently replaced with T and vice versa.

In some embodiments, the present disclosure pertains to an oligonucleotide having a base sequence which is the base sequence of any oligonucleotide disclosed herein, wherein each U may be independently replaced with T and vice versa.

In some embodiments, the present disclosure pertains to an oligonucleotide having a base sequence which comprises at least 15 contiguous bases of the base sequence of any oligonucleotide disclosed herein, wherein each U may be independently replaced with T and vice versa.

In some embodiments, the present disclosure pertains to an oligonucleotide having a base sequence which is at least 90% identical to the base sequence of any oligonucleotide disclosed herein, wherein each U may be independently replaced with T and vice versa.

In some embodiments, the present disclosure pertains to an oligonucleotide having a base sequence which is at least 95% identical to the base sequence of any oligonucleotide disclosed herein, wherein each U may be independently replaced with T and vice versa.

In some embodiments, a base sequence of an oligonucleotide is, comprises, or comprises 10-20, e.g., 10, 11, 12, 13, 14, 15, 16, 17, 18, 19 or 20 contiguous bases of the base sequence of any oligonucleotide describer herein, wherein each U may be independently replaced with T and vice versa.

In some embodiments, an USH2A oligonucleotide is any USH2A oligonucleotide provided herein.

In some embodiments, an USH2A oligonucleotide is selected from: WV-20891, WV-20892, WV-20902, WV-20908, WV-20988, WV-21008, WV-24297, WV-24368, WV-24376, WV-24366, WV-24375, WV-24360, WV-24298, WV-24381, WV-24382, WV-21100, WV-21105, and WV-20885.

In some embodiments, the base sequence of an USH2A oligonucleotide is complementary to that of an USH2A gene transcript or a portion thereof.

In some embodiments, an USH2A oligonucleotide capable of mediating skipping of USH2A exon 13 has a sequence which hybridizes to (e.g., is complementary to a sequence of) an USH2A gene transcript sequence within exon 13, a sequence within an intron immediately adjacent to exon 13, or a sequence spanning the boundary between USH2A exon 13 and an intron immediately adjacent to exon 13.

In some embodiments, the boundaries between exon 13 and the introns immediately 5′ or 3′ to exon 13 are reported in Weston et al. Am. J. Hum. Genet. 66:1199-1210, 2000.

In some embodiments, an USH2A oligonucleotide capable of mediating skipping of USH2A exon 13 has a sequence which hybridizes to (e.g., is complementary to a sequence of) an USH2A gene transcript sequence within an intron immediately adjacent to exon 13. Non-limiting examples of such an oligonucleotide include but are not limited to: WV-20781, and other oligonucleotides having the same base sequence, or having a base sequence complementary to an USH2A gene transcript sequence within an intron immediately adjacent to exon 13.

In some embodiments, an USH2A oligonucleotide capable of mediating skipping of USH2A exon 13 has a sequence which hybridizes to (e.g., is complementary to a sequence of) an USH2A gene transcript sequence spanning the boundary between USH2A exon 13 and the intron immediately 5′ to exon 13. Non-limiting examples of such an oligonucleotide include but are not limited to: WV-20880, WV-20881, WV-20882, WV-20883, and other oligonucleotides having the same base sequence, or having a base sequence complementary to an USH2A gene transcript sequence spanning the boundary between USH2A exon 13 and the intron immediately 5′ to exon 13.

In some embodiments, an USH2A oligonucleotide capable of mediating skipping of USH2A exon 13 has a sequence which hybridizes to (e.g., is complementary to a sequence of) an USH2A gene transcript sequence within exon 13. Non-limiting examples of such an oligonucleotide include but are not limited to: WV-20884, WV-20885, WV-20886, WV-20887, WV-20888, WV-20889, WV-20890, WV-20891, WV-20892, WV-20893, WV-20894, WV-20895, WV-20896, WV-20897, WV-20898, WV-20899, WV-20900, WV-20901, WV-20902, WV-20903, WV-20904, WV-20905, WV-20906, WV-20907, WV-20908, WV-20909, WV-20910, WV-20911, WV-20912, WV-20913, WV-20914, WV-20915, WV-20916, WV-20917, WV-20918, WV-20919, WV-20920, WV-20921, WV-20922, WV-20923, WV-20924, WV-20925, WV-20926, WV-20927, WV-20928, WV-20929, WV-20930, WV-20931, WV-20932, WV-20933, WV-20934, WV-20935, WV-20936, WV-20937, WV-20938, WV-20939, WV-20940, WV-20941, WV-20942, WV-20943, WV-20944, WV-20945, WV-20946, WV-20947, WV-20948, WV-20949, WV-20950, WV-20951, WV-20952, WV-20953, WV-20954, WV-20955, WV-20956, WV-20957, WV-20958, WV-20959, WV-20960, WV-20961, WV-20962, WV-20963, WV-20964, WV-20965, WV-20966, WV-20967, WV-20968, WV-20969, WV-20970, WV-20971, WV-20972, WV-20973, WV-20974, WV-20975, WV-20976, WV-20977, WV-20978, WV-20979, WV-20980, WV-20981, WV-20982, WV-20983, WV-20984, WV-20985, WV-20986, WV-20987, WV-20988, WV-20989, WV-20990, WV-20991, WV-20992, WV-20993, WV-20994, WV-20995, WV-20996, WV-20997, WV-20998, WV-20999, WV-21000, WV-21001, WV-21002, WV-21003, WV-21004, WV-21005, WV-21006, WV-21007, and other oligonucleotides having the same base sequence, or having a base sequence complementary to USH2A exon 13.

In some embodiments, an USH2A oligonucleotide capable of mediating skipping of USH2A exon 13 has a sequence which hybridizes to (e.g., is complementary to a sequence of) an USH2A gene transcript sequence spanning the boundary between USH2A exon 13 and an intron immediately adjacent to exon 13. Non-limiting examples of such an oligonucleotide include but are not limited to: WV-21009, WV-21010, and WV-21011, and other oligonucleotides having the same base sequence, or having a base sequence complementary to an USH2A gene transcript sequence spanning the boundary between USH2A exon 13 and an intron immediately adjacent to exon 13.

In some embodiments, an USH2A oligonucleotide sequence within an intron immediately adjacent to exon 13. Non-limiting examples of such an oligonucleotide include but are not limited to: WV-21012, and other oligonucleotides having the same base sequence, or having a base sequence complementary to an USH2A oligonucleotide sequence within an intron immediately adjacent to exon 13.

In some embodiments, an USH2A oligonucleotide comprises a base sequence or portion (e.g., a portion comprising 10, 11, 12, 13, 14, 15, 16, 17, 18, 19, or 20 nucleobases) thereof described in the Tables, wherein each U may be independently replaced with T and vice versa, and/or a sugar, nucleobase, and/or internucleotidic linkage modification and/or a pattern thereof described in the Tables, and/or an additional chemical moiety (in addition to an oligonucleotide chain, e.g., a target moiety, a lipid moiety, a carbohydrate moiety, etc.) described in the Tables.

In some embodiments, the terms “complementary,” “fully complementary” and “substantially complementary” may be used with respect to the base matching between an oligonucleotide (e.g., an USH2A oligonucleotide) and a target sequence (e.g., an USH2A target sequence), as will be understood by those skilled in the art from the context of their use. As a non-limiting example, if a target sequence has, for example, a base sequence of 5′-GUGCUAGUAGCCAACCCCC-3′ (SEQ ID NO: 28), an oligonucleotide with a base sequence of 5′-GGGGGTTGGCTACTAGCAC-3′ (SEQ ID NO: 29) is complementary (fully complementary) to such a target sequence. It is noted that substitution of T for U, or vice versa, generally does not alter the amount of complementarity. As used herein, an oligonucleotide that is “substantially complementary” to a target sequence is largely or mostly complementary but not 100% complementary. In some embodiments, a sequence (e.g., an USH2A oligonucleotide) which is substantially complementary has 1, 2, 3, 4 or 5 mismatches when aligned to its target sequence. In some embodiments, an USH2A oligonucleotide has a base sequence which is substantially complementary to an USH2A target sequence. In some embodiments, an USH2A oligonucleotide has a base sequence which is substantially complementary to the complement of the sequence of an USH2A oligonucleotide disclosed herein. As appreciated by those skilled in the art, in some embodiments, sequences of oligonucleotides need not be 100% complementary to their targets for the oligonucleotides to perform their functions (e.g., skipping of a deleterious exon in an USH2A gene transcript). In some embodiments, homology, sequence identity or complementarity is 60%-100%, e.g., about or at least 60%, 70%, 75%, 80%, 85%, 90%, 91%, 92%, 93%, 94%, 95%, 96%, 97%, 98%, or 99%, or 100%. In some embodiments, a provided oligonucleotide has 75%-100% (e.g., about or at least 80%, 85%, 90%, 91%, 92%, 93%, 94%, 95%, 96%, 97%, 98%, or 99%, or 100%) sequence complementarity to a target region (e.g., a target sequence) within its target nucleic acid. In some embodiments, the percentage is about 80% or more. In some embodiments, the percentage is about 85% or more. In some embodiments, the percentage is about 90% or more. In some embodiments, the percentage is about 95% or more. For example, a provided oligonucleotide which is 20 nucleobases long will have 90 percent complementarity if 18 of its 20 nucleobases are complementary. Typically when determining complementarity, A and T (or U) are complementary nucleobases and C and G are complementary nucleobases.

In some embodiments, the present disclosure provides an USH2A oligonucleotide comprising a sequence found in an oligonucleotide described in a Table. In some embodiments, the present disclosure provides an USH2A oligonucleotide comprising a sequence found in an oligonucleotide described in a Table, wherein one or more U is independently and optionally replaced with T or vice versa. In some embodiments, an USH2A oligonucleotide can comprise at least one T and/or at least one U. In some embodiments, the present disclosure provides an USH2A oligonucleotide comprising a sequence found in an oligonucleotide described in a Table, wherein the said sequence has over 50% identity with the sequence of the oligonucleotide described in the Table. In some embodiments, the present disclosure provides an USH2A oligonucleotide comprising a sequence found in an oligonucleotide described in a Table, wherein the said sequence has over 60% identity with the sequence of the oligonucleotide described in the Table. In some embodiments, the present disclosure provides an USH2A oligonucleotide comprising a sequence found in an oligonucleotide described in a Table, wherein the said sequence has over 70% identity with the sequence of the oligonucleotide described in the Table. In some embodiments, the present disclosure provides an USH2A oligonucleotide comprising a sequence found in an oligonucleotide described in a Table, wherein the said sequence has over 80% identity with the sequence of the oligonucleotide described in the Table. In some embodiments, the present disclosure provides an USH2A oligonucleotide comprising a sequence found in an oligonucleotide described in a Table, wherein the said sequence has over 90% identity with the sequence of the oligonucleotide described in the Table. In some embodiments, the present disclosure provides an USH2A oligonucleotide comprising a sequence found in an oligonucleotide described in a Table, wherein the said sequence has over 95% identity with the sequence of the oligonucleotide described in the Table. In some embodiments, the present disclosure provides an USH2A oligonucleotide comprising the sequence of an oligonucleotide disclosed in a Table. In some embodiments, the present disclosure provides an USH2A oligonucleotide whose base sequence is the sequence of an oligonucleotide disclosed in a Table, wherein each U may be independently replaced with T and vice versa. In some embodiments, the present disclosure provides an USH2A oligonucleotide comprising a sequence found in an oligonucleotide in a Table, wherein the oligonucleotides have a pattern of backbone linkages, pattern of backbone chiral centers, and/or pattern of backbone phosphorus modifications of the same oligonucleotide or another oligonucleotide in a Table herein.

Among other things, the present disclosure presents, in Table A1 and elsewhere, various oligonucleotides, each of which has a defined base sequence. In some embodiments, the present disclosure, the present disclosure provides an oligonucleotide whose base sequence which is, comprises, or comprises a portion of the base sequence of an oligonucleotide disclosed herein, e.g., in a Table, e.g., Table A1 herein, wherein each U may be independently replaced with T and vice versa. In some embodiments, the disclosure provides an oligonucleotide having a base sequence which is, comprises, or comprises a portion of the base sequence of an oligonucleotide disclosed herein, e.g., in a Table, wherein each U may be independently replaced with T and vice versa, wherein the oligonucleotide further comprises a chemical modification, stereochemistry, format, an additional chemical moiety described herein (e.g., a targeting moiety, lipid moiety, carbohydrate moiety, etc.), and/or another structural feature.

In some embodiments, a “portion” (e.g., of a base sequence or a pattern of modifications) is at least 5, 6, 7, 8, 9, 10, 11, 12, 13, 14, 15, 16, 17, 18, 19, or 20 monomeric units long (e.g., for a base sequence, at least 5, 6, 7, 8, 9, 10, 11, 12, 13, 14, 15, 16, 17, 18, 19, or 20 bases long). In some embodiments, a “portion” of a base sequence is at least 5 bases long. In some embodiments, a “portion” of a base sequence is at least 10 bases long. In some embodiments, a “portion” of a base sequence is at least 15 bases long. In some embodiments, a “portion” of a base sequence is at least 20 bases long. In some embodiments, a portion of a base sequence is 10, 11, 12, 13, 14, 15, 16, 17, 18, 19 or more contiguous (consecutive) bases. In some embodiments, a portion of a base sequence is 15 or more contiguous (consecutive) bases.

In some embodiments, the present disclosure provides an oligonucleotide (e.g., an USH2A oligonucleotide) whose base sequence is a base sequence of an oligonucleotide in a Table or a portion thereof, wherein each U may be independently replaced with T and vice versa. In some embodiments, the present disclosure provides an USH2A oligonucleotide of a sequence of an oligonucleotide in a Table, wherein the oligonucleotide is capable of directing an increase in the level of skipping of a deleterious exon in an USH2A gene transcript or a gene product thereof. As appreciated by those skilled in the art, in provided base sequence, each U may be optionally and independently replaced by T or vice versa, and a sequence can comprise a mixture of U and T. In some embodiments, C may be optionally and independently replaced with 5mC.

In some embodiments, a portion is a span of at least 15, 16, 17, 18, 19, 20, 21, 22, 23, 24, or 25 total nucleotides. In some embodiments, a portion is a span of at least 15, 16, 17, 18, 19, 20, 21, 22, 23, 24, or 25 total nucleotides with 0-3 mismatches. In some embodiments, a portion is a span of at least 15, 16, 17, 18, 19, 20, 21, 22, 23, 24, or 25 total nucleotides with 0-3 mismatches, wherein a span with 0 mismatches is complementary and a span with 1 or more mismatches is a non-limiting example of substantial complementarity. In some embodiments, a base comprises a portion characteristic of a nucleic acid (e.g., a gene) in that the portion is identical or complementary to a portion of the nucleic acid or a transcript thereof, and is not identical or complementary to a portion of any other nucleic acid (e.g., a gene) or a transcript thereof in the same genome. In some embodiments, a portion is characteristic of human USH2A. In some embodiments, a portion is characteristic of human mUSH2A.

In some embodiments, a provided oligonucleotide, e.g., an USH2A oligonucleotide, has a length of no more than about 49, 45, 40, 30, 35, 25, or 23 total nucleotides as described herein. In some embodiments, wherein the sequence recited herein starts with a U or T at the 5′-end, the U can be deleted and/or replaced by another base. In some embodiments, an oligonucleotide has a base sequence which is or comprises or comprises a portion of the base sequence of an oligonucleotide in a Table, wherein each U may be independently replaced with T and vice versa, which has a format or a portion of a format disclosed herein.

In some embodiments, oligonucleotides, e.g., USH2A oligonucleotides are stereorandom. In some embodiments, oligonucleotides, e.g., USH2A oligonucleotides, are chirally controlled. In some embodiments, an USH2A oligonucleotide is chirally pure (or “stereopure”, “stereochemically pure”), wherein the oligonucleotide exists as a single stereoisomeric form (in many cases a single diastereoisomeric (or “diastereomeric”) form as multiple chiral centers may exist in an oligonucleotide, e.g., at linkage phosphorus, sugar carbon, etc.). As appreciated by those skilled in the art, a chirally pure oligonucleotide is separated from its other stereoisomeric forms (to the extent that some impurities may exist as chemical and biological processes, selectivities and/or purifications etc. rarely, if ever, go to absolute completeness). In a chirally pure oligonucleotide, each chiral center is independently defined with respect to its configuration (for a chirally pure oligonucleotide, each internucleotidic linkage is independently stereodefined or chirally controlled). In contrast to chirally controlled and chirally pure oligonucleotides which comprise stereodefined linkage phosphorus, racemic (or “stereorandom”, “non-chirally controlled”) oligonucleotides comprising chiral linkage phosphorus, e.g., from traditional phosphoramidite oligonucleotide synthesis without stereochemical control during coupling steps in combination with traditional sulfurization (creating stereorandom phosphorothioate internucleotidic linkages), refer to a random mixture of various stereoisomers (typically diastereoisomers (or “diastereomers”) as there are multiple chiral centers in an oligonucleotide; e.g., from traditional oligonucleotide preparation using reagents containing no chiral elements other than those in nucleosides and linkage phosphorus). For example, for A*A*A wherein * is a phosphorothioate internucleotidic linkage (which comprises a chiral linkage phosphorus), a racemic oligonucleotide preparation includes four diastereomers [2²=4, considering the two chiral linkage phosphorus, each of which can exist in either of two configurations (Sp or Rp)]: A *S A *S A, A *S A *R A, A *R A *S A, and A *R A *R A, wherein *S represents a Sp phosphorothioate internucleotidic linkage and *R represents a Rp phosphorothioate internucleotidic linkage. For a chirally pure oligonucleotide, e.g., A *S A *S A, it exists in a single stereoisomeric form and it is separated from the other stereoisomers (e.g., the diastereomers A *S A *R A, A *R A *S A, and A *R A *R A). In some embodiments, a Rp phosphorothioate is rendered as *S or * S. In some embodiments, a Rp phosphorothioate is rendered as *R or * R.

In some embodiments, oligonucleotides, e.g., USH2A oligonucleotides, comprise 1, 2, 3, 4, 5, 6, 7, 8, 9, 10 or more stereorandom internucleotidic linkages (mixture of Rp and Sp linkage phosphorus at the internucleotidic linkage, e.g., from traditional non-chirally controlled oligonucleotide synthesis). In some embodiments, oligonucleotides, e.g., USH2A oligonucleotides, comprise one or more (e.g., 1-50, 1-40, 1-30, 1-25, 1-20, 1, 2, 3, 4, 5, 6, 7, 8, 9, 10, 11, 12, 13, 14, 15, 16, 17, 18, 19, 20, 21, 22, 23, 24, 25 or more) chirally controlled internucleotidic linkages (Rp or Sp linkage phosphorus at the internucleotidic linkage, e.g., from chirally controlled oligonucleotide synthesis). In some embodiments, an internucleotidic linkage is a phosphorothioate internucleotidic linkage. In some embodiments, an internucleotidic linkage is a stereorandom phosphorothioate internucleotidic linkage. In some embodiments, an internucleotidic linkage is a chirally controlled phosphorothioate internucleotidic linkage.

Among other things, the present disclosure provides technologies for preparing chirally controlled (in some embodiments, stereochemically pure) oligonucleotides. In some embodiments, oligonucleotides are stereochemically pure. In some embodiments, oligonucleotides of the present disclosure are about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 15%, 20%, 25%, 30%, 35%, 40%, 45%, 50%, 55%, 60%, 65%, 70%, 75%, 80%, 85%, 90%, 95%, or 100%, or at least about 5%, 10%, 15%, 20%, 25%, 30%, 35%, 40%, 45%, 50%, 55%, 60%, 65%, 70%, 75%, 80%, 85%, 90%, 95%, or 99%, pure. In some embodiments, internucleotidic linkages of oligonucleotides comprise or consist of one or more (e.g., 1-50, 1-40, 1-30, 1-25, 1-20, 5-50, 5-40, 5-30, 5-25, 5-20, 1, 2, 3, 4, 5, 6, 7, 8, 9, 10, 11, 12, 13, 14, 15, 16, 17, 18, 19, 20, 21, 22, 23, 24, 25 or more) chiral internucleotidic linkages, each of which independently has a diastereopurity of at least 80%, 85%, 90%, 91%, 92%, 93%, 94%, 95%, 96%, 97%, 98%, 99% or 99.5%, typically at least 90%, 91%, 92%, 93%, 94%, 95%, 96%, 97%, 98%, 99% or 99.5%. In some embodiments, a chiral internucleotidic linkage has a diastereopurity of at least 95%. In some embodiments, a chiral internucleotidic linkage has a diastereopurity of at least 96%. In some embodiments, a chiral internucleotidic linkage has a diastereopurity of at least 97%. In some embodiments, a chiral internucleotidic linkage has a diastereopurity of at least 98%. In some embodiments, a chiral internucleotidic linkage has a diastereopurity of at least 99%. In some embodiments, oligonucleotides of the present disclosure, e.g., USH2A oligonucleotides, have a diastereopurity of (DS)^{CIL}, wherein DS is a diastereopurity as described in the present disclosure (e.g., 90%, 91%, 92%, 93%, 94%, 95%, 96%, 97%, 98%, 99% or 99.5% or more) and CIL is the number of chirally controlled internucleotidic linkages (e.g., 1-50, 1-40, 1-30, 1-25, 1-20, 5-50, 5-40, 5-30, 5-25, 5-20, 1, 2, 3, 4, 5, 6, 7, 8, 9, 10, 11, 12, 13, 14, 15, 16, 17, 18, 19, 20, 21, 22, 23, 24, 25 or more). In some embodiments, DS is 95%-100%. In some embodiments, each internucleotidic linkage is independently chirally controlled, and CIL is the number of chirally controlled internucleotidic linkages.

As examples, certain USH2A oligonucleotides comprising certain example base sequences, nucleobase modifications and patterns thereof, sugar modifications and patterns thereof, internucleotidic linkages and patterns thereof, linkage phosphorus stereochemistry and/or patterns thereof are presented in Table A1, below. Among other things, these oligonucleotides may be utilized to target an USH2A transcript, e.g., to mediate skipping of a deleterious exon in an USH2A gene transcript.

TABLE A1
Example USH2A Oligonucleotides.

| Oligo- | | | SEQ | |
|---|---|---|---|---|
| nucleo- | | | ID | Stereochemistry/ |
| tide | Description | Base Sequence | NO | Linkage |
| WV-20780 | mA * mG * mC * mU * mU * mC * mG * | AGCUUCGGAGAAAUUUA | 30 | XXXXX XXXXX |
| | mG * mA * G * mA * mA * mA * mU * m | AAUC | | XXXXX XXXXX |
| | U * mU * mA * mA * mA * mU * mC | | | |
| WV-20781 | Aco * Geo * m5Ceo * Teo * Teo * | AGCTTCGGAGAAATTTAA | 31 | XXXXX XXXXX |
| | m5Ceo * Geo * Geo * Aeo Geo * Aeo * | ATC | | XXXXX XXXXX |
| | Aeo * Aeo * Teo * Teo * Teo * Aeo * | | | |
| | Aeo * Aeo * Teo * m5Ceo | | | |
| WV-20782 | Aeo * Geo * m5Ceo * Teo * Teo * mC | AGCTTCGGAGAAAUUUA | 32 | XXXXX XXXXX |
| | * mG * mG * mA * mG * mA * mA * mA | AATC | | XXXXX XXXXX |
| | * mU * mU * mU * Aco * Aco * Aco * | | | |
| | Teo * m5Ceo | | | |
| WV-20783 | mA * mG * mC * mU * mUmCmGmGmAmGmAm | AGCUUCGGAGAAAUUUA | 33 | XXXXO OOOOO |
| | AmAmUmUmUmA * mA * mA * mU * mC | AAUC | | OOOOO OXXXX |
| WV-20784 | mA * mG * mC * mUmU * mCmG * mGmAmG | AGCUUCGGAGAAAUUUA | 34 | XXXOX OXOOO O |
| | mAmA * mAmU * mUmU * mAmA * mA * mU | AAUC | | XOXOX OXXX |
| | * mC | | | |
| WV-20879 | fC * SfU * SfA * SfA * SfA * SfG * | CUAAAGAUAAAAUAUAU | 35 | SSSSS SSSSS SSSSS |
| | SfA * SfU * SmA * SfA * SmA * SfA * | UUA | | SSSS |
| | SmU * SfA * SfU * SfA * SfU * SfU | | | |
| | * SfU * SfA | | | |
| WV-20880 | fA * SfA * SfG * SfC * SfC * SfC * | AAGCCCUAAAGAUAAAA | 36 | SSSSS SSSSS SSSSS |
| | SfU * SfA * SmA * SfA * SmG * SfA * | UAU | | SSSS |
| | SmU * SfA * SfA * SfA * SfA * SfU | | | |
| | * SfA * SfU | | | |
| WV-20881 | FC * SfA * SfC * SfC * SfU * SfA * | CACCUAAGCCCUAAAGA | 37 | SSSSS SSSSS SSSSS |
| | SfA * SfG * SmC * SfC * SmC * SfU * | UAA | | SSSS |
| | SmA * SfA * SfA * SfG * SfA * SfU | | | |
| | * SfA * SfA | | | |
| WV-20882 | fG * SfA * SfU * SfC * SfA * SfC * | GAUCACACCUAAGCCCU | 38 | SSSSS SSSSS SSSSS |
| | SfA * SfC * SmC * SfU * SmA * SfA * | AAA | | SSSS |
| | SmG * SfC * SfC * SfC * SfU * SfA | | | |
| | * SfA * SfA | | | |
| WV-20883 | fG * SfC * SfA * SfA * SfU * SfG * | GCAAUGAUCACACCUAA | 39 | SSSSS SSSSS SSSSS |
| | SfA * SfU * SmC * SfA * SmC * SfA * | GCC | | SSSS |
| | SmC * SfC * SfU * SfA * SfA * SfG | | | |
| | * SfC * SfC | | | |
| WV-20884 | fA * SfA * SfA * SfU * SfU * SfG * | AAAUUGCAAUGAUCACA | 40 | SSSSS SSSSS SSSSS |
| | SfC * SfA * SmA * SfU * SmG * SfA * | CCU | | SSSS |
| | SmU * SfC * SfA * SfC * SfA * SfC | | | |
| | * SfC * SfU | | | |
| WV-20885 | fA * SfU * SfC * SfC * SfA * SfA * | AUCCAAAAUUGCAAUGA | 41 | SSSSS SSSSS SSSSS |
| | SfA * SfA * SmU * SfU * SmG * SfC * | UCA | | SSSS |
| | SmA * SfA * SfU * SfG * SfA * SfU | | | |
| | * SfC * SfA | | | |
| WV-20886 | fU * SfU * SfU * SfA * SfA * SfA * | UUUAAAUCCAAAAUUGC | 42 | SSSSS SSSSS SSSSS |
| | SfU * SfC * SmC * SfA * SmA * SfA * | AAU | | SSSS |
| | SmA * SfU * SfU * SfG * SfC * SfA | | | |
| | * SfA * SfU | | | |
| WV-20887 | fA * SfG * SfA * SfA * SfA * SfU * | AGAAAUUUAAAUCCAAA | 43 | SSSSS SSSSS SSSSS |
| | SfU * SfU * SmA * SfA * SmA * SfU * | AUU | | SSSS |
| | SmC * SfC * SfA * SfA * SfA * SfA | | | |
| | * SfU * SfU | | | |
| WV-20888 | fU * SfU * SfC * SfG * SfG * SfA * | UUCGGAGAAAUUUAAAU | 44 | SSSSS SSSSS SSSSS |
| | SfG * SfA * SmA * SfA * SmU * SfU * | CCA | | SSSS |
| | SmU * SfA * SfA * SfA * SfU * SfC | | | |
| | * SfC * SfA | | | |
| WV-20889 | fA * SfA * SfA * SfG * SfC * SfU * | AAAGCUUCGGAGAAAUU | 45 | SSSSS SSSSS SSSSS |
| | SfU * SfC * SmG * SfG * SmA * SfG * | UAA | | SSSS |
| | SmA * SfA * SfA * SfU * SfU * SfU | | | |
| | * SfA * SfA | | | |
| WV-20890 | fU * SfC * SfA * SfU * SfU * SfA * | UCAUUAAAGCUUCGGAG | 46 | SSSSS SSSSS SSSSS |
| | SfA * SfA * SmG * SfC * SmU * SfU * | AAA | | SSSS |
| | SmC * SfG * SfG * SfA * SfG * SfA | | | |
| | * SfA * SfA | | | |
| WV-20891 | fC * SfA * SfA * SfC * SfA * SfU * | CAACAUCAUUAAAGCUU | 47 | SSSSS SSSSS SSSSS |
| | SfC * SfA * SmU * SfU * SmA * SfA * | CGG | | SSSS |
| | SmA * SfG * SfC * SfU * SfU * SfC | | | |
| | * SfG * SfG | | | |
| WV-20892 | fA * SfC * SfA * SfU * SfC * SfC * | ACAUCCAACAUCAUUAA | 48 | SSSSS SSSSS SSSSS |
| | SfA * SfA * SmC * SfA * SmU * SfC * | AGC | | SSSS |
| | SmA * SfU * SfU * SfA * SfA * SfA | | | |
| | * SfG * SfC | | | |
| WV-20893 | fG * SfG * SfC * SfU * SfC * SfA * | GGCUCACAUCCAACAUC | 49 | SSSSS SSSSS SSSSS |
| | SfC * SfA * SmU * SfC * SmC * SfA * | AUU | | SSSS |
| | SmA * SfC * SfA * SfU * SfC * SfA | | | |
| | * SfU * SfU | | | |
| WV-20894 | fG * SfG * SfC * SfA * SfG * SfG * | GGCAGGGCUCACAUCCA | 50 | SSSSS SSSSS SSSSS |
| | SfG * SfC * SmU * SfC * SmA * SfC * | ACA | | SSSS |
| | SmA * SfU * SfC * SfC * SfA * SfA | | | |
| | * SfC * SfA | | | |
| WV-20895 | fA * SfC * SfA * SfC * SfU * SfG * | ACACUGGCAGGGCUCAC | 51 | SSSSS SSSSS SSSSS |
| | SfG * SfC * SmA * SfG * SmG * SfG * | AUC | | SSSS |
| | SmC * SfU * SfC * SfA * SfC * SfA | | | |
| | * SfU * SfC | | | |
| WV-20896 | fA * SfG * SfG * SfU * SfU * SfA * | AGGUUACACUGGCAGGG | 52 | SSSSS SSSSS SSSSS |
| | SfC * SfA * SmC * SfU * SmG * SfG * | CUC | | SSSS |
| | SmC * SfA * SfG * SfG * SfG * SfC | | | |
| | * SfU * SfC | | | |
| WV-20897 | fC * SfA * SfU * SfG * SfG * SfA * | CAUGGAGGUUACACUGG | 53 | SSSSS SSSSS SSSSS |
| | SfG * SfG * SmU * SfU * SmA * SfC * | CAG | | SSSS |
| | SmA * SfC * SfU * SfG * SfG * SfC | | | |
| | * SfA * SfG | | | |
| WV-20898 | fU * SfG * SfA * SfG * SfC * SfC * | UGAGCCAUGGAGGUUAC | 54 | SSSSS SSSSS SSSSS |
| | SfA * SfU * SmG * SfG * SmA * SfG * | ACU | | SSSS |
| | SmG * SfU * SfU * SfA * SfC * SfA | | | |
| | * SfC * SfU | | | |
| WV-20899 | fU * SfU * SfC * SfA * SfC * SfU * | UUCACUGAGCCAUGGAG | 55 | SSSSS SSSSS SSSSS |
| | SfG * SfA * SmG * SfC * SmC * SfA * | GUU | | SSSS |
| | SmU * SfG * SfG * SfA * SfG * SfG | | | |
| | * SfU * SfU | | | |
| WV-20900 | fA * SfU * SfU * SfU * SfG * SfU * | AUUUGUUCACUGAGCCA | 56 | SSSSS SSSSS SSSSS |
| | SfU * SfC * SmA * SfC * SmU * SfG * | UGG | | SSSS |
| | SmA * SfG * SfC * SfC * SfA * SfU | | | |
| | * SfG * SfG | | | |
| WV-20901 | fG * SfC * SfA * SfG * SfA * SfA * | GCAGAAUUUGUUCACUG | 57 | SSSSS SSSSS SSSSS |
| | SfU * SfU * SmU * SfG * SmU * SfU * | AGC | | SSSS |
| | SmC * SfA * SfC * SfU * SfG * SfA | | | |
| | * SfG * SfC | | | |
| WV-20902 | fG * SfG * SfA * SfU * SfU * SfG * | GGAUUGCAGAAUUUGUU | 58 | SSSSS SSSSS SSSSS |
| | SfC * SfA * SmG * SfA * SmA * SfU * | CAC | | SSSS |
| | SmU * SfU * SfG * SfU * SfU * SfC | | | |
| | * SfA * SfC | | | |
| WV-20903 | fA * SfG * SfU * SfG * SfA * SfG * | AGUGAGGAUUGCAGAAU | 59 | SSSSS SSSSS SSSSS |
| | SfG * SfA * SmU * SfU * SmG * SfC * | UUG | | SSSS |
| | SmA * SfG * SfA * SfA * SfU * SfU | | | |
| | * SfU * SfG | | | |
| WV-20904 | fC * SfC * SfC * SfA * SfG * SfA * | CCCAGAGUGAGGAUUGC | 60 | SSSSS SSSSS SSSSS |
| | SfG * SfU * SmG * SfA * SmG * SfG * | AGA | | SSSS |
| | SmA * SfU * SfU * SfG * SfC * SfA | | | |
| | * SfG * SfA | | | |
| WV-20905 | fC * SfA * SfC * SfU * SfG * SfC * | CACUGCCCAGAGUGAGG | 61 | SSSSS SSSSS SSSSS |
| | SfC * SfC * SmA * SfG * SmA * SfG * | AUU | | SSSS |
| | SmU * SfG * SfA * SfG * SfG * SfA | | | |
| | * SfU * SfU | | | |
| WV-20906 | fA * SfC * SfU * SfC * SfA * SfC * | ACUCACACUGCCCAGAG | 62 | SSSSS SSSSS SSSSS |
| | SfA * SfC * SmU * SfG * SmC * SfC * | UGA | | SSSS |
| | SmC * SfA * SfG * SfA * SfG * SfU | | | |
| | * SfG * SfA | | | |
| WV-20907 | fU * SfU * SfU * SfG * SfC * SfA * | UUUGCACUCACACUGCC | 63 | SSSSS SSSSS SSSSS |
| | SfC * SfU * SmC * SfA * SmC * SfA * | CAG | | SSSS |
| | SmC * SfU * SfG * SfC * SfC * SfC | | | |
| | * SfA * SfG | | | |
| WV-20908 | fU * SfC * SfU * SfU * SfU * SfU * | UCUUUUUUGCACUCACA | 64 | SSSSS SSSSS SSSSS |
| | SfU * SfU * SmG * SfC * SmA * SfC * | CUG | | SSSS |
| | SmU * SfC * SfA * SfC * SfA * SfC | | | |
| | * SfU * SfG | | | |
| WV-20909 | fU * SfG * SfG * SfC * SfU * SfU * | UGGCUUCUUUUUUGCAC | 65 | SSSSS SSSSS SSSSS |
| | SfC * SfU * SmU * SfU * SmU * SfU * | UCA | | SSSS |
| | SmU * SfG * SfC * SfA * SfC * SfU | | | |
| | * SfC * SfA | | | |
| WV-20910 | fU * SfC * SfC * SfU * SfU * SfU * | UCCUUUGGCUUCUUUUU | 66 | SSSSS SSSSS SSSSS |
| | SfG * SfG * SmC * SfU * SmU * SfC * | UGC | | SSSS |
| | SmU * SfU * SfU * SfU * SfU * SfU | | | |
| | * SfG * SfC | | | |
| WV-20911 | fU * SfG * SfA * SfA * SfG * SfU * | UGAAGUCCUUUGGCUUC | 67 | SSSSS SSSSS SSSSS |
| | SfC * SfC * SmU * SfU * SmU * SfG * | UUU | | SSSS |
| | SmG * SfC * SfU * SfU * SfC * SfU | | | |
| | * SfU * SfU | | | |
| WV-20912 | fC * SfA * SfC * SfA * SfC * SfU * | CACACUGAAGUCCUUUG | 68 | SSSSS SSSSS SSSSS |
| | SfG * SfA * SmA * SfG * SmU * SfC * | GCU | | SSSS |
| | SmC * SfU * SfU * SfU * SfG * SfG | | | |
| | * SfC * SfU | | | |
| WV-20913 | fG * SfG * SfU * SfG * SfU * SfC * | GGUGUCACACUGAAGUC | 69 | SSSSS SSSSS SSSSS |
| | SfA * SfC * SmA * SfC * SmU * SfG * | CUU | | SSSS |
| | SmA * SfA * SfG * SfU * SfC * SfC | | | |
| | * SfU * SfU | | | |
| WV-20914 | fC * SfU * SfG * SfC * SfA * SfG * | CUGCAGGUGUCACACUG | 70 | SSSSS SSSSS SSSSS |
| | SfG * SfU * SmG * SfU * SmC * SfA * | AAG | | SSSS |
| | SmC * SfA * SfC * SfU * SfG * SfA | | | |
| | * SfA * SfG | | | |
| WV-20915 | fU * SfU * SfU * SfC * SfU * SfC * | UUUCUCUGCAGGUGUCA | 71 | SSSSS SSSSS SSSSS |
| | SfU * SfG * SmC * SfA * SmG * SfG * | CAC | | SSSS |
| | SmU * SfG * SfU * SfC * SfA * SfC | | | |
| | * SfA * SfC | | | |
| WV-20916 | fA * SfA * SfA * SfG * SfU * SfU * | AAAGUUUUCUCUGCAGG | 72 | SSSSS SSSSS SSSSS |
| | SfU * SfU * SmC * SfU * SmC * SfU * | UGU | | SSSS |
| | SmG * SfC * SfA * SfG * SfG * SfU | | | |
| | * SfG * SfU | | | |
| WV-20917 | fC * SfC * SfA * SfU * SfA * SfA * | CCAUAAAAGUUUUCUCU | 73 | SSSSS SSSSS SSSSS |
| | SfA * SfA * SmG * SfU * SmU * SfU * | GCA | | SSSS |
| | SmU * SfC * SfU * SfC * SfU * SfG | | | |
| | * SfC * SfA | | | |
| WV-20918 | fC * SfU * SfA * SfA * SfC * SfC * | CUAACCCAUAAAAGUUU | 74 | SSSSS SSSSS SSSSS |
| | SfC * SfA * SmU * SfA * SmA * SfA * | UCU | | SSSS |
| | SmA * SfG * SfU * SfU * SfU * SfU | | | |
| | * SfC * SfU | | | |
| WV-20919 | fG * SfA * SfC * SfA * SfU * SfC * | GACAUCUAACCCAUAAA | 75 | SSSSS SSSSS SSSSS |
| | SfU * SfA * SmA * SfC * SmC * SfC * | AGU | | SSSS |
| | SmA * SfU * SfA * SfA * SfA * SfA | | | |
| | * SfG * SfU | | | |
| WV-20920 | fU * SfU * SfG * SfG * SfU * SfG * | UUGGUGACAUCUAACCC | 76 | SSSSS SSSSS SSSSS |
| | SfA * SfC * SmA * SfU * SmC * SfU * | AUA | | SSSS |
| | SmA * SfA * SfC * SfC * SfC * SfA | | | |
| | * SfU * SfA | | | |
| WV-20921 | fU * SfA * SfC * SfA * SfA * SfU * | UACAAUUGGUGACAUCU | 77 | SSSSS SSSSS SSSSS |
| | SfU * SfG * SmG * SfU * SmG * SfA * | AAC | | SSSS |
| | SmC * SfA * SfU * SfC * SfU * SfA | | | |
| | * SfA * SfC | | | |
| WV-20922 | fG * SfG * SfC * SfC * SfU * SfU * | GGCCUUACAAUUGGUGA | 78 | SSSSS SSSSS SSSSS |
| | SfA * SfC * SmA * SfA * SmU * SfU * | CAU | | SSSS |
| | SmG * SfG * SfU * SfG * SfA * SfC | | | |
| | * SfA * SfU | | | |
| WV-20923 | fU * SfC * SfA * SfC * SfA * SfG * | UCACAGGCCUUACAAUU | 79 | SSSSS SSSSS SSSSS |
| | SfG * SfC * SmC * SfU * SmU * SfA * | GGU | | SSSS |
| | SmC * SfA * SfA * SfU * SfU * SfG | | | |
| | * SfG * SfU | | | |
| WV-20924 | fC * SfA * SfC * SfA * SfG * SfU * | CACAGUCACAGGCCUUA | 80 | SSSSS SSSSS SSSSS |
| | SfC * SfA * SmC * SfA * SmG * SfG * | CAA | | SSSS |
| | SmC * SfC * SfU * SfU * SfA * SfC | | | |
| | * SfA * SfA | | | |
| WV-20925 | fU * SfG * SfU * SfG * SfU * SfC * | UGUGUCACAGUCACAGG | 81 | SSSSS SSSSS SSSSS |
| | SfA * SfC * SmA * SfG * SmU * SfC * | CCU | | SSSS |
| | SmA * SfC * SfA * SfG * SfG * SfC | | | |
| | * SfC * SfU | | | |
| WV-20926 | fC * SfC * SfA * SfG * SfC * SfU * | CCAGCUGUGUCACAGUC | 82 | SSSSS SSSSS SSSSS |
| | SfG * SfU * SmG * SfU * SmC * SfA * | ACA | | SSSS |
| | SmC * SfA * SfG * SfU * SfC * SfA | | | |
| | * SfC * SfA | | | |
| WV-20927 | fG * SfG * SfG * SfA * SfU * SfC * | GGGAUCCAGCUGUGUCA | 83 | SSSSS SSSSS SSSSS |
| | SfC * SfA * SmG * SfC * SmU * SfG * | CAG | | SSSS |
| | SmU * SfG * SfU * SfC * SfA * SfC | | | |
| | * SfA * SfG | | | |
| WV-20928 | £A * SfG * SfG * SfG * SfA * SfG * | AGGGAGGGAUCCAGCUG | 84 | SSSSS SSSSS SSSSS |
| | SfG * SfG * SmA * SfU * SmC * SfC * | UGU | | SSSS |
| | SmA * SfG * SfC * SfU * SfG * SfU | | | |
| | * SfG * SfU | | | |
| WV-20929 | fG * SfU * SfC * SfC * SfC * SfA * | GUCCCAGGGAGGGAUCC | 85 | SSSSS SSSSS SSSSS |
| | SfG * SfG * SmG * SfA * SmG * SfG * | AGC | | SSSS |
| | SmG * SfA * SfU * SfC * SfC * SfA | | | |
| | * SfG * SfC | | | |
| WV-20930 | fA * SfG * SfA * SfC * SfA * SfG * | AGACAGUCCCAGGGAGG | 86 | SSSSS SSSSS SSSSS |
| | SfU * SfC * SmC * SfC * SmA * SfG * | GAU | | SSSS |
| | SmG * SfG * SfA * SfG * SfG * SfG | | | |
| | * SfA * SfU | | | |
| WV-20931 | fA * SfU * SfU * SfA * SfC * SfA * | AUUACAGACAGUCCCAG | 87 | SSSSS SSSSS SSSSS |
| | SfG * SfA * SmC * SfA * SmG * SfU * | GGA | | SSSS |
| | SmC * SfC * SfC * SfA * SfG * SfG | | | |
| | * SfG * SfA | | | |
| WV-20932 | fU * SfU * SfA * SfG * SfC * SfA * | UUAGCAUUACAGACAGU | 88 | SSSSS SSSSS SSSSS |
| | SfU * SfU * SmA * SfC * SmA * SfG * | CCC | | SSSS |
| | SmA * SfC * SfA * SfG * SfU * SfC | | | |
| | * SfC * SfC | | | |
| WV-20933 | fC * SfU * SfG * SfU * SfC * SfU * | CUGUCUUAGCAUUACAG | 89 | SSSSS SSSSS SSSSS |
| | SfU * SfA * SmG * SfC * SmA * SfU * | ACA | | SSSS |
| | SmU * SfA * SfC * SfA * SfG * SfA | | | |
| | * SfC * SfA | | | |
| WV-20934 | fC * SfU * SfG * SfC * SfC * SfC * | CUGCCCUGUCUUAGCAU | 90 | SSSSS SSSSS SSSSS |
| | SfU * SfG * SmU * SfC * SmU * SfU * | UAC | | SSSS |
| | SmA * SfG * SfC * SfA * SfU * SfU | | | |
| | * SfA * SfC | | | |
| WV-20935 | fA * SfU * SfG * SfC * SfA * SfC * | AUGCACUGCCCUGUCUU | 91 | SSSSS SSSSS SSSSS |
| | SfU * SfG * SmC * SfC * SmC * SfU * | AGC | | SSSS |
| | SmG * SfU * SfC * SfU * SfU * SfA | | | |
| | * SfG * SfC | | | |
| WV-20936 | fU * SfG * SfC * SfA * SfG * SfA * | UGCAGAUGCACUGCCCU | 92 | SSSSS SSSSS SSSSS |
| | SfU * SfG * SmC * SfA * SmC * SfU * | GUC | | SSSS |
| | SmG * SfC * SfC * SfC * SfU * SfG | | | |
| | * SfU * SfC | | | |
| WV-20937 | fG * SfG * SfG * SfC * SfU * SfU * | GGGCUUGCAGAUGCACU | 93 | SSSSS SSSSS SSSSS |
| | SfG * SfC * SmA * SfG * SmA * SfU * | GCC | | SSSS |
| | SmG * SfC * SfA * SfC * SfU * SfG | | | |
| | * SfC * SfC | | | |
| WV-20938 | fA * SfC * SfA * SfU * SfU * SfG * | ACAUUGGGCUUGCAGAU | 94 | SSSSS SSSSS SSSSS |
| | SfG * SfG * SmC * SfU * SmU * SfG * | GCA | | SSSS |
| | SmC * SfA * SfG * SfA * SfU * SfG | | | |
| | * SfC * SfA | | | |
| WV-20939 | fC * SfU * SfU * SfC * SfA * SfA * | CUUCAACAUUGGGCUUG | 95 | SSSSS SSSSS SSSSS |
| | SfC * SfA * SmU * SfU * SmG * SfG * | CAG | | SSSS |
| | SmG * SfC * SfU * SfU * SfG * SfC | | | |
| | * SfA * SfG | | | |
| WV-20940 | fU * SfC * SfU * SfC * SfC * SfC * | UCUCCCUUCAACAUUGG | 96 | SSSSS SSSSS SSSSS |
| | SfU * SfU * SmC * SfA * SmA * SfC * | GCU | | SSSS |
| | SmA * SfU * SfU * SfG * SfG * SfG | | | |
| | * SfC * SfU | | | |
| WV-20941 | fC * SfA * SfC * SfU * SfG * SfU * | CACUGUCUCCCUUCAAC | 97 | SSSSS SSSSS SSSSS |
| | SfC * SfU * SmC * SfC * SmC * SfU * | AUU | | SSSS |
| | SmU * SfC * SfA * SfA * SfC * SfA | | | |
| | * SfU * SfU | | | |
| WV-20942 | fU * SfA * SfU * SfU * SfG * SfC * | UAUUGCACUGUCUCCCU | 98 | SSSSS SSSSS SSSSS |
| | SfA * SfC * SmU * SfG * SmU * SfC * | UCA | | SSSS |
| | SmU * SfC * SfC * SfC * SfU * SfU | | | |
| | * SfC * SfA | | | |
| WV-20943 | £A * SfC * SfA * SfU * SfU * SfU * | ACAUUUAUUGCACUGUC | 99 | SSSSS SSSSS SSSSS |
| | SfA * SfU * SmU * SfG * SmC * SfA * | UCC | | SSSS |
| | SmC * SfU * SfG * SfU * SfC * SfU | | | |
| | * SfC * SfC | | | |
| WV-20944 | fU * SfC * SfC * SfA * SfA * SfA * | UCCAAACAUUUAUUGCA | 100 | SSSSS SSSSS SSSSS |
| | SfC * SfA * SmU * SfU * SmU * SfA * | CUG | | SSSS |
| | SmU * SfU * SfG * SfC * SfA * SfC | | | |
| | * SfU * SfG | | | |
| WV-20945 | fU * SfU * SfC * SfC * SfC * SfU * | UUCCCUCCAAACAUUUA | 101 | SSSSS SSSSS SSSSS |
| | SfC * SfC * SmA * SfA * SmA * SfC * | UUG | | SSSS |
| | SmA * SfU * SfU * SfU * SfA * SfU | | | |
| | * SfU * SfG | | | |
| WV-20946 | fG * SfA * SfA * SfG * SfU * SfU * | GAAGUUUCCCUCCAAAC | 102 | SSSSS SSSSS SSSSS |
| | SfU * SfC * SmC * SfC * SmU * SfC * | AUU | | SSSS |
| | SmC * SfA * SfA * SfA * SfC * SfA | | | |
| | * SfU * SfU | | | |
| WV-20947 | fA * SfG * SfG * SfU * SfA * SfG * | AGGUAGAAGUUUCCCUC | 103 | SSSSS SSSSS SSSSS |
| | SfA * SfA * SmG * SfU * SmU * SfU * | CAA | | SSSS |
| | SmC * SfC * SfC * SfU * SfC * SfC | | | |
| | * SfA * SfA | | | |
| WV-20948 | fG * SfC * SfC * SfG * SfU * SfA * | GCCGUAGGUAGAAGUUU | 104 | SSSSS SSSSS SSSSS |
| | SfG * SfG * SmU * SfA * SmG * SfA * | CCC | | SSSS |
| | SmA * SfG * SfU * SfU * SfU * SfC | | | |
| | * SfC * SfC | | | |
| WV-20949 | fA * SfU * SfU * SfU * SfU * SfG * | AUUUUGCCGUAGGUAGA | 105 | SSSSS SSSSS SSSSS |
| | SfC * SfC * SmG * SfU * SmA * SfG * | AGU | | SSSS |
| | SmG * SfU * SfA * SfG * SfA * SfA | | | |
| | * SfG * SfU | | | |
| WV-20950 | fG * SfA * SfA * SfU * SfU * SfA * | GAAUUAUUUUGCCGUAG | 106 | SSSSS SSSSS SSSSS |
| | SfU * SfU * SmU * SfU * SmG * SfC * | GUA | | SSSS |
| | SmC * SfG * SfU * SfA * SfG * SfG | | | |
| | * SfU * SfA | | | |
| WV-20951 | fG * SfG * SfA * SfA * SfA * SfG * | GGAAAGAAUUAUUUUGC | 107 | SSSSS SSSSS SSSSS |
| | SfA * SfA * SmU * SfU * SmA * SfU * | CGU | | SSSS |
| | SmU * SfU * SfU * SfG * SfC * SfC | | | |
| | * SfG * SfU | | | |
| WV-20952 | fA * SfC * SfA * SfG * SfA * SfG * | ACAGAGGAAAGAAUUAU | 108 | SSSSS SSSSS SSSSS |
| | SfG * SfA * SmA * SfA * SmG * SfA * | UUU | | SSSS |
| | SmA * SfU * SfU * SfA * SfU * SfU | | | |
| | * SfU * SfU | | | |
| WV-20953 | fG * SfG * SfC * SfA * SfG * SfA * | GGCAGACAGAGGAAAGA | 109 | SSSSS SSSSS SSSSS |
| | SfC * SfA * SmG * SfA * SmG * SfG * | AUU | | SSSS |
| | SmA * SfA * SfA * SfG * SfA * SfA | | | |
| | * SfU * SfU | | | |
| WV-20954 | fU * SfG * SfC * SfA * SfA * SfG * | UGCAAGGCAGACAGAGG | 110 | SSSSS SSSSS SSSSS |
| | SfG * SfC * SmA * SfG * SmA * SfC * | AAA | | SSSS |
| | SmA * SfG * SfA * SfG * SfG * SfA | | | |
| | * SfA * SfA | | | |
| WV-20955 | fA * SfC * SfA * SfG * SfU * SfU * | ACAGUUGCAAGGCAGAC | 111 | SSSSS SSSSS SSSSS |
| | SfG * SfC * SmA * SfA * SmG * SfG * | AGA | | SSSS |
| | SmC * SfA * SfG * SfA * SfC * SfA | | | |
| | * SfG * SfA | | | |
| WV-20956 | fU * SfU * SfA * SfU * SfC * SfA * | UUAUCACAGUUGCAAGG | 112 | SSSSS SSSSS SSSSS |
| | SfC * SfA * SmG * SfU * SmU * SfG * | CAG | | SSSS |
| | SmC * SfA * SfA * SfG * SfG * SfC | | | |
| | * SfA * SfG | | | |
| WV-20957 | fC * SfA * SfG * SfU * SfC * SfU * | CAGUCUUAUCACAGUUG | 113 | SSSSS SSSSS SSSSS |
| | SfU * SfA * SmU * SfC * SmA * SfC * | CAA | | SSSS |
| | SmA * SfG * SfU * SfU * SfG * SfC | | | |
| | * SfA * SfA | | | |
| WV-20958 | fU * SfG * SfU * SfC * SfC * SfC * | UGUCCCAGUCUUAUCAC | 114 | SSSSS SSSSS SSSSS |
| | SfA * SfG * SmU * SfC * SmU * SfU * | AGU | | SSSS |
| | SmA * SfU * SfC * SfA * SfC * SfA | | | |
| | * SfG * SfU | | | |
| WV-20959 | fU * SfU * SfU * SfA * SfU * SfU * | UUUAUUGUCCCAGUCUU | 115 | SSSSS SSSSS SSSSS |
| | SfG * SfU * SmC * SfC * SmC * SfA * | AUC | | SSSS |
| | SmG * SfU * SfC * SfU * SfU * SfA | | | |
| | * SfU * SfC | | | |
| WV-20960 | fA * SfG * SfC * SfC * SfA * SfU * | AGCCAUUUAUUGUCCCA | 116 | SSSSS SSSSS SSSSS |
| | SfU * SfU * SmA * SfU * SmU * SfG * | GUC | | SSSS |
| | SmU * SfC * SfC * SfC * SfA * SfG | | | |
| | * SfU * SfC | | | |
| WV-20961 | fC * SfA * SfG * SfA * SfG * SfA * | CAGAGAGCCAUUUAUUG | 117 | SSSSS SSSSS SSSSS |
| | SfG * SfC * SmC * SfA * SmU * SfU * | UCC | | SSSS |
| | SmU * SfA * SfU * SfU * SfG * SfU | | | |
| | * SfC * SfC | | | |
| WV-20962 | fC * SfA * SfC * SfA * SfG * SfC * | CACAGCAGAGAGCCAUU | 118 | SSSSS SSSSS SSSSS |
| | SfA * SfG * SmA * SfG * SmA * SfG * | UAU | | SSSS |
| | SmC * SfC * SfA * SfU * SfU * SfU | | | |
| | * SfA * SfU | | | |
| WV-20963 | fU * SfG * SfU * SfU * SfA * SfC * | UGUUACACAGCAGAGAG | 119 | SSSSS SSSSS SSSSS |
| | SfA * SfC * SmA * SfG * SmC * SfA * | CCA | | SSSS |
| | SmG * SfA * SfG * SfA * SfG * SfC | | | |
| | * SfC * SfA | | | |
| WV-20964 | fU * SfG * SfA * SfU * SfU * SfU * | UGAUUUGUUACACAGCA | 120 | SSSSS SSSSS SSSSS |
| | SfG * SfU * SmU * SfA * SmC * SfA * | GAG | | SSSS |
| | SmC * SfA * SfG * SfC * SfA * SfG | | | |
| | * SfA * SfG | | | |
| WV-20965 | fC * SfC * SfU * SfG * SfU * SfU * | CCUGUUGAUUUGUUACA | 121 | SSSSS SSSSS SSSSS |
| | SfG * SfA * SmU * SfU * SmU * SfG * | CAG | | SSSS |
| | SmU * SfU * SfA * SfC * SfA * SfC | | | |
| | * SfA * SfG | | | |
| WV-20966 | fA * SfU * SfU * SfG * SfU * SfC * | AUUGUCCUGUUGAUUUG | 122 | SSSSS SSSSS SSSSS |
| | SfC * SfU * SmG * SfU * SmU * SfG * | UUA | | SSSS |
| | SmA * SfU * SfU * SfU * SfG * SfU | | | |
| | * SfU * SfA | | | |
| WV-20967 | fA * SfG * SfG * SfA * SfC * SfA * | AGGACAUUGUCCUGUUG | 123 | SSSSS SSSSS SSSSS |
| | SfU * SfU * SmG * SfU * SmC * SfC * | AUU | | SSSS |
| | SmU * SfG * SfU * SfU * SfG * SfA | | | |
| | * SfU * SfU | | | |
| WV-20968 | fU * SfU * SfG * SfC * SfA * SfA * | UUGCAAGGACAUUGUCC | 124 | SSSSS SSSSS SSSSS |
| | SfG * SfG * SmA * SfC * SmA * SfU * | UGU | | SSSS |
| | SmU * SfG * SfU * SfC * SfC * SfU | | | |
| | * SfG * SfU | | | |
| WV-20969 | fC * SfU * SfA * SfA * SfU * SfU * | CUAAUUUGCAAGGACAU | 125 | SSSSS SSSSS SSSSS |
| | SfU * SfG * SmC * SfA * SmA * SfG * | UGU | | SSSS |
| | SmG * SfA * SfC * SfA * SfU * SfU | | | |
| | * SfG * SfU | | | |
| WV-20970 | fU * SfA * SfC * SfC * SfC * SfC * | UACCCCUAAUUUGCAAG | 126 | SSSSS SSSSS SSSSS |
| | SfU * SfA * SmA * SfU * SmU * SfU * | GAC | | SSSS |
| | SmG * SfC * SfA * SfA * SfG * SfG | | | |
| | * SfA * SfC | | | |
| WV-20971 | fC * SfC * SfU * SfG * SfU * SfU * | CCUGUUACCCCUAAUUU | 127 | SSSSS SSSSS SSSSS |
| | SfA * SfC * SmC * SfC * SmC * SfU * | GCA | | SSSS |
| | SmA * SfA * SfU * SfU * SfU * SfG | | | |
| | * SfC * SfA | | | |
| WV-20972 | fG * SfA * SfA * SfG * SfA * SfC * | GAAGACCUGUUACCCCU | 128 | SSSSS SSSSS SSSSS |
| | SfC * SfU * SmG * SfU * SmU * SfA * | AAU | | SSSS |
| | SmC * SfC * SfC * SfC * SfU * SfA | | | |
| | * SfA * SfU | | | |
| WV-20973 | fA * SfC * SfA * SfG * SfC * SfG * | ACAGCGAAGACCUGUUA | 129 | SSSSS SSSSS SSSSS |
| | SfA * SfA * SmG * SfA * SmC * SfC * | CCC | | SSSS |
| | SmU * SfG * SfU * SfU * SfA * SfC | | | |
| | * SfC * SfC | | | |
| WV-20974 | fU * SfG * SfA * SfU * SfU * SfA * | UGAUUACAGCGAAGACC | 130 | SSSSS SSSSS SSSSS |
| | SfC * SfA * SmG * SfC * SmG * SfA * | UGU | | SSSS |
| | SmA * SfG * SfA * SfC * SfC * SfU | | | |
| | * SfG * SfU | | | |
| WV-20975 | fC * SfA * SfC * SfA * SfC * SfU * | CACACUGAUUACAGCGA | 131 | SSSSS SSSSS SSSSS |
| | SfG * SfA * SmU * SfU * SmA * SfC * | AGA | | SSSS |
| | SmA * SfG * SfC * SfG * SfA * SfA | | | |
| | * SfG * SfA | | | |
| WV-20976 | fA * SfG * SfG * SfC * SfU * SfC * | AGGCUCACACUGAUUAC | 132 | SSSSS SSSSS SSSSS |
| | SfA * SfC * SmA * SfC * SmU * SfG * | AGC | | SSSS |
| | SmA * SfU * SfU * SfA * SfC * SfA | | | |
| | * SfG * SfC | | | |
| WV-20977 | fC * SfU * SfG * SfU * SfG * SfA * | CUGUGAGGCUCACACUG | 133 | SSSSS SSSSS SSSSS |
| | SfG * SfG * SmC * SfU * SmC * SfA * | AUU | | SSSS |
| | SmC * SfA * SfC * SfU * SfG * SfA | | | |
| | * SfU * SfU | | | |
| WV-20978 | fU * SfG * SfU * SfA * SfC * SfC * | UGUACCUGUGAGGCUCA | 134 | SSSSS SSSSS SSSSS |
| | SfU * SfG * SmU * SfG * SmA * SfG * | CAC | | SSSS |
| | SmG * SfC * SfU * SfC * SfA * SfC | | | |
| | * SfA * SfC | | | |
| WV-20979 | fC * SfA * SfA * SfA * SfU * SfU * | CAAAUUGUACCUGUGAG | 135 | SSSSS SSSSS SSSSS |
| | SfG * SfU * SmA * SfC * SmC * SfU * | GCU | | SSSS |
| | SmG * SfU * SfG * SfA * SfG * SfG | | | |
| | * SfC * SfU | | | |
| WV-20980 | fA * SfU * SfG * SfG * SfU * SfC * | AUGGUCAAAUUGUACCU | 136 | SSSSS SSSSS SSSSS |
| | SfA * SfA * SmA * SfU * SmU * SfG * | GUG | | SSSS |
| | SmU * SfA * SfC * SfC * SfU * SfG | | | |
| | * SfU * SfG | | | |
| WV-20981 | fU * SfG * SfU * SfC * SfA * SfA * | UGUCAAUGGUCAAAUUG | 137 | SSSSS SSSSS SSSSS |
| | SfU * SfG * SmG * SfU * SmC * SfA * | UAC | | SSSS |
| | SmA * SfA * SfU * SfU * SfG * SfU | | | |
| | * SfA * SfC | | | |
| WV-20982 | fA * SfA * SfA * SfA * SfU * SfU * | AAAAUUGUCAAUGGUCA | 138 | SSSSS SSSSS SSSSS |
| | SfG * SfU * SmC * SfA * SmA * SfU * | AAU | | SSSS |
| | SmG * SfG * SfU * SfC * SfA * SfA | | | |
| | * SfA * SfU | | | |
| WV-20983 | fU * SfG * SfU * SfU * SfG * SfA * | UGUUGAAAAUUGUCAAU | 139 | SSSSS SSSSS SSSSS |
| | SfA * SfA * SmA * SfU * SmU * SfG * | GGU | | SSSS |
| | SmU * SfC * SfA * SfA * SfU * SfG | | | |
| | * SfG * SfU | | | |
| WV-20984 | fG * SfG * SfC * SfA * SfG * SfU * | GGCAGUGUUGAAAAUUG | 140 | SSSSS SSSSS SSSSS |
| | SfG * SfU * SmU * SfG * SmA * SfA * | UCA | | SSSS |
| | SmA * SfA * SfU * SfU * SfG * SfU | | | |
| | * SfC * SfA | | | |
| WV-20985 | fC * SfA * SfU * SfC * SfU * SfG * | CAUCUGGCAGUGUUGAA | 141 | SSSSS SSSSS SSSSS |
| | SfG * SfC * SmA * SfG * SmU * SfG * | AAU | | SSSS |
| | SmU * SfU * SfG * SfA * SfA * SfA | | | |
| | * SfA * SfU | | | |
| WV-20986 | fU * SfC * SfA * SfC * SfA * SfC * | UCACACAUCUGGCAGUG | 142 | SSSSS SSSSS SSSSS |
| | SfA * SfU * SmC * SfU * SmG * SfG * | UUG | | SSSS |
| | SmC * SfA * SfG * SfU * SfG * SfU | | | |
| | * SfU * SfG | | | |
| WV-20987 | fC * SfA * SfC * SfA * SfC * SfU * | CACACUCACACAUCUGG | 143 | SSSSS SSSSS SSSSS |
| | SfC * SfA * SmC * SfA * SmC * SfA * | CAG | | SSSS |
| | SmU * SfC * SfU * SfG * SfG * SfC | | | |
| | * SfA * SfG | | | |
| WV-20988 | fG * SfG * SfA * SfA * SfU * SfC * | GGAAUCACACUCACACA | 144 | SSSSS SSSSS SSSSS |
| | SfA * SfC * SmA * SfC * SmU * SfC * | UCU | | SSSS |
| | SmA * SfC * SfA * SfC * SfA * SfU | | | |
| | * SfC * SfU | | | |
| WV-20989 | fC * SfC * SfC * SfA * SfA * SfG * | CCCAAGGAAUCACACUC | 145 | SSSSS SSSSS SSSSS |
| | SfG * SfA * SmA * SfU * SmC * SfA * | ACA | | SSSS |
| | SmC * SfA * SfC * SfU * SfC * SfA | | | |
| | * SfC * SfA | | | |
| WV-20990 | fA * SfU * SfG * SfU * SfC * SfC * | AUGUCCCCAAGGAAUCA | 146 | SSSSS SSSSS SSSSS |
| | SfC * SfC * SmA * SfA * SmG * SfG * | CAC | | SSSS |
| | SmA * SfA * SfU * SfC * SfA * SfC | | | |
| | * SfA * SfC | | | |
| WV-20991 | fA * SfG * SfG * SfU * SfA * SfA * | AGGUAAUGUCCCCAAGG | 147 | SSSSS SSSSS SSSSS |
| | SfU * SfG * SmU * SfC * SmC * SfC * | AAU | | SSSS |
| | SmC * SfA * SfA * SfG * SfG * SfA | | | |
| | * SfA * SfU | | | |
| WV-20992 | fG * SfU * SfC * SfC * SfC * SfA * | GUCCCAGGUAAUGUCCC | 148 | SSSSS SSSSS SSSSS |
| | SfG * SfG * SmU * SfA * SmA * SfU * | CAA | | SSSS |
| | SmG * SfU * SfC * SfC * SfC * SfC | | | |
| | * SfA * SfA | | | |
| WV-20993 | fA * SfA * SfA * SfU * SfG * SfG * | AAAUGGUCCCAGGUAAU | 149 | SSSSS SSSSS SSSSS |
| | SfU * SfC * SmC * SfC * SmA * SfG * | GUC | | SSSS |
| | SmG * SfU * SfA * SfA * SfU * SfG | | | |
| | * SfU * SfC | | | |
| WV-20994 | fG * SfU * SfC * SfA * SfC * SfA * | GUCACAAAUGGUCCCAG | 150 | SSSSS SSSSS SSSSS |
| | SfA * SfA * SmU * SfG * SmG * SfU * | GUA | | SSSS |
| | SmC * SfC * SfC * SfA * SfG * SfG | | | |
| | * SfU * SfA | | | |
| WV-20995 | fA * SfU * SfU * SfG * SfG * SfG * | AUUGGGUCACAAAUGGU | 151 | SSSSS SSSSS SSSSS |
| | SfU * SfC * SmA * SfC * SmA * SfA * | CCC | | SSSS |
| | SmA * SfU * SfG * SfG * SfU * SfC | | | |
| | * SfC * SfC | | | |
| WV-20996 | fC * SfA * SfC * SfU * SfG * SfA * | CACUGAUUGGGUCACAA | 152 | SSSSS SSSSS SSSSS |
| | SfU * SfU * SmG * SfG * SmG * SfU * | AUG | | SSSS |
| | SmC * SfA * SfC * SfA * SfA * SfA | | | |
| | * SfU * SfG | | | |
| WV-20997 | fC * SfU * SfG * SfG * SfC * SfC * | CUGGCCACUGAUUGGGU | 153 | SSSSS SSSSS SSSSS |
| | SfA * SfC * SmU * SfG * SmA * SfU * | CAC | | SSSS |
| | SmU * SfG * SfG * SfG * SfU * SfC | | | |
| | * SfA * SfC | | | |
| WV-20998 | fA * SfG * SfG * SfC * SfA * SfC * | AGGCACUGGCCACUGAU | 154 | SSSSS SSSSS SSSSS |
| | SfU * SfG * SmG * SfC * SmC * SfA * | UGG | | SSSS |
| | SmC * SfU * SfG * SfA * SfU * SfU | | | |
| | * SfG * SfG | | | |
| WV-20999 | fC * SfA * SfC * SfA * SfC * SfA * | CACACAGGCACUGGCCA | 155 | SSSSS SSSSS SSSSS |
| | SfG * SfG * SmC * SfA * SmC * SfU * | CUG | | SSSS |
| | SmG * SfG * SfC * SfC * SfA * SfC | | | |
| | * SfU * SfG | | | |
| WV-21000 | fA * SfG * SfG * SfC * SfA * SfC * | AGGCACACACAGGCACU | 156 | SSSSS SSSSS SSSSS |
| | SfA * SfC * SmA * SfC * SmA * SfG * | GGC | | SSSS |
| | SmG * SfC * SfA * SfC * SfU * SfG | | | |
| | * SfG * SfC | | | |
| WV-21001 | fC * SfG * SfA * SfU * SfU * SfA * | CGAUUAGGCACACACAG | 157 | SSSSS SSSSS SSSSS |
| | SfG * SfG * SmC * SfA * SmC * SfA * | GCA | | SSSS |
| | SmC * SfA * SfC * SfA * SfG * SfG | | | |
| | * SfC * SA | | | |
| WV-21002 | fC * SfU * SfU * SfG * SfA * SfC * | CUUGACGAUUAGGCACA | 158 | SSSSS SSSSS SSSSS |
| | SfG * SfA * SmU * SfU * SmA * SfG * | CAC | | SSSS |
| | SmG * SfC * SfA * SfC * SfA * SfC | | | |
| | * SfA * SfC | | | |
| WV-21003 | fU * SfC * SfU * SfU * SfC * SfC * | UCUUCCUUGACGAUUAG | 159 | SSSSS SSSSS SSSSS |
| | SfU * SfU * SmG * SfA * SmC * SfG * | GCA | | SSSS |
| | SmA * SfU * SfU * SfA * SfG * SfG | | | |
| | * SfC * SfA | | | |
| WV-21004 | fC * SfA * SfC * SfC * SfU * SfU * | CACCUUCUUCCUUGACG | 160 | SSSSS SSSSS SSSSS |
| | SfC * SfU * SmU * SfC * SmC * SfU * | AUU | | SSSS |
| | SmU * SfG * SfA * SfC * SfG * SfA | | | |
| | * SfU * SfU | | | |
| WV-21005 | fG * SfA * SfU * SfU * SfA * SfC * | GAUUACACCUUCUUCCU | 161 | SSSSS SSSSS SSSSS |
| | SfA * SfC * SmC * SfU * SmU * SfC * | UGA | | SSSS |
| | SmU * SfU * SfC * SfC * SfU * SfU | | | |
| | * SfG * SfA | | | |
| WV-21006 | fA * SfC * SfA * SfC * SfU * SfG * | ACACUGAUUACACCUUC | 162 | SSSSS SSSSS SSSSS |
| | SfA * SfU * SmU * SfA * SmC * SfA * | UUC | | SSSS |
| | SmC * SfC * SfU * SfU * SfC * SfU | | | |
| | * SfU * SfC | | | |
| WV-21007 | fG * SfG * SfU * SfU * SfG * SfA * | GGUUGACACUGAUUACA | 163 | SSSSS SSSSS SSSSS |
| | SfC * SfA * SmC * SfU * SmG * SfA * | CCU | | SSSS |
| | SmU * SfU * SfA * SfC * SfA * SfC | | | |
| | * SfC * SfU | | | |
| WV-21008 | fU * SfA * SfC * SfC * SfU * SfG * | UACCUGGUUGACACUGA | 164 | SSSSS SSSSS SSSSS |
| | SfG * SfU * SmU * SfG * SmA * SfC * | UUA | | SSSS |
| | SmA * SfC * SfU * SfG * SfA * SfU | | | |
| | * SfU * SfA | | | |
| WV-21009 | fU * SfU * SfU * SfC * SfU * SfU * | UUUCUUACCUGGUUGAC | 165 | SSSSS SSSSS SSSSS |
| | SfA * SfC * SmC * SfU * SmG * SfG * | ACU | | SSSS |
| | SmU * SfU * SfG * SfA * SfC * SfA | | | |
| | * SfC * SfU | | | |
| WV-21010 | fA * SfU * SfU * SfU * SfC * SfU * | AUUUCUUUCUUACCUGG | 166 | SSSSS SSSSS SSSSS |
| | SfU * SfU * SmC * SfU * SmU * SfA * | UUG | | SSSS |
| | SmC * SfC * SfU * SfG * SfG * SfU | | | |
| | * SfU * SfG | | | |
| WV-21011 | fA * SfA * SfU * SfA * SfC * SfA * | AAUACAUUUCUUUCUUA | 167 | SSSSS SSSSS SSSSS |
| | SfU * SfU * SmU * SfC * SmU * SfU * | CCU | | SSSS |
| | SmU * SfC * SfU * SfU * SfA * SfC | | | |
| | * SfC * SfU | | | |
| WV-21012 | fG * SfU * SfA * SfA * SfU * SfA * | GUAAUACAUUUCUUUCU | 168 | SSSSS SSSSS SSSSS |
| | SfC * SfA * SmU * SfU * SmU * SfC * | UAC | | SSSS |
| | SmU * SfU * SfU * SfC * SfU * SfU | | | |
| | * SfA * SfC | | | |
| WV-21094 | fA * SfG * SfC * SfU * SfU * SfC * | AGCUUCGGAGAAAUUUA | 169 | SSSSS SSSSS SSSSS |
| | SfG * SfG * SmA * SfG * SmA * SfA * | AAUC | | SSSSS |
| | SmA * SfU * SfU * SfU * SfA * SfA | | | |
| | * SfA * SfU * SfC | | | |
| WV-21095 | fA * SfG * SfC * SfU * SfU * SfC * | AGCUUCGGAGAAAUUUA | 170 | SSSSS SSSSS SSSSS |
| | SfG * SfG * SmA * SfG * SmA * SfA * | AAU | | SSSS |
| | SmA * SfU * SfU * SfU * SfA * SfA | | | |
| | * SfA * SfU | | | |
| WV-21096 | fG * SfC * SfU * SfU * SfC * SfG * | GCUUCGGAGAAAUUUAA | 171 | SSSSS SSSSS SSSSS |
| | SfG * SfA * SmG * SfA * SmA * SfA * | AUC | | SSSS |
| | SmU * SfU * SfU * SfA * SfA * SfA | | | |
| | * SfU * SfC | | | |
| WV-21097 | fA * SfG * SfC * SfU * SfU * SfC * | AGCUUCGGAGAAAUUUA | 172 | SSSSS SOSSS SOO |
| | SmGfG * SfA * SmG * SfA * SmAmAfU * | AAUC | | SSSSS SS |
| | SfU * SfU * SfA * SfA * SfA * SfU | | | |
| | * SfC | | | |
| WV-21098 | fA * SfG * SfC * SfU * SfU * SfC * | AGCUUCGGAGAAAUUUA | 173 | SSSSS SOSSS SOO |
| | SmGfG * SfA * SmG * SfA * SmAmAfU * | AAU | | SSSSS S |
| | SfU * SfU * SfA * SfA * SfA * SfU | | | |
| WV-21099 | fG * SfC * SfU * SfU * SfC * SfG * | GCUUCGGAGAAAUUUAA | 174 | SSSSS SOSSS SOO |
| | SmGfA * SfG * SmA * SfA * SmAmUfU * | AUC | | SSSSS S |
| | SfU * SfA * SfA * SfA * SfU * SfC | | | |
| WV-21100 | fA * SfG * SfCn001fU * SfU * SfCn00 | AGCUUCGGAGAAAUUUA | 175 | SS nX SS nX SSSSS |
| | 1fG * SfG * SmA * SfG * SmA * SfA * | AAUC | | SSSSS nX SSS |
| | SmA * SfU * SfU * SfU * SfAn001fA | | | |
| | * SfA * SfU * SfC | | | |
| WV-21101 | fA * SfG * SfCn001fU * SfU * SfCn00 | AGCUUCGGAGAAAUUUA | 176 | SS nX SS nX SSSSS |
| | 1fG * SfG * SmA * SfG * SmA * SfA * | AAU | | SSSSS nX SS |
| | SmA * SfU * SfU * SfU * SfAn001fA | | | |
| | * SfA * SfU | | | |
| WV-21102 | fG * SfC * SfUn001fU * SfC * SfGn00 | GCUUCGGAGAAAUUUAA | 177 | SS nX SS nX SSSSS |
| | 1fG * SfA * SmG * SfA * SmA * SfA * | AUC | | SSSSS nX SS |
| | SmU * SfU * SfU * SfA * SfAn001fA | | | |
| | * SfU * SfC | | | |
| WV-21103 | fA * SfG * SfCn001fU * SfU * SfCn00 | AGCUUCGGAGAAAUUUA | 178 | SS nX SS nX OSSSS |
| | 1mGfG * SfA * SmG * SfA * SmAmAfU * | AAUC | | OOSSS nX SSS |
| | SfU * SfU * SfAn001fA * SfA * SfU | | | |
| | * SfC | | | |
| WV-21104 | fA * SfG * SfCn001fU * SfU * SfCn00 | AGCUUCGGAGAAAUUUA | 179 | SS nX SS nX OSSSS |
| | 1mGfG * SfA * SmG * SfA * SmAmAfU * | AAU | | OOSSS nX SS |
| | SfU * SfU * SfAn001fA * SfA * SfU | | | |
| WV-21105 | fG * SfC * SfUn001fU * SfC * SfGn00 | GCUUCGGAGAAAUUUAA | 180 | SS nX SS nX OSSSS |
| | 1mGfA * SfG * SmA * SfA * SmAmUfU * | AUC | | OOSSS nX SS |
| | SfU * SfA * SfAn001fA * SfU * SfC | | | |
| WV-24294 | fG * SfU * SfG * SfA * SfG * SfG * | GUGAGGAUUGCAGAAUU | 181 | SSSSS SSSSS SSSSS |
| | SfA * SfU * SmU * SfG * SmC * SfA * | UGU | | SSSS |
| | SmG * SfA * SfA * SfU * SfU * SfU | | | |
| | * SfG * SfU | | | |
| WV-24295 | fU * SfG * SfA * SfG * SfG * SfA * | UGAGGAUUGCAGAAUUU | 182 | SSSSS SSSSS SSSSS |
| | SfU * SfU * SmG * SfC * SmA * SfG * | GUU | | SSSS |
| | SmA * SfA * SfU * SfU * SfU * SfG | | | |
| | * SfU * SfU | | | |
| WV-24296 | fG * SfA * SfG * SfG * SfA * SfU * | GAGGAUUGCAGAAUUUG | 183 | SSSSS SSSSS SSSSS |
| | SfU * SfG * SmC * SfA * SmG * SfA * | UUC | | SSSS |
| | SmA * SfU * SfU * SfU * SfG * SfU | | | |
| | * SfU * SfC | | | |
| WV-24297 | fA * SfG * SfG * SfA * SfU * SfU * | AGGAUUGCAGAAUUUGU | 184 | SSSSS SSSSS SSSSS |
| | SfG * SfC * SmA * SfG * SmA * SfA * | UCA | | SSSS |
| | SmU * SfU * SfU * SfG * SfU * SfU | | | |
| | * SfC * SfA | | | |
| WV-24298 | fG * SfA * SfU * SfU * SfG * SfC * | GAUUGCAGAAUUUGUUC | 185 | SSSSS SSSSS SSSSS |
| | SfA * SfG * SmA * SfA * SmU * SfU * | ACU | | SSSS |
| | SmU * SfG * SfU * SfU * SfC * SfA | | | |
| | * SfC * SfU | | | |
| WV-24299 | fA * SfU * SfU * SfG * SfC * SfA * | AUUGCAGAAUUUGUUCA | 186 | SSSSS SSSSS SSSSS |
| | SfG * SfA * SmA * SfU * SmU * SfU * | CUG | | SSSS |
| | SmG * SfU * SfU * SfC * SfA * SfC | | | |
| | * SfU * SfG | | | |
| WV-24300 | fU * SfU * SfG * SfC * SfA * SfG * | UUGCAGAAUUUGUUCAC | 187 | SSSSS SSSSS SSSSS |
| | SfA * SfA * SmU * SfU * SmU * SfG * | UGA | | SSSS |
| | SmU * SfU * SfC * SfA * SfC * SfU | | | |
| | * SfG * SfA | | | |
| WV-24301 | fU * SfG * SfC * SfA * SfG * SfA * | UGCAGAAUUUGUUCACU | 188 | SSSSS SSSSS SSSSS |
| | SfA * SfU * SmU * SfU * SmG * SfU * | GAG | | SSSS |
| | SmU * SfC * SfA * SfC * SfU * SfG | | | |
| | * SfA * SfG | | | |
| WV-24302 | fU * SfU * SfC * SfU * SfU * SfA * | UUCUUACCUGGUUGACA | 189 | SSSSS SSSSS SSSSS |
| | SfC * SfC * SmU * SfG * SmG * SfU * | CUG | | SSSS |
| | SmU * SfG * SfA * SfC * SfA * SfC | | | |
| | * SfU * SfG | | | |
| WV-24303 | fU * SfC * SfU * SfU * SfA * SfC * | UCUUACCUGGUUGACAC | 190 | SSSSS SSSSS SSSSS |
| | SfC * SfU * SmG * SfG * SmU * SfU * | UGA | | SSSS |
| | SmG * SfA * SfC * SfA * SfC * SfU | | | |
| | * SfG * SfA | | | |
| WV-24304 | fC * SfU * SfU * SfA * SfC * SfC * | CUUACCUGGUUGACACU | 191 | SSSSS SSSSS SSSSS |
| | SfU * SfG * SmG * SfU * SmU * SfG * | GAU | | SSSS |
| | SmA * SfC * SfA * SfC * SfU * SfG | | | |
| | * SfA * SfU | | | |
| WV-24305 | fU * SfU * SfA * SfC * SfC * SfU * | UUACCUGGUUGACACUG | 192 | SSSSS SSSSS SSSSS |
| | SfG * SfG * SmU * SfU * SmG * SfA * | AUU | | SSSS |
| | SmC * SfA * SfC * SfU * SfG * SfA | | | |
| | * SfU * SfU | | | |
| WV-24306 | fA * SfC * SfC * SfU * SfG * SfG * | ACCUGGUUGACACUGAU | 193 | SSSSS SSSSS SSSSS |
| | SfU * SfU * SmG * SfA * SmC * SfA * | UAC | | SSSS |
| | SmC * SfU * SfG * SfA * SfU * SfU | | | |
| | * SfA * SfC | | | |
| WV-24307 | fC * SfC * SfU * SfG * SfG * SfU * | CCUGGUUGACACUGAUU | 194 | SSSSS SSSSS SSSSS |
| | SfU * SfG * SmA * SfC * SmA * SfC * | ACA | | SSSS |
| | SmU * SfG * SfA * SfU * SfU * SfA | | | |
| | * SfC * SfA | | | |
| WV-24308 | fC * SfU * SfG * SfG * SfU * SfU * | CUGGUUGACACUGAUUA | 195 | SSSSS SSSSS SSSSS |
| | SfG * SfA * SmC * SfA * SmC * SfU * | CAC | | SSSS |
| | SmG * SfA * SfU * SfU * SfA * SfC | | | |
| | * SfA * SfC | | | |
| WV-24309 | fU * SfG * SfG * SfU * SfU * SfG * | UGGUUGACACUGAUUAC | 196 | SSSSS SSSSS SSSSS |
| | SfA * SfC * SmA * SfC * SmU * SfG * | ACC | | SSSS |
| | SmA * SfU * SfU * SfA * SfC * SfA | | | |
| | * SfC * SfC | | | |
| WV-24356 | fG * SfU * SfG * SfA * SfG * SfG * | GUGAGGAUUGCAGAAUU | 197 | SSSSS SS SOSSS O |
| | SfA * SfU * SmUfG * SmC * SfA * SmG | UGU | | SSSSS S |
| | fA * SfA * SfU * SfU * SfU * SfG * | | | |
| | SfU | | | |
| WV-24357 | fU * SfG * SfA * SfG * SfG * SfA * | UGAGGAUUGCAGAAUUU | 198 | SSSSS SS SOSSS O |
| | SfU * SfU * SmGfC * SmA * SfG * SmA | GUU | | SSSSS S |
| | fA * SfU * SfU * SfU * SfG * SfU * | | | |
| | SfU | | | |
| WV-24358 | fG * SfA * SfG * SfG * SfA * SfU * | GAGGAUUGCAGAAUUUG | 199 | SSSSS SS SOSSS O |
| | SfU * SfG * SmCfA * SmG * SfA * SmA | UUC | | SSSSS S |
| | fU * SfU * SfU * SfG * SfU * SfU * | | | |
| | SfC | | | |
| WV-24359 | fA * SfG * SfG * SfA * SfU * SfU * | AGGAUUGCAGAAUUUGU | 200 | SSSSS SS SOSSS O |
| | SfG * SfC * SmAfG * SmA * SfA * SmU | UCA | | SSSSS S |
| | fU * SfU * SfG * SfU * SfU * SfC * | | | |
| | SfA | | | |
| WV-24360 | fG * SfG * SfA * SfU * SfU * SfG * | GGAUUGCAGAAUUUGUU | 201 | SSSSS SS SOSSS O |
| | SfC * SfA * SmGfA * SmA * SfU * SmU | CAC | | SSSSS S |
| | fU * SfG * SfU * SfU * SfC * SfA * | | | |
| | SfC | | | |
| WV-24361 | fG * SfA * SfU * SfU * SfG * SfC * | GAUUGCAGAAUUUGUUC | 202 | SSSSS SS SOSSS O |
| | SfA * SfG * SmAfA * SmU * SfU * SmU | ACU | | SSSSS S |
| | fG * SfU * SfU * SfC * SfA * SfC * | | | |
| | SfU | | | |
| WV-24362 | fA * SfU * SfU * SfG * SfC * SfA * | AUUGCAGAAUUUGUUCA | 203 | SSSSS |
| | SfG * SfA * SmAfU * SmU * SfU * SmG | CUG | | SS SOSSS O |
| | fU * SfU * SfC * SfA * SfC * SfU * | | | SSSSS S |
| | SfG | | | |
| WV-24363 | fU * SfU * SfG * SfC * SfA * SfG * | UUGCAGAAUUUGUUCAC | 204 | SSSSS SS SOSSS O |
| | SfA * SfA * SmUfU * SmU * SfG * SmU | UGA | | SSSSS S |
| | fU * SfC * SfA * SfC * SfU * SfG * | | | |
| | SfA | | | |
| WV-24364 | fU * SfG * SfC * SfA * SfG * SfA * | UGCAGAAUUUGUUCACU | 205 | SSSSS SS SOSSS O |
| | SfA * SfU * SmUfU * SmG * SfU * SmU | GAG | | SSSSS S |
| | fC * SfA * SfC * SfU * SfG * SfA * | | | |
| | SfG | | | |
| WV-24365 | fG * SfU * SfGn001fA * SfG * SfGn00 | GUGAGGAUUGCAGAAUU | 206 | SS nX SS nX S SOSSS |
| | 1fA * SfU * SmUfG * SmC * SfA * SmG | UGU | | OSSS nX SS |
| | fA * SfA * SfU * SfUn001fU * SfG * | | | |
| | SfU | | | |
| WV-24366 | fU * SfG * SfAn001fG * SfG * SfAn00 | UGAGGAUUGCAGAAUUU | 207 | SS nX SS nX S SOSSS |
| | 1fU * SfU * SmGfC * SmA * SfG * SmA | GUU | | OSSS nX SS |
| | fA * SfU * SfU * SfUn001fG * SfU * | | | |
| | SfU | | | |
| WV-24367 | fG * SfA * SfGn001fG * SfA * SfUn00 | GAGGAUUGCAGAAUUUG | 208 | SS nX SS nX S SOSSS |
| | 1fU * SfG * SmCfA * SmG * SfA * SmA | UUC | | OSSS nX SS |
| | fU * SfU * SfU * SfGn001fU * SfU * | | | |
| | SfC | | | |
| WV-24368 | fA * SfG * SfGn001fA * SfU * SfUn00 | AGGAUUGCAGAAUUUGU | 209 | SS nX SS nX S SOSSS |
| | 1fG * SfC * SmAfG * SmA * SfA * SmU | UCA | | OSSS nX SS |
| | fU * SfU * SfG * SfUn001fU * SfC * | | | |
| | SfA | | | |
| WV-24369 | fG * SfG * SfAn001fU * SfU * SfGn00 | GGAUUGCAGAAUUUGUU | 210 | SS nX SS nX S SOSSS |
| | 1fC * SfA * SmGfA * SmA * SfU * SmU | CAC | | OSSS nX SS |
| | fU * SfG * SfU * SfUn001fC * SfA * | | | |
| | SfC | | | |
| WV-24370 | fG * SfA * SfUn001fU * SfG * SfCn00 | GAUUGCAGAAUUUGUUC | 211 | SS nX SS nX S SOSSS |
| | 1fA * SfG * SmAfA * SmU * SfU * SmU | ACU | | OSSS nX SS |
| | fG * SfU * SfU * SfCn001fA * SfC * | | | |
| | SfU | | | |
| WV-24371 | fA * SfU * SfUn001fG * SfC * SfAn00 | AUUGCAGAAUUUGUUCA | 212 | SS nX SS nX S SOSSS |
| | 1fG * SfA * SmAfU * SmU * SfU * SmG | CUG | | OSSS nX SS |
| | fU * SfU * SfC * SfAn001fC * SfU * | | | |
| | SfG | | | |
| WV-24372 | fU * SfU * SfGn001fC * SfA * SfGn00 | UUGCAGAAUUUGUUCAC | 213 | SS nX SS nX S SOSSS |
| | 1fA * SfA * SmUfU * SmU * SfG * SmU | UGA | | OSSS nX SS |
| | fU * SfC * SfA * SfCn001fU * SfG * | | | |
| | SfA | | | |
| WV-24373 | fU * SfG * SfCn001fA * SfG * SfAn00 | UGCAGAAUUUGUUCACU | 214 | SS nX SS nX S SOSSS |
| | 1fA * SfU * SmUfU * SmG * SfU * SmU | GAG | | OSSS nX SS |
| | fC * SfA * SfC * SfUn001fG * SfA * | | | |
| | SfG | | | |
| WV-24374 | fG * SfU * SfGn001fA * SfG * SfGn00 | GUGAGGAUUGCAGAAUU | 215 | SS nX SS nX SSSSS |
| | 1fA * SfU * SmU * SfG * SmC * SfA * | UGU | | SSSSS nX SS |
| | SmG * SfA * SfA * SfU * SfUn001fU | | | |
| | * SfG * SfU | | | |
| WV-24375 | fU * SfG * SfAn001fG * SfG * SfAn00 | UGAGGAUUGCAGAAUUU | 216 | SS nX SS nX SSSSS |
| | 1fU * SfU * SmG * SfC * SmA * SfG * | GUU | | SSSSS nX SS |
| | SmA * SfA * SfU * SfU * SfUn001fG | | | |
| | * SfU * SfU | | | |
| WV-24376 | fG * SfA * SfGn001fG * SfA * SfUn00 | GAGGAUUGCAGAAUUUG | 217 | SS nX SS nX SSSSS |
| | 1fU * SfG * SmC * SfA * SmG * SfA * | UUC | | SSSSS nX SS |
| | SmA * SfU * SfU * SfU * SfGn001fU | | | |
| | * SfU * SfC | | | |
| WV-24377 | fA * SfG * SfGn001fA * SfU * SfUn00 | AGGAUUGCAGAAUUUGU | 218 | SS nX SS nX SSSSS |
| | 1fG * SfC * SmA * SfG * SmA * SfA * | UCA | | SSSSS nX SS |
| | SmU * SfU * SfU * SfG * SfUn001fU | | | |
| | * SfC * SfA | | | |
| WV-24378 | fG * SfG * SfAn001fU * SfU * SfGn00 | GGAUUGCAGAAUUUGUU | 219 | SS nX SS nX SSSSS |
| | 1fC * SfA * SmG * SfA * SmA * SfU * | CAC | | SSSSS nX SS |
| | SmU * SfU * SfG * SfU * SfUn001fC | | | |
| | * SfA * SfC | | | |
| WV-24379 | fG * SfA * SfUn001fU * SfG * SfCn00 | GAUUGCAGAAUUUGUUC | 220 | SS nX SS nX SSSSS |
| | 1fA * SfG * SmA * SfA * SmU * SfU * | ACU | | SSSSS nX SS |
| | SmU * SfG * SfU * SfU * SfCn001fA | | | |
| | * SfC * SfU | | | |
| WV-24380 | fA * SfU * SfUn001fG * SfC * SfAn00 | AUUGCAGAAUUUGUUCA | 221 | SS nX SS nX SSSSS |
| | 1fG * SfA * SmA * SfU * SmU * SfU * | CUG | | SSSSS nX SS |
| | SmG * SfU * SfU * SfC * SfAn001fC | | | |
| | * SfU * SfG | | | |
| WV-24381 | fU * SfU * SfGn001fC * SfA * SfGn00 | UUGCAGAAUUUGUUCAC | 222 | SS nX SS nX SSSSS |
| | 1fA * SfA * SmU * SfU * SmU * SfG * | UGA | | SSSSS nX SS |
| | SmU * SfU * SfC * SfA * SfCn001fU | | | |
| | * SfG * SfA | | | |
| WV-24382 | IfU * SfG * SfCn001fA * SfG * SfAn0 | UGCAGAAUUUGUUCACU | 223 | SS nX SS nX SSSSS |
| | 01fA * SfU * SmU * SfU * SmG * SfU | GAG | | SSSSS nX SS |
| | * SmU * SfC * SfA * SfC * SfUn001fG | | | |
| | * SfA * SfG | | | |
| WV-24383 | fC * SfC * SfC * SfA * SfU * SfA * | CCCAUAAAAGUUUUCUC | 224 | SSSSS SSSSS SSSSS |
| | SfA * SfA * SmA * SfG * SmU * SfU * | UGC | | SSSS |
| | SmU * SfU * SfC * SfU * SfC * SfU | | | |
| | * SfG * SfC | | | |
| WV-24384 | fA * SfC * SfC * SfC * SfA * SfU * | ACCCAUAAAAGUUUUCU | 225 | SSSSS SSSSS SSSSS |
| | SfA * SfA * SmA * SfA * SmG * SfU * | CUG | | SSSS |
| | SmU * SfU * SfU * SfC * SfU * SfC | | | |
| | * SfU * SfG | | | |
| WV-24385 | fA * SfA * SfC * SfC * SfC * SfA * | AACCCAUAAAAGUUUUC | 226 | SSSSS SSSSS SSSSS |
| | SfU * SfA * SmA * SfA * SmA * SfG * | UCU | | SSSS |
| | SmU * SfU * SfU * SfU * SfC * SfU | | | |
| | * SfC * SfU | | | |
| WV-24386 | fC * SfC * SfA * SfU * SfA * SfA * | CCAUAAAAGUUUUCUCU | 227 | SSSSS SS SOSSS O |
| | SfA * SfA * SmGfU * SmU SfU * SmUfC | GCA | | SSSSS S |
| | * SfU * SfC * SfU * SfG * SfC * Sf | | | |
| | A | | | |
| WV-24387 | fC * SfC * SfC * SfA * SfU * SfA * | CCCAUAAAAGUUUUCUC | 228 | SSSSS SS SOSSS O |
| | SfA * SfA * SmAfG * SmU * SfU * SmU | UGC | | SSSSS S |
| | fU * SfC * SfU * SfC * SfU * SfG * | | | |
| | SfC | | | |
| WV-24388 | fA * SfC * SfC * SfC * SfA * SfU * | ACCCAUAAAAGUUUUCU | 229 | SSSSS SS SOSSS O |
| | SfA * SfA * SmAfA * SmG * SfU * SmU | CUG | | SSSSS S |
| | fU * SfU * SfC * SfU * SfC * SfU * | | | |
| | SfG | | | |
| WV-24389 | fA * SfA * SfC * SfC * SfC * SfA * | AACCCAUAAAAGUUUUC | 230 | SSSSS SS SOSSS O |
| | SfU * SfA * SmAfA * SmA * SfG * SmU | UCU | | SSSSS S |
| | fU * SfU * SfU * SfC * SfU * SfC * | | | |
| | SfU | | | |
| WV-24390 | fC * SfC * SfAn001fU * SfA * SfAn00 | CCAUAAAAGUUUUCUCU | 231 | SS nX SS nX S SOSSS |
| | 1fA * SfA * SmGfU * SmU * SfU * SmU | GCA | | OSSS nX SS |
| | fC * SfU * SfC * SfUn001fG * SfC * | | | |
| | SfA | | | |
| WV-24391 | fC * SfC * SfCn001fA * SfU * SfAn00 | CCCAUAAAAGUUUUCUC | 232 | SS nX SS nX S SOSSS |
| | 1fA * SfA * SmAfG * SmU * SfU * SmU | UGC | | OSSS nX SS |
| | fU * SfC SfU * SfCn001fU * SfG * Sf | | | |
| | C | | | |
| WV-24392 | fA * SfC * SfCn001fC * SfA * SfUn00 | SmGACCCAUAAAAGUUUUCU | 233 | SS nX SS nX S SOSSS |
| | 1fA * SfA * SmAfA * * SfU * SmUfU * | CUG | | OSSS nX SS |
| | SfU * SfC * SfUn001fC * SfU * SfG | | | |
| WV-24393 | fA * SfA * SfCn001fC * SfC * SfAn00 | AACCCAUAAAAGUUUUC | 234 | SS nX SS nX S SOSSS |
| | 1fU * SfA * SmAfA * SmA SfG * SmUfU | UCU | | OSSS nX SS |
| | * SfU * SfU * SfCn001fU * SfC * Sf | | | |
| | U | | | |
| WV-24394 | fC * SfC * SfAn001fU* SfA * SfAn001 | CCAUAAAAGUUUUCUCU | 235 | SS nX SS nX SSSSS |
| | fA * SfA * SmG * SfU * SmU * SfU * | GCA | | SSSSS nX SS |
| | SmU * SfC * SfU * SfC * SfUn001fG * | | | |
| | SfC * SfA | | | |
| WV-24395 | fC * SfC * SfCn001fA * SfU * SfAn00 | CCCAUAAAAGUUUUCUC | 236 | SS nX SS nX SSSSS |
| | 1fA * SfA * SmA * SfG * SmU * SfU * | UGC | | SSSSS nX SS |
| | SmU * SfU * SfC * SfU * SfCn001fU | | | |
| | * SfG * SfC | | | |
| WV-24396 | fA * SfC * SfCn001fC * SfA * SfUn00 | ACCCAUAAAAGUUUUCU | 237 | SS nX SS nX SSSSS |
| | 1fA * SfA * SmA * SfA * SmG * SfU * | CUG | | SSSSS nX SS |
| | SmU * SfU * SfU * SfC * SfUn001fC | | | |
| | * SfU * SfG | | | |
| WV-24397 | fA * SfA * SfCn001fC * SfC * SfAn00 | AACCCAUAAAAGUUUUC | 238 | SS nX SS nX SSSSS |
| | 1fU * SfA * SmA * SfA * SmA * SfG * | UCU | | SSSSS nX SS |
| | SmU * SfU * SfU * SfU * SfCn001fU | | | |
| | * SfC * SfU | | | |
| WV-27911 | fG fG fAn001 fU fU fGn001 fC fA mG | GGAUUGCAGAAUUUGUU | 239 | SSnXSS nXSSOS |
| | fA mA fU mU fU fG fU fU fC fA fC | CAC | | SSOSS SSSS |
| WV-27912 | fG fG fAn001 fU fU fG fC fA mG fA m | GGAUUGCAGAAUUUGUU | 240 | SSnXSS SSSOS |
| | A fU mU fU fG fU fU fC fA fC | CAC | | SSOSS SSSS |
| WV-27913 | fG fG fA fU fU fGn001 fC fA mG fA m | GGAUUGCAGAAUUUGUU | 241 | SSSSS nXSSOS |
| | A fU mU fU fG fU fU fC fA fC | CAC | | SSOSS SSSS |
| WV-27914 | fG fG fA fU fU fGn001 fC fA mG fA m | GGAUUGCAGAAUUUGUU | 242 | SSOSS nXSSOS |
| | A fU mU fU fG fU fU fC fA fC | CAC | | SSSSS SSSS |
| WV-27915 | fG fG fAn001 fU fU fGn001 fC fA mG | GGAUUGCAGAAUUUGUU | 243 | SSnXSS nXSSOS |
| | fA mA fU mU fU fG fU fU fC fA fC | CAC | | SSSSS SSSS |
| WV-27916 | fG fG fA fU fU fG fC fA mG fA mA fU | GGAUUGCAGAAUUUGUU | 244 | SSSSS SSSOS SSOSS |
| | mU fU fG fU fUn001 fCn001 fAn001 f | CAC | | SnXnXnX |
| | C | | | |
| WV-27917 | fGn001 fGn001 fAn001 fU fU fG fC fA | GGAUUGCAGAAUUUGUU | 245 | nXnXnXSS SSSOS |
| | mG fA mA fU mU fU fG fU fU fC fA f | CAC | | SSSSS SSSS |
| | C | | | |
| WV-27918 | fGn001 fGn001 fA fU fU fG fC fA mG | GGAUUGCAGAAUUUGUU | 246 | nXnXSSS SSSOS |
| | fA mA fU mU fU fG fU fU fC fA fC | CAC | | SSSSS SSSS |
| WV-28105 | fG fG fA fU fU fG fC fA mG fA mA fU | GGAUUGCAGAAUUUGUU | 247 | XXXXX XXXXX |
| | mU fU fG fU fU fC fA fC | CAC | | XXXXX XXXX |
| WV-28106 | mG mG mA mU mU mG mC mA mG mA mA mU | GGAUUGCAGAAUUUGUU | 248 | XX XXX XXXXX |
| | mU mU mG mU mU mC mA mC | CAC | | XXXXX XXXX |
| WV-28107 | Geo Geo Aeo Teo Teo Geo m5Ceo Aeo G | GGATTGCAGAATTTGTTC | 249 | XXXXX XXXXX |
| | eo Aeo Aeo Teo Teo Teo Geo Teo Teo | AC | | XXXXX XXXX |
| | m5Ceo Aeo m5Ceo | | | |
| WV-28108 | fG fG fA fU fU fG fC fA mG fA mA fU | GGAUUGCAGAAUUUGUU | 250 | SSSSS SOSSS SSSSS |
| | mU fU fG fU fU fC fA fC | CAC | | SSSS |
| WV-28109 | fG fG fA fU fU fG fC fA mG fA mA fU | GGAUUGCAGAAUUUGUU | 251 | SSSSS SSOSS SSSSS |
| | mU fU fG fU fU fC fA fC | CAC | | SSSS |
| WV-28110 | fG fG fA fU fU fG fC fA mG fA mA fU | GGAUUGCAGAAUUUGUU | 252 | SSSSS SSSOS SSSSS |
| | mU fU fG fU fU fC fA fC | CAC | | SSSS |
| WV-28111 | fG fG fA fU fU fG fC fA mG fA mA fU | GGAUUGCAGAAUUUGUU | 253 | SSSSS SSSSO SSSSS |
| | mU fU fG fU fU fC fA fC | CAC | | SSSS |
| WV-28112 | fG fG fA fU fU fG fC fA mG fA mA fU | GGAUUGCAGAAUUUGUU | 254 | SSSSS SSSSS OSSSS |
| | mU fU fG fU fU fC fA fC | CAC | | SSSS |
| WV-28113 | fG fG fA fU fU fG fC fA mG fA mA fU | GGAUUGCAGAAUUUGUU | 255 | SSSSS SSSSS SOSSS |
| | mU fU fG fU fU fC fA fC | CAC | | SSSS |
| WV-28114 | fG fG fA fU fU fG fC fA mG fA mA fU | GGAUUGCAGAAUUUGUU | 256 | SSSSS SSSSS SSOSS |
| | mU fU fG fU fU fC fA fC | CAC | | SSSS |
| WV-28115 | fG fG fA fU fU fG fC fA mG fA mA fU | GGAUUGCAGAAUUUGUU | 257 | SSSSS SSSSS SSSOS |
| | mU fU fG fU fU fC fA fC | CAC | | SSSS |
| WV-28116 | fG fG fA fU fU fG mC fA mG fA mA fU | GGAUUGCAGAAUUUGUU | 258 | SSSSS SOSOS |
| | mU fU fG fU fU fC fA fC | CAC | | OSOSS SSSS |
| WV-28117 | fG fG fA fU fU fG mC fA mG mA fA mU | GGAUUGCAGAAUUUGUU | 259 | SSSSS SOSOS |
| | mU fU fG fU fU fC fA fC | CAC | | SOOSS SSSS |
| WV-28118 | fG fG fA fU fU fG mC fA fG mA fA mU | GGAUUGCAGAAUUUGUU | 260 | SSSSS SOSSS SOSSS |
| | mU fU fG fU fU fC fA fC | CAC | | SSSS |
| WV-28119 | fG fA fU fU fG fC fA fG mA fA mU fU | GAUUGCAGAAUUUGUUC | 261 | XXXXX XXXXX |
| | mU fG fU fU fC fA fC fU | ACU | | XXXXX XXXX |
| WV-28120 | mG mA mU mU mG mC mA mG mA mA mU mU | GAUUGCAGAAUUUGUUC | 262 | XXXXX XXXXX XXXXX |
| | mU mG mU mU mC mA mC mU | ACU | | XXXX |
| WV-28121 | Geo Aeo Teo Teo Geo m5Ceo Aeo Geo A | GATTGCAGAATTTGTTCA | 263 | XXXXX XXXXX |
| | eo Aeo Teo Teo Teo Geo Teo Teo m5Ce | CT | | XXXXX XXXX |
| | o Aeo m5Ceo Teo | | | |
| WV-28122 | fG fA fU fU fG fC fA fG mA fA mU fU | GAUUGCAGAAUUUGUUC | 264 | SSSSS SOSSS SSSSS |
| | mU fG fU fU fC fA fC fU | ACU | | SSSS |
| WV-28123 | fG fA fU fU fG fC fA fG mA fA mU fU | GAUUGCAGAAUUUGUUC | 265 | SSSSS SSOSS SSSSS |
| | mU fG fU fU fC fA fC fU | ACU | | SSSS |
| WV-28124 | fG fA fU fU fG fC fA fG mA fA mU fU | GAUUGCAGAAUUUGUUC | 26 | SSSSS SSSOS SSSSS |
| | mU fG fU fU fC fA fC fU | ACU | | SSSS |
| WV-28125 | fG fA fU fU fG fC fA fG mA fA mU fU | GAUUGCAGAAUUUGUUC | 267 | SSSSS SSSSO SSSSS |
| | mU fG fU fU fC fA fC fU | ACU | | SSSS |
| WV-28126 | fG fA fU fU fG fC fA fG mA fA mU fU | GAUUGCAGAAUUUGUUC | 268 | SSSSS SSSSS OSSSS |
| | mU fG fU fU fC fA fC fU | ACU | | SSSS |
| WV-28127 | fG fA fU fU fG fC fA fG mA fA mU fU | GAUUGCAGAAUUUGUUC | 269 | SSSSS SSSSS SOSSS |
| | mU fG fU fU fC fA fC fU | ACU | | SSSS |
| WV-28128 | fG fA fU fU fG fC fA fG mA fA mU fU | GAUUGCAGAAUUUGUUC | 270 | SSSSS SSSSS SSOSS |
| | mU fG fU fU fC fA fC fU | ACU | | SSSS |
| WV-28129 | fG fA fU fU fG fC fA fG mA fA mU fU | GAUUGCAGAAUUUGUUC | 271 | SSSSS SSSSS SSSOS |
| | mU fG fU fU fC fA fC fU | ACU | | SSSS |
| WV-28130 | fG fA fU fU fG fC mA fG mA fA mU fU | GAUUGCAGAAUUUGUUC | 272 | SSSSS SOSOS |
| | mU fG fU fU fC fA fC fU | ACU | | OSOSS SSSS |
| WV-28131 | fG fA fU fU fG fC mA fG mA mA fU mU | GAUUGCAGAAUUUGUUC | 273 | SSSSS SOSOS |
| | mU fG fU fU fC fA fC fU | ACU | | SOOSS SSSS |
| WV-28132 | fG fA fU fU fG fC mA fG fA mA fU mU | GAUUGCAGAAUUUGUUC | 274 | SSSSS SOSSS SOSSS |
| | mU fG fU fU fC fA fC fU | ACU | | SSSS |
| WV-28133 | fU fG fCn001 fA fG fAn001 fA fU mU | UGCAGAAUUUGUUCACU | 275 | XXnXXX nXXXXX |
| | fU mG fU mU fC fA fC fUn001 fG fA f | GAG | | XXXXX XnXXX |
| | G | | | |
| WV-28134 | mU mG mCn001 mA mG mAn001 mA mU mU | UGCAGAAUUUGUUCACU | 276 | XXnXXX nXXXXX |
| | mU mG mU mU mC mA mC mUn001 mG mA m | GAG | | XXXXX XnXXX |
| | G | | | |
| WV-28135 | Teo Geo m5Ceo n001Aeo Geo Aeo n001A | TGCAGAATTTGTTCACTG | 277 | XXnXXX nXXXXX |
| | eo Teo Teo Teo Geo Teo Teo m5Ceo Ae | AG | | XXXXX XnXXX |
| | o m5Ceo Teo n001Geo Aeo Geo | | | |
| WV-28136 | fU fG fCn001 fA fG fAn001 fA fU mU | UGCAGAAUUUGUUCACU | 278 | SSnXSS nXOSSS |
| | fU mG fU mU fC fA fC fUn001 fG fA f | GAG | | SSSSS SnXSS |
| | G | | | |
| WV-28137 | fU fG fCn001 fA fG fAn001 fA fU mU | UGCAGAAUUUGUUCACU | 279 | SSnXSS nXSOSS |
| | fU mG fU mU fC fA fC fUn001 fG fA f | GAG | | SSSSS SnXSS |
| | G | | | |
| WV-28138 | fU fG fCn001 fA fG fAn001 fA fU mU | UGCAGAAUUUGUUCACU | 280 | SSnXSS nXSSOS |
| | fU mG fU mU fC fA fC fUn001 fG fA f | GAG | | SSSSS SnXSS |
| | G | | | |
| WV-28139 | fU fG fCn001 fA fG fAn001 fA fU mU | UGCAGAAUUUGUUCACU | 281 | SSnXSS nXSSSO |
| | fU mG fU mU fC fA fC fUn001 fG fA f | GAG | | SSSSS SnXSS |
| | G | | | |
| WV-28140 | fU fG fCn001 fA fG fAn001 fA fU mU | UGCAGAAUUUGUUCACU | 282 | SSnXSS nXSSSS |
| | fU mG fU mU fC fA fC fUn001 fG fA f | GAG | | OSSSS SnXSS |
| | G | | | |
| WV-28141 | fU fG fCn001 fA fG fAn001 fA fU mU | UGCAGAAUUUGUUCACU | 283 | SSnXSS nXSSSS |
| | fU mG fU mU fC fA fC fUn001 fG fA f | GAG | | SOSSS SnXSS |
| | G | | | |
| WV-28142 | fU fG fCn001 fA fG fAn001 fA fU mU | UGCAGAAUUUGUUCACU | 284 | SSnXSS nXSSSS |
| | fU mG fU mU fC fA fC fUn001 fG fA f | GAG | | SSOSS SnXSS |
| | G | | | |
| WV-28143 | fU fG fCn001 fA fG fAn001 fA fU mU | UGCAGAAUUUGUUCACU | 285 | SSnXSS nXSSSS |
| | fU mG fU mU fC fA fC fUn001 fG fA f | GAG | | SSSOS SnXSS |
| | G | | | |
| WV-28144 | fU fG fCn001 fA fG fAn001 mA fU mU | UGCAGAAUUUGUUCACU | 286 | SSnXSS nXOSOS |
| | fU mG fU mU fC fA fC fUn001 fG fA f | GAG | | OSOSS SnXSS |
| | G | | | |
| WV-28145 | fU fG fCn001 fA fG fAn001 mA fU mU | UGCAGAAUUUGUUCACU | 287 | SSnXSS nXOSOS |
| | mU fG mU mU fC fA fC fUn001 fG fA f | GAG | | SOOSS SnXSS |
| | G | | | |
| WV-28146 | fU fG fCn001 fA fG fAn001 mA fU fU | UGCAGAAUUUGUUCACU | 288 | SSnXSS nXOSSS |
| | mU fG mU mU fC fA fC fUn001 fG fA f | GAG | | SOSSS SnXSS |
| | G | | | |
| WV-30205 | fU fG fCn001R fA fG fAn001R fA fU m | UGCAGAAUUUGUUCACU | 289 | SSnRSS nRSSSS |
| | U fU mG fU mU fC fA fC fUn001R fG f | GAG | | SSSSS SnRSS |
| | A fG | | | |
| WV-32013 | fG fA fU fU fG fC fA mG fA mA fU mU | GAUUGCAGAAUUUGUUC | 290 | SSSSS SSSSS SSSSS |
| | fU fG fU fU fC fA fC | AC | | SSS |
| WV-32014 | fA fU fU fG fC fA mG fA mA fU mU fU | AUUGCAGAAUUUGUUCA | 291 | SSSSS SSSSS SSSSS |
| | fG fU fU fC fA fC | C | | SS |
| WV-32015 | fU fU fG fC fA mG fA mA fU mU fU fG | UUGCAGAAUUUGUUCAC | 292 | SSSSS SSSSS SSSSS |
| | fU fU fC fA fC | | | S |
| WV-32016 | fG fG fA fU fU fG fC fA mG fA mA fU | GGAUUGCAGAAUUUGUU | 293 | SSSSS SSSSS SSSSS |
| | mU fU fG fU fU fC fA | CA | | SSS |
| WV-32017 | fG fG fA fU fU fG fC fA mG fA mA fU | GGAUUGCAGAAUUUGUU | 294 | SSSSS SSSSS SSSSS |
| | mU fU fG fU fU fC | C | | SS |
| WV-32018 | fG fG fA fU fU fG fC fA mG fA mA fU | GGAUUGCAGAAUUUGUU | 295 | SSSSS SSSSS SSSSS |
| | mU fU fG fU fU | | | S |
| WV-32019 | fC fC fA fA fG fG fA fA mU fC mA fC | CCAAGGAAUCACACUCA | 296 | SSSSS SSSSS SSSSS |
| | mA fC fU fC fA fC fA fC | CAC | | SSSS |
| WV-32020 | fC fA fA fG fG fA fA fU mC fA mC fA | CAAGGAAUCACACUCAC | 297 | SSSSS SSSSS SSSSS |
| | mC fU fC fA fC fA fC fA | ACA | | SSSS |
| WV-32021 | fA fA fG fG fA fA fU fC mA fC mA fC | AAGGAAUCACACUCACA | 298 | SSSSS SSSSS SSSSS |
| | mU fC fA fC fA fC fA fU | CAU | | SSSS |
| WV-32022 | fA fG fG fA fA fU fC fA mC fA mC fU | AGGAAUCACACUCACAC | 299 | SSSSS SSSSS SSSSS |
| | mC fA fC fA fC fA fU fC | AUC | | SSSS |
| WV-32023 | fG fA fA fU fC fA fC fA mC fU mC fA | GAAUCACACUCACACAU | 300 | SSSSS SSSSS SSSSS |
| | mC fA fC fA fU fC fU fG | CUG | | SSSS |
| WV-32024 | fA fA fU fC fA fC fA fC mU fC mA fC | AAUCACACUCACACAUC | 301 | SSSSS SSSSS SSSSS |
| | mA fC fA fU fC fU fG fG | UGG | | SSSS |
| WV-32025 | fA fU fC fA fC fA fC fU mC fA mC fA | AUCACACUCACACAUCU | 302 | SSSSS SSSSS SSSSS |
| | mC fA fU fC fU fG fG fC | GGC | | SSSS |
| WV-32026 | fU fC fA fC fA fC fU fC mA fC mA fC | UCACACUCACACAUCUG | 303 | SSSSS SSSSS SSSSS |
| | mA fU fC fU fG fG fC fA | GCA | | SSSS |
| WV-32027 | fC fC fA fA fG fG fA fA mU fC mA fC | CCAAGGAAUCACACUCA | 304 | SSSSS SSSOS SSOSS |
| | mA fC fU fC fA fC fA fC | CAC | | SSSS |
| WV-32028 | fC fA fA fG fG fA fA fU mC fA mC fA | CAAGGAAUCACACUCAC | 305 | SSSSS SSSOS SSOSS |
| | mC fU fC fA fC fA fC fA | ACA | | SSSS |
| WV-32029 | fA fA fG fG fA fA fU fC mA fC mA fC | AAGGAAUCACACUCACA | 306 | SSSSS SSSOS SSOSS |
| | mU fC fA fC fA fC fA fU | CAU | | SSSS |
| WV-32030 | fA fG fG fA fA fU fC fA mC fA mC fU | AGGAAUCACACUCACAC | 307 | SSSSS SSSOS SSOSS |
| | mC fA fC fA fC fA fU fC | AUC | | SSSS |
| WV-32031 | fG fG fA fA fU fC fA fC mA fC mU fC | GGAAUCACACUCACACA | 308 | SSSSS SSSOS SSOSS |
| | mA fC fA fC fA fU fC fU | UCU | | SSSS |
| WV-32032 | fG fA fA fU fC fA fC fA mC fU mC fA | GAAUCACACUCACACAU | 309 | SSSSSs SSSOS SSOSS |
| | mC fA fC fA fU fC fU fG | CUG | | SSSS |
| WV-32033 | fA fA fU fC fA fC fA fC mU fC mA fC | AAUCACACUCACACAUC | 310 | SSSSS SSSOS SSOSS |
| | mA fC fA fU fC fU fG fG | UGG | | SSSS |
| WV-32034 | fA fU fC fA fC fA fC fU mC fA mC fA | AUCACACUCACACAUCU | 311 | SSSSS SSSOS SSOSS |
| | mC fA fU fC fU fG fG fC | GGC | | SSSS |
| WV-32035 | fU fC fA fC fA fC fU fC mA fC mA fC | UCACACUCACACAUCUG | 312 | SSSSS SSSOS SSOSS |
| | mA fU fC fU fG fG fC fA | GCA | | SSSS |
| WV-32036 | fC fC fAn001 fA fG fGn001 fA fA mU | CCAAGGAAUCACACUCA | 313 | SSnXSS nXSSOS |
| | fC mA fC mA fC fU fC fAn001 fC fA f | CAC | | SSOSS SnXSS |
| | C | | | |
| WV-32037 | fC fA fAn001 fG fG fAn001 fA fU mC | CAAGGAAUCACACUCAC | 314 | SSnXSS nXSSOS |
| | fA mC fA mC fU fC fA fCn001 fA fC f | ACA | | SSOSS SnXSS |
| | A | | | |
| WV-32038 | fA fA fGn001 fG fA fAn001 fU fC mA | AAGGAAUCACACUCACA | 315 | SSnXSS nXSSOS |
| | fC mA fC mU fC fA fC fAn001 fC fA f | CAU | | SSOSS SnXSS |
| | U | | | |
| WV-32039 | fA fG fGn001 fA fA fUn001 fC fA mC | AGGAAUCACACUCACAC | 316 | SSnXSS nXSSOS |
| | fA mC fU mC fA fC fA fCn001 fA fU f | AUC | | SSOSS SnXSS |
| | C | | | |
| WV-32040 | fG fG fAn001 fA fU fCn001 fA fC mA | GGAAUCACACUCACACA | 317 | SSnXSS nXSSOS |
| | fC mU fC mA fC fA fC fAn001 fU fC f | UCU | | SSOSS SnXSS |
| | U | | | |
| WV-32041 | fG fA fAn001 fU fC fAn001 fC fA mC | GAAUCACACUCACACAU | 318 | SSnXSS nXSSOS |
| | fU mC fA mC fA fC fA fUn001 fC fU f | CUG | | SSOSS SnXSS |
| | G | | | |
| WV-32042 | fA fA fUn001 fC fA fCn001 fA fC mU | AAUCACACUCACACAUC | 319 | SSnXSS nXSSOS |
| | fC mA fC mA fC fA fU fCn001 fU fG f | UGG | | SSOSS SnXSS |
| | G | | | |
| WV-32043 | fA fU fCn001 fA fC fAn001 fC fU mC | AUCACACUCACACAUCU | 320 | SSnXSS nXSSOS |
| | fA mC fA mC fA fU fC fUn001 fG fG f | GGC | | SSOSS SnXSS |
| | C | | | |
| WV-32044 | fU fC fAn001 fC fA fCn001 fU fC mA | UCACACUCACACAUCUG | 321 | SSnXSS nXSSOS |
| | fC mA fC mA fU fC fU fGn001 fG fC f | GCA | | SSOSS SnXSS |
| | A | | | |
| WV-32045 | fC fC fAn001 fA fG fGn001 fA fA mU | CCAAGGAAUCACACUCA | 322 | SSnXSS nXSSSS |
| | fC mA fC mA fC fU fC fAn001 fC fA f | CAC | | SSSSS SnXSS |
| | C | | | |
| WV-32046 | fC fA fAn001 fG fG fAn001 fA fU mC | CAAGGAAUCACACUCAC | 323 | SSnXSS nXSSSS |
| | fA mC fA mC fU fC fA fCn001 fA fC f | ACA | | SSSSS SnXSS |
| | A | | | |
| WV-32047 | fA fA fGn001 fG fA fAn001 fU fC mA | AAGGAAUCACACUCACA | 32 | SSnXSS nXSSSS |
| | fC mA fC mU fC fA fC fAn001 fC fA f | CAU | | SSSSS SnXSS |
| | U | | | |
| WV-32048 | fA fG fGn001 fA fA fUn001 fC fA mC | AGGAAUCACACUCACAC | 325 | SSnXSS nXSSSS |
| | fA mC fU mC fA fC fA fCn001 fA fU f | AUC | | SSSSS SnXSS |
| | C | | | |
| WV-32049 | fG fG fAn001 fA fU fCn001 fA fC mA | GGAAUCACACUCACACA | 326 | SSnXSS nXSSSS |
| | fC mU fC mA fC fA fC fAn001 fU fC f | UCU | | SSSSS SnXSS |
| | U | | | |
| WV-32050 | fG fA fAn001 fU fC fAn001 fC fA mC | GAAUCACACUCACACAU | 327 | SSnXSS nXSSSS |
| | fU mC fA mC fA fC fA fUn001 fC fU f | CUG | | SSSSS SnXSS |
| | G | | | |
| WV-32051 | fA fA fUn001 fC fA fCn001 fA fC mU | AAUCACACUCACACAUC | 328 | SSnXSS nXSSSS |
| | fC mA fC mA fC fA fU fCn001 fU fG f | UGG | | SSSSS SnXSS |
| | G | | | |
| WV-32052 | fA fU fCn001 fA fC fAn001 fC fU mC | AUCACACUCACACAUCU | 329 | SSnXSS nXSSSS |
| | fA mC fA mC fA fU fC fUn001 fG fG f | GGC | | SSSSS SnXSS |
| | C | | | |
| WV-32053 | fU fC fAn001 fC fA fCn001 fU fC mA | UCACACUCACACAUCUG | 330 | SSnXSS nXSSSS |
| | fC mA fC mA fU fC fU fGn001 fG fC f | GCA | | SSSSS SnXSS |
| | A | | | |
| WV-32052 | fA*SfU*SfCn001fA*SfC*SfAn001fC*SfU* | AUCACACUCACACAUCU | 331 | SSnXSSnXSSSSSSSS |
| | SmC*SfA*SmC* SfA*SmC*SfA*SfU*SfC*Sf | GGC | | SSnXSS |
| | Un001fG*SfG*SfC | | | |
| WV-32053 | fU*SfC*SfAn001fC*SfA*SfCn001fU*SfC* | UCACACUCACACAUCUG | 332 | SSnXSSnXSSSSSSSS |
| | SmA*SfC*SmA* SfC*SmA*SfU*SfC*SfU*Sf | GCA | | SSnXSS |
| | Gn001fG*SfC*SfA | | | |
| WV-33863 | fU*SfG*SfCn001RfA*SfG*SfAn001RfA*Sf | UGCAGAAUUUGUUCACU | 333 | SSnRSSnRSSOSSSO |
| | U*SmUfU*SmG *SfU*SmUfC*SfA*SfC*SfUn | GAG | | SSSnRSS |
| | 001RfG*SfA*SfG | | | |
| WV-36437 | fUn001RfG*SfC*SfA*SfG*SfAn001RfA*Sf | UGCAGAAUUUGUUCACU | 334 | nRSSSSnRSSOSSSO |
| | U*SmUfU*SmG *SfU*SmUfC*SfA*SfC*SfUn | GAG | | SSSnRSS |
| | 001RfG*SfA*SfG | | | |
| WV-36439 | fUn001RfG*SfC*SfA*SfG*SfAn001RfA*Sf | UGCAGAAUUUGUUCACU | 335 | nRSSSSnRSSOSSSO |
| | U*SmUfU*SmG *SfU*SmUfC*SfA*SfC*SfU* | GAG | | SSSSSnR |
| | SfG*SfAn001RfG | | | |
| WV-36864 | fG*SfCn001RfA*SfG*SfAn001RfA*SfU*Sm | GCAGAAUUUGUUCACUG | 336 | SnRSSnRSSSSSSSSS |
| | U*SfU*SmG*S fU*SmU*SfC*SfA*SfC*SfUn | AG | | SnRSS |
| | 001RfG*SfA*SfG | | | |
| WV-36865 | fCn001RfA*SfG*SfAn001RfA*SfU*SmU*Sf | CAGAAUUUGUUCACUGA | 337 | nRSSnRSSSSSSSSSS |
| | U*SmG*SfU*S mU*SfC*SfA*SfC*SfUn001R | G | | nRSS |
| | fG*SfA*SfG | | | |
| WV-36866 | fU*SfG*SfCn001RfA*SfG*SfAn001RfA*Sf | UGCAGAAUUUGUUCACU | 338 | SSnRSSnRSSSSSSSS |
| | U*SmU*SfU*S mG*SfU*SmU*SfC*SfA*SfC* | GA | | SSnRS |
| | SfUn001RfG*SfA | | | |
| WV-36867 | fU*SfG*SfCn001RfA*SfG*SfAn001RfA*Sf | UGCAGAAUUUGUUCACU | 339 | SSnRSSnRSSSSSSSS |
| | U*SmU*SfU*S mG*SfU*SmU*SfC*SfA*SfC* | G | | SSnR |
| | SfUn001RfG | | | |
| WV-36868 | fG*SfCn001RfA*SfG*SfAn001RfA*SfU*Sm | GCAGAAUUUGUUCACUG | 340 | SnRSSnRSSSSSSSSS |
| | U*SfU*SmG*S fU*SmU*SfC*SfA*SfC*SfUn | A | | SnRS |
| | 001RfG*SfA | | | |

### Notes:

Spaces in Table A1 are utilized for formatting and readability, e.g., SS nX SS nX S SOSSS OSSS nX SS illustrates the same stereochemistry as SSnXSSnXSSOSSSOSSSnXSS; * S and *S both indicate a phosphorothioate internucleotidic linkage wherein the linkage phosphorus has Sp configuration (S); etc. Description, Base Sequence and Stereochemistry/Linkage, due to their length, may be divided into multiple lines in Table A1. Unless otherwise specified, all oligonucleotides in Table A1 are single-stranded. As appreciated by those skilled in the art, nucleoside units are unmodified and contain unmodified nucleobases and 2′-deoxy sugars unless otherwise indicated with modifications (e.g., modified with r, m, m5, eo, etc.); linkages, unless otherwise indicated, are natural phosphate linkages; and acidic/basic groups may independently exist in their salt forms. Moieties and modifications in oligonucleotides (or other compounds, e.g., those useful for preparing provided oligonucleotides comprising these moieties or modifications):
f: 2′-F;
m: 2′-OMe;
m5 (or m5C): methyl at 5-position of C (nucleobase is 5-methylcytosine);
m5Ceo: 5-methyl 2′-O-methoxyethyl C;
eo: 2′-MOE (2′-OCH₂CH₂OCH₃);
O, PO: phosphodiester (phosphate), which can be an internucleotidic linkage (a natural phosphate linkage). Phosphodiesters are typically indicated with “O” in the Stereochemistry/Linkage column and are typically not marked in the Description column; if no linkage is indicated in the Description column, it is typically a phosphodiester unless otherwise indicated;
*, PS: phosphorothioate, which can be an internucleotidic linkage (a phosphorothioate internucleotidic linkage). * (as opposed to * R or * S) indicates a phosphorothioate which is not chirally controlled;
R, Rp: Phosphorothioate in the Rp configuration. Note that * R in Description indicates a single phosphorothioate linkage in the Rp configuration;
S, Sp: Phosphorothioate in the Sp configuration. Note that * S in Description indicates a single phosphorothioate linkage in the Sp configuration;
X: stereorandom phosphorothioate;
n001:

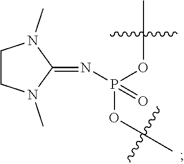 [SMILES: *OP(=O)(N=C1N(C)CCN1C)O*]

nX: stereorandom n001; and
nR: n001R: n001 in the Rp configuration.

### Lengths

As appreciated by those skilled in the art, oligonucleotides can be of various lengths to provide desired properties and/or activities for various uses. Many technologies for assessing, selecting and/or optimizing oligonucleotide length are available in the art and can be utilized in accordance with the present disclosure. As demonstrated herein, in many embodiments, provided oligonucleotides are of suitable lengths to hybridize with their targets and reduce levels of their targets and/or an encoded product thereof. In some embodiments, an oligonucleotide is long enough to recognize a target nucleic acid (e.g., an USH2A mRNA). In some embodiments, an oligonucleotide is sufficiently long to distinguish between a target nucleic acid and other nucleic acids (e.g., a nucleic acid having a base sequence which is not USH2A) to reduce off-target effects. In some embodiments, an USH2A oligonucleotide is sufficiently short to reduce complexity of manufacture or production and to reduce cost of products.

In some embodiments, the base sequence of an oligonucleotide is about 10-500 nucleobases in length. In some embodiments, a base sequence is about 10-500 nucleobases in length. In some embodiments, a base sequence is about 10-50 nucleobases in length. In some embodiments, a base sequence is about 15-50 nucleobases in length. In some embodiments, a base sequence is from about 15 to about 30 nucleobases in length. In some embodiments, a base sequence is from about 10 to about 25 nucleobases in length. In some embodiments, a base sequence is from about 15 to about 22 nucleobases in length. In some embodiments, a base sequence is about 10, 11, 12, 13, 14, 15, 16, 17, 18, 19, 20, 21, 22, 23, 24, or 25 nucleobases in length. In some embodiments, a base sequence is at least 12 nucleobases in length. In some embodiments, a base sequence is at least 13 nucleobases in length. In some embodiments, a base sequence is at least 14 nucleobases in length. In some embodiments, a base sequence is at least 15 nucleobases in length. In some embodiments, a base sequence is at least 16 nucleobases in length. In some embodiments, a base sequence is at least 17 nucleobases in length. In some embodiments, a base sequence is at least 18 nucleobases in length. In some embodiments, a base sequence is at least 19 nucleobases in length. In some embodiments, a base sequence is at least 20 nucleobases in length. In some embodiments, a base sequence is at least 21 nucleobases in length. In some embodiments, a base sequence is at least 22 nucleobases in length. In some embodiments, a base sequence is at least 23 nucleobases in length. In some embodiments, a base sequence is at least 24 nucleobases in length. In some embodiments, a base sequence is at least 25 nucleobases in length. In some embodiments, a base sequence is 15 nucleobases in length. In some embodiments, a base sequence is 16 nucleobases in length. In some embodiments, a base sequence is 17 nucleobases in length. In some embodiments, a base sequence is 18 nucleobases in length. In some embodiments, a base sequence is 19 nucleobases in length. In some embodiments, a base sequence is 20 nucleobases in length. In some embodiments, a base sequence is 21 nucleobases in length. In some embodiments, a base sequence is 22 nucleobases in length. In some embodiments, a base sequence is 23 nucleobases in length. In some embodiments, a base sequence is 24 nucleobases in length. In some embodiments, a base sequence is 25 nucleobases in length. In some other embodiments, a base sequence is at least 30 nucleobases in length. In some other embodiments, a base sequence is a duplex of complementary strands of at least 18 nucleobases in length. In some other embodiments, a base sequence is a duplex of complementary strands of at least 21 nucleobases in length. In some embodiments, each nucleobase independently comprises an optionally substituted monocyclic, bicyclic or polycyclic ring wherein at least one ring atom is nitrogen. In some embodiments, each nucleobase is independently optionally substituted adenine, cytosine, guanosine, thymine, or uracil, or an optionally substituted tautomer of adenine, cytosine, guanosine, thymine, or uracil.

### Certain Aspects of Non-Limiting Examples of USH2A Oligonucleotides

In some embodiments, an USH2A oligonucleotide comprises several regions, each of which independently comprises one or more consecutive nucleosides and optionally one or more internucleotidic linkages. In some embodiments, a region differs from its neighboring region(s) in that it contains one or more structural feature that are different from those corresponding structural features of its neighboring region(s). Example structural features include nucleobase modifications and patterns thereof, sugar modifications and patterns thereof, internucleotidic linkages and patterns thereof (which can be internucleotidic linkage types (e.g., phosphate, phosphorothioate, phosphorothioate triester, neutral internucleotidic linkage, etc.) and patterns thereof, linkage phosphorus modifications (backbone phosphorus modifications) and patterns thereof (e.g., pattern of -XLR¹ if internucleotidic linkages having the structure of formula I), backbone chiral center (linkage phosphorus) stereochemistry and patterns thereof [e.g., combination of Rp and/or Sp of chirally controlled internucleotidic linkages (sequentially from 5′ to 3′), optionally with non-chirally controlled internucleotidic linkages and/or natural phosphate linkages, if any (e.g., SSnXSSnXSSOSSSOSSSnXSS in Table A1)]. In some embodiments, a region comprises a chemical modification (e.g., a sugar modification, base modification, internucleotidic linkage, or stereochemistry of internucleotidic linkage) not present in its neighboring region(s). In some embodiments, a region lacks a chemical modification present in its neighboring regions(s).

In some embodiments, certain sugar modifications, e.g., 2′-MOE, provide more stability under certain conditions than other sugar modifications, e.g., 2′-OMe. In some embodiments, an USH2A oligonucleotides comprises one or more 2′-MOE modifications. In some embodiments, each nucleoside unit comprising a pyrimidine base (e.g., C, U, T, etc.) comprises a 2′-MOE modification.

In some embodiments, the present disclosure pertains to an USH2A oligonucleotide, wherein the majority of the sugars comprise 2′-F (e.g., 60%-100%, at least 60%, 65%, 70%, 75%, 80%, 85%, 90%, or 95%, or 100%). Non-limiting examples of such oligonucleotides include: WV-20891, WV-20892, WV-20902, WV-20908, WV-20988, WV-21008, WV-24297, WV-24368, WV-24376, WV-24366, WV-24375, WV-24360, WV-24298, WV-24381, WV-24382, WV-21100, WV-21105, and WV-20885.

In some embodiments, the present disclosure pertains to an USH2A oligonucleotide, wherein the majority of the sugars comprise 2′-F and a minority of the sugars comprise a different 2′-modification. Non-limiting examples of such oligonucleotides include: WV-20891, WV-20892, WV-20902, WV-20908, WV-20988, WV-21008, WV-24297, WV-24368, WV-24376, WV-24366, WV-24375, WV-24360, WV-24298, WV-24381, WV-24382, WV-21100, WV-21105, and WV-20885.

In some embodiments, the present disclosure pertains to an USH2A oligonucleotide, wherein the majority of the sugars comprise 2′-F and a minority of the sugars comprise a 2′-OMe. Non-limiting examples of such oligonucleotides include: WV-20891, WV-20892, WV-20902, WV-20908, WV-20988, WV-21008, WV-24297, WV-24368, WV-24376, WV-24366, WV-24375, WV-24360, WV-24298, WV-24381, WV-24382, WV-21100, WV-21105, and WV-20885.

In some embodiments, the present disclosure pertains to an USH2A oligonucleotide, wherein the majority of the sugars comprise 2′-MOE and a minority of the sugars comprise a 2′-F.

In some embodiments, the present disclosure pertains to an USH2A oligonucleotide, wherein the majority of the sugars comprise 2′-OMe and a minority of the sugars are independently bicyclic sugars. In some embodiments, the present disclosure pertains to an USH2A oligonucleotide, wherein the majority of the sugars comprise 2′-OMe and the minority of the sugars are independently bicyclic sugars.

In some embodiments, the present disclosure pertains to an USH2A oligonucleotide, wherein the majority of the sugars comprise 2′-OMe and at least one sugar is a bicyclic sugar and at least one sugar comprises 2′-OMe. In some embodiments, the present disclosure pertains to an USH2A oligonucleotide, wherein the majority of the sugars comprise 2′-OMe and at least one sugar is a bicyclic sugar and at least one sugar comprises 2′-OMe.

In some embodiments, the present disclosure pertains to an USH2A oligonucleotide, wherein the majority of the sugars are bicyclic sugars and at least one sugar is a bicyclic sugar and at least one sugar comprises 2′-OMe. In some embodiments, the present disclosure pertains to an USH2A oligonucleotide, wherein the majority of the sugars are independently bicyclic sugars and at least one sugar is a bicyclic sugar and at least one sugar comprises 2′-OMe.

In some embodiments, the present disclosure pertains to an USH2A oligonucleotide, wherein each sugar comprises 2′-OMe or a bicyclic sugar. In some embodiments, the present disclosure pertains to an USH2A oligonucleotide, wherein each sugar comprises 2′-OMe or a bicyclic sugar or a natural DNA sugar.

In some embodiments, the present disclosure pertains to an USH2A oligonucleotide, wherein each sugar is independently a bicyclic sugar or 2′-OMe or a natural DNA sugar.

In some embodiments, the present disclosure pertains to an USH2A oligonucleotide, which comprises a bicyclic sugar or 2′-MOE. In some embodiments, the present disclosure pertains to an USH2A oligonucleotide, which comprises a bicyclic sugar or 2′-MOE.

In some embodiments, the present disclosure pertains to an USH2A oligonucleotide, wherein the majority of the sugars are independently bicyclic sugars and the minority of the sugars comprise 2′-MOE. In some embodiments, the present disclosure pertains to an USH2A oligonucleotide, wherein the majority of the sugars comprise are independently bicyclic sugars and the minority of the sugars comprise 2′-MOE.

In some embodiments, the present disclosure pertains to an USH2A oligonucleotide, wherein the majority of the sugars are independently bicyclic sugars and at least one sugar comprises 2′-MOE and at least one sugar is a bicyclic sugar. In some embodiments, the present disclosure pertains to an USH2A oligonucleotide, wherein the majority of the sugars are independently bicyclic sugars and at least one sugar comprises 2′-MOE and at least one sugar is a bicyclic sugar.

In some embodiments, the present disclosure pertains to an USH2A oligonucleotide, wherein the majority of the sugars comprise 2′-MOE and at least one sugar is a bicyclic sugar. In some embodiments, the present disclosure pertains to an USH2A oligonucleotide, wherein the majority of the sugars comprise 2′-MOE and at least one sugar is a bicyclic sugar.

In some embodiments, the present disclosure pertains to an USH2A oligonucleotide, wherein each sugar is independently a bicyclic sugar or 2′-MOE. In some embodiments, the present disclosure pertains to an USH2A oligonucleotide, wherein each sugar is independently a bicyclic sugar or a 2′-MOE.

In some embodiments, the present disclosure pertains to an USH2A oligonucleotide, wherein each sugar comprises 2′-MOE or a bicyclic sugar or a natural DNA sugar.

In some embodiments, a bicyclic sugar is a LNA, a cEt or a BNA sugar.

In some embodiments, the present disclosure pertains to an USH2A oligonucleotide, which comprises 2′-OMe or 2′-F. In some embodiments, the present disclosure pertains to an USH2A oligonucleotide, which comprises 2′-OMe or 2′-F.

In some embodiments, the present disclosure pertains to an USH2A oligonucleotide, wherein the majority of the sugars comprise 2′-OMe and the minority of the sugars comprise 2′-F. In some embodiments, the present disclosure pertains to an USH2A oligonucleotide, wherein the majority of the sugars comprise 2′-OMe and the minority of the sugars comprise 2′-F.

In some embodiments, the present disclosure pertains to an USH2A oligonucleotide, wherein the majority of the sugars comprise 2′-OMe and at least one sugar comprises 2′-F and at least one sugar comprises 2′-OMe. In some embodiments, the present disclosure pertains to an USH2A oligonucleotide, wherein the majority of the sugars comprise 2′-OMe and at least one sugar is 2′-F and at least one sugar comprises 2′-OMe.

In some embodiments, the present disclosure pertains to an USH2A oligonucleotide, wherein the majority of the sugars comprise 2′-F and at least two sugars comprise 2′-F and at least two sugars comprise 2′-OMe. In some embodiments, the present disclosure pertains to an USH2A oligonucleotide, wherein the majority of the sugars comprise 2′-F and at least two sugars comprise 2′-F and at least two sugars comprise 2′-OMe.

In some embodiments, the present disclosure pertains to an USH2A oligonucleotide, wherein each sugar of the oligonucleotide comprises 2′-OMe or 2′-F. In some embodiments, the present disclosure pertains to an USH2A oligonucleotide, wherein each sugar of the oligonucleotide comprises 2′-OMe and a 2′-F.

In some embodiments, the present disclosure pertains to an USH2A oligonucleotide, wherein each sugar comprises 2′-F or 2′-OMe or a DNA sugar.

In some embodiments, the present disclosure pertains to an USH2A oligonucleotide, which comprises 2′-F or 2′-MOE. In some embodiments, the present disclosure pertains to an USH2A oligonucleotide, which comprises 2′-F or 2′-MOE.

In some embodiments, the present disclosure pertains to an USH2A oligonucleotide, wherein the majority of the sugars comprise 2′-F and the minority of the sugars comprise 2′-MOE. In some embodiments, the present disclosure pertains to an USH2A oligonucleotide, wherein the majority of the sugars comprise 2′-F and the minority of the sugars comprise 2′-MOE.

In some embodiments, the present disclosure pertains to an USH2A oligonucleotide, wherein the majority of the sugars comprise 2′-F and at least one sugar comprises 2′-MOE and at least one sugar comprises 2′-F. In some embodiments, the present disclosure pertains to an USH2A oligonucleotide, wherein the majority of the sugars comprise 2′-F and at least one sugar comprises 2′-MOE.

In some embodiments, the present disclosure pertains to an USH2A oligonucleotide, wherein the majority of the sugars comprise 2′-MOE and at least one sugar comprises 2′-F. In some embodiments, the present disclosure pertains to an USH2A oligonucleotide, wherein the majority of the sugars comprise 2′-MOE and at least one sugar comprises 2′-F.

In some embodiments, the present disclosure pertains to an USH2A oligonucleotide, wherein each sugar of the oligonucleotide comprises 2′-MOE or 2′-F.

In some embodiments, each sugar of a provided oligonucleotide is modified. In some embodiments, each sugar of a provided oligonucleotide is modified, wherein the modification is selected from 2′-F and 2′-OR. In some embodiments, R is methyl.

In some embodiments, the pattern of sugars in a stereodefined (e.g., chirally controlled or stereopure) USH2A oligonucleotide is or comprises a sequence of: D [symbol] D [symbol] , D [symbol] D, DDD [symbol] [symbol] , DD [symbol] D, D [symbol] [symbol] [symbol] DD, [symbol] DDD, [symbol] [symbol] [symbol] [symbol] DD [symbol] [symbol] , [symbol] DD, [symbol] [symbol] [symbol] [symbol] [symbol] D [symbol] D, D [symbol] [symbol] [symbol] [symbol] [symbol] D [symbol] , DDDD, [symbol] , DD [symbol] [symbol] [symbol] [symbol] [symbol] D, DD [symbol] D, D [symbol] , [symbol] [symbol] [symbol] [symbol] [symbol] D [symbol] , [symbol] D [symbol] [symbol] [symbol] [symbol] [symbol] , [symbol] D, [symbol] D [symbol] [symbol] [symbol] [symbol] [symbol] , [symbol] D [symbol] [symbol] [symbol] [symbol] [symbol] D, [symbol] D [symbol] [symbol] [symbol] [symbol] [symbol] DD, [symbol] D [symbol] [symbol] [symbol] [symbol] [symbol] DD [symbol] , [symbol] D [symbol] [symbol] [symbol] [symbol] [symbol] DD [symbol] D, [symbol] D [symbol] [symbol] [symbol] [symbol] [symbol] DD [symbol] D [symbol] [symbol] [symbol] [symbol] [symbol] , [symbol] D [symbol] [symbol] [symbol] [symbol] [symbol] DD [symbol] D [symbol] [symbol] [symbol] [symbol] [symbol] D, [symbol] D [symbol] [symbol] [symbol] [symbol] [symbol] DD [symbol] D [symbol] [symbol] [symbol] [symbol] [symbol] DD, [symbol] D [symbol] [symbol] [symbol] [symbol] [symbol] DD [symbol] D [symbol] [symbol] [symbol] [symbol] [symbol] DD [symbol] , [symbol] , D [symbol] , DD [symbol] , [symbol] [symbol] [symbol] [symbol] [symbol] DD [symbol] , D [symbol] [symbol] [symbol] [symbol] [symbol] DD [symbol] , [symbol] [symbol] [symbol] [symbol] [symbol] D [symbol] DD [symbol] , D [symbol] [symbol] [symbol] [symbol] [symbol] D [symbol] DD [symbol] , DD [symbol] [symbol] [symbol] [symbol] [symbol] D [symbol] DD [symbol] , DD [symbol] [symbol] [symbol] [symbol] [symbol] D [symbol] DD [symbol] , D [symbol] [symbol] [symbol] [symbol] [symbol] DD [symbol] D [symbol] [symbol] [symbol] [symbol] [symbol] DD [symbol] , [symbol] [symbol] [symbol] [symbol] [symbol] D [symbol] DD [symbol] [symbol] [symbol] [symbol] [symbol] D [symbol] DD [symbol] , [symbol] D [symbol] [symbol] [symbol] [symbol] [symbol] DD [symbol] D [symbol] [symbol] [symbol] [symbol] [symbol] DD [symbol] , wherein D is 2′- deoxyribose (unmodified DNA sugar) and [symbol] [symbol] [symbol] [symbol] [symbol] is a sugar which is not a 2′-deoxyribose.

In some embodiments, the pattern of sugars in a stereodefined oligonucleotide is or comprises a sequence of: DLDL, DLLD, DDDL, DDLD, DLDD, LDDD, LDDL, LLDD, LDLD, DLDL, DDDD, LLLL, DDLD, DDLL, DLLL, LDLL, LLDL, LLLD, LLDL, LLDLD, LLDLDD, LLDLDDL, LLDLDDLD, LLDLDDLDL, LLDLDDLDLD, LLDLDDLDLDD, LLDLDDLDLDDL, LL, DLL, DDLL, LDDLL, DLDDLL, LDLDDLL, DLDLDDLL, DDLDLDDLL, DDLDLDDLL, DLDDLDLDDLL, LDLDDLDLDDLL, LLDLDDLDLDDLL, LLDLDDLDLDDLL, wherein L is LNA sugar modification, and D is 2′-deoxyribose (unmodified DNA sugar).

Among other things, the present disclosure encompasses the recognition that 2′-modifications and/or modified internucleotidic linkages can be utilized either individually or in combination to fine-tune properties, e.g., stability, and/or activities of oligonucleotides. In some embodiments, modified (non-natural) internucleotidic linkages (which are not natural phosphate linkage or salt forms thereof), such as phosphorothioate linkages (phosphorothioate diester linkages), can be utilized to improve properties, e.g., stability (e.g., by using Sp phosphorothioate linkages), of an oligonucleotide. In some embodiments, in an USH2A oligonucleotide a particular modified internucleotidic linkage can be used in combination with a particular sugar to achieve desired properties and/or activities.

In some embodiments, an USH2A oligonucleotide comprises a modified internucleotidic linkage. In some embodiments, a modified internucleotidic linkage is a phosphorothioate linage. In some embodiments, a modified internucleotidic linkage is a chirally controlled internucleotidic linkage. In some embodiments, a modified internucleotidic linkage is a chirally controlled internucleotidic linkage wherein the linkage phosphorus is of Sp configuration. In some embodiments, a modified internucleotidic linkage is a chirally controlled internucleotidic linkage wherein the linkage phosphorus is of Rp configuration. In some embodiments, a modified internucleotidic linkage is a Sp phosphorothioate linkage. In some embodiments, a modified internucleotidic linkage is a Rp phosphorothioate linkage.

In some embodiments, an USH2A oligonucleotide comprises one or more, e.g., 1, 2, 3, 4, 5, 6 or more, natural phosphate linkages. In some embodiments, the number of natural phosphate linkage is 1. In some embodiments, the number of natural phosphate linkages is 2. In some embodiments, the number of natural phosphate linkages is 3. In some embodiments, the number of natural phosphate linkages is 4. In some embodiments, the number of natural phosphate linkages is 5. In some embodiments, the number of natural phosphate linkages is 6. In some embodiments, 2 natural phosphate linkages are consecutive. In some embodiments, 3 natural phosphate linkages are consecutive. In some embodiments, 4 natural phosphate linkages are consecutive. In some embodiments, 5 natural phosphate linkages are consecutive. In some embodiments, 6 natural phosphate linkages are consecutive. In some embodiments, a modified internucleotidic linkage is Sp. In some embodiments, a modified internucleotidic linkage is Rp. In some embodiments, a modified internucleotidic linkage is a phosphorothioate linkage. In some embodiments, a modified internucleotidic linkage is a Sp phosphorothioate linkage. In some embodiments, a modified internucleotidic linkage is a Rp phosphorothioate linkage.

In some embodiments, a modified internucleotidic linkage is chirally controlled and is Sp. In some embodiments, a modified internucleotidic linkage is chirally controlled and is Rp. In some embodiments, a modified internucleotidic linkage is a chirally controlled Sp phosphorothioate internucleotidic linkage. In some embodiments, a modified internucleotidic linkage is a chirally controlled Rp phosphorothioate internucleotidic linkage. Among other things, the present disclosure demonstrates that Rp internucleotidic linkages can be utilized as the 5′-end and/or the 3′-end internucleotidic linkages despite that in some cases they are less stable than corresponding Sp internucleotidic linkages, e.g., toward nuclease activities.

In some embodiments, each internucleotidic linkage linking two sugars comprising 2′-OR′, wherein R′ is optionally substituted alkyl, is independently a natural phosphate linkage, except the 5′-end and the 3′-end internucleotidic linkages, which are independently optionally chirally controlled modified internucleotidic linkages (e.g., in some embodiments, chirally controlled phosphorothioate internucleotidic linkages).

In some embodiments, internucleotidic linkages that are not modified internucleotidic linkages of Sp configuration (e.g., each and every pair of two natural phosphate linkages, two modified internucleotidic linkages of Rp configuration, or one natural phosphate linkage and one modified internucleotidic linkage) are separated by two or more modified internucleotidic linkages of Sp configuration. For example, in RSSRSSSSRSS, the Rp internucleotidic linkages (R) are separated by at least two Sp internucleotidic linkages (S). In some embodiments, a modified internucleotidic linkage is of Formula I as described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612. In some embodiments, a modified internucleotidic linkage is a phosphorothioate linkage.

In some embodiments, the present disclosure pertains to an USH2A oligonucleotide which comprises at least one 2′-MOE.

In some embodiments, the present disclosure pertains to an USH2A oligonucleotide which comprises at least one 2′-OMe. Non-limiting examples of such an USH2A oligonucleotide include but are not limited to: WV-24368, WV-24376, WV-24366, WV-24375, WV-24381, WV-24382, WV-21100, and WV-21105.

In some embodiments, the present disclosure pertains to an USH2A oligonucleotide which comprises at least one phosphorothioate internucleotidic linkage. Non-limiting examples of such an USH2A oligonucleotide include but are not limited to: WV-20902, WV-20908, WV-20988, WV-21008, and WV-24297.

In some embodiments, the present disclosure pertains to an USH2A oligonucleotide which comprises at least one phosphorothioate internucleotidic linkage which is chirally controlled. Non-limiting examples of such an USH2A oligonucleotide include but are not limited to: WV-20902, WV-20908, WV-20988, WV-21008, and WV-24297.

In some embodiments, the present disclosure pertains to an USH2A oligonucleotide which comprises at least one phosphorothioate internucleotidic linkage which is chirally controlled and in the Sp configuration. Non-limiting examples of such an USH2A oligonucleotide include but are not limited to: WV-20902, WV-20908, WV-20988, WV-21008, and WV-24297.

In some embodiments, the present disclosure pertains to an USH2A oligonucleotide, in which the majority of the internucleotidic linkages are phosphorothioate internucleotidic linkages. Non-limiting examples of such an USH2A oligonucleotide include but are not limited to: WV-20902, WV-20908, WV-20988, WV-21008, and WV-24297.

In some embodiments, the present disclosure pertains to an USH2A oligonucleotide, in which the majority of the internucleotidic linkages are phosphorothioate internucleotidic linkages which is chirally controlled. Non-limiting examples of such an USH2A oligonucleotide include but are not limited to: WV-20902, WV-20908, WV-20988, WV-21008, and WV-24297.

In some embodiments, the present disclosure pertains to an USH2A oligonucleotide, in which the majority of the internucleotidic linkages are phosphorothioate internucleotidic linkage which are chirally controlled and in the Sp configuration. Non-limiting examples of such an USH2A oligonucleotide include but are not limited to: WV-20902, WV-20908, WV-20988, WV-21008, and WV-24297.

In some embodiments, the present disclosure pertains to an USH2A oligonucleotide, in which all of the internucleotidic linkages are phosphorothioate internucleotidic linkages which are chirally controlled and in the Sp configuration. Non-limiting examples of such an USH2A oligonucleotide include but are not limited to: WV-20902, WV-20908, WV-20988, WV-21008, and WV-24297.

In some embodiments, the present disclosure pertains to an USH2A oligonucleotide which comprises at least one neutral or non-negatively charged internucleotidic linkage. Non-limiting examples of such an USH2A oligonucleotide include but are not limited to: WV-24368, WV-24376, WV-24366, WV-24375, WV-24381, WV-24382, WV-21100, and WV-21105.

In some embodiments, the present disclosure pertains to an USH2A oligonucleotide which comprises a chirally-controlled neutral or non-negatively charged internucleotidic linkage.

In some embodiments, an USH2A oligonucleotide comprises at least three different types of internucleotidic linkages. Non-limiting examples of such an USH2A oligonucleotide include but are not limited to: WV-24368, WV-24366, and WV-21105.

In some embodiments, an USH2A oligonucleotide comprises: at least one natural phosphate internucleotidic linkage; at least one phosphorothioate; and at least one neutral or non-negatively charged internucleotidic linkage. Non-limiting examples of such an USH2A oligonucleotide include but are not limited to: WV-24368, WV-24366, and WV-21105.

In some embodiments, an USH2A oligonucleotide comprises: at least one natural phosphate internucleotidic linkage; at least one phosphorothioate which is chirally controlled; and at least one neutral or non-negatively charged internucleotidic linkage. Non-limiting examples of such an USH2A oligonucleotide include but are not limited to: WV-24368, WV-24366, and WV-21105.

In some embodiments, an USH2A oligonucleotide comprises: at least one natural phosphate internucleotidic linkage; at least one phosphorothioate; and at least one neutral or non-negatively charged internucleotidic linkage which is chirally controlled.

In some embodiments, an USH2A oligonucleotide comprises: at least one natural phosphate internucleotidic linkage; at least one phosphorothioate which is chirally controlled; and at least one neutral or non-negatively charged internucleotidic linkage which is chirally controlled.

In some embodiments, the present disclosure pertains to an USH2A oligonucleotide which comprises at least 2 different types of sugars. Non-limiting examples of such an oligonucleotide include but are not limited to: WV-20891, WV-20892, WV-20902, WV-20908, WV-20988, WV-21008, WV-24297, WV-24368, WV-24376, WV-24366, WV-24375, WV-24360, WV-24298, WV-24381, WV-24382, WV-21100, WV-21105, and WV-20885.

In some embodiments, the present disclosure pertains to an USH2A oligonucleotide which comprises 2′-DNA sugar (a natural 2′-deoxyribose) and a sugar comprising 2′-modification.

In some embodiments, the present disclosure pertains to an USH2A oligonucleotide which comprises 2′-DNA sugar (a natural 2′-deoxyribose) and a 2′-OMe sugar.

In some embodiments, an USH2A oligonucleotide comprises at least one natural 2′-deoxyribose sugar (unmodified DNA sugar), at least one LNA sugar and at least one 2′-MOE sugar.

In some embodiments, the present disclosure pertains to an USH2A oligonucleotide which comprises a natural 2′-deoxyribose (unmodified DNA sugar), a LNA sugar and 2′-MOE sugar.

In some embodiments, an USH2A oligonucleotide comprises at least one natural 2′-deoxyribose (unmodified DNA sugar), at least one LNA sugar and at least one 2′-OMe sugar.

In some embodiments, the present disclosure pertains to an USH2A oligonucleotide which comprises a natural 2′-deoxyribose (unmodified DNA sugar), a LNA sugar and 2′-OMe sugar.

In some embodiments, the present disclosure pertains to an USH2A oligonucleotide which comprises at least 3 different types of sugars (e.g., selected from unmodified sugars and modified sugars with various modifications).

In some embodiments, the present disclosure pertains to an USH2A oligonucleotide, which comprises at least one 2′-F sugar or at least one 2′-MOE sugar.

In some embodiments, the present disclosure pertains to an USH2A oligonucleotide which comprises one or more (1, 2, 3, 4, 5, 6, 7, 8, 9, or 10) LNA sugars.

In some embodiments, the present disclosure pertains to an USH2A oligonucleotide which comprises one or more 2′-MOE sugars and one or more LNA sugars.

In some embodiments, the present disclosure pertains to an USH2A oligonucleotide which comprises one or more LNA sugars.

In some embodiments, the present disclosure pertains to an USH2A oligonucleotide, which comprises one or more LNA sugars and one or more 2′-MOE sugars or one or more LNA sugars and one or more 2′-OMe sugars.

In some embodiments, the present disclosure pertains to an USH2A oligonucleotide, which comprises 2′-MOE and 2′-F sugars, or a 2′-MOE sugar.

In some embodiments, the present disclosure pertains to an USH2A oligonucleotide, which comprises a natural 2′-deoxyribose (unmodified DNA sugar), a LNA sugar, and a 2′-MOE sugar.

In some embodiments, the present disclosure pertains to an USH2A oligonucleotide, which comprises at least 3 different types of sugars.

In some embodiments, the present disclosure pertains to an USH2A oligonucleotide which comprises a natural 2′-deoxyribose (unmodified DNA sugar) and at least 1 modified sugar (compared to 2′-deoxyribose (unmodified DNA sugar)).

In some embodiments, the present disclosure pertains to an USH2A oligonucleotide which comprises a natural 2′-deoxyribose (unmodified DNA sugar) and at least 2 sugar modifications.

In some embodiments, the present disclosure pertains to an USH2A oligonucleotide, which comprises at least one 2′-MOE sugar or at least one 2′-OMe sugar.

In some embodiments, the present disclosure pertains to an USH2A oligonucleotide, which comprises a 2′-F sugar and a 2′-OMe sugar.

In some embodiments, the present disclosure pertains to an USH2A oligonucleotide, which comprises a natural 2′-deoxyribose (unmodified DNA sugar) and at least one modified sugar.

In some embodiments, the present disclosure pertains to an USH2A oligonucleotide, which comprises a natural 2′-deoxyribose (unmodified DNA sugar) and at least two modified sugars.

### Internucleotidic Linkages

In some embodiments, oligonucleotides comprise base modifications, sugar modifications, and/or internucleotidic linkage modifications. Various internucleotidic linkages can be utilized in accordance with the present disclosure to link units comprising nucleobases, e.g., nucleosides. In some embodiments, USH2A oligonucleotides comprise both one or more modified internucleotidic linkages and one or more natural phosphate linkages. As widely known by those skilled in the art, natural phosphate linkages are widely found in natural DNA and RNA molecules; they have the structure of —OP(O)(OH)O—, connect sugars in the nucleosides in DNA and RNA, and may be in various salt forms, for example, at physiological pH (about 7.4), natural phosphate linkages are predominantly exist in salt forms with the anion being —OP(O)(O⁻)O—. A modified internucleotidic linkage, or a non-natural phosphate linkage, is an internucleotidic linkage that is not natural phosphate linkage or a salt form thereof. Modified internucleotidic linkages, depending on their structures, may also be in their salt forms. For example, as appreciated by those skilled in the art, phosphorothioate internucleotidic linkages which have the structure of —OP(O)(SH)O— may be in various salt forms, e.g., at physiological pH (about 7.4) with the anion being —OP(O)(S⁻)O—.

In some embodiments, an oligonucleotide comprises an internucleotidic linkage which is a modified internucleotidic linkage, e.g., phosphorothioate, phosphorodithioate, methylphosphonate, phosphoroamidate, thiophosphate, 3′-thiophosphate, or 5′-thiophosphate.

In some embodiments, a modified internucleotidic linkage is a chiral internucleotidic linkage which comprises a chiral linkage phosphorus. In some embodiments, a chiral internucleotidic linkage is a phosphorothioate linkage. In some embodiments, a chiral internucleotidic linkage is a non-negatively charged internucleotidic linkage. In some embodiments, a chiral internucleotidic linkage is a neutral internucleotidic linkage. In some embodiments, a chiral internucleotidic linkage is chirally controlled with respect to its chiral linkage phosphorus. In some embodiments, a chiral internucleotidic linkage is stereochemically pure with respect to its chiral linkage phosphorus. In some embodiments, a chiral internucleotidic linkage is not chirally controlled. In some embodiments, a pattern of backbone chiral centers comprises or consists of positions and linkage phosphorus configurations of chirally controlled internucleotidic linkages (Rp or Sp) and positions of achiral internucleotidic linkages (e.g., natural phosphate linkages).

Oligonucleotides, e.g., USH2A oligonucleotides, can comprise various numbers of natural phosphate linkages, e.g., 1-50, 1-40, 1-30, 1-25, 1-20, 1-10, 1-5, or 1, 2, 3, 4, 5, 6, 7, 8, 9, 10, 11, 12, 13, 14, 15, 16, 17, 18, 19, 20 or more. In some embodiments, one or more (e.g., 1-50, 1-40, 1-30, 1-25, 1-20, 1-10, 1-5, or 1, 2, 3, 4, 5, 6, 7, 8, 9, 10, 11, 12, 13, 14, 15, 16, 17, 18, 19, 20 or more) of the natural phosphate linkages in an oligonucleotide are consecutive. In some embodiments, provided oligonucleotides comprise no natural phosphate linkages. In some embodiments, provided oligonucleotides comprise one natural phosphate linkage. In some embodiments, provided oligonucleotides comprise 1 to 30 or more natural phosphate linkages.

In some embodiments, an oligonucleotide comprises a modified internucleotidic linkage (e.g., a modified internucleotidic linkage having the structure of Formula I, I-a, I-b, or I-c, I-n-1, I-n-2, I-n-3, I-n-4, II, II-a-1, II-a-2, II-b-1, II-b-2, II-c-1, II-c-2, II-d-1, II-d-2, etc., or a salt form thereof) as described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612 the internucleotidic linkages (e.g., those of Formula I, I-a, I-b, or I-c, I-n-1, I-n-2, I-n-3, I-n-4, II, II-a-1, II-a-2, II-b-1, II-b-2, II-c-1, II-c-2, II-d-1, II-d-2, etc.) of each of which are independently incorporated herein by reference. In some embodiments, a modified internucleotidic linkage is a non-negatively charged internucleotidic linkage. In some embodiments, provided oligonucleotides comprise one or more non-negatively charged internucleotidic linkages. In some embodiments, a non-negatively charged internucleotidic linkage is a positively charged internucleotidic linkage. In some embodiments, a non-negatively charged internucleotidic linkage is a neutral internucleotidic linkage. In some embodiments, the present disclosure provides oligonucleotides comprising one or more neutral internucleotidic linkages. In some embodiments, a non-negatively charged internucleotidic linkage or a neutral internucleotidic linkage (e.g., one of Formula I-n-1, I-n-2, I-n-3, I-n-4, II, II-a-1, II-a-2, II-b-1, II-b-2, II-c-1, II-c-2, II-d-1, II-d-2, etc.) is as described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612. In some embodiments, a non-negatively charged internucleotidic linkage or neutral internucleotidic linkage is one of Formula I-n-1, I-n-2, I-n-3, I-n-4, II, II-a-1, II-a-2, II-b-1, II-b-2, II-c-1, II-c-2, II-d-1, II-d-2, etc. as described in WO 2018/223056, WO 2019/032607, WO 2019/075357, WO 2019/032607, WO 2019/075357, WO 2019/200185, WO 2019/217784, and/or WO 2019/032612, such internucleotidic linkages of each of which are independently incorporated herein by reference.

In some embodiments, a non-negatively charged internucleotidic linkage can improve the delivery and/or activity (e.g., ability to increase the level of skipping of a deleterious exon in a mutant USH2A gene transcript or a gene product thereof) of an oligonucleotide.

In some embodiments, a non-negatively charged internucleotidic linkage has the structure of —OP(═W)(—N═C(R″)₂)—O— or —OP(═W)(—N(R″)₂)—O—, wherein:
W is O or S;
each R″ is independently R′ or —N(R′)₂;
each R′ is independently —R, —C(O)R, —C(O)OR, or —S(O)₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or:
two R groups are optionally and independently taken together to form a covalent bond, or:
two or more R groups on the same atom are optionally and independently taken together with the atom to form an optionally substituted, 3-30 membered monocyclic, bicyclic or polycyclic ring having, in addition to the atom, 0-10 heteroatoms, or:
two or more R groups on two or more atoms are optionally and independently taken together with their intervening atoms to form an optionally substituted, 3-30 membered monocyclic, bicyclic or polycyclic ring having, in addition to the intervening atoms, 0-10 heteroatoms.

In some embodiments, W is O. In some embodiments, W is S.

In some embodiments, R″ is R′. In some embodiments, R″ is —N(R′)₂.

In some embodiments, a non-negatively charged internucleotidic linkage has the structure of —OP(═O)(—N═C(N(R′)₂)₂—O—. In some embodiments, a R′ group of one N(R′)₂ is R, a R′ group of the other N(R′)₂ is R, and the two R groups are taken together with their intervening atoms to form an optionally substituted ring, e.g., a 5-membered ring as in n001. In some embodiments, each R′ is independently R, wherein each R is independently optionally substituted C₁₋₆ aliphatic.

In some embodiments, a non-negatively charged internucleotidic linkage has the structure of —OP(═W)(—N(R′)₂)—O—.

In some embodiments, R′ is R. In some embodiments, R′ is H. In some embodiments, R′ is —C(O)R. In some embodiments, R′ is —C(O)OR. In some embodiments, R′ is —S(O)₂R.

In some embodiments, R″ is —NHR′. In some embodiments, —N(R′)₂ is —NHR′.

As described herein, some embodiments, R is H. In some embodiments, R is optionally substituted C₁₋₆ aliphatic. In some embodiments, R is optionally substituted C₁₋₆ alkyl. In some embodiments, R is methyl. In some embodiments, R is substituted methyl. In some embodiments, R is ethyl. In some embodiments, R is substituted ethyl.

In some embodiments, as described herein, a non-negatively charged internucleotidic linkage is a neutral internucleotidic linkage.

In some embodiments, a modified internucleotidic linkage (e.g., a non-negatively charged internucleotidic linkage) comprises optionally substituted triazolyl. In some embodiments, a modified internucleotidic linkage (e.g., a non-negatively charged internucleotidic linkage) comprises optionally substituted alkynyl. In some embodiments, a modified internucleotidic linkage comprises a triazole or alkyne moiety. In some embodiments, a triazole moiety, e.g., a triazolyl group, is optionally substituted. In some embodiments, a triazole moiety, e.g., a triazolyl group) is substituted. In some embodiments, a triazole moiety is unsubstituted. In some embodiments, a modified internucleotidic linkage comprises an optionally substituted cyclic guanidine moiety. In some embodiments, a modified internucleotidic linkage comprises an optionally substituted cyclic guanidine moiety and has the structure of:

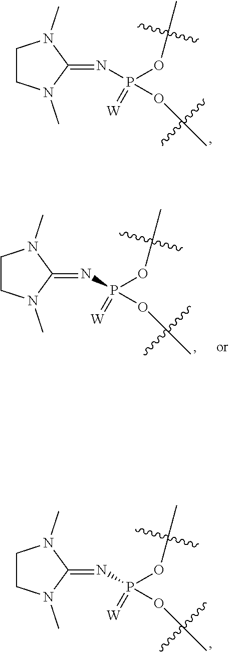

wherein W is O or S. In some embodiments, W is O. In some embodiments, W is S. In some embodiments, a non-negatively charged internucleotidic linkage is stereochemically controlled.

In some embodiments, a non-negatively charged internucleotidic linkage or a neutral internucleotidic linkage is an internucleotidic linkage comprising a triazole moiety. In some embodiments, a non-negatively charged internucleotidic linkage or a non-negatively charged internucleotidic linkage comprises an optionally substituted triazolyl group. In some embodiments, an internucleotidic linkage comprising a triazole moiety (e.g., an optionally substituted triazolyl group) has the structure of

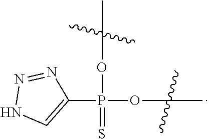 [SMILES: *OP(=S)(O*)c1c[nH]nn1]

In some embodiments, an internucleotidic linkage comprising a triazole moiety has the structure of

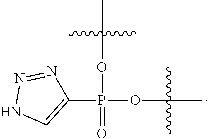 [SMILES: *OP(=O)(O*)c1c[nH]nn1]

In some embodiments, an internucleotidic linkage, e.g., a non-negatively charged internucleotidic linkage, a neutral internucleotidic linkage, comprises a cyclic guanidine moiety. In some embodiments, an internucleotidic linkage comprising a cyclic guanidine moiety has the structure of

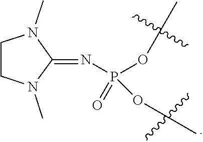 [SMILES: *OP(=O)(N=C1N(C)CCN1C)O*]

In some embodiments, a non-negatively charged internucleotidic linkage, or a neutral internucleotidic linkage, is or comprising a structure selected from

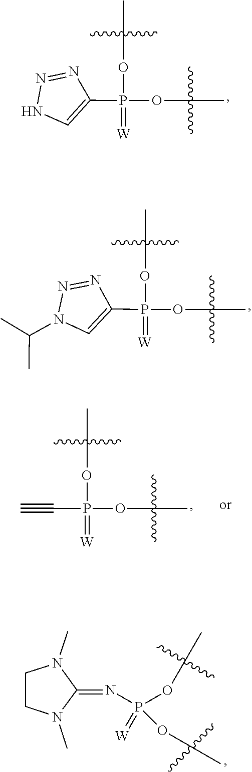

wherein W is O or S.

In some embodiments, an internucleotidic linkage comprises a Tmg group

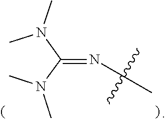 [SMILES: *N=C(N(C)C)N(C)C]

In some embodiments, an internucleotidic linkage comprises a Tmg group and has the structure of

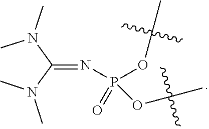 [SMILES: *OP(=O)(N=C(N(C)C)N(C)C)O*]

(the “Tmg internucleotidic linkage”). In some embodiments, neutral internucleotidic linkages include internucleotidic linkages of PNA and PMO, and an Tmg internucleotidic linkage.

In some embodiments, a non-negatively charged internucleotidic linkage comprises an optionally substituted 3-20 membered heterocyclyl or heteroaryl group having 1-10 heteroatoms. In some embodiments, a non-negatively charged internucleotidic linkage comprises an optionally substituted 3-20 membered heterocyclyl or heteroaryl group having 1-10 heteroatoms, wherein at least one heteroatom is nitrogen. In some embodiments, such a heterocyclyl or heteroaryl group is of a 5-membered ring. In some embodiments, such a heterocyclyl or heteroaryl group is of a 6-membered ring.

In some embodiments, a non-negatively charged internucleotidic linkage comprises an optionally substituted 5-20 membered heteroaryl group having 1-10 heteroatoms. In some embodiments, a non-negatively charged internucleotidic linkage comprises an optionally substituted 5-20 membered heteroaryl group having 1-10 heteroatoms, wherein at least one heteroatom is nitrogen. In some embodiments, a non-negatively charged internucleotidic linkage comprises an optionally substituted 5-6 membered heteroaryl group having 1-4 heteroatoms, wherein at least one heteroatom is nitrogen. In some embodiments, a non-negatively charged internucleotidic linkage comprises an optionally substituted 5-membered heteroaryl group having 1-4 heteroatoms, wherein at least one heteroatom is nitrogen. In some embodiments, a heteroaryl group is directly bonded to a linkage phosphorus. In some embodiments, a non-negatively charged internucleotidic linkage comprises an optionally substituted 5-20 membered heterocyclyl group having 1-10 heteroatoms. In some embodiments, a non-negatively charged internucleotidic linkage comprises an optionally substituted 5-20 membered heterocyclyl group having 1-10 heteroatoms, wherein at least one heteroatom is nitrogen. In some embodiments, a non-negatively charged internucleotidic linkage comprises an optionally substituted 5-6 membered heterocyclyl group having 1-4 heteroatoms, wherein at least one heteroatom is nitrogen. In some embodiments, a non-negatively charged internucleotidic linkage comprises an optionally substituted 5-membered heterocyclyl group having 1-4 heteroatoms, wherein at least one heteroatom is nitrogen. In some embodiments, at least two heteroatoms are nitrogen. In some embodiments, a heterocyclyl group is directly bonded to a linkage phosphorus. In some embodiments, a heterocyclyl group is bonded to a linkage phosphorus through a linker, e.g., ═N— when the heterocyclyl group is part of a guanidine moiety who directed bonded to a linkage phosphorus through its ═N—. In some embodiments, a non-negatively charged internucleotidic linkage comprises an optionally substituted

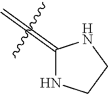 [SMILES: *=C1NCCN1]

group. In some embodiments, a non-negatively charged internucleotidic linkage comprises an substituted

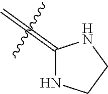 [SMILES: *=C1NCCN1]

group. In some embodiments, a non-negatively charged internucleotidic linkage comprises a

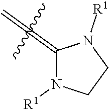

group. In some embodiments, each R¹ is independently optionally substituted C₁₋₆ alkyl. In some embodiments, each R¹ is independently methyl.

In some embodiments, an oligonucleotide comprises different types of internucleotidic phosphorus linkages. In some embodiments, a chirally controlled oligonucleotide comprises at least one natural phosphate linkage and at least one modified (non-natural) internucleotidic linkage. In some embodiments, an oligonucleotide comprises at least one natural phosphate linkage and at least one phosphorothioate. In some embodiments, an oligonucleotide comprises at least one non-negatively charged internucleotidic linkage. In some embodiments, an oligonucleotide comprises at least one natural phosphate linkage and at least one non-negatively charged internucleotidic linkage. In some embodiments, an oligonucleotide comprises at least one phosphorothioate internucleotidic linkage and at least one non-negatively charged internucleotidic linkage. In some embodiments, an oligonucleotide comprises at least one phosphorothioate internucleotidic linkage, at least one natural phosphate linkage, and at least one non-negatively charged internucleotidic linkage. In some embodiments, oligonucleotides comprise one or more, e.g., 1-50, 1-40, 1-30, 1-20, 1-15, 1-10, 1, 2, 3, 4, 5, 6, 7, 8, 9, 10, 11, 12, 13, 14, 15, 16, 17, 18, 19, 20 or more non-negatively charged internucleotidic linkages. In some embodiments, a non-negatively charged internucleotidic linkage is not negatively charged in that at a given pH in an aqueous solution less than 50%, 40%, 40%, 30%, 20%, 10%, 5%, or 1% of the internucleotidic linkage exists in a negatively charged salt form. In some embodiments, a pH is about pH 7.4. In some embodiments, a pH is about 4-9. In some embodiments, the percentage is less than 10%. In some embodiments, the percentage is less than 5%. In some embodiments, the percentage is less than 1%. In some embodiments, an internucleotidic linkage is a non-negatively charged internucleotidic linkage in that the neutral form of the internucleotidic linkage has no pKa that is no more than about 1, 2, 3, 4, 5, 6, or 7 in water. In some embodiments, no pKa is 7 or less. In some embodiments, no pKa is 6 or less. In some embodiments, no pKa is 5 or less. In some embodiments, no pKa is 4 or less. In some embodiments, no pKa is 3 or less. In some embodiments, no pKa is 2 or less. In some embodiments, no pKa is 1 or less. In some embodiments, pKa of the neutral form of an internucleotidic linkage can be represented by pKa of the neutral form of a compound having the structure of CH₃— the internucleotidic linkage —CH₃. For example, pKa of

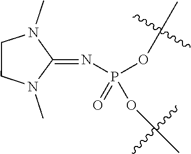 [SMILES: *OP(=O)(N=C1N(C)CCN1C)O*]

can be represented by pKa

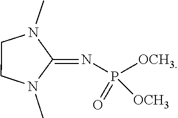

In some embodiments, a non-negatively charged internucleotidic linkage is a neutral internucleotidic linkage. In some embodiments, a non-negatively charged internucleotidic linkage is a positively-charged internucleotidic linkage. In some embodiments, a non-negatively charged internucleotidic linkage comprises a guanidine moiety. In some embodiments, a non-negatively charged internucleotidic linkage comprises a heteroaryl base moiety. In some embodiments, a non-negatively charged internucleotidic linkage comprises a triazole moiety. In some embodiments, a non-negatively charged internucleotidic linkage comprises an alkynyl moiety.

Without wishing to be bound by any particular theory, the present disclosure notes that a neutral internucleotidic linkage can be more hydrophobic than a phosphorothioate internucleotidic linkage (PS), which can be more hydrophobic than a natural phosphate linkage (PO). Typically, unlike a PS or PO, a neutral internucleotidic linkage bears less charge. Without wishing to be bound by any particular theory, the present disclosure notes that incorporation of one or more neutral internucleotidic linkages into an oligonucleotide may increase oligonucleotides' ability to be taken up by a cell and/or to escape from endosomes. Without wishing to be bound by any particular theory, the present disclosure notes that incorporation of one or more neutral internucleotidic linkages can be utilized to modulate melting temperature of duplexes formed between an oligonucleotide and its target nucleic acid.

Without wishing to be bound by any particular theory, the present disclosure notes that incorporation of one or more non-negatively charged internucleotidic linkages, e.g., neutral internucleotidic linkages, into an oligonucleotide may be able to increase the oligonucleotide's ability to mediate a function such as skipping of a deleterious exon in an USH2A gene transcript. In some embodiments, an oligonucleotide, e.g., an USH2A oligonucleotide capable of mediating an increase in the skipping of a deleterious exon in an USH2A gene transcript comprises one or more non-negatively charged internucleotidic linkages.

In some embodiments, a non-negatively charged internucleotidic linkage, e.g., a neutral internucleotidic linkage is not chirally controlled. In some embodiments, a non-negatively charged internucleotidic linkage is chirally controlled. In some embodiments, a non-negatively charged internucleotidic linkage is chirally controlled and its linkage phosphorus is Rp. In some embodiments, a non-negatively charged internucleotidic linkage is chirally controlled and its linkage phosphorus is Sp.

In some embodiments, an USH2A oligonucleotide comprises 1-20, 1-15, 1-10, 1-5, or 1, 2, 3, 4, 5, 6, 7, 8, 9, 10, or more non-negatively charged internucleotidic linkages. In some embodiments, an USH2A oligonucleotide comprises 1-20, 1-15, 1-10, 1-5, or 1, 2, 3, 4, 5, 6, 7, 8, 9, 10, or more neutral internucleotidic linkages. In some embodiments, each of non-negatively charged internucleotidic linkage and/or neutral internucleotidic linkages is optionally and independently chirally controlled. In some embodiments, each non-negatively charged internucleotidic linkage in an oligonucleotide is independently a chirally controlled internucleotidic linkage. In some embodiments, each neutral internucleotidic linkage in an oligonucleotide is independently a chirally controlled internucleotidic linkage. In some embodiments, at least one non-negatively charged internucleotidic linkage/neutral internucleotidic linkage has the structure of

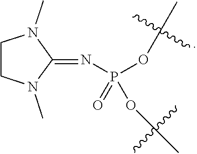 [SMILES: *OP(=O)(N=C1N(C)CCN1C)O*]

In some embodiments, an USH2A oligonucleotide comprises at least one non-negatively charged internucleotidic linkage wherein its linkage phosphorus is in Rp configuration, and at least one non-negatively charged internucleotidic linkage wherein its linkage phosphorus is in Sp configuration.

In many embodiments, as demonstrated extensively, oligonucleotides of the present disclosure comprise two or more different internucleotidic linkages. In some embodiments, an oligonucleotide comprises a phosphorothioate internucleotidic linkage and a non-negatively charged internucleotidic linkage. In some embodiments, an oligonucleotide comprises a phosphorothioate internucleotidic linkage, a non-negatively charged internucleotidic linkage, and a natural phosphate linkage. In some embodiments, a non-negatively charged internucleotidic linkage is a neutral internucleotidic linkage. In some embodiments, a non-negatively charged internucleotidic linkage is n001. In some embodiments, each phosphorothioate internucleotidic linkage is independently chirally controlled. In some embodiments, each chiral modified internucleotidic linkage is independently chirally controlled. In some embodiments, one or more non-negatively charged internucleotidic linkage are not chirally controlled.

A typical connection, as in natural DNA and RNA, is that an internucleotidic linkage forms bonds with two sugars (which can be either unmodified or modified as described herein). In many embodiments, as exemplified herein an internucleotidic linkage forms bonds through its oxygen atoms or heteroatoms with one optionally modified ribose or deoxyribose at its 5′ carbon, and the other optionally modified ribose or deoxyribose at its 3′ carbon. In some embodiments, each nucleoside units connected by an internucleotidic linkage independently comprises a nucleobase which is independently an optionally substituted A, T, C, G, or U, or an optionally substituted tautomer of A, T, C, G or U.

As appreciated by those skilled in the art, many other types of internucleotidic linkages may be utilized in accordance with the present disclosure, for example, those described in U.S. Pat. Nos. 3,687,808; 4,469,863; 4,476,301; 5,177,195; 5,023,243; 5,034,506; 5,166,315; 5,185,444; 5,188,897; 5,214,134; 5,216,141; 5,235,033; 5,264,423; 5,264,564; 5,276,019; 5,278,302; 5,286,717; 5,321,131; 5,399,676; 5,405,938; 5,405,939; 5,434,257; 5,453,496; 5,455,233; 5,466,677; 5,466,677; 5,470,967; 5,476,925; 5,489,677; 5,519,126; 5,536,821; 5,541,307; 5,541,316; 5,550,111; 5,561,225; 5,563,253; 5,571,799; 5,587,361; 5,596,086; 5,602,240; 5,608,046; 5,610,289; 5,618,704; 5,623,070; 5,625,050; 5,633,360; 5,64,562; 5,663,312; 5,677,437; 5,677,439; 6,160,109; 6,239,265; 6,028,188; 6,124,445; 6,169,170; 6,172,209; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; or RE39464. In some embodiments, a modified internucleotidic linkage is one described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612, the nucleobases, sugars, internucleotidic linkages, chiral auxiliaries/reagents, and technologies for oligonucleotide synthesis (reagents, conditions, cycles, etc.) of each of which is independently incorporated herein by reference.

In some embodiments, each internucleotidic linkage in an USH2A oligonucleotide is independently selected from a natural phosphate linkage, a phosphorothioate linkage, and a non-negatively charged internucleotidic linkage (e.g., n001). In some embodiments, each internucleotidic linkage in an USH2A oligonucleotide is independently selected from a natural phosphate linkage, a phosphorothioate linkage, and a neutral internucleotidic linkage (e.g., n001).

Various types of internucleotidic linkages may be utilized in combination of other structural elements, e.g., sugars, to achieve desired oligonucleotide properties and/or activities. For example, the present disclosure routinely utilizes modified internucleotidic linkages and modified sugars, optionally with natural phosphate linkages and natural sugars, in designing oligonucleotides. In some embodiments, the present disclosure provides an oligonucleotide comprising one or more modified sugars. In some embodiments, the present disclosure provides an oligonucleotide comprising one or more modified sugars and one or more modified internucleotidic linkages, one or more of which are natural phosphate linkages. In some embodiments, provided oligonucleotides comprise one or more 2′-F. In some embodiments, in provided oligonucleotides, a nucleoside comprising a 2′-modification is followed by a modified internucleotidic linkage. In some embodiments, in provided oligonucleotides, a nucleoside comprising a 2′-modification is preceded by a modified internucleotidic linkage. In some embodiments, a modified internucleotidic linkage is a chiral internucleotidic linkage. In some embodiments, a modified internucleotidic linkage is a phosphorothioate. In some embodiments, a chiral internucleotidic linkage is Sp. In some embodiments, in provided oligonucleotides, a nucleoside comprising a 2′-modification is followed by a Sp chiral internucleotidic linkage. In some embodiments, in provided oligonucleotides, a nucleoside comprising a 2′-F is followed by a Sp chiral internucleotidic linkage. In some embodiments, in provided oligonucleotides, a nucleoside comprising a 2′-modification is preceded by a Sp chiral internucleotidic linkage. In some embodiments, in provided oligonucleotides, a nucleoside comprising a 2′-F is preceded by a Sp chiral internucleotidic linkage. In some embodiments, a chiral internucleotidic linkage is Rp. In some embodiments, in provided oligonucleotides, a nucleoside comprising a 2′-modification is followed by a Rp chiral internucleotidic linkage. In some embodiments, in provided oligonucleotides, a nucleoside comprising a 2′-F is followed by a Rp chiral internucleotidic linkage. In some embodiments, in provided oligonucleotides, a nucleoside comprising a 2′-modification is preceded by a Rp chiral internucleotidic linkage. In some embodiments, in provided oligonucleotides, a nucleoside comprising a 2′-F is preceded by a Rp chiral internucleotidic linkage. In some embodiments, provided oligonucleotides are capable of directing an increase in the level of skipping of a deleterious exon in an USH2A gene transcript or a gene product thereof. In some embodiments, oligonucleotides of a plurality comprise one or more natural phosphate linkages and one or more modified internucleotidic linkages.

### Oligonucleotide Compositions

Among other things, the present disclosure provides various oligonucleotide compositions. In some embodiments, the present disclosure provides oligonucleotide compositions of oligonucleotides described herein. In some embodiments, an oligonucleotide composition, e.g., an USH2A oligonucleotide composition, comprises a plurality of an oligonucleotide described in the present disclosure. In some embodiments, an oligonucleotide composition, e.g., an USH2A oligonucleotide composition, is chirally controlled. In some embodiments, an oligonucleotide composition, e.g., an USH2A oligonucleotide composition, is not chirally controlled (stereorandom).

Linkage phosphorus of natural phosphate linkages is achiral. Linkage phosphorus of many modified internucleotidic linkages, e.g., phosphorothioate internucleotidic linkages, are chiral. In some embodiments, during preparation of oligonucleotide compositions (e.g., in traditional phosphoramidite oligonucleotide synthesis), configurations of chiral linkage phosphorus are not purposefully designed or controlled, creating non-chirally controlled (stereorandom) oligonucleotide compositions (substantially racemic preparations) which are complex, random mixtures of various stereoisomers (diastereoisomers)—for oligonucleotides with n chiral internucleotidic linkages (linkage phosphorus being chiral), typically 2ⁿ stereoisomers (e.g., when n is 10, 2¹⁰=1,032; when n is 20, 2²⁰=1,048,576). These stereoisomers have the same constitution, but differ with respect to the pattern of stereochemistry of their linkage phosphorus.

In some embodiments, stereorandom oligonucleotide compositions have sufficient properties and/or activities for certain purposes and/or applications. In some embodiments, stereorandom oligonucleotide compositions can be cheaper, easier and/or simpler to produce than chirally controlled oligonucleotide compositions. However, stereoisomers within stereorandom compositions may have different properties, activities, and/or toxicities, resulting in inconsistent therapeutic effects and/or unintended side effects by stereorandom compositions, particularly compared to certain chirally controlled oligonucleotide compositions of oligonucleotides of the same constitution.

In some embodiments, the present disclosure encompasses technologies for designing and preparing chirally controlled oligonucleotide compositions. In some embodiments, the present disclosure provides chirally controlled oligonucleotide compositions, e.g., of many oligonucleotides in Table A1 which contain S and/or R in their stereochemistry/linkage. In some embodiments, a chirally controlled oligonucleotide composition comprises a controlled/pre-determined (not random as in stereorandom compositions) level of a plurality of oligonucleotides, wherein the oligonucleotides share the same linkage phosphorus stereochemistry at one or more chiral internucleotidic linkages (chirally controlled internucleotidic linkages). In some embodiments, the oligonucleotides share the same pattern of backbone chiral centers (stereochemistry of linkage phosphorus). In some embodiments, a pattern of backbone chiral centers is as described in the present disclosure. In some embodiments, the oligonucleotides are structural identical.

In some embodiments, an oligonucleotide composition is a chirally controlled oligonucleotide composition comprising a plurality of oligonucleotides, wherein the oligonucleotides share:
1) a common base sequence,
2) a common pattern of backbone linkages, and
3) the same linkage phosphorus stereochemistry at one or more (e.g., 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, or more) chiral internucleotidic linkages (chirally controlled internucleotidic linkages),
wherein the composition is enriched, relative to a substantially racemic preparation of oligonucleotides sharing the common base sequence and pattern of backbone linkages, for oligonucleotides of the plurality.

In some embodiments, an oligonucleotide composition is a chirally controlled oligonucleotide composition comprising a plurality of oligonucleotides, wherein the oligonucleotides share:
1) a common constitution, and
2) the same linkage phosphorus stereochemistry at one or more (e.g., 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, or more) chiral internucleotidic linkages (chirally controlled internucleotidic linkages),
wherein the composition is enriched, relative to a substantially racemic preparation of oligonucleotides sharing the common constitution for oligonucleotides of the plurality.

In some embodiments, an oligonucleotide composition is a chirally controlled oligonucleotide composition comprising a plurality of oligonucleotides, wherein the oligonucleotides share:
1) a common base sequence,
2) a common patter of backbone linkages, and
3) a common pattern of backbone chiral centers, which pattern comprises at least one Sp,
wherein the composition is enriched, relative to a substantially racemic preparation of oligonucleotides sharing the common base sequence and pattern of backbone linkages, for oligonucleotides of the plurality.

In some embodiments, an oligonucleotide composition is a chirally controlled oligonucleotide composition comprising a plurality of oligonucleotides, wherein the oligonucleotides share:
1) a common base sequence,
2) a common patter of backbone linkages, and
3) a common pattern of backbone chiral centers, which pattern comprises at least one Rp,
wherein the composition is enriched, relative to a substantially racemic preparation of oligonucleotides sharing the common base sequence and pattern of backbone linkages, for oligonucleotides of the plurality.

In some embodiments, oligonucleotides of a plurality share the same linkage phosphorus stereochemistry at one or more (e.g., 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, or more) chiral internucleotidic linkages. In some embodiments, oligonucleotides of a plurality share the same linkage phosphorus stereochemistry at five or more (e.g., 5-50, 5-40, 5-30, 5-25, 5-20, 5-15, 5-10, 5, 6, 7, 8, 9, 10, 11, 12, 13, 14, 15, 16, 17, 18, 19, 20, or more) chiral internucleotidic linkages. In some embodiments, each chiral internucleotidic linkage is independently chirally controlled. In some embodiments, an enrichment relative to a racemic preparation is that about 1-100% of all oligonucleotides within the composition that share the common base sequence and pattern of backbone linkages are oligonucleotides of the plurality. In some embodiments, an enrichment relative to a racemic preparation is that about 1-100% of all oligonucleotides within the composition that share the common constitution are oligonucleotides of the plurality. In some embodiments, the present disclosure provides an oligonucleotide composition comprising an oligonucleotide, wherein about 1-100% of all oligonucleotides within the composition that share the same base sequence as the oligonucleotide share the same pattern of backbone chiral centers as the oligonucleotide. In some embodiments, the present disclosure provides an oligonucleotide composition comprising an oligonucleotide, wherein about 1-100% of all oligonucleotides within the composition that share the same base sequence as the oligonucleotide share the same oligonucleotide chain as the oligonucleotide. In some embodiments, the present disclosure provides an oligonucleotide composition comprising an oligonucleotide, wherein about 1-100% of all oligonucleotides within the composition that share the same base sequence as the oligonucleotide have the structure of the oligonucleotide, or an acid, base, or salt form thereof. In some embodiments, a composition is a liquid composition, and oligonucleotides are dissolved in a solution. In some embodiments, a percentage is about, or is at least about 5%, 10%, 15%, 20%, 25%, 30%, 35%, 40%, 45%, 50%, 60%, 70%, 75%, 80%, 85%, 90%, 95%, 96%, 97%, 98%, or 99%. In some embodiments, a percentage is about, or is at least about 50%. In some embodiments, a percentage is about, or is at least about 60%. In some embodiments, a percentage is about, or is at least about 70%. In some embodiments, a percentage is about, or is at least about 75%. In some embodiments, a percentage is about, or is at least about 80%. In some embodiments, a percentage is about, or is at least about 85%. In some embodiments, a percentage is about, or is at least about 90%. In some embodiments, a percentage is about, or is at least about 95%. In some embodiments, a percentage is about, or is at least about 97%. In some embodiments, a percentage is about, or is at least about 98%. In some embodiments, a percentage is about, or is at least about 99%. As appreciated by those skilled in the art, various forms of an oligonucleotide may be properly considered to have the same constitution and/or structure, and various forms of oligonucleotides sharing the same constitution may be properly considered to have the same constitution.

In some embodiments, oligonucleotides of a plurality are of the same constitution. In some embodiments, the present disclosure provides a chirally controlled oligonucleotide composition comprising a plurality of oligonucleotides, wherein the oligonucleotides are of a common constitution, and share the same linkage phosphorus stereochemistry at one or more (e.g., 1-50, 1-40, 1-30, 1-25, 1-20, 1, 2, 3, 4, 5, 6, 7, 8, 9, 10, 11, 12, 13, 14, 15, 16, 17, 18, 19, 20, 21, 22, 23, 24, 25 or more) chiral internucleotidic linkages (chirally controlled internucleotidic linkages), wherein the composition is enriched, relative to a substantially racemic preparation of oligonucleotides of the common constitution, for oligonucleotides of the plurality.

In some embodiments, oligonucleotides of a plurality are structurally identical. In some embodiments, the present disclosure provides a chirally controlled oligonucleotide composition comprising a plurality of oligonucleotides, wherein the oligonucleotides are structurally identical, and share the same linkage phosphorus stereochemistry at one or more (e.g., 1-50, 1-40, 1-30, 1-25, 1-20, 1, 2, 3, 4, 5, 6, 7, 8, 9, 10, 11, 12, 13, 14, 15, 16, 17, 18, 19, 20, 21, 22, 23, 24, 25 or more) chiral internucleotidic linkages (chirally controlled internucleotidic linkages), wherein the composition is enriched, relative to a substantially racemic preparation of oligonucleotides of the same constitution as the oligonucleotides of the plurality, for oligonucleotides of the plurality.

In some embodiments, an enrichment relative to a substantially racemic preparation is that at least about 5%, 10%, 20%, 30%, 40%, 50%, 60%, 70%, 80%, 85%, 90%, 91%, 92%, 93%, 94%, 95%, 96%, 97%, 98%, or 99% of all oligonucleotides in the composition are oligonucleotide of the plurality. In some embodiments, the percentage is at least about 10%. In some embodiments, the percentage is at least about 20%. In some embodiments, the percentage is at least about 30%. In some embodiments, the percentage is at least about 40%. In some embodiments, the percentage is at least about 50%. In some embodiments, the percentage is at least about 60%. In some embodiments, the percentage is at least about 70%. In some embodiments, the percentage is at least about 75%. In some embodiments, the percentage is at least about 80%. In some embodiments, the percentage is at least about 85%. In some embodiments, the percentage is at least about 90%. In some embodiments, the percentage is at least about 91%. In some embodiments, the percentage is at least about 92%. In some embodiments, the percentage is at least about 93%. In some embodiments, the percentage is at least about 94%. In some embodiments, the percentage is at least about 95%. In some embodiments, the percentage is at least about 96%. In some embodiments, the percentage is at least about 97%. In some embodiments, the percentage is at least about 98%. In some embodiments, the percentage is at least about 99%.

Levels of oligonucleotides of a plurality in chirally controlled oligonucleotide compositions are controlled. In contrast, in non-chirally controlled (or stereorandom, racemic) oligonucleotide compositions (or preparations), levels of oligonucleotides are random and not controlled. In some embodiments, a level of the oligonucleotides of a plurality in a chirally controlled oligonucleotide composition is about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, or 99%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in the chirally controlled oligonucleotide composition, or of all oligonucleotides in the chirally controlled oligonucleotide composition that share the common base sequence as the oligonucleotides of the plurality, or of all oligonucleotides in the chirally controlled oligonucleotide composition that share the common base sequence and pattern of backbone linkages as the oligonucleotides of the plurality, or of all oligonucleotides in the chirally controlled oligonucleotide composition that share the common base sequence, pattern of backbone linkages as and pattern of backbone phosphorus modifications as the oligonucleotides of the plurality, or of all oligonucleotides in the chirally controlled oligonucleotide composition that share the same constitution as oligonucleotides of the plurality. In some embodiments, an enrichment relative to a substantially racemic preparation is a level described herein.

In some embodiments, a level as a percentage (e.g., a controlled level, a pre-determined level, an enrichment) is or is at least (DS)^{nc}, wherein DS is 90%-100%, and nc is the number of chirally controlled internucleotidic linkages as described in the present disclosure (e.g., 5, 6, 7, 8, 9, 10, 11, 12, 13, 14, 15, 16, 17, 18, 19, 20 or more). In some embodiments, each chiral internucleotidic linkage is chirally controlled, and nc is the number of chiral internucleotidic linkage. In some embodiments, DS is 90%, 91%, 92%, 93%, 94%, 95%, 96%, 97%, 98%, 99% or 99.5% or more. In some embodiments, DS is or is at least 90%. In some embodiments, DS is or is at least 91%. In some embodiments, DS is or is at least 92%. In some embodiments, DS is or is at least 93%. In some embodiments, DS is or is at least 94%. In some embodiments, DS is or is at least 95%. In some embodiments, DS is or is at least 96%. In some embodiments, DS is or is at least 97%. In some embodiments, DS is or is at least 98%. In some embodiments, DS is or is at least 99%. In some embodiments, a level (e.g., a controlled level, a pre-determined level, an enrichment) is a percentage of all oligonucleotides in a composition that share the same constitution, wherein the percentage is or is at least (DS)^{nc}. For example, when DS is 99% and nc is 10, the percentage is or is at least 90% ((99%)¹⁰≈0.90=90%). As appreciated by those skilled in the art, in a stereorandom preparation the percentage is typically about ½^{nc}—when nc is 10, the percentage is about ½¹⁰≈0.001=0.1%.

In some embodiments, an oligonucleotide composition is a chirally controlled oligonucleotide composition comprising a plurality of oligonucleotides, wherein the oligonucleotides share:
1) a common base sequence,
2) a common pattern of backbone linkages, and
3) the same linkage phosphorus stereochemistry at one or more (e.g., 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, or more) chiral internucleotidic linkages (chirally controlled internucleotidic linkages),
wherein the percentage of the oligonucleotides of the plurality within all oligonucleotides in the composition that share the common base sequence and pattern of backbone linkages is at least (DS)^{nc}, wherein DS is 90%-100%, and nc is the number of chirally controlled internucleotidic linkages.

In some embodiments, an oligonucleotide composition is a chirally controlled oligonucleotide composition comprising a plurality of oligonucleotides, wherein the oligonucleotides share:
1) a common constitution, and
2) the same linkage phosphorus stereochemistry at one or more (e.g., 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, or more) chiral internucleotidic linkages (chirally controlled internucleotidic linkages),
wherein the percentage of the oligonucleotides of the plurality within all oligonucleotides in the composition that share the common constitution is at least (DS)^{nc}, wherein DS is 90%-100%, and nc is the number of chirally controlled internucleotidic linkages.

In some embodiments, an oligonucleotide composition is a chirally controlled oligonucleotide composition comprising a plurality of oligonucleotides, wherein the oligonucleotides share:
1) a common base sequence,
2) a common patter of backbone linkages, and
3) a common pattern of backbone chiral centers, which pattern comprises at least one Sp,
wherein the percentage of the oligonucleotides of the plurality within all oligonucleotides in the composition that share the common base sequence and pattern of backbone linkages is at least (DS)^{nc}, wherein DS is 90%-100%, and nc is the number of chirally controlled internucleotidic linkages.

In some embodiments, an oligonucleotide composition is a chirally controlled oligonucleotide composition comprising a plurality of oligonucleotides, wherein the oligonucleotides share:
1) a common base sequence,
2) a common patter of backbone linkages, and
3) a common pattern of backbone chiral centers, which pattern comprises at least one Rp,
wherein the percentage of the oligonucleotides of the plurality within all oligonucleotides in the composition that share the common base sequence and pattern of backbone linkages is at least (DS)^{nc}, wherein DS is 90%-100%, and nc is the number of chirally controlled internucleotidic linkages.

In some embodiments, the present disclosure provides a chirally controlled oligonucleotide composition comprising a plurality of oligonucleotides, wherein the oligonucleotides are of a common constitution, and share the same linkage phosphorus stereochemistry at one or more (e.g., 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, or more) chiral internucleotidic linkages (chirally controlled internucleotidic linkages), wherein the percentage of the oligonucleotides of the plurality within all oligonucleotides of the same constitution in the composition is at least (DS)^{nc}, wherein DS is 90%-100%, and nc is the number of chirally controlled internucleotidic linkages.

In some embodiments, the present disclosure provides a chirally controlled oligonucleotide composition comprising a plurality of oligonucleotides, wherein the oligonucleotides are structurally identical, and share the same linkage phosphorus stereochemistry at one or more (e.g., 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, or more) chiral internucleotidic linkages (chirally controlled internucleotidic linkages), wherein the percentage of the oligonucleotides of the plurality within all oligonucleotides of the same constitution as the oligonucleotides of the plurality in the composition is at least (DS)^{nc}, wherein DS is 90%-100%, and nc is the number of chirally controlled internucleotidic linkages.

In some embodiments, oligonucleotides of the plurality are of different salt forms. In some embodiments, oligonucleotides of the plurality comprise one or more forms, e.g., various pharmaceutically acceptable salt forms, of a single oligonucleotide. In some embodiments, oligonucleotides of the plurality comprise one or more forms, e.g., various pharmaceutically acceptable salt forms, of two or more oligonucleotides. In some embodiments, oligonucleotides of the plurality comprise one or more forms, e.g., various pharmaceutically acceptable salt forms, of 2^{NCC} oligonucleotides, wherein NCC is the number of non-chirally controlled chiral internucleotidic linkages. In some embodiments, the 2^{NCC} oligonucleotides have relatively similar levels within a composition as, e.g., none of them are specifically enriched using chirally controlled oligonucleotide synthesis.

In some embodiments, level of a plurality of oligonucleotides in a composition can be determined as the product of the diastereopurity of each chirally controlled internucleotidic linkage in the oligonucleotides. In some embodiments, diastereopurity of an internucleotidic linkage connecting two nucleosides in an oligonucleotide (or nucleic acid) is represented by the diastereopurity of an internucleotidic linkage of a dimer connecting the same two nucleosides, wherein the dimer is prepared using comparable conditions, in some instances, identical synthetic cycle conditions (e.g., for the linkage between Nx and Ny in an oligonucleotide . . . NxNy . . . , the dimer is NxNy).

In some embodiments, all chiral internucleotidic linkages are chiral controlled, and the composition is a completely chirally controlled oligonucleotide composition. In some embodiments, not all chiral internucleotidic linkages are chiral controlled internucleotidic linkages, and the composition is a partially chirally controlled oligonucleotide composition. In some embodiments, at least 10%, 20%, 30%, 40%, 50%, 60%, 70%, 80%, 85%, 90%, 91%, 92%, 93%, 94%, 95%, 96%, 97%, 98%, or 99% of all chiral internucleotidic linkages are chirally controlled. In some embodiments, at least 50%, 60%, 70%, 80%, 85%, 90%, 91%, 92%, 93%, 94%, 95%, 96%, 97%, 98%, or 99% of all chiral internucleotidic linkages are chirally controlled.

Oligonucleotides may comprise or consist of various patterns of backbone chiral centers (patterns of stereochemistry of chiral linkage phosphorus). Certain useful patterns of backbone chiral centers are described in the present disclosure. In some embodiments, a plurality of oligonucleotides share a common pattern of backbone chiral centers, which is or comprises a pattern described in the present disclosure (e.g., as in “Linkage Phosphorus Stereochemistry and Patterns Thereof”, a pattern of backbone chiral centers of a chirally controlled oligonucleotide in Table A1, etc.).

In some embodiments, a chirally controlled oligonucleotide composition is chirally pure (or stereopure, stereochemically pure) oligonucleotide composition, wherein the oligonucleotide composition comprises a plurality of oligonucleotides, wherein the oligonucleotides are identical [including that each chiral element of the oligonucleotides, including each chiral linkage phosphorus, is independently defined (stereodefined)], and the composition does not contain other stereoisomers. A chirally pure (or stereopure, stereochemically pure) oligonucleotide composition of an oligonucleotide stereoisomer does not contain other stereoisomers (as appreciated by those skilled in the art, one or more unintended stereoisomers may exist as impurities—example purities are descried in the present disclosure).

Chirally controlled oligonucleotide compositions can demonstrate a number of advantages over stereorandom oligonucleotide compositions. Among other things, chirally controlled oligonucleotide compositions are more uniform than corresponding stereorandom oligonucleotide compositions with respect to oligonucleotide structures. By controlling stereochemistry, compositions of individual stereoisomers can be prepared and assessed, so that chirally controlled oligonucleotide composition of stereoisomers with desired properties and/or activities can be developed. In some embodiments, chirally controlled oligonucleotide compositions provides better delivery, stability, clearance, activity, selectivity, and/or toxicity profiles compared to, e.g., corresponding stereorandom oligonucleotide compositions. In some embodiments, chirally controlled oligonucleotide compositions provide better efficacy, fewer side effects, and/or more convenient and effective dosage regimens. Among other things, patterns of backbone chiral centers as described herein can be utilized to provide controlled cleavage of oligonucleotide targets (e.g., transcripts such as pre-mRNA, mature mRNA, etc; including control of cleavage sites, rate and/or extent of cleavage at cleavage sites, and/or overall rate and extent of cleavage, etc.) and greatly increased target selectivity. In some embodiments, chirally controlled oligonucleotide compositions of oligonucleotides comprising certain patterns of backbone chiral centers can differentiate sequences with nucleobase difference at very few positions, in some embodiments, at single position (e.g., at SNP site, point mutation site, etc.).

In some embodiments, the present disclosure provides a stereorandom oligonucleotide composition, e.g., a stereorandom USH2A oligonucleotide composition. In some embodiments, the present disclosure provides a stereorandom USH2A oligonucleotide composition which is capable of increasing the level of skipping of a deleterious exon in a mutant USH2A gene transcript or a gene product thereof. In some embodiments, the present disclosure provides a stereorandom USH2A oligonucleotide composition which is capable of increasing the level of skipping of a deleterious exon in a mutant USH2A gene transcript or a gene product thereof, and wherein the base sequence of the USH2A oligonucleotides is, comprises, or comprises a span (e.g., at least 10 or 15 contiguous bases) of a base sequence disclosed herein (e.g., a base sequence in Table A1, wherein each U may be independently replaced with T and vice versa). In some embodiments, the present disclosure provides a stereorandom USH2A oligonucleotide composition which is capable of increasing the level of skipping of a deleterious exon in a mutant USH2A gene transcript or a gene product thereof, and wherein the base sequence of the USH2A oligonucleotides is or comprises a base sequence disclosed herein (e.g., a base sequence in Table A1, wherein each U may be independently replaced with T and vice versa). In some embodiments, the present disclosure provides a stereorandom USH2A oligonucleotide composition which is capable of increasing the level of skipping of a deleterious exon in a mutant USH2A gene transcript or a gene product thereof, and wherein the base sequence of the USH2A oligonucleotides is a base sequence disclosed herein (e.g., a base sequence in Table A1, wherein each U may be independently replaced with T and vice versa).

Non-limiting examples of stereopure (or chirally controlled) oligonucleotide compositions, e.g., stereopure (or chirally controlled) USH2A oligonucleotide compositions, are described herein, including but not limited to: WV-20891, WV-20892, WV-20902, WV-20908, WV-20988, WV-21008, WV-24297, WV-24368, WV-24376, WV-24366, WV-24375, WV-24360, WV-24298, WV-24381, WV-24382, WV-21100, WV-21105, and WV-20885.

In some embodiments, the present disclosure pertains to: A chirally controlled composition comprising an USH2A oligonucleotide capable of mediating the skipping of at least one exon of USH2A.

In some embodiments, the present disclosure pertains to: A chirally controlled composition comprising an USH2A oligonucleotide capable of mediating the skipping of at least one exon of USH2A, wherein the oligonucleotide comprises at least one chirally controlled phosphorothioate.

In some embodiments, the present disclosure pertains to: A chirally controlled composition comprising an USH2A oligonucleotide capable of mediating the skipping of at least one exon of USH2A, wherein the oligonucleotide comprises at least one chirally controlled phosphorothioate and at least one neutral or non-negatively charged internucleotidic linkage.

In some embodiments, the present disclosure pertains to: A chirally controlled composition comprising an USH2A oligonucleotide capable of mediating the skipping of at least one exon of USH2A, wherein the exon is exon 13.

In some embodiments, the present disclosure pertains to: A chirally controlled composition comprising an USH2A oligonucleotide capable of mediating the skipping of at least one exon of USH2A, wherein the exon is exon 13, and the oligonucleotide comprises at least one chirally controlled phosphorothioate.

In some embodiments, the present disclosure pertains to: A chirally controlled composition comprising an USH2A oligonucleotide capable of mediating the skipping of at least one exon of USH2A, wherein the exon is exon 13, and the oligonucleotide comprises at least one chirally controlled phosphorothioate and at least one neutral or non-negatively charged internucleotidic linkage.

In some embodiments, an oligonucleotide composition comprises one or more internucleotidic linkages which are stereocontrolled (chirally controlled; in some embodiments, stereopure) and one or more internucleotidic linkages which are stereorandom. In some embodiments, an USH2A oligonucleotide composition comprises one or more internucleotidic linkages which are stereocontrolled (chirally controlled; in some embodiments, stereopure) and one or more internucleotidic linkages which are stereorandom.

In some embodiments, an oligonucleotide composition comprises one or more internucleotidic linkages which are stereocontrolled (e.g., chirally controlled or stereopure) and one or more internucleotidic linkages which are stereorandom. Such oligonucleotides may target various targets and may have various base sequences, and may be capable of operating via one or more of various modalities (e.g., RNase H mechanism, steric hindrance, double- or single-stranded RNA interference, exon skipping modulation, CRISPR, aptamer, etc.).

As understood by a person having ordinary skill in the art, stereorandom or (substantially) racemic preparations/non-chirally controlled oligonucleotide compositions are typically prepared without using chiral auxiliaries, chiral modification reagents, and/or chiral catalysts that can provide high stereoselectivity (e.g., 90%, 91%, 92%, 93%, 94%, 95%, 96%, 97%, 98%, 99% or 99.5% or more; in some embodiments, 95%, 96%, 97%, 98%, 99% or 99.5% or more; in some embodiments, 97%, 98%, 99% or 99.5% or more; in some embodiments, 98%, 99% or 99.5% or more) at linkage phosphorus during oligonucleotide synthesis. In some embodiments, in a substantially racemic (or chirally uncontrolled) preparation of oligonucleotides, coupling steps are not chirally controlled in that the coupling steps are not specifically conducted to provide enhanced stereoselectivity. An example substantially racemic preparation of oligonucleotides/non-chirally controlled oligonucleotide composition is a preparation of phosphorothioate oligonucleotides through traditional phosphoramidite oligonucleotide synthesis and sulfurization with non-chiral sulfurization reagents such as tetraethylthiuram disulfide or (TETD), 3H-1, 2-bensodithiol-3-one 1, 1-dioxide (BDTD), etc., which are well-known processes. Various methods for making stereorandom oligonucleotide compositions/substantially racemic preparations of oligonucleotides are widely known and practiced in the art and can be utilized for preparing such compositions and preparations of the present disclosure.

In some embodiments, the present disclosure provides a chirally controlled oligonucleotide composition, e.g., chirally controlled USH2A oligonucleotide composition. In some embodiments, provided chirally controlled oligonucleotide compositions comprise a plurality of oligonucleotides, e.g., USH2A oligonucleotides, of the same constitution, and have one or more (e.g., 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, or more) internucleotidic linkages. In some embodiments, a plurality of oligonucleotides, e.g., in a chirally controlled oligonucleotide composition, is a plurality of an oligonucleotide selected from Table A1, wherein the oligonucleotide comprises at least one Rp or Sp linkage phosphorus in a chirally controlled internucleotidic linkage. In some embodiments, a plurality of oligonucleotides, e.g., in a chirally controlled oligonucleotide composition, is a plurality of an oligonucleotide selected from Table A1, wherein each phosphorothioate internucleotidic linkage in the oligonucleotide is independently chirally controlled (each phosphorothioate internucleotidic linkage is independently Rp or Sp). In some embodiments, an oligonucleotide composition, e.g., an USH2A oligonucleotide composition is a substantially pure preparation of a single oligonucleotide in that oligonucleotides in the composition that are not the single oligonucleotide are impurities from the preparation process of the single oligonucleotide, in some case, after certain purification procedures. In some embodiments, a single oligonucleotide is an oligonucleotide of Table A1, wherein each chiral internucleotidic linkage of the oligonucleotide is chirally controlled (e.g., indicated as S or R but not X in “Stereochemistry/Linkage”).

In some embodiments, a chirally controlled oligonucleotide composition can have, relative to a corresponding stereorandom oligonucleotide composition, increased activity and/or stability, increased delivery, and/or decreased ability to elicit adverse effects such as complement, TLR9 activation, etc. In some embodiments, a stereorandom (non-chirally controlled) oligonucleotide composition differs from a chirally controlled oligonucleotide composition in that its corresponding plurality of oligonucleotides do not contain any chirally controlled internucleotidic linkages but the stereorandom oligonucleotide composition is otherwise identical to the chirally controlled oligonucleotide composition.

In some embodiments, the present disclosure pertains to a chirally controlled USH2A oligonucleotide composition which is capable of increasing the level of skipping of a deleterious exon in a mutant USH2A gene transcript or a gene product thereof.

In some embodiments, the present disclosure provides a chirally controlled USH2A oligonucleotide composition which is capable of increasing the level of skipping of a deleterious exon in a mutant USH2A gene transcript or a gene product thereof, and comprises a plurality of oligonucleotides which share a common base sequence that is, comprises, or comprises a span (e.g., at least 10 or 15 contiguous bases) of a base sequence disclosed herein (e.g., in Table A1, wherein each U may be independently replaced with T and vice versa). In some embodiments, the present disclosure provides a chirally controlled USH2A oligonucleotide composition which is capable of increasing the level of skipping of a deleterious exon in a mutant USH2A gene transcript or a gene product thereof, and comprises a plurality of oligonucleotides which share a common base sequence that is or comprises a base sequence disclosed herein (e.g., in Table A1, wherein each U may be independently replaced with T and vice versa). In some embodiments, the present disclosure provides a chirally controlled USH2A oligonucleotide composition which is capable of increasing the level of skipping of a deleterious exon in a mutant USH2A gene transcript or a gene product thereof, and comprises a plurality of oligonucleotides which share a common base sequence that is a base sequence disclosed herein (e.g., in Table A1, wherein each U may be independently replaced with T and vice versa).

In some embodiments, a provided chirally controlled oligonucleotide composition is a chirally controlled USH2A oligonucleotide composition comprising a plurality of USH2A oligonucleotides. In some embodiments, a chirally controlled oligonucleotide composition is a chirally pure (or “stereochemically pure”) oligonucleotide composition. In some embodiments, the present disclosure provides a chirally pure oligonucleotide composition of an oligonucleotide in Table A1, wherein each chiral internucleotidic linkage of the oligonucleotide is independently chirally controlled (Rp or Sp, e.g., can be determined from R or S but not X in “Stereochemistry/Linkage”). As appreciated by those skilled in the art, in a chirally controlled or chirally pure composition of an oligonucleotide, the percentage of the oligonucleotide in the composition is significantly higher [e.g., 10, 11, 12, 13, 14, 15, 16, 17, 18, 19, 20, 21, 22, 23, 24, 25, 30, 35, 40, 45, 50, 60, 70, 80, 90, 100, 10³, 10⁴, 10⁵ or more, or 10^{nc}, 15^{nc}, 20^{nc}, 25^{nc}, 30^{nc}, 35^{nc}, 40^{nc}, 45^{nc}, 50^{nc}, 60^{nc}, 70^{nc}, 80^{nc}, 90^{nc}, 100^{nc} or more, fold of the percentage of another stereoisomer, wherein nc is the number of chirally controlled internucleotidic linkage(s)] than any other possible stereoisomers, which may exist in the composition as impurities. As one of ordinary skill in the art will understand, chemical selectivity rarely, if ever, achieves completeness (absolute 100%). In some embodiments, a chirally pure oligonucleotide composition comprises a plurality of oligonucleotides, wherein oligonucleotides of the plurality are structurally identical and all have the same structure (the same stereoisomeric form; in the context of oligonucleotide, typically the same diastereomeric form as typically multiple chiral centers exist in an oligonucleotide), and the chirally pure oligonucleotide composition does not contain any other stereoisomers (in the context of oligonucleotide, typically diastereomers as typically multiple chiral centers exist in an oligonucleotide; to the extent, e.g., achievable by stereoselective preparation). As appreciated by those skilled in the art, stereorandom (or “racemic”, “non-chirally controlled”) oligonucleotide compositions are random mixtures of many stereoisomers (e.g., 2ⁿ diastereoisomers wherein n is the number of chiral linkage phosphorus for oligonucleotides in which other chiral centers (e.g., carbon chiral centers in sugars) are chirally controlled each independently existing in one configuration and only chiral linkage phosphorus centers are not chirally controlled).

Certain data showing properties and/or activities of chirally controlled oligonucleotide composition, e.g., chirally controlled USH2A oligonucleotide compositions in increasing the level of skipping of a deleterious exon in a mutant USH2A gene transcript or a gene product thereof, are shown in, for example, the Examples section of this document.

In some embodiments, the present disclosure provides an oligonucleotide composition comprising oligonucleotides that comprise at least one chiral linkage phosphorus. In some embodiments, the present disclosure provides an USH2A oligonucleotide composition comprising USH2A oligonucleotides that comprise at least one chiral linkage phosphorus. In some embodiments, the present disclosure provides an USH2A oligonucleotide composition in which the USH2A oligonucleotides comprise a chirally controlled phosphorothioate internucleotidic linkage, wherein the linkage phosphorus has a Rp configuration. In some embodiments, the present disclosure provides an USH2A oligonucleotide composition in which the USH2A oligonucleotides comprise a chirally controlled phosphorothioate internucleotidic linkage, wherein the linkage phosphorus has a Sp configuration.

In some embodiments, compared to reference oligonucleotide compositions, provided chirally controlled oligonucleotide compositions (e.g., chirally controlled USH2A oligonucleotide compositions) are surprisingly effective. In some embodiments, desired biological effects (e.g., as measured by decreased levels of mRNA, proteins, etc. whose levels are targeted for reduction) can be enhanced by more than 5, 10, 15, 20, 25, 30, 40, 50, or 100 fold (e.g., as measured by remaining levels of mRNA, proteins, etc.). In some embodiments, a change is measured by decrease of an undesired mRNA level compared to a reference condition. In some embodiments, a change is measured by increase of a desired mRNA level compared to a reference condition. In some embodiments, a change is measured by decrease of an undesired mRNA level compared to a reference condition. In some embodiments, a reference condition is absence of treatment, e.g., by a chirally controlled oligonucleotide composition. In some embodiments, a reference condition is a corresponding stereorandom composition of oligonucleotides having the same constitution.

In some embodiments, the present disclosure provides a chirally controlled oligonucleotide composition, e.g., a chirally controlled USH2A oligonucleotide composition, wherein the linkage phosphorus of at least one chirally controlled internucleotidic linkage is Sp. In some embodiments, the present disclosure provides a chirally controlled oligonucleotide composition, e.g., a chirally controlled USH2A oligonucleotide composition, wherein the majority of linkage phosphorus of chirally controlled internucleotidic linkages are Sp. In some embodiments, about 50%-100%, 55%-100%, 60%-100%, 65%-100%, 70%-100%, 75%-100%, 80%-100%, 85%-100%, 90%-100%, 55%-95%, 60%-95%, 65%-95%, or about 55%, 60%, 65%, 70%, 75%, 80%, 85%, 90%, 95%, 97%, 99% or more, of all chirally controlled internucleotidic linkages (or of all chiral internucleotidic linkages, or of all internucleotidic linkages) are Sp. In some embodiments, the present disclosure provides a chirally controlled oligonucleotide composition, e.g., a chirally controlled USH2A oligonucleotide composition, wherein the majority of chiral internucleotidic linkages are chirally controlled and are Sp at their linkage phosphorus. In some embodiments, the present disclosure provides a chirally controlled oligonucleotide composition, e.g., a chirally controlled USH2A oligonucleotide composition, wherein each chiral internucleotidic linkage is chirally controlled and each chiral linkage phosphorus is Sp. In some embodiments, the present disclosure provides a chirally controlled oligonucleotide composition, e.g., chirally controlled USH2A oligonucleotide composition, wherein at least one chirally controlled internucleotidic linkage has a Rp linkage phosphorus. In some embodiments, the present disclosure provides a chirally controlled oligonucleotide composition, e.g., a chirally controlled USH2A oligonucleotide composition, wherein at least one chirally controlled internucleotidic linkage comprises a Rp linkage phosphorus and at least one chirally controlled internucleotidic linkage comprises a Sp linkage phosphorus.

In some embodiments, the present disclosure provides a chirally controlled oligonucleotide composition, wherein at least two chirally controlled internucleotidic linkages have different linkage phosphorus stereochemistry and/or different P-modifications relative to one another, wherein a P-modification is a modification at a linkage phosphorus. In some embodiments, the present disclosure provides a chirally controlled oligonucleotide composition, wherein at least two chirally controlled internucleotidic linkages have different stereochemistry relative to one another, and the pattern of the backbone chiral centers of the oligonucleotides is characterized by a repeating pattern of alternating stereochemisty.

In certain embodiments, the present disclosure provides a chirally controlled oligonucleotide composition comprising a plurality of oligonucleotides, wherein with in each of the oligonucleotides at least two individual internucleotidic linkages have different P-modifications relative to one another. In certain embodiments, the present disclosure provides a chirally controlled oligonucleotide composition comprising a plurality of oligonucleotides, wherein with in each of the oligonucleotides at least two individual internucleotidic linkages have different P-modifications relative to one another, and each of the oligonucleotide comprises a natural phosphate linkage. In certain embodiments, the present disclosure provides a chirally controlled oligonucleotide composition comprising a plurality of oligonucleotides, wherein with in each of the oligonucleotides at least two individual internucleotidic linkages have different P-modifications relative to one another, and each of the oligonucleotide comprises a phosphorothioate internucleotidic linkage. In certain embodiments, the present disclosure provides a chirally controlled oligonucleotide composition comprising a plurality of oligonucleotides, wherein with in each of the oligonucleotides at least two individual internucleotidic linkages have different P-modifications relative to one another, and each of the oligonucleotide comprises a natural phosphate linkage and a phosphorothioate internucleotidic linkage. In certain embodiments, the present disclosure provides a chirally controlled oligonucleotide composition comprising a plurality of oligonucleotides, wherein with in each of the oligonucleotides at least two individual internucleotidic linkages have different P-modifications relative to one another, and each of the oligonucleotide comprises a phosphorothioate triester internucleotidic linkage. In certain embodiments, the present disclosure provides a chirally controlled oligonucleotide composition comprising a plurality of oligonucleotides, wherein with in each of the oligonucleotides at least two individual internucleotidic linkages have different P-modifications relative to one another, and each of the oligonucleotide comprises a natural phosphate linkage and a phosphorothioate triester internucleotidic linkage. In certain embodiments, the present disclosure provides a chirally controlled oligonucleotide composition comprising a plurality of oligonucleotides, wherein with in each of the oligonucleotides at least two individual internucleotidic linkages have different P-modifications relative to one another, and each of the oligonucleotide comprises a phosphorothioate internucleotidic linkage and a phosphorothioate triester internucleotidic linkage.

In some embodiments, the present disclosure provides a chirally controlled oligonucleotide composition, e.g., a chirally controlled USH2A oligonucleotide composition, comprising a plurality of oligonucleotides which share a common base sequence that is the base sequence of an oligonucleotide disclosed herein, wherein at least one internucleotidic linkage is chirally controlled. In some embodiments, the present disclosure provides a chirally controlled oligonucleotide composition, e.g., a chirally controlled USH2A oligonucleotide composition, comprising a plurality of oligonucleotides which share a common base sequence that is the base sequence of an oligonucleotide disclosed herein, wherein at least one internucleotidic linkage is chirally controlled, and at least one internucleotidic linkage has the structure of formula I as described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612 or a salt form thereof. In some embodiments, the present disclosure provides a chirally controlled oligonucleotide composition, e.g., a chirally controlled USH2A oligonucleotide composition, comprising a plurality of oligonucleotides which share a common base sequence that is the base sequence of an oligonucleotide disclosed herein, wherein at least one internucleotidic linkage is chirally controlled, and each chirally controlled internucleotidic linkage has the structure of formula I as described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612 or a salt form thereof. In some embodiments, a chirally controlled internucleotidic linkage is a chirally controlled phosphorothioate internucleotidic linkage. In some embodiments, each chirally controlled internucleotidic linkage is a chirally controlled phosphorothioate internucleotidic linkage.

### Stereochemistry and Patterns of Backbone Chiral Centers

In contrast to natural phosphate linkages, linkage phosphorus of chiral modified internucleotidic linkages, e.g., phosphorothioate internucleotidic linkages, are chiral. Among other things, the present disclosure provides technologies (e.g., oligonucleotides, compositions, methods, etc.) comprising control of stereochemistry of chiral linkage phosphorus in chiral internucleotidic linkages. In some embodiments, as demonstrated herein, control of stereochemistry can provide improved properties and/or activities, including desired stability, reduced toxicity, improved reduction of target nucleic acids, etc. In some embodiments, the present disclosure provides useful patterns of backbone chiral centers for oligonucleotides and/or regions thereof, which pattern is a combination of stereochemistry of each chiral linkage phosphorus (Rp or Sp) of chiral linkage phosphorus, indication of each achiral linkage phosphorus (Op, if any), etc. from 5′ to 3′. In some embodiments, patterns of backbone chiral centers can control cleavage patterns of target nucleic acids when they are contacted with provided oligonucleotides or compositions thereof in a cleavage system (e.g., in vitro assay, cells, tissues, organs, organisms, subjects, etc.). In some embodiments, patterns of backbone chiral centers improve cleavage efficiency and/or selectivity of target nucleic acids when they are contacted with provided oligonucleotides or compositions thereof in a cleavage system.

In some embodiments, a pattern of backbone chiral centers of an USH2A oligonucleotide or a region thereof comprises or is (Np)n(Op)m, wherein Np is Rp or Sp, Op represents a linkage phosphorus being achiral (e.g., as for the linkage phosphorus of natural phosphate linkages), and each of n and m is independently 1-50. In some embodiments, a pattern of backbone chiral centers of an USH2A oligonucleotide or a region thereof comprises or is (Rp)n(Sp)m, wherein each of n and m is independently as defined and described in the present disclosure. In some embodiments, a pattern of backbone chiral centers of an USH2A oligonucleotide or a region thereof comprises or is Rp(Sp)m, wherein each of n and m is independently as defined and described in the present disclosure. In some embodiments, a pattern of backbone chiral centers of an oligonucleotide or a region thereof comprises or is (Sp)n(Op)m, wherein each variable is independently as defined and described in the present disclosure. In some embodiments, a pattern of backbone chiral centers of an oligonucleotide or a region thereof comprises or is (Rp)n(Op)m, wherein each variable is independently as defined and described in the present disclosure. In some embodiments, n is 1. In some embodiments, a pattern of backbone chiral centers of an oligonucleotide or a region thereof comprises or is (Sp)(Op)m, wherein m is 1, 2, 3, 4, 5, 6, 7, 8, 9, or 10. In some embodiments, a pattern of backbone chiral centers of an oligonucleotide or a region thereof comprises or is (Rp)(Op)m, wherein m is 1, 2, 3, 4, 5, 6, 7, 8, 9, or 10. In some embodiments, as described in the present disclosure, m is 2; in some embodiments, m is 3; in some embodiments, m is 4; in some embodiments, m is 5; in some embodiments, m is 6.

In some embodiments, a pattern of backbone chiral centers of an USH2A oligonucleotide or a region thereof comprises or is (Op)m(Np)n, wherein Np is Rp or Sp, Op represents a linkage phosphorus being achiral (e.g., as for the linkage phosphorus of natural phosphate linkages), and each of n and m is independently as defined and described in the present disclosure. In some embodiments, a pattern of backbone chiral centers of an oligonucleotide or a region thereof comprises or is (Op)m(Sp)n, wherein each variable is independently as defined and described in the present disclosure. In some embodiments, a pattern of backbone chiral centers of an oligonucleotide or a region thereof comprises or is (Op)m(Rp)n, wherein each variable is independently as defined and described in the present disclosure. In some embodiments, n is 1. In some embodiments, a pattern of backbone chiral centers of an oligonucleotide or a region thereof comprises or is (Op)m(Sp), wherein m is 1, 2, 3, 4, 5, 6, 7, 8, 9, or 10. In some embodiments, a pattern of backbone chiral centers of an oligonucleotide or a region thereof comprises or is (Op)m(Rp), wherein m is 1, 2, 3, 4, 5, 6, 7, 8, 9, or 10. In some embodiments, as described in the present disclosure, m is 2; in some embodiments, m is 3; in some embodiments, m is 4; in some embodiments, m is 5; in some embodiments, m is 6.

In some embodiments, at least one or each Rp is the configuration of a chiral non-negatively charged internucleotidic linkage, e.g., n001.

In some embodiments, a pattern of backbone chiral centers of an USH2A oligonucleotide or a region thereof comprises or is (Sp)m(Rp/Op)n or (Rp/Op)n(Sp)m, wherein each variable is independently as described in the present disclosure. Non-limiting examples of such an oligonucleotide (wherein Rp/Op is Op) include but are not limited to: WV-24393, WV-24392, WV-24391, WV-24390, WV-24389, WV-24388, WV-24387, WV-24386, WV-24373, WV-24372, WV-24371, WV-24370, WV-24369, WV-24368, WV-24367, WV-24366, WV-24365, WV-24364, WV-24363, WV-24362, WV-24361, WV-24360, WV-24359, WV-24358, WV-24357, WV-24356, WV-21105, WV-21104, WV-21103, WV-21099, WV-21098, and WV-21097.

In some embodiments, m is 1, 2, 3, 4, 5, 6, 7, 8, 9, 10, 11, 12, 13, 14, 15, 16, 17, 18, 19, 20, 21, 22, 23, 24, or 25. In some embodiments, in a pattern of backbone chiral centers each m is independently 2 or more. In some embodiments, each m is independently 2, 3, 4, 5, 6, 7, 8, 9, or 10. In some embodiments, each m is independently 2-3, 2-5, 2-6, or 2-10. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10. In some embodiments, where there are two or more occurrences of m, they can be the same or different, and each of them is independently as described in the present disclosure.

In some embodiments, y is 1, 2, 3, 4, 5, 6, 7, 8, 9, 10, 11, 12, 13, 14, 15, 16, 17, 18, 19, 20, 21, 22, 23, 24, or 25. In some embodiments, y is 1, 2, 3, 4, 5, 6, 7, 8, 9, or 10. In some embodiments, y is 1. In some embodiments, y is 2. In some embodiments, y is 3. In some embodiments, y is 4. In some embodiments, y is 5. In some embodiments, y is 6. In some embodiments, y is 7. In some embodiments, y is 8. In some embodiments, y is 9. In some embodiments, y is 10.

In some embodiments, t is 1, 2, 3, 4, 5, 6, 7, 8, 9, 10, 11, 12, 13, 14, 15, 16, 17, 18, 19, 20, 21, 22, 23, 24, or 25. In some embodiments, each t is independently 1, 2, 3, 4, 5, 6, 7, 8, 9, or 10. In some embodiments, t is 2 or more. In some embodiments, t is 1. In some embodiments, t is 2. In some embodiments, t is 3. In some embodiments, t is 4. In some embodiments, t is 5. In some embodiments, t is 6. In some embodiments, t is 7. In some embodiments, t is 8. In some embodiments, t is 9. In some embodiments, t is 10. In some embodiments, where there are two or more occurrences of t, they can be the same or different, and each of them is independently as described in the present disclosure.

In some embodiments, n is 1, 2, 3, 4, 5, 6, 7, 8, 9, 10, 11, 12, 13, 14, 15, 16, 17, 18, 19, 20, 21, 22, 23, 24, or 25. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10. In some embodiments, where there are two or more occurrences of n, they can be the same or different, and each of them is independently as described in the present disclosure. In many embodiments, in a pattern of backbone chiral centers, at least one occurrence of n is 1; in some cases, each n is 1.

In some embodiments, k is 1, 2, 3, 4, 5, 6, 7, 8, 9, 10, 11, 12, 13, 14, 15, 16, 17, 18, 19, 20, 21, 22, 23, 24, or 25. In some embodiments, k is 1. In some embodiments, k is 2. In some embodiments, k is 3. In some embodiments, k is 4. In some embodiments, k is 5. In some embodiments, k is 6. In some embodiments, k is 7. In some embodiments, k is 8. In some embodiments, k is 9. In some embodiments, k is 10.

In some embodiments, f is 1-20. In some embodiments, f is 1-10. In some embodiments, f is 1-5. In some embodiments, f is 1, 2, 3, 4, 5, 6, 7, 8, 9, 10, 11, 12, 13, 14, 15, 16, 17, 18, 19, 20, 21, 22, 23, 24, or 25. In some embodiments, f is 1. In some embodiments, f is 2. In some embodiments, f is 3. In some embodiments, f is 4. In some embodiments, f is 5. In some embodiments, f is 6. In some embodiments, f is 7. In some embodiments, f is 8. In some embodiments, f is 9. In some embodiments, f is 10.

In some embodiments, g is 1-20. In some embodiments, g is 1-10. In some embodiments, g is 1-5. In some embodiments, g is 2-10. In some embodiments, g is 2-5. In some embodiments, g is 1, 2, 3, 4, 5, 6, 7, 8, 9, 10, 11, 12, 13, 14, 15, 16, 17, 18, 19, 20, 21, 22, 23, 24, or 25. In some embodiments, g is 1. In some embodiments, g is 2. In some embodiments, g is 3. In some embodiments, g is 4. In some embodiments, g is 5. In some embodiments, g is 6. In some embodiments, g is 7. In some embodiments, g is 8. In some embodiments, g is 9. In some embodiments, g is 10.

In some embodiments, h is 1-20. In some embodiments, h is 1-10. In some embodiments, h is 1-5. In some embodiments, h is 2-10. In some embodiments, h is 2-5. In some embodiments, h is 1, 2, 3, 4, 5, 6, 7, 8, 9, 10, 11, 12, 13, 14, 15, 16, 17, 18, 19, 20, 21, 22, 23, 24, or 25. In some embodiments, h is 1. In some embodiments, h is 2. In some embodiments, h is 3. In some embodiments, h is 4. In some embodiments, h is 5. In some embodiments, h is 6. In some embodiments, h is 7. In some embodiments, h is 8. In some embodiments, h is 9. In some embodiments, h is 10.

In some embodiments, j is 1-20. In some embodiments, j is 1-10. In some embodiments, j is 1-5. In some embodiments, j is 1, 2, 3, 4, 5, 6, 7, 8, 9, 10, 11, 12, 13, 14, 15, 16, 17, 18, 19, 20, 21, 22, 23, 24, or 25. In some embodiments, j is 1. In some embodiments, j is 2. In some embodiments, j is 3. In some embodiments, j is 4. In some embodiments, j is 5. In some embodiments, j is 6. In some embodiments, j is 7. In some embodiments, j is 8. In some embodiments, j is 9. In some embodiments, j is 10.

In some embodiments, at least one n is 1, and at least one m is no less than 2. In some embodiments, at least one n is 1, at least one t is no less than 2, and at least one m is no less than 3. In some embodiments, each n is 1. In some embodiments, t is 1. In some embodiments, at least one t>1. In some embodiments, at least one t>2. In some embodiments, at least one t>3. In some embodiments, at least one t>4. In some embodiments, at least one m>1. In some embodiments, at least one m>2. In some embodiments, at least one m>3. In some embodiments, at least one m>4. In some embodiments, a pattern of backbone chiral centers comprises one or more achiral natural phosphate linkages. In some embodiments, the sum of m, t, and n (or m and n if not in a pattern) is no less than 5, 6, 7, 8, 9, 10, 11, 12, 13, 14, 15, 16, 17, 18, 19 or 20. In some embodiments, the sum is 5. In some embodiments, the sum is 6. In some embodiments, the sum is 7. In some embodiments, the sum is 8. In some embodiments, the sum is 9. In some embodiments, the sum is 10. In some embodiments, the sum is 11. In some embodiments, the sum is 12. In some embodiments, the sum is 13. In some embodiments, the sum is 14. In some embodiments, the sum is 15.

In some embodiments, a number of linkage phosphorus in chirally controlled internucleotidic linkages are Sp. In some embodiments, at least 10%, 20%, 25%, 30%, 35%, 40%, 45%, 50%, 55%, 60%, 65%, 70%, 75%, 80%, 85%, 90% or 95% of chirally controlled internucleotidic linkages have Sp linkage phosphorus. In some embodiments, at least 10%, 20%, 25%, 30%, 35%, 40%, 45%, 50%, 55%, 60%, 65%, 70%, 75%, 80%, 85%, 90% or 95% of chirally controlled phosphorothioate internucleotidic linkages have Sp linkage phosphorus. In some embodiments, at least 10%, 20%, 25%, 30%, 35%, 40%, 45%, 50%, 55%, 60%, 65%, 70%, 75%, 80%, 85%, 90% or 95% of all chiral internucleotidic linkages are chirally controlled internucleotidic linkages having Sp linkage phosphorus. In some embodiments, at least 10%, 20%, 25%, 30%, 35%, 40%, 45%, 50%, 55%, 60%, 65%, 70%, 75%, 80%, 85%, 90% or 95% of all chiral internucleotidic linkages are chirally controlled phosphorothioate internucleotidic linkages having Sp linkage phosphorus. In some embodiments, at least 10%, 20%, 25%, 30%, 35%, 40%, 45%, 50%, 55%, 60%, 65%, 70%, 75%, 80%, 85%, 90% or 95% of all internucleotidic linkages are chirally controlled internucleotidic linkages having Sp linkage phosphorus. In some embodiments, at least 10%, 20%, 25%, 30%, 35%, 40%, 45%, 50%, 55%, 60%, 65%, 70%, 75%, 80%, 85%, 90% or 95% of chirally controlled non-negatively charged internucleotidic linkages (e.g., neutral internucleotidic linkages, n001, etc.) have Rp linkage phosphorus. In some embodiments, the percentage is at least 20%. In some embodiments, the percentage is at least 30%. In some embodiments, the percentage is at least 40%. In some embodiments, the percentage is at least 50%. In some embodiments, the percentage is at least 60%. In some embodiments, the percentage is at least 65%. In some embodiments, the percentage is at least 70%. In some embodiments, the percentage is at least 75%. In some embodiments, the percentage is at least 80%. In some embodiments, the percentage is at least 90%. In some embodiments, the percentage is at least 95%. In some embodiments, at least 1, 2, 3, 4, 5, 6, 7, 8, 9, 10, 11, 12, 13, 14, 15, 16, 17, 18, 19, 20, 21, 22, 23, 24, or 25 internucleotidic linkages are chirally controlled internucleotidic linkages having Sp linkage phosphorus. In some embodiments, at least 5 internucleotidic linkages are chirally controlled internucleotidic linkages having Sp linkage phosphorus. In some embodiments, at least 6 internucleotidic linkages are chirally controlled internucleotidic linkages having Sp linkage phosphorus. In some embodiments, at least 7 internucleotidic linkages are chirally controlled internucleotidic linkages having Sp linkage phosphorus. In some embodiments, at least 8 internucleotidic linkages are chirally controlled internucleotidic linkages having Sp linkage phosphorus. In some embodiments, at least 9 internucleotidic linkages are chirally controlled internucleotidic linkages having Sp linkage phosphorus. In some embodiments, at least 10 internucleotidic linkages are chirally controlled internucleotidic linkages having Sp linkage phosphorus. In some embodiments, at least 11 internucleotidic linkages are chirally controlled internucleotidic linkages having Sp linkage phosphorus. In some embodiments, at least 12 internucleotidic linkages are chirally controlled internucleotidic linkages having Sp linkage phosphorus. In some embodiments, at least 13 internucleotidic linkages are chirally controlled internucleotidic linkages having Sp linkage phosphorus. In some embodiments, at least 14 internucleotidic linkages are chirally controlled internucleotidic linkages having Sp linkage phosphorus. In some embodiments, at least 15 internucleotidic linkages are chirally controlled internucleotidic linkages having Sp linkage phosphorus. In some embodiments, at least 1, 2, 3, 4, 5, 6, 7, 8, 9, 10, 11, 12, 13, 14, 15, 16, 17, 18, 19, 20, 21, 22, 23, 24, or 25 internucleotidic linkages are chirally controlled internucleotidic linkages having Rp linkage phosphorus. In some embodiments, no more than 1, 2, 3, 4, 5, 6, 7, 8, 9, 10, 11, 12, 13, 14, 15, 16, 17, 18, 19, 20, 21, 22, 23, 24, or 25 internucleotidic linkages are chirally controlled internucleotidic linkages having Rp linkage phosphorus. In some embodiments, one and no more than one internucleotidic linkage in an oligonucleotide is a chirally controlled internucleotidic linkage having Rp linkage phosphorus. In some embodiments, 2 and no more than 2 internucleotidic linkages in an oligonucleotide are chirally controlled internucleotidic linkages having Rp linkage phosphorus. In some embodiments, 3 and no more than 3 internucleotidic linkages in an oligonucleotide are chirally controlled internucleotidic linkages having Rp linkage phosphorus. In some embodiments, 4 and no more than 4 internucleotidic linkages in an oligonucleotide are chirally controlled internucleotidic linkages having Rp linkage phosphorus. In some embodiments, 5 and no more than 5 internucleotidic linkages in an oligonucleotide are chirally controlled internucleotidic linkages having Rp linkage phosphorus. In some embodiments, each Rp chirally controlled internucleotidic linkage is independently a non-negatively charged internucleotidic linkage. In some embodiments, each Rp chirally controlled internucleotidic linkage is independently a neutral internucleotidic linkage. In some embodiments, each Rp chirally controlled internucleotidic linkage is independently n001. In some embodiments, each non-negatively charged internucleotidic linkage is n001.

In some embodiments, an oligonucleotide comprises one or more Rp internucleotidic linkages. In some embodiments, an oligonucleotide comprises one and no more than one Rp internucleotidic linkages. In some embodiments, an oligonucleotide comprises two or more Rp internucleotidic linkages. In some embodiments, an oligonucleotide comprises three or more Rp internucleotidic linkages. In some embodiments, an oligonucleotide comprises four or more Rp internucleotidic linkages. In some embodiments, an oligonucleotide comprises five or more Rp internucleotidic linkages. In some embodiments, about 5%-50% of all chirally controlled internucleotidic linkages in an oligonucleotide are Rp. In some embodiments, about 5%-40% of all chirally controlled internucleotidic linkages in an oligonucleotide are Rp. In some embodiments, about 10%-40% of all chirally controlled internucleotidic linkages in an oligonucleotide are Rp. In some embodiments, about 15%-40% of all chirally controlled internucleotidic linkages in an oligonucleotide are Rp. In some embodiments, about 20%-40% of all chirally controlled internucleotidic linkages in an oligonucleotide are Rp. In some embodiments, about 25%-40% of all chirally controlled internucleotidic linkages in an oligonucleotide are Rp. In some embodiments, about 30%-40% of all chirally controlled internucleotidic linkages in an oligonucleotide are Rp. In some embodiments, about 35%-40% of all chirally controlled internucleotidic linkages in an oligonucleotide are Rp.

In some embodiments, instead of an Rp internucleotidic linkage, a natural phosphate linkage may be similarly utilized, optionally with a modification, e.g., a sugar modification (e.g., a 5′-modification such as R^{5s} as described herein). In some embodiments, a modification improves stability of a natural phosphate linkage.

In some embodiments, the present disclosure provides an oligonucleotide having a pattern of backbone chiral centers as described herein. In some embodiments, oligonucleotides in a chirally controlled oligonucleotide composition share a common pattern of backbone chiral centers as described herein.

In some embodiments, at least about 25% of the internucleotidic linkages of an USH2A oligonucleotide are chirally controlled and have Sp linkage phosphorus. In some embodiments, at least about 30% of the internucleotidic linkages of an oligonucleotide are chirally controlled and have Sp linkage phosphorus. In some embodiments, at least about 40% of the internucleotidic linkages of a provided oligonucleotide are chirally controlled and have Sp linkage phosphorus. In some embodiments, at least about 50% of the internucleotidic linkages of a provided oligonucleotide are chirally controlled and have Sp linkage phosphorus. In some embodiments, at least about 60% of the internucleotidic linkages of a provided oligonucleotide are chirally controlled and have Sp linkage phosphorus. In some embodiments, at least about 65% of the internucleotidic linkages of a provided oligonucleotide are chirally controlled and have Sp linkage phosphorus. In some embodiments, at least about 70% of the internucleotidic linkages of a provided oligonucleotide are chirally controlled and have Sp linkage phosphorus. In some embodiments, at least about 75% of the internucleotidic linkages of a provided oligonucleotide are chirally controlled and have Sp linkage phosphorus. In some embodiments, at least about 80% of the internucleotidic linkages of a provided oligonucleotide are chirally controlled and have Sp linkage phosphorus. In some embodiments, at least about 85% of the internucleotidic linkages of a provided oligonucleotide are chirally controlled and have Sp linkage phosphorus. In some embodiments, at least about 90% of the internucleotidic linkages of a provided oligonucleotide are chirally controlled and have Sp linkage phosphorus. In some embodiments, at least about 95% of the internucleotidic linkages of a provided oligonucleotide are chirally controlled and have Sp linkage phosphorus.

In some embodiments, the present disclosure provides chirally controlled oligonucleotide compositions, e.g., chirally controlled USH2A oligonucleotide compositions, wherein the composition comprises a non-random or controlled level of a plurality of oligonucleotides, wherein oligonucleotides of the plurality share a common base sequence, and share the same configuration of linkage phosphorus independently at 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, 21, 22, 23, 24, or 25 or more chiral internucleotidic linkages.

In some embodiments, provided oligonucleotides comprise 2-30 chirally controlled internucleotidic linkages. In some embodiments, provided oligonucleotide compositions comprise 5-30 chirally controlled internucleotidic linkages. In some embodiments, provided oligonucleotide compositions comprise 10-30 chirally controlled internucleotidic linkages. In some embodiments, provided oligonucleotide compositions comprise 1, 2, 3, 4, 5, 6, 7, 8, 9, 10, 11, 12, 13, 14, 15, 16, 17, 18, 19, 20, 21, 22, 23, 24, or 25 or more chirally controlled internucleotidic linkages.

In some embodiments, about 1-100% of all internucleotidic linkages are chirally controlled internucleotidic linkages. In some embodiments, a percentage is about 5%-100%. In some embodiments, a percentage is at least 5%, 10%, 15%, 20%, 25%, 30%, 35%, 40%, 45%, 50%, 55%, 60%, 65%, 70%, 75%, 80%, 85%, 90%, 95%, 965, 96%, 98%, or 99%. In some embodiments, a percentage is about 5%, 10%, 15%, 20%, 25%, 30%, 35%, 40%, 45%, 50%, 55%, 60%, 65%, 70%, 75%, 80%, 85%, 90%, 95%, 965, 96%, 98%, or 99%.

In some embodiments, a pattern of backbone chiral centers in an USH2A oligonucleotide comprises a pattern of i^{o}-i^{s}-i^{o}-i^{s}-i^{o}, i^{o}-i^{s}-i^{s}-i^{s}-i^{o}, i^{o}-i^{s}-i^{s}-i^{s}-i^{o}-i^{s}, i^{s}-i^{o}-i^{s}-i^{o}, i^{s}-i^{o}-i^{s}-i^{o}, i^{s}-i^{o}-i^{s}-i^{o}-i^{s}, i^{s}-i^{o}-i^{s}-i^{o}-i^{s}-i^{o}, i^{s}-i^{o}-i^{s}-i^{o}-i^{s}-i^{o}-i^{s}-i^{o}, i^{s}-i^{o}-i^{s}-i^{s}-i^{s}-i^{o}, i^{s}-i^{s}-i^{o}-i^{s}-i^{s}-i^{s}-i^{o}-i^{s}-i^{s}, i^{s}-i^{s}-i^{s}-i^{o}-i^{s}-i^{o}-i^{s}-i^{s}-i^{s}, i^{s}-i^{s}-i^{s}-i^{s}-i^{o}-i^{s}-i^{o}-i^{s}-i^{s}-i^{s}-i^{s}, i^{s}-i^{s}-i^{s}- i^{s}-i^{s}, i^{s}-i^{s}-i^{s}-i^{s}-i^{s}-i^{s}, i^{s}-i^{s}-i^{s}-i^{s}-i^{s}-i^{s}-i^{s}, i^{s}-i^{s}-i^{s}-i^{s}-i^{s}-i^{s}-i^{s}-i^{s}, i^{s}-i^{s}-i^{s}-i^{s}-i^{s}-i^{s}-i^{s}-i^{s}-i^{s}, or i^{r}-i^{r}-i^{r}, wherein i^{s} represents an internucleotidic linkage in the Sp configuration; i^{o} represents an achiral internucleotidic linkage; and it represents an internucleotidic linkage in the Rp configuration.

In some embodiments, an internucleotidic linkage in the Sp configuration (having a Sp linkage phosphorus) is a phosphorothioate internucleotidic linkage. In some embodiments, an achiral internucleotidic linkage is a natural phosphate linkage. In some embodiments, an internucleotidic linkage in the Rp configuration (having a Rp linkage phosphorus) is a phosphorothioate internucleotidic linkage. In some embodiments, each internucleotidic linkage in the Sp configuration is a phosphorothioate internucleotidic linkage. In some embodiments, each achiral internucleotidic linkage is a natural phosphate linkage. In some embodiments, each internucleotidic linkage in the Rp configuration is a phosphorothioate internucleotidic linkage. In some embodiments, each internucleotidic linkage in the Sp configuration is a phosphorothioate internucleotidic linkage, each achiral internucleotidic linkage is a natural phosphate linkage, and each internucleotidic linkage in the Rp configuration is a phosphorothioate internucleotidic linkage.

In some embodiments, a pattern of backbone chiral centers (e.g., a pattern of backbone chiral centers in an USH2A oligonucleotide) comprises a pattern of OpSpOpSpOp, OpSpSpSpOp, OpSpSpSpOpSp, SpOpSpOp, SpOpSpOp, SpOpSpOpSp, SpOpSpOpSpOp, SpOpSpOpSpOpSpOp, SpOpSpSpSpOp, SpSpOpSpSpSpOpSpSp, SpSpSpOpSpOpSpSpSp, SpSpSpSpOpSpOpSpSpSpSp, SpSpSpSpSp, SpSpSpSpSpSp, SpSpSpSpSpSpSp, SpSpSpSpSpSpSpSp, SpSpSpSpSpSpSpSpSp, or RpRpRp, wherein each Rp and Sp is independently the linkage phosphorus configuration of a chirally controlled internucleotidic linkage (in some embodiments, each Rp and Sp is independently the linkage phosphorus configuration of a chirally controlled phosphorothioate internucleotidic linkage), and each Op independently represents linkage phosphorus being achiral in a natural phosphate linkage.

In some embodiments, at least about 25% of the oligonucleotides in a composition share a common base sequence, a common pattern of backbone linkages, and a common pattern of backbone chiral centers. In some embodiments, at least about 30% of the oligonucleotides in a composition share a common base sequence, a common pattern of backbone linkages, and a common pattern of backbone chiral centers. In some embodiments, at least about 50% of the oligonucleotides in a composition share a common base sequence, a common pattern of backbone linkages, and a common pattern of backbone chiral centers. In some embodiments, at least about 60% of the oligonucleotides in a composition share a common base sequence, a common pattern of backbone linkages, and a common pattern of backbone chiral centers. In some embodiments, at least about 70% of the oligonucleotides in a composition share a common base sequence, a common pattern of backbone linkages, and a common pattern of backbone chiral centers. In some embodiments, at least about 80% of the oligonucleotides in a composition share a common base sequence, a common pattern of backbone linkages, and a common pattern of backbone chiral centers. In some embodiments, at least about 85% of the oligonucleotides in a composition share a common base sequence, a common pattern of backbone linkages, and a common pattern of backbone chiral centers. In some embodiments, at least about 90% of the oligonucleotides in a composition share a common base sequence, a common pattern of backbone linkages, and a common pattern of backbone chiral centers. In some embodiments, at least about 92% of the oligonucleotides in a composition share a common base sequence, a common pattern of backbone linkages, and a common pattern of backbone chiral centers. In some embodiments, at least about 94% of the oligonucleotides in a composition share a common base sequence, a common pattern of backbone linkages, and a common pattern of backbone chiral centers. In some embodiments, at least about 95% of the oligonucleotides in a composition share a common base sequence, a common pattern of backbone linkages, and a common pattern of backbone chiral centers. In some embodiments, at least about 91%, 92%, 93%, 94%, 95%, 96%, 97%, 98%, or 99% of the oligonucleotides in a composition share a common base sequence, a common pattern of backbone linkages, and a common pattern of backbone chiral centers. In some embodiments, greater than about 99% of the oligonucleotides in a composition share a common base sequence, a common pattern of backbone linkages, and a common pattern of backbone chiral centers. In some embodiments, purity of a composition may be expressed as the percentage of oligonucleotides in a composition that share a common base sequence, a common pattern of backbone linkages, and a common pattern of backbone chiral centers.

In some embodiments, provided oligonucleotides, e.g., USH2A oligonucleotides, in chirally controlled oligonucleotide compositions each comprise different types of internucleotidic linkages. In some embodiments, provided oligonucleotides comprise at least one natural phosphate linkage and at least one modified internucleotidic linkage. In some embodiments, provided oligonucleotides comprise at least one natural phosphate linkage and at least two modified internucleotidic linkages. In some embodiments, provided oligonucleotides comprise at least one natural phosphate linkage and at least three modified internucleotidic linkages. In some embodiments, provided oligonucleotides comprise at least one natural phosphate linkage and at least four modified internucleotidic linkages. In some embodiments, provided oligonucleotides comprise at least one natural phosphate linkage and at least five modified internucleotidic linkages. In some embodiments, provided oligonucleotides comprise at least one natural phosphate linkage and 1, 2, 3, 4, 5, 6, 7, 8, 9, 10, 11, 12, 13, 14, 15, 16, 17, 18, 19, 20, 21, 22, 23, 24, or 25 modified internucleotidic linkages. In some embodiments, a modified internucleotidic linkage is a phosphorothioate internucleotidic linkage. In some embodiments, each modified internucleotidic linkage is a phosphorothioate internucleotidic linkage. In some embodiments, a modified internucleotidic linkage is a phosphorothioate triester internucleotidic linkage. In some embodiments, each modified internucleotidic linkage is a phosphorothioate triester internucleotidic linkage. In some embodiments, provided oligonucleotides comprise at least one natural phosphate linkage and at least 2, 3, 4, 5, 6, 7, 8, 9, 10, 11, 12, 13, 14, 15, 16, 17, 18, 19, 20, 21, 22, 23, 24, or 25 consecutive modified internucleotidic linkages. In some embodiments, provided oligonucleotides comprise at least one natural phosphate linkage and at least 2, 3, 4, 5, 6, 7, 8, 9, 10, 11, 12, 13, 14, 15, 16, 17, 18, 19, 20, 21, 22, 23, 24, or 25 consecutive phosphorothioate internucleotidic linkages. In some embodiments, provided oligonucleotides comprise at least one natural phosphate linkage and at least 2, 3, 4, 5, 6, 7, 8, 9, 10, 11, 12, 13, 14, 15, 16, 17, 18, 19, 20, 21, 22, 23, 24, or 25 consecutive phosphorothioate triester internucleotidic linkages.

In some embodiments, oligonucleotides in a chirally controlled oligonucleotide composition each comprise at least two internucleotidic linkages that have different stereochemistry and/or different P-modifications relative to one another. In some embodiments, at least two internucleotidic linkages have different stereochemistry relative to one another, and the oligonucleotides each comprise a pattern of backbone chiral centers comprising alternating linkage phosphorus stereochemistry.

In some embodiments, a phosphorothioate triester linkage comprises a chiral auxiliary, which, for example, is used to control the stereoselectivity of a reaction, e.g., a coupling reaction in an oligonucleotide synthesis cycle. In some embodiments, a phosphorothioate triester linkage does not comprise a chiral auxiliary. In some embodiments, a phosphorothioate triester linkage is intentionally maintained until and/or during the administration of the oligonucleotide composition to a subject.

In some embodiments, oligonucleotides are linked to a solid support. In some embodiments, a solid support is a support for oligonucleotide synthesis. In some embodiments, a solid support comprises glass. In some embodiments, a solid support is CPG (controlled pore glass). In some embodiments, a solid support is polymer. In some embodiments, a solid support is polystyrene. In some embodiments, the solid support is Highly Crosslinked Polystyrene (HCP). In some embodiments, the solid support is hybrid support of Controlled Pore Glass (CPG) and Highly Cross-linked Polystyrene (HCP). In some embodiments, a solid support is a metal foam. In some embodiments, a solid support is a resin. In some embodiments, oligonucleotides are cleaved from a solid support.

In some embodiments, purity, particularly stereochemical purity, and particularly diastereomeric purity of many oligonucleotides and compositions thereof wherein all other chiral centers in the oligonucleotides but the chiral linkage phosphorus centers have been stereodefined (e.g., carbon chiral centers in the sugars, which are defined in, e.g., phosphoramidites for oligonucleotide synthesis), can be controlled by stereoselectivity (as appreciated by those skilled in this art, diastereoselectivity in many cases of oligonucleotide synthesis wherein the oligonucleotide comprise more than one chiral centers) at chiral linkage phosphorus in coupling steps when forming chiral internucleotidic linkages. In some embodiments, a coupling step has a stereoselectivity (diastereoselectivity when there are other chiral centers) of 60% at the linkage phosphorus. After such a coupling step, the new internucleotidic linkage formed may be referred to have a 60% stereochemical purity (for oligonucleotides, typically diastereomeric purity in view of the existence of other chiral centers). In some embodiments, each coupling step independently has a stereoselectivity of at least 60%. In some embodiments, each coupling step independently has a stereoselectivity of at least 70%. In some embodiments, each coupling step independently has a stereoselectivity of at least 80%. In some embodiments, each coupling step independently has a stereoselectivity of at least 85%. In some embodiments, each coupling step independently has a stereoselectivity of at least 90%. In some embodiments, each coupling step independently has a stereoselectivity of at least 91%. In some embodiments, each coupling step independently has a stereoselectivity of at least 92%. In some embodiments, each coupling step independently has a stereoselectivity of at least 93%. In some embodiments, each coupling step independently has a stereoselectivity of at least 94%. In some embodiments, each coupling step independently has a stereoselectivity of at least 95%. In some embodiments, each coupling step independently has a stereoselectivity of at least 96%. In some embodiments, each coupling step independently has a stereoselectivity of at least 97%. In some embodiments, each coupling step independently has a stereoselectivity of at least 98%. In some embodiments, each coupling step independently has a stereoselectivity of at least 99%. In some embodiments, each coupling step independently has a stereoselectivity of at least 99.5%. In some embodiments, each coupling step independently has a stereoselectivity of virtually 100%. In some embodiments, a coupling step has a stereoselectivity of virtually 100% in that each detectable product from the coupling step analyzed by an analytical method (e.g., NMR, HPLC, etc.) has the intended stereoselectivity. In some embodiments, a chirally controlled internucleotidic linkage is typically formed with a stereoselectivity of at least 90%, 91%, 92%, 93%, 94%, 95%, 96%, 97%, 98%, 99.5% or virtually 100% (in some embodiments, at least 90%; in some embodiments, at least 95%; in some embodiments, at least 96%; in some embodiments, at least 97%; in some embodiments, at least 98%; in some embodiments, at least 99%). In some embodiments, a chirally controlled internucleotidic linkage has a stereochemical purity (typically diastereomeric purity for oligonucleotides with multiple chiral centers) of at least 90%, 91%, 92%, 93%, 94%, 95%, 96%, 97%, 98%, 99.5% or virtually 100% (in some embodiments, at least 90%; in some embodiments, at least 95%; in some embodiments, at least 96%; in some embodiments, at least 97%; in some embodiments, at least 98%; in some embodiments, at least 99%) at its chiral linkage phosphorus. In some embodiments, each chirally controlled internucleotidic linkage independently has a stereochemical purity (typically diastereomeric purity for oligonucleotides with multiple chiral centers) of at least 90%, 91%, 92%, 93%, 94%, 95%, 96%, 97%, 98%, 99.5% or virtually 100% (in some embodiments, at least 90%; in some embodiments, at least 95%; in some embodiments, at least 96%; in some embodiments, at least 97%; in some embodiments, at least 98%; in some embodiments, at least 99%) at its chiral linkage phosphorus. In some embodiments, a non-chirally controlled internucleotidic linkage is typically formed with a stereoselectivity of less than 60%, 70%, 80%, 85%, or 90% (in some embodiments, less than 60%; in some embodiments, less than 70%; in some embodiments, less than 80%; in some embodiments, less than 85%; in some embodiments, less than 90%). In some embodiments, each non-chirally controlled internucleotidic linkage is independently formed with a stereoselectivity of less than 60%, 70%, 80%, 85%, or 90% (in some embodiments, less than 60%; in some embodiments, less than 70%; in some embodiments, less than 80%; in some embodiments, less than 85%; in some embodiments, less than 90%). In some embodiments, a non-chirally controlled internucleotidic linkage has a stereochemical purity (typically diastereomeric purity for oligonucleotides with multiple chiral centers) of less than 60%, 70%, 80%, 85%, or 90% (in some embodiments, less than 60%; in some embodiments, less than 70%; in some embodiments, less than 80%; in some embodiments, less than 85%; in some embodiments, less than 90%) at its chiral linkage phosphorus. In some embodiments, each non-chirally controlled internucleotidic linkage independently has a stereochemical purity (typically diastereomeric purity for oligonucleotides with multiple chiral centers) of less than 60%, 70%, 80%, 85%, or 90% (in some embodiments, less than 60%; in some embodiments, less than 70%; in some embodiments, less than 80%; in some embodiments, less than 85%; in some embodiments, less than 90%) at its chiral linkage phosphorus.

In some embodiments, at least 1, 2, 3, 4, 5, 6, 7, 8, 9, or 10 couplings of a monomer (as appreciated by those skilled in the art in many embodiments a phosphoramidite for oligonucleotide synthesis) independently have a stereoselectivity less than about 60%, 70%, 80%, 85%, or 90% [for oligonucleotide synthesis, typically diastereoselectivity with respect to formed linkage phosphorus chiral center(s)]. In some embodiments, at least one coupling has a stereoselectivity less than about 60%, 70%, 80%, 85%, or 90%. In some embodiments, at least two couplings independently have a stereoselectivity less than about 60%, 70%, 80%, 85%, or 90%. In some embodiments, at least three couplings independently have a stereoselectivity less than about 60%, 70%, 80%, 85%, or 90%. In some embodiments, at least four couplings independently have a stereoselectivity less than about 60%, 70%, 80%, 85%, or 90%. In some embodiments, at least five couplings independently have a stereoselectivity less than about 60%, 70%, 80%, 85%, or 90%. In some embodiments, each coupling independently has a stereoselectivity less than about 60%, 70%, 80%, 85%, or 90%. In some embodiments, each non-chirally controlled internucleotidic linkage is independently formed with a stereoselectivity less than about 60%, 70%, 80%, 85%, or 90%. In some embodiments, a stereoselectivity is less than about 60%. In some embodiments, a stereoselectivity is less than about 70%. In some embodiments, a stereoselectivity is less than about 80%. In some embodiments, a stereoselectivity is less than about 90%. In some embodiments, at least 1, 2, 3, 4, 5, 6, 7, 8, 9, 10, 11, 12, 13, 14, 15, 16, 17, 18, 19, 20, 21, 22, 23, 24, or 25 couplings independently have a stereoselectivity less than about 90%. In some embodiments, at least one coupling has a stereoselectivity less than about 90%. In some embodiments, at least two couplings have a stereoselectivity less than about 90%. In some embodiments, at least three couplings have a stereoselectivity less than about 90%. In some embodiments, at least four couplings have a stereoselectivity less than about 90%. In some embodiments, at least five couplings have a stereoselectivity less than about 90%. In some embodiments, each coupling independently has a stereoselectivity less than about 90%. In some embodiments, at least 1, 2, 3, 4, 5, 6, 7, 8, 9, 10, 11, 12, 13, 14, 15, 16, 17, 18, 19, 20, 21, 22, 23, 24, or 25 couplings independently have a stereoselectivity less than about 85%. In some embodiments, each coupling independently has a stereoselectivity less than about 85%. In some embodiments, at least 1, 2, 3, 4, 5, 6, 7, 8, 9, 10, 11, 12, 13, 14, 15, 16, 17, 18, 19, 20, 21, 22, 23, 24, or 25 couplings independently have a stereoselectivity less than about 80%. In some embodiments, each coupling independently has a stereoselectivity less than about 80%. In some embodiments, at least 1, 2, 3, 4, 5, 6, 7, 8, 9, 10, 11, 12, 13, 14, 15, 16, 17, 18, 19, 20, 21, 22, 23, 24, or 25 couplings independently have a stereoselectivity less than about 70%. In some embodiments, each coupling independently has a stereoselectivity less than about 70%.

In some embodiments, in stereorandom (or racemic) preparations (or stereorandom/non-chirally controlled oligonucleotide compositions), at least 1, 2, 3, 4, 5, 6, 7, 8, 9, 10, 11, 12, 13, 14, 15, 16, 17, 18, 19, 20, 21, 22, 23, 24, or 25 chiral internucleotidic linkages of the oligonucleotides independently have a stereochemical purity (typically diastereomeric purity for oligonucleotides comprising multiple chiral centers) less than about 60%, 70%, 80%, 85%, or 90% with respect to chiral linkage phosphorus of the internucleotidic linkage(s). In some embodiments, at least one internucleotidic linkage has a diastereomeric purity less than about 60%, 70%, 80%, 85%, or 90%. In some embodiments, at least two internucleotidic linkages independently have a diastereomeric purity less than about 60%, 70%, 80%, 85%, or 90%. In some embodiments, at least three internucleotidic linkages independently have a diastereomeric purity less than about 60%, 70%, 80%, 85%, or 90%. In some embodiments, at least four internucleotidic linkages independently have a diastereomeric purity less than about 60%, 70%, 80%, 85%, or 90%. In some embodiments, at least five internucleotidic linkages independently have a diastereomeric purity less than about 60%, 70%, 80%, 85%, or 90%. In some embodiments, each internucleotidic linkages independently has a diastereomeric purity less than about 60%, 70%, 80%, 85%, or 90%. In some embodiments, a diastereomeric purity is less than about 60%. In some embodiments, a diastereomeric purity is less than about 70%. In some embodiments, a diastereomeric purity is less than about 80%. In some embodiments, a diastereomeric purity is less than about 85%. In some embodiments, a diastereomeric purity is less than about 90%. In some embodiments, at least 1, 2, 3, 4, 5, 6, 7, 8, 9, 10, 11, 12, 13, 14, 15, 16, 17, 18, 19, 20, 21, 22, 23, 24, or 25 internucleotidic linkages independently have a diastereomeric purity less than about 90%. In some embodiments, at least one internucleotidic linkage has a diastereomeric purity less than about 90%. In some embodiments, at least two internucleotidic linkages independently have a diastereomeric purity less than about 90%. In some embodiments, at least three internucleotidic linkages independently have a diastereomeric purity less than about 90%. In some embodiments, at least four internucleotidic linkages independently have a diastereomeric purity less than about 90%. In some embodiments, at least five internucleotidic linkages independently have a diastereomeric purity less than about 90%. In some embodiments, each chiral internucleotidic linkage internucleotidic linkage independently has a diastereomeric purity less than about 90%. In some embodiments, at least 1, 2, 3, 4, 5, 6, 7, 8, 9, 10, 11, 12, 13, 14, 15, 16, 17, 18, 19, 20, 21, 22, 23, 24, or 25 internucleotidic linkages independently have a diastereomeric purity less than about 85%. In some embodiments, each chiral internucleotidic linkage independently has a diastereomeric purity less than about 85%. In some embodiments, at least 1, 2, 3, 4, 5, 6, 7, 8, 9, 10, 11, 12, 13, 14, 15, 16, 17, 18, 19, 20, 21, 22, 23, 24, or 25 internucleotidic linkages independently have a diastereomeric purity less than about 80%. In some embodiments, each chiral internucleotidic linkage independently has a diastereomeric purity less than about 80%.

In some embodiments, at least 5%-100% of all chiral elements of provided oligonucleotides each independently have a diastereomeric purity as described herein. In some embodiments, at least 5%, 10%, 15%, 20%, 25%, 30%, 40%, 45%, 50%, 55%, 60%, 65%, 70%, 75%, 80%, 85%, 90%, 95%, or 100% of all chiral elements each independently have a diastereomeric purity as described herein. In some embodiments, at least 5%-100% of all chiral phosphorus centers each independently have a diastereomeric purity as described herein. In some embodiments, at least 5%, 10%, 15%, 20%, 25%, 30%, 40%, 45%, 50%, 55%, 60%, 65%, 70%, 75%, 80%, 85%, 90%, 95%, or 100% of all chiral linkage phosphorus each independently have a diastereomeric purity as described herein. In some embodiments, provided oligonucleotides, e.g., oligonucleotides of a plurality in provided chirally controlled oligonucleotide compositions have a diastereomeric purity as described herein.

In some embodiments, a stereochemical purity, e.g., diastereomeric purity, is about 60%-100%. In some embodiments, a diastereomeric purity, is about 60%-100%. In some embodiments, the percentage is at least 60%, 65%, 70%, 75% 80%, 85%, 90%, 91%, 92%, 93%, 93%, 95%, 96%, 97%, 98%, or 99%. In some embodiments, the percentage is at least 80%, 85%, 90%, 91%, 92%, 93%, 93%, 95%, 96%, 97%, 98%, or 99%. In some embodiments, the percentage is at least 90%, 91%, 92%, 93%, 93%, 95%, 96%, 97%, 98%, or 99%. In some embodiments, a diastereomeric purity is at least 60%. In some embodiments, a diastereomeric purity is at least 70%. In some embodiments, a diastereomeric purity is at least 80%. In some embodiments, a diastereomeric purity is at least 85%. In some embodiments, a diastereomeric purity is at least 90%. In some embodiments, a diastereomeric purity is at least 91%. In some embodiments, a diastereomeric purity is at least 92%. In some embodiments, a diastereomeric purity is at least 93%. In some embodiments, a diastereomeric purity is at least 94%. In some embodiments, a diastereomeric purity is at least 95%. In some embodiments, a diastereomeric purity is at least 96%. In some embodiments, a diastereomeric purity is at least 97%. In some embodiments, a diastereomeric purity is at least 98%. In some embodiments, a diastereomeric purity is at least 99%. In some embodiments, a diastereomeric purity is at least 99.5%.

In some embodiments, compounds of the present disclosure (e.g., oligonucleotides, chiral auxiliaries, etc.) comprise multiple chiral elements (e.g., multiple carbon and/or phosphorus (e.g., linkage phosphorus of chiral internucleotidic linkages) chiral centers). In some embodiments, at least 1, 2, 3, 4, 5, 6, 7, 8, 9 or more chiral elements of a provided compound (e.g., an oligonucleotide) each independently have a diastereomeric purity as described herein. In some embodiments, at least 1, 2, 3, 4, 5, 6, 7, 8, 9 or more chiral carbon centers of a provided compound each independently have a diastereomeric purity as described herein. In some embodiments, at least 1, 2, 3, 4, 5, 6, 7, 8, 9 or more chiral phosphorus centers of a provided compound each independently have a diastereomeric purity as described herein. In some embodiments, each chiral element independently has a diastereomeric purity as described herein. In some embodiments, each chiral center independently has a diastereomeric purity as described herein. In some embodiments, each chiral carbon center independently has a diastereomeric purity as described herein. In some embodiments, each chiral phosphorus center independently has a diastereomeric purity as described herein. In some embodiments, each chiral phosphorus center independently has a diastereomeric purity of at least 90%, 91%, 92%, 93%, 93%, 95%, 96%, 97%, 98%, or 99% or more.

As understood by a person having ordinary skill in the art, in some embodiments, diastereoselectivity of a coupling or diastereomeric purity of a chiral linkage phosphorus center can be assessed through the diastereoselectivity of a dimer formation or diastereomeric purity of a dimer prepared under the same or comparable conditions, wherein the dimer has the same 5′- and 3′-nucleosides and internucleotidic linkage.

Various technologies can be utilized for identifying or confirming stereochemistry of chiral elements (e.g., configuration of chiral linkage phosphorus) and/or patterns of backbone chiral centers, and/or for assessing stereoselectivity (e.g., diastereoselectivity of couple steps in oligonucleotide synthesis) and/or stereochemical purity (e.g., diastereomeric purity of internucleotidic linkages, compounds (e.g., oligonucleotides), etc.). Example technologies include NMR [e.g., 1D (one-dimensional) and/or 2D (two-dimensional) ¹H-³¹P HETCOR (heteronuclear correlation spectroscopy)], HPLC, RP-HPLC, mass spectrometry, LC-MS, and cleavage of internucleotidic linkages by stereospecific nucleases, etc., which may be utilized individually or in combination. Example useful nucleases include benzonase, micrococcal nuclease, and svPDE (snake venom phosphodiesterase), which are specific for certain internucleotidic linkages with Rp linkage phosphorus (e.g., a Rp phosphorothioate linkage); and nuclease P1, mung bean nuclease, and nuclease S1, which are specific for internucleotidic linkages with Sp linkage phosphorus (e.g., a Sp phosphorothioate linkage). Without wishing to be bound by any particular theory, the present disclosure notes that, in at least some cases, cleavage of oligonucleotides by a particular nuclease may be impacted by structural elements, e.g., chemical modifications (e.g., 2′-modifications of a sugars), base sequences, or stereochemical contexts. For example, it is observed that in some cases, benzonase and micrococcal nuclease, which are specific for internucleotidic linkages with Rp linkage phosphorus, were unable to cleave an isolated Rp phosphorothioate internucleotidic linkage flanked by Sp phosphorothioate internucleotidic linkages.

In some embodiments, oligonucleotides sharing a common base sequence, a common pattern of backbone linkages, and a common pattern of backbone chiral centers share a common pattern of backbone phosphorus modifications and a common pattern of base modifications. In some embodiments, oligonucleotide compositions sharing a common base sequence, a common pattern of backbone linkages, and a common pattern of backbone chiral centers share a common pattern of backbone phosphorus modifications and a common pattern of nucleoside modifications. In some embodiments, oligonucleotides share a common base sequence, a common pattern of backbone linkages, and a common pattern of backbone chiral centers have identical structures.

In some embodiments, the present disclosure provides an oligonucleotide composition comprising a plurality of oligonucleotides capable of mediating skipping of a deleterious exon in an USH2A transcript, wherein oligonucleotides of the plurality are of a particular oligonucleotide type, which composition is chirally controlled in that it is enriched, relative to a substantially racemic preparation of oligonucleotides having the same base sequence, for oligonucleotides of the particular oligonucleotide type.

In some embodiments, oligonucleotides having a common base sequence, a common pattern of backbone linkages, and a common pattern of backbone chiral centers have a common pattern of backbone phosphorus modifications and a common pattern of base modifications. In some embodiments, oligonucleotides having a common base sequence, a common pattern of backbone linkages, and a common pattern of backbone chiral centers have a common pattern of backbone phosphorus modifications and a common pattern of nucleoside modifications. In some embodiments, oligonucleotides having a common base sequence, a common pattern of backbone linkages, and a common pattern of backbone chiral centers have identical structures.

In some embodiments, the present disclosure provides USH2A oligonucleotide compositions comprising a plurality of oligonucleotides. In some embodiments, the present disclosure provides chirally controlled oligonucleotide compositions of USH2A oligonucleotides. In some embodiments, the present disclosure provides an USH2A oligonucleotide whose base sequence is or is complementary to an USH2A sequence disclosed herein or a portion thereof (e.g., various bases sequences in Table A1, wherein each U may be independently replaced with T and vice versa). In some embodiments, the present disclosure provides an USH2A oligonucleotide whose base sequence comprises a base sequence that is or is complementary to an USH2A sequence disclosed herein or a portion thereof (e.g., various bases sequences in Table A1). In some embodiments, the present disclosure provides an USH2A oligonucleotide whose base sequence comprises 15 contiguous bases of a base sequence that is or is complementary to an USH2A sequence disclosed herein or a portion thereof (e.g., various bases sequences in Table A1, wherein each U may be independently replaced with T and vice versa). In some embodiments, the present disclosure provides an USH2A oligonucleotide which has a base sequence comprising 15 contiguous bases with 0-3 mismatches of a base sequence that is or is complementary to an USH2A sequence disclosed herein or a portion thereof (e.g., various bases sequences in Table A1, wherein each U may be independently replaced with T and vice versa). In some embodiments, the present disclosure provides an USH2A oligonucleotide composition wherein the USH2A oligonucleotides comprise at least one chiral internucleotidic linkage which is not chirally controlled. In some embodiments, the present disclosure provides an USH2A oligonucleotide comprising a non-chirally controlled chiral internucleotidic linkage, wherein the base sequence of the USH2A oligonucleotide comprises a base sequence that is or is complementary to an USH2A sequence disclosed herein or a portion thereof (e.g., various bases sequences in Table A1, wherein each U may be independently replaced with T and vice versa). In some embodiments, the present disclosure provides an USH2A oligonucleotide composition comprising a non-chirally controlled chiral internucleotidic linkage, wherein the base sequence of the USH2A oligonucleotide is a base sequence that is or is complementary to an USH2A sequence disclosed herein or a portion thereof (e.g., various bases sequences in Table A1, wherein each U may be independently replaced with T and vice versa). In some embodiments, the present disclosure provides an USH2A oligonucleotide comprising a non-chirally controlled chiral internucleotidic linkage, wherein the base sequence of the USH2A oligonucleotide comprises 15 contiguous bases of a base sequence that is or is complementary to an USH2A sequence disclosed herein or a portion thereof (e.g., various bases sequences in Table A1, wherein each U may be independently replaced with T and vice versa). In some embodiments, the present disclosure provides an USH2A oligonucleotide comprising a non-chirally controlled chiral internucleotidic linkage, wherein the base sequence of the USH2A oligonucleotides comprises 15 contiguous bases with 0-3 mismatches of a base sequence that is or is complementary to an USH2A sequence disclosed herein or a portion thereof (e.g., various bases sequences in Table A1, wherein each U may be independently replaced with T and vice versa). In some embodiments, the present disclosure provides an USH2A oligonucleotide comprising a chirally controlled chiral internucleotidic linkage, wherein the base sequence of the USH2A oligonucleotide comprises a base sequence that is or is complementary to an USH2A sequence disclosed herein or a portion thereof (e.g., various bases sequences in Table A1, wherein each U may be independently replaced with T and vice versa). In some embodiments, the present disclosure provides an USH2A oligonucleotide composition comprising a chirally controlled chiral internucleotidic linkage, wherein the base sequence of the USH2A oligonucleotide is a base sequence that is or is complementary to an USH2A sequence disclosed herein or a portion thereof (e.g., various bases sequences in Table A1, wherein each U may be independently replaced with T and vice versa). In some embodiments, the present disclosure provides an USH2A oligonucleotide comprising a chirally controlled chiral internucleotidic linkage, wherein the base sequence of the USH2A oligonucleotide comprises 15 contiguous bases of a base sequence that is or is complementary to an USH2A sequence disclosed herein or a portion thereof (e.g., various bases sequences in Table A1, wherein each U may be independently replaced with T and vice versa). In some embodiments, the present disclosure provides an USH2A oligonucleotide comprising a chirally controlled chiral internucleotidic linkage, wherein the base sequence of the USH2A oligonucleotides comprises 15 contiguous bases with 0-3 mismatches of a base sequence that is or is complementary to an USH2A sequence disclosed herein or a portion thereof (e.g., various bases sequences in Table A1, wherein each U may be independently replaced with T and vice versa).

In some embodiments, oligonucleotides of the same oligonucleotide type have a common pattern of backbone phosphorus modifications and a common pattern of nucleoside modifications. In some embodiments, oligonucleotides of the same oligonucleotide type have a common pattern of sugar modifications. In some embodiments, oligonucleotides of the same oligonucleotide type have a common pattern of base modifications. In some embodiments, oligonucleotides of the same oligonucleotide type have a common pattern of nucleoside modifications. In some embodiments, oligonucleotides of the same oligonucleotide type have the same constitution. In many embodiments, oligonucleotides of the same oligonucleotide type are identical.

In some embodiments, a plurality of oligonucleotides or oligonucleotides of a particular oligonucleotide type in a provided oligonucleotide composition are USH2A oligonucleotides. In some embodiments, the present disclosure provides a chirally controlled USH2A oligonucleotide composition comprising a plurality of USH2A oligonucleotides, wherein the oligonucleotides share:
1) a common base sequence;
2) a common pattern of backbone linkages; and
3) the same linkage phosphorus stereochemistry at one or more (e.g., 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, or more) chiral internucleotidic linkages (chirally controlled internucleotidic linkages),
wherein the composition is enriched, relative to a substantially racemic preparation of oligonucleotides sharing the common base sequence and pattern of backbone linkages, for oligonucleotides of the plurality.

In some embodiments, as used herein, “one or more” or “at least one” is 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, or more.

In some embodiments, the present disclosure provides a chirally controlled USH2A oligonucleotide composition comprising a plurality of oligonucleotides, wherein the oligonucleotides share:
1) a common base sequence;
2) a common pattern of backbone linkages; and
3) a common pattern of backbone chiral centers, which composition is a substantially pure preparation of a single oligonucleotide in that at least about 10%, 15%, 20%, 25%, 30%, 35%, 40%, 45%, 50%, 55%, 60%, 65%, 70%, 75%, 80%, 85%, 90%, 91%, 92%, 93%, 93%, 95%, 96%, 97%, 98%, or 99% of the oligonucleotides in the composition have the common base sequence, the common pattern of backbone linkages, and the common pattern of backbone chiral centers.

In some embodiments, an oligonucleotide type is further defined by: 4) additional chemical moiety, if any.

In some embodiments, the percentage is at least about 10%. In some embodiments, the percentage is at least about 20%. In some embodiments, the percentage is at least about 30%. In some embodiments, the percentage is at least about 40%. In some embodiments, the percentage is at least about 50%. In some embodiments, the percentage is at least about 60%. In some embodiments, the percentage is at least about 70%. In some embodiments, the percentage is at least about 75%. In some embodiments, the percentage is at least about 80%. In some embodiments, the percentage is at least about 85%. In some embodiments, the percentage is at least about 90%. In some embodiments, the percentage is at least about 91%. In some embodiments, the percentage is at least about 92%. In some embodiments, the percentage is at least about 93%. In some embodiments, the percentage is at least about 94%. In some embodiments, the percentage is at least about 95%. In some embodiments, the percentage is at least about 96%. In some embodiments, the percentage is at least about 97%. In some embodiments, the percentage is at least about 98%. In some embodiments, the percentage is at least about 99%. In some embodiments, the percentage is or is greater than (DS)^{nc}, wherein DS and nc are each independently as described in the present disclosure.

In some embodiments, a plurality of oligonucleotides, e.g., USH2A oligonucleotides, share the same constitution. In some embodiments, a plurality of oligonucleotides, e.g., USH2A oligonucleotides, are identical (the same stereoisomer). In some embodiments, a chirally controlled oligonucleotide composition, e.g., a chirally controlled USH2A oligonucleotide composition, is a stereopure oligonucleotide composition wherein oligonucleotides of the plurality are identical (the same stereoisomer), and the composition does not contain any other stereoisomers. Those skilled in the art will appreciate that one or more other stereoisomers may exist as impurities as processes, selectivities, purifications, etc. may not achieve completeness.

In some embodiments, a provided composition is characterized in that when it is contacted with a target nucleic acid [e.g., an USH2A gene transcript (e.g., pre-mRNA, mature mRNA, other types of RNA, etc. that hybridizes with oligonucleotides of the composition)], levels of the target nucleic acid and/or a product encoded thereby is reduced compared to that observed under a reference condition. In some embodiments, a reference condition is selected from the group consisting of absence of the composition, presence of a reference composition, and combinations thereof. In some embodiments, a reference condition is absence of the composition. In some embodiments, a reference condition is presence of a reference composition. In some embodiments, a reference composition is a composition whose oligonucleotides do not hybridize with the target nucleic acid. In some embodiments, a reference composition is a composition whose oligonucleotides do not comprise a sequence that is sufficiently complementary to the target nucleic acid. In some embodiments, a provided composition is a chirally controlled oligonucleotide composition and a reference composition is a non-chirally controlled oligonucleotide composition which is otherwise identical but is not chirally controlled (e.g., a racemic preparation of oligonucleotides of the same constitution as oligonucleotides of a plurality in the chirally controlled oligonucleotide composition).

In some embodiments, the present disclosure provides a chirally controlled USH2A oligonucleotide composition comprising a plurality of USH2A oligonucleotides capable of mediating skipping of a deleterious exon in an USH2A transcript, wherein the oligonucleotides share:
1) a common base sequence,
2) a common pattern of backbone linkages, and
3) the same linkage phosphorus stereochemistry at one or more (e.g., 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, or more) chiral internucleotidic linkages (chirally controlled internucleotidic linkages),
wherein the composition is enriched, relative to a substantially racemic preparation of oligonucleotides sharing the common base sequence and pattern of backbone linkages, for oligonucleotides of the plurality,
the oligonucleotide composition being characterized in that, when it is contacted with an USH2A gene transcript in an USH2A splicing system, skipping of a deleterious exon in an USH2A gene transcript is improved relative to that observed under reference conditions selected from the group consisting of absence of the composition, presence of a reference composition, and combinations thereof.

As noted above and understood in the art, in some embodiments, the base sequence of an oligonucleotide may refer to the identity and/or modification status of nucleoside residues (e.g., of sugar and/or base components, relative to standard naturally occurring nucleotides such as adenine, cytosine, guanosine, thymine, and uracil) in the oligonucleotide and/or to the hybridization character (i.e., the ability to hybridize with particular complementary residues) of such residues.

As demonstrated herein, oligonucleotide structural elements (e.g., patterns of sugar modifications, backbone linkages, backbone chiral centers, backbone phosphorus modifications, etc.) and combinations thereof can provide surprisingly improved properties and/or bioactivities.

In some embodiments, oligonucleotide compositions are capable of reducing the expression, level and/or activity of a gene transcript or a gene product thereof (e.g., an USH2A gene transcript comprising a deleterious exon or a protein translated therefrom), for example, by altering mRNA splicing.

In some embodiments, an oligonucleotide composition, e.g., an USH2A oligonucleotide composition, is a substantially pure preparation of a single oligonucleotide stereoisomer, e.g., an USH2A oligonucleotide stereoisomer, in that oligonucleotides in the composition that are not of the oligonucleotide stereoisomer are impurities from the preparation process of said oligonucleotide stereoisomer, in some case, after certain purification procedures.

In some embodiments, the present disclosure provides oligonucleotides and oligonucleotide compositions that are chirally controlled, and in some embodiments, stereopure. For instance, in some embodiments, a provided composition contains non-random or controlled levels of one or more individual oligonucleotide types. In some embodiments, oligonucleotides of the same oligonucleotide type are identical.

### Sugars

Various sugars, including modified sugars, can be utilized in accordance with the present disclosure. In some embodiments, the present disclosure provides sugar modifications and patterns thereof optionally in combination with other structural elements (e.g., internucleotidic linkage modifications and patterns thereof, pattern of backbone chiral centers thereof, etc.) that when incorporated into oligonucleotides can provide improved properties and/or activities.

The most common naturally occurring nucleosides comprise ribose sugars (e.g., in RNA) or deoxyribose sugars (e.g., in DNA) linked to the nucleobases adenosine (A), cytosine (C), guanine (G), thymine (T) or uracil (U). In some embodiments, a sugar, e.g., various sugars in many oligonucleotides in Table A1 (unless otherwise notes), is a natural DNA sugar (in DNA nucleic acids or oligonucleotides, having the structure of

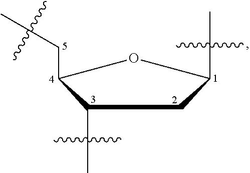

wherein a nucleobase is attached to the 1′ position, and the 3′ and 5′ positions are connected to internucleotidic linkages (as appreciated by those skilled in the art, if at the 5′-end of an oligonucleotide, the 5′ position may be connected to a 5′-end group (e.g., —OH), and if at the 3′-end of an oligonucleotide, the 3′ position may be connected to a 3′-end group (e.g., —OH). In some embodiments, a sugar is a natural RNA sugar (in RNA nucleic acids or oligonucleotides, having the structure of

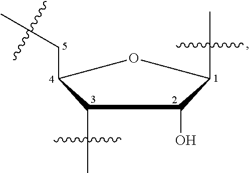

wherein a nucleobase is attached to the 1′ position, and the 3′ and 5′ positions are connected to internucleotidic linkages (as appreciated by those skilled in the art, if at the 5′-end of an oligonucleotide, the 5′ position may be connected to a 5′-end group (e.g., —OH), and if at the 3′-end of an oligonucleotide, the 3′ position may be connected to a 3′-end group (e.g., —OH). In some embodiments, a sugar is a modified sugar in that it is not a natural DNA sugar or a natural RNA sugar. Among other things, modified sugars may provide improved stability. In some embodiments, modified sugars can be utilized to alter and/or optimize one or more hybridization characteristics. In some embodiments, modified sugars can be utilized to alter and/or optimize target recognition. In some embodiments, modified sugars can be utilized to optimize Tm. In some embodiments, modified sugars can be utilized to improve oligonucleotide activities.

Sugars can be bonded to internucleotidic linkages at various positions. As non-limiting examples, internucleotidic linkages can be bonded to the 2′, 3′, 4′ or 5′ positions of sugars. In some embodiments, as most commonly in natural nucleic acids, an internucleotidic linkage connects with one sugar at the 5′ position and another sugar at the 3′ position unless otherwise indicated.

In some embodiments, a sugar is an optionally substituted natural DNA or RNA sugar. In some embodiments, a sugar is optionally substituted

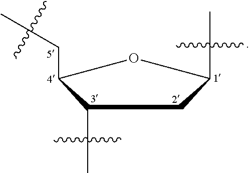

In some embodiments, the 2′ position is optionally substituted. In some embodiments, a sugar is

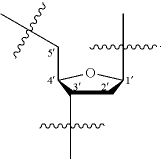

In some embodiments, a sugar has the structure of

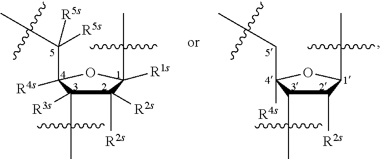

wherein each of R^{1s}, R^{2s}, R^{3s}, R^{4s}, and R^{5s} is independently —H, a suitable substituent or suitable sugar modification (e.g., those described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO2019/032612, WO 2019/055951, and/or WO 2019/075357, the substituents, sugar modifications, descriptions of R^{1s}, R^{2s}, R^{3s}, R^{4s}, and R^{5s}, and modified sugars of each of which are independently incorporated herein by reference). In some embodiments, each of R^{1s}, R^{2s}, R^{3s}, R^{4s}, and R^{5s} is independently R^{s}, wherein each R^{s} is independently —F, —Cl, —Br, —I, —CN, —N₃, —NO, —NO₂, -L^{s}-R′-L^{s}-OR, -L^{s}-SR, -L^{s}-N(R′)₂, —O-L^{s}-OR′, —O-L^{s}-SR′, or —O-L^{s}-N(R′)₂, wherein each R′ is independently as described herein, and each L^{s} is independently a covalent bond or optionally substituted bivalent C₁₋₆ aliphatic or heteroaliphatic having 1-4 heteroatoms; or two R^{s} are taken together to form a bridge -L^{s}-. In some embodiments, R′ is optionally substituted C₁₋₁₀ aliphatic. In some embodiments, a sugar has the structure of

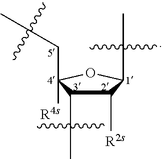

In some embodiments, R^{4s} is —H. In some embodiments, a sugar has the structure of

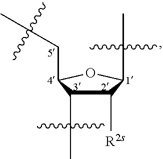

wherein R^{2s} is —H, halogen, or —OR, wherein R is optionally substituted C₁₋₆ aliphatic. In some embodiments, R^{2s} is —H. In some embodiments, R^{2s} is —F. In some embodiments, R^{2s} is —OMe. In some embodiments, R^{2s} is —OCH₂CH₂OMe.

In some embodiments, a sugar has the structure of

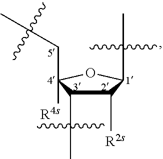

wherein R^{2s} and R^{4a} are taken together to form -L^{s}-, wherein L^{s} is a covalent bond or optionally substituted bivalent C₁₋₆ aliphatic or heteroaliphatic having 1-4 heteroatoms. In some embodiments, each heteroatom is independently selected from nitrogen, oxygen or sulfur). In some embodiments, L^{s} is optionally substituted C2-O—CH₂—C4. In some embodiments, L^{s} is C2-O—CH₂—C4. In some embodiments, L^{s} is C2-O—(R)—CH(CH₂CH₃)—C4. In some embodiments, L^{s} is C2-O—(S)—CH(CH₂CH₃)—C4.

In some embodiments, a sugar is a bicyclic sugar, e.g., sugars wherein R^{2s} and R^{4s} are taken together to form a link as described in the present disclosure. In some embodiments, a sugar is selected from LNA sugars, BNA sugars, cEt sugars, etc. In some embodiments, a bridge is between the 2′ and 4′-carbon atoms (corresponding to R^{2s} and R^{4s} taken together with their intervening atoms to form an optionally substituted ring as described herein). In some embodiments, examples of bicyclic sugars include alpha-L-methyleneoxy (4′-CH₂—O-2′) LNA, beta-D-methyleneoxy (4′-CH₂—O-2′) LNA, ethyleneoxy (4′-(CH₂)₂—O-2′) LNA, aminooxy (4′-CH₂—O—N(R)-2′) LNA, and oxyamino (4′-CH₂—N(R)—O-2′) LNA. In some embodiments, a bicyclic sugar, e.g., a LNA or BNA sugar, is sugar having at least one bridge between two sugar carbons. In some embodiments, a bicyclic sugar in a nucleoside may have the stereochemical configurations of alpha-L-ribofuranose or beta-D-ribofuranose. In some embodiments, a sugar is a sugar described in WO 1999014226. In some embodiments, a 4′-2′ bicyclic sugar or 4′ to 2′ bicyclic sugar is a bicyclic sugar comprising a furanose ring which comprises a bridge connecting the 2′ carbon atom and the 4′ carbon atom of the sugar ring. In some embodiments, a bicyclic sugar, e.g., a LNA or BNA sugar, comprises at least one bridge between two pentofuranosyl sugar carbons. In some embodiments, a LNA or BNA sugar, comprises at least one bridge between the 4′ and the 2′ pentofuranosyl sugar carbons.

In some embodiments, a bicyclic sugar is a sugar of alpha-L-methyleneoxy (4′-CH₂—O-2′) BNA, beta-D-methyleneoxy (4′-CH₂—O-2′) BNA, ethyleneoxy (4′-(CH₂)₂—O-2′) BNA, aminooxy (4′-CH₂—O—N(R)-2′) BNA, oxyamino (4′-CH₂—N(R)—O-2′) BNA, methyl(methyleneoxy) (4′-CH(CH₃)—O-2′) BNA (also referred to as constrained ethyl or cEt), methylene-thio (4′-CH₂—S-2′) BNA, methylene-amino (4′-CH₂—N(R)-2′) BNA, methyl carbocyclic (4′-CH₂—CH(CH₃)-2′) BNA, propylene carbocyclic (4′-(CH₂)₃-2′) BNA, or vinyl BNA.

In some embodiments, a sugar modification is 2′-OMe, 2′-MOE, 2′-LNA, 2′-F, 5′-vinyl, or S-cEt. In some embodiments, a modified sugar is a sugar of FRNA, FANA, or morpholino. In some embodiments, an oligonucleotide comprises a nucleic acid analog, e.g., GNA, LNA, PNA, TNA, F-HNA (F-THP or 3′-fluoro tetrahydropyran), MNA (mannitol nucleic acid, e.g., Leumann 2002 Bioorg. Med. Chem. 10: 841-854), ANA (anitol nucleic acid), or morpholino, or a portion thereof. In some embodiments, a sugar modification replaces a natural sugar with another cyclic or acyclic moiety. Examples of such moieties are widely known in the art, e.g., those used in morpholino, glycol nucleic acids, etc. and may be utilized in accordance with the present disclosure. As appreciated by those skilled in the art, when utilized with modified sugars, in some embodiments internucleotidic linkages may be modified, e.g., as in morpholino, PNA, etc.

In some embodiments, a sugar is a 6′-modified bicyclic sugar that have either (R) or (S)-chirality at the 6-position, e.g., those described in U.S. Pat. No. 7,399,845. In some embodiments, a sugar is a 5′-modified bicyclic sugar that has either (R) or (S)-chirality at the 5-position, e.g., those described in US 20070287831.

In some embodiments, a modified sugar contains one or more substituents at the 2′ position (typically one substituent, and often at the axial position) independently selected from —F; —CF₃, —CN, —N₃, —NO, —NO₂, —OR′, —SR′, or —N(R′)₂, wherein each R′ is independently optionally substituted C₁₋₁₀ aliphatic; —O—(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—(C₁-C₁₀ alkyl), —O—(C₁-C₁₀ alkylene)-NH—(C₁-C₁₀ alkyl) or —O—(C₁-C₁₀ alkylene)-NH(C₁-C₁₀ alkyl)₂, —NH—(C₁-C₁₀ alkylene)-O—(C₁-C₁₀ alkyl), or —N(C₁-C₁₀ alkyl)-(C₁-C₁₀ alkylene)-O—(C₁-C₁₀ alkyl), wherein each of the alkyl, alkylene, alkenyl and alkynyl is independently and optionally substituted. In some embodiments, a substituent is —O(CH₂)·OCH₃, —O(CH₂)ₙNH₂, MOE, DMAOE, or DMAEOE, wherein wherein n is from 1 to about 10. In some embodiments, a modified sugar is one described in WO 2001/088198; and Martin et al., Helv. Chim. Acta, 1995, 78, 486-504. In some embodiments, a modified sugar comprises one or more groups selected from a substituted silyl group, an RNA cleaving group, a reporter group, a fluorescent label, an intercalator, a group for improving the pharmacokinetic properties of a nucleic acid, a group for improving the pharmacodynamic properties of a nucleic acid, or other substituents having similar properties. In some embodiments, modifications are made at one or more of the 2′, 3′, 4′, or 5′ positions, including the 3′ position of the sugar on the 3′-terminal nucleoside or in the 5′ position of the 5′-terminal nucleoside.

In some embodiments, the 2′-OH of a ribose is replaced with a group selected from —H, —F; —CF₃, —CN, —N₃, —NO, —NO₂, —OR′, —SR′, or —N(R′)₂, wherein each R′ is independently described in the present disclosure; —O—(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—(C₁-C₁₀ alkyl), —O—(C₁-C₁₀ alkylene)-NH—(C₁-C₁₀ alkyl) or —O—(C₁-C₁₀ alkylene)-NH(C₁-C₁₀ alkyl)₂, —NH—(C₁-C₁₀ alkylene)-O—(C₁-C₁₀ alkyl), or —N(C₁-C₁₀ alkyl)-(C₁-C₁₀ alkylene)-O—(C₁-C₁₀ alkyl), wherein each of the alkyl, alkylene, alkenyl and alkynyl is independently and optionally substituted. In some embodiments, the 2′-OH is replaced with —H (deoxyribose). In some embodiments, the 2′-OH is replaced with —F. In some embodiments, the 2′-OH is replaced with —OR′. In some embodiments, the 2′-OH is replaced with —OMe. In some embodiments, the 2′-OH is replaced with —OCH₂CH₂OMe.

In some embodiments, a sugar modification is a 2′-modification. Commonly used 2′-modifications include but are not limited to 2′-OR, wherein R is optionally substituted C₁₋₆ aliphatic. In some embodiments, a modification is 2′-OR, wherein R is optionally substituted C₁₋₆ alkyl. In some embodiments, a modification is 2′-OMe. In some embodiments, a modification is 2′-MOE. In some embodiments, a 2′-modification is S-cEt. In some embodiments, a modified sugar is an LNA sugar. In some embodiments, a 2′-modification is —F. In some embodiments, a 2′-modification is FANA. In some embodiments, a 2′-modification is FRNA. In some embodiments, a sugar modification is a 5′-modification, e.g., 5′-Me. In some embodiments, a sugar modification changes the size of the sugar ring. In some embodiments, a sugar modification is the sugar moiety in FHNA. In some embodiments, a 2′-modification is 2′-F.

In some embodiments, a sugar modification replaces a sugar moiety with another cyclic or acyclic moiety. Examples of such moieties are widely known in the art, including but not limited to those used in morpholino (optionally with its phosphorodiamidate linkage), glycol nucleic acids, etc.

In some embodiments, 5% or more of the sugars of an USH2A oligonucleotide are modified. In some embodiments, 10% or more of the sugars of an oligonucleotide are modified. In some embodiments, 15% or more of the sugars of an oligonucleotide are modified. In some embodiments, 20% or more of the sugars of an oligonucleotide are modified. In some embodiments, 25% or more of the sugars of an oligonucleotide are modified. In some embodiments, 30% or more of the sugars of an oligonucleotide are modified. In some embodiments, 35% or more of the sugars of an oligonucleotide are modified. In some embodiments, 40% or more of the sugars of an oligonucleotide are modified. In some embodiments, 45% or more of the sugars of an oligonucleotide are modified. In some embodiments, 50% or more of the sugars of an oligonucleotide are modified. In some embodiments, 55% or more of the sugars of an oligonucleotide are modified. In some embodiments, 60% or more of the sugars of an oligonucleotide are modified. In some embodiments, 65% or more of the sugars of an oligonucleotide are modified. In some embodiments, 70% or more of the sugars of an oligonucleotide are modified. In some embodiments, 75% or more of the sugars of an oligonucleotide are modified. In some embodiments, 80% or more of the sugars of an oligonucleotide are modified. In some embodiments, 85% or more of the sugars of an oligonucleotide are modified. In some embodiments, 90% or more of the sugars of an oligonucleotide are modified. In some embodiments, 95% or more of the sugars of an oligonucleotide are modified. In some embodiments, each sugar of an oligonucleotide is independently modified. In some embodiments, a modified sugar comprises a 2′-modification. In some embodiments, each modified sugar independently comprises a 2′-modification. In some embodiments, a 2′-modification is 2′-OR¹. In some embodiments, a 2′-modification is a 2′-OMe. In some embodiments, a 2′-modification is a 2′-MOE. In some embodiments, a 2′-modification is an LNA sugar modification. In some embodiments, a 2′-modification is 2′-F. In some embodiments, each sugar modification is independently a 2′-modification. In some embodiments, each sugar modification is independently 2′-OR¹ or 2′-F. In some embodiments, each sugar modification is independently 2′-OR¹ or 2′-F, wherein R¹ is optionally substituted C₁₋₆ alkyl. In some embodiments, each sugar modification is independently 2′-OR¹ or 2′-F, wherein at least one is 2′-F. In some embodiments, each sugar modification is independently 2′-OR¹ or 2′-F, wherein R¹ is optionally substituted C₁₋₆ alkyl, and wherein at least one is 2′-OR¹. In some embodiments, each sugar modification is independently 2‘—OR’ or 2′-F, wherein at least one is 2′-F, and at least one is 2′-OR¹. In some embodiments, each sugar modification is independently 2′-OR¹ or 2′-F, wherein R¹ is optionally substituted C₁₋₆ alkyl, and wherein at least one is 2′-F, and at least one is 2′-OR¹. In some embodiments, each sugar modification is independently 2′-OR¹. In some embodiments, each sugar modification is independently 2′-OR¹, wherein R¹ is optionally substituted C₁₋₆ alkyl. In some embodiments, each sugar modification is 2′-OMe. In some embodiments, each sugar modification is 2′-MOE. In some embodiments, each sugar modification is independently 2′-OMe or 2′-MOE. In some embodiments, each sugar modification is independently 2′-OMe, 2′-MOE, or a LNA sugar.

In some embodiments, each sugar independently comprises a 2′-F or 2′-OR modification, wherein R is independently C₁₋₆ aliphatic. In some embodiments, R is —CH₃.

In some embodiments, one or more (1-50, 1-40, 1-30, 1-25, 1-20, e.g., 1, 2, 3, 4, 5, 6, 7, 8, 9, 10, 11, 12, 13, 14, 15, 16, 17, 18, 19, 20, 21, 22, 23, 24, or 25, or more) sugars in an oligonucleotide comprise 2′-F modification. In some embodiments, at least 10%, 20%, 30%, 40%, 5%, 60%, 70%, 75%, 80%, 85%, 90%, or 95%, or 100% of all sugars in an oligonucleotide comprise a 2′-F modification. In some embodiments, an oligonucleotide is or comprises a structure of 5′-a first region-a second region-a third region. In some embodiments, each of the regions independently comprises one or more (1-50, 1-40, 1-30, 1-25, 1-20, e.g., 1, 2, 3, 4, 5, 6, 7, 8, 9, 10, 11, 12, 13, 14, 15, 16, 17, 18, 19, 20, 21, 22, 23, 24, or 25, or more) sugars comprises 2′-F modification. In some embodiments, at least 10%, 20%, 30%, 40%, 50%, 60%, 70%, 75%, 80%, 85%, 90%, or 95%, or 100% of all sugars in each of the regions independently comprise a 2′-F modification. In some embodiments, the number of 2′-F modified sugars in an oligonucleotide or a region is 2 or more. In some embodiments, it is 3 or more. In some embodiments, it is 4 or more. In some embodiments, it is 5 or more. In some embodiments, it is 6 or more. In some embodiments, it is 7 or more. In some embodiments, it is 8 or more. In some embodiments, it is 9 or more. In some embodiments, it is 10 or more. In some embodiments, the percentage of 2′-F modified sugars in an oligonucleotide or a region is 50% or more. In some embodiments, it is 60% or more. In some embodiments, it is 70% or more. In some embodiments, it is 80% or more. In some embodiments, it is 90% or more. In some embodiments, it is 95% or more. In some embodiments, it is 100%. In some embodiments, two or more or all 2′-F modified sugars are consecutive.

In some embodiments, a first region comprises 1, 2, 3, 4, 5, 6, 7, 8, 9, or more 2′-F modified sugars. In some embodiments, a first region comprises 5, 6, 7, or 8 2′-F modified sugars. In some embodiments, 50%, 60%, 70%, 75%, 80%, 85%, 90%, or 95%, or 100% of all sugars in a first region comprise 2′-F. In some embodiments, each sugar is a first region comprises 2′-F. In some embodiments, a first region comprises one or more (e.g., 1, 2, 3, 4, 5, 6, 7, 8, 9, 10 or more; in some embodiments, 5 or more) phosphorothioate internucleotidic linkages. In some embodiments, each phosphorothioate internucleotidic linkage in a first region is independently chirally controlled and is Sp. In some embodiments, a first region comprises one or more (e.g., 1, 2, 3, 4, 5, 6, 7, 8, 9, 10 or more) non-negatively charged internucleotidic linkages. In some embodiments, each non-negatively charged internucleotidic linkage in a first region is chirally controlled. In some embodiments, one or more non-negatively charged internucleotidic linkage in a first region is not chirally controlled. In some embodiments, each non-negatively charged internucleotidic linkage in a first region is chirally controlled and is Rp. In some embodiments, two or more or all 2′-F modified sugars in a first region are consecutive.

In some embodiments, a second region comprises 1, 2, 3, 4, 5, 6, 7, 8, 9, or more 2′-F modified sugars. In some embodiments, a second region comprises 5, 6, 7, or 8 2′-F modified sugars. In some embodiments, 50%, 60%, 70%, 75%, 80%, 85%, 90%, or 95%, or 100% of all sugars in a second region comprise 2′-F. In some embodiments, each sugar is a second region comprises 2′-F. In some embodiments, a second region comprises one or more (e.g., 1, 2, 3, 4, 5, 6, 7, 8, 9, 10 or more; in some embodiments, 5 or more) phosphorothioate internucleotidic linkages. In some embodiments, each phosphorothioate internucleotidic linkage in a second region is independently chirally controlled and is Sp. In some embodiments, a second region comprises one or more (e.g., 1, 2, 3, 4, 5, 6, 7, 8, 9, 10 or more) non-negatively charged internucleotidic linkages. In some embodiments, each non-negatively charged internucleotidic linkage in a second region is chirally controlled. In some embodiments, one or more non-negatively charged internucleotidic linkage in a second region is not chirally controlled. In some embodiments, each non-negatively charged internucleotidic linkage in a second region is chirally controlled and is Rp. In some embodiments, each internucleotidic linkage in a second region is independently a phosphorothioate internucleotidic linkage. In some embodiments, two or more or all 2′-F modified sugars in a second region are consecutive. In some embodiments, a second region comprises one or more (e.g., 1, 2, 3, 4, 5, 6, 7, 8, 9, 10 or more) sugars that are not 2′-F modified. In some embodiments, one or more (e.g., 1, 2, 3, 4, 5, 6, 7, 8, 9, 10 or more) or all sugars that are not 2′-F modified are 2′-OR modified, wherein R is optionally substituted C₁₋₆ aliphatic. In some embodiments, a second region comprises alternating 2′-F modified sugars and 2′-OR modified sugars, wherein R is optionally substituted C₁₋₆ aliphatic. In some embodiments, the first sugar in a second region (from 5′ to 3′) is a 2′-OR modified sugar, wherein R is optionally substituted C₁₋₆ aliphatic. In some embodiments, the last sugar in a second region (from 5′ to 3′) is a 2′-OR modified sugar, wherein R is optionally substituted C₁₋₆ aliphatic. In some embodiments, both the first and last sugars in a second region are independently a 2′-OR modified sugar, wherein R is optionally substituted C₁₋₆ aliphatic. In some embodiments, R is methyl.

In some embodiments, a third region comprises 1, 2, 3, 4, 5, 6, 7, 8, 9, or more 2′-F modified sugars. In some embodiments, a third region comprises 5, 6, 7, or 8 2′-F modified sugars. In some embodiments, 50%, 60%, 70%, 75%, 80%, 85%, 90%, or 95%, or 100% of all sugars in a third region comprise 2′-F. In some embodiments, each sugar is a third region comprises 2′-F. In some embodiments, a third region comprises one or more (e.g., 1, 2, 3, 4, 5, 6, 7, 8, 9, 10 or more; in some embodiments, 5 or more) phosphorothioate internucleotidic linkages. In some embodiments, each phosphorothioate internucleotidic linkage in a third region is independently chirally controlled and is Sp. In some embodiments, a third region comprises one or more (e.g., 1, 2, 3, 4, 5, 6, 7, 8, 9, 10 or more) non-negatively charged internucleotidic linkages. In some embodiments, each non-negatively charged internucleotidic linkage in a third region is chirally controlled. In some embodiments, one or more non-negatively charged internucleotidic linkage in a third region is not chirally controlled. In some embodiments, each non-negatively charged internucleotidic linkage in a third region is chirally controlled and is Rp. In some embodiments, two or more or all 2′-F modified sugars in a third region are consecutive.

In some embodiments, one or more (1-50, 1-40, 1-30, 1-25, 1-20, e.g., 1, 2, 3, 4, 5, 6, 7, 8, 9, 10, 11, 12, 13, 14, 15, 16, 17, 18, 19, 20, 21, 22, 23, 24, or 25, or more) sugars comprises 2′-F modification.

In some embodiments, sugars are connected by internucleotidic linkages, in some embodiments, modified internucleotidic linkage. In some embodiments, an internucleotidic linkage does not contain a linkage phosphorus. In some embodiments, an internucleotidic linkage is -L-. In some embodiments, an internucleotidic linkage is —OP(O)(—C≡CH)O—, —OP(O)(R)O— (e.g., R is —CH₃), 3′ —NHP(O)(OH)O—5′, 3′-OP(O)(CH₃)OCH₂-5′, 3′-CH₂C(O)NHCH₂-5′, 3′-SCH₂OCH₂-5′, 3′-OCH₂OCH₂-5′, 3′-CH₂NR′CH₂-5′, 3′-CH₂N(Me)OCH₂-5′, 3′-NHC(O)CH₂CH₂-5′, 3′-NR′C(O)CH₂CH₂-5′, 3′-CH₂CH₂NR′-5′, 3′-CH₂CH₂NH-5′, or 3′-OCH₂CH₂N(R′)-5′. In some embodiments, a 5′ carbon may be optionally substituted with ═O.

In some embodiments, a modified sugar is an optionally substituted pentose or hexose. In some embodiments, a modified sugar is an optionally substituted pentose. In some embodiments, a modified sugar is an optionally substituted hexose. In some embodiments, a modified sugar is an optionally substituted ribose or hexitol. In some embodiments, a modified sugar is an optionally substituted ribose. In some embodiments, a modified sugar is an optionally substituted hexitol.

In some embodiments, a sugar modification is 5′-vinyl (R or S), 5′-methyl (R or S), 2′-SH, 2′-F, 2′-OCH₃, 2′-OCH₂CH₃, 2′-OCH₂CH₂F or 2′-O(CH₂)₂₀CH₃. In some embodiments, a substituent at the 2′ position, e.g., a 2′-modification, is allyl, amino, azido, thio, O-allyl, O—C₁-C₁₀ alkyl, OCF₃, OCH₂F, O(CH₂)₂SCH₃, O(CH₂)₂—O—N(Rₘ)(Rₙ), O—CH₂—C(═O)—N(Rₘ)(Rₙ), and O—CH₂—C(═O)—N(Rₗ)—(CH₂)₂—N(Rₘ)(Rₙ), wherein each allyl, amino and alkyl is optionally substituted, and each of Rₗ, Rₘ and Rₙ is independently R′ as described in the present disclosure. In some embodiments, each of Rₗ, Rₘ and Rₙ is independently —H or optionally substituted C₁-C₁₀ alkyl.

In some embodiments, a sugar is a tetrahydropyran or THP sugar. In some embodiments, a modified nucleoside is tetrahydropyran nucleoside or THP nucleoside which is a nucleoside having a six-membered tetrahydropyran sugar substituted for a pentofuranosyl residue in typical natural nucleosides. THP sugars and/or nucleosides include those used in hexitol nucleic acid (HNA), anitol nucleic acid (ANA), mannitol nucleic acid (MNA) (e.g., Leumann, Bioorg. Med. Chem., 2002, 10, 841-854) or fluoro HNA (F-HNA).

In some embodiments, sugars comprise rings having more than 5 atoms and/or more than one heteroatom, e.g., morpholino sugars.

As those skilled in the art will appreciate, modifications of sugars, nucleobases, internucleotidic linkages, etc. can and are often utilized in combination in oligonucleotides, e.g., see various oligonucleotides in Table A1. For example, a combination of sugar modification and nucleobase modification is 2′-F (sugar) 5-methyl (nucleobase) modified nucleosides. In some embodiments, a combination is replacement of a ribosyl ring oxygen atom with S and substitution at the 2′-position.

In some embodiments, a 2′-modified sugar is a furanosyl sugar modified at the 2′ position. In some embodiments, a 2′-modification is halogen, —R′ (wherein R′ is not —H), —OR′ (wherein R′ is not —H), —SR′, —N(R′)₂, optionally substituted —CH₂—CH═CH₂, optionally substituted alkenyl, or optionally substituted alkynyl. In some embodiments, a 2′-modifications is selected from —O[(CH₂)ₙO]ₘCH₃, —O(CH₂)ₙNH₂, —O(CH₂)ₙCH₃, —O(CH₂)ₙF, —O(CH₂)ₙONH₂, —OCH₂C(═O)N(H)CH₃, and —O(CH₂)ₙON[(CH₂)ₙCH₃]₂, wherein each n and m is independently from 1 to about 10. In some embodiments, a 2′-modification is optionally substituted C₁-C₁₂ alkyl, optionally substituted alkenyl, optionally substituted alkynyl, optionally substituted alkaryl, optionally substituted aralkyl, optionally substituted —O-alkaryl, optionally substituted —O-aralkyl, —SH, —SCH₃, —OCN, —Cl, —Br, —CN, —F, —CF₃, —OCF₃, —SOCH₃, —SO₂CH₃, —ONO₂, —NO₂, —N₃, —NH₂, optionally substituted heterocycloalkyl, optionally substituted heterocycloalkaryl, optionally substituted aminoalkylamino, optionally substituted polyalkylamino, substituted silyl, a reporter group, an intercalator, a group for improving pharmacokinetic properties, a group for improving the pharmacodynamic properties, and other substituents. In some embodiments, a 2′-modification is a 2′-MOE modification.

In some embodiments, a 2′-modified or 2′-substituted sugar or nucleoside is a sugar or nucleoside comprising a substituent at the 2′ position of the sugar which is other than —H (typically not considered a substituent) or —OH. In some embodiments, a 2′-modified sugar is a bicyclic sugar comprising a bridge connecting two carbon atoms of the sugar ring one of which is the 2′ carbon. In some embodiments, a 2′-modification is non-bridging, e.g., allyl, amino, azido, thio, optionally substituted —O-allyl, optionally substituted —O—C₁-C₁₀ alkyl, —OCF₃, —O(CH₂)₂OCH₃, 2′-O(CH₂)₂SCH₃, —O(CH₂)₂ON(Rₘ)(Rₙ), or —OCH₂C(═O)N(Rₘ)(Rₙ), where each Rₘ and Rₙ is independently —H or optionally substituted C₁-C₁₀ alkyl.

In some embodiments, a sugar is the sugar of N-methanocarba, LNA, cMOE BNA, cEt BNA, α-L-LNA or related analogs, HNA, Me-ANA, MOE-ANA, Ara-FHNA, FHNA, R-6′-Me-FHNA, S-6′-Me-FHNA, ENA, or c-ANA. In some embodiments, a modified internucleotidic linkage is C3-amide (e.g., sugar that has the amide modification attached to the C3′, Mutisya et al. 2014 Nucleic Acids Res. 2014 Jun. 1; 42(10): 6542-6551), formacetal, thioformacetal, MMI [e.g., methylene(methylimino), Peoc'h et al. 2006 Nucleosides and Nucleotides 16 (7-9)], a PMO (phosphorodiamidate linked morpholino) linkage (which connects two sugars), or a PNA (peptide nucleic acid) linkage.

In some embodiments, a sugar is one described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612, the sugars of each of which is incorporated herein by reference.

Various additional sugars useful for preparing oligonucleotides or analogs thereof are known in the art and may be utilized in accordance with the present disclosure.

In some embodiments, an USH2A oligonucleotide can comprise any sugar described herein or known in the art. In some embodiments, an USH2A oligonucleotide can comprise any sugar described herein or known in the art in combination with any other structural element or modification described herein, including but not limited to, base sequence or portion thereof, base; internucleotidic linkage; stereochemistry or pattern thereof, additional chemical moiety, including but not limited to, a targeting moiety, etc.; pattern of modifications of sugars, bases or internucleotidic linkages; format or any structural element thereof, and/or any other structural element or modification described herein; and in some embodiments, the present disclosure pertains to multimers of any such oligonucleotides.

### Nucleobases

Various nucleobases may be utilized in provided oligonucleotides in accordance with the present disclosure. In some embodiments, a nucleobase is a natural nucleobase, the most commonly occurring ones being A, T, C, G and U. In some embodiments, a nucleobase is a modified nucleobase in that it is not A, T, C, G or U. In some embodiments, a nucleobase is optionally substituted A, T, C, G or U, or a substituted tautomer of A T, C, G or U. In some embodiments, a nucleobase is optionally substituted A, T, C, G or U, e.g., 5mC, 5-hydroxymethyl C, etc. In some embodiments, a nucleobase is alkyl-substituted A, T, C, G or U. In some embodiments, a nucleobase is A. In some embodiments, a nucleobase is T. In some embodiments, a nucleobase is C. In some embodiments, a nucleobase is G. In some embodiments, a nucleobase is U. In some embodiments, a nucleobase is 5mC. In some embodiments, a nucleobase is substituted A, T, C, G or U. In some embodiments, a nucleobase is a substituted tautomer of A, T, C, G or U. In some embodiments, substitution protects certain functional groups in nucleobases to minimize undesired reactions during oligonucleotide synthesis. Suitable technologies for nucleobase protection in oligonucleotide synthesis are widely known in the art and may be utilized in accordance with the present disclosure. In some embodiments, modified nucleobases improves properties and/or activities of oligonucleotides. For example, in many cases, 5mC may be utilized in place of C to modulate certain undesired biological effects, e.g., immune responses. In some embodiments, when determining sequence identity, a substituted nucleobase having the same hydrogen-bonding pattern is treated as the same as the unsubstituted nucleobase, e.g., 5mC may be treated the same as C [e.g., an oligonucleotide having 5mC in place of C (e.g., AT5mCG) is considered to have the same base sequence as an oligonucleotide having C at the corresponding location(s) (e.g., ATCG)].

In some embodiments, an oligonucleotide comprises one or more A, T, C, G or U. In some embodiments, an oligonucleotide comprises one or more optionally substituted A, T, C, G or U. In some embodiments, an oligonucleotide comprises one or more 5-methylcytidine, 5-hydroxymethylcytidine, 5-formylcytosine, or 5-carboxylcytosine. In some embodiments, an oligonucleotide comprises one or more 5-methylcytidine. In some embodiments, each nucleobase in an oligonucleotide is selected from the group consisting of optionally substituted A, T, C, G and U, and optionally substituted tautomers of A, T, C, G and U. In some embodiments, each nucleobase in an oligonucleotide is optionally protected A, T, C, G and U. In some embodiments, each nucleobase in an oligonucleotide is optionally substituted A, T, C, G or U. In some embodiments, each nucleobase in an oligonucleotide is selected from the group consisting of A, T, C, G, U, and 5mC.

In some embodiments, a nucleobase is a natural nucleobase or a modified nucleobase derived from a natural nucleobase. Examples include uracil, thymine, adenine, cytosine, and guanine optionally having their respective amino groups protected by acyl protecting groups, 2-fluorouracil, 2-fluorocytosine, 5-bromouracil, 5-iodouracil, 2,6-diaminopurine, azacytosine, pyrimidine analogs such as pseudoisocytosine and pseudouracil and other modified nucleobases such as 8-substituted purines, xanthine, or hypoxanthine (the latter two being the natural degradation products). Certain examples of modified nucleobases are disclosed in Chiu and Rana, R N A, 2003, 9, 1034-1048, Limbach et al. Nucleic Acids Research, 1994, 22, 2183-2196 and Revankar and Rao, Comprehensive Natural Products Chemistry, vol. 7, 313. In some embodiments, a modified nucleobase is substituted uracil, thymine, adenine, cytosine, or guanine. In some embodiments, a modified nucleobase is a functional replacement, e.g., in terms of hydrogen bonding and/or base pairing, of uracil, thymine, adenine, cytosine, or guanine. In some embodiments, a nucleobase is optionally substituted uracil, thymine, adenine, cytosine, 5-methylcytosine, or guanine. In some embodiments, a nucleobase is uracil, thymine, adenine, cytosine, 5-methylcytosine, or guanine.

In some embodiments, a provided oligonucleotide comprises one or more 5-methylcytosine. In some embodiments, the present disclosure provides an oligonucleotide whose base sequence is disclosed herein, e.g., in Table A1, wherein each T may be independently replaced with U and vice versa, and each cytosine is optionally and independently replaced with 5-methylcytosine or vice versa. As appreciated by those skilled in the art, in some embodiments, 5mC may be treated as C with respect to base sequence of an oligonucleotide—such oligonucleotide comprises a nucleobase modification at the C position (e.g., see various oligonucleotides in Table A1). In description of oligonucleotides, typically unless otherwise noted, nucleobases, sugars and internucleotidic linkages are non-modified, or are modified as indicated. For example, in WV-24366 (5′-fU*SfG*SfAn001fG*SfG*SfAn001fU*SfU*SmGfC*SmA*SfG*SmAfA*SfU*SfU*SfUn001fG*SfU*SfU-3′) and WV-24360 (5′-fG*SfG*SfA*SfU*SfU*SfG*SfC*SfA*SmGfA*SmA*SfU*SmUfU*SfG*SfU*SfU*SfC*SfA*SfC-3′), fU, fG, fA, etc., are modified as indicated (U, G, A, etc., which are each 2′-F modified); mA, mG, mU, etc., are modified as indicated (A, G, U, etc., which are each 2′-OMe modified); and each internucleotidic linkage, unless otherwise noted, is independently a natural phosphate linkage (e.g., natural phosphate linkages between mU and fU in . . . *SfU*SmUfU*SfG* . . . in WV-24360); and each Sp phosphorothioate internucleotidic linkage is represented by * S (or *S); and each neutral or non-negatively charged internucleotidic linkage is indicated by n001.

In some embodiments, a modified base is optionally substituted adenine, cytosine, guanine, thymine, or uracil, or a tautomer thereof. In some embodiments, a modified nucleobase is a modified adenine, cytosine, guanine, thymine or uracil, modified by one or more modifications by which:
(1) a nucleobase is modified by one or more optionally substituted groups independently selected from acyl, halogen, amino, azide, alkyl, alkenyl, alkynyl, aryl, heteroalkyl, heteroalkenyl, heteroalkynyl, heterocyclyl, heteroaryl, carboxyl, hydroxyl, biotin, avidin, streptavidin, substituted silyl, and combinations thereof,
(2) one or more atoms of a nucleobase are independently replaced with a different atom selected from carbon, nitrogen and sulfur;
(3) one or more double bonds in a nucleobase are independently hydrogenated; or
(4) one or more aryl or heteroaryl rings are independently inserted into a nucleobase.

In some embodiments, a modified nucleobase is a modified nucleobase known in the art, e.g., WO2017/210647. In some embodiments, modified nucleobases are expanded-size nucleobases in which one or more aryl and/or heteroaryl rings, such as phenyl rings, have been added.

In some embodiments, a modified nucleobase is selected from 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. In some embodiments, modified nucleobases are tricyclic pyrimidines, such as 1,3-diazaphenoxazine-2-one, 1,3-diazaphenothiazine-2-one or 9-(2-aminoethoxy)-1,3-diazaphenoxazine-2-one (G-clamp). In some embodiments, modified nucleobases are those in which the purine or pyrimidine base is replaced with other heterocycles, for example, 7-deaza-adenine, 7-deazaguanosine, 2-aminopyridine or 2-pyridone.

In some embodiments, a modified nucleobase is substituted. In some embodiments, a modified nucleobase is substituted such that it contains, e.g., heteroatoms, alkyl groups, or linking moieties connected to fluorescent moieties, biotin or avidin moieties, or other protein or peptides. In some embodiments, a modified nucleobase is a “universal base” that is not a nucleobase in the most classical sense, but that functions similarly to a nucleobase. One example of a universal base is 3-nitropyrrole.

In some embodiments, nucleosides that can be utilized in provided technologies comprise modified nucleobases and/or modified sugars, e.g., 4-acetylcytidine; 5-(carboxyhydroxylmethyl)uridine; 2′-O-methylcytidine; 5-carboxymethylaminomethyl-2-thiouridine; 5-carboxymethylaminomethyluridine; dihydrouridine; 2′-O-methylpseudouridine; beta,D-galactosylqueosine; 2′-O-methylguanosine; N⁶-isopentenyladenosine; 1-methyladenosine; 1-methylpseudouridine; 1-methylguanosine; 1-methylinosine; 2,2-dimethylguanosine; 2-methyladenosine; 2-methylguanosine; N⁷-methylguanosine; 3-methyl-cytidine; 5-methylcytidine; 5-hydroxymethylcytidine; 5-formylcytosine; 5-carboxylcytosine; N⁶-methyladenosine; 7-methylguanosine; 5-methylaminoethyluridine; 5-methoxyaminomethyl-2-thiouridine; beta,D-mannosylqueosine; 5-methoxycarbonylmethyluridine; 5-methoxyuridine; 2-methylthio-N⁶-isopentenyladenosine; N-((9-beta,D-ribofuranosyl-2-methylthiopurine-6-yl)carbamoyl)threonine; N-((9-beta,D-ribofuranosylpurine-6-yl)-N-methylcarbamoyl)threonine; uridine-5-oxyacetic acid methylester; uridine-5-oxyacetic acid (v); pseudouridine; queosine; 2-thiocytidine; 5-methyl-2-thiouridine; 2-thiouridine; 4-thiouridine; 5-methyluridine; 2′-O-methyl-5-methyluridine; and 2′-O-methyluridine.

In some embodiments, a nucleobase, e.g., a modified nucleobase comprises one or more biomolecule binding moieties such as e.g., antibodies, antibody fragments, biotin, avidin, streptavidin, receptor ligands, or chelating moieties. In other embodiments, a nucleobase is 5-bromouracil, 5-iodouracil, or 2,6-diaminopurine. In some embodiments, a nucleobase comprises substitution with a fluorescent or biomolecule binding moiety. In some embodiments, a substituent is a fluorescent moiety. In some embodiments, a substituent is biotin or avidin.

In some embodiments, a nucleobase is one described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612, the nucleobases of each of which is incorporated herein by reference.

### Additional Chemical Moieties

In some embodiments, an USH2A oligonucleotide comprises one or more additional chemical moieties. Various additional chemical moieties, e.g., targeting moieties, carbohydrate moieties, lipid moieties, etc. are known in the art and can be utilized in accordance with the present disclosure to modulate properties and/or activities of USH2A oligonucleotides, e.g., stability, half life, activities, delivery, pharmacodynamics properties, pharmacokinetic properties, etc. In some embodiments, certain additional chemical moieties facilitate delivery of oligonucleotides to desired cells, tissues and/or organs, including but not limited the cells of the eye and/or ear (e.g., retinal cells and/or cochlear cells) and/or any other tissue or organ which expresses USH2A. In some embodiments, certain additional chemical moieties facilitate internalization of oligonucleotides. In some embodiments, certain additional chemical moieties increase oligonucleotide stability. In some embodiments, the present disclosure provides technologies for incorporating various additional chemical moieties into oligonucleotides.

In some embodiments, an USH2A oligonucleotide comprises an additional chemical moiety demonstrates increased delivery to and/or activity in a tissue or an organ (e.g., eye or a part thereof) compared to a reference oligonucleotide, e.g., a reference oligonucleotide which does not have the additional chemical moiety but is otherwise identical.

In some embodiments, additional chemical moieties are carbohydrate moieties, targeting moieties, etc., which, when incorporated into oligonucleotides, can improve one or more properties. In some embodiments, an additional chemical moiety is selected from glucose, GluNAc (N-acetyl amine glucosamine) and anisamide moieties.

In some embodiments, an additional chemical moiety is a targeting moiety. In some embodiments, an additional chemical moiety is or comprises a carbohydrate moiety. In some embodiments, an additional chemical moiety is or comprises a lipid moiety. In some embodiments, an additional chemical moiety is or comprises a ligand moiety for, e.g., cell receptors such as a sigma receptor, an asialoglycoprotein receptor, etc. In some embodiments, a ligand moiety is or comprises an anisamide moiety, which may be a ligand moiety for a sigma receptor. In some embodiments, an additional chemical moiety is or comprises a ligand moiety for an asialoglycoprotein receptor.

Certain useful additional chemical moieties are described in U.S. Ser. No. 10/479,995, US 2020/0056173, US 2018/0216107, US 2019/0127733, U.S. Pat. No. 10,450,568, US 2019/0249173, WO 2018/223056, WO 2018/223073, WO 2018/223081, WO 2019/032607, WO 2019/075357, WO 2019/200185, WO 2019/217784, and/or WO 2019/032612.

### Production of Oligonucleotides and Compositions

Various methods can be utilized for production of oligonucleotides and compositions and can be utilized in accordance with the present disclosure. For example, traditional phosphoramidite chemistry can be utilized to prepare stereorandom oligonucleotides and compositions, and certain reagents and chirally controlled technologies can be utilized to prepare chirally controlled oligonucleotide compositions, e.g., as described in U.S. Pat. Nos. 9,394,333, 9,744,183, 9,605,019, 9,598,458, 9,982,257, 10,160,969, 10/479,995, US 2020/0056173, US 2018/0216107, US 2019/0127733, U.S. Pat. No. 10,450,568, US 2019/0077817, US 2019/0249173, US 2019/0375774, a WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612, the reagents and methods of each of which is incorporated herein by reference.

In some embodiments, chirally controlled/stereoselective preparation of oligonucleotides and compositions thereof comprise utilization of a chiral auxiliary, e.g., as part of monomeric phosphoramidites. Examples of such chiral auxiliary reagents and phosphoramidites are described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612, the chiral auxiliary reagents and phosphoramidites of each of which are independently incorporated herein by reference. In some embodiments, a chiral auxiliary is

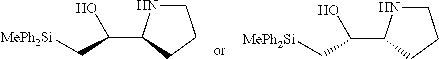

(DPSE chiral auxiliaries). In some embodiments, a chiral auxiliary is

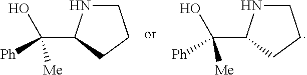 [SMILES: C[C@@](O)(c1ccccc1)[C@@H]1CCCN1 ; C[C@](O)(c1ccccc1)[C@H]1CCCN1]

In some embodiments, a chiral auxiliary is

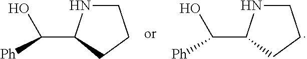 [SMILES: O[C@H](c1ccccc1)[C@@H]1CCCN1 ; O[C@@H](c1ccccc1)[C@H]1CCCN1]

In some embodiments, a chiral auxiliary comprises —SO₂R^{AU}, wherein R^{AU} is an optionally substituted group selected from C₁₋₂₀ aliphatic, C₁₋₂₀ heteroaliphatic having 1-10 heteroatoms, C₆₋₂₀ aryl, C₆₋₂₀ arylaliphatic, C₆₋₂₀ arylheteroaliphatic having 1-10 heteroatoms, 5-20 membered heteroaryl having 1-10 heteroatoms, and 3-20 membered heterocyclyl having 1-10 heteroatoms. In some embodiments, a chiral auxiliary is

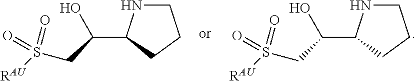

In some embodiments, R^{AU} is optionally substituted aryl. In some embodiments, R^{AU} is optionally substituted phenyl. In some embodiments, R^{AU} is optionally substituted C₁₋₆ aliphatic. In some embodiments, a chiral auxiliary is

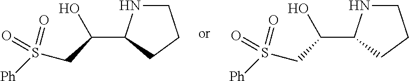 [SMILES: O=S(=O)(C[C@@H](O)[C@@H]1CCCN1)c1ccccc1 ; O=S(=O)(C[C@H](O)[C@H]1CCCN1)c1ccccc1]

(PSM chiral auxiliaries). In some embodiments, utilization of such chiral auxiliaries, e.g., preparation, phosphoramidites comprising such chiral auxiliaries, intermediate oligonucleotides comprising such auxiliaries, protection, removal, etc., is described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612 and incorporated herein by reference.

In some embodiments, chirally controlled preparation technologies, including oligonucleotide synthesis cycles, reagents and conditions are described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612, the oligonucleotide synthesis methods, cycles, reagents and conditions of each of which are independently incorporated herein by reference.

Once synthesized, USH2A oligonucleotides and compositions are typically further purified. Suitable purification technologies are widely known and practiced by those skilled in the art, including but not limited to those described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612, the purification technologies of each of which are independently incorporated herein by reference.

In some embodiments, a cycle comprises or consists of coupling, capping, modification and deblocking. In some embodiments, a cycle comprises or consists of coupling, capping, modification, capping and deblocking. These steps are typically performed in the order they are listed, but in some embodiments, as appreciated by those skilled in the art, the order of certain steps, e.g., capping and modification, may be altered. If desired, one or more steps may be repeated to improve conversion, yield and/or purity as those skilled in the art often perform in syntheses. For example, in some embodiments, coupling may be repeated; in some embodiments, modification (e.g., oxidation to install ═O, sulfurization to install ═S, etc.) may be repeated; in some embodiments, coupling is repeated after modification which can convert a P(III) linkage to a P(V) linkage which can be more stable under certain circumstances, and coupling is routinely followed by modification to convert newly formed P(III) linkages to P(V) linkages. In some embodiments, when steps are repeated, different conditions may be employed (e.g., concentration, temperature, reagent, time, etc.).

In some embodiments, oligonucleotides are linked to a solid support. In some embodiments, a solid support is a support for oligonucleotide synthesis. In some embodiments, a solid support comprises glass. In some embodiments, a solid support is CPG (controlled pore glass). In some embodiments, a solid support is polymer. In some embodiments, a solid support is polystyrene. In some embodiments, the solid support is Highly Crosslinked Polystyrene (HCP). In some embodiments, the solid support is hybrid support of Controlled Pore Glass (CPG) and Highly Cross-linked Polystyrene (HCP). In some embodiments, a solid support is a metal foam. In some embodiments, a solid support is a resin. In some embodiments, oligonucleotides are cleaved from a solid support.

Technologies for formulating provided oligonucleotides and/or preparing pharmaceutical compositions, e.g., for administration to subjects via various routes, are readily available in the art and can be utilized in accordance with the present disclosure, e.g., those described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612.

### Biological Applications

As appreciated by those skilled in the art, USH2A oligonucleotides are useful for multiple purposes. In some embodiments, provided technologies (e.g., USH2A oligonucleotides, compositions, methods, etc.) are useful for mediating skipping of a deleterious exon in an USH2A gene transcript. In some embodiments, provided oligonucleotides and compositions provide improved skipping of a deleterious exon in an USH2A gene transcript, compared to a reference condition selected from the group consisting of absence of the oligonucleotide or composition, presence of a reference oligonucleotide or composition, and combinations thereof. Certain example applications and/or methods for using and making various oligonucleotides are described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, or WO 2018/237194.

For example, in some embodiments, a provided oligonucleotide is an USH2A oligonucleotide capable of mediating an increase in the level of skipping of a deleterious exon in an USH2A gene product. An improvement mediated by an USH2A oligonucleotide can be an improvement of any desired biological functions, including but not limited to treatment and/or prevention of an USH2A-related disorder or a symptom thereof.

In some embodiments, a provided compound, e.g., USH2A oligonucleotide, and/or compositions thereof, can modulate activities and/or functions of an USH2A target gene. In some embodiments, a target gene is an USH2A gene with respect to which expression and/or activity of one or more gene products (e.g., RNA and/or protein products) are intended to be altered. Thus, when an oligonucleotide as described herein acts on a particular target gene, presence and/or activity of one or more gene products of that gene are altered when the oligonucleotide is present as compared with when it is absent. In some embodiments, a target gene is USH2A.

In some embodiments, provided oligonucleotides and compositions are useful for treating various conditions, disorders or diseases, by reducing levels and/or activities of transcripts and/or products encoded thereby that are associated with the conditions, disorders or diseases. In some embodiments, the present disclosure provides methods for preventing or treating a condition, disorder or disease, comprising administering to a subject susceptible to or suffering from a condition, disorder or disease a provided oligonucleotide or composition thereof. In some embodiments, a provided oligonucleotide or oligonucleotides in a provided composition are of a base sequence that is or is complementary to a portion of a transcript, which transcript is associated with a condition, disorder or disease. In some embodiments, a base sequence is such that it selectively bind to a transcript, e.g., an USH2A transcript, associated with a condition, disorder or disease over other transcripts that are not associated with the same condition, disorder or disease. In some embodiments, a condition, disorder or disease is associated with USH2A.

In some embodiments, in a method of treating a disease by administering a composition comprising a plurality of USH2A oligonucleotides sharing a common base sequence, which base sequence is complementary to a target sequence in a target transcript, the present disclosure provides an improvement that comprises administering as the oligonucleotide composition a chirally controlled oligonucleotide composition as described in the present disclosure, characterized in that, when it is contacted with the target transcript in a splicing system, skipping of a deleterious exon in an USH2A gene transcript is improved relative to that observed under a reference condition selected from the group consisting of absence of the composition, presence of a reference composition, and combinations thereof. In some embodiments, a reference composition is a racemic preparation of oligonucleotides of the same sequence or constitution. In some embodiments, a target transcript is an USH2A transcript.

In some embodiments, provided oligonucleotides can bind to a transcript, and improve skipping of a deleterious exon in an USH2A gene transcript. In some embodiments, provided oligonucleotides, e.g., USH2A oligonucleotides, improved skipping of a deleterious exon in an USH2A gene transcript, with efficiency greater than a comparable oligonucleotide under one or more suitable conditions.

In some embodiments, improved skipping of a deleterious exon in an USH2A gene transcript, is at least 10%, 20%, 30%, 40%, 50%, 60%, 70%, 80%, 90%, 100%, 110%, 120%, 130%, 140%, 150%, 160%, 170%, 180%, 190% more than, or 2, 3, 4, 5, 6, 7, 8, 9, 10, 11, 12, 13, 14, 15, 16, 17, 18, 19, 20, 30, 40, 50 or more fold of, that of a comparable oligonucleotide under one or more suitable conditions. In some embodiments, skipping efficiency is measured by remaining target transcript.

In some embodiments, skipping of a deleterious exon in an USH2A gene transcript is increased by at least about 10%, 15%, 20%, 25%, 30%, 35%, 40%, 45%, 50%, 60%, 70%, 75%, 80%, 85%, 90%, or 95% by administration of a provided USH2A oligonucleotide or composition thereof, e.g., at certain oligonucleotide concentrations (e.g., 1 nM, 5 nM, 10 nM, 100 nM, 500 nM, 1 uM, 5 uM, etc.) in, e.g., in vitro cell-based assays. In some embodiments, skipping of a deleterious exon in an USH2A gene transcript is increased by at least about 10%, 15%, 20%, 25%, 30%, 35%, 40%, 45%, 50%, 60%, 70%, 75%, 80%, 85%, 90%, or 95% by administration of an USH2A oligonucleotide or a composition thereof. In some embodiments, skipping of a deleterious exon in an USH2A gene transcript is increased by at least about 10%, 15%, 20%, 25%, 30%, 35%, 40%, 45%, 50%, 60%, 70%, 75%, 80%, 85%, 90%, or 95% by an USH2A oligonucleotide or a composition thereof. In some embodiments, skipping of a deleterious exon in an USH2A gene transcript is increased by at least about 10%, 15%, 20%, 25%, 30%, 35%, 40%, 45%, 50%, 60%, 70%, 75%, 80%, 85%, 90%, or 95% by administration of an USH2A oligonucleotide in vitro. In some embodiments, skipping of a deleterious exon in an USH2A gene transcript is increased by at least about 10%, 15%, 20%, 25%, 30%, 35%, 40%, 45%, 50%, 60%, 70%, 75%, 80%, 85%, 90%, or 95% by an USH2A oligonucleotide or a composition thereof in vitro. In some embodiments, skipping of a deleterious exon in an USH2A gene transcript is increased by at least about 10%, 15%, 20%, 25%, 30%, 35%, 40%, 45%, 50%, 60%, 70%, 75%, 80%, 85%, 90%, or 95% by an USH2A oligonucleotide or a composition thereof in vitro. In some embodiments, skipping of a deleterious exon in an USH2A gene transcript is increased by at least about 10%, 15%, 20%, 25%, 30%, 35%, 40%, 45%, 50%, 60%, 70%, 75%, 80%, 85%, 90%, or 95% by an USH2A oligonucleotide or a composition thereof in a cell(s) in vitro. In some embodiments, skipping of a deleterious exon in an USH2A gene transcript is increased by at least about 10%, 15%, 20%, 25%, 30%, 35%, 40%, 45%, 50%, 60%, 70%, 75%, 80%, 85%, 90%, or 95% by administration of an USH2A oligonucleotide or a composition thereof at a concentration (e.g., an oligonucleotide concentration) of 100 uM or less in a cell(s) in vitro. In some embodiments, skipping of a deleterious exon in an USH2A gene transcript is increased by at least about 10%, 15%, 20%, 25%, 30%, 35%, 40%, 45%, 50%, 60%, 70%, 75%, 80%, 85%, 90%, or 95% by an USH2A oligonucleotide or a composition thereof at an USH2A oligonucleotide concentration of 50 uM or less in a cell(s) in vitro. In some embodiments, skipping of a deleterious exon in an USH2A gene transcript is increased by at least about 10%, 15%, 20%, 25%, 30%, 35%, 40%, 45%, 50%, 60%, 70%, 75%, 80%, 85%, 90%, or 95% by an USH2A oligonucleotide or a composition thereof at a concentration of 10 uM or less in a cell(s) in vitro. In some embodiments, skipping of a deleterious exon in an USH2A gene transcript is increased by at least about 10%, 15%, 20%, 25%, 30%, 35%, 40%, 45%, 50%, 60%, 70%, 75%, 80%, 85%, 90%, or 95% by administration of an USH2A oligonucleotide or a composition thereof at a concentration of 5 uM or less in a cell(s) in vitro. In some embodiments, skipping of a deleterious exon in an USH2A gene transcript is increased by at least about 10%, 15%, 20%, 25%, 30%, 35%, 40%, 45%, 50%, 60%, 70%, 75%, 80%, 85%, 90%, or 95% by an USH2A oligonucleotide or a composition thereof at a concentration of 1 uM or less in a cell(s) in vitro. In some embodiments, an USH2A oligonucleotide or a composition thereof is capable of mediating an increase in the level of skipping of a deleterious exon in an USH2A gene transcript at a concentration of 500 nm or less in a cell in vitro. In some embodiments, an USH2A oligonucleotide or a composition thereof is capable of mediating an increase in the level of skipping of a deleterious exon in an USH2A gene transcript at a concentration of 100 nm or less in a cell in vitro. In some embodiments, an USH2A oligonucleotide or a composition thereof is capable of mediating an increase in the level of skipping of a deleterious exon in an USH2A gene transcript at a concentration of 50 nm or less in a cell in vitro.

In some embodiments, the pattern of stereochemistry of a provided USH2A oligonucleotide comprises a pattern of stereochemistry described herein or any portion thereof. In some embodiments, an oligonucleotide comprises a pattern of stereochemistry described herein and is capable of directing skipping of a deleterious exon in an USH2A gene transcript. In some embodiments, a provided USH2A oligonucleotide comprises a pattern of stereochemistry described herein and is capable of directing skipping of a deleterious exon in an USH2A gene transcript.

In some embodiments, a provided USH2A oligonucleotide comprises a modification or pattern of modification described herein. In some embodiments, a provided USH2A oligonucleotide comprises a pattern of modification described herein and is capable of directing skipping of a deleterious exon in an USH2A gene transcript. In some embodiments, a modification or pattern of modification is a modification or pattern of modification of sugar modifications, e.g., modifications at the 2′ position of sugars (e.g., 2′-F, 2′-OMe, 2′-MOE, etc.).

The ability of various USH2A to mediate skipping of exon 13 in vitro is shown in various Tables in the Example section as examples.

In some experiments, a comparator USH2A oligonucleotide is used: WV-20781, which has a linkage backbone of only PS, is stereorandom in the linkage backbone, and each sugar modification is 2′-MOE.

In various experiments, various novel USH2A oligonucleotides were constructed and tested which have different components absent from WV-20781, including: a different linkage backbone (including non-negatively charged internucleotidic linkages and natural phosphate linkages), stereochemistry in the linkage backbone (e.g., chirally controlled internucleotidic linkages in the Sp or Rp configuration), and different sugar modifications (e.g., 2′-F or 2′-OMe), and/or a different base sequence and/or length. In some experiments, a novel USH2A oligonucleotide has a higher skipping efficiency than WV-20781.

As shown in the various Tables, various USH2A oligonucleotides were capable of mediating skipping of exon 13 in an USH2A transcript. Non-limiting examples of such oligonucleotides include but are not limited to: WV-20891, WV-20892, WV-20902, WV-20908, WV-20988, WV-21008, WV-24297, WV-24368, WV-24376, WV-24366, WV-24375, WV-24360, WV-24298, WV-24381, WV-24382, WV-21100, WV-21105, and WV-20885. In at least some cases and in at least some experiments, various USH2A oligonucleotides or oligonucleotide compositions described herein had a higher skipping efficiency that comparator WV-20781.

In addition, and without wishing to be bound by any particular theory, the present disclosure notes that a low level of exon skipping occurs endogenously. Reportedly, in human cells, a low level of skipping of exon 13 of USH2A gene transcripts endogenously occurs, in addition to a low level of exon 12 skipping. Skipping of exon 13 is productive in that it produces a transcript from which an internally truncated but at least partially functional USH2A protein can be translated. Skipping of exon 12 is not productive; reportedly, skipping of exon 12 does not produce a transcript from which an at least partially functional USH2A protein can be translated.

Without wishing to be bound by any theory, the present disclosure notes that an USH2A oligonucleotide that skips both exon 12 and exon 13 would not produce a transcript from which an at least partially functional USH2A protein can be translated.

As shown in various Tables in the Example section, various USH2A oligonucleotides which are capable of mediating skipping of USH2A exon 13 were also tested for their level of skipping of exon 12. The results are shown in the Table below:

| | Fold change | | Fold change |
|---|---|---|---|
| | (skipping of | | (skipping of |
| | exon 13/exon | | exon 13/exon |
| Oligonucleotide | 12 and 13) | Oligonucleotide | 12 and 13) |
| WV-AE962 | 0.0 | WV-20891 | 2.7 |
| WV-21100 | 2.5 | WV-20892 | 3.2 |
| WV-21105 | 2.3 | WV-20902 | 4.1 |
| WV-20781 | 2.1 | WV-20908 | 4.4 |
| WV-20917 | 0.2 | WV-20988 | 2.6 |
| WV-20885 | 3.4 | WV-21008 | 0.1 |

WV-AE962 is a negative control which does not target USH2A.

Comparator oligonucleotide WV-20781 showed a ratio of skipping exon 13 compared to skipping exon 12+exon 13 of 2.1. Several of the novel USH2A oligonucleotides showed an even specificity (e.g., higher ratio of skipping exon 13 compared to skipping exon 12+exon 13). Non-limiting examples of such oligonucleotides include but are not limited to: WV-2110, WV-21105, WV-20885, WV-20891, WV-20892, WV-20902, WV-20908, and WV-20988.

### Characterization and Assessment

In some embodiments, properties and/or activities of provided oligonucleotides, e.g., USH2A oligonucleotides, and compositions thereof can be characterized and/or assessed using various technologies available to those skilled in the art, e.g., biochemical assays (e.g., exon skipping assays), cell based assays, animal models, clinical trials, etc.

In some embodiments, a method of identifying and/or characterizing an oligonucleotide composition, e.g., an USH2A oligonucleotide composition, comprises steps of:
providing at least one composition comprising a plurality of oligonucleotides; and
assessing delivery relative to a reference composition.

In some embodiments, the present disclosure provides a method of identifying and/or characterizing an oligonucleotide composition, e.g., an USH2A oligonucleotide composition, comprises steps of:
providing at least one composition comprising a plurality of oligonucleotides; and
assessing cellular uptake relative to a reference composition.

In some embodiments, the present disclosure provides a method of identifying and/or characterizing an oligonucleotide composition, e.g., an USH2A oligonucleotide composition, comprises steps of:
providing at least one composition comprising a plurality of oligonucleotides; and
assessing an increase in the level of skipping of a deleterious exon in an USH2A gene transcript.

In some embodiments, properties and/or activities of oligonucleotides, e.g., USH2A oligonucleotides, and compositions thereof are compared to reference oligonucleotides and compositions thereof, respectively.

In some embodiments, a reference oligonucleotide composition is a stereorandom oligonucleotide composition. In some embodiments, a reference oligonucleotide composition is a stereorandom composition of oligonucleotides of which all internucleotidic linkages are phosphorothioate. In some embodiments, a reference oligonucleotide composition is a DNA oligonucleotide composition with all phosphate linkages. In some embodiments, a reference oligonucleotide composition is otherwise identical to a provided chirally controlled oligonucleotide composition except that it is not chirally controlled. In some embodiments, a reference oligonucleotide composition is otherwise identical to a provided chirally controlled oligonucleotide composition except that it has a different pattern of stereochemistry. In some embodiments, a reference oligonucleotide composition is similar to a provided oligonucleotide composition except that it has a different modification of one or more sugar, base, and/or internucleotidic linkage, or pattern of modifications. In some embodiments, an oligonucleotide composition is stereorandom and a reference oligonucleotide composition is also stereorandom, but they differ in regards to sugar and/or base modification(s) or patterns thereof.

In some embodiments, a reference composition is a composition of oligonucleotides having the same base sequence and the same chemical modifications. In some embodiments, a reference composition is a composition of oligonucleotides having the same base sequence and the same pattern of chemical modifications. In some embodiments, a reference composition is a non-chirally controlled (or stereorandom) composition of oligonucleotides having the same base sequence and chemical modifications. In some embodiments, a reference composition is a non-chirally controlled (or stereorandom) composition of oligonucleotides of the same constitution but is otherwise identical to a provided chirally controlled oligonucleotide composition.

In some embodiments, the suffix “r” is appended to the designation of a stereorandom oligonucleotide composition. In some embodiments, the suffix “p” is appended to the designation of a chirally-controlled (or stereopure) oligonucleotide composition. The suffixes “r” and “p” are optional.

In some embodiments, a reference composition is a composition of oligonucleotides having the same base sequence but different chemical modifications, including but not limited to chemical modifications described herein. In some embodiments, a reference composition is a composition of oligonucleotides having the same base sequence but different patterns of internucleotidic linkages and/or stereochemistry of internucleotidic linkages and/or chemical modifications.

Various methods are known in the art for detection of gene products, the expression, level and/or activity of which may be altered after introduction or administration of a provided oligonucleotide. For example, transcripts and their variants in which an exon is skipped can be detected and quantified with qPCR, and protein levels can be determined via Western blot.

In some embodiments, assessment of efficacy of oligonucleotides can be performed in biochemical assays or in vitro in cells. In some embodiments, provided oligonucleotides can be introduced to cells via various methods available to those skilled in the art, e.g., gymnotic delivery, transfection, lipofection, etc.

In some embodiments, the efficacy of a putative USH2A oligonucleotide can be tested in vitro.

In some embodiments, the efficacy of a putative USH2A oligonucleotide can be tested in vitro using any known method of testing the expression, level and/or activity of an USH2A gene transcript or gene product thereof.

In some embodiments, the efficacy of an oligonucleotide, e.g., an USH2A oligonucleotide, can be tested in retinas (e.g., from non-human primates, or from humans) ex vivo.

In some embodiments, an USH2A oligonucleotide is tested in a cell or animal model of Usher Syndrome.

In some embodiments, a cell is a patient-derived fibroblast cell. Fibroblasts from an USH2 patient, having the USH2A c.7595-2144A>G (p.Lys2532Thrfs*56) and c.10636G>A (p.Gly3546Arg) mutations in compound heterozygosity, have been reported, and can be used to evaluate USH2A oligonucleotides.

In some embodiments, an animal model of Usher Syndrome or RP is a cynomolgus monkey.

The capable of USH2A oligonucleotides to skip exon 13 can be tested in the retina of cynomolgus monkeys mediated exon 12 (which equivalent to human exon 13). As a non-limiting example: Wild-type cynomolgus monkeys can receive one or more IVT injections (bilateral) of a dose of an USH2A oligonucleotide. Retina samples can be collected at multiple time points post-injection (e.g., 1 hour, 12 hours, 15 days, 28 days and 102 days) for assessment of exon skipping. Retinas can separated from the eyes, RNA can be isolated, and the levels of USH2A transcripts with and without exon 12 can be quantified using isoform specific ddPCR assays and the percentage of exon skipping can be calculated.

In some embodiments, an animal model of Usher Syndrome or RP is a zebrafish.

As a non-limiting example: A zebrafish model, homozygous for exon 13 premature stop codon mutation (referred as to USH2Armc1), can be used to assess the activity of the usherin protein resulting from the exon 13 skiping in USH2A mRNA. USH2Armc1 zebrafish larvae reportedly have no functional usherin protein and show a significantly reduced b-wave amplitude in electroretinogram (ERG) recordings. USH2Armc1 zebrafish can be treated with zebrafish-specific oligonucleotides followed by assessment of exon skipping, usherin protein localization, and recording of the ERG b-wave amplitude.

In some embodiments, an animal model of Usher Syndrome or RP is a mouse.

As a non-limiting example: Wild-type mice can receive bilateral IVT injection of an USH2A oligonucleotide to assess in vivo delivery, eyes can be fixed overnight in Hartmann's fixative and embedded in paraffin. USH2A oligonucleotides can be visualized in retina using complementary probe with Cy5 label by in situ hybridization. Images can be acquired on a LSM800 confocal microscope.

In some embodiments, a cell is Usher Syndrome patient-derived cell.

Many technologies for assessing activities and/or properties of oligonucleotides in animals are known and practiced by those skilled in the art and can be utilized in accordance with the present disclosure. In some embodiments, evaluation of an oligonucleotide can be performed in an animal. Various animals may be used to assess properties and activities of provided oligonucleotides and compositions thereof.

Identification of the USH2A gene has allowed for the development of animal models of the disease, including a transgenic animal model carrying mutated human or mouse forms of the gene. Models include mice carrying at least a portion of the human gene, which contains the disease-associated mutations (or the wild-type equivalent). Animal models typically have at least some shared features with the human disease. These mice have allowed for the testing of a number of different therapeutic agents for the prevention, amelioration and treatment of Usher Syndrome using a number of endpoints. Useful compounds may function by a number of different mechanisms.

Various animal models of Usher Syndrome have been reported in the literature. For information related to cells, cell lines, animal models, including but not limited to mice, rats and flies, and various experimental procedures suitable for the study of USH2A, and/or the analysis of USH2A oligonucleotides, see those noted herein or in the relevant art. Various model organisms have reportedly been used in the study of USH2A function. Any of these model organisms can be used to analyze the activity or other properties of an USH2A oligonucleotide.

In some embodiments, an animal model administered an USH2A oligonucleotide can be evaluated for safety and/or efficacy.

In some embodiments, the effect(s) of administration of an oligonucleotide to an animal can be evaluated, including any effects on behavior, inflammation, and toxicity. In some embodiments, following dosing, animals can be observed for signs of toxicity including trouble grooming, lack of food consumption, and any other signs of lethargy. In some embodiments, in a mouse model of Usher Syndrome (e.g., Usher Syndrome Type 2A), following administration of an USH2A oligonucleotide, the animals can be monitored for timing of onset of a rear paw clasping phenotype.

In some embodiments, following administration of an USH2A oligonucleotide to an animal, the animal can be sacrificed and analysis of tissues or cells can be performed to determine changes in mutant or wild-type USH2A, or other biochemical or other changes. In some embodiments, following necropsy, liver, heart, lung, kidney, and spleen can be collected, fixed, and processed for histopathological evaluation (standard light microscopic examination of hematoxylin and eosin-stained tissue slides).

In some embodiments, following administration of an USH2A oligonucleotide to an animal, behavioral changes can be monitored or assessed. In some embodiments, such an assessment can be performed using a technique described in the scientific literature.

Various effects of testing in animals described herein can also be monitored in human subjects or patients following administration of an USH2A oligonucleotide.

In addition, the efficacy of an USH2A oligonucleotide in a human patient can be measured by evaluating, after administration of the oligonucleotide, any of various parameters known in the art, including but not limited to a reduction in a symptom of Usher Syndrome, or a decrease in the rate of worsening of a symptom of Usher Syndrome.

In some embodiments, following human treatment with an oligonucleotide, or contacting a cell or tissue in vitro with an oligonucleotide, cells and/or tissues are collected for analysis.

In some embodiments, in various cells and/or tissues, target nucleic acid levels can be quantitated by methods available in the art, many of which can be accomplished with commercially available kits and materials. Such methods include, e.g., Northern blot analysis, competitive polymerase chain reaction (PCR), quantitative real-time PCR, etc. RNA analysis can be performed on total cellular RNA or poly(A)+ mRNA. Probes and primers are designed to hybridize to a nucleic acid to be detected. Methods for designing real-time PCR probes and primers are well known and widely practiced in the art. For example, to detect and quantify USH2A RNA, an example method comprises isolation of total RNA (e.g., including mRNA) from a cell or animal treated with an oligonucleotide or a composition and subjecting the RNA to reverse transcription and/or quantitative real-time PCR, for example, as described herein, or in: Moon et al. 2012 Cell Metab. 15: 240-246.

In some embodiments, protein levels can be evaluated or quantitated in various methods known in the art, e.g., enzyme-linked immunosorbent assay (ELISA), Western blot analysis (immunoblotting), immunocytochemistry, fluorescence-activated cell sorting (FACS), immunohistochemistry, immunoprecipitation, protein activity assays (for example, caspase activity assays), and quantitative protein assays. Antibodies useful for the detection of mouse, rat, monkey, and human proteins are commercially available or can be generated if needed. For example, various USH2A antibodies have been reported in the literature. Antibodies to USH2A are also commercially available, e.g., from LifeSpan BioSciences (Seattle, WA), Abcam (Cambridge, MA), Santa Cruz BioTechnology (Santa Cruz, CA), etc.

Various technologies are available and/or known in the art for detecting levels of oligonucleotides or other nucleic acids. Such technologies are useful for detecting provided oligonucleotides, e.g., USH2A oligonucleotides, when administered to assess, e.g., delivery, cell uptake, stability, distribution, etc.

In some embodiments, selection criteria are used to evaluate the data resulting from various assays and to select particularly desirable oligonucleotides, e.g., desirable USH2A oligonucleotides, with certain properties and activities. In some embodiments, selection criteria for a stability assay include at least 50% stability [at least 50% of an oligonucleotide is still remaining and/or detectable] at Day 1. In some embodiments, selection criteria for a stability assay include at least 50% stability at Day 2. In some embodiments, selection criteria for a stability assay include at least 50% stability at Day 3. In some embodiments, selection criteria for a stability assay include at least 50% stability at Day 4. In some embodiments, selection criteria for a stability assay include at least 50% stability at Day 5. In some embodiments, selection criteria for a stability assay include at least 80% [at least 80% of the oligonucleotide remains] at Day 5.

In some embodiments, a target gene, e.g., USH2A target gene, comprises one or more mutations.

In some embodiments, efficacy of an USH2A oligonucleotide is assessed directly or indirectly by monitoring, measuring or detecting a change in a condition, disorder or disease or a biological pathway associated with USH2A.

In some embodiments, efficacy of an USH2A oligonucleotide is assessed directly or indirectly by monitoring, measuring or detecting a change in a response to be affected by USH2A.

In some embodiments, a provided oligonucleotide (e.g., an USH2A oligonucleotide) can by analyzed by a sequence analysis to determine what other genes [e.g., genes which are not a target gene (e.g., USH2A)] have a sequence which is complementary to the base sequence of the provided oligonucleotide (e.g., the USH2A oligonucleotide) or which have 0, 1, 2 or more mismatches from the base sequence of the provided oligonucleotide (e.g., the USH2A oligonucleotide). Knockdown or exon skipping, if any, by the oligonucleotide of these potential off-targets can be determined to evaluate potential off-target effects of an oligonucleotide (e.g., an USH2A oligonucleotide). In some embodiments, an off-target effect is also termed an unintended effect and/or related to hybridization to a bystander (non-target) sequence or gene.

Oligonucleotides which have been evaluated and tested for efficacy in mediating exon skipping in USH2A have various uses, e.g., in treatment or prevention of an USH2A-related condition, disorder or disease or a symptom thereof.

In some embodiments, an USH2A oligonucleotide which has been evaluated and tested for its ability to provide a particular biological effect (e.g., reduction of level, expression and/or activity of an USH2A gene transcript comprising a deleterious exon or a gene product thereof) can be used to treat, ameliorate and/or prevent an USH2A-related condition, disorder or disease.

### Treatment of USH2A-Related Conditions, Disorders or Diseases

In some embodiments, the present disclosure provides an USH2A oligonucleotide which targets USH2A (e.g., an USH2A oligonucleotide comprising an USH2A target sequence or a sequence complementary to an USH2A target sequence). In some embodiments, the present disclosure provides an USH2A oligonucleotide which directs skipping of a deleterious exon in an USH2A gene transcript. In some embodiments, the present disclosure provides methods for preventing and/or treating USH2A-related conditions, disorders or diseases using provided USH2A oligonucleotides and compositions thereof. In some embodiments, the present disclosure provides oligonucleotides and compositions thereof for use as medicaments, e.g., for USH2A-related conditions, disorders or diseases. In some embodiments, the present disclosure provides oligonucleotides and compositions thereof for use in the treatment of USH2A-related conditions, disorders or diseases. In some embodiments, the present disclosure provides oligonucleotides and compositions thereof for the manufacture of medicaments for the treatment of USH2A-related conditions, disorders or diseases.

In some embodiments, the present disclosure provides a method for preventing, treating or ameliorating an USH2A-related condition, disorder or disease in a subject susceptible thereto or suffering therefrom, comprising administering to the subject a therapeutically effective amount of an USH2A oligonucleotide or a pharmaceutical composition thereof.

In some embodiments, a patient may be identified by a genetic screen or test for a mutation in USH2A (e.g., after a determination has been made that one or both parents are a carrier or are afflicting with an USH2A-related disease, disorder or condition), but at an early stage in disease progression or before any symptoms have appeared. In some embodiments, the present disclosure pertains to a method of administering an USH2A oligonucleotide to a patient who is susceptible to (e.g., has a genetic mutation related to) an USH2A-related disease, disorder or condition, and administration of the oligonucleotide is capable of delaying onset of or prevent worsening of a symptom of the disease, disorder or condition.

In some embodiments, the present disclosure provides a method for treating or ameliorating an USH2A-related condition, disorder or disease in a subject suffering therefrom, comprising administering to the subject a therapeutically effective amount of an USH2A oligonucleotide or a pharmaceutical composition thereof.

In some embodiments, an USH2A-related condition, disorder or disease is Usher Syndrome (e.g., Usher Syndrome Type 2A), atypical Usher syndrome, or nonsyndromic (non-syndromic) retinitis pigmentosa (e.g., nonsyndromic autosomal recessive retinitis pigmentosa (AARP).

In some embodiments, the present disclosure provides a method for reducing USH2A gene expression in a cell, comprising: contacting the cell with an USH2A oligonucleotide or a composition thereof. In some embodiments, the present disclosure provides a method for reducing the level of an USH2A gene transcript in a cell, comprising: contacting the cell with an USH2A oligonucleotide or a composition thereof. In some embodiments, the present disclosure provides a method for reducing the level of an USH2A protein in a cell, comprising: contacting the cell with an USH2A oligonucleotide or a composition thereof. In some embodiments, provided methods selectively reduce levels of USH2A transcripts and/or products encoded thereby that are related to conditions, disorders or diseases.

In some embodiments, the present disclosure provides a method for increasing the level of skipping of a deleterious USHA exon in a mammal in need thereof, comprising administering to the mammal a nucleic acid-lipid particle comprising a provided USH2A oligonucleotide or a composition thereof.

In some embodiments, the present disclosure provides a method for in vivo delivery of an USH2A oligonucleotide, comprising administering to a mammal an USH2A oligonucleotide or a composition thereof.

In some embodiments, a mammal is a human. In some embodiments, a mammal is susceptible to or afflicted with and/or suffering from an USH2A-related condition, disorder or disease. In some embodiments, a mammal susceptible to an USH2A-related condition, disorder or disease has a familial history of such a condition, disorder or disease, and/or has been genetically tested and determined to comprise a CAG expansion in the USH2A gene.

In some embodiments, a subject or patient suitable for treatment of an USH2A-related condition, disorder or disease, such as Usher Syndrome (e.g., Usher Syndrome Type 2A), atypical Usher syndrome, or nonsyndromic retinitis pigmentosa, can be identified or diagnosed by a health care professional.

In some embodiments, a symptom of Usher Syndrome (e.g., Usher Syndrome Type 2A), atypical Usher syndrome, or nonsyndromic retinitis pigmentosa is any symptom listed herein.

In some embodiments, a provided oligonucleotide or a composition thereof prevents, treats, ameliorates, or slows progression of an USH2A-related condition, disorder or disease, or at least one symptom of an USH2A-related condition, disorder or disease.

In some embodiments, a method of the present disclosure is for the treatment of Usher Syndrome (e.g., Usher Syndrome Type 2A), atypical Usher syndrome, or nonsyndromic retinitis pigmentosa in a subject wherein the method comprises administering to a subject a therapeutically effective amount of an USH2A oligonucleotide or a pharmaceutical composition thereof.

In some embodiments, a provided method reduces at least one symptom of Usher Syndrome (e.g., Usher Syndrome Type 2A), atypical Usher syndrome, or nonsyndromic retinitis pigmentosa wherein the method comprises administering to a subject a therapeutically effective amount of an USH2A oligonucleotide or a pharmaceutical composition thereof.

In some embodiments, the present disclosure provides a method for treating and/or ameliorating one or more symptoms associated with an USH2A-related condition, disorder or disease in a mammal in need thereof, the method comprising administering to the mammal a therapeutically effective amount of an USH2A oligonucleotide or a composition thereof. In some embodiments, the present disclosure provides a method for reducing susceptibility to an USH2A-related condition, disorder or disease in a mammal in need thereof, the method comprising: administering to the mammal a therapeutically effective amount of an USH2A oligonucleotide or a composition thereof. In some embodiments, the present disclosure provides a method for preventing or delaying the onset of an USH2A-related condition, disorder or disease in a mammal in need thereof, the method comprising: administering to the mammal a therapeutically effective amount of an USH2A oligonucleotide or a composition thereof. In some embodiments, the present disclosure provides a method for treating and/or ameliorating one or more symptoms associated with an USH2A-related condition, disorder or disease in a mammal in need thereof, the method comprising: administering to the mammal a therapeutically effective amount of a nucleic acid-lipid particle comprising an USH2A oligonucleotide. In some embodiments, the present disclosure provides a method for reducing susceptibility to an USH2A-related condition, disorder or disease in a mammal in need thereof, the method comprising: administering to the mammal a therapeutically effective amount of a nucleic acid-lipid particle comprising an USH2A oligonucleotide. In some embodiments, the present disclosure provides a method for preventing or delaying the onset of an USH2A-related condition, disorder or disease in a mammal in need thereof, the method comprising: administering to the mammal a therapeutically effective amount of a nucleic acid-lipid particle comprising an USH2A oligonucleotide. In some embodiments, a mammal is a human. In some embodiments, a mammal is susceptible to, afflicted with and/or suffering from an USH2A-related condition, disorder or disease.

In some embodiments, administration of an USH2A oligonucleotide to a patient or subject is capable of mediating any one or more of: slowing Usher Syndrome (e.g., Usher Syndrome Type 2A), atypical Usher syndrome, or nonsyndromic retinitis pigmentosa progression, delaying the onset of Usher Syndrome or at least one symptom thereof, improving one or more indicators of Usher Syndrome, and/or increasing the survival time or lifespan of the patient or subject.

In some embodiments, slowing disease progression relates to the prevention of, or delay in, a clinically undesirable change in one or more clinical parameters in an individual susceptible to or suffering from Usher Syndrome, such as those described herein. It is well within the abilities of a physician to identify a slowing of disease progression in an individual susceptible to or suffering from Usher Syndrome, using one or more of the disease assessment tests described herein. Additionally, it is understood that a physician may administer to the individual diagnostic tests other than those described herein to assess the rate of disease progression in an individual susceptible to or suffering from Usher Syndrome.

In some embodiments, delaying the onset of Usher Syndrome or a symptom thereof relates to delaying one or more undesirable changes in one or more indicators of Usher Syndrome that are negative for Usher Syndrome. A physician may use family history of Usher Syndrome or comparisons to other Usher Syndrome patients with similar genetic profile to determine an expected approximate age of Usher Syndrome onset to Usher Syndrome to determine if onset of Usher Syndrome is delayed.

In some embodiments, indicators of Usher Syndrome include parameters employed by a medical professional, such as a physician, to diagnose or measure the progression of Usher Syndrome.

In some embodiments, an improvement in an indicator of Usher Syndrome relates to the absence of an undesirable change, or the presence of a desirable change, in one or more indicators of Usher Syndrome. In one embodiment, an improvement in an indicator of Usher Syndrome is evidenced by the absence of a measurable change in one or more indicators of Usher Syndrome. In another embodiment, an improvement in an indicator of Usher Syndrome is evidenced by a desirable change in one or more indicators of Usher Syndrome.

In some embodiments, a slowing of disease progression may further comprise an increase in survival time in an individual susceptible to or suffering from Usher Syndrome. In some embodiments, an increase in survival time relates to mean increasing the survival of an individual suffering from Usher Syndrome, relative to an approximate survival time based upon Usher Syndrome progression and/or family history of Usher Syndrome. A physician can use one or more of the disease assessment tests described herein to predict an approximate survival time of an individual susceptible to or suffering from Usher Syndrome. A physician may additionally use the family history of an individual susceptible to or suffering from Usher Syndrome or comparisons to other Usher Syndrome patients with similar genetic profile to predict expected survival time.

In some embodiments, the present disclosure provides a method of inhibiting USH2A expression in a cell, the method comprising: (a) contacting the cell with an USH2A oligonucleotide; and (b) maintaining the cell produced in step (a) for a time sufficient to obtain degradation of a mRNA transcript of an USH2A gene, thereby inhibiting expression of the USH2A gene in the cell. In some embodiments, USH2A expression is inhibited by at least 30%.

In some embodiments, the present disclosure provides a method of treating a condition, disorder or disease mediated by USH2A expression comprising administering to a human susceptible to or suffering therefrom a therapeutically effective amount of an USH2A oligonucleotide or a composition thereof. In some embodiments, administration causes an increase the level of skipping of a deleterious exon in an USH2A transcript. In some embodiments, administration is associated with an increase the level of skipping of a deleterious exon in an USH2A transcript. In some embodiments, administration is followed by an increase in the level of skipping of a deleterious exon in an USH2A gene transcript.

In some embodiments, the present disclosure provides an USH2A oligonucleotide for use in a subject to treat an USH2A-related condition, disorder or disease. In some embodiments, an USH2A-related condition, disorder or disease is selected from Usher Syndrome (e.g., Usher Syndrome Type 2A), atypical Usher syndrome, or nonsyndromic retinitis pigmentosa.

In some embodiments, a subject is administered an USH2A oligonucleotide or a composition thereof and an additional agent and/or method, e.g., an additional therapeutic agent and/or method. In some embodiments, an oligonucleotide or composition thereof can be administered alone or in combination with one or more additional therapeutic agents and/or treatment. When administered in combination each component may be administered at the same time or sequentially in any order at different points in time. In some embodiments, each component may be administered separately but sufficiently closely in time so as to provide the desired therapeutic effect. In some embodiments, provided oligonucleotides and additional therapeutic components are administered concurrently. In some embodiments, provided oligonucleotides and additional therapeutic components are administered as one composition. In some embodiments, at a time point a subject being administered is exposed to both provided oligonucleotides and additional components at the same time.

In some embodiments, an oligonucleotide and/or an additional therapeutic agent is delivered by intravitreal injection. In some embodiments, prior to intravitreal injection, a mydriatic (e.g., 1% tropicamide) is instilled in the eye, followed by a topical anesthetic.

In some embodiments, an additional therapeutic agent or method is capable of preventing, treating, ameliorating or slowing the progress of a neurological condition, disorder or disease. In some embodiments, an additional therapeutic agent or method is capable of preventing, treating, ameliorating or slowing the progress of an USH2A-related condition, disorder or disease. In some embodiments, an additional therapeutic agent or method may “indirectly” mediate an increase in the level of skipping of a deleterious exon in USH2A.

In some embodiments, an additional therapeutic agent is physically conjugated to an oligonucleotide, e.g., an USH2A oligonucleotide. In some embodiments, an additional agent is an USH2A oligonucleotide. In some embodiments, a provided oligonucleotide is physically conjugated with an additional agent which is an USH2A oligonucleotide. In some embodiments, additional agent oligonucleotides have base sequences, sugars, nucleobases, internucleotidic linkages, patterns of sugar, nucleobase, and/or internucleotidic linkage modifications, patterns of backbone chiral centers, etc., or any combinations thereof, as described in the present disclosure, wherein each U may be independently replaced with T and vice versa. In some embodiments, an additional oligonucleotide targets USH2A. In some embodiments, an USH2A oligonucleotide is physically conjugated to a second oligonucleotide which can mediate an increase in the level of skipping of a deleterious exon in USH2A, or which is useful for treating an USH2A-related condition, disorder or disease. In some embodiments, a first USH2A oligonucleotide is physically conjugated to a second USH2A oligonucleotide, which can be identical to the first USH2A oligonucleotide or not identical, and which can target a different or the same or an overlapping sequence as the first USH2A oligonucleotide.

In some embodiments, a provided oligonucleotide, e.g., an USH2A oligonucleotide, may be administered with one or more additional (or second) therapeutic agent for Usher Syndrome.

In some embodiments, an additional therapeutic agent comprises a treatment for one or more symptoms of Usher Syndrome.

In some embodiments, an additional treatment is a treatment intended to reduce or eliminate a symptom of Usher Syndrome, including but not limited to a symptom listed herein.

In some embodiments, an additional treatment or therapeutic is a hearing aid or cochlear implant.

In some embodiments, an additional therapeutic agent and/or method is any described or referenced in: Nguyen et al. 2014 Retinal Degenerative Diseases pp 471-476, or WO/2019/075320, WO/2019/009265, WO/2018/232227, WO/2018/213278, WO/2018/208703, WO/2018/201146, WO/2018/182527, WO/2018/172961, WO/2018/169090, WO/2018/167510, WO/2018/107226, WO/2018/100054, WO/2018/096196, WO/2018/085644, WO/2018/030389, WO/2018/009562, WO/2018/002873, WO/2017/201425, WO/2017/151823, WO/2017/144611, WO/2017/123710, WO/2017/121766, WO/2017/106364, WO/2017/048731, WO/2017/044649, WO/2017/042584, WO/2016/191645, WO/2016/145345, WO/2016/144892, WO/2016/138353, WO/2016/130460, WO/2016/077467, WO/2016/077422, WO/2016/073931, WO/2016/073829, WO/2016/017980, WO/2016/017831, WO/2016/014353, WO/2016/001693, WO/2015/160893, WO/2015/143418, WO/2015/134812, WO/2015/126972, WO/2015/110556, WO/2015/105064, WO/2015/042281, WO/2015/020522, WO/2015/001379, WO/2014/180996, WO/2014/130869, WO/2014/129466, WO/2014/100361, WO/2014/066836, WO/2014/066835, WO/2014/058464, WO/2014/011210, WO/2013/134867, WO/2013/112448, WO/2013/053719, WO/2012/167109, WO/2012/148994, WO/2012/148930, WO/2012/145708, WO/2012/135498, WO/2012/100142, WO/2012/043891, WO/2012/024404, WO/2011/149012, WO/2011/149010, WO/2011/133964, WO/2011/095475, WO/2011/025734, WO/2010/150564, WO/2010/130418, WO/2010/099436, WO/2010/097201, WO/2010/032073, WO/2010/005533, WO/2009/111169, WO/2009/102021, WO/2009/089399, WO/2009/083188, WO/2009/083185, WO/2009/047640, WO/2009/046446, WO/2009/018333, WO/2008/135536, WO/2008/125908, WO/2008/124151, WO/2008/111497, WO/2008/013983, WO/2007/131180, WO/2007/094669, WO/2007/014327, WO/2007/011880, WO/2007/011674, WO/2006/101634, WO/2006/086452, WO/2006/077824, WO/2005/120544, WO/2005/110114, WO/2005/079815, WO/2005/074981, WO/2005/023311, WO/2004/096146, WO/2004/043480, WO/2004/030693, WO/2003/105678, WO/2003/082081, WO/2003/047525, or WO/2003/007979, WO/2003/004058.

In some embodiments, an oligonucleotide or composition thereof can be administered alone or in combination with one or more additional therapeutic agents and/or treatment. When administered in combination each component may be administered at the same time or sequentially in any order at different points in time. In some embodiments, each component may be administered separately but sufficiently closely in time so as to provide the desired therapeutic effect. In some embodiments, provided oligonucleotides and additional therapeutic components are administered concurrently. In some embodiments, provided oligonucleotides and additional therapeutic components are administered as one composition. In some embodiments, at a time point a subject being administered is exposed to both provided oligonucleotides and additional components at the same time.

In some embodiments, an additional therapeutic agent or method is capable of preventing, treating, ameliorating or slowing the progress of a neurological condition, disorder or disease. In some embodiments, an additional therapeutic agent or method is capable of preventing, treating, ameliorating or slowing the progress of an USH2A-related condition, disorder or disease. In some embodiments, an additional therapeutic agent or method may “indirectly” decrease the expression, activity and/or level of USH2A, e.g., by knocking down a gene or gene product which can increases the expression, activity and/or level of USH2A.

In some embodiments, an additional therapeutic agent is physically conjugated to an oligonucleotide, e.g., an USH2A oligonucleotide. In some embodiments, an additional agent is an USH2A oligonucleotide. In some embodiments, a provided oligonucleotide is physically conjugated with an additional agent which is an USH2A oligonucleotide. In some embodiments, additional agent oligonucleotides have base sequences, sugars, nucleobases, internucleotidic linkages, patterns of sugar, nucleobase, and/or internucleotidic linkage modifications, patterns of backbone chiral centers, etc., or any combinations thereof, as described in the present disclosure, wherein each U may be independently replaced with T and vice versa. In some embodiments, an additional oligonucleotide targets USH2A. In some embodiments, an USH2A oligonucleotide is physically conjugated to a second oligonucleotide which can decrease (directly or indirectly) the expression, activity and/or level of a mutant USH2A, or which is useful for treating an USH2A-related condition, disorder or disease. In some embodiments, a first USH2A oligonucleotide is physically conjugated to a second USH2A oligonucleotide, which can be identical to the first USH2A oligonucleotide or not identical, and which can target a different or the same or an overlapping sequence as the first USH2A oligonucleotide.

In some embodiments, a provided oligonucleotide, e.g., an USH2A oligonucleotide, may be administered with one or more additional (or second) therapeutic agent for retinopathy.

In some embodiments, an additional therapeutic agent comprises a treatment for one or more symptoms of retinopathy.

In some embodiments, an additional treatment is a treatment intended to reduce or eliminate a symptom of retinopathy, including but not limited to a symptom listed herein.

In some embodiments, a subject is administered an USH2A oligonucleotide and an additional therapeutic agent, wherein the additional therapeutic agent is an agent described herein or known in the art which is useful for treatment of an USH2A-related condition, disorder or disease.

In some embodiments, an additional therapeutic agent is, as a non-limiting example: Valproic acid.

In some embodiments, an additional therapeutic agent is, as a non-limiting example: Curcumin.

In some embodiments, an additional therapeutic agent is, as a non-limiting example: proinsulin.

In some embodiments, an additional therapeutic agent is, as a non-limiting example: a ribozyme or other small nucleic acid (e.g., an antisense oligonucleotide, single- or double-stranded siRNA or RNAi agent, etc.) targeting opsin.

In some embodiments, an additional therapeutic agent is, as a non-limiting example: QR-421a.

In some embodiments, an additional therapeutic agent is, as a non-limiting example: an rAAV delivered ribozyme targeting opsin.

In some embodiments, an additional therapeutic agent is, as a non-limiting example: Transplantation of Photoreceptor and Total Neural Retina.

In some embodiments, an additional therapeutic agent is, as a non-limiting example: Gene therapy.

In some embodiments, an additional therapeutic agent is, as a non-limiting example: Transplantation of syngeneic Schwann cells to the retina.

In some embodiments, an additional therapeutic agent is, as a non-limiting example: CRISPR/Cas9 genome surgery.

In some embodiments, USH2A gene editing using the CRISPR system was reported in Fuster-Garcia et al. 1017 Mol. Ther. Nuc. Acids 8: 529.

In some embodiments, an additional therapeutic agent is, as a non-limiting example: tauroursodeoxycholic acid.

In some embodiments, an additional therapeutic agent is, as a non-limiting example: tauroursodeoxycholic acid or a derivative thereof.

In some embodiments, an additional therapeutic agent is, as a non-limiting example: 11-cis-retinal or a derivative thereof.

In some embodiments, an additional therapeutic agent is, as a non-limiting example: 11-cis-retinal or a derivative thereof or a compound described in WO/2018/201146.

In some embodiments, an additional therapeutic agent is, as a non-limiting example: a meganuclease.

In some embodiments, an additional therapeutic agent is, as a non-limiting example: an indene derivative.

In some embodiments, an additional therapeutic agent is, as a non-limiting example: an indene derivative described in EP3176163.

In some embodiments, an additional therapeutic agent is, as a non-limiting example: a Pyrazolopyridazine or a derivative thereof.

In some embodiments, an additional therapeutic agent is, as a non-limiting example: a Pyrazolopyridazine or a derivative thereof described in U.S. Pat. No. 9,925,187.

In some embodiments, an additional therapeutic agent is, as a non-limiting example: gasdermin, gasdermin A, gasdermin B, gasdermin C, gasdermin D, DFNA5 or DFNB59 (or pejvakin), or a derivative of any of these compounds.

In some embodiments, an additional therapeutic agent is, as a non-limiting example: a methanone derivative and/or a benzo-thiophene derivative.

In some embodiments, an additional therapeutic agent is, as a non-limiting example: a methanone derivative described in WO/2016/017831.

In some embodiments, an additional therapeutic agent is, as a non-limiting example: a RDCVF1 or a RDCVF2 protein or a nucleic acid encoding the same.

In some embodiments, an additional therapeutic agent is, as a non-limiting example: a PRO polypeptide.

In some embodiments, an additional therapeutic agent is, as a non-limiting example: a beta- or gamma-diketone or an analog or derivative thereof.

In some embodiments, an additional therapeutic agent is, as a non-limiting example: a retinoic acid receptor agonistic action (such as tamibarotene, tamibarotene methyl ester, tamibarotene ethyl ester, tazarotene, tazarotenic acid, adapalene, palovalotene, retinol, isotretinoin, alitretinoin, etretinate, acitretin, and bexarotene) or a salt thereof.

In some embodiments, an additional therapeutic agent is, as a non-limiting example: a tetra- or pentapeptide.

In some embodiments, an additional therapeutic agent is, as a non-limiting example: a tetra- or pentapeptide described in US20180353565.

In some embodiments, an additional therapeutic agent is, as a non-limiting example: a compound capable of inhibiting YHL/Vhl.

In some embodiments, an additional therapeutic agent is, as a non-limiting example: N-acetylcysteine amide.

In some embodiments, an additional therapeutic agent is, as a non-limiting example: 1,2,4-oxadiazole benzoic acid compounds.

In some embodiments, an additional therapeutic agent is, as a non-limiting example: 3-[5-(2-fluorophenyl)-[1,2,4]oxadiazol-3-yl]benzoic acid.

In some embodiments, an additional therapeutic agent is, as a non-limiting example: 7,8-dihydroxyflavone (DHF).

In some embodiments, an additional therapeutic agent is, as a non-limiting example: 9- or 11-cis retinyl ester.

In some embodiments, an additional therapeutic agent is, as a non-limiting example: a benzaldehyde compound.

In some embodiments, an additional therapeutic agent is, as a non-limiting example: a compound described in U.S. Pat. App. No. US20110224200.

In some embodiments, an additional therapeutic agent is, as a non-limiting example: a compound described in WO2010150564.

In some embodiments, an additional therapeutic agent is, as a non-limiting example: a compound or a combination of lutein, zeaxanthin, glutathione, and/or alpha lipoic acid.

In some embodiments, an additional therapeutic agent is, as a non-limiting example: a compound or medicament described in Chinese Patent App. No. 201510118534.9.

In some embodiments, an additional therapeutic agent is, as a non-limiting example: a method or composition described in US20160058825.

In some embodiments, an additional therapeutic agent is, as a non-limiting example: a phthalazinone pyrazole derivative.

In some embodiments, an additional therapeutic agent is, as a non-limiting example: a proteasomal inhibitor selected from the group consisting of MG-132, lactocystin, clastolactocystin beta lactone, PSI, MG-115, MG101, N-acetyl-Leu-Leu-Met-CHO, N-carbobenzoyl-Gly-Pro-Phe-Leu-CHO, N-carbobenzoyl-Gly-Pro-Ala-Phe-CHO, or N-carbobenzoyl-Leu-Leu-Phe-CHO or a salt thereof.

In some embodiments, an additional therapeutic agent is, as a non-limiting example: a proteasomal inhibitor selected from the group consisting of MG-132, lactocystin, clastolactocystin beta lactone, PSI, MG-115, MG101, N-acetyl-Leu-Leu-Met-CHO, N-carbobenzoyl-Gly-Pro-Phe-Leu-CHO, N-carbobenzoyl-Gly-Pro-Ala-Phe-CHO, or N-carbobenzoyl-Leu-Leu-Phe-CHO or a salt thereof, in combination with a compound selected from the group consisting of 11-cis-retinal, 9-cis-retinal or a 7-ring locked isomer of 11-cis retinal.

In some embodiments, an additional therapeutic agent is, as a non-limiting example: a pyridine-3-carbaldehyde-0-(piperidin-1-yl-propyl)-oxime derivative.

In some embodiments, an additional therapeutic agent is, as a non-limiting example: a serine palmitoyltransferase inhibitor.

In some embodiments, an additional therapeutic agent is, as a non-limiting example: an alpha1 receptor blocker.

In some embodiments, an additional therapeutic agent is, as a non-limiting example: an apoptosis suppressing agent.

In some embodiments, an additional therapeutic agent is, as a non-limiting example: an apoptosis suppressing agent containing (R)-1-(benzofuran-2-yl)-2-propylaminopentane or its pharmacologically permissible salt, hydrate or solvate.

In some embodiments, an additional therapeutic agent is, as a non-limiting example: an aromatic-cationic peptide.

In some embodiments, an additional therapeutic agent is, as a non-limiting example: an aromatic-cationic peptide represented by the formula D-Arg-2′,6′-Dmt-Lys-Phe-NH2 (SS-31) or Phe-D-Arg-Phe-Lys-NH2 (SS-20).

In some embodiments, an additional therapeutic agent is, as a non-limiting example: an IL-6 inhibitor, an APOE inhibitor and/or a Fas activator.

In some embodiments, an additional therapeutic agent is, as a non-limiting example: an indazole derivative.

In some embodiments, an additional therapeutic agent is, as a non-limiting example: an Inhibitor of TGF-R-signaling.

In some embodiments, an additional therapeutic agent is, as a non-limiting example: an isoquinoline sulfonyl derivative.

In some embodiments, an additional therapeutic agent is, as a non-limiting example: an opioid antagonist.

In some embodiments, an additional therapeutic agent is, as a non-limiting example: an SIP receptor agonist.

In some embodiments, an additional therapeutic agent is, as a non-limiting example: an SIP receptor agonist such as 2-amino-2-[2-(4-octylphenyl)ethyl]propane-1,3-diol and (2R)-2-amino-4-[3-(4-cyclohexyloxybutyl)-benzo[b]thien-6-yl]-2-methylbutan-1-ol.

In some embodiments, an additional therapeutic agent is, as a non-limiting example: benzo-thiophene derivative.

In some embodiments, an additional therapeutic agent is, as a non-limiting example: dopamine and/or serotonin receptor antagonist.

In some embodiments, an additional therapeutic agent is, as a non-limiting example: fragments of the histone deacetylase 4 (HDAC4) gene lacking the enzymatic domain.

In some embodiments, an additional therapeutic agent is, as a non-limiting example: geranylgeranylacetone.

In some embodiments, an additional therapeutic agent is, as a non-limiting example: insulin, IGF-1, and/or chlorin e6.

In some embodiments, an additional therapeutic agent is, as a non-limiting example: N-acetylcysteine amide (NACA).

In some embodiments, an additional therapeutic agent is, as a non-limiting example: nut and/or seed oils, walnut oil, almond oil, avocado oil, pistachio oil and/or flaxseed oil, or any combination thereof.

In some embodiments, an additional therapeutic agent is, as a non-limiting example: pigment epithelium-derived factor (PEDF) and/or docosahexaenoic acid (DHA).

In some embodiments, an additional therapeutic agent is, as a non-limiting example: somatostatin-28, somatostatin-14, somatostatin-13, prosomatostatin, octreotide, lanreotide, vapreotide, pasireotide, seglitide, or cortistatin or any of their pharmaceutically acceptable salts.

In some embodiments, an additional therapeutic agent is, as a non-limiting example: xanthophyll.

In some embodiments, an additional therapeutic agent is, as a non-limiting example: A composition capable of preventing, delaying and/or decreasing any symptom of a retinopathy.

In some embodiments, an additional therapeutic agent is, as a non-limiting example: A siRNA. In some embodiments, an additional therapeutic agent is a siRNA delivered into the eye. In some embodiments, an oligonucleotide and/or an additional therapeutic agent is delivered by intravitreal injection.

In some embodiments, prior to the dose administration, a mydriatic (1% tropicamide) is instilled in the eye, followed by a topical anesthetic.

In some embodiments, an additional therapeutic agent is, as a non-limiting example: gene therapy.

In some embodiments, an additional therapeutic agent is, as a non-limiting example: encapsulating cells releasing a neurotrophic factor(s).

In some embodiments, an additional therapeutic agent is, as a non-limiting example: stem cell transplantation.

In some embodiments, an additional therapeutic agent is, as a non-limiting example: LEDGF1-326.

In some embodiments, an additional therapeutic agent is, as a non-limiting example: an inhibitor of mitochondrial mu-calpain.

In some embodiments, an additional therapeutic agent is, as a non-limiting example: a peptide inhibitor of mitochondrial mu-calpain.

In some embodiments, an additional therapeutic agent is, as a non-limiting example: Tat-mu CL (HIV-N mu).

In some embodiments, an additional therapeutic agent is, as a non-limiting example: Curcumin.

In some embodiments, an additional therapeutic agent is, as a non-limiting example: a chaperone.

In some embodiments, an additional therapeutic agent is, as a non-limiting example: Grp78/BiP.

In some embodiments, RP is associated with inflammation.

In some embodiments, an additional therapeutic agent is, as a non-limiting example: an agent which reduces inflammation.

In some embodiments, an additional therapeutic treatment is, as a non-limiting example: a method of editing an USH2A gene. In some embodiments, an additional therapeutic treatment is, as a non-limiting example: a method of editing an USH2A gene in a cell, comprising the steps of: introducing into the cell one or more DNA endonucleases to effect one or more single-strand or double-strand breaks within or near the USH2A gene that result(s) in permanent deletion of an expanded trinucleotide repeat in the USH2A gene or replacement of one or more nucleotide bases, or one or more exons and/or introns within or near the USH2A gene, thereby restoring the USH2A gene function.

In some embodiments, an additional therapeutic agent is, as a non-limiting example: An oligonucleotide.

In some embodiments, a second or additional therapeutic agent is administered to a subject prior, simultaneously with, or after, an USH2A oligonucleotide. In some embodiments, a second or additional therapeutic agent is administered multiple times to a subject, and an USH2A oligonucleotide is also administered multiple times to a subject, and the administrations are in any order.

In some embodiments, an improvement may include decreasing the expression, activity and/or level of a mutant gene transcript or gene product thereof which is too high in a disease state (e.g., the gene transcript of a mutant USH2A gene comprising a deleterious mutation or a gene product thereof); increasing the expression, activity and/or level of a wild-type gene transcript or gene product thereof which is too low in the disease state (e.g., an at least partially functional USH2A protein which is translated from an USH2A in which deleterious exon has been skipped).

In some embodiments, an USH2A oligonucleotide useful for treating, ameliorating and/or preventing an USH2A-related condition, disorder or disease can be administered (e.g., to a subject) via any method described herein or known in the art.

In some embodiments, provided oligonucleotides, e.g., USH2A oligonucleotides are administered as pharmaceutical composition, e.g., for treating, ameliorating and/or preventing USH2A-related conditions, disorders or diseases. In some embodiments, provided oligonucleotides comprise at least one chirally controlled internucleotidic linkage. In some embodiments, provided oligonucleotide compositions are chirally controlled.

In some cases, patients with Usher Syndrome (e.g., Usher Syndrome Type 2A), atypical Usher syndrome, or nonsyndromic retinitis pigmentosa reportedly can further suffer from an additional, associated disorder or disease or complication, such as deafness or blindness.

Patients with Usher syndrome type 2 reportedly have a moderate to severe hearing impairment from birth and commonly experience the first symptoms of night blindness in their second decade of life, which progresses to complete blindness by the third or fourth decade of life.

In some embodiments, an additional therapeutic agent includes a treatment for an additional, associated disorder or disease or complication.

In some embodiments, a particular USH2A oligonucleotide has a reduced capability of eliciting a side effect or adverse effect, compared to a different USH2A oligonucleotide.

In some embodiments, an additional therapeutic agent can be administered to the patient in order to control or alleviate one or more side effects or adverse effects associated with administration of an oligonucleotide.

In some embodiments, an oligonucleotide and one or more additional therapeutic agent are administered to a patient (in any order), wherein the additional therapeutic agent can be administered to the patient in order to control or alleviate one or more side effects or adverse effects associated with administration of the oligonucleotide.

In some embodiments, an oligonucleotide and one or more additional therapeutic agent are administered to a patient (in any order), wherein the additional therapeutic agent can be administered to the patient in order to control or alleviate one or more side effects or adverse effects associated with administration of the oligonucleotide, and wherein the oligonucleotide targets USH2A.

In some embodiments, an oligonucleotide and one or more additional therapeutic agent are administered to a patient (in any order), wherein the additional therapeutic agent can be administered to the patient in order to control or alleviate one or more side effects or adverse effects associated with administration of the USH2A oligonucleotide.

In some embodiments, an oligonucleotide and one or more additional therapeutic agent are administered to a patient (in any order), wherein the additional therapeutic agent can be administered to the patient in order to control or alleviate one or more side effects or adverse effects associated with administration of the USH2A oligonucleotide.

In some embodiments, an oligonucleotide composition and one or more additional therapeutic agent are administered to a patient (in any order), wherein the additional therapeutic agent can be administered to the patient in order to control or alleviate one or more side effects or adverse effects associated with administration of the oligonucleotide composition, and wherein the oligonucleotide composition is chirally controlled or comprises at least one chirally controlled internucleotidic linkage (including but not limited to a chirally controlled phosphorothioate).

### Administration of Oligonucleotides and Compositions Thereof

Many delivery methods, regimen, etc. can be utilized in accordance with the present disclosure for administering provided oligonucleotides and compositions thereof (typically pharmaceutical compositions for therapeutic purposes), including various technologies known in the art.

In some embodiments, an USH2A oligonucleotide is injected directly into the eye.

In some embodiments, an USH2A oligonucleotide (and, optionally, an additional therapeutic agent, is delivery to the eye or the retina or ear or inner ear or cochlea using any method, device or composition described herein or known in the art. Non-limiting examples of documents describing various methods, devices and compositions useful for delivering an USH2A oligonucleotide (and optionally, an additional therapeutic agent) to the eye or the retina include: patents and patent applications U.S. Pat. Nos. 6,416,777, 6,299,895, 5,725,493, 5,443,505, EP1473003, US20170073674, US20170173183, US20180169131, US20180250370, WO2018055134. In some embodiments, an USH2A oligonucleotide (and, optionally, an additional therapeutic agent, is delivery to the eye or the retina or ear or inner ear or cochlea using any method, device or composition described herein or known in the art, including but not limited to: a drug delivery device, an ophthalmic drug delivery device, a device and method for treating ophthalmic diseases, a method of intravitreal medicine delivery, or a biocompatible ocular implant. In some embodiments, delivery of an USH2A oligonucleotide (and, optionally, an additional therapeutic agent) to the retina is by injection of an USH2A oligonucleotide (and, optionally, an additional therapeutic agent) to the sub-retinal space of the retina. In some embodiments, an USH2A oligonucleotide (and, optionally, an additional therapeutic agent) are administered in one or more locations in the sub-retinal space of the retina. In some embodiments, systemic modes of administration of an USH2A oligonucleotide (and, optionally, an additional therapeutic agent) include oral and parenteral routes. In some embodiments, parenteral routes include, as non-limiting examples: intravenous, intraarterial, intramuscular, intradermal, subcutaneous, intranasal, and intraperitoneal routes. In some embodiments, an oligonucleotide or additional therapeutic agent administered systemically may be modified or formulated to target the an oligonucleotide and an optional additional therapeutic agent to the eye or inner ear. In some embodiments, local modes of administration of an USH2A oligonucleotide (and, optionally, an additional therapeutic agent) include, as non-limiting examples, intraocular, intraorbital, subconjunctival, intravitreal, subretinal, transscleral or intracochlear routes. In some embodiments, significantly smaller amounts of the USH2A oligonucleotide (and, optionally, an additional therapeutic agent) may exert an effect when administered locally (for example, intravitreally) compared to when administered systemically (for example, intravenously). In some embodiments, local modes of administration can reduce or eliminate the incidence of potentially toxic side effects that may occur when therapeutically effective amounts of a component are administered systemically. In some embodiments, an oligonucleotide and an optional additional therapeutic agent are delivered subretinally, e.g., by subretinal injection. In some embodiments, subretinal injections may be made directly into the macular, e.g., submacular injection. In some embodiments, an oligonucleotide and an optional additional therapeutic agent are delivered by intravitreal injection. In some embodiments, intravitreal injection reportedly has a relatively low risk of retinal detachment. In some embodiments, nanoparticle or viral, e.g., AAV vector, is delivered intravitreally. In some embodiments, an oligonucleotide and an optional additional therapeutic agent are delivered into the inner ear, e.g., by intracochlear injection. In some embodiments, intracochlear injections may be made in the vicinity of inner and/or outer hair cells. In some embodiments, methods for administration of agents to the eye and inner ear are known in the medical arts and can be used to administer an oligonucleotide and an optional additional therapeutic agent. Exemplary methods include intraocular injection (e.g., retrobulbar, subretinal, submacular, intravitreal and intrachoridal), iontophoresis, eye drops, intraocular implantation (e.g., intravitreal, sub-Tenons and subconjunctival) and intracochlear injection. In some embodiments, administration may be provided as a periodic bolus (for example, subretinally, intravenously, intravitreally or by intracochlear injection) or as continuous infusion from an internal reservoir (for example, from an implant disposed at an intra- or extra-ocular location (see, U.S. Pat. Nos. 5,443,505 and 5,766,242)) or from an external reservoir (for example, from an intravenous bag). Components may be administered locally, for example, by continuous release from a sustained release drug delivery device immobilized to an inner wall of the eye or via targeted transscleral controlled release into the choroid (see, for example, PCT/USOO/00207, PCT/US02/14279, Ambati et al. (2000) INVEST. OPHTHALMOL. VIS. SCI. 41: 1181-1185, and Ambati et al. (2000) INVEST. OPHTHALMOL. VIS. SCI. 41: 1186-1191). A variety of devices suitable for administering an oligonucleotide and an optional additional therapeutic agent locally to the inside of the eye are known in the art. See, for example, U.S. Pat. Nos. 6,251,090, 6,299,895, 6,416,777, 6,413,540, and PCT/USOO/28187. In some embodiments, an oligonucleotide and an optional additional therapeutic agent can be formulated to permit release over a prolonged period of time. In some embodiments, a release system can include a matrix of a biodegradable material or a material which releases the incorporated an oligonucleotide and an optional additional therapeutic agent by diffusion. In some embodiments, the an oligonucleotide and an optional additional therapeutic agent can be homogeneously or heterogeneously distributed within the release system. In some embodiments, a variety of release systems may be useful, however, the choice of the appropriate system will depend upon rate of release required by a particular application. In some embodiments, a non-degradable or degradable release systems is used. In some embodiments, suitable release systems include polymers and polymeric matrices, non-polymeric matrices, or inorganic and organic excipients and diluents such as, but not limited to, calcium carbonate and sugar (for example, trehalose). In some embodiments, release systems may be natural or synthetic. In some embodiments, the release system material can be selected so that an oligonucleotide and an optional additional therapeutic agent having different molecular weights are released by diffusion through or degradation of the material. In some embodiments, synthetic, biodegradable polymers include as non-limiting examples: polyamides such as poly(amino acids) and poly(peptides); polyesters such as poly(lactic acid), poly(glycolic acid), poly(lactic-co-glycolic acid), and poly(caprolactone); poly(anhydrides); polyorthoesters; polycarbonates; and chemical derivatives thereof (substitutions, additions of chemical groups, for example, alkyl, alkylene, hydroxylations, oxidations, and other modifications routinely made by those skilled in the art), copolymers and mixtures thereof. In some embodiments, synthetic, non-degradable polymers include, as non-limiting examples: polyethers such as poly(ethylene oxide), poly(ethylene glycol), and poly(tetramethylene oxide); vinyl polymers-polyacrylates and polymethacrylates such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; polysiloxanes; and any chemical derivatives thereof (substitutions, additions of chemical groups, for example, alkyl, alkylene, hydroxylations, oxidations, and other modifications routinely made by those skilled in the art), copolymers and mixtures thereof. In some embodiments, poly(lactide-co-glycolide) microsphere can also be used for intraocular injection. In some embodiments, the microspheres are composed of a polymer of lactic acid and glycolic acid, which are structured to form hollow spheres. In some embodiments, the spheres can be approximately 15-30 microns in diameter and can be loaded with an oligonucleotide and an optional additional therapeutic agent.

In some embodiments, an oligonucleotide is delivered by intravitreal injection. In some embodiments, prior to intravitreal injection, a mydriatic (e.g., 1% tropicamide) is instilled in the eye, followed by a topical anesthetic.

In some embodiments, an oligonucleotide composition, e.g., an USH2A oligonucleotide composition, is administered at a dose and/or frequency lower than that of an otherwise comparable reference oligonucleotide composition and has comparable or improved effects. In some embodiments, a chirally controlled oligonucleotide composition is administered at a dose and/or frequency lower than that of a comparable, otherwise identical stereorandom reference oligonucleotide composition and with comparable or improved effects, e.g., in improving the skipping of a deleterious exon in an USH2A gene transcript.

In some embodiments, the present disclosure recognizes that properties and activities, e.g., ability to mediate exon skipping, stability, toxicity, etc. of oligonucleotides and compositions thereof can be modulated and optimized by chemical modifications and/or stereochemistry. In some embodiments, the present disclosure provides methods for optimizing oligonucleotide properties and/or activities through chemical modifications and/or stereochemistry. In some embodiments, the present disclosure provides oligonucleotides and compositions thereof with improved properties and/or activities. Without wishing to be bound by any theory, due to, e.g., their better activity, stability, delivery, distribution, toxicity, pharmacokinetic, pharmacodynamics and/or efficacy profiles, Applicant notes that provided oligonucleotides and compositions thereof in some embodiments can be administered at lower dosage and/or reduced frequency to achieve comparable or better efficacy, and in some embodiments can be administered at higher dosage and/or increased frequency to provide enhanced effects.

In some embodiments, the present disclosure provides, in a method of administering an oligonucleotide composition comprising a plurality of oligonucleotides sharing a common base sequence, the improvement comprising administering an oligonucleotide comprising a plurality of oligonucleotides that is characterized by improved delivery relative to a reference oligonucleotide composition of the same common base sequence.

In some embodiments, provided oligonucleotides, compositions and methods provide improved delivery. In some embodiments, provided oligonucleotides, compositions and methods provide improved cytoplasmatic delivery. In some embodiments, improved delivery is to a population of cells. In some embodiments, improved delivery is to a tissue. In some embodiments, improved delivery is to an organ. In some embodiments, improved delivery is to an organism, e.g., a patient or subject. Example structural elements (e.g., chemical modifications, stereochemistry, combinations thereof, etc.), oligonucleotides, compositions and methods that provide improved delivery are extensively described in the present disclosure.

Various dosing regimens can be utilized to administer oligonucleotides and compositions of the present disclosure. In some embodiments, multiple unit doses are administered, separated by periods of time. In some embodiments, a given composition has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second (or subsequent) dose amount that is the same as or different from the first dose (or another prior dose) amount. In some embodiments, a dosing regimen comprises administering at least one unit dose for at least one day. In some embodiments, a dosing regimen comprises administering more than one dose over a time period of at least one day, and sometimes more than one day. In some embodiments, a dosing regimen comprises administering multiple doses over a time period of at least a week. In some embodiments, the time period is at least 2, 3, 4, 5, 6, 7, 8, 9, 10, 11, 12, 13, 14, 15, 16, 17, 18, 19, 20, 21, 22, 23 24, 25, 26, 27, 28, 29, 30, 31, 32, 33, 34, 35, 36, 37, 38, 39, 40 or more (e.g., about 45, 50, 55, 60, 65, 70, 75, 80, 85, 90, 95, 100 or more) weeks. In some embodiments, a dosing regimen comprises administering one dose per week for more than one week. In some embodiments, a dosing regimen comprises administering one dose per week for 2, 3, 4, 5, 6, 7, 8, 9, 10, 11, 12, 13, 14, 15, 16, 17, 18, 19, 20, 21, 22, 23 24, 25, 26, 27, 28, 29, 30, 31, 32, 33, 34, 35, 36, 37, 38, 39, 40 or more (e.g., about 45, 50, 55, 60, 65, 70, 75, 80, 85, 90, 95, 100 or more) weeks. In some embodiments, a dosing regimen comprises administering one dose every two weeks for more than two week period. In some embodiments, a dosing regimen comprises administering one dose every two weeks over a time period of 2, 3, 4, 5, 6, 7, 8, 9, 10, 11, 12, 13, 14, 15, 16, 17, 18, 19, 20, 21, 22, 23 24, 25, 26, 27, 28, 29, 30, 31, 32, 33, 34, 35, 36, 37, 38, 39, 40 or more (e.g., about 45, 50, 55, 60, 65, 70, 75, 80, 85, 90, 95, 100 or more) weeks. In some embodiments, a dosing regimen comprises administering one dose per month for one month. In some embodiments, a dosing regimen comprises administering one dose per month f or more than one month. In some embodiments, a dosing regimen comprises administering one dose per month for 2, 3, 4, 5, 6, 7, 8, 9, 10, 11, 12, or more months. In some embodiments, a dosing regimen comprises administering one dose per week for about 10 weeks. In some embodiments, a dosing regimen comprises administering one dose per week for about 20 weeks. In some embodiments, a dosing regimen comprises administering one dose per week for 26 weeks. In some embodiments, a dosing regimen comprises administering one dose per week for about 30 weeks. In some embodiments, a chirally controlled oligonucleotide composition is administered according to a dosing regimen that differs from that utilized for a non-chirally controlled (e.g., stereorandom) oligonucleotide composition of the same sequence, and/or of a different chirally controlled oligonucleotide composition of the same sequence. In some embodiments, a chirally controlled oligonucleotide composition is administered according to a dosing regimen that is reduced as compared with that of a chirally uncontrolled (e.g., stereorandom) oligonucleotide composition of the same sequence in that it achieves a lower level of total exposure over a given unit of time, involves one or more lower unit doses, and/or includes a smaller number of doses over a given unit of time. In some embodiments, a chirally uncontrolled oligonucleotide is administered according to a dosing regimen that extends for a longer period of time than does that of a chirally uncontrolled (e.g., stereorandom) oligonucleotide composition of the same sequence Without wishing to be limited by theory, Applicant notes that in some embodiments, the shorter dosing regimen, and/or longer time periods between doses, may be due to the improved stability, bioavailability, and/or efficacy of a chirally controlled oligonucleotide composition. In some embodiments, with their improved delivery (and other properties), provided compositions can be administered in lower dosages and/or with lower frequency to achieve biological effects, for example, clinical efficacy.

### Pharmaceutical Compositions

In some embodiments, the present disclosure provides pharmaceutical compositions comprising a provided compound, e.g., an oligonucleotide, or a pharmaceutically acceptable salt thereof, and a pharmaceutical carrier. In some embodiments, for therapeutic and clinical purposes, oligonucleotides of the present disclosure are provided as pharmaceutical compositions.

In some embodiments, the present disclosure pertains to an USH2A oligonucleotide in a pharmaceutical composition suitable for injection into the eye.

As appreciated by those skilled in the art, oligonucleotides of the present disclosure can be provided in their acid, base or salt forms. In some embodiments, oligonucleotides can be in acid forms, e.g., for natural phosphate linkages, in the form of —OP(O)(OH)O—; for phosphorothioate internucleotidic linkages, in the form of —OP(O)(SH)O—; etc. In some embodiments, provided oligonucleotides can be in salt forms, e.g., for natural phosphate linkages, in the form of —OP(O)(ONa)O— in sodium salts; for phosphorothioate internucleotidic linkages, in the form of —OP(O)(SNa)O— in sodium salts; etc. Unless otherwise noted, oligonucleotides of the present disclosure can exist in acid, base and/or salt forms.

When used as therapeutics, a provided oligonucleotide, e.g., an USH2A oligonucleotide, or oligonucleotide composition thereof is typically administered as a pharmaceutical composition. In some embodiments, a pharmaceutical composition is suitable for administration of an oligonucleotide to an area of a body affected by a condition, disorder or disease. In some embodiments, a pharmaceutical composition comprises a therapeutically effective amount of a provided oligonucleotide or a pharmaceutically acceptable salt thereof, and a pharmaceutically acceptable inactive ingredient. In some embodiments, a pharmaceutically acceptable inactive ingredient is selected from pharmaceutically acceptable diluents, pharmaceutically acceptable excipients, and pharmaceutically acceptable carriers. In some embodiments, a pharmaceutically acceptable inactive ingredient is a pharmaceutically acceptable carrier.

In some embodiments, a provided oligonucleotide is formulated for administration to and/or contact with a body cell and/or tissue expressing its target. For example, in some embodiments, a provided USH2A oligonucleotide is formulated for administration to a body cell and/or tissue expressing USH2A. In some embodiments, such a cell and/or tissue of the eye or ear or any other tissue which expresses USH2A. In some embodiments, broad distribution of oligonucleotides and compositions may be achieved with intraparenchymal administration, intrathecal administration, or intracerebroventricular administration.

In some embodiments, the pharmaceutical composition is formulated for intravenous injection, oral administration, buccal administration, inhalation, nasal administration, topical administration, ophthalmic administration or otic administration. In some embodiments, the pharmaceutical composition is a tablet, a pill, a capsule, a liquid, an inhalant, a nasal spray solution, a suppository, a suspension, a gel, a colloid, a dispersion, a suspension, a solution, an emulsion, an ointment, a lotion, an eye drop or an ear drop.

In some embodiments, the present disclosure provides a pharmaceutical composition comprising chirally controlled oligonucleotide or composition thereof, in admixture with a a pharmaceutically acceptable inactive ingredient (e.g., a pharmaceutically acceptable excipient, a pharmaceutically acceptable carrier, etc.). One of skill in the art will recognize that the pharmaceutical compositions include pharmaceutically acceptable salts of provided oligonucleotide or compositions. In some embodiments, a pharmaceutical composition is a chirally controlled oligonucleotide composition. In some embodiments, a pharmaceutical composition is a stereopure oligonucleotide composition.

In some embodiments, the present disclosure provides salts of oligonucleotides and pharmaceutical compositions thereof. In some embodiments, a salt is a pharmaceutically acceptable salt. In some embodiments, a pharmaceutical composition comprises an oligonucleotide, optionally in its salt form, and a sodium salt. In some embodiments, a pharmaceutical composition comprises an oligonucleotide, optionally in its salt form, and sodium chloride. In some embodiments, each hydrogen ion of an oligonucleotide that may be donated to a base (e.g., under conditions of an aqueous solution, a pharmaceutical composition, etc.) is replaced by a non-H⁺ cation. For example, in some embodiments, a pharmaceutically acceptable salt of an oligonucleotide is an all-metal ion salt, wherein each hydrogen ion (for example, of —OH, —SH, etc.) of each internucleotidic linkage (e.g., a natural phosphate linkage, a phosphorothioate internucleotidic linkage, etc.) is replaced by a metal ion. Various suitable metal salts for pharmaceutical compositions are widely known in the art and can be utilized in accordance with the present disclosure. In some embodiments, a pharmaceutically acceptable salt is a sodium salt. In some embodiments, a pharmaceutically acceptable salt is magnesium salt. In some embodiments, a pharmaceutically acceptable salt is a calcium salt. In some embodiments, a pharmaceutically acceptable salt is a potassium salt. In some embodiments, a pharmaceutically acceptable salt is an ammonium salt (cation N(R)₄⁺). In some embodiments, a pharmaceutically acceptable salt comprises one and no more than one types of cation. In some embodiments, a pharmaceutically acceptable salt comprises two or more types of cation. In some embodiments, a cation is Li⁺, Na⁺, K⁺, Mg²⁺ or Ca²⁺. In some embodiments, a pharmaceutically acceptable salt is an all-sodium salt. In some embodiments, a pharmaceutically acceptable salt is an all-sodium salt, wherein each internucleotidic linkage which is a natural phosphate linkage (acid form —O—P(O)(OH)—O—), if any, exists as its sodium salt form (—O—P(O)(ONa)—O—), and each internucleotidic linkage which is a phosphorothioate internucleotidic linkage linkage (acid form —O—P(O)(SH)—O—), if any, exists as its sodium salt form (—O—P(O)(SNa)—O—).

Various technologies for delivering nucleic acids and/or oligonucleotides are known in the art can be utilized in accordance with the present disclosure. For example, a variety of supramolecular nanocarriers can be used to deliver nucleic acids. Example nanocarriers include, but are not limited to liposomes, cationic polymer complexes and various polymeric compounds. Complexation of nucleic acids with various polycations is another approach for intracellular delivery; this includes use of PEGylated polycations, polyethyleneamine (PEI) complexes, cationic block co-polymers, and dendrimers. Several cationic nanocarriers, including PEI and polyamidoamine dendrimers help to release contents from endosomes. Other approaches include use of polymeric nanoparticles, microspheres, liposomes, dendrimers, biodegradable polymers, conjugates, prodrugs, inorganic colloids such as sulfur or iron, antibodies, implants, biodegradable implants, biodegradable microspheres, osmotically controlled implants, lipid nanoparticles, emulsions, oily solutions, aqueous solutions, biodegradable polymers, poly(lactide-coglycolic acid), poly(lactic acid), liquid depot, polymer micelles, quantum dots and lipoplexes. In some embodiments, an oligonucleotide is conjugated to another molecule.

In therapeutic and/or diagnostic applications, compounds, e.g., oligonucleotides, of the disclosure can be formulated for a variety of modes of administration, including systemic and topical or localized administration. Techniques and formulations generally may be found in Remington, The Science and Practice of Pharmacy (20th ed. 2000).

Provided oligonucleotides and compositions thereof are effective over a wide dosage range. For example, in the treatment of adult humans, dosages from about 0.01 to about 1000 mg, from about 0.5 to about 100 mg, from about 1 to about 50 mg per day, and from about 5 to about 100 mg per day are examples of dosages that may be used. Exact dosages may depend upon routes of administration, forms in which provided compounds, e.g., oligonucleotides, are administered, subjects to be treated, conditions, disorders or diseases to be treated, body weights of the subjects to be treated, and/or preferences and experiences of physicians.

Pharmaceutically acceptable salts for basic moieties are generally well known to those of ordinary skill in the art, and may include, e.g., acetate, benzenesulfonate, besylate, benzoate, bicarbonate, bitartrate, bromide, calcium edetate, carnsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington, The Science and Practice of Pharmacy (20th ed. 2000). Preferred pharmaceutically acceptable salts include, for example, acetate, benzoate, bromide, carbonate, citrate, gluconate, hydrobromide, hydrochloride, maleate, mesylate, napsylate, pamoate (embonate), phosphate, salicylate, succinate, sulfate, or tartrate.

In some embodiments, provided oligonucleotides are formulated in pharmaceutical compositions described in WO 2005/060697, WO 2011/076807 or WO 2014/136086.

Depending on the specific conditions, disorders or diseases being treated, provided agents, e.g., oligonucleotides, may be formulated into liquid or solid dosage forms and administered systemically or locally. Provided oligonucleotides may be delivered, for example, in a timed- or sustained- low release form as is known to those skilled in the art. Techniques for formulation and administration may be found in Remington, The Science and Practice of Pharmacy (20th ed. 2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or another mode of delivery.

For injection, provided agents, e.g., oligonucleotides may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulations. Such penetrants are generally known in the art and can be utilized in accordance with the present disclosure.

Use of pharmaceutically acceptable carriers to formulate compounds, e.g., provided oligonucleotides, for the practice of the disclosure into dosages suitable for various mods of administration is well known in the art. With proper choice of carrier and suitable manufacturing practice, compositions of the present disclosure, e.g., those formulated as solutions, may be administered via various routes, e.g., parenterally, such as by intravenous injection.

In some embodiments, a composition comprising an USH2A oligonucleotide further comprises any or all of: calcium chloride dihydrate, magnesium chloride hexahydrate, potassium chloride, sodium chloride, sodium phosphate dibasic anhydrous, sodium phosphate, monobasic dihydrate, and/or water for Injection. In some embodiments, a composition further comprises any or all of: calcium chloride dihydrate (0.21 mg) USP, magnesium chloride hexahydrate (0.16 mg) USP, potassium chloride (0.22 mg) USP, sodium chloride (8.77 mg) USP, sodium phosphate dibasic anhydrous (0.10 mg) USP, sodium phosphate monobasic dihydrate (0.05 m g) USP, and Water for Injection USP.

In some embodiments, a composition comprising an oligonucleotide further comprises any or all of: cholesterol, (6Z,9Z,28Z,31Z)-heptatriaconta-6,9,28,31-tetraen-19-yl-4-(dimethylamino) butanoate(DLin-MC3-DMA), 1,2-distearoyl-sn-glycero-3-phosphocholine (DSPC), alpha-(3′-{[1,2-di(myristyloxy)propanoxy] carbonylamino}propyl)-omega-methoxy, polyoxyethylene(PEG2000-C-DMG), potassium phosphate monobasic anhydrous NF, sodium chloride, sodium phosphate dibasic heptahydrate, and Water for Injection. In some embodiments, the pH of a composition comprising an USH2A oligonucleotide is -7.0. In some embodiments, a composition comprising an oligonucleotide further comprises any or all of: 6.2 mg cholesterol USP, 13.0 mg (6Z,9Z,28Z,31Z)-heptatriaconta-6,9,28,31-tetraen-19-yl-4-(dimethylamino) butanoate(DLin-MC3-DMA), 3.3 mg 1,2-distearoyl-sn-glycero-3-phosphocholine (DSPC), 1.6 mg α-(3′-{[1,2-di(myristyloxy)propanoxy]carbonylamino}propyl)-ω-methoxy, polyoxyethylene(PEG2000-C-DMG), 0.2 mg potassium phosphate monobasic anhydrous NF, 8.8 mg sodium chloride USP, 2.3 mg sodium phosphate dibasic heptahydrate USP, and Water for Injection USP, in an approximately 1 mL total volume.

Provided compounds, e.g., oligonucleotides, can be formulated readily using pharmaceutically acceptable carriers well known in the art into dosages suitable for oral administration. In some embodiments, such carriers enable provided oligonucleotides to be formulated as tablets, pills, capsules, liquids, gels, syrups, slurries, suspensions and the like, for, e.g., oral ingestion by a subject (e.g., patient) to be treated.

For nasal or inhalation delivery, provided compounds, e.g., oligonucleotides, may be formulated by methods known to those of skill in the art, and may include, e.g., examples of solubilizing, diluting, or dispersing substances such as saline, preservatives, such as benzyl alcohol, absorption promoters, and fluorocarbons.

In certain embodiments, oligonucleotides and compositions are delivered to the CNS. In certain embodiments, oligonucleotides and compositions are delivered to the cerebrospinal fluid. In certain embodiments, oligonucleotides and compositions are administered to the brain parenchyma. In certain embodiments, oligonucleotides and compositions are delivered to an animal/subject by intrathecal administration, or intracerebroventricular administration. Broad distribution of oligonucleotides and compositions may be achieved with methods of administration described herein and/or known in the art.

In certain embodiments, parenteral administration is by injection, by, e.g., a syringe, a pump, etc. In certain embodiments, an injection is a bolus injection. In certain embodiments, an injection is administered directly to a tissue or location, such as striatum, caudate, cortex, hippocampus and/or cerebellum.

In certain embodiments, methods of specifically localizing provided compounds, e.g., oligonucleotides, such as by bolus injection, may decrease median effective concentration (EC50) (e.g., concentration at which the oligonucleotide or oligonucleotide composition is capable of mediating 50% skipping of a deleterious exon in an USH2A transcript) by a factor of 20, 25, 30, 35, 40, 45 or 50. In certain embodiments, a targeted tissue is brain tissue. In certain embodiments, a targeted tissue is striatal tissue. In certain embodiments, decreasing EC50 is desirable because it reduces the dose required to achieve a pharmacological result in a patient in need thereof.

In certain embodiments, a provided oligonucleotide is delivered by injection or infusion once every month, every two months, every 90 days, every 3 months, every 6 months, twice a year or once a year.

Pharmaceutical compositions suitable for use in the present disclosure include compositions wherein the active ingredients, e.g., oligonucleotides, are contained in effective amounts to achieve their intended purposes. Determination of the effective amounts is well within the capability of those skilled in the art, especially in light of the detailed disclosure provided herein.

In addition to active ingredients, pharmaceutical compositions may contain suitable pharmaceutically acceptable carriers comprising excipients and auxiliaries which facilitate processing of an active compound into preparations which can be used pharmaceutically. Preparations formulated for oral administration may be in the form of tablets, dragees, capsules, or solutions.

In some embodiments, pharmaceutical compositions for oral use can be obtained by combining an active compound with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

In some embodiments, dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. Push-fit capsules can contain active ingredients, e.g., oligonucleotides, in admixture with fillers such as lactose, binders such as starches, and/or lubricants such as talc or magnesium stearate and, optionally, stabilizers. In soft capsules, active compounds, e.g., oligonucleotides, may be dissolved or suspended in suitable liquids, such as fatty oils, liquid paraffin, or liquid polyethylene glycols (PEGs). In addition, stabilizers may be added.

In some embodiments, a provided composition comprises a lipid. In some embodiments, a lipid is conjugated to an active compound, e.g., an oligonucleotide. In some embodiments, a lipid is not conjugated to an active compound. In some embodiments, a lipid comprises a C₁₀-C₄₀ linear, saturated or partially unsaturated, aliphatic chain. In some embodiments, a lipid comprises a C₁₀-C₄₀ linear, saturated or partially unsaturated, aliphatic chain, optionally substituted with one or more C₁₋₄ aliphatic group. In some embodiments, the lipid is selected from the group consisting of lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl alcohol. In some embodiments, an active compound is a provided oligonucleotide. In some embodiments, a composition comprises a lipid and an an active compound, and further comprises another component which is another lipid or a targeting compound or moiety. In some embodiments, a lipid is an amino lipid; an amphipathic lipid; an anionic lipid; an apolipoprotein; a cationic lipid; a low molecular weight cationic lipid; a cationic lipid such as CLinDMA and DLinDMA; an ionizable cationic lipid; a cloaking component; a helper lipid; a lipopeptide; a neutral lipid; a neutral zwitterionic lipid; a hydrophobic small molecule; a hydrophobic vitamin; a PEG-lipid; an uncharged lipid modified with one or more hydrophilic polymers; phospholipid; a phospholipid such as 1,2-dioleoyl-sn-glycero-3-phosphoethanolamine; a stealth lipid; a sterol; a cholesterol; a targeting lipid; or another lipid described herein or reported in the art suitable for pharmaceutical uses. In some embodiments, a composition comprises a lipid and a portion of another lipid capable of mediating at least one function of another lipid. In some embodiments, a targeting compound or moiety is capable of targeting a compound (e.g., an oligonucleotide) to a particular cell or tissue or subset of cells or tissues. In some embodiments, a targeting moiety is designed to take advantage of cell- or tissue-specific expression of particular targets, receptors, proteins, or another subcellular component. In some embodiments, a targeting moiety is a ligand (e.g., a small molecule, antibody, peptide, protein, carbohydrate, aptamer, etc.) that targets a composition to a cell or tissue, and/or binds to a target, receptor, protein, or another subcellular component.

Certain example lipids for delivery of an active compound, e.g., an oligonucleotide, allow (e.g., do not prevent or interfere with) the function of an active compound. In some embodiments, a lipid is lauric acid, myristic acid, palmitic acid, stearic acid, oleic acid, linoleic acid, alpha-linolenic acid, gamma-linolenic acid, docosahexaenoic acid (cis-DHA), turbinaric acid or dilinoleyl alcohol.

As described in the present disclosure, lipid conjugation, such as conjugation with fatty acids, may improve one or more properties of oligonucleotides.

In some embodiments, a composition for delivery of an active compound, e.g., an oligonucleotide, is capable of targeting an active compound to particular cells or tissues as desired. In some embodiments, a composition for delivery of an active compound is capable of targeting an active compound to a muscle cell or tissue. In some embodiments, the present disclosure provides compositions and methods related to delivery of active compounds, wherein the compositions comprise an active compound and a lipid. In various embodiments to a muscle cell or tissue, a lipid is selected from lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl alcohol.

In some embodiments, an USH2A oligonucleotide is delivered to the eye and/or ear, or a cell or tissue or portion thereof, via a delivery method or composition designed for delivery of nucleic acids to the eye or ear, or a cell or tissue or portion thereof.

In some embodiments, an USH2A oligonucleotide is delivered via a method or composition described in any of: Buyens et al. J. Control Release 2012, 158; 362-70; Couto L B, High K A. Viral vector-mediated RNA interference. Curr. Opin. Pharmacol. 2010, 5; 534-542; Gomes da Silva et al. Acc. Chem. Res. 2012, 45; 1163-71; Grijalvo et al. 2014 Expert Opinion on Therapeutic Patents 24(7); Montana et al. Bioconjug. Chem. 2007, 18; 302-8; Moshfeghi et al. Expert Opin. Investig. Drugs 2005, 14; 671-682; Muller et al. Curr. Drug Discov. Technol. 2011, 8; 207-27; Semple et al. Nat. Biotechnol. 2010, 28; 172-6; Soutschek et al. Nature 2004, 432; 173-8; Templeton N. Cationic liposomes as in vivo delivery vehicles. Biosci. Rep. 2002, 22; 283-95; Trabulo et al. Curr. Pharm. Des. 2013, 19; 2895-923; Troiber et al. Bioconjug. Chem. 2011, 22; 1737-52; Yousefi et al. J. Control Release 2013, 170; 209-18; Zhi et al. Bioconjug. Chem. 2013, 24; 487-519; Zhou et al. Pharmaceuticals 2013, 6; 85-107; Zimmermann et al. Nature 2006. 441; 111-4; Khorkova et al. Nature Biotechnology volume 35, pages 249-263 (2017); Kritika Goyal, Veena Koul, Yashveer Singh, and Akshay Anand, Central Nervous System Agents in Medicinal Chemistry 14, 2014, 43-59; Avinó et al. J Nucleic Acids. 2011; 2011: 586935; Passini et al. Sci. Transl. Med. 2011 Mar. 2; 3(72): 72ra18; Chen et al. Front. Neurosci. 30 Aug. 2017; Juliano et al. Nucleic Acids Research, Volume 44, Issue 14, 19 Aug. 2016, Pages 6518-6548; and/or any of the published patent applications: EP2822600; US 20090264506; US 20170080100; US20100144845; US 20180030443; US20100055168; US20100055169; US20100254901 US2010234282; US2010311654; US2011003754; US20110281787; US2012027861; US2012142765; US20122007795; US2012230938; US20130183379; US2013281658; U.S. Pat. No. 9,938,526; WO2010017328; WO2010039088; WO2010045584; WO2010056403; WO2010085665; WO2010088565; WO2010111466; WO2010129672 WO2010135207; WO2011005566; WO2011017456; WO2011022460; WO2011028850; WO2011045747; WO2011053989; WO2011055888; WO2011064552; WO2011109698; WO2011115555; WO2011115862; WO2011116152; WO2011120053; WO2011120953; WO2011126937 WO2011126974; WO2011135138; WO2011135141; WO2011143008; WO2011153120; WO2011163121; WO201153493; WO2012009448; WO2012024396; WO2012030745; WO2012044638; WO2012054365; WO2012061259; WO2012061402; WO2012068187; WO2012082574; WO2012089352; WO2012099755; WO2012101235; WO2012113846; WO2012119051; WO2012142480; WO2012150960; WO2012162210; WO2012173994; WO2012176138; WO2013016157; WO2013030569; WO2013032643; WO2013040295; WO2013044116; WO2013049328; WO2013070010; WO2013075035; WO2013082286; WO2013086207; WO2013086322; WO2013086354; WO2013101983; WO2013110679; WO2013110679; WO2013110680; WO2013116126; WO2013123217; WO2013126564; WO2013148541; WO2013148736; WO2013155493; WO2013158579; WO2013160773; WO2013166121; and/or WO2013170386.

In some embodiments, an USH2A oligonucleotide is delivered via a composition comprising any one or more of, or a method of delivery involving the use of any one or more of: transferrin receptor-targeted nanoparticle; cationic liposome-based delivery strategy; cationic liposome; polymeric nanoparticle; viral carrier; retrovirus; adeno-associated virus; stable nucleic acid lipid particle; polymer; cell-penetrating peptide; lipid; dendrimer; neutral lipid; cholesterol; lipid-like molecule; fusogenic lipid; hydrophilic molecule; polyethylene glycol (PEG) or a derivative thereof; shielding lipid; PEGylated lipid; PEG-C-DMSO; PEG-C-DMSA; DSPC; ionizable lipid; a guanidinium-based cholesterol derivative; ion-coated nanoparticle; metal-ion coated nanoparticle; manganese ion-coated nanoparticle; angubindin-1; nanogel; incorporation of the USH2A into a branched nucleic acid structure; and/or incorporation of the USH2A into a branched nucleic acid structure comprising 2, 3, 4 or more oligonucleotides.

In some embodiments, a composition comprising an oligonucleotide is lyophilized. In some embodiments, a composition comprising an oligonucleotide is lyophilized, and the lyophilized oligonucleotide is in a vial. In some embodiments, the vial is back filled with nitrogen. In some embodiments, the lyophilized oligonucleotide composition is reconstituted prior to administration. In some embodiments, the lyophilized oligonucleotide composition is reconstituted with a sodium chloride solution prior to administration. In some embodiments, the lyophilized oligonucleotide composition is reconstituted with a 0.9% sodium chloride solution prior to administration. In some embodiments, reconstitution occurs at the clinical site for administration. In some embodiments, in a lyophilized composition, an oligonucleotide composition is chirally controlled or comprises at least one chirally controlled internucleotidic linkage and/or the oligonucleotide targets USH2A.

### EXEMPLIFICATION

Certain examples of provided technologies (compounds (oligonucleotides, reagents, etc.), compositions, methods (methods of preparation, use, assessment, etc.), etc.) were presented herein.

### Example 1. Oligonucleotide Synthesis

Various technologies for preparing oligonucleotides and oligonucleotide compositions (both stereorandom and chirally controlled) are known and can be utilized in accordance with the present disclosure, including, for example, those in U.S. Pat. Nos. 9,394,333, 9,744,183, 9,605,019, 9,598,458, 9,982,257, 10,160,969, 10,479,995, US 2020/0056173, US 2018/0216107, US 2019/0127733, 10,450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612, the methods and reagents of each of which are incorporated herein by reference.

In some embodiments, oligonucleotides were prepared using suitable chiral auxiliaries, e.g., DPSE and/or PSM chiral auxiliaries. Various oligonucleotides, e.g., those in Table A1, and compositions thereof, were prepared in accordance with the present disclosure.

### Example 2. Example Procedures for Assessing Oligonucleotide Preparations

Various technologies can be used to assess the activity of USH2A oligonucleotides and compositions thereof. Certain technologies are described herein as examples.

Cells which can be used include various human and mouse cells.

In vitro assay methods: Weri-Rb-1 and Y79 cells (human retinoblastoma cell lines) were used for screening of USH2A Exon-13 skipping ASOs (USH2A oligonucleotides). Both cell lines were purchased from ATCC, cultured and maintained as suspension cultures using appropriate media suggested in the vendor protocols. Screening was performed in 96 WP formats, seeding about 20,000 cells per well and treating with specified concentrations of modified ASOs gymnotically (free uptake; no transfection reagents used). Cells were further incubated at 37 degree C. in a cell culture incubator for 48 hours before isolating the total RNA. Various experiments were carried out in biological duplicates. Total RNA was converted to cDNA as per vendors protocol and Taqman gene expression assays were used to quantify exon-13 skipped and un-skipped USH2A mRNA transcripts. Oligonucleotides were applied to Y79 and Weri-Rb1 cell lines with no delivery vehicle. 48 hours post treatment cells were harvested and RNA was isolated.

The skipping efficiency of the ASOs were calculated using the following formula:

$$Normalize for loading ( SRFS9 ) Skipping \% = \frac{( Skipped )}{( Skipped + Un - skipped )} \times 100$$

In some experiments, a negative control plasmid was used: WV-AE962, which does not target USH2A (non-USH2A-targeting). NTC, non-targeting control oligonucleotide.

Various USH2A oligonucleotides can be tested for their ability for skipping of a deleterious exon in an USH2A gene transcript.

### Example 3. Provided Technologies can Effectively Induce Skipping of Exon 13 in USH2A Gene Transcripts

Various technologies can be utilized to assess properties and/or activities of provided oligonucleotides and compositions thereof. Some such technologies are described in this Example. Those skilled in the art appreciate that many other technologies can be readily utilized. As demonstrated herein, provided oligonucleotides and compositions, among other things, can be highly active, e.g., in skipping an exon (e.g., exon 13) in an USH2A gene transcript.

Various USH2A oligonucleotides were designed and constructed. A number of USH2A oligonucleotides were tested, including testing capability of skipping of exon 13 in USH2A gene transcripts.

Various USH2A oligonucleotides described herein were constructed and tested for their ability to induce skipping of only exon 13 (productive skipping), or simultaneous skipping of exons 12 and 13 (non-productive skipping) of USH2A gene transcripts.

Various USH2A oligonucleotide compositions were assessed for their ability to induce skipping of exon 13 in an USH2A mRNA.

Various experimental protocols can be used to test the activity of USH2A oligonucleotides in vitro. Non-limiting examples of procedures which have been or which could be used to test the activity of USH2A oligonucleotides are described herein.

Cells, tissues, and organs which can be used include those which are human, mouse, non-human primate (NHP) or rabbit in origin.

In some experiments, the amount of skipping of an USH2A exon (e.g., exon 13) can be tested relative to no oligonucleotide or to a reference oligonucleotide (which differs from a tested oligonucleotide in any one or more of: stereochemistry, patterns of stereochemistry, chemical modification, patterns of chemical modification, base sequence, etc.). For some experiments, results of replicates can be shown.

As appreciated by those skilled in the art, in some embodiments, a reference assay, condition, compound, oligonucleotide, composition, etc., may be referred to as a comparator assay, condition, compound, oligonucleotide, composition, etc., respectively.

Various experiments were performed to evaluate the activity of certain oligonucleotides. Some results are shown in the following Tables. In some of these Tables, results of replicate experiments are shown; in some experiments, replicates are indicated by (1), (2), etc. In some of these Tables, not all controls may be shown (data not shown).

Various experiments were performed in retinoblastoma cells in vitro or in retinas ex vivo unless otherwise noted.

In some tables, wherein the ability of an oligonucleotide to skip exon 13 of USH2A (e.g., productive skipping) is tested: 100.0 would represent 100% skipping of exon 13 of USH2A, and 0.0 would represent 0% skipping of exon 13. In some tables, wherein the ability of an oligonucleotide to simultaneously skip exons 12 and 13 of USH2A (e.g., non-productive skipping) is tested: 100.0 would represent 100% simultaneous skipping of exons 12 and 13 of USH2A, and 0.0 would represent 0% simultaneous skipping of exons 12 and 13.

In some experiments in vitro, human retinoblastoma cells were used [Weri-Rb1 or Y-79 (Y79)]. These cells are commercially available.

In some experiments: RNA was isolated, and optimized Tagman probes were used for quantification. In some experiments, levels of skipped/unskipped USH2A gene transcripts were compared to SRFS9 gene transcript level as a comparator.

In vitro assay methods in some experiments: Weri-Rb-1 or Y79 cells (human retinoblastoma cell lines) were used for screening of USH2A Exon-13 skipping oligonucleotides. Both cell lines were purchased from ATCC, cultured and maintained as suspension cultures using appropriate media suggested in the vendor protocols. Screening was performed in 96 WP formats, seeding cells and treating them with specified concentrations of modified oligonucleotides gymnotically (free uptake; no transfection reagents used). Cells were further incubated at 37 degree C. in a cell culture incubator for 48 hours before isolating the total RNA. Various experiments were carried out in biological duplicates. Total RNA was converted to cDNA as per vendor protocol and Tagman gene expression assays were used to quantify exon-13 skipped and un-skipped USH2A mRNA transcripts. The skipping efficiency of the ASOs were calculated using the following formula:

$$Normalize for loading ( SRFS9 ) Skipping \% = \frac{( Skipped )}{( Skipped + Un - skipped )} \times 100$$

In some experiments with retinas ex vivo: Whole NHP (non-human primate) or human eyes were enucleated and immediately placed in DMEM 10% FBS, 1% pen strep. 15-24 hours post-enucleation, retinas were dissected into pieces of approximate equal size, added to a 96-well dish containing media described above, and treated with PBS or ASO for 48 hours. RNA was extracted and exon skipping efficiency was evaluated.

TABLE 1
Activity of certain oligonucleotides. Certain oligonucleotides
were tested for their efficacy in inducing skipping of exon 13
in an USH2A gene transcript in vitro. Oligonucleotides were tested
in retinoblastoma cells, at a concentration of 50 uM. In various
experiments: Numbers represent % exon 13 skipping, where 100.0
would represent 100.0% skipping and 0.0 would represent 0.0%
skipping. In various experiments, not all controls which were
performed are necessarily included in this disclosure.

| | |
|---|---|
| Oligonucleotide | Skipping |

| Mock | 0 | 0 |
|---|---|---|
| WV-AE962 | 0.0 | 0.0 |
| WV-20781 (1) | 52.6 | 51.8 |
| WV-20781 (2) | 49.9 | 56.9 |
| WV-21098 | 13.9 | 10.9 |
| WV-21099 | 23.8 | 22.8 |
| WV-21100 | 53.7 | 49.4 |
| WV-21105 (1) | 52.8 | 55.2 |
| WV-21105 (2) | 47.8 | 51.7 |

TABLE 2
Activity of certain oligonucleotides.
Certain oligonucleotides were tested for their efficacy in
inducing skipping of exon 13 in an USH2A gene transcript
in Y-79 retinoblastoma cells in vitro. Oligonucleotides
were tested in a single dose screening at a concentration
of 50 uM and delivered gymnotically. Oligonucleotide
WV-AE962 is a comparator which does not target USH2A.

| | |
|---|---|
| Oligonucleotide | Skipping |

| | | |
|---|---|---|
| Mock | 0 | 0 |
| WV-AE962 | 0.0 | 0.0 |
| WV-20781 | 52.6 | 51.8 |
| WV-20879 | 0.1 | 0.0 |
| WV-20880 | 6.1 | 5.4 |
| WV-20881 | 7.8 | 9.6 |
| WV-20882 | 31.6 | 29.9 |
| WV-20883 | 38.4 | 35.8 |
| WV-20884 | 30.0 | 33.2 |
| WV-20885 | 58.1 | 54.0 |
| WV-20886 | 32.8 | 32.0 |
| WV-20887 | 9.3 | 9.1 |
| WV-20888 | 23.9 | 22.4 |
| WV-20889 | 20.5 | 19.5 |
| WV-20890 | 42.7 | 49.0 |
| WV-20891 | 72.8 | 65.3 |
| WV-20892 | 70.7 | 74.7 |
| WV-20893 | 38.5 | 36.3 |
| WV-20894 | 18.7 | 17.8 |
| WV-20895 | 33.1 | 31.3 |
| WV-20896 | 39.0 | 42.7 |
| WV-20897 | 47.3 | 50.4 |
| WV-20898 | 30.2 | 31.3 |
| WV-20899 | 28.3 | 28.6 |
| WV-20900 | 28.9 | 29.2 |
| WV-20901 | 37.6 | 39.4 |
| WV-20902 | 90.8 | 88.3 |
| WV-20903 | 30.6 | 33.0 |
| WV-20904 | 33.0 | 35.7 |
| WV-20905 | 13.4 | 11.8 |
| WV-20906 | 23.5 | 27.5 |
| WV-20907 | 53.0 | 48.6 |
| WV-20908 | 68.5 | 66.3 |

TABLE 3
Activity of certain oligonucleotides.
Certain oligonucleotides were tested for their efficacy in
inducing skipping of exon 13 in an USH2A gene transcript
in Y-79 cells in vitro. Oligonucleotides were tested in
a single dose screening, at a concentration of 50 uM.

| | |
|---|---|
| Oligonucleotide | Skipping |

| | | |
|---|---|---|
| Mock | 0 | 0 |
| WV-AE962 | 0.0 | 0.0 |
| WV-20781 | 52.6 | 51.8 |
| WV-20909 | 48.7 | 45.3 |
| WV-20910 | 48.1 | 46.0 |
| WV-20911 | 0.4 | 0.3 |
| WV-20912 | 2.5 | 1.9 |
| WV-20913 | 24.2 | 23.7 |
| WV-20914 | 0.0 | 0.0 |
| WV-20915 | 0.0 | 0.0 |
| WV-20916 | 2.3 | 2.5 |
| WV-20917 | 11.0 | 13.5 |
| WV-20918 | 19.0 | 22.2 |
| WV-20919 | 0.2 | 0.1 |
| WV-20920 | 1.2 | 0.8 |
| WV-20921 | 0.3 | 0.3 |
| WV-20922 | 0.1 | 0.0 |
| WV-20923 | 7.7 | 9.9 |
| WV-20924 | 0.1 | 0.3 |
| WV-20925 | 0.2 | 0.1 |
| WV-20926 | 2.2 | 2.8 |
| WV-20927 | 0.0 | 0.0 |
| WV-20929 | 3.0 | 0.0 |
| WV-20930 | 0.3 | 0.2 |
| WV-20931 | 0.2 | 0.2 |
| WV-20932 | 0.0 | 0.2 |
| WV-20933 | 0.1 | 0.1 |
| WV-20934 | 0.0 | 0.1 |
| WV-20935 | 0.2 | 0.2 |
| WV-20936 | 0.1 | 0.1 |
| WV-20937 | 0.0 | 0.0 |
| WV-20938 | 0.2 | 0.1 |
| WV-20939 | 0.3 | 0.3 |

TABLE 4
Activity of certain oligonucleotides.
Certain oligonucleotides were tested for their efficacy in
inducing skipping of exon 13 in an USH2A gene transcript
in Y-79 cells in vitro. Oligonucleotides were tested in
a single dose screening, at a concentration of 50 uM.

| | |
|---|---|
| Oligonucleotide | Skipping |

| | | |
|---|---|---|
| Mock | 0 | 0 |
| WV-AE962 | 0.0 | 0.0 |
| WV-20781 | 52.6 | 51.8 |
| WV-20940 | 0.2 | 0.3 |
| WV-20941 | 0.3 | 0.4 |
| WV-20942 | 0.1 | 0.1 |
| WV-20943 | 0.4 | 0.1 |
| WV-20944 | 2.8 | 2.7 |
| WV-20945 | 0.6 | 0.9 |
| WV-20946 | 0.0 | 0.2 |
| WV-20947 | 0.0 | 0.0 |
| WV-20948 | 0.1 | 0.8 |
| WV-20949 | 0.5 | 0.4 |
| WV-20950 | 0.3 | 0.4 |
| WV-20951 | 0.9 | 1.4 |
| WV-20952 | 0.0 | 0.1 |
| WV-20953 | 0.0 | 0.0 |
| WV-20954 | 0.2 | 0.1 |
| WV-20955 | 0.1 | 0.2 |
| WV-20956 | 0.0 | 0.0 |
| WV-20957 | 0.6 | 0.6 |
| WV-20958 | 1.8 | 0.9 |
| WV-20959 | 0.8 | 1.0 |
| WV-20960 | 0.7 | 0.8 |
| WV-20961 | 0.2 | 0.1 |
| WV-20962 | 0.1 | 0.0 |
| WV-20963 | 0.2 | 0.1 |
| WV-20964 | 0.3 | 0.0 |
| WV-20965 | 0.6 | 0.8 |
| WV-20966 | 4.6 | 4.0 |
| WV-20967 | 0.1 | 0.2 |
| WV-20968 | 0.0 | 0.2 |
| WV-20969 | 0.0 | 0.0 |

TABLE 5
Activity of certain oligonucleotides.
Certain oligonucleotides were tested for their efficacy in
inducing skipping of exon 13 in an USH2A gene transcript
in Y-79 cells in vitro. Oligonucleotides were tested in
a single dose screening, at a concentration of 50 uM.

| | |
|---|---|
| Oligonucleotide | Skipping |

| | | |
|---|---|---|
| Mock | 0.0 | 0.0 |
| WV-AE962 | 0.0 | 0.0 |
| WV-20781 | 52.6 | 51.8 |
| WV-20970 | 0.1 | 0.0 |
| WV-20971 | 0.6 | 0.4 |
| WV-20972 | 0.3 | 0.3 |
| WV-20973 | 0.0 | 0.0 |
| WV-20974 | 0.1 | 0.1 |
| WV-20975 | 0.5 | 0.3 |
| WV-20976 | 1.9 | 1.3 |
| WV-20977 | 0.8 | 0.5 |
| WV-20978 | 0.0 | 0.0 |
| WV-20979 | 0.0 | 0.0 |
| WV-20980 | 1.2 | 1.4 |
| WV-20981 | 0.6 | 0.5 |
| WV-20982 | 0.2 | 0.2 |
| WV-20983 | 0.2 | 0.2 |
| WV-20984 | 0.1 | 0.1 |
| WV-20985 | 0.2 | 0.2 |
| WV-20986 | 0.5 | 0.4 |
| WV-20987 | 4.9 | 5.7 |
| WV-20988 | 60.4 | 60.8 |
| WV-20989 | 4.3 | 7.7 |
| WV-20990 | 12.8 | 15.3 |
| WV-20991 | 0.3 | 0.3 |
| WV-20992 | 0.7 | 0.8 |
| WV-20993 | 0.5 | 0.9 |
| WV-20994 | 0.6 | 0.9 |
| WV-20995 | 1.1 | 0.9 |
| WV-20996 | 0.7 | 0.4 |
| WV-20997 | 0.5 | 0.3 |
| WV-20998 | 0.0 | 0.0 |
| WV-20999 | 0.2 | 0.4 |

TABLE 6
Activity of certain oligonucleotides.
Certain oligonucleotides were tested for their efficacy in
inducing skipping of exon 13 in an USH2A gene transcript
in Y-79 cells in vitro. Oligonucleotides were tested in
a single dose screening, at a concentration of 50 uM.

| | |
|---|---|
| Oligonucleotide | Skipping |

| | | |
|---|---|---|
| Mock | 0 | 0 |
| WV-AE962 | 0.0 | 0.0 |
| WV-20781 | 52.6 | 51.8 |
| WV-21000 | 6.1 | 5.9 |
| WV-21001 | 4.6 | 4.1 |
| WV-21002 | 3.9 | 2.7 |
| WV-21003 | 10.9 | 9.7 |
| WV-21004 | 1.6 | 2.5 |
| WV-21005 | 4.0 | 3.6 |
| WV-21006 | 7.1 | 7.0 |
| WV-21007 | 5.0 | 5.0 |
| WV-21008 | 90.5 | 89.9 |
| WV-21009 | 9.5 | 7.2 |
| WV-21010 | 0.8 | 1.2 |
| WV-21011 | 1.3 | 0.6 |
| WV-21012 | 3.3 | 2.0 |

TABLE 7
Activity of certain oligonucleotides.
Certain oligonucleotides were tested for their efficacy in inducing skipping
of exon 13 in an USH2A gene transcript in retinoblastoma cells in vitro.
Oligonucleotides were tested at a concentration of 50 uM.

| | |
|---|---|
| Oligonucleotide | Skipping |

| | | |
|---|---|---|
| Mock | 0 | 0 |
| WV-AE962 | 0.0 | 0.0 |
| WV-20781 | 52.6 | 51.8 |
| WV-20882 | 31.6 | 29.9 |
| WV-20883 | 38.4 | 35.8 |
| WV-20884 | 30.0 | 33.2 |
| WV-20885 | 58.1 | 54.0 |
| WV-20886 | 32.8 | 32.0 |
| WV-20890 | 42.7 | 49.0 |
| WV-20891 | 72.8 | 65.3 |
| WV-20892 | 70.7 | 74.7 |
| WV-20893 | 38.5 | 36.3 |
| WV-20895 | 33.1 | 31.3 |
| WV-20896 | 39.0 | 42.7 |
| WV-20897 | 47.3 | 50.4 |
| WV-20898 | 30.2 | 31.3 |
| WV-20899 | 28.3 | 28.6 |
| WV-20900 | 28.9 | 29.2 |
| WV-20901 | 37.6 | 39.4 |
| WV-20902 | 90.8 | 88.3 |
| WV-20903 | 30.6 | 33.0 |
| WV-20904 | 33.0 | 35.7 |
| WV-20907 | 53.0 | 48.6 |
| WV-20908 | 68.5 | 66.3 |
| WV-20909 | 48.7 | 45.3 |
| WV-20910 | 48.1 | 46.0 |
| WV-20988 | 60.4 | 60.8 |
| WV-21008 | 90.5 | 89.9 |

TABLE 8
Activity of certain oligonucleotides.
Certain oligonucleotides were tested for their efficacy in inducing skipping of exon 13 in
an USH2A gene transcript in Y-79 cells in vitro. Oligonucleotides were tested at a concentration of 25,
8.33, 2.78, 0.93, 0.31, 0.10, or 0.03 uM.

| | WV-AE962 | WV-21100 | WV-21105 | WV-20781 | WV-20917 | WV-20885 |
|---|---|---|---|---|---|---|
| 25 uM | | | | | 0.9 | 18.6 |
| | 0.0 | | | | 0.6 | |
| 8.33 uM | | 5.5 | 5.5 | 5.8 | 0.3 | 5.8 |
| | | 4.9 | 5.8 | 5.2 | 0.3 | 5.6 |
| 2.78 uM | 0.1 | 3.2 | 2.4 | 2.5 | 0.1 | 2.3 |
| | | 2.4 | 2.1 | 1.9 | 0.1 | 2.1 |
| 0.93 uM | 0.2 | 1.6 | 0.9 | 0.9 | 0.0 | 1.0 |
| | | 1.4 | 0.8 | 0.7 | 0.0 | 0.8 |
| 0.31 uM | 0.0 | 0.4 | 0.3 | 0.3 | 0.0 | 0.4 |
| | | 0.4 | 0.4 | 0.3 | 0.0 | 0.2 |
| 0.10 uM | 0.0 | 0.2 | 0.2 | 0.1 | 0.0 | 0.1 |
| | 0.0 | 0.3 | 0.1 | 0.1 | 0.0 | 0.1 |
| 0.03 uM | 0.0 | 0.2 | 0.1 | 0.1 | 0.0 | 0.0 |
| | 0.0 | 0.2 | 0.1 | 0.1 | 0.0 | 0.1 |
| | WV-20891 | WV-20892 | WV-20902 | WV-20908 | WV-20988 | WV-21008 |
| 25 uM | 17.2 | 24.6 | 59.7 | 32.3 | 30.4 | 0.4 |
| | 15.6 | 22.5 | 66.2 | 31.0 | 19.7 | 0.7 |
| 8.33 uM | 4.7 | 5.6 | 41.8 | 9.3 | 13.4 | 2.0 |
| | 4.0 | 4.7 | 37.7 | 7.5 | 11.5 | |
| 2.78 uM | 2.0 | 2.0 | 15.3 | 2.8 | 5.7 | 0.2 |
| | 1.7 | 1.6 | 13.1 | 2.4 | 5.2 | 0.2 |
| 0.93 uM | 0.9 | 0.6 | 3.1 | 1.1 | 1.7 | |
| | 0.8 | 0.5 | 2.3 | 0.8 | 1.5 | 0.3 |
| 0.31 uM | 0.3 | 0.2 | 0.5 | 0.3 | 0.3 | |
| | 0.3 | 0.1 | 0.3 | 0.2 | 0.3 | 0.0 |
| 0.10 uM | 0.1 | 0.1 | 0.1 | 0.1 | 0.2 | 0.1 |
| | 0.1 | 0.1 | 0.1 | 0.1 | 0.1 | 0.0 |
| 0.03 uM | 0.1 | 0.0 | 0.1 | 0.1 | 0.1 | |
| | 0.1 | 0.0 | 0.1 | 0.0 | 0.1 | 0.0 |

TABLE 9
Activity of certain oligonucleotides.
Certain oligonucleotides were tested for their efficacy in inducing skipping of exon 13 in
an USH2A gene transcript in Y-79 cells in vitro. Oligonucleotides were tested at a concentration of 8.33,
2.78, 0.93, 0.31, 0.10, or 0.03 uM.

| | WV-AE962 | WV-21100 | WV-21105 | WV-20781 | WV-20917 | WV-20885 |
|---|---|---|---|---|---|---|
| 8.33 uM | | 5.5 | 5.5 | 5.8 | 0.3 | 5.8 |
| | | 4.9 | 5.8 | 5.2 | 0.3 | 5.6 |
| 2.78 uM | 0.1 | 3.2 | 2.4 | 2.5 | 0.1 | 2.3 |
| | | 2.4 | 2.1 | 1.9 | 0.1 | 2.1 |
| 0.93 uM | 0.2 | 1.6 | 0.9 | 0.9 | 0.0 | 1.0 |
| | | 1.4 | 0.8 | 0.7 | 0.0 | 0.8 |
| 0.31 uM | 0.0 | 0.4 | 0.3 | 0.3 | 0.0 | 0.4 |
| | | 0.4 | 0.4 | 0.3 | 0.0 | 0.2 |
| 0.10 uM | 0.0 | 0.2 | 0.2 | 0.1 | 0.0 | 0.1 |
| | 0.0 | 0.3 | 0.1 | 0.1 | 0.0 | 0.1 |
| 0.03 uM | 0.0 | 0.2 | 0.1 | 0.1 | 0.0 | 0.0 |
| | 0.0 | 0.2 | 0.1 | 0.1 | 0.0 | 0.1 |
| | WV-20891 | WV-20892 | WV-20902 | WV-20908 | WV-20988 | WV-21008 |
| 8.33 uM | 4.7 | 5.6 | 41.8 | 9.3 | 13.4 | 2.0 |
| | 4.0 | 4.7 | 37.7 | 7.5 | 11.5 | |
| 2.78 uM | 2.0 | 2.0 | 15.3 | 2.8 | 5.7 | 0.2 |
| | 1.7 | 1.6 | 13.1 | 2.4 | 5.2 | 0.2 |
| 0.93 uM | 0.9 | 0.6 | 3.1 | 1.1 | 1.7 | |
| | 0.8 | 0.5 | 2.3 | 0.8 | 1.5 | 0.3 |
| 0.31 uM | 0.3 | 0.2 | 0.5 | 0.3 | 0.3 | |
| | 0.3 | 0.1 | 0.3 | 0.2 | 0.3 | 0.0 |
| 0.10 uM | 0.1 | 0.1 | 0.1 | 0.1 | 0.2 | 0.1 |
| | 0.1 | 0.1 | 0.1 | 0.1 | 0.1 | 0.0 |
| 0.03 uM | 0.1 | 0.0 | 0.1 | 0.1 | 0.1 | |
| | 0.1 | 0.0 | 0.1 | 0.0 | 0.1 | 0.0 |

TABLE 10
Activity of certain oligonucleotides.
Certain oligonucleotides were tested for their efficacy in inducing simultaneous skipping
of exons 12 and 13 in an USH2A gene transcript in Y-79 cells in vitro. Oligonucleotides were tested
at a concentration of 25, 8.33, 2.78, 0.93, 0.31, 0.10, or 0.03 uM.

| | WV-AE962 | WV-21100 | WV-21105 | WV-20781 | WV-20917 | WV-20885 |
|---|---|---|---|---|---|---|
| 25 uM | 0.5 | | | | 3.0 | 2.5 |
| | 0.4 | | | | 2.9 | |
| 8.33 uM | 0.6 | 2.1 | 2.4 | 3.0 | 2.2 | 1.7 |
| | 0.4 | 2.1 | 2.6 | 2.3 | 2.2 | 1.7 |
| 2.78 uM | 0.6 | 1.7 | 1.8 | 1.6 | 1.4 | 1.4 |
| | 0.5 | 1.5 | 1.9 | 1.5 | 1.3 | 1.2 |
| 0.93 uM | 0.8 | 1.1 | 1.3 | 1.3 | 0.9 | 0.9 |
| | 0.6 | 1.3 | 1.1 | 1.1 | 0.9 | 0.9 |
| 0.31 uM | 0.8 | 1.1 | 1.0 | 1.0 | 0.9 | 0.9 |
| | 0.5 | 1.0 | 0.9 | 1.0 | 1.0 | 0.8 |
| 0.10 uM | 0.9 | 1.0 | 1.1 | 0.9 | 0.8 | 1.0 |
| | 0.6 | 0.9 | 1.0 | 0.9 | 0.9 | 0.8 |
| 0.03 uM | 0.7 | 1.0 | 0.9 | 0.9 | 1.0 | 0.8 |
| | 0.7 | 0.9 | 0.9 | 1.0 | 0.8 | 0.8 |
| | WV-20891 | WV-20892 | WV-20902 | WV-20908 | WV-20988 | WV-21008 |
| 25 uM | 3.5 | 3.8 | 12.7 | 4.2 | 9.4 | 7.8 |
| | 2.8 | 3.7 | 15.9 | 4.4 | 5.8 | 17.8 |
| 8.33 uM | 1.6 | 1.7 | 10.0 | 2.0 | 4.4 | 8.1 |
| | 1.6 | 1.6 | 9.3 | 1.8 | 5.2 | 15.8 |
| 2.78 uM | 1.4 | 1.1 | 4.3 | 1.3 | 2.9 | 9.5 |
| | 1.4 | 1.1 | 4.3 | 1.2 | 2.7 | 13.5 |
| 0.93 uM | 1.1 | 1.2 | 2.0 | 1.1 | 1.5 | 9.7 |
| | 1.0 | 1.0 | 2.1 | 0.9 | 1.4 | 13.2 |
| 0.31 uM | 1.1 | 0.8 | 1.0 | 0.9 | 1.1 | 11.3 |
| | 1.2 | 1.0 | 1.0 | 0.9 | 0.9 | 7.6 |
| 0.10 uM | 0.9 | 0.8 | 1.1 | 0.8 | 0.7 | 2.0 |
| | 0.8 | 0.8 | 0.9 | 1.1 | 0.8 | 5.8 |
| 0.03 uM | 0.9 | 0.9 | 0.8 | 0.8 | 0.8 | 2.3 |
| | 0.9 | 1.0 | 0.8 | 0.6 | 0.8 | 3.0 |

TABLE 11
Activity of certain oligonucleotides.
Certain oligonucleotides were tested for their efficacy in inducing
simultaneous skipping of exons 12 and 13 in an USH2A gene
transcript in Y-79 cells in vitro. Oligonucleotides were tested at a
concentration of 8.33, 2.78, 0.93, 0.31, 0.10, or 0.03 uM.

| | WV- | WV- | WV- | WV- | WV- | WV- |
|---|---|---|---|---|---|---|
| | AE962 | 21100 | 21105 | 20781 | 20917 | 20885 |
| 8.33 uM | 0.6 | 2.1 | 2.4 | 3.0 | 2.2 | 1.7 |
| | 0.4 | 2.1 | 2.6 | 2.3 | 2.2 | 1.7 |
| 2.78 uM | 0.6 | 1.7 | 1.8 | 1.6 | 1.4 | 1.4 |
| | 0.5 | 1.5 | 1.9 | 1.5 | 1.3 | 1.2 |
| 0.93 uM | 0.8 | 1.1 | 1.3 | 1.3 | 0.9 | 0.9 |
| | 0.6 | 1.3 | 1.1 | 1.1 | 0.9 | 0.9 |
| 0.31 uM | 0.8 | 1.1 | 1.0 | 1.0 | 0.9 | 0.9 |
| | 0.5 | 1.0 | 0.9 | 1.0 | 1.0 | 0.8 |
| 0.10 uM | 0.9 | 1.0 | 1.1 | 0.9 | 0.8 | 1.0 |
| | 0.6 | 0.9 | 1.0 | 0.9 | 0.9 | 0.8 |
| 0.03 uM | 0.7 | 1.0 | 0.9 | 0.9 | 1.0 | 0.8 |
| | 0.7 | 0.9 | 0.9 | 1.0 | 0.8 | 0.8 |
| | WV- | WV- | WV- | WV- | WV- | WV- |
| | 20891 | 20892 | 20902 | 20908 | 20988 | 21008 |
| 8.33 uM | 1.6 | 1.7 | 10.0 | 2.0 | 4.4 | 8.1 |
| | 1.6 | 1.6 | 9.3 | 1.8 | 5.2 | 15.8 |
| 2.78 uM | 1.4 | 1.1 | 4.3 | 1.3 | 2.9 | 9.5 |
| | 1.4 | 1.1 | 4.3 | 1.2 | 2.7 | 13.5 |
| 0.93 uM | 1.1 | 1.2 | 2.0 | 1.1 | 1.5 | 9.7 |
| | 1.0 | 1.0 | 2.1 | 0.9 | 1.4 | 13.2 |
| 0.31 uM | 1.1 | 0.8 | 1.0 | 0.9 | 1.1 | 11.3 |
| | 1.2 | 1.0 | 1.0 | 0.9 | 0.9 | 7.6 |
| 0.10 uM | 0.9 | 0.8 | 1.1 | 0.8 | 0.7 | 2.0 |
| | 0.8 | 0.8 | 0.9 | 1.1 | 0.8 | 5.8 |
| 0.03 uM | 0.9 | 0.9 | 0.8 | 0.8 | 0.8 | 2.3 |
| | 0.9 | 1.0 | 0.8 | 0.6 | 0.8 | 3.0 |

TABLE 12A
Activity of certain oligonucleotides.
Certain oligonucleotides were tested for their efficacy
in inducing simultaneous skipping of exons 12 and 13 in
an USH2A gene transcript in retinoblastoma cells in vitro.
Oligonucleotides were tested at a concentration of 8.33 uM.

| | | |
|---|---|---|
| Oligonucleotide | Exon 13 | Exon 12-13 |

| | | | | |
|---|---|---|---|---|
| WV-AE962 | | | 0.6 | 0.4 |
| WV-21100 | 5.5 | 4.9 | 2.1 | 2.1 |
| WV-21105 | 5.5 | 5.8 | 2.4 | 2.6 |
| WV-20781 | 5.8 | 5.2 | 3.0 | 2.3 |
| WV-20917 | 0.3 | 0.3 | 2.2 | 2.2 |
| WV-20885 | 5.8 | 5.6 | 1.7 | 1.7 |
| WV-20891 | 4.7 | 4.0 | 1.6 | 1.6 |
| WV-20892 | 5.6 | 4.7 | 1.7 | 1.6 |
| WV-20902 | 41.8 | 37.7 | 10.0 | 9.3 |
| WV-20908 | 9.3 | 7.5 | 2.0 | 1.8 |
| WV-20988 | 13.4 | 11.5 | 4.4 | 5.2 |
| WV-21008 | 2.0 | | 8.1 | 15.8 |

TABLE 12B
Activity of certain oligonucleotides.
This Table shows the relative amount of skipping of exon
13 (alone) / simultaneous skipping of exons 12 and 13.

| | Ratio of: | | Ratio of: |
|---|---|---|---|
| | Skipping exon 13 alone / | | Skipping exon 13 alone / |
| | Simultaneous skipping | | Simultaneous skipping |
| | of exons 12 and 13 | | of exons 12 and 13 |
| Oligonucleotide | (fold change) | Oligonucleotide | (fold change) |
| WV-AE962 | 0.0 | WV-20891 | 2.7 |
| WV-21100 | 2.5 | WV-20892 | 3.2 |
| WV-21105 | 2.3 | WV-20902 | 4.1 |
| WV-20781 | 2.1 | WV-20908 | 4.4 |
| WV-20917 | 0.1 | WV-20988 | 2.6 |
| WV-20885 | 3.4 | WV-21008 | 0.1 |

TABLE 13
Activity of certain oligonucleotides.
Certain oligonucleotides were tested for their efficacy in inducing
skipping of exon 13 in an USH2A gene transcript in Y-79 or
Weri-Rb1 cells in vitro. Oligonucleotides were tested at a
concentration of 25, 8.33, 2.78, 0.93, 0.31, 0.10, or 0.03 uM (uM).

| | Tests in Y-79 cells | | | | | Tests in Weri-Rb1 cells | | | |
|---|---|---|---|---|---|---|---|---|---|
| | WV- | WV- | WV- | WV- | WV- | WV- | WV- | WV- | WV- |
| | AE962 | 20781 | 20902 | 20908 | 20988 | 20781 | 20902 | 20908 | 20988 |
| 25 | | | 59.7 | 32.3 | 30.4 | | | | |
| uM | 0.0 | | 66.2 | 31.0 | 19.7 | | 91.3 | 46.5 | 68.6 |
| 8.33 | | 5.8 | 41.8 | 9.3 | 13.4 | | | | |
| uM | | 5.2 | 37.7 | 7.5 | 11.5 | 16.5 | 76.8 | 21.5 | 53.3 |
| 2.78 | 0.1 | 2.5 | 15.3 | 2.8 | 5.7 | | | | |
| uM | | 1.9 | 13.1 | 2.4 | 5.2 | 3.7 | 39.6 | 9.9 | 19.3 |
| 0.93 | 0.2 | 0.9 | 3.1 | 1.1 | 1.7 | | | | |
| uM | | 0.7 | 2.3 | 0.8 | 1.5 | 0.9 | 6.6 | 2.2 | 9.5 |
| 0.31 | 0.0 | 0.3 | 0.5 | 0.3 | 0.3 | | | | |
| uM | | 0.3 | 0.3 | 0.2 | 0.3 | 0.9 | 1.1 | 0.1 | 1.7 |
| 0.10 | 0.0 | 0.1 | 0.1 | 0.1 | 0.2 | | | | |
| uM | 0.0 | 0.1 | 0.1 | 0.1 | 0.1 | 0.8 | 0.0 | 0.0 | 0.1 |
| 0.03 | 0.0 | 0.1 | 0.1 | 0.1 | 0.1 | | | | |
| uM | 0.0 | 0.1 | 0.1 | 0.0 | 0.1 | 0.1 | | | |

TABLE 14
Activity of certain oligonucleotides.
Certain oligonucleotides were tested for their efficacy in inducing
skipping of exon 13 in an USH2A gene transcript in Y-79 cells in
vitro. Oligonucleotides were tested at a concentration of 20 uM,
and delivered gymnotically. In this and various other experiments,
WV-AE962 is a non-targeting control oligonucleotide.

| | |
|---|---|
| Oligonucleotide | Skipping |

| | | |
|---|---|---|
| H2O | 0 | 0 |
| WV-AE962 | 0 | 0 |
| WV-20902 | 82.0 | 91.9 |
| WV-24294 | 2.9 | |
| WV-24356 | 3.8 | 3.7 |
| WV-24365 | 18.5 | |
| WV-24374 | 18.3 | |
| WV-24295 | 34.9 | |
| WV-24357 | 25.9 | 36.4 |
| WV-24366 | 58.9 | |
| WV-24375 | 71.1 | 39.3 |
| WV-24296 | 11.6 | |
| WV-24358 | 9.0 | 34.1 |
| WV-24367 | 38.4 | 22.6 |
| WV-24376 | 68.7 | |
| WV-24297 | 67.0 | 73.1 |
| WV-24359 | 14.2 | 37.7 |
| WV-24368 | | 69.9 |
| WV-24377 | 44.9 | 54.3 |
| WV-20902 | 82.0 | 91.9 |
| WV-24360 | 62.2 | 74.8 |
| WV-24369 | 21.9 | 26.1 |
| WV-24378 | 53.5 | 28.5 |

TABLE 15
Activity of certain oligonucleotides.
Certain oligonucleotides were tested for their efficacy in
inducing skipping of exon 13 in an USH2A gene transcript
in Y-79 cells in vitro. Oligonucleotides were tested at
a concentration of 20 uM, and delivered gymnotically.

| | |
|---|---|
| Oligonucleotide | Skipping |

| | | |
|---|---|---|
| H2O | 0 | 0 |
| WV-AE962 | 0 | 0 |
| WV-20902 | 82.0 | 91.9 |
| WV-24360 | 62.2 | 74.8 |
| WV-24369 | 21.9 | 26.1 |
| WV-24378 | 53.5 | 28.5 |
| WV-24298 | 97.0 | 94.5 |
| WV-24361 | 65.9 | 26.4 |
| WV-24370 | 45.8 | 30.0 |
| WV-24379 | 59.6 | 18.2 |
| WV-24299 | 50.1 | 49.5 |
| WV-24362 | 17.7 | 24.8 |
| WV-24371 | 34.2 | |
| WV-24380 | 54.4 | |
| WV-24300 | 31.7 | 33.9 |
| WV-24363 | 14.0 | |
| WV-24372 | 46.0 | |
| WV-24381 | 60.7 | |
| WV-24301 | 11.7 | 17.6 |
| WV-24364 | 12.3 | |
| WV-24382 | 62.4 | 76.2 |

TABLE 16
Activity of certain oligonucleotides.
Certain oligonucleotides were tested for their efficacy in inducing skipping
of exon 13 in an USH2A gene transcript in Y-79 cells. Oligonucleotides
were tested at a concentration of 20 uM, and delivered gymnotically.

| | |
|---|---|
| Oligonucleotide | Skipping |

| | | |
|---|---|---|
| H2O | 0 | 0 |
| WV-AE962 | 0 | 0 |
| WV-24294 | 2.9 | |
| WV-24295 | 34.9 | |
| WV-24296 | 11.6 | |
| WV-24297 | 67.0 | 73.1 |
| WV-20902 | 82.0 | 91.9 |
| WV-24298 | 97.0 | 94.5 |
| WV-24299 | 50.1 | 49.5 |
| WV-24300 | 31.7 | 33.9 |
| WV-24301 | 11.7 | 17.6 |

TABLE 17
Activity of certain oligonucleotides.
Certain oligonucleotides were tested for their efficacy in
inducing skipping of exon 13 in an USH2A gene transcript
in Y-79 cells in vitro. Oligonucleotides were tested at
a concentration of 20 uM, and delivered gymnotically.

| | |
|---|---|
| Oligonucleotide | Skipping |

| | | |
|---|---|---|
| H2O | 0 | 0 |
| WV-AE962 | 0 | 0 |
| WV-20902 | 82.0 | 91.9 |
| WV-24356 | 3.8 | 3.7 |
| WV-24357 | 25.9 | 36.4 |
| WV-24358 | 9.0 | 34.1 |
| WV-24359 | 14.2 | 37.7 |
| WV-24360 | 62.2 | 74.8 |
| WV-24361 | 65.9 | 26.4 |
| WV-24362 | 17.7 | 24.8 |
| WV-24363 | 14.0 | |
| WV-24364 | 12.3 | |

TABLE 18
Activity of certain oligonucleotides.
Certain oligonucleotides were tested for their efficacy in
inducing skipping of exon 13 in an USH2A gene transcript
in Y-79 cells in vitro. Oligonucleotides were tested at
a concentration of 20 uM, and delivered gymnotically.

| | |
|---|---|
| Oligonucleotide | Skipping |

| | | |
|---|---|---|
| H2O | 0 | 0 |
| WV-AE962 | 0 | 0 |
| WV-20902 | 82.0 | 91.9 |
| WV-24365 | 18.5 | |
| WV-24366 | 58.9 | |
| WV-24367 | 38.4 | 22.6 |
| WV-24368 | | 69.9 |
| WV-24369 | 21.9 | 26.1 |
| WV-24370 | 45.8 | 30.0 |
| WV-24371 | 34.2 | |
| WV-24372 | 46.0 | |
| WV-24373 | | |

TABLE 19
Activity of certain oligonucleotides.
Certain oligonucleotides were tested for their efficacy in inducing
skipping of exon 13 in an USH2A gene transcript in Y-79 cells in
vitro. Oligonucleotides were tested at a concentration of 20 uM.

| | |
|---|---|
| Oligonucleotide | Skipping |

| | | |
|---|---|---|
| H2O | 0 | 0 |
| WV-AE962 | 0 | 0 |
| WV-20902 | 82.0 | 91.9 |
| WV-24374 | 18.3 | |
| WV-24375 | 71.1 | 39.3 |
| WV-24376 | 68.7 | |
| WV-24377 | 44.9 | 54.3 |
| WV-24378 | 53.5 | 28.5 |
| WV-24379 | 59.6 | 18.2 |
| WV-24380 | 54.4 | |
| WV-24381 | 60.7 | |
| WV-24382 | 62.4 | 76.2 |

TABLE 20
Activity of certain oligonucleotides.
Certain oligonucleotides were tested for their efficacy in
inducing skipping of exon 13 in an USH2A gene transcript
in Y-79 cells in vitro. Oligonucleotides were tested at
a concentration of 20 uM, and delivered gymnotically.

| | |
|---|---|
| Oligonucleotide | Skipping |

| | | |
|---|---|---|
| H2O | 0 | 0 |
| WV-AE962 | 0 | 0 |
| WV-20902 | 82.0 | 91.9 |
| WV-20917 | 28.1 | |
| WV-24394 | 4.4 | 7.4 |
| WV-24383 | 1.5 | 1.7 |
| WV-24384 | 3.6 | |
| WV-24385 | 7.9 | |
| WV-24386 | 9.1 | |
| WV-24387 | 9.4 | |
| WV-24388 | 1.3 | 1.5 |
| WV-24389 | 4.0 | 24.6 |

TABLE 21
Activity of certain oligonucleotides.
Certain oligonucleotides were tested for their efficacy in inducing
skipping of exon 13 in an USH2A gene transcript in Y-79 cells in
vitro. Oligonucleotides were tested at a concentration of 20 uM, and
delivered gymnotically.

| | |
|---|---|
| Oligonucleotide | Skipping |

| | | |
|---|---|---|
| H2O | 0 | 0 |
| WV-AE962 | 0 | 0 |
| WV-20902 | 82.0 | 91.9 |
| WV-24390 | 5.9 | 4.2 |
| WV-24391 | 4.3 | 9.8 |
| WV-24392 | 1.8 | 2.6 |
| WV-24393 | 20.8 | 21.8 |
| WV-24394 | 4.4 | 7.4 |
| WV-24395 | 8.2 | 9.6 |
| WV-24396 | 2.2 | |
| WV-24397 | 6.5 | 6.3 |

TABLE 22
Activity of certain oligonucleotides.
Certain oligonucleotides were tested for their efficacy in inducing
skipping of exon 13 in an USH2A gene transcript in Y-79 cells in
vitro. Oligonucleotides delivered gymnotically. Concentrations are
shown in a format of Conc. (log 10M).
EC50 for WV-20902 is 2.203 uM; for WV-20781 is 8.73 uM.

| | | |
|---|---|---|
| Conc. | WV-20902 | WV-20781 |

| | | | | |
|---|---|---|---|---|
| −1.39794 | 1.0 | 1.1 | 3.0 | 4.3 |
| −1 | 10.2 | 12.6 | 4.5 | 5.2 |
| −0.58503 | 28.6 | 23.9 | 5.0 | 4.5 |
| −0.19382 | 28.8 | 32.7 | 6.1 | 8.2 |
| 0.20412 | 49.4 | 44.2 | 17.9 | 15.9 |
| 0.60206 | 59.5 | 56.9 | 24.4 | 28.8 |
| 1 | 68.6 | 68.4 | 55.2 | 56.3 |
| 1.39794 | 87.3 | 81.6 | 74.0 | 75.3 |

TABLE 23A
Activity of certain oligonucleotides.
Certain oligonucleotides were tested for their efficacy in inducing
skipping of exon 12 in an USH2A gene transcript in NHP (non-human
primate) retina cultured ex vivo. Numbers represent amount of USH2A
Exon 11-13 skipping transcript (e.g., a USH2A transcript in which
exon 12 is skipped, thus joining exon 11 and exon 13) formed.
Oligonucleotides were tested at a concentration of 20, 10, 5, or
2.5 uM as indicated.

| | | | |
|---|---|---|---|
| PBS | 3.9 | WV-20902 (10 uM) | 84.7 |
| | 4.6 | | 84.2 |
| | 13.5 | | 63.2 |
| | 8.4 | | 63.7 |
| | 8.2 | | 91.3 |
| | 10.8 | | 91.5 |
| | 5.6 | | 67.2 |
| | 18.2 | | 66.8 |
| | 10.3 | | 67.5 |
| | 10.5 | | 68.6 |
| | 6.2 | | 68.8 |
| | 4.1 | | 68.0 |
| WV-AE962 | 3.1 | WV-20902 (5 uM) | 18.3 |
| | 5.4 | | 9.0 |
| | 20.1 | | 60.9 |
| | 12.0 | | 63.7 |
| | 7.3 | | 23.9 |
| | 7.0 | | 23.6 |
| | 6.1 | | 23.9 |
| | 11.0 | | 39.2 |
| | 10.7 | | 54.5 |
| | 10.5 | | 55.7 |
| | 10.8 | | 13.2 |
| | 11.2 | | 13.2 |
| WV-20902 | 79.2 | WV-20902 (2.5 uM) | 35.8 |
| (20 uM) | 79.3 | | 34.3 |
| | 83.6 | | 12.2 |
| | 83.4 | | 22.6 |
| | 88.6 | | 13.7 |
| | 88.3 | | 14.0 |
| | 74.0 | | |
| | 74.3 | | |
| | 73.4 | | |
| | 74.2 | | |
| | 73.7 | | |
| | 72.8 | | |

TABLE 23B
Activity of certain oligonucleotides.
Certain oligonucleotides were tested for their efficacy in inducing
skipping of exon 13 in an USH2A gene transcript in human retina
ex vivo. Numbers represent amount of USH2A Exon 12-14 skipping
transcript (e.g., a USH2A transcript in which exon 13 is skipped,
thus joining exon 12 and exon 14) formed. Oligonucleotides were
tested at a concentration of 20, 10, 5, or 2.5 uM as indicated
(respectively, _20, _10, _5 or _2.5); and delivery was gymnotic.

| | WV- | WV- | WV- | WV- | WV- | WV- | WV- |
|---|---|---|---|---|---|---|---|
| | AE962_ | 20781_ | 20781_ | 20781_ | 20902_ | 20902_ | 20902_ |
| PBS | 20 | 10 | 5 | 2.5 | 10 | 5 | 2.5 |
| 0 | 0.0 | 0.0 | 0.0 | 0.0 | 85.1 | 1.0 | 0.0 |
| 0 | 0.0 | 0.0 | 0.0 | 0.0 | 80.3 | 10.4 | 0.5 |
| 5.7 | 0.0 | 0.0 | 15.7 | 2.2 | 2.8 | 9.0 | 17.1 |
| 0 | 0.0 | 11.1 | 9.6 | 0.0 | 0.0 | 0.0 | 15.1 |
| 0 | 0.0 | 0.0 | 0.0 | | 20.7 | | 5.8 |

TABLE 24A
Activity of certain oligonucleotides.
Certain oligonucleotides were tested for their efficacy in inducing
skipping of exon 13 in an USH2A gene transcript in human retinas.
Oligonucleotides were tested at a concentration of 10 uM as indicated
(respectively, 20, _10, 5 or _2.5); and delivery was gymnotic.
12-14 indicates production of USH2A transcript wherein exon 12 is
joined directly to exon 14 (e.g., exon 13 is skipped); and 11-14
indicates production of USH2A transcript wherein exon 11 is joined
directly to exon 14 (e.g., exons 12 and 13 are simultaneously skipped).

| | | WV- | WV- | WV- | WV- | WV- | WV- |
|---|---|---|---|---|---|---|---|
| PBS | PBS | AE962_ | AE962_ | 20902_ | 20902_ | 20781_ | 20781_ |
| (12- | (11- | 20 | 20 | 10 | 10 | 10 | 10 |
| 14) | 14) | (12-14) | (11-14) | (12-14) | (11-14) | (12-14) | (11-14) |
| 0 | 2.6 | 0.0 | 3.5 | 85.1 | 6.2 | 0.0 | 3.6 |
| 0 | 3.6 | 0.0 | 3.1 | 80.3 | 13.2 | 0.0 | 4.4 |
| 5.7 | 2.6 | 0.0 | 3.4 | 2.8 | 5.8 | 0.0 | 2.9 |
| 0 | | 0.0 | 1.8 | 0.0 | 3.3 | 11.1 | 5.2 |
| 0 | | 0.0 | 4.3 | 20.7 | 8.3 | 0.0 | 3.4 |

TABLE 24B
Activity of certain oligonucleotides.
Certain oligonucleotides were tested for their efficacy in inducing
skipping of exon 13 alone, or simultaneous skipping of exons 12 and
13, in an USH2A gene transcript in human retinas. Oligonucleotides
were tested at a concentration of 10 uM and delivery was gymnotic.

| | |
|---|---|
| WV-20902 10 uM | 11.2 |
| | 10.5 |
| | 0.4 |
| | 0.0 |
| | 2.7 |
| WV-20781 10 uM | 0.0 |
| | 0.0 |
| | 0.0 |
| | 2.9 |
| | 0.0 |

TABLE 25A
Activity of certain oligonucleotides.
Certain oligonucleotides were tested for their efficacy in inducing
skipping of exon 13 in an USH2A gene transcript in Y-79
retinoblastoma cells in vitro. Oligonucleotides were tested at various
concentrations and delivered gymnotically. Oligonucleotide WV-
15962 (also referred to as WV-AE962) is a comparator which does
not target USH2A. Cells harvested and RNA isolated at 48 hours of
post treatment.

| Treatment | 0.15625 uM | | 0.3125 uM | | 0.625 uM | | 1.25 uM | |
|---|---|---|---|---|---|---|---|---|
| PBS* | | | | | | | | |
| WV- | 0.05 | 0.05 | 0.00 | 0.00 | 0.00 | 0.00 | 0.16 | 0.07 |
| 15962 | | | | | | | | |
| WV- | 2.61 | 2.79 | 10.03 | 6.76 | 15.67 | 15.25 | 35.20 | 34.56 |
| 20902 | | | | | | | | |
| Treatment | 2.5 uM | | 5 uM | | 10 uM | | 20 uM | |
| PBS* | | | | | | | | |
| WV- | 0.00 | 0.00 | 0.09 | 0.12 | 0.08 | 0.14 | 0.27 | 0.47 |
| 15962 | | | | | | | | |
| WV- | 46.74 | 47.29 | 69.73 | 65.54 | 82.88 | 83.52 | 93.38 | 93.03 |
| 20902 | | | | | | | | |

*Not detectable or no or insufficient samples.

TABLE 25B
Activity of certain oligonucleotides.
WV-20902 was tested for its efficacy in inducing
skipping of exon 13 in an USH2A gene transcript in
Y-79 retinoblastoma cells in vitro. EC50 is 2.534 uM.

| | |
|---|---|
| Conc. uM | % exon 13 skipping |

| | | |
|---|---|---|
| 0.16 | 2.61 | 2.79 |
| 0.31 | 10.03 | 6.76 |
| 0.63 | 15.67 | 15.25 |
| 1.25 | 35.20 | 34.56 |
| 2.50 | 46.74 | 47.29 |
| 5.00 | 69.73 | 65.54 |
| 10.00 | 82.88 | 83.52 |
| 20.00 | 93.38 | 93.03 |

TABLE 25C
Activity of certain oligonucleotides.
WV-30205 and WV-20781 were tested for their efficacy in inducing
skipping of exon 13 in an USH2A gene transcript in Y-79
retinoblastoma cells in vitro. Data from a set of results are
presented below.

| | EC50 (UM) |
|---|---|
| WV-20781 | 8.73 |
| WV-30205 | 4.11 |

TABLE 26
Activity of certain oligonucleotides.
Certain oligonucleotides were tested for their efficacy in inducing
skipping of exon 13 in an USH2A gene transcript in Y-79
retinoblastoma cells in vitro. Oligonucleotides were tested at various
concentrations and delivered gymnotically. Cells harvested and
RNA isolated at 48 hours of post treatment.

| Treatment | 0.04 | | 0.10 | | 0.26 | | 0.64 | |
|---|---|---|---|---|---|---|---|---|
| PBS* | | | | | | | | |
| WV-19802 | 2.82 | 3.64 | 0.70 | 0.59 | 0.17 | 0.87 | 1.27 | 1.30 |
| WV-20781 | 3.00 | 4.31 | 4.47 | 5.20 | 5.01 | 4.48 | 6.06 | 8.17 |
| WV-20902 | 1.01 | 1.06 | 10.19 | 12.60 | 28.65 | 23.93 | 28.84 | 32.71 |
| WV-24360 | 1.47 | 1.79 | 0.86 | 1.65 | 0.42 | 2.50 | 6.83 | 4.37 |
| WV-24298 | 1.40 | 1.46 | 1.17 | 0.98 | 5.78 | 7.35 | 9.47 | 8.66 |
| WV-30205 | 1.60 | 2.90 | 2.37 | 2.72 | 7.22 | 7.02 | 17.93 | 15.00 |
| Treatment | 1.60 | | 4.00 | | 10.00 | | 25.00 | |
| PBS* | | | | | | | | |
| WV-19802 | 2.47 | 3.36 | 1.26 | 1.37 | 2.65 | 3.40 | 2.39 | 2.95 |
| WV-20781 | 17.92 | 15.87 | 24.39 | 28.82 | 55.19 | 56.29 | 73.99 | 75.31 |
| WV-20902 | 49.44 | 44.18 | 59.52 | 56.94 | 68.64 | 68.39 | 87.33 | 81.64 |
| WV-24360 | 27.71 | 20.61 | 42.72 | 51.05 | 66.75 | 67.87 | 80.52 | 76.88 |
| WV-24298 | 14.02 | 12.94 | 24.71 | 17.64 | 53.13 | 62.90 | 77.30 | 76.91 |
| WV-30205 | 34.85 | 25.99 | 53.63 | 47.31 | 76.52 | 66.51 | 84.35 | 70.87 |

*Not detectable or no or insufficient samples.

TABLE 27
Activity of certain oligonucleotides.
Certain oligonucleotides were tested for their efficacy in inducing skipping
of exon 13 in an USH2A gene transcript in Y-79 retinoblastoma cells in
vitro. Oligonucleotides were tested at various concentrations and delivered
gymnotically. Cells harvested and RNA isolated at 48 hours of post treatment.

| Conc. | | | | | | | | | | |
|---|---|---|---|---|---|---|---|---|---|---|
| uM | WV-20902 | | WV-24360 | | WV-20781 | | WV-24298 | | WV-30205 | |
| 0.04 | 1.01 | 1.06 | 1.47 | 1.79 | 3.00 | 4.31 | 1.40 | 1.46 | 1.60 | 2.90 |
| 0.10 | 10.19 | 12.60 | 0.86 | 1.65 | 4.47 | 5.20 | 1.17 | 0.98 | 2.37 | 2.72 |
| 0.25 | 28.65 | 23.93 | 0.42 | 2.50 | 5.01 | 4.48 | 5.78 | 7.35 | 7.22 | 7.02 |
| 0.63 | 28.84 | 32.71 | 6.83 | 4.37 | 6.06 | 8.17 | 9.47 | 8.66 | 17.93 | 15.00 |
| 1.60 | 49.44 | 44.18 | 27.71 | 20.61 | 17.92 | 15.87 | 14.02 | 12.94 | 34.85 | 25.99 |
| 4.00 | 59.52 | 56.94 | 42.72 | 51.05 | 24.39 | 28.82 | 24.71 | 17.64 | 53.63 | 47.31 |
| 10.00 | 68.64 | 68.39 | 66.75 | 67.87 | 55.19 | 56.29 | 53.13 | 62.90 | 76.52 | 66.51 |
| 25.00 | 87.33 | 81.64 | 80.52 | 76.88 | 73.99 | 75.31 | 77.30 | 76.91 | 84.35 | 70.87 |

TABLE 28
Activity of certain oligonucleotides.
Certain oligonucleotides were tested for their efficacy in inducing
skipping of exon 13 in an USH2A gene transcript in Y-79
retinoblastoma cells in vitro. Oligonucleotides were tested at
various concentrations and delivered gymnotically. Cells
harvested and RNA isolated at 3; 24; 48 and 72 hours of post
treatment (No media change). Skipping % evaluated based on
PCR quantification assays.

| Time | | | WV-20902 | | WV-20902 | | WV-20902 | |
|---|---|---|---|---|---|---|---|---|
| point | PBS | | (5 uM) | | (10 uM) | | (20 um) | |
| 3 hours | 0.29 | 0.36 | 0.56 | 0.52 | 0.34 | 0.42 | 0.52 | 0.55 |
| 24 hours | 0.07 | 0.08 | 1.21 | 1.32 | 2.21 | 1.97 | 7.04 | 6.39 |
| 48 hours | 0.17 | 0.18 | 20.72 | 19.16 | 65.65 | 47.41 | 81.56 | 79.70 |
| 72 hours | 0.14 | 0.15 | 33.58 | 30.62 | 56.96 | 57.49 | 78.80 | 79.01 |

TABLE 29
Activity of certain oligonucleotides.
Certain oligonucleotides were tested for their efficacy in inducing
skipping of exon 13 alone, or simultaneous skipping of exons 12
and 13, in an USH2A gene transcript in human retinas.
Oligonucleotides were tested at various concentrations and
delivery was gymnotic.

| | WV- | | | | | |
|---|---|---|---|---|---|---|
| | 19802 | WV-20902 | | WV-24360 | | |
| PBS | 20 uM | 20 uM | 10 uM | 20 uM | 10 uM | 5 uM |
| 1.80 | 0.00 | 87.46 | 88.17 | 85.33 | 60.92 | 5.82 |
| 1.52 | 0.00 | 86.74 | 86.05 | 88.96 | 73.05 | 15.45 |
| 0.00 | 0.00 | 86.71 | 78.58 | 84.56 | 63.95 | 46.42 |
| 0.00 | 0.00 | 85.57 | 78.20 | 85.94 | 70.80 | 35.53 |
| 0.00 | 0.00 | 82.35 | 88.59 | 80.80 | 54.48 | |
| | 0.00 | 87.78 | 88.91 | 83.53 | 71.70 | |
| | 0.00 | 89.39 | 36.73 | 83.32 | | |
| | 0.00 | 86.66 | 39.38 | 88.72 | | |
| | 0.00 | 89.81 | | 88.69 | | |
| | 0.00 | 89.44 | | 89.31 | | |

| | WV-30205 | | WV-20781 | | |
|---|---|---|---|---|---|
| PBS | 20 uM | 10 uM | 20 uM | 10 uM | 5 uM |
| 1.80 | 59.95 | 46.41 | 83.37 | 0.00 | 20.63 |
| 1.52 | 63.18 | 51.44 | 82.21 | 0.00 | 26.95 |
| 0.00 | 60.79 | 35.05 | 88.66 | 0.00 | 0.00 |
| 0.00 | 60.88 | 40.77 | 89.68 | 0.00 | 0.00 |
| 0.00 | 64.18 | 39.39 | 87.15 | 46.00 | 30.36 |
| | 64.59 | 46.54 | 87.77 | 54.00 | 26.48 |
| | 86.66 | 46.06 | 82.27 | | 13.29 |
| | 77.28 | 47.69 | 83.91 | | |
| | 82.84 | | | | |
| | 89.15 | | | | |

TABLE 30
Activity of certain oligonucleotides.
Certain oligonucleotides were tested for their efficacy in
inducing skipping of exon 12 in an USH2A gene transcript
in NHP (non-human primate) retina cultured ex vivo.
Numbers represent amount of USH2A Exon 11-13
skipping transcript (e.g., a USH2A transcript in which
exon 12 is skipped, thus joining exon 11 and exon 13)
formed. Oligonucleotides were tested at a concentration
of 20, 10, 5, or 1 uM as indicated.

| Treatment | uM | Values = % exon 12 skipping | | | | | |
|---|---|---|---|---|---|---|---|
| PBS | | 3.88 | 4.63 | 13.50 | 8.39 | 8.22 | 10.79 |
| WV-15962 | 20 | 3.09 | 5.44 | 20.06 | 11.95 | 7.28 | 7.03 |
| WV-20781 | 20 | 51.24 | 49.97 | 60.22 | 61.46 | 49.18 | 46.84 |
| WV-24298 | 20 | 75.93 | 75.53 | 74.63 | 74.25 | 78.21 | 78.49 |
| WV-24360 | 20 | 44.96 | 44.39 | 77.34 | 77.15 | 86.07 | 85.74 |
| WV-24382 | 20 | 55.11 | 52.96 | 55.31 | 54.30 | 58.97 | 60.31 |
| WV-20902 | 20 | 79.25 | 79.31 | 83.60 | 83.36 | 88.55 | 88.30 |
| | 10 | 84.72 | 84.17 | 63.15 | 63.67 | 91.31 | 91.50 |
| | 5 | 18.31 | 8.98 | 60.91 | 63.71 | 23.87 | 23.60 |
| | 1 | 35.77 | 34.30 | 12.20 | 22.59 | 13.73 | 14.02 |
| PBS | | 5.55 | 18.15 | 10.31 | 10.52 | 6.24 | 4.08 |
| WV-15962 | 20 | 6.07 | 11.04 | 10.66 | 10.54 | 10.79 | 11.17 |
| WV-20781 | 20 | 65.74 | 67.70 | 56.64 | 61.34 | 24.73 | 29.85 |
| WV-24298 | 20 | 66.43 | 66.96 | 73.01 | 74.29 | 78.37 | 77.84 |
| WV-24360 | 20 | 77.79 | 77.97 | 76.62 | 76.42 | 89.59 | 90.17 |
| WV-24382 | 20 | 65.74 | 62.80 | 53.63 | 54.46 | 71.74 | 69.93 |
| WV-20902 | 20 | 73.96 | 74.29 | 73.38 | 74.20 | 73.70 | 72.82 |
| | 10 | 67.21 | 66.76 | 67.51 | 68.56 | 68.77 | 67.98 |
| | 5 | 23.91 | 39.16 | 54.50 | 55.69 | 13.17 | 13.23 |
| | 1 | | | | | | |

TABLE 31
Activity of certain oligonucleotides.
Certain oligonucleotides were tested for their efficacy in
inducing skipping of exon 12 in an USH2A gene transcript
in NHP (non-human primate) retina cultured ex vivo.
Numbers represent amount of USH2A Exon 11-13 skipping
transcript (e.g., a USH2A transcript in which exon 12 is skipped,
thus joining exon 11 and exon 13) formed. Oligonucleotides
were tested at a concentration of 20, 10, 5, or 1 uM as indicated.

| Treatment | uM | Values = % exon 12 skipping | | | | | | | |
|---|---|---|---|---|---|---|---|---|---|
| PBS | | 0.00 | 0.00 | 0.00 | 0.01 | 0.00 | 0.00 | 0.00 | 0.00 |
| WV-19802 | 20 | 0.00 | 0.00 | 0.00 | 0.00 | 0.00 | 0.00 | 0.01 | 0.00 |
| WV-20781 | 0.3 | 0.00 | 0.00 | 0.01 | 0.01 | 0.00 | 0.00 | 0.00 | 0.00 |
| | 1 | 2.98 | 4.05 | 3.09 | 3.57 | 3.11 | 3.44 | 3.26 | 3.38 |
| | 3 | 6.77 | 6.79 | 8.57 | 7.76 | 7.18 | 7.66 | 7.56 | 8.04 |
| | 10 | 13.71 | 16.48 | 16.91 | 20.89 | 16.98 | 19.63 | 17.22 | 18.33 |
| | 20 | 29.55 | 28.76 | 30.74 | 31.02 | 29.61 | 33.37 | 29.94 | 31.62 |
| WV-30205 | 0.3 | 0.00 | 0.00 | 0.00 | 0.00 | 0.00 | 0.00 | 0.00 | 0.00 |
| | 1 | 10.68 | 9.90 | 11.12 | 11.11 | 11.90 | 11.65 | 11.83 | 9.74 |
| | 3 | 35.37 | 36.53 | 37.85 | 40.71 | 41.03 | 37.87 | 38.93 | 38.23 |
| | 10 | 68.68 | 70.15 | 69.75 | 65.71 | 66.28 | 67.87 | 68.14 | 66.59 |
| | 20 | 78.68 | 82.20 | 79.96 | 80.05 | 81.34 | 81.55 | 80.04 | 80.43 |

TABLE 32
Activity of certain oligonucleotides.
Certain oligonucleotides were tested for their efficacy in inducing
skipping of exon 13 in an USH2A gene transcript in Y-79
retinoblastoma cells in vitro. Oligonucleotides were tested at various
concentrations and delivered gymnotically. Cells harvested and
RNA isolated at 48 hours of post treatment.

| | uM | | | | | | | |
|---|---|---|---|---|---|---|---|---|
| | 0 | | 0.3125 | | 0.625 | | 1.25 | |
| WV-19802 | 0.01 | 0.00 | 0.01 | 0.00 | 0.16 | 0.03 | 0.10 | 0.00 |
| WV-30205 | 0.87 | 0.69 | 2.83 | 2.63 | 5.58 | 5.06 | 10.36 | 11.60 |
| WV-36864 | 0.73 | 0.92 | 2.12 | 2.49 | 3.35 | 3.25 | 10.64 | 9.80 |
| WV-36865 | 0.63 | 0.69 | 1.99 | 2.78 | 3.44 | 3.56 | 7.89 | 7.51 |
| WV-36866 | 0.58 | 0.68 | 2.11 | 2.05 | 3.56 | 3.54 | 8.84 | 8.65 |
| WV-36867 | 1.56 | 1.74 | 4.51 | 5.05 | 5.83 | 7.52 | 12.61 | 13.57 |
| WV-36868 | 0.43 | 0.60 | 2.60 | 2.23 | 3.29 | 3.26 | 7.69 | 10.02 |
| | 2.5 | | 5 | | 10 | | 20 | |
| WV-19802 | 0.07 | 0.07 | 0.15 | 0.07 | 0.13 | 0.11 | 0.05 | 0.10 |
| WV-30205 | 17.12 | 8.02 | 27.94 | 28.92 | 41.62 | 41.17 | 67.38 | 65.41 |
| WV-36864 | 17.56 | 16.30 | 33.38 | 34.13 | 51.38 | 47.03 | 77.41 | 70.13 |
| WV-36865 | 13.40 | 13.38 | 24.09 | 24.83 | 37.58 | 35.52 | 60.43 | 61.97 |
| WV-36866 | 12.62 | 13.74 | 27.54 | 29.40 | 46.42 | 49.63 | 74.71 | 75.73 |
| WV-36867 | 21.34 | 22.92 | 41.38 | 40.96 | 62.42 | 59.82 | 82.20 | 81.77 |
| WV-36868 | 16.66 | 16.72 | 32.49 | 31.22 | 32.32 | 30.05 | 76.62 | 77.75 |

TABLE 33
Activity of certain oligonucleotides.
Certain oligonucleotides were tested for their efficacy in inducing
skipping of exon 13 in an USH2A gene transcript in Y-79 retinoblastoma
cells in vitro. Oligonucleotides were tested at various concentrations and
delivered gymnotically. Cells harvested and RNA isolated at 48 hours
of post treatment.

| | uM | | | | | | | |
|---|---|---|---|---|---|---|---|---|
| | 0 | | 0.3125 | | 0.625 | | 1.25 | |
| WV-19802 | 0.01 | 0.00 | 0.01 | 0.00 | 0.16 | 0.03 | 0.10 | 0.00 |
| WV-30205 | 0.87 | 0.69 | 2.83 | 2.63 | 5.58 | 5.06 | 10.36 | 11.60 |
| WV-33863 | 1.40 | 1.31 | 2.23 | 2.06 | 2.35 | 2.61 | 6.37 | 7.34 |
| WV-36437 | 0.69 | 0.20 | 0.98 | 0.97 | 0.73 | 0.66 | 4.08 | 5.05 |
| WV-36439 | 0.69 | 1.00 | 1.83 | 1.00 | 4.48 | 5.08 | 10.32 | 11.67 |
| | 2.5 | | 5 | | 10 | | 20 | |
| WV-19802 | 0.07 | 0.07 | 0.15 | 0.07 | 0.13 | 0.11 | 0.05 | 0.10 |
| WV-30205 | 17.12 | 8.02 | 27.94 | 28.92 | 41.62 | 41.17 | 67.38 | 65.41 |
| WV-33863 | 13.64 | 14.59 | 23.33 | 24.67 | 36.70 | 36.85 | 52.58 | 53.13 |
| WV-36437 | 12.46 | 10.36 | 19.18 | 19.47 | 29.90 | 30.80 | 46.92 | 48.06 |
| WV-36439 | 20.75 | 20.58 | 22.77 | 27.01 | 53.30 | 54.62 | 71.31 | 71.62 |

TABLE 34A
Activity of certain oligonucleotides.
Certain oligonucleotides were tested for their efficacy in inducing
skipping of exon 13 in an USH2A gene transcript in Y-79
retinoblastoma cells in vitro. Oligonucleotides were tested at
various concentrations and delivered gymnotically. Cells
harvested and RNA isolated at 48 hours of post treatment.

| | 25 uM | | 8.33 uM | | 2.78 uM | | 0.93 uM | |
|---|---|---|---|---|---|---|---|---|
| WV-15962 | | 0.01 | | | 0.06 | | 0.18 | |
| WV-20781 | | | 5.76 | 5.15 | 2.54 | 1.88 | 0.85 | 0.71 |
| WV-20902 | 59.66 | 66.24 | 41.80 | 37.73 | 15.27 | 13.12 | 3.14 | 2.35 |
| WV-20908 | 32.28 | 31.01 | 9.33 | 7.52 | 2.82 | 2.36 | 1.13 | 0.78 |
| WV-20988 | 30.36 | 19.72 | 13.40 | 11.47 | 5.68 | 5.22 | 1.69 | 1.53 |

| | 0.31 uM | | 0.10 uM | | 0.03 uM | |
|---|---|---|---|---|---|---|
| WV-15962 | 0.03 | | 0.04 | 0.01 | 0.00 | 0.00 |
| WV-20781 | 0.29 | 0.26 | 0.08 | 0.10 | 0.06 | 0.06 |
| WV-20902 | 0.49 | 0.32 | 0.12 | 0.11 | 0.07 | 0.07 |
| WV-20908 | 0.27 | 0.24 | 0.07 | 0.10 | 0.07 | 0.05 |
| WV-20988 | 0.34 | 0.29 | 0.18 | 0.09 | 0.10 | 0.09 |

TABLE 34B
Activity of certain oligonucleotides.
Certain oligonucleotides were tested for their efficacy in inducing
skipping of exon 13 in an USH2A gene transcript in Weri-Rb1 cells
in vitro. Oligonucleotides were tested at various concentrations.

| | 25 | 8.33 | 2.78 | 0.93 | 0.31 | 0.10 | 0.03 |
|---|---|---|---|---|---|---|---|
| | uM | uM | uM | uM | uM | uM | uM |
| WV-15962 | | | | | | | |
| WV-20781 | | 16.48 | 3.69 | 0.86 | 0.86 | 0.80 | 0.07 |
| WV-20902 | 91.32 | 76.79 | 39.61 | 6.61 | 1.05 | 0.00 | |
| WV-20908 | 46.45 | 21.47 | 9.87 | 2.16 | 0.12 | 0.01 | |
| WV-20988 | 68.60 | 53.29 | 19.32 | 9.52 | 1.71 | 0.14 | |

TABLE 35A
Activity of certain oligonucleotides.
Certain oligonucleotides were tested for their efficacy in inducing
skipping of exon 13 in an USH2A gene transcript in Y-79 retinoblastoma
cells in vitro. Oligonucleotides were tested a concentration of 5 uM
and delivered gymnotically. Cells harvested and RNA isolated at 48
hours of post treatment.

5 uM

| | | |
|---|---|---|
| PBS | 6.64 | 0.00 |
| WV-19802 | 0.00 | 0.00 |
| WV-20902 | 46.33 | 33.23 |
| WV-20988 | 31.68 | 15.60 |
| WV-32019 | 18.44 | 5.39 |
| WV-32020 | 22.03 | 12.54 |
| WV-32021 | 3.31 | 10.38 |
| WV-32022 | 6.45 | 25.59 |
| WV-20988 | 31.68 | 15.60 |
| WV-32023 | 26.69 | 23.72 |
| WV-32024 | 8.34 | 9.78 |
| WV-32025 | 29.26 | 8.72 |
| WV-32026 | 0.00 | 0.00 |

TABLE 35B
Activity of certain oligonucleotides.
Certain oligonucleotides were tested for their efficacy in inducing
skipping of exon 13 in an USH2A gene transcript in Y-79 retinoblastoma
cells in vitro. Oligonucleotides were tested a concentration of 5 uM
and delivered gymnotically. Cells harvested and RNA isolated at 48
hours of post treatment.

5 uM

| | | |
|---|---|---|
| PBS | 6.64 | 0.00 |
| WV-19802 | 0.00 | 0.00 |
| WV-20902 | 46.33 | 33.23 |
| WV-20988 | 31.68 | 15.60 |
| WV-32027 | 5.37 | 4.69 |
| WV-32028 | 3.99 | 2.85 |
| WV-32029 | 45.54 | 18.29 |
| WV-32030 | 14.22 | 22.05 |
| WV-32031 | 26.51 | 6.97 |
| WV-32032 | 15.40 | 10.68 |
| WV-32033 | 15.24 | 35.30 |
| WV-32034 | 23.34 | 15.86 |
| WV-32035 | 0.00 | 0.00 |

TABLE 35C
Activity of certain oligonucleotides.
Certain oligonucleotides were tested for their efficacy in inducing
skipping of exon 13 in an USH2A gene transcript in Y-79 retinoblastoma
cells in vitro. Oligonucleotides were tested a concentration of 5 uM
and delivered gymnotically. Cells harvested and RNA isolated at 48
hours of post treatment.

5 uM

| | | |
|---|---|---|
| PBS | 6.64 | 0.00 |
| WV-19802 | 0.00 | 0.00 |
| WV-20902 | 46.33 | 33.23 |
| WV-20988 | 31.68 | 15.60 |
| WV-32036 | 7.41 | 16.80 |
| WV-32037 | 2.04 | 2.04 |
| WV-32038 | 9.67 | 6.80 |
| WV-32039 | 3.78 | 7.33 |
| WV-32040 | 6.75 | 6.70 |
| WV-32041 | 9.74 | 23.15 |
| WV-32042 | 4.81 | 11.15 |
| WV-32043 | 1.30 | 1.82 |
| WV-32044 | 29.35 | 30.04 |

TABLE 35D
Activity of certain oligonucleotides.
Certain oligonucleotides were tested for their efficacy in inducing
skipping of exon 13 in an USH2A gene transcript in Y-79 retinoblastoma
cells in vitro. Oligonucleotides were tested a concentration of 5 uM
and delivered gymnotically. Cells harvested and RNA isolated at 48
hours of post treatment.

5 uM

| | | |
|---|---|---|
| PBS | 6.64 | 0.00 |
| WV-19802 | 0.00 | 0.00 |
| WV-20902 | 46.33 | 33.23 |
| WV-20988 | 31.68 | 15.60 |
| WV-32045 | 14.81 | 6.56 |
| WV-32046 | 24.96 | 10.46 |
| WV-32047 | 2.94 | 11.17 |
| WV-32048 | | |
| WV-32049 | 15.22 | 10.37 |
| WV-32050 | 18.47 | 16.54 |
| WV-32051 | 21.36 | 8.55 |
| WV-32052 | 3.36 | 2.24 |
| WV-32053 | 8.68 | 0.00 |

TABLE 36A
Activity of certain oligonucleotides.
Certain oligonucleotides were tested for their efficacy in inducing
skipping of exon 13 in an USH2A gene transcript in Y-79 retinoblastoma
cells in vitro. Oligonucleotides were tested a concentration of 5 uM
and delivered gymnotically. Cells harvested and RNA isolated at 48
hours of post treatment.

10 uM

| | | |
|---|---|---|
| PBS | 5.26 | 13.97 |
| WV-19802 | 13.97 | 5.53 |
| WV-20902 | 62.21 | 57.42 |
| WV-20988 | 74.24 | 51.99 |
| WV-32019 | 27.05 | 47.80 |
| WV-32020 | 15.70 | 35.39 |
| WV-32021 | 36.34 | 64.33 |
| WV-32022 | 39.33 | 34.95 |
| WV-20988 | 74.24 | 51.99 |
| WV-32023 | 49.23 | 62.35 |
| WV-32024 | 26.31 | 12.80 |
| WV-32025 | 39.78 | 47.12 |
| WV-32026 | 0.00 | 0.00 |

TABLE 36B
Activity of certain oligonucleotides.
Certain oligonucleotides were tested for their efficacy in inducing
skipping of exon 13 in an USH2A gene transcript in Y-79 retinoblastoma
cells in vitro. Oligonucleotides were tested a concentration of 5 uM
and delivered gymnotically. Cells harvested and RNA isolated at 48
hours of post treatment.

10 uM

| | | |
|---|---|---|
| PBS | 5.26 | 13.97 |
| WV-19802 | 13.97 | 5.53 |
| WV-20902 | 62.21 | 57.42 |
| WV-20988 | 74.24 | 51.99 |
| WV-32027 | 73.01 | 66.73 |
| WV-32028 | 64.50 | 47.29 |
| WV-32029 | 54.16 | 71.48 |
| WV-32030 | 65.86 | 68.62 |
| WV-32031 | 64.51 | 44.10 |
| WV-32032 | 33.37 | 12.49 |
| WV-32033 | 66.03 | 58.60 |
| WV-32034 | 30.68 | 58.87 |
| WV-32035 | 0.00 | 0.00 |

TABLE 36C
Activity of certain oligonucleotides.
Certain oligonucleotides were tested for their efficacy in inducing
skipping of exon 13 in an USH2A gene transcript in Y-79 retinoblastoma
cells in vitro. Oligonucleotides were tested a concentration of 5 uM
and delivered gymnotically. Cells harvested and RNA isolated at 48
hours of post treatment.

10 uM

| | | |
|---|---|---|
| PBS | 5.26 | 13.97 |
| WV-19802 | 13.97 | 5.53 |
| WV-20902 | 62.21 | 57.42 |
| WV-20988 | 74.24 | 51.99 |
| WV-32036 | 68.27 | 44.27 |
| WV-32037 | 26.71 | 48.31 |
| WV-32038 | 46.32 | 64.65 |
| WV-32039 | 9.80 | 15.99 |
| WV-32040 | 21.60 | 26.63 |
| WV-32041 | 43.86 | 32.81 |
| WV-32042 | 20.80 | 12.06 |
| WV-32043 | 54.41 | 40.97 |
| WV-32044 | 53.08 | 44.44 |

TABLE 36D
Activity of certain oligonucleotides.
Certain oligonucleotides were tested for their efficacy in inducing
skipping of exon 13 in an USH2A gene transcript in Y-79 retinoblastoma
cells in vitro. Oligonucleotides were tested a concentration of 5 uM
and delivered gymnotically. Cells harvested and RNA isolated at 48
hours of post treatment.

10 uM

| | | |
|---|---|---|
| PBS | 5.26 | 13.97 |
| WV-19802 | 13.97 | 5.53 |
| WV-20902 | 62.21 | 57.42 |
| WV-20988 | 74.24 | 51.99 |
| WV-32045 | 40.04 | 25.36 |
| WV-32046 | 59.67 | 48.76 |
| WV-32047 | 34.59 | 25.65 |
| WV-32048 | 0.00 | 0.00 |
| WV-32049 | 50.06 | 69.27 |
| WV-32050 | 46.22 | 38.16 |
| WV-32051 | 50.78 | 64.33 |
| WV-32052 | 4.13 | 21.13 |
| WV-32053 | 60.92 | 65.76 |

TABLE 37
Activity of certain oligonucleotides.
Certain oligonucleotides were tested for their efficacy in inducing
skipping of exon 13 in an USH2A gene transcript in Y-79 retinoblastoma
cells in vitro. Oligonucleotides were tested concentrations of 10 uM
and 5 uM and delivered gymnotically. Cells harvested and RNA isolated
at 48 hours of post treatment.

| | |
|---|---|
| 10 uM | 5 uM |

| | | | | |
|---|---|---|---|---|
| PBS | 5.26 | 13.97 | 6.64 | 0.00 |
| WV-19802 | 13.97 | 5.53 | 0.00 | 0.00 |
| WV-20902 | 62.21 | 57.42 | 46.33 | 33.23 |
| WV-20988 | 74.24 | 51.99 | 31.68 | 15.60 |
| WV-32019 | 27.05 | 47.80 | 18.44 | 5.39 |
| WV-32020 | 15.70 | 35.39 | 22.03 | 12.54 |
| WV-32021 | 36.34 | 64.33 | 3.31 | 10.38 |
| WV-32022 | 39.33 | 34.95 | 6.45 | 25.59 |
| WV-32023 | 49.23 | 62.35 | 26.69 | 23.72 |
| WV-32024 | 26.31 | 12.80 | 8.34 | 9.78 |
| WV-32025 | 39.78 | 47.12 | 29.26 | 8.72 |
| WV-32027 | 73.01 | 66.73 | 5.37 | 4.69 |
| WV-32028 | 64.50 | 47.29 | 3.99 | 2.85 |
| WV-32029 | 54.16 | 71.48 | 45.54 | 18.29 |
| WV-32030 | 65.86 | 68.62 | 14.22 | 22.05 |
| WV-32031 | 64.51 | 44.10 | 26.51 | 6.97 |
| WV-32032 | 33.37 | 12.49 | 15.40 | 10.68 |
| WV-32033 | 66.03 | 58.60 | 15.24 | 35.30 |
| WV-32034 | 30.68 | 58.87 | 23.34 | 15.86 |
| WV-32036 | 68.27 | 44.27 | 7.41 | 16.80 |
| WV-32037 | 26.71 | 48.31 | 2.04 | 2.04 |
| WV-32038 | 46.32 | 64.65 | 9.67 | 6.80 |
| WV-32039 | 9.80 | 15.99 | 3.78 | 7.33 |
| WV-32040 | 21.60 | 26.63 | 6.75 | 6.70 |
| WV-32041 | 43.86 | 32.81 | 9.74 | 23.15 |
| WV-32042 | 20.80 | 12.06 | 4.81 | 11.15 |
| WV-32043 | 54.41 | 40.97 | 1.30 | 1.82 |
| WV-32044 | 53.08 | 44.44 | 29.35 | 30.04 |
| WV-32045 | 40.04 | 25.36 | 14.81 | 6.56 |
| WV-32046 | 59.67 | 48.76 | 24.96 | 10.46 |
| WV-32047 | 34.59 | 25.65 | 2.94 | 11.17 |
| WV-32049 | 50.06 | 69.27 | 15.22 | 10.37 |
| WV-32050 | 46.22 | 38.16 | 18.47 | 16.54 |
| WV-32051 | 50.78 | 64.33 | 21.36 | 8.55 |
| WV-32052 | 4.13 | 21.13 | 3.36 | 2.24 |
| WV-32053 | 60.92 | 65.76 | 8.68 | 0.00 |

### Example 4. Provided Technologies can Effectively Induce Skipping of Exon 13 in USH2A Gene Transcripts

In some embodiments, a gel-shift assay and/or Sanger sequencing were utilized to assess the exon skipping.

In one example, Y-79 cell lines were treated with 20 uM WV-20902; RNA isolated 48 hours later. PCR amplified the exon 8-17 region using USH2A exon-specific primers (Exon8 to Exon17), samples were run on a gel. WV-20902 treated sample was lower on the gel and therefore has less base pairs comparing to the PBS treated samples. The difference observed was approximately 650 bp; exon 13 is 642 bp.

Sanger sequencing further confirmed the skipping by showing the difference of the junction. In PBS-treated sample, a junction of non-skipped exons was observed ( . . . GTTATTGGGCTTAGG . . . ) (SEQ ID NO: 341); in WV-20902 treated sample, a junction of exon skipping was observed ( . . . GTTATTGGTTTTTAT . . . ) (SEQ ID NO: 342).

### Example 5. Provided Technologies can Effectively Induce Exon Skipping In Vivo

Humanized USH2a exon 13 mouse lines were generated to study USH2A oligonucleotides.

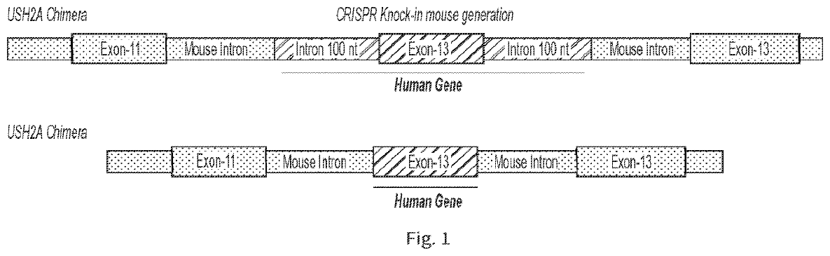

Two humanized USH2A exon 13 mouse lines were generated as shown in FIG. 1. Each line resulted from the replacement of mouse exon 12 with the homologous human exon 13. One animal line (top panel of FIG. 1) contained human exon 13 plus one hundred nucleotides of human intron 5′ and 3′ to human exon 13. The second line (bottom panel of FIG. 1) contained only human exon 13 with no inclusion of human intron. Human exon 13 is homologous to mouse exon 12.

For in vivo experiments described herein, the intron containing mouse line was used. C57BL/6 mice, approximately 8 weeks of age at time of dosing, were anesthetized with ketamine 30-40 mg/kg and xylazine 0.5-10 mg/kg. While anesthetized, a 3 uL drop of 0.5% proparacaine hydrochloride was applied to both eyes. A 100 uL nanofil syringe with a 33 g needle was inserted into the posterior chamber, 3 mm posterior to the limbus, taking care not to touch the iris or lens. The test article (1 uL) was injected into the posterior chamber of the eye using a micromanipulator and microinjection pump. Following injection, the needle was left in place for approximately 30 seconds. Test article was injected into each eye of all animals. Antibiotic ointment was applied to the eyes after injection. Once the procedure was complete, animals were monitored until recovered.

At the time of necropsy, eyes were enucleated and immediately frozen on dry ice. Each globe was bisected along the coronal plane to separate the anterior (cornea, iris, lens, posterior chamber, partial sclera) and the posterior (retina, choroid, sclera) portions of each eye.

For RNA isolation, frozen tissue was added to 700 uL of Triazol and homogenized for 3 minutes. Bromochloropropane was added to each sample, shaken vigorously, and centrifuged at 4000×g for 5 minutes. Supernatant (250 ul) was transferred to the binding plate from SV96 total RNA extraction kit (Promega) and RNA was extracted per protocol. cDNA was synthesized by adding 3 uL of total RNA to a 20 uL RT reaction using High Capacity cDNA Reverse Transcription Kit as recommended by the vendor (Thermo Fisher #4368814).

qPCR was performed by diluting 20 uL of cDNA with 30 uL of water. Four microliters of this solution was combined with USH2a primers (IDT) to detect skipped product and primers to detect unskipped product as well as qPCR master mix.

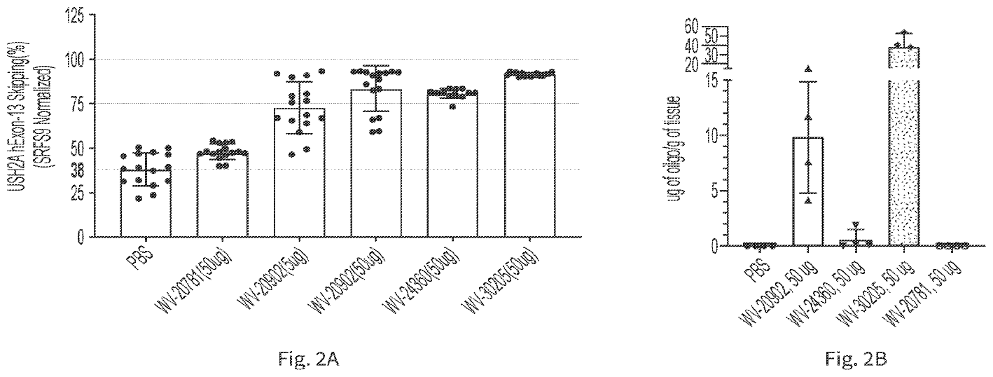

Animals treated with PBS were found to have some exon skipping in the posterior of the eye (retina, choroid, sclera combined), e.g., as illustrated in FIG. 2A. Such exon skipping was confirmed by RNA-Seq analysis and, without any intention to be bound by theory, is believed to be a product of the knock in procedure to generate the mice.

### Provided Technologies Provide High Efficiency of Exon Skipping.

Chirally controlled oligonucleotides compositions were found to be active (up to 90% skipping), more potent (5 ug of WV-20902 (chirally controlled) vs. 50 ug of WV-20781 (stereorandom)), and more efficacious (50 ug of 20902, 24360 and 30205 (chirally controlled) vs. 50 ug of 20781 (stereorandom)) than the stereorandom reference composition one week post a single intravitreal injection. Certain tissue exposure measurements in the posterior of the eye were presented in FIG. 2B (as shown, WV-30205>WV-20902>WV-24360; WV-20781 sample analysis not working in FIG. 2B).

As demonstrated, provided chirally controlled oligonucleotide composition of WV-30205 can provide dramatically higher exon skipping compared to stereorandom oligonucleotide composition of WV-20781 and PBS. One example set of RNA-seq analysis of posterior of the eye RNA from stereorandom oligonucleotide composition (WV-20781) and stereopure oligonucleotide composition (WV-30205) confirmed that newly formed skipped transcript levels in animals treated with the stereorandom oligonucleotide composition were no different than PBS-treated animals, and treatment with chirally controlled oligonucleotide compositions resulted in 3-fold higher newly skipped transcript levels than either treatment with PBS or stereorandom composition.

To generate a near complete protein (minus the skipped exon region), transcription/translation through exon 72 would be required. It was shown that all 72 exons except exon 12 (corresponding to human exon 13) were present following treatment with chirally controlled WV-30205 composition, and the only significant difference in transcripts counts was at exon 12 (human exon 13) with less exon 12 (human exon 13) transcripts present following treatment with chirally controlled WV-30205 composition (both at 75 and 150 ug dose levels). Treatment with stereorandom WV-20781 composition showed all 72 exons present after treatment but no significant difference in transcript counts for any exon relative to PBS treatment.

### Example 6. Provided Technologies can Effectively Induce Exon Skipping In Vivo

In some embodiments, provided technologies were assessed in non-human primate models. Among other things, it was demonstrated that provided technologies can effectively induce exon skipping.

In some embodiments, in an animal model USH2A exon 12 is homologous to human USH2A exon 13, and skipping of such exon 12 is assessed.

In some embodiments, non-naïve cynomolgus macaques -2.5-5 kg at time of dosing were anesthetized with Ketamine 5-15 mg/kg, IM. While anesthetized, 2-5 drops of 0.5% proparacaine was applied to both eyes to anesthetize the eye. After approximately 2 minutes, 1-2 drops of betadine (5%) was added to each eye and left for approximately 5 minutes. After 5 minutes, excess was wicked away with an ocular absorbent spear and rinsed with saline. The eye was held open with a speculum and positioned into place with a cotton tipped applicator. A 28-30 G insulin syringe (U-100) was used to inject (50 μL) into the eye at a 45 degree angle pointed towards the optic nerve (being careful not to hit the lens). Following the injection, the needle was slowly removed and the eye monitored for efflux. Antibiotic ointment was applied immediately post injection and animals were monitored until recovered.

At the time of necropsy, eyes were enucleated and dissected to isolate retina, choroid/sclera, cornea, iris and vitreous tissues. Each tissue was placed in a pre-labeled, pre-weighed 1.5 mL Eppendorf tube and stored at −80° C. until processed.

For RNA isolation, frozen tissue was added to 700 ul of Triazol and homoogenized for 3 minutes. Bromochloropropane was added to each sample, shaken vigorously, and centrifuged at 4000×g for 5 minutes. Supernatant (250 ul) was transferred to the binding plate from SV96 total RNA extraction kit (Promega) and RNA was extracted per protocol. cDNA was synthesized by adding 3 μL of total RNA to a 20 uL RT reaction using High Capacity cDNA Reverse Transcription Kit as recommended by the vendor (Thermo Fisher #4368814).

qPCR was performed by diluting 20 uL of cDNA with 30 uL of water. Four microliters of this solution was combined with USH2a primers (IDT) to detect skipped product and primers to detect unskipped product as well as qPCR master mix.

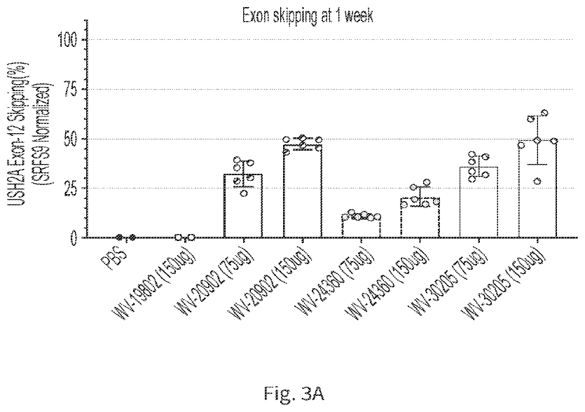

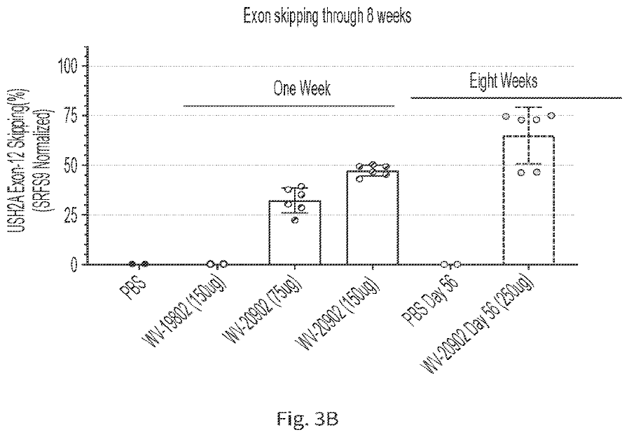

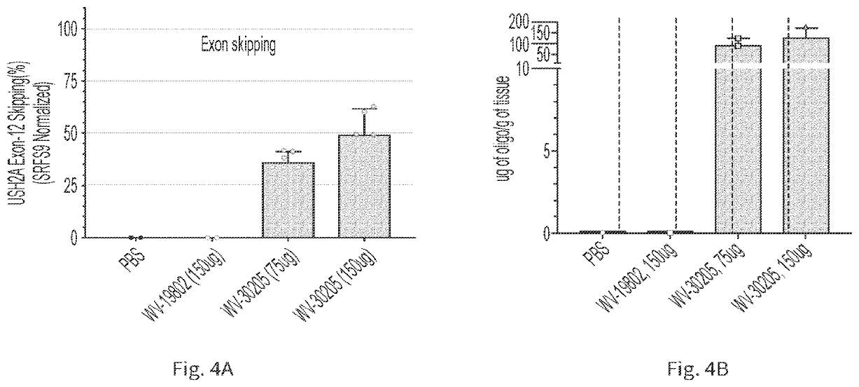

Dose dependent exon skipping up to 50% was observed in the NHP retina one week after a single intravitreal injection with WV-20902, 24360 and 30205 (FIG. 3A). Skipping was evaluated eight weeks following a single intravitreal injection of WV-20902 and resulted in ˜65% skipping (FIG. 3B). Certain exon skipping data were presented in FIG. 4A and certain tissue exposure data were presented in FIG. 4B, which showed dose dependence for WV-30205. As was found in certain mouse models, RNA-seq analysis confirmed that treatment with WV-30205 resulted in a significant reduction of exon 12 (NHP exon 12 is homologous to human exon 13) transcript counts relative to PBS and all 72 exons were transcribed for various dose treatment groups (75 ug and 150 ug).

In some embodiments, the present disclosure provides the Embodiments below as examples:

### EXAMPLE EMBODIMENTS

1. An oligonucleotide, wherein the base sequence of the oligonucleotide comprises at least 15, 16, 17, 18, 19, 20, 21, 22, 23, 24, or 25 contiguous nucleobases of a base sequence that is identical to or complementary to a base sequence of an USH2A gene or a transcript thereof, wherein the oligonucleotide comprises at least one chiral internucleotidic linkage comprising a stereodefined linkage phosphorus.

2. An oligonucleotide, wherein the base sequence of the oligonucleotide comprises at least 15, 16, 17, 18, 19, 20, 21, 22, 23, 24, or 25 contiguous nucleobases of a base sequence that is at least 75% identical or complementary to a base sequence of an USH2A gene or a transcript thereof, wherein the oligonucleotide comprises at least one chiral internucleotidic linkage comprising a stereodefined linkage phosphorus.

3. An oligonucleotide, wherein the base sequence of the oligonucleotide comprises at least 15, 16, 17, 18, 19, 20, 21, 22, 23, 24, or 25 contiguous nucleobases of a base sequence that is identical to or complementary to a base sequence of an USH2A gene or a transcript thereof, wherein the oligonucleotide comprises at least one chiral internucleotidic linkage comprising a stereodefined linkage phosphorus, and wherein the oligonucleotide is capable of increasing the level of skipping of a deleterious exon in an USH2A gene transcript or a gene product thereof.

4. An oligonucleotide, wherein the base sequence of the oligonucleotide comprises at least 15, 16, 17, 18, 19, 20, 21, 22, 23, 24, or 25 contiguous nucleobases of a base sequence that is at least 75% identical or complementary to a base sequence of an USH2A gene or a transcript thereof, wherein the oligonucleotide comprises at least one chiral internucleotidic linkage comprising a stereodefined linkage phosphorus, and wherein the oligonucleotide is capable of increasing the level of skipping of a deleterious exon in an USH2A gene transcript or a gene product thereof.

5. An USH2A oligonucleotide capable of mediating skipping of USH2A exon 13 has a sequence which hybridizes to (e.g., is complementary to a sequence of) an USH2A gene transcript sequence within exon 13, a sequence within an intron immediately adjacent to exon 13 (e.g., intron 12 or intron 13), or a sequence spanning the boundary between USH2A exon 13 and an intron immediately adjacent to exon 13 (e.g., intron 12 or intron 13).

6. An oligonucleotide, wherein the base sequence of the oligonucleotide comprises at least 15, 16, 17, 18, 19, 20, 21, 22, 23, 24, or 25 contiguous nucleobases of a base sequence that is identical to or complementary to a base sequence of an USH2A gene or a transcript thereof, wherein the oligonucleotide comprises at least one chiral internucleotidic linkage comprising a stereodefined linkage phosphorus, and wherein the oligonucleotide is capable of mediating the skipping of a deleterious exon in an USH2A gene transcript or a gene product thereof.

7. An oligonucleotide, whose base sequence is, comprises, or comprises a span of 10, 11, 12, 13, 14, 15, 16, 17, 18, 19 or 20 contiguous nucleobases of any base sequences in Table A1.

8. The oligonucleotide of any one of the preceding Embodiments, wherein the oligonucleotide comprises at least two chiral internucleotidic linkages comprising a stereodefined linkage phosphorus.

9. The oligonucleotide of any one of the preceding Embodiments, wherein the oligonucleotide comprises at least 5, 6, 7, 8, 9, or 10 chiral internucleotidic linkages comprising a stereodefined linkage phosphorus.

10. The oligonucleotide of any one of the preceding Embodiments, wherein the oligonucleotide is capable of increasing the level of skipping of a deleterious exon in an USH2A gene transcript or a gene product thereof.

11. The oligonucleotide of any one of the preceding Embodiments, wherein the USH2A target gene is a mutant USH2A target gene associated with an USH2A-related condition, disorder or disease.

12. The oligonucleotide of any one of the preceding Embodiments, wherein the USH2A target gene is a wild-type USH2A target gene.

13. The oligonucleotide of any one of the preceding Embodiments, wherein one or more internucleotidic linkages each independently comprise a stereodefined linkage phosphorus in the Rp configuration.

14. The oligonucleotide of any one of the preceding Embodiments, wherein one or more internucleotidic linkages each independently comprise a stereodefined linkage phosphorus in the Sp configuration.

15. The oligonucleotide of any one of the preceding Embodiments, wherein at least 60%, 70%, 75%, 80%, 85%, 90% or 95% of all chirally controlled phosphorothioate internucleotidic linkages are Sp.

16. The oligonucleotide of any one of the preceding Embodiments, wherein at least 60%, 70%, 75%, 80%, 85%, 90% or 95% of all chirally controlled non-negatively charged internucleotidic linkages are Rp.

17. The oligonucleotide of any one of the preceding Embodiments, wherein at least 60%, 70%, 75%, 80%, 85%, 90% or 95% of all chirally controlled internucleotidic linkages are Sp.

18. The oligonucleotide of any one of the preceding Embodiments, wherein at least 60%, 70%, 75%, 80%, 85%, 90% or 95% of all modified internucleotidic linkages are phosphorothioate internucleotidic linkages.

19. The oligonucleotide of any one of the preceding Embodiments, wherein at least 60%, 70%, 75%, 80%, 85%, 90% or 95% of all modified internucleotidic linkages are phosphorothioate internucleotidic linkages having a Sp configuration.

20. The oligonucleotide of any one of the preceding Embodiments, wherein at least 60%, 70%, 75%, 80%, 85%, 90% or 95% of all internucleotidic linkages are phosphorothioate internucleotidic linkages.

21. The oligonucleotide of any one of the preceding Embodiments, wherein at least 60%, 70%, 75%, 80%, 85%, 90% or 95% of all internucleotidic linkages are phosphorothioate internucleotidic linkages having a Sp configuration.

22. An oligonucleotide, wherein the oligonucleotide comprises a plurality of chiral internucleotidic linkages each of which independently comprises a stereodefined linkage phosphorus, wherein the pattern of backbone chiral centers of the oligonucleotide comprises [(Rp/Op)n(Sp)m]y, wherein:
n is 1-10;
m is 1-50;
y is 2-10;
Op indicates a linkage phosphorus being achiral;
Rp indicates a linkage phosphorus having R configuration;
Sp indicates a linkage phosphorus having S configuration; and
at least one [(Rp/Op)n(Sp)m] comprises RpSpSp.

23. The oligonucleotide of Embodiment 22, wherein the base sequence of the oligonucleotide is or comprises a complementary sequence that is complementary to a target sequence in an USH2A gene or a transcript thereof.

24. The oligonucleotide of any one of Embodiments 22-23, wherein the target base sequence in an USH2A gene or a transcript thereof is a characteristic sequence of the USH2A gene or a transcript thereof.

25. The oligonucleotide of any one of Embodiments 22-24, wherein the oligonucleotide is capable of increasing the level of skipping of a deleterious exon in an USH2A gene transcript or a gene product thereof.

26. The oligonucleotide of any one of Embodiments 22-25, wherein the USH2A target gene is a mutant USH2A target gene associated with an USH2A-related condition, disorder or disease.

27. The oligonucleotide of any one of Embodiments 22-25, wherein the USH2A target gene is a wild-type USH2A target gene.

28. The oligonucleotide of any one of Embodiments 22-27, wherein the base sequence of the oligonucleotide comprises 10, 11, 12, 13, 14, 15, 16, 17, 18, 19, 20, 21, 22, 23, 24, or 25 or more contiguous nucleobases of a base sequence that is identical to or complementary to a base sequence of an USH2A gene or a transcript thereof.

29. The oligonucleotide of any one of the preceding Embodiments, wherein a transcript is an USH2A mRNA.

30. The oligonucleotide of any one of the preceding Embodiments, wherein the sequence of the contiguous nucleobases is characteristic of the USH2A gene or a transcript thereof in that it is not identical or complementary to any other non-USH2A sequences or transcripts thereof.

31. The oligonucleotide of any one of the preceding Embodiments, wherein the oligonucleotide is capable of selectively increasing the level of skipping of a deleterious exon in an USH2A gene transcript that is associated with a condition, disorder or disease.

32. The oligonucleotide of Embodiment 31, wherein the condition, disorder or disease is Usher Syndrome.

33. The oligonucleotide of Embodiment 31, wherein the condition, disorder or disease is Usher Syndrome type 2A.

34. The oligonucleotide of any one of Embodiment 31-33, wherein the USH2A gene associated with Usher Syndrome comprises at least one disease-associated mutation.

35. The oligonucleotide of any one of the preceding Embodiments, wherein the oligonucleotide is capable of selectively increasing the level of skipping of human exon 13 over skipping of human exon 12 or skipping of human exons 12 and 13.

36. The oligonucleotide of any one of Embodiments 31-35, wherein the selectivity is at least 2, 3, 4, 5, 6, 7, 8, 9, 10, 15, 20, 25, 30, 35, 40, 50, 60, 70, 80, 90 or 100 fold.

37. The oligonucleotide of any one of Embodiments 31-36, wherein the selectivity is at least 2 fold.

38. The oligonucleotide of any one of the preceding Embodiments, wherein the oligonucleotide has the structure of formula O-I or a salt thereof.

39. The oligonucleotide of any one of the preceding Embodiments, wherein the base sequence of the oligonucleotide is, comprises, or comprises a span of 10, 11, 12, 13, 14, 15, 16, 17, 18, 19 or 20 contiguous nucleobases of UGCAGAAUUUGUUCACUGAG (SEQ ID NO: 25), AAGCCCUAAAGAUAAAAUAU (SEQ ID NO: 1), AAUACAUUUCUUUCUUACCU (SEQ ID NO: 2), ACAUCCAACAUCAUUAAAGC (SEQ ID NO: 3), AGCUUCGGAGAAAUUUAAAUC (SEQ ID NO: 4), AGCUUCGGAGAAAUUUAAAUC (SEQ ID NO: 5), AGGAUUGCAGAAUUUGUUCA (SEQ ID NO: 6), AGGAUUGCAGAAUUUGUUCA (SEQ ID NO: 7), AUCCAAAAUUGCAAUGAUCA (SEQ ID NO: 8), AUUUCUUUCUUACCUGGUUG (SEQ ID NO: 9), CAACAUCAUUAAAGCUUCGG (SEQ ID NO: 10), CACCUAAGCCCUAAAGAUAA (SEQ ID NO: 11), GAGGAUUGCAGAAUUUGUUC (SEQ ID NO: 12), GAUCACACCUAAGCCCUAAA (SEQ ID NO: 13), GAUUGCAGAAUUUGUUCACU (SEQ ID NO: 14), GCAAUGAUCACACCUAAGCC (SEQ ID NO: 15), GCUUCGGAGAAAUUUAAAUC (SEQ ID NO: 16), GGAAUCACACUCACACAUCU (SEQ ID NO: 17), GGAUUGCAGAAUUUGUUCAC (SEQ ID NO: 18), GGAUUGCAGAAUUUGUUCAC (SEQ ID NO: 19), UACCUGGUUGACACUGAUUA (SEQ ID NO: 20), UACCUGGUUGACACUGAUUA (SEQ ID NO: 21), UCUUUUUUGCACUCACACUG (SEQ ID NO: 22), UGAGGAUUGCAGAAUUUGUU (SEQ ID NO: 23), UGAGGAUUGCAGAAUUUGUU (SEQ ID NO: 24), UUGCAGAAUUUGUUCACUGA (SEQ ID NO: 26), or UUUCUUACCUGGUUGACACU (SEQ ID NO: 27), wherein each U can be independently replaced by T and each U can be independently replaced with T.

40. The oligonucleotide of any one of the preceding Embodiments, wherein the base sequence of the oligonucleotide is, comprises, or comprises a span of 10, 11, 12, 13, 14, or 15 contiguous nucleobases of UGCAGAAUUUGUUCACUGAG (SEQ ID NO: 25), wherein each U can be independently replaced by T.

41. The oligonucleotide of any one of the preceding Embodiments, wherein the base sequence of the oligonucleotide is, comprises, or comprises a span of 10, 11, 12, 13, 14, or 15 contiguous nucleobases of AAGCCCUAAAGAUAAAAUAU (SEQ ID NO: 1), wherein each U can be independently replaced by T.

42. The oligonucleotide of any one of the preceding Embodiments, wherein the base sequence of the oligonucleotide is, comprises, or comprises a span of 10, 11, 12, 13, 14, 15, or 16 contiguous nucleobases of AAUACAUUUCUUUCUUACCU (SEQ ID NO: 2), wherein each U can be independently replaced by T.

43. The oligonucleotide of any one of the preceding Embodiments, wherein the base sequence of the oligonucleotide is, comprises, or comprises a span of 10, 11, 12, 13, 14, 15, 16, 17, 18, 19, 20 or 21 contiguous nucleobases of AGCUUCGGAGAAAUUUAAAUC (SEQ ID NO: 4), wherein each U can be independently replaced by T.

44. The oligonucleotide of any one of the preceding Embodiments, wherein the base sequence of the oligonucleotide is, comprises, or comprises a span of 10, 11, 12, 13, 14, 15, 16, 17, 18, 19, or 20 contiguous nucleobases of AGCUUCGGAGAAAUUUAAAUC (SEQ ID NO: 5), wherein each U can be independently replaced by T or vice versa.

45. The oligonucleotide of any one of the preceding Embodiments, wherein the base sequence of the oligonucleotide is, comprises, or comprises a span of 10, 11, 12, 13, 14, 15, 16, 17, 18, 19, 20 or 21 contiguous nucleobases of AGGAUUGCAGAAUUUGUUCA (SEQ ID NO: 6), wherein each U can be independently replaced by T or vice versa.

46. The oligonucleotide of any one of the preceding Embodiments, wherein the base sequence of the oligonucleotide is, comprises, or comprises a span of 10, 11, 12, 13, 14, 15, 16, 17, 18, 19, or 20 contiguous nucleobases of AGGAUUGCAGAAUUUGUUCA (SEQ ID NO: 7), wherein each U can be independently replaced by T or vice versa.

47. The oligonucleotide of any one of the preceding Embodiments, wherein the base sequence of the oligonucleotide is, comprises, or comprises a span of 10, 11, 12, 13, 14, 15, 16, 17, 18, 19 or 20 contiguous nucleobases of AUCCAAAAUUGCAAUGAUCA (SEQ ID NO: 8), wherein each U can be independently replaced by T or vice versa.

48. The oligonucleotide of any one of the preceding Embodiments, wherein the base sequence of the oligonucleotide is, comprises, or comprises a span of 10, 11, 12, 13, 14, 15, 16, 17, 18, 19 or 20 contiguous nucleobases of AUUUCUUUCUUACCUGGUUG (SEQ ID NO: 9), wherein each U can be independently replaced by T or vice versa.

49. The oligonucleotide of any one of the preceding Embodiments, wherein the base sequence of the oligonucleotide is, comprises, or comprises a span of 10, 11, 12, 13, 14, 15, 16, 17, 18, 19 or 20 contiguous nucleobases of CAACAUCAUUAAAGCUUCGG (SEQ ID NO: 10), wherein each U can be independently replaced by T or vice versa.
wherein each T can be independently and optionally replaced by U.

50. The oligonucleotide of any one of the preceding Embodiments, wherein the base sequence of the oligonucleotide is, comprises, or comprises a span of 10, 11, 12, 13, 14, 15, 16, 17, 18, 19 or 20 contiguous nucleobases of CACCUAAGCCCUAAAGAUAA (SEQ ID NO: 11), wherein each U can be independently replaced by T or vice versa.

51. The oligonucleotide of any one of the preceding Embodiments, wherein the base sequence of the oligonucleotide is, comprises, or comprises a span of 10, 11, 12, 13, 14, 15, 16, 17, 18, 19 or 20 contiguous nucleobases of GAGGAUUGCAGAAUUUGUUC (SEQ ID NO: 12), wherein each U can be independently replaced by T or vice versa.

52. The oligonucleotide of any one of the preceding Embodiments, wherein the base sequence of the oligonucleotide is, comprises, or comprises a span of 10, 11, 12, 13, 14, 15, 16, 17, 18, 19 or 20 contiguous nucleobases of GAUCACACCUAAGCCCUAAA (SEQ ID NO: 13), wherein each U can be independently replaced by T or vice versa.

53. The oligonucleotide of any one of the preceding Embodiments, wherein the base sequence of the oligonucleotide is, comprises, or comprises a span of 10, 11, 12, 13, 14, 15, 16, 17, 18, 19 or 20 contiguous nucleobases of GAUUGCAGAAUUUGUUCACU (SEQ ID NO: 14), UUUCUUACCUGGUUGACACU (SEQ ID NO: 27), wherein each U can be independently replaced by T or vice versa.

54. The oligonucleotide of any one of the preceding Embodiments, wherein the base sequence of the oligonucleotide is, comprises, or comprises a span of 10, 11, 12, 13, 14, 15, 16, 17, 18, 19 or 20 contiguous nucleobases of GCAAUGAUCACACCUAAGCC (SEQ ID NO: 15), wherein each U can be independently replaced by T or vice versa.

55. The oligonucleotide of any one of the preceding Embodiments, wherein the base sequence of the oligonucleotide is, comprises, or comprises a span of 10, 11, 12, 13, 14, 15, 16, 17, 18, 19 or 20 contiguous nucleobases of GCUUCGGAGAAAUUUAAAUC (SEQ ID NO: 16), wherein each U can be independently replaced by T or vice versa.

56. The oligonucleotide of any one of the preceding Embodiments, wherein the base sequence of the oligonucleotide is, comprises, or comprises a span of 10, 11, 12, 13, 14, 15, 16, 17, 18, 19 or 20 contiguous nucleobases of GGAAUCACACUCACACAUCU (SEQ ID NO: 17), wherein each U can be independently replaced by T or vice versa.

57. The oligonucleotide of any one of the preceding Embodiments, wherein the base sequence of the oligonucleotide is, comprises, or comprises a span of 10, 11, 12, 13, 14, 15, 16, 17, 18, 19 or 20 contiguous nucleobases of GGAUUGCAGAAUUUGUUCAC (SEQ ID NO: 18), wherein each U can be independently replaced by T or vice versa.

58. The oligonucleotide of any one of the preceding Embodiments, wherein the base sequence of the oligonucleotide is, comprises, or comprises a span of 10, 11, 12, 13, 14, 15, 16, 17, 18, 19 or 20 contiguous nucleobases of GGAUUGCAGAAUUUGUUCAC (SEQ ID NO: 19), wherein each U can be independently replaced by T or vice versa.

59. The oligonucleotide of any one of the preceding Embodiments, wherein the base sequence of the oligonucleotide is, comprises, or comprises a span of 10, 11, 12, 13, 14, 15, 16, 17, 18, 19 or 20 contiguous nucleobases of UACCUGGUUGACACUGAUUA (SEQ ID NO: 20), UACCUGGUUGACACUGAUUA (SEQ ID NO: 21), wherein each U can be independently replaced by T or vice versa.

60. The oligonucleotide of any one of the preceding Embodiments, wherein the base sequence of the oligonucleotide is, comprises, or comprises a span of 10, 11, 12, 13, 14, 15, 16, 17, 18, 19 or 20 contiguous nucleobases of UACCUGGUUGACACUGAUUA (SEQ ID NO: 21), UCUUUUUUGCACUCACACUG (SEQ ID NO: 22), UGAGGAUUGCAGAAUUUGUU (SEQ ID NO: 23), UGAGGAUUGCAGAAUUUGUU (SEQ ID NO: 24), UGCAGAAUUUGUUCACUGAG (SEQ ID NO: 25), UUGCAGAAUUUGUUCACUGA (SEQ ID NO: 26), or UUUCUUACCUGGUUGACACU (SEQ ID NO: 27), wherein each U can be independently replaced by T or vice versa.

61. The oligonucleotide of any one of any one of the preceding Embodiments, wherein at least one T is independently replaced by U.

62. The oligonucleotide of any one of Embodiments 1-60, wherein no T is replaced by U.

63. The oligonucleotide of any one of the preceding Embodiments, wherein the oligonucleotide comprises a plurality of chiral internucleotidic linkages each of which independently comprises a stereodefined linkage phosphorus, wherein the pattern of backbone chiral centers of the oligonucleotide comprises [(Rp/Op)n(Sp)m]y, wherein:
n is 1-10;
m is 1-50;
y is 1-10;
Op indicates a linkage phosphorus being achiral;
Rp indicates a linkage phosphorus having R configuration;
Sp indicates a linkage phosphorus having S configuration; and
at least one [(Rp/Op)n(Sp)m] comprises OpSpSp.

64. The oligonucleotide of any one of the preceding Embodiments, wherein the pattern of backbone chiral centers comprises (Sp)t[(Rp/Op)n(Sp)m]y, wherein t is 1-50.

65. The oligonucleotide of any one of the preceding Embodiments, wherein the pattern of backbone chiral centers comprises (Sp)t[(Rp/Op)n(Sp)m]yRp, wherein t is 1-50.

66. The oligonucleotide of any one of the preceding Embodiments, wherein the pattern of backbone chiral centers comprises Rp(Sp)t[(Rp/Op)n(Sp)m]y, wherein t is 1-50.

67. The oligonucleotide of any one of the preceding Embodiments, wherein the pattern of backbone chiral centers comprises Rp(Sp)t[(Rp/Op)n(Sp)m]yRp, wherein t is 1-50.

68. The oligonucleotide of any one of the preceding Embodiments, wherein at least one [(Rp/Op)n(Sp)m] is independently [(Op)n(Sp)m].

69. The oligonucleotide of any one of the preceding Embodiments, wherein each [(Rp/Op)n(Sp)m] is independently [Rp(Sp)m].

70. The oligonucleotide of any one of Embodiments 1-69, wherein t is 2-50.

71. The oligonucleotide of any one of the preceding Embodiments, wherein each Op indicates a linkage phosphorus being achiral in a natural phosphate linkage.

72. The oligonucleotide of any one of the preceding Embodiments, wherein at least one chiral internucleotidic linkage comprising a stereodefined linkage phosphorus is a phosphorothioate internucleotidic linkage.

73. The oligonucleotide of any one of the preceding Embodiments, wherein the oligonucleotide comprises a modified sugar.

74. The oligonucleotide of any one of the preceding Embodiments, wherein each sugar independently has the structure of

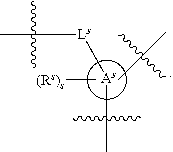

75. The oligonucleotide of any one of the preceding Embodiments, wherein each

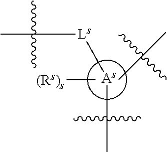

is independently

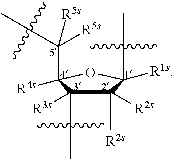

76. The oligonucleotide of Embodiment 75, wherein an occurrence of R^{5s} is —H.

77. The oligonucleotide of any one of Embodiments 75-76, wherein an occurrence of R^{4s} is —H.

78. The oligonucleotide of any one of Embodiments 75-76, wherein each occurrence of R^{4s} is independently —H, or is taken together with a R^{2s} to form.

79. The oligonucleotide of any one of Embodiments 75-78, wherein an occurrence of R^{3s} is —H.

80. The oligonucleotide of any one of Embodiments 75-78, wherein each occurrence of R^{3s} is —H.

81. The oligonucleotide of any one of Embodiments 75-80, wherein an occurrence of R^{2s} is —H.

82. The oligonucleotide of any one of Embodiments 75-81, wherein an occurrence of R^{1s} is —H.

83. The oligonucleotide of any one of Embodiments 75-81, wherein each occurrence of R^{1s} is —H.

84. The oligonucleotide of Embodiment 75, wherein each

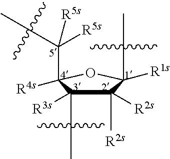

is independently

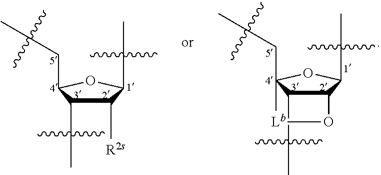

85. The oligonucleotide of any one of Embodiments 75-84, wherein an occurrence of R^{2s} is —H.

86. The oligonucleotide of any one of Embodiments 75-85, wherein an occurrence of R^{2s} is —F.

87. The oligonucleotide of any one of Embodiments 75-86, wherein an occurrence of R^{2s} is —OR, wherein R is optionally substituted C₁₋₆ alkyl.

88. The oligonucleotide of any one of Embodiments 75-87, wherein an occurrence of R^{2s} is —OMe.

89. The oligonucleotide of any one of Embodiments 75-88, wherein an occurrence of R^{2s} is —OCH₂CH₂OCH₃.

90. The oligonucleotide of any one of Embodiments 75-89, wherein an occurrence of R^{2s} is taken together with R^{4s}—OCH₂CH₂OCH₃.

91. The oligonucleotide of any one of Embodiments 75-90, wherein an occurrence of L^{b} is optionally substituted —CH₂—.

92. The oligonucleotide of any one of Embodiments 75-91, wherein each occurrence of L^{b} is independently optionally substituted —CH₂—.

93. The oligonucleotide of any one of Embodiments 75-92, wherein an occurrence of L^{b} is —CH₂—.

94. The oligonucleotide of any one of Embodiments 75-93, wherein each occurrence of L^{b} is —CH₂—.

95. The oligonucleotide of any one of the preceding Embodiments, wherein the oligonucleotide comprises 10, 11, 12, 13, 14, 15, 16, 17, 18, 19, 20 or more nucleobases.

96. The oligonucleotide of Embodiment 79, wherein each nucleobase independently comprises an optionally substituted aromatic ring.

97. The oligonucleotide of Embodiment 79, wherein each nucleobase independently optionally substituted A, T, C, G, or U, or an optionally substituted tautomer of A, T, C, G, or U.

98. The oligonucleotide of any one of the preceding Embodiments, wherein the oligonucleotide comprises 1-50, or 1, 2, 3, 4, 5, 6, 7, 8, 9, 10, 11, 12, 13, 14, 15, 16, 17, 18, 19, 20, 21, 22, 23, 24, 25, or 26 nucleobases.

99. The oligonucleotide of any one of the preceding Embodiments, wherein the oligonucleotide comprises 1, 2, 3, 4, 5, 6, 7, 8, 9, 10, 11, 12, 13, 14, 15, 16, 17, 18, 19, 20, 21, 22, 23, 24, 25, or 26 nucleobases.

100. The oligonucleotide of any one of the preceding Embodiments, wherein the oligonucleotide comprises 13 to 26 nucleobases.

101. The oligonucleotide of any one of the preceding Embodiments, wherein the oligonucleotide comprises 19, 20 or 21 nucleobases.

102. The oligonucleotide of any one of the preceding Embodiments, wherein the oligonucleotide comprises 19 nucleobases.

103. The oligonucleotide of any one of the preceding Embodiments, wherein the oligonucleotide comprises 20 nucleobases.

104. The oligonucleotide of any one of the preceding Embodiments, wherein the oligonucleotide consists of or comprises a structure of 5′-a first region-a second region-a third region-3′, wherein each of the regions independently comprises 1-20 (e.g., 1, 2, 3, 4, 5, 6, 7, 8, 9, 10, 11, 12, 13, 14, 15, 16, 17, 18, 19, 20) or more nucleosides.

105. The oligonucleotide of any one of the preceding Embodiments, wherein the first region comprises 2, 3, 4, 5, 6, 7, 8, 9, 10, 11, 12, 13, 14, 15, 16, 17, 18, 19, or 20 or more nucleosides.

106. The oligonucleotide of any one of the preceding Embodiments, wherein the first region comprises 5 or more nucleosides.

107. The oligonucleotide of any one of the preceding Embodiments, wherein the first region comprises one or more (e.g., 1-20, 1, 2, 3, 4, 5, 6, 7, 8, 9, 10, 11, 12, 13, 14, 15, 16, 17, 18, 19, or 20) 2′-F modified sugars.

108. The oligonucleotide of any one of the preceding Embodiments, wherein the first region comprises two or more (e.g., 2-20, 2, 3, 4, 5, 6, 7, 8, 9, 10, 11, 12, 13, 14, 15, 16, 17, 18, 19, or 20) consecutive 2′-F modified sugars.

109. The oligonucleotide of any one of the preceding Embodiments, wherein all 2′-F modified sugars in a first region are consecutive.

110. The oligonucleotide of any one of the preceding Embodiments, wherein at least 10%, 20%, 30%, 40%, 50%, 60%, 70%, 75%, 80%, 85%, 90%, or 95%, or 100% of all sugars in a first region comprises 2′-F.

111. The oligonucleotide of any one of the preceding Embodiments, wherein at least 50% of all sugars in a first region comprises 2′-F.

112. The oligonucleotide of any one of the preceding Embodiments, wherein each sugar in a first region comprises 2′-F.

113. The oligonucleotide of any one of the preceding Embodiments, wherein the second region comprises 2, 3, 4, 5, 6, 7, 8, 9, 10, 11, 12, 13, 14, 15, 16, 17, 18, 19, or 20 or more nucleosides.

114. The oligonucleotide of any one of the preceding Embodiments, wherein the second region comprises 4 or more nucleosides.

115. The oligonucleotide of any one of the preceding Embodiments, wherein the second region comprises one or more (e.g., 1-20, 1, 2, 3, 4, 5, 6, 7, 8, 9, 10, 11, 12, 13, 14, 15, 16, 17, 18, 19, or 20) 2′-F modified sugars.

116. The oligonucleotide of any one of the preceding Embodiments, wherein the second region comprises two or more (e.g., 2-20, 2, 3, 4, 5, 6, 7, 8, 9, 10, 11, 12, 13, 14, 15, 16, 17, 18, 19, or 20) consecutive 2′-F modified sugars.

117. The oligonucleotide of any one of the preceding Embodiments, wherein at least 10%, 20%, 30%, 40%, 50%, 60%, 70%, 75%, 80%, 85%, 90%, or 95%, or 100% of all sugars in a second region comprises 2′-F.

118. The oligonucleotide of any one of the preceding Embodiments, wherein at least 50% of all sugars in a second region comprises 2′-F.

119. The oligonucleotide of any one of the preceding Embodiments, wherein the second region comprises one or more (e.g., 1-20, 1, 2, 3, 4, 5, 6, 7, 8, 9, 10, 11, 12, 13, 14, 15, 16, 17, 18, 19, or 20) 2′-OR modified sugars, wherein R is optionally substituted C₁₋₆ aliphatic.

120. The oligonucleotide of any one of the preceding Embodiments, wherein the second region comprises two or more (e.g., 2-20, 2, 3, 4, 5, 6, 7, 8, 9, 10, 11, 12, 13, 14, 15, 16, 17, 18, 19, or 20) consecutive 2′-OR modified sugars, wherein R is optionally substituted C₁₋₆ aliphatic.

121. The oligonucleotide of any one of the preceding Embodiments, wherein at least 10%, 20%, 30%, 40%, 50%, 60%, 70%, 75%, 80%, 85%, 90%, or 95%, or 100% of all sugars in a second region comprises 2′-OR modified sugars, wherein R is optionally substituted C₁₋₆ aliphatic.

122. The oligonucleotide of any one of the preceding Embodiments, wherein at least 50% of all sugars in a second region comprises 2′-OR modified sugars, wherein R is optionally substituted C₁₋₆ aliphatic.

123. The oligonucleotide of any one of the preceding Embodiments, wherein all 2′-F modified sugars in a second region are consecutive.

124. The oligonucleotide of any one of the preceding Embodiments, wherein all 2′-OR modified sugars in a second region are consecutive.

125. The oligonucleotide of any one of Embodiments 113-122, wherein the second region comprise alternating 2′-F and 2′-OR modifications.

126. The oligonucleotide of any one of Embodiments 113-125, wherein 2′-OR is 2′-OMe.

127. The oligonucleotide of any Embodiments 113-118, wherein each sugar in a second region comprises 2′-F.

128. The oligonucleotide of any one of the preceding Embodiments, wherein the third region comprises 2, 3, 4, 5, 6, 7, 8, 9, 10, 11, 12, 13, 14, 15, 16, 17, 18, 19, or 20 or more nucleosides.

129. The oligonucleotide of any one of the preceding Embodiments, wherein the third region comprises 5 or more nucleosides.

130. The oligonucleotide of any one of the preceding Embodiments, wherein the third region comprises one or more (e.g., 1-20, 1, 2, 3, 4, 5, 6, 7, 8, 9, 10, 11, 12, 13, 14, 15, 16, 17, 18, 19, or 20) 2′-F modified sugars.

131. The oligonucleotide of any one of the preceding Embodiments, wherein the third region comprises two or more (e.g., 2-20, 2, 3, 4, 5, 6, 7, 8, 9, 10, 11, 12, 13, 14, 15, 16, 17, 18, 19, or 20) consecutive 2′-F modified sugars.

132. The oligonucleotide of any one of the preceding Embodiments, wherein all 2′-F modified sugars in a third region are consecutive.

133. The oligonucleotide of any one of the preceding Embodiments, wherein at least 10%, 20%, 30%, 40%, 50%, 60%, 70%, 75%, 80%, 85%, 90%, or 95%, or 100% of all sugars in a third region comprises 2′-F.

134. The oligonucleotide of any one of the preceding Embodiments, wherein at least 50% of all sugars in a third region comprises 2′-F.

135. The oligonucleotide of any one of the preceding Embodiments, wherein each sugar in a third region comprises 2′-F.

136. The oligonucleotide of any one of Embodiments 95-103, wherein the oligonucleotide no sugar modification that is 2′-OR, wherein R is optionally substituted C₁₋₆ alkyl.

137. The oligonucleotide of any one of Embodiments 95-103, wherein the oligonucleotide comprises a sugar modification that is 2′-F.

138. The oligonucleotide of any one of Embodiments 95-103, wherein each sugar of the oligonucleotide is 2′-F or 2′-OR, wherein R is optionally substituted C₁₋₆ aliphatic.

139. The oligonucleotide of any one of Embodiments 95-103, wherein each sugar of the oligonucleotide is 2′-F or 2′-OMe.

140. The oligonucleotide of any one of Embodiments 95-103, wherein the oligonucleotide comprises at least one sugar modification that is 2′-F and at least one sugar modification that is 2′-OMe.

141. The oligonucleotide of any one of Embodiments 95-103, wherein the oligonucleotide comprises at least two sugar modifications that are 2′-F and at least one sugar modification that is 2′-OMe 142. The oligonucleotide of any one of Embodiments 95-141, wherein a sugar comprises a 2′-modification.

143. The oligonucleotide of Embodiment 142, wherein the 2′-modification is 2′-OR, wherein R is optionally substituted C₁₋₆ alkyl.

144. The oligonucleotide of Embodiment 143, wherein R is —CH₃.

145. The oligonucleotide of Embodiment 143, wherein R is —CH₂CH₂OCH₃.

146. The oligonucleotide of any of any one of the preceding Embodiments, wherein the pattern of stereochemistry and linkage of the oligonucleotide is or comprises: SSSSSSSSSSSSSSSSSSS, SSSSSSSSSS, SSSSSSSSOSSSOSSSSSS, SSSSSSSSOSSSOSSSSSS, SSSSSSOSSSSOOSSSSSS, SSSOSSSOSSSSS, SSSOSSSOSSSS, SSOSSSSOOSSSS, SSOSSSSOOS, SSOSSSOSSSS, SSOSSSOSSS, SSOSSSOSSS, SSnXSSSSSSSSSSnXSSS, SSnXSSOSSSOSS, SSnXSSOSSSOS, SSnXSSnXSSSSSSSSSSnXSSS, SSnXSSnXSSSSSSSSSSnXSS, SSnXSSnXSSSSSSSSSSnXSS, SSnXSSnXSSSSSSSSS, SSnXSSnXSSOSSSOSSSnXSS, SSnXSSnXSSOSSSOSSS, SOSSSSOOSSSSS, SOSSSSOOSS, SOSSSOSS, SOSSSOS, SnXSSSSSSSSSSnXSS, SnXSSOSSSOSSSnX, SnXSSOSSSOSSS, wherein O is a natural phosphate internucleotidic linkage, S is a phosphorothioate in the Sp configuration, and nX is a non-negatively charged internucleotidic linkage.

147. The oligonucleotide of any one of the preceding Embodiments, wherein the pattern of stereochemistry and linkage of the oligonucleotide is or comprises: SSnXSSOSSSOS, SSnXSSnXSSSSSSSSSSnXSSS, SSnXSSnXSSSSSSSSSSnXSS, SSnXSSnXSSSSSSSSSSnXSS, SSnXSSnXSSSSSSSSS, SSnXSSnXSSOSSSOSSSnXSS, SSnXSSnXSSOSSSOSSS, SOSSSSOOSSSSS, wherein O is a natural phosphate internucleotidic linkage, S is a phosphorothioate in the Sp configuration, and nX is a non-negatively charged internucleotidic linkage.

148. The oligonucleotide of any one of the preceding Embodiments, wherein the pattern of stereochemistry and linkage of the oligonucleotide is or comprises: SSSSSSSSSS, SSSSSSSSOSSSOSSSSSS, SSOSSSSOOSSSS, SSOSSSSOOS, SSOSSSOSSSS, SSOSSSOSSS, SSOSSSOSSS, SSnXSSSSSSSSSSnXSSS, SSnXSSOSSSOSS, wherein O is a natural phosphate internucleotidic linkage, S is a phosphorothioate in the Sp configuration, and nX is a non-negatively charged internucleotidic linkage.

149. The oligonucleotide of any one of the preceding Embodiments, wherein the pattern of stereochemistry and linkage of the oligonucleotide is or comprises: SSSSSSSSSS, SSSSSSSSOSSSOSSSSSS, SSnXSSSSSSSSSSnXSSS, SSnXSSOSSSOSS, SSnXSSOSSSOS, SSnXSSnXSSSSSSSSSSnXSSS, SSnXSSnXSSSSSSSSSSnXSS, SSnXSSnXSSSSSSSSSSnXSS, SSnXSSnXSSSSSSSSS, wherein O is a natural phosphate internucleotidic linkage, S is a phosphorothioate in the Sp configuration, and nX is a non-negatively charged internucleotidic linkage.

150. The oligonucleotide of any one of the preceding Embodiments, wherein the pattern of stereochemistry and linkage of the oligonucleotide is or comprises: SSSSSSSSSS, SSSOSSSOSSSS, SSOSSSSOOSSSS, SSOSSSSOOS, SSOSSSOSSSS, SSOSSSOSSS, SSOSSSOSSS, SSnXSSSSSSSSSSnXSSS, SSnXSSOSSSOSS, SSnXSSOSSSOS, wherein O is a natural phosphate internucleotidic linkage, S is a phosphorothioate in the Sp configuration, and nX is a non-negatively charged internucleotidic linkage.

151. The oligonucleotide of any one of the preceding Embodiments, wherein the pattern of stereochemistry and linkage of the oligonucleotide is or comprises: SSSSSSOSSSSOOSSSSSS, SSSOSSSOSSSSS, SSSOSSSOSSSS, SSOSSSSOOSSSS, SSOSSSSOOS, SSOSSSOSSSS, SSOSSSOSSS, SSOSSSOSSS, wherein O is a natural phosphate internucleotidic linkage, S is a phosphorothioate in the Sp configuration, and nX is a non-negatively charged internucleotidic linkage.

152. The oligonucleotide of Embodiment 151, wherein the pattern of stereochemistry and linkage of the oligonucleotide is or comprises: SOSSSSOOSSSSS, SOSSSSOOSS, SOSSSOSS, SOSSSOS, SnXSSSSSSSSSSnXSS, SnXSSOSSSOSSSnX, SnXSSOSSSOSSS, wherein O is a natural phosphate internucleotidic linkage, S is a phosphorothioate in the Sp configuration, and nX is a non-negatively charged internucleotidic linkage.

153. The oligonucleotide of any one of the preceding Embodiments, wherein the pattern of stereochemistry and linkage of the oligonucleotide is or comprises: SSSSSSSSnXSS, SSSSSSSS, SSSSSSS, SSSSSSnXSS, SSSSS, SSSSnXSS, SSSS, SSSOSSSS, SSSOOSSS, SSS, SSOSSSSOOS, SSOSSSOSS, SSOSSSOS, SSOSSSnX, SSOSSS, SSOOSS, SSOOS, SSnXSSnXSSOS, SSnXSS, SOSSSSOOSS, SOSSSOSSS, SOSSSOS, SOSSSOS, SOSSS, SOSS, SnXSSSSS, SnXSSOSSSOS, SnXSSnXS, OSSSOSSS, nXSSOSSSOS, nXSSOSS, wherein O is a natural phosphate internucleotidic linkage, S is a phosphorothioate in the Sp configuration, and nX is a non-negatively charged internucleotidic linkage.

154. The oligonucleotide of any one of the preceding Embodiments, wherein the pattern of stereochemistry and linkage of the oligonucleotide is or comprises: SSSSSSSSnXSS, SSSSSSSS, SSSSSSS, SSSSSSnXSS, SSSSS, SSSSnXSS, SSSS, SSSOSSSS, SSSOOSSS, SSS, SSOSSSSOOS, SSOSSSOSS, SSOSSSOS, SSOSSSnX, SSOSSS, SSOOSS, SSOOS, SSnXSSnXSSOS, SSnXSS, SOSSSSOOSS, SOSSSOSSS, SOSSSOS, SOSSSOS, SOSSS, SOSS, SnXSSSSS, SnXSSOSSSOS, SnXSSnXS, OSSSOSSS, nXSSOSSSOS, nXSSOSS, wherein O is a natural phosphate internucleotidic linkage, S is a phosphorothioate in the Sp configuration, and nX is a non-negatively charged internucleotidic linkage.

155. The oligonucleotide of any one of the preceding Embodiments, wherein the pattern of stereochemistry and linkage of the oligonucleotide is or comprises: SSSSSSSSnXSS, SSSSSSSS, SSSSSSS, SSSSSSnXSS, SSSSS, SSSSnXSS, SSSS, SSSOSSSS, SSSOOSSS, SSS, SSOSSSSOOS, SSOSSSOSS, SSOSSSOS, SSOSSSnX, SSOSSS, SSOOSS, SSOOS, SSnXSSnXSSOS, SSnXSS, SOSSSSOOSS, SOSSSOSSS, SOSSSOS, SOSSSOS, SOSSS, SOSS, SnXSSSSS, SnXSSOSSSOS, SnXSSnXS, OSSSOSSS, nXSSOSSSOS, nXSSOSS, wherein O is a natural phosphate internucleotidic linkage, S is a phosphorothioate in the Sp configuration, and nX is a non-negatively charged internucleotidic linkage.

156. The oligonucleotide of any one of the preceding Embodiments, wherein the pattern of stereochemistry and linkage of the oligonucleotide is or comprises: SSSSSSSSnXSS, SSSSSSSS, SSSSSSS, SSSSSSnXSS, SSSSS, SSSSnXSS, SSSS, SSSOSSSS, SSSOOSSS, SSS, SSOSSSSOOS, SSOSSSOSS, SSOSSSOS, SSOSSSnX, SSOSSS, SSOOSS, SSOOS, SSnXSSnXSSOS, SSnXSS, SOSSSSOOSS, SOSSSOSSS, SOSSSOS, SOSSSOS, SOSSS, SOSS, SnXSSSSS, SnXSSOSSSOS, SnXSSnXS, OSSSOSSS, nXSSOSSSOS, nXSSOSS, wherein O is a natural phosphate internucleotidic linkage, S is a phosphorothioate in the Sp configuration, and nX is a non-negatively charged internucleotidic linkage.

157. The oligonucleotide of any one of the preceding Embodiments, wherein the pattern of stereochemistry and linkage of the oligonucleotide is or comprises: SSSSSSSSnXSS, SSSSSSSS, SSSSSSS, SSSSSSnXSS, SSSSS, SSSSnXSS, SSSS, SSSOSSSS, SSSOOSSS, SSS, SSOSSSSOOS, SSOSSSOSS, SSOSSSOS, SSOSSSnX, SSOSSS, SSOOSS, SSOOS, SSnXSSnXSSOS, SSnXSS, SOSSSSOOSS, SOSSSOSSS, SOSSSOS, SOSSSOS, SOSSS, SOSS, SnXSSSSS, SnXSSOSSSOS, SnXSSnXS, OSSSOSSS, nXSSOSSSOS, nXSSOSS, wherein O is a natural phosphate internucleotidic linkage, S is a phosphorothioate in the Sp configuration, and nX is a non-negatively charged internucleotidic linkage.

158. The oligonucleotide of any one of the preceding Embodiments, wherein the pattern of stereochemistry and linkage of the oligonucleotide is or comprises: SSSSSSSSnXSS, SSSSSSSS, SSSSSSS, SSSSSSnXSS, SSSSS, SSSSnXSS, SSSS, SSSOSSSS, SSSOOSSS, SSS, SSOSSSSOOS, SSOSSSOSS, SSOSSSOS, SSOSSSnX, SSOSSS, SSOOSS, SSOOS, SSnXSSnXSSOS, SSnXSS, SOSSSSOOSS, SOSSSOSSS, SOSSSOS, SOSSSOS, SOSSS, SOSS, SnXSSSSS, SnXSSOSSSOS, SnXSSnXS, OSSSOSSS, nXSSOSSSOS, nXSSOSS, wherein O is a natural phosphate internucleotidic linkage, S is a phosphorothioate in the Sp configuration, and nX is a non-negatively charged internucleotidic linkage.

159. The oligonucleotide of Embodiment 108, wherein the pattern of stereochemistry and linkage of the oligonucleotide is or comprises: SSOSSSOSS, SSOSSSOS, SSOSSSnX, SSOSSS, SSOOSS, SSOOS, SSnXSSnXSSOS, SSnXSS, SOSSSSOOSS, SOSSSOSSS, SOSSSOS, SOSSSOS, SOSSS, SOSS, SnXSSSSS, SnXSSOSSSOS, SnXSSnXS, OSSSOSSS, nXSSOSSSOS, nXSSOSS, wherein O is a natural phosphate internucleotidic linkage, S is a phosphorothioate in the Sp configuration, and nX is a non-negatively charged internucleotidic linkage.

160. The oligonucleotide of any one of the preceding Embodiments, wherein the pattern of stereochemistry and linkage of the oligonucleotide is or comprises: SSSOOSSS, SSS, SSOSSSSOOS, SSOSSSOSS, SSOSSSOS, SSOSSSnX, SSOSSS, SSOOSS, SSOOS, SSnXSSnXSSOS, SSnXSS, SOSSSSOOSS, SOSSSOSSS, SOSSSOS, SOSSSOS, SOSSS, SOSS, SnXSSSSS, SnXSSOSSSOS, SnXSSnXS, OSSSOSSS, nXSSOSSSOS, nXSSOSS, wherein O is a natural phosphate internucleotidic linkage, S is a phosphorothioate in the Sp configuration, and nX is a non-negatively charged internucleotidic linkage.

161. The oligonucleotide of any one of the preceding Embodiments, wherein the pattern of stereochemistry and linkage of the oligonucleotide is or comprises: SSSSSSSSnXSS, SSSSSSSS, SSSSSSS, SSSSSSnXSS, SSSSS, SSSSnXSS, SSSS, SSSOSSSS, SSSOOSSS, SnXSSOSSSOS, SnXSSnXS, OSSSOSSS, nXSSOSSSOS, nXSSOSS, wherein O is a natural phosphate internucleotidic linkage, S is a phosphorothioate in the Sp configuration, and nX is a non-negatively charged internucleotidic linkage.

162. The oligonucleotide of any one of the preceding Embodiments, wherein the pattern of stereochemistry and linkage of the oligonucleotide is or comprises: SSSSSSSSnXSS, SSSSSSSS, SSSSSSS, SSSSSSnXSS, SSSSS, SSSSnXSS, SSSS, SSSOSSSS, SSSOOSSS, SSS, SSOSSSSOOS, SSOSSSOSS, SSOSSSOS, SSOSSSnX, SSOSSS, SOSS, SnXSSSSS, SnXSSOSSSOS, SnXSSnXS, OSSSOSSS, nXSSOSSSOS, nXSSOSS, wherein O is a natural phosphate internucleotidic linkage, S is a phosphorothioate in the Sp configuration, and nX is a non-negatively charged internucleotidic linkage.

163. The oligonucleotide of any one of the preceding Embodiments, wherein the pattern of stereochemistry and linkage of the oligonucleotide is or comprises: SSSSSSSSnXSS, SSSSSSSS, SSSSSSS, SSSSSSnXSS, SSSSS, SSSSnXSS, SSSS, SSSOSSSS, SSSOOSSS, SSS, SSOSSSSOOS, SSOSSSOSS, SSOSSSOS, SSOSSSnX, SSOSSS, SSOOSS, wherein O is a natural phosphate internucleotidic linkage, S is a phosphorothioate in the Sp configuration, and nX is a non-negatively charged internucleotidic linkage.

164. The oligonucleotide of any one of the preceding Embodiments, wherein the pattern of stereochemistry and linkage of the oligonucleotide is or comprises: SSSSSSSSnXSS, SSSSSSSS, SSSSSSS, SSSSSSnXSS, SSSSS, SSSSnXSS, SSSS, SSSOSSSS, SSnXSSnXSSOS, SSnXSS, SOSSSSOOSS, SOSSSOSSS, SOSSSOS, SOSSSOS, wherein O is a natural phosphate internucleotidic linkage, S is a phosphorothioate in the Sp configuration, and nX is a non-negatively charged internucleotidic linkage.

165. The oligonucleotide of any one of Embodiments 1-113, wherein the pattern of stereochemistry and linkage of the oligonucleotide is or comprises: SSSSSSSSnXSS, SSOSSSOSS, SSOSSSOS, SSOSSSnX, SSOSSS, SSOOSS, SSOOS, SSnXSSnXSSOS, SSnXSS, SOSSSSOOSS, SOSSSOSSS, SOSSSOS, SOSSSOS, wherein O is a natural phosphate internucleotidic linkage, S is a phosphorothioate in the Sp configuration, and nX is a non-negatively charged internucleotidic linkage.

166. The oligonucleotide of any one of the preceding Embodiments, wherein the pattern of stereochemistry and linkage of the oligonucleotide is or comprises: SSSSSSSSnXSS, SSnXSS, SOSSSSOOSS, SOSSSOSSS, SOSSSOS, SOSSSOS, SOSSS, SOSS, SnXSSSSS, SnXSSOSSSOS, wherein O is a natural phosphate internucleotidic linkage, S is a phosphorothioate in the Sp configuration, and nX is a non-negatively charged internucleotidic linkage.

167. The oligonucleotide of any one of the preceding Embodiments, wherein the pattern of stereochemistry and linkage of the oligonucleotide is or comprises: SSSSSSSSnXSS, SSnXSS, SOSSSSOOSS, SOSSSOSSS, SOSSSOS, SOSSSOS, wherein O is a natural phosphate internucleotidic linkage, S is a phosphorothioate in the Sp configuration, and nX is a non-negatively charged internucleotidic linkage.

168. The oligonucleotide of any one of the preceding Embodiments, wherein the pattern of stereochemistry and linkage of the oligonucleotide is or comprises: SSnXSSnXSSOS, SSnXSS, SOSSSSOOSS, SOSSSOSSS, SOSSSOS, SOSSSOS, SOSSS, SOSS, SnXSSSSS, SnXSSOSSSOS, SnXSSnXS, wherein O is a natural phosphate internucleotidic linkage, S is a phosphorothioate in the Sp configuration, and nX is a non-negatively charged internucleotidic linkage.

169. The oligonucleotide of any one of the preceding Embodiments, wherein the pattern of stereochemistry and linkage of the oligonucleotide is or comprises: SSnXSSnXSSOS, SSnXSS, SOSSSSOOSS, SOSSSOSSS, SOSSSOS, SOSSSOS, SOSSS, SOSS, SnXSSSSS, SnXSSOSSSOS, SnXSSnXS, OSSSOSSS, nXSSOSSSOS, nXSSOSS, wherein O is a natural phosphate internucleotidic linkage, S is a phosphorothioate in the Sp configuration, and nX is a non-negatively charged internucleotidic linkage.

170. The oligonucleotide of any one of the preceding Embodiments, wherein the pattern of stereochemistry and linkage of the oligonucleotide is or comprises: SSSSSSSSnXSS, SSSSSSSS, SSSSSSS, SSSSSSnXSS, SSSSS, SSSSnXSS, SSSS, SSSOSSSS, SSSOOSSS, SSS, SSOSSSSOOS, SSOSSSOSS, SSOSSSOS, SSOSSSnX, SSOSSS, wherein O is a natural phosphate internucleotidic linkage, S is a phosphorothioate in the Sp configuration, and nX is a non-negatively charged internucleotidic linkage.

171. The oligonucleotide of any one of Embodiments 1-119, wherein the pattern of stereochemistry and linkage of the oligonucleotide is or comprises: SSSSSSSS, SSSSSSS, SSSSSSnXSS, SSSSS, SSSSnXSS, SSSS, SSSOSSSS, SSSOOSSS, SSS, SnXSSOSSSOS, SnXSSnXS, OSSSOSSS, nXSSOSSSOS, nXSSOSS, wherein O is a natural phosphate internucleotidic linkage, S is a phosphorothioate in the Sp configuration, and nX is a non-negatively charged internucleotidic linkage.

172. The oligonucleotide of any one of the preceding Embodiments, wherein the pattern of stereochemistry and linkage of the oligonucleotide is or comprises: SSSSSSSSnXSS, SSSSSSSS, SSSSSSS, SSSSSSnXSS, SSSSS, SSSSnXSS, SSSS, SSSOSSSS, SSSOOSSS, SSnXSS, SOSSSSOOSS, SOSSSOSSS, SOSSSOS, SOSSSOS, SOSSS, SOSS, SnXSSSSS, SnXSSOSSSOS, SnXSSnXS, OSSSOSSS, nXSSOSSSOS, nXSSOSS, wherein O is a natural phosphate internucleotidic linkage, S is a phosphorothioate in the Sp configuration, and nX is a non-negatively charged internucleotidic linkage.

173. The oligonucleotide of any one of the preceding Embodiments, wherein one or more nucleosides comprising a 2′-OMe modification are independently bonded to one or two Sp chiral internucleotidic linkages.

174. The oligonucleotide of any one of the preceding Embodiments, wherein one or more nucleosides comprising a high affinity sugar are independently bonded to one or two natural phosphate linkages.

175. The oligonucleotide of any one of the preceding Embodiments, wherein one or more nucleosides comprising a high affinity sugar are independently bonded to one or two neutral internucleotidic linkages.

176. The oligonucleotide of any one of the preceding Embodiments, wherein one or more nucleosides comprising a 2′-MOE modification are independently bonded to one or two natural phosphate linkages.

177. The oligonucleotide of any one of the preceding Embodiments, wherein one or more nucleosides comprising a 2′-MOE modification are independently bonded to one or two neutral internucleotidic linkages.

178. The oligonucleotide of any one of the preceding Embodiments, wherein the pattern of backbone chiral centers of the oligonucleotide comprises or consists of (Np)t[(Op/Rp)n(Sp)m]y, wherein:
t is 1-50;
n is 1-10;
m is 1-50;
y is 1-10;
each Np is independently Rp or Sp;
Op indicates a linkage phosphorus being achiral;
Rp indicates a linkage phosphorus having R configuration; and
Sp indicates a linkage phosphorus having S configuration.

179. The oligonucleotide of any one of the preceding Embodiments, wherein the pattern of backbone chiral centers of the oligonucleotide comprises or consists of (Np)t[(Op/Rp)n(Sp)m]yRp, wherein:
t is 1-50;
n is 1-10;
m is 1-50;
y is 1-10;
each Np is independently Rp or Sp;
Op indicates a linkage phosphorus being achiral;
Rp indicates a linkage phosphorus having R configuration; and
Sp indicates a linkage phosphorus having S configuration.

180. The oligonucleotide of Embodiment 178 or 179, wherein at least one Np is Sp.

181. The oligonucleotide of Embodiment 178 or 179, wherein (Np)t is Rp(Np)t-1.

182. The oligonucleotide of Embodiment 178 or 179, wherein (Np)t is Rp(Sp)t-1.

183. The oligonucleotide of Embodiment 180, wherein each Np is Sp.

184. The oligonucleotide of any one of Embodiments 178-183, wherein at least one (Op/Rp) is Rp.

185. The oligonucleotide of any one of Embodiments 178-184, wherein each (Op/Rp) is Rp.

186. The oligonucleotide of any one of Embodiments 178-185, wherein at least one n is 1.

187. The oligonucleotide of any one of Embodiments 178-185, wherein each n is 1.

188. The oligonucleotide of any one of Embodiments 178-187, wherein t is 2, 3, 4, 5, 6, 7, 8, 9 or 10.

189. The oligonucleotide of any one of Embodiments 178-188, wherein at least one m is 2-50.

190. The oligonucleotide of any one of Embodiments 178-188, wherein each m is independently 2, 3, 4, 5, 6, 7, 8, 9 or 10.

191. The oligonucleotide of any one of Embodiments 178-188, wherein at least one m is 1.

192. The oligonucleotide of any one of Embodiments 178-188, wherein one m is 1, and each other m is independently 2-50.

193. The oligonucleotide of any one of Embodiments 178-192, wherein y is 1.

194. The oligonucleotide of any one of Embodiments 178-192, wherein y is 2, 3, 4 or 5.

195. The oligonucleotide of any one of Embodiments 178-192, wherein the pattern of backbone chiral centers comprises OpSpOpSpSp.

196. The oligonucleotide of any one of Embodiments 178-192, wherein the pattern of backbone chiral centers comprises (Sp)tOpSpOpSpSp.

197. The oligonucleotide of any one of Embodiments 178-196, wherein the first Np of (Np)t in (Np)t[(Op/Rp)n(Sp)m]y or (Np)t[(Op/Rp)n(Sp)m]yRp represents linkage phosphorus stereochemistry of the first internucleotidic linkage of the oligonucleotide from 5′ to 3′.

198. The oligonucleotide of any one of Embodiments 178-197, wherein the last Sp of (Np)t[(Op/Rp)n(Sp)m]y or the last Rp of (Np)t[(Op/Rp)n(Sp)m]yRp represents linkage phosphorus stereochemistry of the last internucleotidic linkage of the oligonucleotide from 5′ to 3′.

199. The oligonucleotide of any one of the preceding Embodiments, wherein USH2A oligonucleotide mediates skipping of exon 13 of an USH2A gene transcript.

200. The oligonucleotide of Embodiment 199, wherein USH2A oligonucleotide mediates skipping of exon 13 of an USH2A gene transcript, and wherein exon 13 comprises a deleterious mutation.

201. The oligonucleotide of any one of the preceding Embodiments, wherein USH2A oligonucleotide mediates skipping of exon 13 of an USH2A gene transcript, and wherein exon 13 comprises a deleterious mutation selected from: 2299delG, c.2802T>G, c.2776C>T, c.2761delC, c.2541C>A, c.2522C>A, c.2276G>T, and c.2242C>T.

202. The oligonucleotide of any one of the preceding Embodiments, wherein USH2A oligonucleotide mediates skipping of exon 13 of an USH2A gene transcript, and wherein exon 13 comprises a deleterious mutation of 2299delG.

203. The oligonucleotide of any one of the preceding Embodiments, wherein USH2A oligonucleotide mediates skipping of exon 13 of an USH2A gene transcript, and wherein exon 13 comprises a deleterious mutation selected from: 2299delG or c.2802T>G.

204. The oligonucleotide of any one of the preceding Embodiments, wherein USH2A oligonucleotide mediates skipping of exon 13 of an USH2A gene transcript, and wherein exon 13 comprises a deleterious mutation selected from: 2299delG, c.2802T>G, or c.2776C>T.

205. The oligonucleotide of any one of the preceding Embodiments, wherein USH2A oligonucleotide mediates skipping of exon 13 of an USH2A gene transcript, and wherein exon 13 comprises a deleterious mutation which is c.2802T>G.

206. The oligonucleotide of any one of the preceding Embodiments, wherein USH2A oligonucleotide mediates skipping of exon 13 of an USH2A gene transcript, and wherein exon 13 comprises a deleterious mutation which is c.2761delC.

207. The oligonucleotide of any one of the preceding Embodiments, wherein USH2A oligonucleotide mediates skipping of exon 13 of an USH2A gene transcript, and wherein exon 13 comprises a deleterious mutation which is c.2522C>A.

208. The oligonucleotide of any one of the preceding Embodiments, wherein USH2A oligonucleotide mediates skipping of exon 13 of an USH2A gene transcript, and wherein exon 13 comprises a deleterious mutation which is c.2276G>T 209. The oligonucleotide of any one of the preceding Embodiments, wherein USH2A oligonucleotide mediates skipping of exon 13 of an USH2A gene transcript, and wherein exon 13 comprises a deleterious mutation which is c.2242C>T.

210. The oligonucleotide of any one of the preceding Embodiments, wherein the oligonucleotide is capable of mediating at least 30% of skipping of exon 13 in vitro at a concentration of around 50 uM.

211. The oligonucleotide of any one of the preceding Embodiments, wherein the oligonucleotide is capable of mediating at least 40% of skipping of exon 13 in vitro at a concentration of around 50 uM.

212. The oligonucleotide of any one of the preceding Embodiments, wherein the oligonucleotide is capable of mediating at least 50% of skipping of exon 13 in vitro at a concentration of around 50 uM.

213. The oligonucleotide of any one of the preceding Embodiments, wherein the oligonucleotide is capable of mediating at least 60% of skipping of exon 13 in vitro at a concentration of around 50 uM.

214. The oligonucleotide of any one of the preceding Embodiments, wherein the oligonucleotide is capable of mediating at least 70% of skipping of exon 13 in vitro at a concentration of around 50 uM.

215. The oligonucleotide of any one of the preceding Embodiments, wherein the oligonucleotide is capable of mediating at least 70% of skipping of exon 13 in vitro at a concentration of around 50 uM.

216. The oligonucleotide of any one of the preceding Embodiments, wherein the oligonucleotide is capable of mediating at least 30% of skipping of exon 13 in vitro at a concentration of around 50 uM in a Y-79 cell.

217. The oligonucleotide of any one of the preceding Embodiments, wherein the oligonucleotide is capable of mediating at least 40% of skipping of exon 13 in vitro at a concentration of around 50 uM in a Y-79 cell.

218. The oligonucleotide of any one of the preceding Embodiments, wherein the oligonucleotide is capable of mediating at least 50% of skipping of exon 13 in vitro at a concentration of around 50 uM in a Y-79 cell.

219. The oligonucleotide of any one of the preceding Embodiments, wherein the oligonucleotide is capable of mediating at least 60% of skipping of exon 13 in vitro at a concentration of around 50 uM in a Y-79 cell.

220. The oligonucleotide of any one of the preceding Embodiments, wherein the oligonucleotide is capable of mediating at least 70% of skipping of exon 13 in vitro at a concentration of around 50 uM in a Y-79 cell.

221. The oligonucleotide of any one of Embodiments 210-220, wherein the condition, disorder or disease is Usher Syndrome (e.g., Usher Syndrome Type 2A), atypical Usher syndrome, or nonsyndromic retinitis pigmentosa.

222. The oligonucleotide of any one of Embodiments 210-220, wherein the condition, disorder or disease is retinitis pigmentosa.

223. The oligonucleotide of any one of Embodiments 210-220, wherein the condition, disorder or disease is Usher Syndrome (e.g., Usher Syndrome Type 2A) 224. The oligonucleotide of any one of Embodiments 210-220, wherein the condition, disorder or disease is atypical Usher syndrome.

225. The oligonucleotide of any one of Embodiments 205-224, wherein the Rp is an Rp in the pattern of backbone chiral centers of any one of Embodiments 178-198.

226. The oligonucleotide of any one of the preceding Embodiments, wherein the oligonucleotide comprises one or more non-negatively charged internucleotidic linkages.

227. The oligonucleotide of any one of the preceding Embodiments, wherein the oligonucleotide comprises one or more non-negatively charged internucleotidic linkages having the structure of formula I, I-a-1, I-a-2, I-b, I-c, I-d, I-e, I-n-1, I-n-2, I-n-3, I-n-4, II, II-a-1, II-a-2, II-b-1, II-b-2, II-c-1, II-c-2, II-d-1, or II-d-2.

228. The oligonucleotide of any one of the preceding Embodiments, wherein the oligonucleotide comprises one or more neutral internucleotidic linkages.

229. The oligonucleotide of any one of the preceding Embodiments, wherein the oligonucleotide comprises one or more neutral internucleotidic linkages having the structure of formula I, I-a-1, I-a-2, I-b, I-c, I-d, I-e, I-n-1, I-n-2, I-n-3, I-n-4, II, II-a-1, II-a-2, II-b-1, II-b-2, II-c-1, II-c-2, II-d-1, or II-d-2.

230. The oligonucleotide of any one of Embodiments 226-229, wherein the non-negatively charged internucleotidic linkage or neutral internucleotidic linkage is n001.

231. The oligonucleotide of any one of the preceding Embodiments, wherein each non-negatively charged internucleotidic linkage or neutral internucleotidic linkage is n001.

232. The oligonucleotide of any one of Embodiments 226-230, wherein the non-negatively charged internucleotidic linkage or neutral internucleotidic linkage is chirally controlled.

233. The oligonucleotide of any one of Embodiments 226-232, wherein the oligonucleotide comprises two or more adjacent non-negatively charged internucleotidic linkages.

234. The oligonucleotide of any one of the preceding Embodiments, wherein each internucleotidic linkage independently has the structure of formula I, I-a-1, I-a-2, I-b, I-c, I-d, I-e, I-n-1, I-n-2, I-n-3, I-n-4, II, II-a-1, II-a-2, II-b-1, II-b-2, II-c-1, II-c-2, II-d-1, or II-d-2, or a salt form thereof.

235. The oligonucleotide of any one of the preceding Embodiments, wherein the first region comprises one or more (e.g., 1, 2, 3, 4, 5, 6, 7, 8, 9, 10 or more) phosphorothioate internucleotidic linkages.

236. The oligonucleotide of any one of the preceding Embodiments, wherein the first region comprises four or more phosphorothioate internucleotidic linkages.

237. The oligonucleotide of any one of the preceding Embodiments, wherein at least 50%, 60%, 70%, 75%, 80%, 85%, 90%, or 95%, or 100% of all internucleotidic linkages in the first region are phosphorothioate internucleotidic linkages.

238. The oligonucleotide of any one of the preceding Embodiments, wherein at least 50%, 60%, 70%, 75%, 80%, 85%, 90%, or 95%, or 100% of all phosphorothioate internucleotidic linkages in the first region are Sp.

239. The oligonucleotide of any one of the preceding Embodiments, wherein all phosphorothioate internucleotidic linkages in the first region are Sp.

240. The oligonucleotide of any one of the preceding Embodiments, wherein two or more phosphorothioate linkages in a first region are consecutive.

241. The oligonucleotide of any one of the preceding Embodiments, where the first region comprises one or more (e.g., 1, 2, 3, 4, 5, 6, 7, 8, 9, 10 or more) non-negatively charged internucleotidic linkages.

242. The oligonucleotide of any one of the preceding Embodiments, wherein at least 50%, 60%, 70%, 75%, 80%, 85%, 90%, or 95%, or 100% of all non-negatively charged internucleotidic linkages in the first region are Rp.

243. The oligonucleotide of any one of the preceding Embodiments, wherein all non-negatively charged internucleotidic linkages in the first region are Rp.

244. The oligonucleotide of any one of the preceding Embodiments, wherein a first region comprises one or more natural phosphate linkages.

245. The oligonucleotide of any one of Embodiments 235-243, wherein each internucleotidic linkage in a first region is independently a phosphorothioate internucleotidic linkage or a non-negatively charged internucleotidic linkage.

246. The oligonucleotide of any one of the preceding Embodiments, wherein the second region comprises one or more (e.g., 1, 2, 3, 4, 5, 6, 7, 8, 9, 10 or more) phosphorothioate internucleotidic linkages.

247. The oligonucleotide of any one of the preceding Embodiments, wherein the second region comprises two or more phosphorothioate internucleotidic linkages.

248. The oligonucleotide of any one of the preceding Embodiments, wherein at least 50%, 60%, 70%, 75%, 80%, 85%, 90%, or 95%, or 100% of all internucleotidic linkages in the second region are phosphorothioate internucleotidic linkages.

249. The oligonucleotide of any one of the preceding Embodiments, wherein at least 50%, 60%, 70%, 75%, 80%, 85%, 90%, or 95%, or 100% of all phosphorothioate internucleotidic linkages in the second region are Sp.

250. The oligonucleotide of any one of the preceding Embodiments, wherein all phosphorothioate internucleotidic linkages in the second region are Sp.

251. The oligonucleotide of any one of the preceding Embodiments, wherein two or more phosphorothioate linkages in a second region are consecutive.

252. The oligonucleotide of any one of the preceding Embodiments, where the second region comprises one or more (e.g., 1, 2, 3, 4, 5, 6, 7, 8, 9, 10 or more) non-negatively charged internucleotidic linkages.

253. The oligonucleotide of any one of the preceding Embodiments, wherein at least 50%, 60%, 70%, 75%, 80%, 85%, 90%, or 95%, or 100% of all non-negatively charged internucleotidic linkages in the second region are Rp.

254. The oligonucleotide of any one of the preceding Embodiments, wherein all non-negatively charged internucleotidic linkages in the second region are Rp.

255. The oligonucleotide of any one of the preceding Embodiments, wherein a second region comprises one or more natural phosphate linkages.

256. The oligonucleotide of any one of Embodiments 235-254, wherein each internucleotidic linkage in a second region is independently a phosphorothioate internucleotidic linkage or a non-negatively charged internucleotidic linkage.

257. The oligonucleotide of any one of the preceding Embodiments, wherein the third region comprises one or more (e.g., 1, 2, 3, 4, 5, 6, 7, 8, 9, 10 or more) phosphorothioate internucleotidic linkages.

258. The oligonucleotide of any one of the preceding Embodiments, wherein the third region comprises four or more phosphorothioate internucleotidic linkages.

259. The oligonucleotide of any one of the preceding Embodiments, wherein at least 50%, 60%, 70%, 75%, 80%, 85%, 90%, or 95%, or 100% of all internucleotidic linkages in the third region are phosphorothioate internucleotidic linkages.

260. The oligonucleotide of any one of the preceding Embodiments, wherein at least 50%, 60%, 70%, 75%, 80%, 85%, 90%, or 95%, or 100% of all phosphorothioate internucleotidic linkages in the third region are Sp.

261. The oligonucleotide of any one of the preceding Embodiments, wherein all phosphorothioate internucleotidic linkages in the third region are Sp.

262. The oligonucleotide of any one of the preceding Embodiments, wherein two or more phosphorothioate linkages in a third region are consecutive.

263. The oligonucleotide of any one of the preceding Embodiments, where the third region comprises one or more (e.g., 1, 2, 3, 4, 5, 6, 7, 8, 9, 10 or more) non-negatively charged internucleotidic linkages.

264. The oligonucleotide of any one of the preceding Embodiments, wherein at least 50%, 60%, 70%, 75%, 80%, 85%, 90%, or 95%, or 100% of all non-negatively charged internucleotidic linkages in the third region are Rp.

265. The oligonucleotide of any one of the preceding Embodiments, wherein all non-negatively charged internucleotidic linkages in the third region are Rp.

266. The oligonucleotide of any one of the preceding Embodiments, wherein a third region comprises one or more natural phosphate linkages.

267. The oligonucleotide of any one of Embodiments 235-265, wherein each internucleotidic linkage in a third region is independently a phosphorothioate internucleotidic linkage or a non-negatively charged internucleotidic linkage.

268. The oligonucleotide of any one of the preceding Embodiments, wherein each internucleotidic linkage is independently selected from natural phosphate linkages, phosphorothioate internucleotidic linkages and non-negatively charged internucleotidic linkages.

269. The oligonucleotide of any one of the preceding Embodiments, wherein each internucleotidic linkage is independently selected from natural phosphate linkages, phosphorothioate internucleotidic linkages and n001.

270. The oligonucleotide of any one of Embodiments 1-225, wherein each internucleotidic linkage is independently selected from a natural phosphate linkage and a phosphorothioate internucleotidic linkage.

271. The oligonucleotide of any one of the preceding Embodiments, wherein each chiral internucleotidic linkage of the oligonucleotide independently comprises a stereodefined linkage phosphorus.

272. The oligonucleotide of any one of the preceding Embodiments, wherein each nucleobase of the oligonucleotide is independently optionally substituted A, 2AP, DAP, T, C, G or U, or an optionally substituted tautomer of A, T, C, G or U.

273. The oligonucleotide of any one of the preceding Embodiments, wherein each nucleobase of the oligonucleotide is independently optionally substituted A, T, C, G or U, or an optionally substituted tautomer of A, T, C, G or U.

274. The oligonucleotide of any one of the preceding Embodiments, wherein the oligonucleotide chain is conjugated with a lipid moiety, a carbohydrate moiety, or a targeting moiety.

275. An oligonucleotide selected from Table A1 or a salt form thereof.

276. An oligonucleotide, wherein the oligonucleotide is WV-30205, or a salt form thereof.

277. An oligonucleotide, wherein the oligonucleotide is WV-36863, or a salt form thereof.

278. An oligonucleotide, wherein the oligonucleotide is WV-36868, or a salt form thereof.

279. An oligonucleotide, wherein the oligonucleotide is WV-36865, or a salt form thereof.

280. An oligonucleotide, wherein the oligonucleotide is WV-20891, WV-20892, WV-20902, WV-20908, WV-20988, or WV-21008, or a salt form thereof.

281. An oligonucleotide, wherein the oligonucleotide is WV-20891, or a salt form thereof.

282. An oligonucleotide, wherein the oligonucleotide is WV-20892, or a salt form thereof.

283. An oligonucleotide, wherein the oligonucleotide is WV-20902, or a salt form thereof.

284. An oligonucleotide, wherein the oligonucleotide is WV-20908, or a salt form thereof.

285. An oligonucleotide, wherein the oligonucleotide is WV-20988, or a salt form thereof.

286. An oligonucleotide, wherein the oligonucleotide is WV-21008, or a salt form thereof.

287. An oligonucleotide, wherein the oligonucleotide is WV-20885, or a salt form thereof.

288. An oligonucleotide, wherein the oligonucleotide is WV-21008, or a salt form thereof.

289. An oligonucleotide, wherein the oligonucleotide is WV-21100, or a salt form thereof.

290. An oligonucleotide, wherein the oligonucleotide is WV-21105, or a salt form thereof.

291. An oligonucleotide, wherein the oligonucleotide is WV-24297, or a salt form thereof.

292. An oligonucleotide, wherein the oligonucleotide is WV-24298, or a salt form thereof.

293. An oligonucleotide, wherein the oligonucleotide is WV-24360, or a salt form thereof.

294. An oligonucleotide, wherein the oligonucleotide is WV-24366, or a salt form thereof.

295. An oligonucleotide, wherein the oligonucleotide is WV-24368, or a salt form thereof.

296. An oligonucleotide, wherein the oligonucleotide is WV-24375, or a salt form thereof.

297. An oligonucleotide, wherein the oligonucleotide is WV-24376, or a salt form thereof.

298. An oligonucleotide, wherein the oligonucleotide is WV-24381, or a salt form thereof.

299. An oligonucleotide, wherein the oligonucleotide is WV-24382, or a salt form thereof.

300. The oligonucleotide of any one of the preceding Embodiments, wherein the oligonucleotide is in a form of a pharmaceutically acceptable salt.

301. The oligonucleotide of any one of the preceding Embodiments, wherein the oligonucleotide is a sodium salt form.

302. The oligonucleotide of any one of the preceding Embodiments, wherein each phosphorothioate internucleotidic linkage of the oligonucleotide independently has a diastereomeric purity of at least 50%, 60%, 70%, 80%, 85%, 90%, 95%, 96%, 97%, 98%, or 99%.

303. The oligonucleotide of any one of the preceding Embodiments, wherein each chiral internucleotidic linkage of the oligonucleotide independently has a diastereomeric purity of at least 50%, 60%, 70%, 80%, 85%, 90%, 95%, 96%, 97%, 98%, or 99%.

304. The oligonucleotide of any one of the preceding Embodiments, wherein the oligonucleotide has a diastereomeric purity of at least 50%, 60%, 70%, 80%, 85%, 90%, 95%, 96%, 97%, 98%, or 99%.

305. A chirally controlled oligonucleotide composition comprising a plurality of oligonucleotides, wherein the oligonucleotides share:
1) a common base sequence,
2) a common pattern of backbone linkages, and
3) a common pattern of backbone chiral center,
wherein the composition is enriched, relative to a substantially racemic preparation of oligonucleotides sharing the common base sequence and pattern of backbone linkages, for oligonucleotides of the plurality, and
each oligonucleotide of the plurality is independently an oligonucleotide of any one of Embodiments 1-301.

306. The composition of Embodiment 305, wherein 5%, 10%, 15%, 20%, 25%, 30%, 35%, 40%, 45%, 50%, 60%, 70%, 75%, 80%, 85%, 90%, 95%, 96%, 97%, 98%, 99% or more of all oligonucleotides within the composition that share the common base sequence and common pattern of backbone linkages are the oligonucleotides of the plurality.

307. A chirally controlled oligonucleotide composition comprising a plurality of oligonucleotides, wherein the oligonucleotides share:
1) a common base sequence,
2) a common pattern of backbone linkages, and
3) the same linkage phosphorus stereochemistry at one or more (e.g., 1, 2, 3, 4, 5, 6, 7, 8, 9, 10, 11, 12, 13, 14, 15, 16, 17, 18, 19, 20 or more) chiral internucleotidic linkages (chirally controlled internucleotidic linkages),
wherein about 1-100% of all oligonucleotides within the composition that share the common base sequence and common pattern of backbone linkages are the oligonucleotides of the plurality,
each oligonucleotide of the plurality is independently an oligonucleotide of any one of Embodiments 1-301.

308. The composition of any one of the preceding Embodiments, wherein oligonucleotides of the plurality share the same constitution.

309. A chirally controlled oligonucleotide composition comprising a plurality of oligonucleotides, wherein the oligonucleotides share:
1) a common constitution, and
2) the same linkage phosphorus stereochemistry at one or more (e.g., 1, 2, 3, 4, 5, 6, 7, 8, 9, 10, 11, 12, 13, 14, 15, 16, 17, 18, 19, 20 or more) chiral internucleotidic linkages (chirally controlled internucleotidic linkages),
wherein the composition is enriched, relative to a substantially racemic preparation of oligonucleotides sharing the common constitution, for oligonucleotides of the plurality, and
each oligonucleotide of the plurality is independently an oligonucleotide of any one of Embodiments 1-301.

310. The composition of Embodiment 309, wherein 5%, 10%, 15%, 20%, 25%, 30%, 35%, 40%, 45%, 50%, 60%, 70%, 75%, 80%, 85%, 90%, 95%, 96%, 97%, 98%, 99% or more of all oligonucleotides within the composition that share the common constitution are the oligonucleotides of the plurality.

311. A chirally controlled oligonucleotide composition comprising a plurality of oligonucleotides, wherein the oligonucleotides share:
1) a common constitution, and
2) the same linkage phosphorus stereochemistry at one or more (e.g., 1, 2, 3, 4, 5, 6, 7, 8, 9, 10, 11, 12, 13, 14, 15, 16, 17, 18, 19, 20 or more) chiral internucleotidic linkages (chirally controlled internucleotidic linkages),
wherein about 1-100% of all oligonucleotides within the composition that share the common constitution are the oligonucleotides of the plurality, and
each oligonucleotide of the plurality is independently an oligonucleotide of any one of Embodiments 1-301.

312. The composition of any one of the preceding Embodiments, wherein oligonucleotides of the plurality share the same linkage phosphorus stereochemistry at 5 or more chiral internucleotidic linkages.

313. The composition of any one of the preceding Embodiments, wherein oligonucleotides of the plurality share the same linkage phosphorus stereochemistry independently at each phosphorothioate internucleotidic linkage.

314. The composition of any one of the preceding Embodiments, wherein oligonucleotides of the plurality share the same Sp linkage phosphorus stereochemistry independently at each phosphorothioate internucleotidic linkage.

315. The composition of any one of the preceding Embodiments, wherein oligonucleotides of the plurality share the same linkage phosphorus stereochemistry independently at each non-negatively charged internucleotidic linkage.

316. The composition of any one of the preceding Embodiments, wherein oligonucleotides of the plurality share the same Rp linkage phosphorus stereochemistry independently at each non-negatively charged internucleotidic linkage.

317. The composition of any one of the preceding Embodiments, wherein about 1-100% of all oligonucleotides within the composition that share the common base sequence are oligonucleotides of the plurality.

318. The composition of any one of the preceding Embodiments, wherein the percentage is 5%, 10%, 15%, 20%, 25%, 30%, 35%, 40%, 45%, 50%, 60%, 70%, 75%, 80%, 85%, 90%, 95%, 96%, 97%, 98%, 99% or more.

319. The composition of any one of the preceding Embodiments, wherein the percentage is 50% or more.

320. The composition of any one of the preceding Embodiments, wherein substantially all oligonucleotides within the composition that share the common base sequence and common pattern of backbone linkages are the oligonucleotides of the plurality.

321. The composition of any one of the preceding Embodiments, wherein oligonucleotides of the plurality are identical.

322. The composition of Embodiment 321, wherein the composition is substantially free of other stereoisomeric forms of the oligonucleotides.

323. The composition of any one of Embodiments 218-224, wherein oligonucleotides of the plurality are each a pharmaceutically acceptable salt.

324. The composition of any one of Embodiments 218-225, wherein oligonucleotides of the plurality are each a sodium salt.

325. The composition of any one of Embodiments 218-225, wherein oligonucleotides of the plurality are of two or more pharmaceutically acceptable salts.

326. A pharmaceutical composition comprising or delivering an oligonucleotide or a composition of any one of the preceding Embodiments and a pharmaceutically acceptable carrier.

327. The composition of Embodiment 326, wherein the oligonucleotide is a pharmaceutically acceptable salt form.

328. The composition of Embodiment 327, wherein the oligonucleotide is a sodium salt form.

329. The composition of any one of the preceding Embodiments, wherein the composition is capable of selectively increasing the level of skipping of a deleterious exon in an USH2A gene transcript that is associated with a condition, disorder or disease.

330. The oligonucleotide of Embodiment 329, wherein the condition, disorder or disease is Usher Syndrome.

331. The oligonucleotide of Embodiment 329, wherein the condition, disorder or disease is Usher Syndrome type 2A.

332. The oligonucleotide of any one of Embodiment 329-331, wherein the USH2A gene associated with Usher Syndrome comprises at least one disease-associated mutation.

333. The composition of any one of the preceding Embodiments, wherein the composition is capable of selectively increasing the level of skipping of human exon 13 over skipping of human exon 12 or skipping of human exons 12 and 13.

334. The composition of any one of Embodiments 329-333, wherein the selectivity is at least 2, 3, 4, 5, 6, 7, 8, 9, 10, 15, 20, 25, 30, 35, 40, 50, 60, 70, 80, 90 or 100 fold.

335. The composition of any one of Embodiments 329-334, wherein the selectivity is at least 2 fold.

336. A method for preventing, treating or ameliorating an USH2A-related condition, disorder or disease and/or preventing, slowing the onset, development and/or progress, and/or treating an USH2A-related condition, disorder or disease in a subject susceptible thereto or suffering therefrom, comprising administering to the subject a therapeutically effective amount of an oligonucleotide or a pharmaceutical composition of any one of the preceding Embodiments.

337. The method of Embodiment 336, wherein the condition, disorder or disease is Usher Syndrome.

338. The method of Embodiment 336, wherein the condition, disorder or disease is Usher Syndrome type 2A.

339. A method for increasing the level of skipping of a deleterious exon in an USH2A gene transcript or its gene product in a cell, comprising contacting the cell with an oligonucleotide or composition of any one of the preceding Embodiments.

340. A method for preventing, delaying onset or progression of, treating or ameliorating an USH2A-related condition, disorder or disease in a subject susceptible thereto or suffering therefrom, wherein the genome of the subject comprises a deleterious mutation in exon 13 of an USH2A allele, comprising administering to the subject an effective amount of an oligonucleotide or composition of any one of the preceding Embodiments.

341. The method of Embodiment 340, wherein the condition, disorder or disease is Usher Syndrome Type 2A, atypical Usher syndrome, or nonsyndromic retinitis pigmentosa.

342. The method of Embodiment 340, wherein the condition, disorder or disease is Usher Syndrome Type 2A.

343. A method for increasing the level of skipping of a deleterious exon in an USH2A gene transcript or its gene product in a cell, comprising contacting the cell with an oligonucleotide or composition of any one of the preceding Embodiments.

344. A method for skipping a deleterious exon 13 in an allele of USH2A in a subject, comprising administering to the subject an effective amount of an oligonucleotide or composition of any one of the preceding Embodiments.

345. The method of Embodiment 344, wherein when the oligonucleotide, or the oligonucleotide composition, is contacted with a system comprising transcripts of both the target nucleic acid sequence and a similar nucleic acid sequences, transcripts of the target nucleic acid sequence are suppressed at a greater level than a level of suppression observed for a similar nucleic acid sequence.

346. The method of Embodiment 345, wherein suppression of the transcripts of the target nucleic acid sequence is 1.5, 2, 2.5, 3, 4, 5, 6, 7, 8, 9, or 10 fold of suppression observed for a similar nucleic acid sequence.

347. The method of any one of Embodiments 344-346, wherein the transcripts of the target nucleic acid sequence are associated with a condition, disorder or disease.

348. The method of any one of Embodiments 344-347, wherein the condition, disorder or disease is Usher Syndrome.

349. The method of any one of Embodiments 344-348, wherein transcripts of a similar nucleic acid sequence is not, or is less, associated with the condition, disorder or disease.

350. A method for increasing skipping of a deleterious exon in a transcript from a target USH2A sequence for which a plurality of alleles exist within a population, each of which contains a specific characteristic sequence element that defines the allele relative to other alleles of the same target sequence, the method comprising steps of:
contacting a sample comprising transcripts of the target nucleic acid sequence with an oligonucleotide or an oligonucleotide composition comprising a plurality of oligonucleotides sharing a common base sequence,
wherein the level of skipping of a deleterious exon in the USH2A gene transcript is increased.

351. The method of Embodiment 350, wherein when the oligonucleotide, or the oligonucleotide composition, is contacted with a system comprising transcripts of both the target allele and another allele of the same nucleic acid sequence, transcripts of the particular allele are suppressed at a greater level than a level of suppression observed for another allele of the same nucleic acid sequence.

352. The method of Embodiment 351, wherein suppression of the transcripts of the particularly allele is 1.5, 2, 2.5, 3, 4, 5, 6, 7, 8, 9, or 10 fold of suppression observed for another allele.

353. The method of any one of Embodiments 350-352, wherein the transcripts of the particular allele are associated with a condition, disorder or disease.

354. The method of any one of Embodiments 350-353, wherein the condition, disorder or disease is Usher Syndrome.

355. The method of any one of Embodiments 350-353, wherein the condition, disorder or disease is Usher Syndrome type 2A.

356. The method of any one of Embodiments 350-355, wherein transcripts of another allele is not, or is less, associated with the condition, disorder or disease.

357. The method of any one of Embodiments 344-356, wherein the characteristic sequence element comprises a mutation.

358. The method of any one of Embodiments 344-357, wherein the characteristic sequence element comprises a mutation in an exon.

359. The method of any one of Embodiments 344-358, wherein the characteristic sequence element comprises a mutation in exon 13.

360. The method of any one of Embodiments 344-359, wherein the oligonucleotide or the oligonucleotide composition is of any one of Embodiments 1-336.

361. A method for producing or increasing level of an exon 13-skipped USH2A protein in a system, comprising administering to the system an oligonucleotide or composition of any one of the preceding Embodiments.

362. The method of Embodiment 361, wherein the system is a cell.

363. The method of Embodiment 361, wherein the system is a tissue.

364. The method of Embodiment 361, wherein the system is an organ.

365. The method of any one of Embodiments 361-364, wherein the system is or comprises a cell or tissue of an eye, or is an eye.

366. The method of any one of Embodiments 361-364, wherein the system is or comprises a cell or tissue of a retina.

367. The method of Embodiment 361, wherein the system is a human.

368. A compound, oligonucleotide, composition, or method described in the specification.

While various embodiments have been described and illustrated herein, those of ordinary skill in the art will readily envision a variety of other means and/or structures for performing the functions and/or obtaining the results and/or one or more of the advantages described in the present disclosure, and each of such variations and/or modifications is deemed to be included. More generally, those skilled in the art will readily appreciate that all parameters, dimensions, materials, and configurations described herein are meant to be example and that the actual parameters, dimensions, materials, and/or configurations may depend upon the specific application or applications for which the teachings of the present disclosure is/are used. Those skilled in the art will recognize, or be able to ascertain using no more than routine experimentation, many equivalents to the embodiments of the present disclosure. It is, therefore, to be understood that the foregoing embodiments are presented by way of example only and that, within the scope of the appended claims and equivalents thereto, claimed technologies may be practiced otherwise than as specifically described and claimed. In addition, any combination of two or more features, systems, articles, materials, kits, and/or methods, if such features, systems, articles, materials, kits, and/or methods are not mutually inconsistent, is included within the scope of the present disclosure.

1.-26. (canceled)

27. A chirally controlled oligonucleotide composition comprising a plurality of oligonucleotides, wherein the oligonucleotides share:
1) a common base sequence,
2) a common pattern of backbone linkages, and
3) a common pattern of backbone chiral centers,
wherein the composition is enriched, relative to a substantially racemic preparation of oligonucleotides sharing the common base sequence and pattern of backbone linkages, for oligonucleotides of the plurality, and
wherein:
oligonucleotides of the plurality each independently comprise a phosphorothioate internucleotidic linkage,
oligonucleotides of the plurality each independently comprise a non-negatively charged internucleotidic linkages, and
the base sequence comprises at least 15 contiguous nucleobases of a base sequence that is at least 75% identical or complementary to a base sequence of an USH2A gene or a transcript thereof.

28. (canceled)

29. The composition of claim 27, wherein oligonucleotides of the plurality share the same linkage phosphorus stereochemistry at 5 or more chiral internucleotidic linkages.

30. (canceled)

31. The composition of claim 29, wherein about 30-100% of all oligonucleotides within the composition that share the common base sequence are oligonucleotides of the plurality.

32.-33. (canceled)

34. A method for preventing, treating or ameliorating an USH2A-related condition, disorder or disease and/or preventing, slowing the onset, development and/or progress, and/or treating an USH2A-related condition, disorder or disease in a subject susceptible thereto or suffering therefrom, comprising administering to the subject a therapeutically effective amount of a composition of claim 27.

35. The method of claim 34, wherein the condition, disorder or disease is Usher Syndrome.

36. (canceled)

37. A method for increasing the level of skipping of a deleterious exon in an USH2A gene transcript or its gene product in a cell, comprising contacting the cell with a composition of claim 27.

38.-44. (canceled)

45. The composition of claim 27, wherein oligonucleotides of the plurality consists of or comprises a structure of 5′-a first region-a second region-a third region-3′, wherein each of the regions independently comprises one or more nucleosides.

46. The composition of claim 45, wherein the first region comprises 5 or more nucleosides.

47. The composition of claim 46, wherein the first region comprises two or more 2′-F modified sugars.

48. The composition of claim 47, wherein the second region comprises two or more nucleosides.

49. The composition of claim 48, wherein the second region comprises one or more 2′-F modified sugars and one or more 2′-OR modified sugars, wherein R is optionally substituted C₁₋₆ aliphatic.

50. The composition of claim 49, wherein the third region comprises 5 or more nucleosides.

51. The composition of claim 50, wherein the third region comprises two or more 2′-F modified sugars.

52. The composition of claim 51, wherein the first region comprises one or more Sp phosphorothioate internucleotidic linkages.

53. The composition of claim 52, wherein the first region comprises one or more non-negatively charged internucleotidic linkages.

54. The composition of claim 53, wherein the second region comprises one or more Sp phosphorothioate internucleotidic linkages.

55. The composition of claim 54, wherein the second region comprises one or more non-negatively charged internucleotidic linkages.

56. The composition of claim 55, wherein the third region comprises one or more Sp phosphorothioate internucleotidic linkages.

57. The composition of claim 56, wherein the third region comprises one or more non-negatively charged internucleotidic linkages.

58. The composition of claim 57, wherein oligonucleotides of the plurality are capable of increasing the level of skipping of a deleterious exon in an USH2A gene transcript or a gene product thereof, wherein the deleterious exon is associated with Usher Syndrome.


SEQUENCE LISTING (342 sequences)

SEQ ID NO: 1 - RNA, 20 nt, Artificial Sequence
  Synthetic
  misc_feature (1)..(20): each U can be independently substituted with T
aagcccuaaagauaaaauau

SEQ ID NO: 2 - RNA, 20 nt, Artificial Sequence
  Synthetic
  misc_feature (1)..(20): each U can be independently substituted with T
aauacauuucuuucuuaccu

SEQ ID NO: 3 - RNA, 20 nt, Artificial Sequence
  Synthetic
  misc_feature (1)..(20): each U can be independently substituted with T
acauccaacaucauuaaagc

SEQ ID NO: 4 - RNA, 21 nt, Artificial Sequence
  Synthetic
  misc_feature (1)..(21): each U can be independently substituted with T
agcuucggagaaauuuaaauc

SEQ ID NO: 5 - RNA, 21 nt, Artificial Sequence
  Synthetic
  misc_feature (1)..(21): each U can be independently substituted with T
agcuucggagaaauuuaaauc

SEQ ID NO: 6 - RNA, 20 nt, Artificial Sequence
  Synthetic
  misc_feature (1)..(20): each U can be independently substituted with T
aggauugcagaauuuguuca

SEQ ID NO: 7 - RNA, 20 nt, Artificial Sequence
  Synthetic
  misc_feature (1)..(20): each U can be independently substituted with T
aggauugcagaauuuguuca

SEQ ID NO: 8 - RNA, 20 nt, Artificial Sequence
  Synthetic
  misc_feature (1)..(20): each U can be independently substituted with T
auccaaaauugcaaugauca

SEQ ID NO: 9 - RNA, 20 nt, Artificial Sequence
  Synthetic
  misc_feature (1)..(20): each U can be independently substituted with T
auuucuuucuuaccugguug

SEQ ID NO: 10 - RNA, 20 nt, Artificial Sequence
  Synthetic
  misc_feature (1)..(20): each U can be independently substituted with T
caacaucauuaaagcuucgg

SEQ ID NO: 11 - RNA, 20 nt, Artificial Sequence
  Synthetic
  misc_feature (1)..(20): each U can be independently substituted with T
caccuaagcccuaaagauaa

SEQ ID NO: 12 - RNA, 20 nt, Artificial Sequence
  Synthetic
  misc_feature (1)..(20): each U can be independently substituted with T
gaggauugcagaauuuguuc

SEQ ID NO: 13 - RNA, 20 nt, Artificial Sequence
  Synthetic
  misc_feature (1)..(20): each U can be independently substituted with T
gaucacaccuaagcccuaaa

SEQ ID NO: 14 - RNA, 20 nt, Artificial Sequence
  Synthetic
  misc_feature (1)..(20): each U can be independently substituted with T
gauugcagaauuuguucacu

SEQ ID NO: 15 - RNA, 20 nt, Artificial Sequence
  Synthetic
  misc_feature (1)..(20): each U can be independently substituted with T
gcaaugaucacaccuaagcc

SEQ ID NO: 16 - RNA, 20 nt, Artificial Sequence
  Synthetic
  misc_feature (1)..(20): each U can be independently substituted with T
gcuucggagaaauuuaaauc

SEQ ID NO: 17 - RNA, 20 nt, Artificial Sequence
  Synthetic
  misc_feature (1)..(20): each U can be independently substituted with T
ggaaucacacucacacaucu

SEQ ID NO: 18 - RNA, 20 nt, Artificial Sequence
  Synthetic
  misc_feature (1)..(20): each U can be independently substituted with T
ggauugcagaauuuguucac

SEQ ID NO: 19 - RNA, 19 nt, Artificial Sequence
  Synthetic
  misc_feature (1)..(19): each U can be independently substituted with T
ggauugcagaauuuguuca

SEQ ID NO: 20 - RNA, 20 nt, Artificial Sequence
  Synthetic
  misc_feature (1)..(20): each U can be independently substituted with T
uaccugguugacacugauua

SEQ ID NO: 21 - RNA, 20 nt, Artificial Sequence
  Synthetic
  misc_feature (1)..(20): each U can be independently substituted with T
uaccugguugacacugauua

SEQ ID NO: 22 - RNA, 20 nt, Artificial Sequence
  Synthetic
  misc_feature (1)..(20): each U can be independently substituted with T
ucuuuuuugcacucacacug

SEQ ID NO: 23 - RNA, 20 nt, Artificial Sequence
  Synthetic
  misc_feature (1)..(20): each U can be independently substituted with T
ugaggauugcagaauuuguu

SEQ ID NO: 24 - RNA, 20 nt, Artificial Sequence
  Synthetic
  misc_feature (1)..(20): each U can be independently substituted with T
ugaggauugcagaauuuguu

SEQ ID NO: 25 - RNA, 20 nt, Artificial Sequence
  Synthetic
  misc_feature (1)..(20): each U can be independently substituted with T
ugcagaauuuguucacugag

SEQ ID NO: 26 - RNA, 20 nt, Artificial Sequence
  Synthetic
  misc_feature (1)..(20): each U can be independently substituted with T
uugcagaauuuguucacuga

SEQ ID NO: 27 - RNA, 20 nt, Artificial Sequence
  Synthetic
  misc_feature (1)..(20): each U can be independently substituted with T
uuucuuaccugguugacacu

SEQ ID NO: 28 - RNA, 19 nt, Artificial Sequence
  Synthetic
gugcuaguagccaaccccc

SEQ ID NO: 29 - DNA, 19 bp, Artificial Sequence
  Synthetic
gggggttggctactagcac

SEQ ID NO: 30 - RNA, 21 nt, Artificial Sequence
  Synthetic
agcuucggagaaauuuaaauc

SEQ ID NO: 31 - DNA, 21 bp, Artificial Sequence
  Synthetic
agcttcggagaaatttaaatc

SEQ ID NO: 32 - DNA, 21 bp, Artificial Sequence
  Synthetic
agcttcggagaaauuuaaatc

SEQ ID NO: 33 - RNA, 21 nt, Artificial Sequence
  Synthetic
agcuucggagaaauuuaaauc

SEQ ID NO: 34 - RNA, 21 nt, Artificial Sequence
  Synthetic
agcuucggagaaauuuaaauc

SEQ ID NO: 35 - RNA, 20 nt, Artificial Sequence
  Synthetic
cuaaagauaaaauauauuua

SEQ ID NO: 36 - RNA, 20 nt, Artificial Sequence
  Synthetic
aagcccuaaagauaaaauau

SEQ ID NO: 37 - RNA, 20 nt, Artificial Sequence
  Synthetic
caccuaagcccuaaagauaa

SEQ ID NO: 38 - RNA, 20 nt, Artificial Sequence
  Synthetic
gaucacaccuaagcccuaaa

SEQ ID NO: 39 - RNA, 20 nt, Artificial Sequence
  Synthetic
gcaaugaucacaccuaagcc

SEQ ID NO: 40 - RNA, 20 nt, Artificial Sequence
  Synthetic
aaauugcaaugaucacaccu

SEQ ID NO: 41 - RNA, 20 nt, Artificial Sequence
  Synthetic
auccaaaauugcaaugauca

SEQ ID NO: 42 - RNA, 20 nt, Artificial Sequence
  Synthetic
uuuaaauccaaaauugcaau

SEQ ID NO: 43 - RNA, 20 nt, Artificial Sequence
  Synthetic
agaaauuuaaauccaaaauu

SEQ ID NO: 44 - RNA, 20 nt, Artificial Sequence
  Synthetic
uucggagaaauuuaaaucca

SEQ ID NO: 45 - RNA, 20 nt, Artificial Sequence
  Synthetic
aaagcuucggagaaauuuaa

SEQ ID NO: 46 - RNA, 20 nt, Artificial Sequence
  Synthetic
ucauuaaagcuucggagaaa

SEQ ID NO: 47 - RNA, 20 nt, Artificial Sequence
  Synthetic
caacaucauuaaagcuucgg

SEQ ID NO: 48 - RNA, 20 nt, Artificial Sequence
  Synthetic
acauccaacaucauuaaagc

SEQ ID NO: 49 - RNA, 20 nt, Artificial Sequence
  Synthetic
ggcucacauccaacaucauu

SEQ ID NO: 50 - RNA, 20 nt, Artificial Sequence
  Synthetic
ggcagggcucacauccaaca

SEQ ID NO: 51 - RNA, 20 nt, Artificial Sequence
  Synthetic
acacuggcagggcucacauc

SEQ ID NO: 52 - RNA, 20 nt, Artificial Sequence
  Synthetic
agguuacacuggcagggcuc

SEQ ID NO: 53 - RNA, 20 nt, Artificial Sequence
  Synthetic
cauggagguuacacuggcag

SEQ ID NO: 54 - RNA, 20 nt, Artificial Sequence
  Synthetic
ugagccauggagguuacacu

SEQ ID NO: 55 - RNA, 20 nt, Artificial Sequence
  Synthetic
uucacugagccauggagguu

SEQ ID NO: 56 - RNA, 20 nt, Artificial Sequence
  Synthetic
auuuguucacugagccaugg

SEQ ID NO: 57 - RNA, 20 nt, Artificial Sequence
  Synthetic
gcagaauuuguucacugagc

SEQ ID NO: 58 - RNA, 20 nt, Artificial Sequence
  Synthetic
ggauugcagaauuuguucac

SEQ ID NO: 59 - RNA, 20 nt, Artificial Sequence
  Synthetic
agugaggauugcagaauuug

SEQ ID NO: 60 - RNA, 20 nt, Artificial Sequence
  Synthetic
cccagagugaggauugcaga

SEQ ID NO: 61 - RNA, 20 nt, Artificial Sequence
  Synthetic
cacugcccagagugaggauu

SEQ ID NO: 62 - RNA, 20 nt, Artificial Sequence
  Synthetic
acucacacugcccagaguga

SEQ ID NO: 63 - RNA, 20 nt, Artificial Sequence
  Synthetic
uuugcacucacacugcccag

SEQ ID NO: 64 - RNA, 20 nt, Artificial Sequence
  Synthetic
ucuuuuuugcacucacacug

SEQ ID NO: 65 - RNA, 20 nt, Artificial Sequence
  Synthetic
uggcuucuuuuuugcacuca

SEQ ID NO: 66 - RNA, 20 nt, Artificial Sequence
  Synthetic
uccuuuggcuucuuuuuugc

SEQ ID NO: 67 - RNA, 20 nt, Artificial Sequence
  Synthetic
ugaaguccuuuggcuucuuu

SEQ ID NO: 68 - RNA, 20 nt, Artificial Sequence
  Synthetic
cacacugaaguccuuuggcu

SEQ ID NO: 69 - RNA, 20 nt, Artificial Sequence
  Synthetic
ggugucacacugaaguccuu

SEQ ID NO: 70 - RNA, 20 nt, Artificial Sequence
  Synthetic
cugcaggugucacacugaag

SEQ ID NO: 71 - RNA, 20 nt, Artificial Sequence
  Synthetic
uuucucugcaggugucacac

SEQ ID NO: 72 - RNA, 20 nt, Artificial Sequence
  Synthetic
aaaguuuucucugcaggugu

SEQ ID NO: 73 - RNA, 20 nt, Artificial Sequence
  Synthetic
ccauaaaaguuuucucugca

SEQ ID NO: 74 - RNA, 20 nt, Artificial Sequence
  Synthetic
cuaacccauaaaaguuuucu

SEQ ID NO: 75 - RNA, 20 nt, Artificial Sequence
  Synthetic
gacaucuaacccauaaaagu

SEQ ID NO: 76 - RNA, 20 nt, Artificial Sequence
  Synthetic
uuggugacaucuaacccaua

SEQ ID NO: 77 - RNA, 20 nt, Artificial Sequence
  Synthetic
uacaauuggugacaucuaac

SEQ ID NO: 78 - RNA, 20 nt, Artificial Sequence
  Synthetic
ggccuuacaauuggugacau

SEQ ID NO: 79 - RNA, 20 nt, Artificial Sequence
  Synthetic
ucacaggccuuacaauuggu

SEQ ID NO: 80 - RNA, 20 nt, Artificial Sequence
  Synthetic
cacagucacaggccuuacaa

SEQ ID NO: 81 - RNA, 20 nt, Artificial Sequence
  Synthetic
ugugucacagucacaggccu

SEQ ID NO: 82 - RNA, 20 nt, Artificial Sequence
  Synthetic
ccagcugugucacagucaca

SEQ ID NO: 83 - RNA, 20 nt, Artificial Sequence
  Synthetic
gggauccagcugugucacag

SEQ ID NO: 84 - RNA, 20 nt, Artificial Sequence
  Synthetic
agggagggauccagcugugu

SEQ ID NO: 85 - RNA, 20 nt, Artificial Sequence
  Synthetic
gucccagggagggauccagc

SEQ ID NO: 86 - RNA, 20 nt, Artificial Sequence
  Synthetic
agacagucccagggagggau

SEQ ID NO: 87 - RNA, 20 nt, Artificial Sequence
  Synthetic
auuacagacagucccaggga

SEQ ID NO: 88 - RNA, 20 nt, Artificial Sequence
  Synthetic
uuagcauuacagacaguccc

SEQ ID NO: 89 - RNA, 20 nt, Artificial Sequence
  Synthetic
cugucuuagcauuacagaca

SEQ ID NO: 90 - RNA, 20 nt, Artificial Sequence
  Synthetic
cugcccugucuuagcauuac

SEQ ID NO: 91 - RNA, 20 nt, Artificial Sequence
  Synthetic
augcacugcccugucuuagc

SEQ ID NO: 92 - RNA, 20 nt, Artificial Sequence
  Synthetic
ugcagaugcacugcccuguc

SEQ ID NO: 93 - RNA, 20 nt, Artificial Sequence
  Synthetic
gggcuugcagaugcacugcc

SEQ ID NO: 94 - RNA, 20 nt, Artificial Sequence
  Synthetic
acauugggcuugcagaugca

SEQ ID NO: 95 - RNA, 20 nt, Artificial Sequence
  Synthetic
cuucaacauugggcuugcag

SEQ ID NO: 96 - RNA, 20 nt, Artificial Sequence
  Synthetic
ucucccuucaacauugggcu

SEQ ID NO: 97 - RNA, 20 nt, Artificial Sequence
  Synthetic
cacugucucccuucaacauu

SEQ ID NO: 98 - RNA, 20 nt, Artificial Sequence
  Synthetic
uauugcacugucucccuuca

SEQ ID NO: 99 - RNA, 20 nt, Artificial Sequence
  Synthetic
acauuuauugcacugucucc

SEQ ID NO: 100 - RNA, 20 nt, Artificial Sequence
  Synthetic
uccaaacauuuauugcacug

SEQ ID NO: 101 - RNA, 20 nt, Artificial Sequence
  Synthetic
uucccuccaaacauuuauug

SEQ ID NO: 102 - RNA, 20 nt, Artificial Sequence
  Synthetic
gaaguuucccuccaaacauu

SEQ ID NO: 103 - RNA, 20 nt, Artificial Sequence
  Synthetic
agguagaaguuucccuccaa

SEQ ID NO: 104 - RNA, 20 nt, Artificial Sequence
  Synthetic
gccguagguagaaguuuccc

SEQ ID NO: 105 - RNA, 20 nt, Artificial Sequence
  Synthetic
auuuugccguagguagaagu

SEQ ID NO: 106 - RNA, 20 nt, Artificial Sequence
  Synthetic
gaauuauuuugccguaggua

SEQ ID NO: 107 - RNA, 20 nt, Artificial Sequence
  Synthetic
ggaaagaauuauuuugccgu

SEQ ID NO: 108 - RNA, 20 nt, Artificial Sequence
  Synthetic
acagaggaaagaauuauuuu

SEQ ID NO: 109 - RNA, 20 nt, Artificial Sequence
  Synthetic
ggcagacagaggaaagaauu

SEQ ID NO: 110 - RNA, 20 nt, Artificial Sequence
  Synthetic
ugcaaggcagacagaggaaa

SEQ ID NO: 111 - RNA, 20 nt, Artificial Sequence
  Synthetic
acaguugcaaggcagacaga

SEQ ID NO: 112 - RNA, 20 nt, Artificial Sequence
  Synthetic
uuaucacaguugcaaggcag

SEQ ID NO: 113 - RNA, 20 nt, Artificial Sequence
  Synthetic
cagucuuaucacaguugcaa

SEQ ID NO: 114 - RNA, 20 nt, Artificial Sequence
  Synthetic
ugucccagucuuaucacagu

SEQ ID NO: 115 - RNA, 20 nt, Artificial Sequence
  Synthetic
uuuauugucccagucuuauc

SEQ ID NO: 116 - RNA, 20 nt, Artificial Sequence
  Synthetic
agccauuuauugucccaguc

SEQ ID NO: 117 - RNA, 20 nt, Artificial Sequence
  Synthetic
cagagagccauuuauugucc

SEQ ID NO: 118 - RNA, 20 nt, Artificial Sequence
  Synthetic
cacagcagagagccauuuau

SEQ ID NO: 119 - RNA, 20 nt, Artificial Sequence
  Synthetic
uguuacacagcagagagcca

SEQ ID NO: 120 - RNA, 20 nt, Artificial Sequence
  Synthetic
ugauuuguuacacagcagag

SEQ ID NO: 121 - RNA, 20 nt, Artificial Sequence
  Synthetic
ccuguugauuuguuacacag

SEQ ID NO: 122 - RNA, 20 nt, Artificial Sequence
  Synthetic
auuguccuguugauuuguua

SEQ ID NO: 123 - RNA, 20 nt, Artificial Sequence
  Synthetic
aggacauuguccuguugauu

SEQ ID NO: 124 - RNA, 20 nt, Artificial Sequence
  Synthetic
uugcaaggacauuguccugu

SEQ ID NO: 125 - RNA, 20 nt, Artificial Sequence
  Synthetic
cuaauuugcaaggacauugu

SEQ ID NO: 126 - RNA, 20 nt, Artificial Sequence
  Synthetic
uaccccuaauuugcaaggac

SEQ ID NO: 127 - RNA, 20 nt, Artificial Sequence
  Synthetic
ccuguuaccccuaauuugca

SEQ ID NO: 128 - RNA, 20 nt, Artificial Sequence
  Synthetic
gaagaccuguuaccccuaau

SEQ ID NO: 129 - RNA, 20 nt, Artificial Sequence
  Synthetic
acagcgaagaccuguuaccc

SEQ ID NO: 130 - RNA, 20 nt, Artificial Sequence
  Synthetic
ugauuacagcgaagaccugu

SEQ ID NO: 131 - RNA, 20 nt, Artificial Sequence
  Synthetic
cacacugauuacagcgaaga

SEQ ID NO: 132 - RNA, 20 nt, Artificial Sequence
  Synthetic
aggcucacacugauuacagc

SEQ ID NO: 133 - RNA, 20 nt, Artificial Sequence
  Synthetic
cugugaggcucacacugauu

SEQ ID NO: 134 - RNA, 20 nt, Artificial Sequence
  Synthetic
uguaccugugaggcucacac

SEQ ID NO: 135 - RNA, 20 nt, Artificial Sequence
  Synthetic
caaauuguaccugugaggcu

SEQ ID NO: 136 - RNA, 20 nt, Artificial Sequence
  Synthetic
auggucaaauuguaccugug

SEQ ID NO: 137 - RNA, 20 nt, Artificial Sequence
  Synthetic
ugucaauggucaaauuguac

SEQ ID NO: 138 - RNA, 20 nt, Artificial Sequence
  Synthetic
aaaauugucaauggucaaau

SEQ ID NO: 139 - RNA, 20 nt, Artificial Sequence
  Synthetic
uguugaaaauugucaauggu

SEQ ID NO: 140 - RNA, 20 nt, Artificial Sequence
  Synthetic
ggcaguguugaaaauuguca

SEQ ID NO: 141 - RNA, 20 nt, Artificial Sequence
  Synthetic
caucuggcaguguugaaaau

SEQ ID NO: 142 - RNA, 20 nt, Artificial Sequence
  Synthetic
ucacacaucuggcaguguug

SEQ ID NO: 143 - RNA, 20 nt, Artificial Sequence
  Synthetic
cacacucacacaucuggcag

SEQ ID NO: 144 - RNA, 20 nt, Artificial Sequence
  Synthetic
ggaaucacacucacacaucu

SEQ ID NO: 145 - RNA, 20 nt, Artificial Sequence
  Synthetic
cccaaggaaucacacucaca

SEQ ID NO: 146 - RNA, 20 nt, Artificial Sequence
  Synthetic
auguccccaaggaaucacac

SEQ ID NO: 147 - RNA, 20 nt, Artificial Sequence
  Synthetic
agguaauguccccaaggaau

SEQ ID NO: 148 - RNA, 20 nt, Artificial Sequence
  Synthetic
gucccagguaauguccccaa

SEQ ID NO: 149 - RNA, 20 nt, Artificial Sequence
  Synthetic
aaauggucccagguaauguc

SEQ ID NO: 150 - RNA, 20 nt, Artificial Sequence
  Synthetic
gucacaaauggucccaggua

SEQ ID NO: 151 - RNA, 20 nt, Artificial Sequence
  Synthetic
auugggucacaaaugguccc

SEQ ID NO: 152 - RNA, 20 nt, Artificial Sequence
  Synthetic
cacugauugggucacaaaug

SEQ ID NO: 153 - RNA, 20 nt, Artificial Sequence
  Synthetic
cuggccacugauugggucac

SEQ ID NO: 154 - RNA, 20 nt, Artificial Sequence
  Synthetic
aggcacuggccacugauugg

SEQ ID NO: 155 - RNA, 20 nt, Artificial Sequence
  Synthetic
cacacaggcacuggccacug

SEQ ID NO: 156 - RNA, 20 nt, Artificial Sequence
  Synthetic
aggcacacacaggcacuggc

SEQ ID NO: 157 - RNA, 20 nt, Artificial Sequence
  Synthetic
cgauuaggcacacacaggca

SEQ ID NO: 158 - RNA, 20 nt, Artificial Sequence
  Synthetic
cuugacgauuaggcacacac

SEQ ID NO: 159 - RNA, 20 nt, Artificial Sequence
  Synthetic
ucuuccuugacgauuaggca

SEQ ID NO: 160 - RNA, 20 nt, Artificial Sequence
  Synthetic
caccuucuuccuugacgauu

SEQ ID NO: 161 - RNA, 20 nt, Artificial Sequence
  Synthetic
gauuacaccuucuuccuuga

SEQ ID NO: 162 - RNA, 20 nt, Artificial Sequence
  Synthetic
acacugauuacaccuucuuc

SEQ ID NO: 163 - RNA, 20 nt, Artificial Sequence
  Synthetic
gguugacacugauuacaccu

SEQ ID NO: 164 - RNA, 20 nt, Artificial Sequence
  Synthetic
uaccugguugacacugauua

SEQ ID NO: 165 - RNA, 20 nt, Artificial Sequence
  Synthetic
uuucuuaccugguugacacu

SEQ ID NO: 166 - RNA, 20 nt, Artificial Sequence
  Synthetic
auuucuuucuuaccugguug

SEQ ID NO: 167 - RNA, 20 nt, Artificial Sequence
  Synthetic
aauacauuucuuucuuaccu

SEQ ID NO: 168 - RNA, 20 nt, Artificial Sequence
  Synthetic
guaauacauuucuuucuuac

SEQ ID NO: 169 - RNA, 21 nt, Artificial Sequence
  Synthetic
agcuucggagaaauuuaaauc

SEQ ID NO: 170 - RNA, 20 nt, Artificial Sequence
  Synthetic
agcuucggagaaauuuaaau

SEQ ID NO: 171 - RNA, 20 nt, Artificial Sequence
  Synthetic
gcuucggagaaauuuaaauc

SEQ ID NO: 172 - RNA, 21 nt, Artificial Sequence
  Synthetic
agcuucggagaaauuuaaauc

SEQ ID NO: 173 - RNA, 20 nt, Artificial Sequence
  Synthetic
agcuucggagaaauuuaaau

SEQ ID NO: 174 - RNA, 20 nt, Artificial Sequence
  Synthetic
gcuucggagaaauuuaaauc

SEQ ID NO: 175 - RNA, 21 nt, Artificial Sequence
  Synthetic
agcuucggagaaauuuaaauc

SEQ ID NO: 176 - RNA, 20 nt, Artificial Sequence
  Synthetic
agcuucggagaaauuuaaau

SEQ ID NO: 177 - RNA, 20 nt, Artificial Sequence
  Synthetic
gcuucggagaaauuuaaauc

SEQ ID NO: 178 - RNA, 21 nt, Artificial Sequence
  Synthetic
agcuucggagaaauuuaaauc

SEQ ID NO: 179 - RNA, 20 nt, Artificial Sequence
  Synthetic
agcuucggagaaauuuaaau

SEQ ID NO: 180 - RNA, 20 nt, Artificial Sequence
  Synthetic
gcuucggagaaauuuaaauc

SEQ ID NO: 181 - RNA, 20 nt, Artificial Sequence
  Synthetic
gugaggauugcagaauuugu

SEQ ID NO: 182 - RNA, 20 nt, Artificial Sequence
  Synthetic
ugaggauugcagaauuuguu

SEQ ID NO: 183 - RNA, 20 nt, Artificial Sequence
  Synthetic
gaggauugcagaauuuguuc

SEQ ID NO: 184 - RNA, 20 nt, Artificial Sequence
  Synthetic
aggauugcagaauuuguuca

SEQ ID NO: 185 - RNA, 20 nt, Artificial Sequence
  Synthetic
gauugcagaauuuguucacu

SEQ ID NO: 186 - RNA, 20 nt, Artificial Sequence
  Synthetic
auugcagaauuuguucacug

SEQ ID NO: 187 - RNA, 20 nt, Artificial Sequence
  Synthetic
uugcagaauuuguucacuga

SEQ ID NO: 188 - RNA, 20 nt, Artificial Sequence
  Synthetic
ugcagaauuuguucacugag

SEQ ID NO: 189 - RNA, 20 nt, Artificial Sequence
  Synthetic
uucuuaccugguugacacug

SEQ ID NO: 190 - RNA, 20 nt, Artificial Sequence
  Synthetic
ucuuaccugguugacacuga

SEQ ID NO: 191 - RNA, 20 nt, Artificial Sequence
  Synthetic
cuuaccugguugacacugau

SEQ ID NO: 192 - RNA, 20 nt, Artificial Sequence
  Synthetic
uuaccugguugacacugauu

SEQ ID NO: 193 - RNA, 20 nt, Artificial Sequence
  Synthetic
accugguugacacugauuac

SEQ ID NO: 194 - RNA, 20 nt, Artificial Sequence
  Synthetic
ccugguugacacugauuaca

SEQ ID NO: 195 - RNA, 20 nt, Artificial Sequence
  Synthetic
cugguugacacugauuacac

SEQ ID NO: 196 - RNA, 20 nt, Artificial Sequence
  Synthetic
ugguugacacugauuacacc

SEQ ID NO: 197 - RNA, 20 nt, Artificial Sequence
  Synthetic
gugaggauugcagaauuugu

SEQ ID NO: 198 - RNA, 20 nt, Artificial Sequence
  Synthetic
ugaggauugcagaauuuguu

SEQ ID NO: 199 - RNA, 20 nt, Artificial Sequence
  Synthetic
gaggauugcagaauuuguuc

SEQ ID NO: 200 - RNA, 20 nt, Artificial Sequence
  Synthetic
aggauugcagaauuuguuca

SEQ ID NO: 201 - RNA, 20 nt, Artificial Sequence
  Synthetic
ggauugcagaauuuguucac

SEQ ID NO: 202 - RNA, 20 nt, Artificial Sequence
  Synthetic
gauugcagaauuuguucacu

SEQ ID NO: 203 - RNA, 20 nt, Artificial Sequence
  Synthetic
auugcagaauuuguucacug

SEQ ID NO: 204 - RNA, 20 nt, Artificial Sequence
  Synthetic
uugcagaauuuguucacuga

SEQ ID NO: 205 - RNA, 20 nt, Artificial Sequence
  Synthetic
ugcagaauuuguucacugag

SEQ ID NO: 206 - RNA, 20 nt, Artificial Sequence
  Synthetic
gugaggauugcagaauuugu

SEQ ID NO: 207 - RNA, 20 nt, Artificial Sequence
  Synthetic
ugaggauugcagaauuuguu

SEQ ID NO: 208 - RNA, 20 nt, Artificial Sequence
  Synthetic
gaggauugcagaauuuguuc

SEQ ID NO: 209 - RNA, 20 nt, Artificial Sequence
  Synthetic
aggauugcagaauuuguuca

SEQ ID NO: 210 - RNA, 20 nt, Artificial Sequence
  Synthetic
ggauugcagaauuuguucac

SEQ ID NO: 211 - RNA, 20 nt, Artificial Sequence
  Synthetic
gauugcagaauuuguucacu

SEQ ID NO: 212 - RNA, 20 nt, Artificial Sequence
  Synthetic
auugcagaauuuguucacug

SEQ ID NO: 213 - RNA, 20 nt, Artificial Sequence
  Synthetic
uugcagaauuuguucacuga

SEQ ID NO: 214 - RNA, 20 nt, Artificial Sequence
  Synthetic
ugcagaauuuguucacugag

SEQ ID NO: 215 - RNA, 20 nt, Artificial Sequence
  Synthetic
gugaggauugcagaauuugu

SEQ ID NO: 216 - RNA, 20 nt, Artificial Sequence
  Synthetic
ugaggauugcagaauuuguu

SEQ ID NO: 217 - RNA, 20 nt, Artificial Sequence
  Synthetic
gaggauugcagaauuuguuc

SEQ ID NO: 218 - RNA, 20 nt, Artificial Sequence
  Synthetic
aggauugcagaauuuguuca

SEQ ID NO: 219 - RNA, 20 nt, Artificial Sequence
  Synthetic
ggauugcagaauuuguucac

SEQ ID NO: 220 - RNA, 20 nt, Artificial Sequence
  Synthetic
gauugcagaauuuguucacu

SEQ ID NO: 221 - RNA, 20 nt, Artificial Sequence
  Synthetic
auugcagaauuuguucacug

SEQ ID NO: 222 - RNA, 20 nt, Artificial Sequence
  Synthetic
uugcagaauuuguucacuga

SEQ ID NO: 223 - RNA, 20 nt, Artificial Sequence
  Synthetic
ugcagaauuuguucacugag

SEQ ID NO: 224 - RNA, 20 nt, Artificial Sequence
  Synthetic
cccauaaaaguuuucucugc

SEQ ID NO: 225 - RNA, 20 nt, Artificial Sequence
  Synthetic
acccauaaaaguuuucucug

SEQ ID NO: 226 - RNA, 20 nt, Artificial Sequence
  Synthetic
aacccauaaaaguuuucucu

SEQ ID NO: 227 - RNA, 20 nt, Artificial Sequence
  Synthetic
ccauaaaaguuuucucugca

SEQ ID NO: 228 - RNA, 20 nt, Artificial Sequence
  Synthetic
cccauaaaaguuuucucugc

SEQ ID NO: 229 - RNA, 20 nt, Artificial Sequence
  Synthetic
acccauaaaaguuuucucug

SEQ ID NO: 230 - RNA, 20 nt, Artificial Sequence
  Synthetic
aacccauaaaaguuuucucu

SEQ ID NO: 231 - RNA, 20 nt, Artificial Sequence
  Synthetic
ccauaaaaguuuucucugca

SEQ ID NO: 232 - RNA, 20 nt, Artificial Sequence
  Synthetic
cccauaaaaguuuucucugc

SEQ ID NO: 233 - RNA, 20 nt, Artificial Sequence
  Synthetic
acccauaaaaguuuucucug

SEQ ID NO: 234 - RNA, 20 nt, Artificial Sequence
  Synthetic
aacccauaaaaguuuucucu

SEQ ID NO: 235 - RNA, 20 nt, Artificial Sequence
  Synthetic
ccauaaaaguuuucucugca

SEQ ID NO: 236 - RNA, 20 nt, Artificial Sequence
  Synthetic
cccauaaaaguuuucucugc

SEQ ID NO: 237 - RNA, 20 nt, Artificial Sequence
  Synthetic
acccauaaaaguuuucucug

SEQ ID NO: 238 - RNA, 20 nt, Artificial Sequence
  Synthetic
aacccauaaaaguuuucucu

SEQ ID NO: 239 - RNA, 20 nt, Artificial Sequence
  Synthetic
ggauugcagaauuuguucac

SEQ ID NO: 240 - RNA, 20 nt, Artificial Sequence
  Synthetic
ggauugcagaauuuguucac

SEQ ID NO: 241 - RNA, 20 nt, Artificial Sequence
  Synthetic
ggauugcagaauuuguucac

SEQ ID NO: 242 - RNA, 20 nt, Artificial Sequence
  Synthetic
ggauugcagaauuuguucac

SEQ ID NO: 243 - RNA, 20 nt, Artificial Sequence
  Synthetic
ggauugcagaauuuguucac

SEQ ID NO: 244 - RNA, 20 nt, Artificial Sequence
  Synthetic
ggauugcagaauuuguucac

SEQ ID NO: 245 - RNA, 20 nt, Artificial Sequence
  Synthetic
ggauugcagaauuuguucac

SEQ ID NO: 246 - RNA, 20 nt, Artificial Sequence
  Synthetic
ggauugcagaauuuguucac

SEQ ID NO: 247 - RNA, 20 nt, Artificial Sequence
  Synthetic
ggauugcagaauuuguucac

SEQ ID NO: 248 - RNA, 20 nt, Artificial Sequence
  Synthetic
ggauugcagaauuuguucac

SEQ ID NO: 249 - DNA, 20 bp, Artificial Sequence
  Synthetic
ggattgcagaatttgttcac

SEQ ID NO: 250 - RNA, 20 nt, Artificial Sequence
  Synthetic
ggauugcagaauuuguucac

SEQ ID NO: 251 - RNA, 20 nt, Artificial Sequence
  Synthetic
ggauugcagaauuuguucac

SEQ ID NO: 252 - RNA, 20 nt, Artificial Sequence
  Synthetic
ggauugcagaauuuguucac

SEQ ID NO: 253 - RNA, 20 nt, Artificial Sequence
  Synthetic
ggauugcagaauuuguucac

SEQ ID NO: 254 - RNA, 20 nt, Artificial Sequence
  Synthetic
ggauugcagaauuuguucac

SEQ ID NO: 255 - RNA, 20 nt, Artificial Sequence
  Synthetic
ggauugcagaauuuguucac

SEQ ID NO: 256 - RNA, 20 nt, Artificial Sequence
  Synthetic
ggauugcagaauuuguucac

SEQ ID NO: 257 - RNA, 20 nt, Artificial Sequence
  Synthetic
ggauugcagaauuuguucac

SEQ ID NO: 258 - RNA, 20 nt, Artificial Sequence
  Synthetic
ggauugcagaauuuguucac

SEQ ID NO: 259 - RNA, 20 nt, Artificial Sequence
  Synthetic
ggauugcagaauuuguucac

SEQ ID NO: 260 - RNA, 20 nt, Artificial Sequence
  Synthetic
ggauugcagaauuuguucac

SEQ ID NO: 261 - RNA, 20 nt, Artificial Sequence
  Synthetic
gauugcagaauuuguucacu

SEQ ID NO: 262 - RNA, 20 nt, Artificial Sequence
  Synthetic
gauugcagaauuuguucacu

SEQ ID NO: 263 - DNA, 20 bp, Artificial Sequence
  Synthetic
gattgcagaatttgttcact

SEQ ID NO: 264 - RNA, 20 nt, Artificial Sequence
  Synthetic
gauugcagaauuuguucacu

SEQ ID NO: 265 - RNA, 20 nt, Artificial Sequence
  Synthetic
gauugcagaauuuguucacu

SEQ ID NO: 266 - RNA, 20 nt, Artificial Sequence
  Synthetic
gauugcagaauuuguucacu

SEQ ID NO: 267 - RNA, 20 nt, Artificial Sequence
  Synthetic
gauugcagaauuuguucacu

SEQ ID NO: 268 - RNA, 20 nt, Artificial Sequence
  Synthetic
gauugcagaauuuguucacu

SEQ ID NO: 269 - RNA, 20 nt, Artificial Sequence
  Synthetic
gauugcagaauuuguucacu

SEQ ID NO: 270 - RNA, 20 nt, Artificial Sequence
  Synthetic
gauugcagaauuuguucacu

SEQ ID NO: 271 - RNA, 20 nt, Artificial Sequence
  Synthetic
gauugcagaauuuguucacu

SEQ ID NO: 272 - RNA, 20 nt, Artificial Sequence
  Synthetic
gauugcagaauuuguucacu

SEQ ID NO: 273 - RNA, 20 nt, Artificial Sequence
  Synthetic
gauugcagaauuuguucacu

SEQ ID NO: 274 - RNA, 20 nt, Artificial Sequence
  Synthetic
gauugcagaauuuguucacu

SEQ ID NO: 275 - RNA, 20 nt, Artificial Sequence
  Synthetic
ugcagaauuuguucacugag

SEQ ID NO: 276 - RNA, 20 nt, Artificial Sequence
  Synthetic
ugcagaauuuguucacugag

SEQ ID NO: 277 - DNA, 20 bp, Artificial Sequence
  Synthetic
tgcagaatttgttcactgag

SEQ ID NO: 278 - RNA, 20 nt, Artificial Sequence
  Synthetic
ugcagaauuuguucacugag

SEQ ID NO: 279 - RNA, 20 nt, Artificial Sequence
  Synthetic
ugcagaauuuguucacugag

SEQ ID NO: 280 - RNA, 20 nt, Artificial Sequence
  Synthetic
ugcagaauuuguucacugag

SEQ ID NO: 281 - RNA, 20 nt, Artificial Sequence
  Synthetic
ugcagaauuuguucacugag

SEQ ID NO: 282 - RNA, 20 nt, Artificial Sequence
  Synthetic
ugcagaauuuguucacugag

SEQ ID NO: 283 - RNA, 20 nt, Artificial Sequence
  Synthetic
ugcagaauuuguucacugag

SEQ ID NO: 284 - RNA, 20 nt, Artificial Sequence
  Synthetic
ugcagaauuuguucacugag

SEQ ID NO: 285 - RNA, 20 nt, Artificial Sequence
  Synthetic
ugcagaauuuguucacugag

SEQ ID NO: 286 - RNA, 20 nt, Artificial Sequence
  Synthetic
ugcagaauuuguucacugag

SEQ ID NO: 287 - RNA, 20 nt, Artificial Sequence
  Synthetic
ugcagaauuuguucacugag

SEQ ID NO: 288 - RNA, 20 nt, Artificial Sequence
  Synthetic
ugcagaauuuguucacugag

SEQ ID NO: 289 - RNA, 20 nt, Artificial Sequence
  Synthetic
ugcagaauuuguucacugag

SEQ ID NO: 290 - RNA, 19 nt, Artificial Sequence
  Synthetic
gauugcagaauuuguucac

SEQ ID NO: 291 - RNA, 18 nt, Artificial Sequence
  Synthetic
auugcagaauuuguucac

SEQ ID NO: 292 - RNA, 17 nt, Artificial Sequence
  Synthetic
uugcagaauuuguucac

SEQ ID NO: 293 - RNA, 19 nt, Artificial Sequence
  Synthetic
ggauugcagaauuuguuca

SEQ ID NO: 294 - RNA, 18 nt, Artificial Sequence
  Synthetic
ggauugcagaauuuguuc

SEQ ID NO: 295 - RNA, 17 nt, Artificial Sequence
  Synthetic
ggauugcagaauuuguu

SEQ ID NO: 296 - RNA, 20 nt, Artificial Sequence
  Synthetic
ccaaggaaucacacucacac

SEQ ID NO: 297 - RNA, 20 nt, Artificial Sequence
  Synthetic
caaggaaucacacucacaca

SEQ ID NO: 298 - RNA, 20 nt, Artificial Sequence
  Synthetic
aaggaaucacacucacacau

SEQ ID NO: 299 - RNA, 20 nt, Artificial Sequence
  Synthetic
aggaaucacacucacacauc

SEQ ID NO: 300 - RNA, 20 nt, Artificial Sequence
  Synthetic
gaaucacacucacacaucug

SEQ ID NO: 301 - RNA, 20 nt, Artificial Sequence
  Synthetic
aaucacacucacacaucugg

SEQ ID NO: 302 - RNA, 20 nt, Artificial Sequence
  Synthetic
aucacacucacacaucuggc

SEQ ID NO: 303 - RNA, 20 nt, Artificial Sequence
  Synthetic
ucacacucacacaucuggca

SEQ ID NO: 304 - RNA, 20 nt, Artificial Sequence
  Synthetic
ccaaggaaucacacucacac

SEQ ID NO: 305 - RNA, 20 nt, Artificial Sequence
  Synthetic
caaggaaucacacucacaca

SEQ ID NO: 306 - RNA, 20 nt, Artificial Sequence
  Synthetic
aaggaaucacacucacacau

SEQ ID NO: 307 - RNA, 20 nt, Artificial Sequence
  Synthetic
aggaaucacacucacacauc

SEQ ID NO: 308 - RNA, 20 nt, Artificial Sequence
  Synthetic
ggaaucacacucacacaucu

SEQ ID NO: 309 - RNA, 20 nt, Artificial Sequence
  Synthetic
gaaucacacucacacaucug

SEQ ID NO: 310 - RNA, 20 nt, Artificial Sequence
  Synthetic
aaucacacucacacaucugg

SEQ ID NO: 311 - RNA, 20 nt, Artificial Sequence
  Synthetic
aucacacucacacaucuggc

SEQ ID NO: 312 - RNA, 20 nt, Artificial Sequence
  Synthetic
ucacacucacacaucuggca

SEQ ID NO: 313 - RNA, 20 nt, Artificial Sequence
  Synthetic
ccaaggaaucacacucacac

SEQ ID NO: 314 - RNA, 20 nt, Artificial Sequence
  Synthetic
caaggaaucacacucacaca

SEQ ID NO: 315 - RNA, 20 nt, Artificial Sequence
  Synthetic
aaggaaucacacucacacau

SEQ ID NO: 316 - RNA, 20 nt, Artificial Sequence
  Synthetic
aggaaucacacucacacauc

SEQ ID NO: 317 - RNA, 20 nt, Artificial Sequence
  Synthetic
ggaaucacacucacacaucu

SEQ ID NO: 318 - RNA, 20 nt, Artificial Sequence
  Synthetic
gaaucacacucacacaucug

SEQ ID NO: 319 - RNA, 20 nt, Artificial Sequence
  Synthetic
aaucacacucacacaucugg

SEQ ID NO: 320 - RNA, 20 nt, Artificial Sequence
  Synthetic
aucacacucacacaucuggc

SEQ ID NO: 321 - RNA, 20 nt, Artificial Sequence
  Synthetic
ucacacucacacaucuggca

SEQ ID NO: 322 - RNA, 20 nt, Artificial Sequence
  Synthetic
ccaaggaaucacacucacac

SEQ ID NO: 323 - RNA, 20 nt, Artificial Sequence
  Synthetic
caaggaaucacacucacaca

SEQ ID NO: 324 - RNA, 20 nt, Artificial Sequence
  Synthetic
aaggaaucacacucacacau

SEQ ID NO: 325 - RNA, 20 nt, Artificial Sequence
  Synthetic
aggaaucacacucacacauc

SEQ ID NO: 326 - RNA, 20 nt, Artificial Sequence
  Synthetic
ggaaucacacucacacaucu

SEQ ID NO: 327 - RNA, 20 nt, Artificial Sequence
  Synthetic
gaaucacacucacacaucug

SEQ ID NO: 328 - RNA, 20 nt, Artificial Sequence
  Synthetic
aaucacacucacacaucugg

SEQ ID NO: 329 - RNA, 20 nt, Artificial Sequence
  Synthetic
aucacacucacacaucuggc

SEQ ID NO: 330 - RNA, 20 nt, Artificial Sequence
  Synthetic
ucacacucacacaucuggca

SEQ ID NO: 331 - RNA, 20 nt, Artificial Sequence
  Synthetic
aucacacucacacaucuggc

SEQ ID NO: 332 - RNA, 20 nt, Artificial Sequence
  Synthetic
ucacacucacacaucuggca

SEQ ID NO: 333 - RNA, 20 nt, Artificial Sequence
  Synthetic
ugcagaauuuguucacugag

SEQ ID NO: 334 - RNA, 20 nt, Artificial Sequence
  Synthetic
ugcagaauuuguucacugag

SEQ ID NO: 335 - RNA, 20 nt, Artificial Sequence
  Synthetic
ugcagaauuuguucacugag

SEQ ID NO: 336 - RNA, 19 nt, Artificial Sequence
  Synthetic
gcagaauuuguucacugag

SEQ ID NO: 337 - RNA, 18 nt, Artificial Sequence
  Synthetic
cagaauuuguucacugag

SEQ ID NO: 338 - RNA, 19 nt, Artificial Sequence
  Synthetic
ugcagaauuuguucacuga

SEQ ID NO: 339 - RNA, 18 nt, Artificial Sequence
  Synthetic
ugcagaauuuguucacug

SEQ ID NO: 340 - RNA, 18 nt, Artificial Sequence
  Synthetic
gcagaauuuguucacuga

SEQ ID NO: 341 - DNA, 15 bp, Artificial Sequence
  Synthetic
gttattgggcttagg

SEQ ID NO: 342 - DNA, 15 bp, Artificial Sequence
  Synthetic
gttattggtttttat